(19) United States
(12) Patent Application Publication
(10) Pub. No.: US 2025/0215081 A1
(43) Pub. Date: Jul. 3, 2025

(54) ANTI-CD19 AGENT AND B CELL TARGETING AGENT COMBINATION THERAPY FOR TREATING B CELL MALIGNANCIES

(71) Applicant: NOVARTIS AG, Basel (CH)

(72) Inventors: Kimberly Marie AARDALEN, Norwell, MA (US); Regis CEBE, Saint-Louis (FR); Dattananda CHELUR, Chesterbrook, PA (US); Glenn DRANOFF, Concord, MA (US); Brian Walter GRANDA, Salisbury, MA (US); Nadia HASSOUNAH, Boston, MA (US); Connie HONG, Somerville, MA (US); Sunyoung JANG, Cambridge, MA (US); Haihui LU, Winchester, MA (US); Amy RAYO, Cambridge, MA (US); Darko SKEGRO, Riehen (CH); Janghee WOO, Short Hills, NJ (US)

(21) Appl. No.: 18/035,472

(22) PCT Filed: Nov. 4, 2021
(86) PCT No.: PCT/IB2021/060216

§ 371 Date: May 4, 2023

Related U.S. Application Data
(60) Provisional application No. 63/110,490, filed on Nov. 6, 2020.
(60) Provisional application No. 63/110,501, filed on Nov. 6, 2020.
(60) Provisional application No. 63/114,370, filed on Nov. 16, 2020.
(60) Provisional application No. 63/114,371, filed on Nov. 16, 2020.
(60) Provisional application No. 63/147,488, filed on Feb. 9, 2021.
(60) Provisional application No. 63/147,501, filed on Feb. 9, 2021.

(51) Int. Cl. C07K 16/28; A61K 39/00; A61P 35/00

(52) U.S. Cl. CPC C07K 16/2809; A61P 35/00; C07K 16/2803; C07K 16/2824; A61K 2039/505; C07K 2317/24; C07K 2317/31; C07K 2317/52; C07K 2317/569; C07K 2317/92

(57) ABSTRACT

The present disclosure provides combinations of anti-CD19 agents and B cell targeting agents and methods of treating subjects having B cell malignancies with combinations of anti-CD19 agents and a B cell targeting agents.

### 1. CROSS-REFERENCE TO RELATED APPLICATIONS

This application claims the priority benefit of U.S. provisional application nos. 63/110,501, filed Nov. 6, 2020, 63/114,370, filed Nov. 16, 2020, 63/114,371, filed Nov. 16, 2020, 63/147,488, filed Feb. 9, 2021, 63/147,501, filed Feb. 9, 2021, and 63/110,490, filed Nov. 6, 2020, the contents of each of which are incorporated herein in their entireties by reference thereto.

### 2. SEQUENCE LISTING

The instant application contains a Sequence Listing which has been submitted electronically in ASCII format and is hereby incorporated by reference in its entirety. Said ASCII copy, created on Nov. 4, 2021, is named NOV-013WO_SL.txt and is 704,029 bytes in size.

### 3. FIELD OF INVENTION

The disclosure generally relates to combinations of anti-CD19 agents and B cell targeting agents, and their use for treating B cell malignancies.

### 4. BACKGROUND

B cells express a wide array of cell surface molecules during their differentiation and proliferation. CD19 is a pan-B cell membrane glycoprotein that is expressed from early stages of pre-B cell development through terminal differentiation, regulating B lymphocyte development and function. Expression of CD19 was identified on the vast majority of Non-Hodgkin lymphoma (NHL) and on leukemias, including Chronic Lymphocytic Leukemia (CLL), Acute Lymphoblastic Leukemia (ALL) and Waldenstrom's Macroglobulinemia (WM).

A few anti-CD19 agents are approved for treating B cell malignancies, for example, blinatumomab (marketed by Amgen as BLINCYTO®), which is a CD19-CD3 bispecific T cell engager that is approved for the treatment of the treatment of ALL, tisagenlecleucel (marketed by Novartis as KYMRIAH®), which is a chimeric antigen receptor (CAR) T cell composition that is approved for the treatment of ALL, axicabtagene ciloleucel (marketed by Gilead as Gilead as YESCARTA®), which is a CAR T cell composition approved for diffuse large B-cell lymphoma (DLBCL), and brexucabtagene autoleucel (marketed by Gilead as TECARTUS®), which is a CAR T cell composition approved for mantle cell lymphoma (MCL). However, some patients treated with blinatumomab and CD19-specific CAR T therapy develop cytokine release syndrome (CRS). Teachey et al., 2013, Blood, 121(26): 5154-5157; Park et al., 2018, Clin Infect Dis. 2018 Aug. 15; 67(4): 533-540. CRS is a systemic inflammatory response that can produce symptoms ranging from mild, flu-like symptoms, to severe life-threatening inflammatory responses. Shimabukuro-Vornhagen et al., 2018, J Immunother Cancer. 6:56.

Despite major improvements in cancer therapy, B cell malignancies such as the B cell subtypes of non-Hodgkin's lymphomas, and chronic lymphocytic leukemia, are major contributors of cancer-related deaths. Accordingly, there is still a need for further therapeutic agents and methods for the treatment of B cell malignancies and management of CRS associated with anti-CD19 agents.

### 5. SUMMARY

The disclosure provides combinations of anti-CD19 agents and B cell targeting agents and methods of using such combinations for treating B cell malignancies. Without being bound by theory, it is believed that CRS associated with anti-CD19 agents can be mitigated by depleting normal B cells with a B cell targeting agent. Again without being bound by theory, it is believed that the therapeutic efficacy of an anti-CD19 agent can be enhanced when administered in combination with a B cell targeting agent.

Accordingly, in one aspect, the disclosure provides a method of treating a subject having a B cell malignancy, by administering an anti-CD19 agent and a B cell targeting agent to the subject. In some embodiments, the B cell targeting agent is administered prior to administration of the anti-CD19 agent. Without being bound by theory, it is believed that cytokine release by normal B cells is an important driver in CRS, and it is believed that depleting normal B cells in a subject with a B cell targeting agent prior to administering an anti-CD19 agent to the subject can reduce the severity of CRS experienced by the subject.

In another aspect, the disclosure provides combinations of anti-CD19 agents and B cell targeting agents. Such combinations can be used, for example, in methods of treating a subject having a B cell malignancy (e.g., a NHL such as DLBCL or MCL). In some embodiments, the subject has a NHL, for example DLBCL or MCL, and (i) has failed at least one prior line (and optionally up to five prior lines) of standard of care therapy, e.g., an anti-CD20 therapy such as rituximab and/or (ii) is intolerant to or ineligible for one or more other approved therapies, e.g., autologous stem cell transplant (ASCT) and/or (iii) is a non-responder to a chimeric antigen receptor (CAR) T cell therapy. The NHL can be relapsed and/or refractory.

In further aspects, the disclosure provides anti-CD19 agents for use in combination with B cell targeting agents and B cell targeting agents for use in combination with anti-CD19 agents, for example, for use in treating a subject having a B cell malignancy (e.g., a NHL such as DLBCL or MCL). In some embodiments, the subject has a NHL, for example DLBCL or MCL, and (i) has failed at least one prior line (and optionally up to five prior lines) of standard of care therapy, e.g., an anti-CD20 therapy such as rituximab and/or (ii) is intolerant to or ineligible for one or more other approved therapies, e.g., autologous stem cell transplant (ASCT) and/or (iii) is a non-responder to a chimeric antigen receptor (CAR) T cell therapy. The NHL can be relapsed and/or refractory.

The anti-CD19 agents used in the methods and combinations of the disclosure can be CD19 binding molecules that specifically bind to human CD19, e.g., antibodies, antigen-binding fragments thereof, and multispecific molecules that specifically bind to human CD19. Alternatively, the anti-CD19 agent can be a population of cells that expresses a chimeric antigen receptor (“CAR”) molecule that binds CD19.

In some aspects, the CD19 binding molecules are monospecific CD19 binding molecules (e.g., antibodies and antigen-binding fragments thereof) comprising a CD19 antigen-binding domain or antigen-binding module (“ABM”). Exemplary CD19 binding molecules, which can be monospecific, are described in Section 7.2 and specific embodiments 2 to 39, infra.

In other aspects, the CD19 binding molecules are multispecific binding molecules (“MBMs”) comprising a CD19 ABM. In certain embodiments, the MBMs are bispecific binding molecules (“BBMs”). The BBMs comprise a first ABM that specifically binds to human CD19 (“ABM1” or “CD19 ABM”) and a second ABM that specifically binds to a second antigen (“ABM2”), e.g., human CD3 or other component of a T cell receptor (TCR) complex (sometimes referred to herein as a “TCR ABM”). The terms ABM1, ABM2, CD19 ABM, and TCR ABM are used merely for convenience and are not intended to convey any particular configuration of a BBM. In some embodiments, a TCR ABM binds to CD3 (referred to herein a “CD3 ABM” or the like). Accordingly, disclosures relating to ABM2 and TCR ABMs are also applicable to CD3 ABMs. Such multispecific molecules can be used to direct CD3+ effector T cells to CD19+ sites, thereby allowing the CD3+ effector T cells to attack and lyse CD19+ cells and tumors.

In other embodiments, the MBMs are trispecific binding molecules (“TBMs”) that engage CD19, CD3 or other component of a TCR complex on T-cells, and either CD2 or a human tumor-associated antigen (“TAA”), for example a B cell antigen other than CD19. The TBMs comprise at least three antigen-binding modules (“ABMs”) that can bind (i) CD19 (ABM1), (ii) a component of a TCR complex (ABM2), and (iii) either CD2 or a TAA (ABM3). TBMs that bind to (1) human CD19, (2) CD3 or other component of a TCR complex, and (3) CD2 are referred to herein as “Type 1 TBMs” for convenience. TBMs that bind to (1) human CD19, (2) CD3 or other component of a TCR complex, and (3) a TAA are referred to herein as “Type 2 TBMs” for convenience.

Without being bound by theory, the inventors believe that combining CD2− and TCR complex-engagement in a Type 1 TBM can stimulate both a primary signaling pathway that promotes T-cell mediated lysis of tumor cells (by clustering TCRs, for example) and a second co-stimulatory pathway to induce T-cell proliferation and potentially overcome anergy. Also without being bound by theory, it is believed that engaging a TAA in addition to CD19 and a component of a TCR complex a Type 2 TBM will improve the clinical outcomes of RTCC therapy of B cell malignancies by targeting a greater number of cancerous B cells than using bispecific engagers that target only a CD19 and a TCR complex component.

Accordingly, in some embodiments, the CD19 binding molecules used in the methods and combinations of the disclosure are Type 1 TBMs that bind to (1) human CD19, (2) CD3 or other component of a TCR complex, and (3) CD2.

In other embodiments, the CD19 binding molecules used in the methods and combinations of the disclosure are Type 2 TBMs that bind to (1) human CD19, (2) CD3 or other component of a TCR complex, and (3) a TAA.

Unless expressly indicated otherwise or unless the context dictates otherwise, a reference to TBMs in the present disclosure applies to both Type 1 and Type 2 TBMs.

Features of exemplary MBMs are described in Section 7.2 and specific embodiments 40 to 605, infra.

Further exemplary CD19 binding molecules that can be used in the methods and combinations of the disclosure are described in Section 7.2 and specific embodiments 614 to 626, infra.

In some aspects, the anti-CD19 agents used in the methods and combinations of the disclosure are populations of cells that express CAR molecules that bind CD19. Features of exemplary CARs and populations of cells that express CAR molecules are described in Section 7.3 and specific embodiments 627 to 700, infra.

In some aspects, the B cell targeting agent is a B-cell activating factor receptor (BAFFR) binding molecule, a CD20 binding molecule, a CD22 binding molecule, or a B-cell activating factor (BAFF) binding molecule. Exemplary features of B cell targeting agents are described in Section 7.4 and specific embodiments 701 to 741, infra.

Exemplary B cell malignancies and patient populations suitable for treatment using the methods and compositions described herein are described in Section 7.6 and specific embodiments 760 to 807, infra.

The anti-CD19 agents described throughout can be administered to a subject as a combination treatment. For example, the combination can comprise an anti-CD19 agent and a B cell targeting agent. The anti-CD19 agent, in some embodiments can be a CD19 binding molecule.

In some embodiments (e.g., as used in a combination), the CD19 binding molecule can comprise a CDR-H1, a CDR-H2, and a CDR-H3 having the amino acid sequences of SEQ ID NO:1, SEQ ID NO:2, and SEQ ID NO:3, and a CDR-L1, a CDR-L2, and a CDR-L3 having the amino acid sequences of SEQ ID NO:14, SEQ ID NO:15, and SEQ ID NO:16. In other embodiments, the CD19 binding molecule can comprise a CDR-H1, a CDR-H2, and a CDR-H3 having the amino acid sequences of SEQ ID NO:4, SEQ ID NO:5, and SEQ ID NO:6, and a CDR-L1, a CDR-L2, and a CDR-L3 having the amino acid sequences of SEQ ID NO:17, SEQ ID NO:18, and SEQ ID NO:19. The CD19 binding molecule can also comprise a CDR-H1, a CDR-H2, and a CDR-H3 having the amino acid sequences of SEQ ID NO:7, SEQ ID NO:8, and SEQ ID NO:9, and a CDR-L1, a CDR-L2, and a CDR-L3 having the amino acid sequences of SEQ ID NO:20, SEQ ID NO:21, and SEQ ID NO:22. In certain embodiments, the CD19 binding can comprise a CDR-H1, a CDR-H2, and a CDR-H3 having the amino acid sequences of SEQ ID NO:10, SEQ ID NO:11, and SEQ ID NO:12, and a CDR-L1, a CDR-L2, and a CDR-L3 having the amino acid sequences of SEQ ID NO:23, SEQ ID NO:24, and SEQ ID NO:25.

The CD19 binding molecule can comprise a VH having the amino acid sequence of SEQ ID NO:13. The CD19 binding molecule can also comprise a VL having the amino acid sequence of SEQ ID NO:26. The CD19 binding molecule can also comprise both a VH having the amino acid sequence of SEQ ID NO:13 and a VL having the amino acid sequence of SEQ ID NO:26.

The CD19 binding molecule can also be a multispecific binding molecule (MBM). For example, the CD19 binding molecule can comprise (a) an antigen-binding module 1 (ABM1) that binds specifically to CD19; and (b) an antigen-binding module 2 (ABM2) that binds specifically to a different target molecule (e.g., a component of a human T-cell receptor (TCR) complex (such as CD3)). The CD19 binding molecule can be a trispecific binding molecule (TBM) that comprises an antigen-binding module 3 (ABM3) that binds specifically to a target molecule other than CD19. For example, if the CD19 binding molecule is a TBM, then in some examples, the ABM2 can bind specifically to a component of a human T-cell receptor (TCR) complex and ABM3 can bind specifically to human CD2.

The CD19 binding molecule in some embodiments can be trivalent. The CD19 binding molecule can be configured in one of multiple ways, for example, as any one of the configurations depicted in FIGS. 2A-2P. For example, the CD19 binding molecule can have the configuration as depicted in FIG. 21. The CD19 binding molecule can also have the configuration referred to as T2 in Section 7.2.4.1.

The CD19 binding molecule can have an ABM3 that binds specifically to human CD2. In some embodiments, the ABM3 is a non-immunoglobulin scaffold based ABM. In some embodiments, the ABM3 can comprise a receptor binding domain of a CD2 ligand. In some embodiments, the ABM3 is a CD58 moiety. The CD58 moiety used can comprise the amino acid sequence of CD58-6 as set forth in Table 12.

The CD19 binding molecule can also comprise unique Fc domains. For examples, the CD19 binding molecule can comprise a first variant Fc region and a second variant Fc region forming an Fc domain. The first variant Fc region and the second variant Fc region can together form an Fc heterodimer. In some embodiments, the first and second variant Fc regions can comprise the amino acid substitutions amino acid substitutions T366W: T366S/L368A/Y407V. In some embodiments, the Fc domain is an Fc heterodimer that comprises knob-in-hole (“KIH”) modifications. In some embodiments, the Fc domain has altered effector function. In some embodiments, the Fc domain can have altered binding to one or more Fc receptors. Some mutations of the Fc domain can be a silencing mutation. For example, one or more of the mutations can lead to a silent IgG1. In some embodiments, the mutation can comprising a D265A mutation. In other embodiments, the mutations can comprise D265A and P329A mutations. In some embodiments, the Fc domain of the CD19 binding molecule is a human IgG1 Fc domain which comprises: (a) a first CH3 domain comprising the modification T366W; and (b) a second CH3 domain that heterodimerizes with the first CH3 domain and comprises the modifications T366S, L368A and Y407V. In some embodiments, the the Fc domain of the CD19 binding molecule comprises a human IgG1 Fc domain modified by substituting the aspartate residue at position 265 with an alanine residue, the asparagine residue at position 297 with an alanine residue and the proline residue at position 329 with an alanine residue (D265A/N297A/P329A).

In some specific embodiments, the CD19 binding molecule is a trispecific binding molecule (TBM) that comprises (a) an antigen-binding module 1 (ABM1) that binds specifically to CD19 and comprises CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19; (b) an antigen-binding module 2 (ABM2) that binds specifically to a component of a human T-cell receptor (TCR) complex; and (c) an antigen-binding module 3 (ABM3) that binds specifically to human CD2. This CD19 binding molecule can be trivalent. The ABM1 can be a Fab. The CD19 binding molecule can also have an ABM1 that comprises a VH having the amino acid sequence of SEQ ID NO:13 and a VL having the amino acid sequence of SEQ ID NO:26.

The CD19 binding molecule can have a component of the TCR complex that binds to CD3. The CD19 binding molecule's ABM2 can be an anti-CD3 antibody or an antigen-binding domain thereof. For example, the ABM2 can comprise the CDR sequences of CD3hi. The heavy chain sequences of CD3hi can be as set forth in Table 9A. In some embodiments, the anti-CD3 antibody or antigen-binding domain thereof is in the form of an scFv. For example, the ABM2 can comprise the amino acid sequence of the scFv designated as CD3hi in Table 9A. The CD19 binding molecule can comprise an ABM3 that is a CD58 moiety. As an example, the CD58 moiety can be an amino acid sequence of CD58-6 as set forth in Table 12. The CD19 binding molecule used in the combinations and/or disclosed throughout can also comprise an Fc domain. In the Fc domain, the first variant Fc region and a second variant Fc region can together form an Fc heterodimer.

In a specific embodiment, the CD19 binding molecule is a trispecific binding molecule (TBM) comprising (a) an antigen-binding module 1 (ABM1) that binds specifically to CD19 and which is a Fab comprising CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19; (b) an antigen-binding module 2 (ABM2) that binds specifically to CD3 and which comprises the amino acid sequence of the scFv designated as CD3hi in Table 9A; (c) an antigen-binding module 3 (ABM3) that binds specifically to human CD2 and which comprises the amino acid sequence of CD58-6 as set forth in Table 12; and (d) an Fc domain.

In a specific embodiment, the CD19 binding molecule is a trispecific binding molecule (TBM) comprising (a) an antigen-binding module 1 (ABM1) that binds specifically to CD19 and which is a Fab comprising a CDR-H1, a CDR-H2, and a CDR-H3 having the amino acid sequences of SEQ ID NO:30, SEQ ID NO:31, and SEQ ID NO:32, and a CDR-L1, a CDR-L2, and a CDR-L3 having the amino acid sequences of SEQ ID NO:43, SEQ ID NO:44, and SEQ ID NO:45; (b) an antigen-binding module 2 (ABM2) that binds specifically to CD3 and which comprises the amino acid sequence of the scFv designated as CD3hi in Table 9A; (c) an antigen-binding module 3 (ABM3) that binds specifically to human CD2 and which comprises the amino acid sequence of CD58-6 as set forth in Table 12; and (d) an Fc domain.

In another specific embodiment, the CD19 binding molecule is a trispecific binding molecule (TBM) comprising (a) an antigen-binding module 1 (ABM1) that binds specifically to CD19 and which is a Fab comprising: (i) CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19; (b) an antigen-binding module 2 (ABM2) that binds specifically to CD3 and which comprises the amino acid sequence of the scFv designated as CD3hi in Table 9A; (c) an antigen-binding module 3 (ABM3) that binds specifically to human CD2 and which comprises the amino acid sequence of CD58-6 as set forth in Table 12; and (d) an Fc domain. The ABM1 of the CD19 binding molecule can comprise a CDR-H1, a CDR-H2, and a CDR-H3 having the amino acid sequences of SEQ ID NO:4, SEQ ID NO:5, and SEQ ID NO:6, and a CDR-L1, a CDR-L2, and a CDR-L3 having the amino acid sequences of SEQ ID NO:17, SEQ ID NO:18, and SEQ ID NO:19. In some embodiments, ABM1 can comprise a VH having the amino acid sequence of SEQ ID NO:13 and a VL having the amino acid sequence of SEQ ID NO:26.

In another specific embodiment, the CD19 binding molecule comprises a first half antibody comprising (a) an antigen-binding module 1 (ABM1) that binds specifically to CD19; (b) an antigen-binding module 2 (ABM2) that binds specifically to CD3 and which comprises an scFv; (c) an Fc region; and a second half antibody comprising an antigen-binding module 3 (ABM3) that binds specifically to human CD2 and which comprises a CD58 IgV domain and (d) an Fc region, where the Fc region in the first half antibody and the Fc region in the second half antibody form a Fc heterodimer. The CD19 binding molecule can comprise a first half antibody which comprises a heavy chain comprising the amino acid sequence of SEQ ID NO:63; and a light chain comprising the amino acid sequence of SEQ ID NO:64; and a second half antibody comprising the amino acid sequence of SEQ ID NO:65. The CD19 binding molecule used in the combination and/or as disclosed throughout can comprise a first half antibody which comprises a heavy chain comprising the amino acid sequence of SEQ ID NO:74; and a light chain comprising the amino acid sequence of SEQ ID NO:64; and a second half antibody comprising the amino acid sequence of SEQ ID NO:75 or SEQ ID NO:86. In some embodiments, the CD19 binding molecule used in the combination and/or as disclosed throughout can comprise a first half antibody which comprises a heavy chain comprising the amino acid sequence of SEQ ID NO:74; and a light chain comprising the amino acid sequence of SEQ ID NO:64; and a second half antibody comprising the amino acid sequence of SEQ ID NO:86.

In one embodiment, the CD19 binding molecule comprises (a) first half antibody heavy chain whose amino acid sequence comprises the amino acid sequence of SEQ ID NO:63 and a Fc sequence; (b) a first half antibody light chain whose amino acid sequence comprises the amino acid sequence of SEQ ID NO:64; (c) a second half antibody whose amino acid sequence comprises the amino acid sequence of SEQ ID NO:65 and a Fc sequence.

In another embodiment, the CD19 binding molecule can comprise (a) a first polypeptide whose amino acid sequence comprises the amino acid sequence of SEQ ID NO:74; (b) a second polypeptide whose amino acid sequence comprises the amino acid sequence of SEQ ID NO:64; and (c) a third polypeptide whose amino acid sequence comprises the amino acid sequence of SEQ ID NO:75 or SEQ ID NO:86.

In some embodiments, the CD19 binding molecule can comprise (a) a first polypeptide whose amino acid sequence comprises the amino acid sequence of SEQ ID NO:74; (b) a second polypeptide whose amino acid sequence comprises the amino acid sequence of SEQ ID NO:64; and (c) a third polypeptide whose amino acid sequence comprises the amino acid sequence of SEQ ID NO:86.

As described throughout, the combination can comprise an anti-CD19 agent and a B cell targeting agent. In some embodiments, the B cell targeting agent is a B cell depleting agent. In some embodiments, the B cell targeting agent is a BAFF receptor (BAFFR) binding molecule. For example, the BAFFR binding molecule is an antibody or antigen-binding domain thereof. In this embodiment, the BAFFR binding molecule can comprise a CDR-H1, a CDR-H2, a CDR-H3 having the amino acid sequences of ianalumab set forth in Table 18, and a CDR-L1, a CDR-L2, and a CDR-L3 having the amino acid sequences of ianalumab set forth in Table 18. The BAFFR binding molecule can comprise a heavy chain variable region (VH) and a light chain variable region (VL) having the VH and VL sequences of ianalumab set forth in Table 18. In a specific example, the BAFFR binding molecule is ianalumab.

The anti-CD19 agent and the B cell targeting agent can be separate molecules. In some embodiments, the anti-CD19 agent and the B cell targeting agent can be formulated in separate pharmaceutical compositions.

Provided herein is also a combination comprising an anti-CD19 agent as described herein and a B cell targeting agent as described herein for use in treating a subject having a B cell malignancy. The combination described throughout can be used in a method for treating a subject having a B cell malignancy. The method can comprise administering the anti-CD19 agent and a B cell targeting agent as described throughout. Regarding timing of administration, the B cell targeting agent can be administered to the subject one or more times prior to administering the anti-CD19 agent to the subject for the first time. The method of administration can also comprise simultaneously administering the anti-CD19 agent and the B cell targeting agent. In some embodiments, the B cell malignancy is diffuse large B-cell lymphoma (DLBCL). For example, the B cell malignancy can be relapsed and/or refractory diffuse large B-cell lymphoma (DLBCL). In some embodiments, the B cell malignancy can be acute lymphoblastic leukemia (ALL). For example, the B cell malignancy can be relapsed and/or refractory ALL. The combination of anti-CD19 agent and the B cell targeting agent can comprise further therapeutic agents as described herein.

### 6. BRIEF DESCRIPTION OF THE FIGURES

FIGS. 1A-1AH: Exemplary BBM configurations. FIG. 1A illustrates components of the exemplary BBM configurations illustrated in FIGS. 1B-1AH. Not all regions connecting the different domains of each chain are illustrated (e.g., the linker connecting the VH and VL domains of an scFv, the hinge connecting the CH2 and CH3 domains of an Fc domain, etc., are omitted). FIGS. 1B-1F illustrate bivalent BBMs; FIGS. 1G-1Z illustrate trivalent BBMs; FIGS. 1AA-1AH illustrate tetravalent BBMs.

FIGS. 2A-2V: Exemplary TBM configurations. FIG. 2A illustrates components of the exemplary TBM configurations illustrated in FIGS. 2B-2V. Not all regions connecting the different domains of each chain are illustrated (e.g., the linker connecting the VH and VL domains of an scFv, the hinge connecting the CH2 and CH3 domains of an Fc, etc., are omitted). FIG. 2B-2P illustrates trivalent TBMs; FIGS. 2Q-2S illustrate tetravalent TBMs; FIG. 2T illustrates a pentavalent TBM, and FIGS. 2U-2V illustrate hexavalent TBMs.

FIGS. 3A-3C: Schematics of the bispecific (FIG. 3A and FIG. 3C) and trispecific (FIG. 3B) constructs of Example 1.

FIGS. 4A-4B: Ability of CD19 BBMs to elicit redirected T-cell cytotoxic activity (RTCC) against CD19+ target cells. Both NEG258-based and NEG218-based BBMs mediated RTCC activity against CD19+ target cell lines. Nalm6-luc (FIG. 4A) and Karpas422-luc (FIG. 4B) cells were co-cultured with expanded T cells in the presence of serial diluted BBMs at an effector cell: target cell (E:T) ratio of 3:1. Luminescence signal was measured after 24h of incubation.

FIGS. 5A-5B: Ability of CD19 BBMs to elicit T-cell proliferation. Both NEG258-based and NEG218-based BBMs induced T cell proliferation. Karpas422-luc (FIG. 5A) and Nalm6-luc (FIG. 5B) cells were co-cultured with expanded T cells in the presence of serial diluted BBMs at an E:T ratio of 1:1. Luminescence signal was measured after 96h of incubation.

FIGS. 6A-6F: Ability of CD19 TBMs to elicit CD2 dependent T cell activation. CD2 knock out attenuated advantage of trispecific constructs. FIGS. 6A-6B show representative flow cytometry analysis of CD2 expression on JNL CD2 WT (FIG. 6A) and KO (FIG. 6B) cells. Staining by the anti-CD2 mAb (dot filled histogram) is overlaid with that of the mIgG1 isotype control (diagonal line filled histogram) or unstained (open histogram). FIGS. 6C-6F show data for JNL CD2+(FIG. 6C-6D) and CD2− (FIG. 6E-6F) cells co-cultured with CD19+ target cells in the presence of serial diluted BBMs and TBMs at an E:T ratio of 3:1. Luminescence signal was measured after 24h of incubation.

FIGS. 7A-7B: Binding of CD19 TBMs to cyno B cells. FIG. 7A shows data for a TBM with a NEG218-based CD19 binding arm and FIG. 7B shows data for a TBM with a NEG258-based CD19 binding arm.

FIGS. 8A-8H: Ability of CD19 TBMs to induce T cell activation upon cyno B cell depletion in PBMCs. In FIG. 8A, PBMCs were isolated from cyno monkey whole blood using ficoll gradient centrifugation and were incubated with bi or trispecific constructs for overnight. Samples were harvested and simultaneously stained for CD3 and CD20 to identify B and T cells within the PBMC population. Percentage of B cell depletion was calculated as described in Section 8.6.1. FIGS. 8B-8H show the results of FACS analysis of CD69 and CD25 expression on CD3⁺ T cells to determine single (CD69⁺CD25⁻ or CD69⁻CD25⁺) or double-positive cells (CD69⁺CD25⁺). FIG. 8B: untreated (media only); FIGS. 8C-8E: CD3hi TSP1L; FIGS. 8F-8H: CD3hi TSP1.

FIGS. 9A-9P: Ability of NEG258- and NEG218-based TBMs to induce redirected T cell cytotoxicity by human donor cells against Nalm6 (FIGS. 9A-9H) and Karpas422 (FIGS. 9I-9P) target cells.

FIGS. 10A-10P: Ability of NEG258- and NEG218-based TBMs with different CD3 affinities to induce redirected T cell cytotoxicity by human donor cells against Nalm6 (FIGS. 10A-10H) and Karpas422 (FIGS. 10I-10P) target cells.

FIGS. 11A-11L: Ability of NEG258-based TBMs that include a CD2-binding arm and those that include a control lysozyme binding arm to induce redirected T cell cytotoxicity by human donor cells against Nalm6 (FIGS. 11A-11H) and Karpas422 (FIGS. 11I-11L) target cells.

FIGS. 12A-12C: Induction of T cell cytokine release by NEG258- and NEG218-based TBMs. FIG. 12A: IFN-γ; FIG. 12B: TNF-α; FIG. 12C: IL2.

FIGS. 13A-13C: Binding of NEG258- and NEG218-based TBMs to murine 300.19 cell lines that overexpress human CD19 (FIG. 13A) or cyno CD19 (FIG. 13B). The TBMs show negligible binding to the wild type 300.19 cell line (FIG. 13C).

FIG. 14: A schematic representation of CD58.

FIG. 15: Redirected T cell cytotoxicity by TBMs containing CD58 variant sequences.

FIG. 16: Antigen-independent T-cell activation by TBMs containing CD58 variant sequences. Data expressed as relative luminescence units (RLU).

FIGS. 17A-17H: CD19 and CD58 expression on various cell lines: FIGS. 17A-17B: CD19 and CD58 expression, respectively, on OCI-LY-19 cells; FIGS. 17C-17D: CD19 and CD58 expression, respectively, on Karpas-422 cells; FIGS. 17E-17F: CD19 and CD58 expression, respectively, on Toledo cells; FIGS. 17G-17H: CD19 and CD58 expression, respectively, on Nalm-6 cells.

FIGS. 18A-18B: Ability of NEG258-based TBMs and BBM to induce redirected T cell cytotoxicity by human donor cells against Karpas422 target cells. FIG. 18A and FIG. 18B show data using T cells from two different donors.

FIGS. 19A-19F: Induction of T cell cytokine release by NEG258-based TBMs and BBM. FIGS. 19A-19B: IFN-γ (donor 1 and donor 2, respectively); FIGS. 19C-19D: IL-2 (donor 1 and donor 2, respectively); FIGS. 19E-19F: TNF-α (donor 1 and donor 2, respectively). Triangles on X-axis indicate decreasing concentration of constructs from left to right in the figures.

FIG. 20: NEG258-based TBM and BBM binding to T cells.

FIGS. 21A-21C: NEG258-based TBM and BBM mediated T cell proliferation. FIG. 21A: T cell proliferation in OC-LY-19 co-culture; FIG. 21B: T cell proliferation in Karpas422 co-culture; FIG. 21C: T cell proliferation in Toledo co-culture.

FIGS. 22A-22B: Ability of NEG258-based TBMs and BBM to induce redirected T cell cytotoxicity by human donor cells against Karpas422 target cells. FIG. 22A and FIG. 22B show data using T cells from two different donors.

FIGS. 23A-23J: Ability of NEG258-based TBMs and BBM to induce redirected T cell cytotoxicity by human donor cells against various target cells. FIGS. 23A-23B: OC-LY-19 (donor 1 and donor 2, respectively); FIGS. 23C-23D: Toledo (donor 1 and donor 2, respectively); FIGS. 23E-23F: Nalm6 (donor 1 and donor 2, respectively); FIGS. 23G-23H: Nalm6 KO (donor 1 and donor 2, respectively); FIGS. 23I-23J: K562 (donor 1 and donor 2, respectively).

FIGS. 24A-24J: Induction of T cell cytokine release by NEG258-based TBMs and BBM in various target cells. FIGS. 24A-24B: TNF-α from OC-LY-19 (donor 1 and donor 2, respectively); FIGS. 24C-24D: TNF-α from Toledo (donor 1 and donor 2, respectively); FIGS. 24E-24F: TNF-α from Nalm6 (donor 1 and donor 2, respectively); FIGS. 24G-24H: TNF-α from Nalm6 KO (donor 1 and donor 2, respectively); FIGS. 241-24J: TNF-α from K562 (donor 1 and donor 2, respectively).

FIGS. 25A-25H: Re-challenge RTCC assay with Karpas 422 and OCI-LY-19 cell lines. FIG. 25A: assay set-up. FIGS. 25B-25D: Karpas 422 (post first challenge, post second challenge, and post third challenge, respectively); FIGS. 25E-25H OCI-LY-19 (post first challenge, post second challenge, post third challenge, and post fourth challenge, respectively).

FIGS. 26A-26P: Re-challenge T cell phenotyping with Karpas 422 and OCI-LY-19 cell lines. FIGS. 26A-26H: Karpas 422 phenotyping; FIGS. 26I-26P: OCI-LY-19 phenotyping. FIGS. 26A and 261: % IL-2+CD4 T cells; FIGS. 26B and 26J: % IFNγ+CD4 T cells; FIGS. 26C and 26K: % IL-2+CD8 T cells; FIGS. 26D and 26L: % IFNγ+CD8 T cells; FIGS. 26E and 26M: CD3 young; FIGS. 26F and 26N: CD4 old; FIGS. 26G and 26O: CD8 young; FIGS. 26H and 26P: CD8 old. Lines in figures represent different T cell donors.

FIGS. 27A-27D: Ability of CD3hi TSP1 vs. CD3hi BSP1 to elicit T cell proliferation in presence of CD19+ target cells. Nalm6-luc cells were co-cultured for 72 h with sorted CD28+ or CD28⁻ CD8 T cells at an E:T ratio of 1:3 in the presence of 1 nM (FIGS. 27A-27B) or 0.1 nM (FIGS. 27C-27D) CD3hi TSP1 or CD3hi BSP1 and in presence (FIGS. 27A and 27C) or absence (FIGS. 27B and 27D) of irradiated autologous PBMCs (T cells depleted). Proliferation was measured as percentage of CFSE-diluted cells among the live cells.

FIGS. 28A-28L: Ability of CD3hi TSP1 and CD3hi BSP1 to induce T cells' cytokines production in presence of Nalm6 CD19+ target cells (E:T 1:3). FIGS. 28A-28B: median fluorescence intensity (MFI) for GzB (FIG. 28A) and IFN-γ (FIG. 28B) producing CD28⁻ and CD28⁺CD8 T cells, when co-cultured in presence of irradiated PBMCs and 1 nM CD3hi TSP1 or 1 nM CD3hi BSP1. FIGS. 28C-28D: MFI for GzB (FIG. 28C) and IFN-γ (FIG. 28D) producing CD28⁻ and CD28⁺CD8 T cells, when co-cultured in absence of irradiated PBMCs and 1 nM CD3hi TSP1 or 1 nM CD3hi BSP1. FIGS. 28E-28F: MFI for GzB (FIG. 28E) and IFN-γ (FIG. 28F) producing CD28⁻ and CD28⁺CD8 T cells, when co-cultured in presence of irradiated PBMCs and 0.1 nM CD3hi TSP1 or 0.1 nM CD3hi BSP1. FIGS. 28G-28H: MFI for GzB (FIG. 28G) and IFN-γ (FIG. 28H) producing CD28⁻ and CD28⁺CD8 T cells, when co-cultured in absence of irradiated PBMCs and 0.1 nM CD3hi TSP1 or 0.1 nM CD3hi BSP1. FIGS. 28I-28L: proportions of live T cells, when co-cultured in the presence (FIGS. 281 and 28K) or absence (FIGS. 28J and 28L) of irradiated PBMCs and 1 nM (FIGS. 281 and 28J) or 0.1 nM (FIG. 28K and FIG. 28L) CD3hi TSP1 or CD3hi BSP1.

FIGS. 29A-29I: Ability of CD3hi TSP1 vs. CD3hi BSP1 to induce changes in T cell phenotype. FIG. 29A: Representative example of CD28⁻ and CD28⁺ T cells sorted for CCR7 and CD45RO expression. FIGS. 29B-29I: distribution of different T cell populations defined according to the combined expression of the two surface markers CD45RO and CCR7 (naive, CD45RO⁻CCR7⁺; central memory (CM), CD45RO⁺CCR7⁺; effector memory (EM), CD45RO⁺CCR7⁻; and terminally differentiated (TEMRA), CD45RO⁻CCR7⁻) following 72 hour co-culture (E:T 1:3) in the presence (FIGS. 29B-29E) or absence (FIGS. 29F-29I) of PBMCs and presence of 1 nM (FIGS. 29B-29C and 29F-29G) or 0.1 nM (FIGS. 29D-29E and 29H-29I) CD3hi TSP1 or CD3hi BSP1. Data for proliferating cells (CFSE−) are shown in FIGS. 29B, 29D, 29F, and 29H. Data for non-proliferating cells (CSFE+) are shown in FIGS. 29C, 29E, 29G, and 29I. Data for CD28− cells are shown on the left side of each figure and data for CD28+ cells are shown on the right side of the figure.

FIGS. 30A-30D: Ability of CD3hi TSP1 vs. CD3hi BSP1 to elicit redirected T-cell cytotoxic activity (RTCC) against CD19+ target cells. RTCC results from Nalm6-luc cells co-cultured for 72h with sorted CD28+ or CD28− CD8 T cells at an E:T ratio of 1:3 in the presence of 1 nM (FIGS. 30A and 30C) or 0.1 nM (FIGS. 30B and 30D) of CD3hi BSP1, CD3hi TSP1, or CD3hi TSP1C and in the presence (FIGS. 30A and 30B) or absence (FIGS. 30C and 30D) of irradiated autologous PBMCs (T cells depleted). (n=3) Luminescence signal was measured at the end of the co-culture incubation. Results are expressed as fold increase vs. untreated condition, where no antibodies were added in order to evaluate the background signal given by the control antibody.

FIGS. 31A-31B: Anti-tumor activity of CD3hi TSP1 (FIG. 31A) and CD3med TSP1 (FIG. 31B) in a human PBMC adoptive transfer adaptation of the OCI-LY-19 subcutaneous tumor model.

FIGS. 32A-32B: Body weight change following treatment with CD3hi TSP1 (FIG. 32A) and CD3med TSP1 (FIG. 32B) in a human PBMC adoptive transfer adaptation of the OCI-LY-19 subcutaneous tumor model.

FIG. 33: Schematic of the humanization process of a NSG mouse.

FIGS. 34A-34B: Anti-tumor activity of CD3 TSP1, CD3hi BSP1 and CD3med TSP1 in a DLBCL subcutaneous tumor model in huCD34+ NSG mice (FIG. 34A) and body weight change following treatment with CD3 TSP1, CD3hi BSP1 and CD3med TSP1 in the DLBCL subcutaneous tumor model in huCD34+ NSG mice (FIG. 34B).

FIGS. 35A-35D: Anti-tumor activity (FIGS. 35A and 35C) and body weight response (FIG. 35B and FIG. 35D) following antibody treatment with CD3hi TSP1 (FIGS. 35A and 35B) and CD3med TSP1 (FIGS. 35C and 35D) in a OCI-LY-19 DLBCL subcutaneous tumor model in huCD34+ NSG mice.

FIGS. 36A-36C: Anti-tumor activity of CD3hi BSP1 (FIG. 36A), CD3hi TSP1 (FIG. 36B), and CD3med TSP1 (FIG. 36C) in a human PBMC adoptive transfer adaptation of the Daudi-Luc subcutaneous tumor model.

FIGS. 37A-37C: Body weight change following antibody treatment with CD3hi BSP1 (FIG. 37A), CD3hi TSP1 (FIG. 37B), or CD3med TSP1 (FIG. 37C) in a human PBMC adoptive transfer adaptation of the Daudi-Luc subcutaneous tumor model.

FIGS. 38A-38B: Shows schematic overview of a Biacore measuring cycle.

FIGS. 39A.1-39C.11: Shows representative sensorgrams and response and concentration plots. FIG. 39A.1 to FIG. 39A.11 (collectively, “FIG. 39A”) show representative sensorgrams and response plots of WT IgG1, LALAPA-IgG1, LALAGA-IgG1, LALAPG-IgG1, DAPA-IgG1, LALASKPA-IgG1, DAPASK-IgG1, GADAPA-IgG1, GADAPASK-IgG1 and DANAPA-IgG1. (Concentration range: 0.2 nM-100 nM for human FcγR1A); FIG. 39B.1 to FIG. B.11 (collectively, “FIG. 39B”) show sensorgrams and binding kinetics of WT, LALAPA-IgG1, LALAGA-IgG1, LALAPG-IgG1, DAPA-IgG1, LALASKPA-IgG1, DAPASK-IgG1, GADAPA-IgG1, GADAPASK-IgG1 and DANAPA-IgG1 towards FcgammaR3A V158 (Concentration range: 1.95 nM-1000 nM for human FcγR3A V158); FIG. 39C.1 to FIG. 39C.11 (“collectively, “FIG. 39C”) show sensorgrams and binding kinetics of WT, LALAPA-IgG1, LALAGA-IgG1, LALAPG-IgG1, DAPA-IgG1, LALASKPA-IgG1, DAPASK-IgG1, GADAPA-IgG1, GADAPASK-IgG1 and DANAPA-IgG1 towards C1q. (Concentration range: 0.49 nM-250 nM for human C1q)

FIGS. 40A-40B: FIG. 40A shows the nuclear factor of activated T-cells (NFAT) pathway activity of the wild type and mutated antibodies. FIG. 40B shows the NFAT pathway activity of the wild type and mutated antibodies, cells sensitized with addition of INFgamma.

FIGS. 41A-41E: Shows representative sensorgrams and response plots of WT, DANAPA, GADAPASK, LALA and LALASKPA variants. (Concentration range: 0.2 nM-25 nM for human FcγR1A)

FIG. 42: Shows the nuclear factor of activated T-cells (NFAT) pathway activity of the wild type and mutated antibodies.

FIGS. 43A-43B: IL-6 (FIG. 43A) and TNF-α (FIG. 43B) secretion from B-cell depleted PBMC-Karpas 422 and T-cell Karpas 422 co-cultures in the presence of CD3hi TSP1 and added B cells.

FIGS. 44A-44E: BAFF-R and CD19 expression on luciferized B-cell lymphoma cell lines measured by flow cytometry. FIG. 44A.1-44A.2: DOHH-2 Luc; FIG. 44B.1-44B.2: Karpas 422 Luc; FIG. 44C.1-44C.2: OCILY-19 Luc; FIG. 44D.1-44D.2: SU-DHL-4 Luc; FIG. 44E.1-44E.2: Toledo Luc.

FIGS. 45A-45C: Combined target cell killing from the combination of the anti-BAFFR antibody VAY736 and CD3hi TSP1 TBM in B cell depleted PBMC-Karpas 422 cell co-cultures from two donors (FIGS. 45A-45B and FIG. 45C, respectively). Dotted line in each of FIGS. 45A-45C indicates target cell killing induced by the selected concentration of CD3hi TSP1 in the absence of any VAY736 or Isotype Afuc antibody.

FIGS. 46A-46D: Tumor growth in an in vivo model of DLBCL in animals treated with vehicle (FIG. 46A), VAY736 at 5 mg/kg (FIG. 46B), VAY736 50 mg/kg (FIG. 46C) or rituximab (FIG. 46D).

### 7. DETAILED DESCRIPTION

### 7.1. Definitions

As used herein, the following terms are intended to have the following meanings:

ABM chain: Individual ABMs can exist as one (e.g., in the case of an scFv) polypeptide chain or form through the association of more than one polypeptide chains (e.g., in the case of a Fab). As used herein, the term “ABM chain” refers to all or a portion of an ABM that exists on a single polypeptide chain. The use of the term “ABM chain” is intended for convenience and descriptive purposes only and does not connote a particular configuration or method of production.

ADCC: By “ADCC” or “antibody dependent cell-mediated cytotoxicity” as used herein is meant the cell-mediated reaction where nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

ADCP: By “ADCP” or antibody dependent cell-mediated phagocytosis as used herein is meant the cell-mediated reaction where nonspecific phagocytic cells that express FcγRs recognize bound antibody on a target cell and subsequently cause phagocytosis of the target cell.

Antibody: The term “antibody” as used herein refers to a polypeptide (or set of polypeptides) of the immunoglobulin family that is capable of binding an antigen non-covalently, reversibly and specifically. For example, a naturally occurring “antibody” of the IgG type is a tetramer comprising at least two heavy (H) chains and two light (L) chains inter-connected by disulfide bonds. Each heavy chain is comprised of a heavy chain variable region (abbreviated herein as VH) and a heavy chain constant region. The heavy chain constant region is comprised of three domains, CH1, CH2 and CH3. Each light chain is comprised of a light chain variable region (abbreviated herein as VL) and a light chain constant region. The light chain constant region is comprised of one domain (abbreviated herein as CL). The VH and VL regions can be further subdivided into regions of hypervariability, termed complementarity determining regions (CDR), interspersed with regions that are more conserved, termed framework regions (FR). Each VH and VL is composed of three CDRs and four FRs arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies can mediate the binding of the immunoglobulin to host tissues or factors, including various cells of the immune system (e.g., effector cells) and the first component (C1q) of the classical complement system. The term “antibody” includes, but is not limited to, monoclonal antibodies, human antibodies, humanized antibodies, camelised antibodies, chimeric antibodies, bispecific or multispecific antibodies and anti-idiotypic (anti-Id) antibodies (including, e.g., anti-Id antibodies to antibodies of the disclosure). The antibodies can be of any isotype/class (e.g., IgG, IgE, IgM, IgD, IgA and IgY) or subclass (e.g., IgG1, IgG2, IgG3, IgG4, IgA1 and IgA2).

Both the light and heavy chains are divided into regions of structural and functional homology. The terms “constant” and “variable” are used functionally. In this regard, it will be appreciated that the variable domains of both the light (VL) and heavy (VH) chain portions determine antigen recognition and specificity. Conversely, the constant domains of the light chain (CL) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. In a wild-type antibody, at the N-terminus is a variable region and at the C-terminus is a constant region; the CH3 and CL domains actually comprise the carboxy-terminus of the heavy and light chain, respectively.

Antibody fragment: The term “antibody fragment” of an antibody as used herein refers to one or more portions of an antibody. In some embodiments, these portions are part of the contact domain(s) of an antibody. In some other embodiments, these portion(s) are antigen-binding fragments that retain the ability of binding an antigen non-covalently, reversibly and specifically, sometimes referred to herein as the “antigen-binding fragment”, “antigen-binding fragment thereof,” “antigen-binding portion”, and the like. Examples of binding fragments include, but are not limited to, single-chain Fvs (scFv), a Fab fragment, a monovalent fragment consisting of the VL, VH, CL and CH1 domains; a F(ab)2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., 1989, Nature 341:544-546), which consists of a VH domain; and an isolated complementarity determining region (CDR). Thus, the term “antibody fragment” encompasses both proteolytic fragments of antibodies (e.g., Fab and F(ab)2 fragments) and engineered proteins comprising one or more portions of an antibody (e.g., an scFv).

Antibody fragments can also be incorporated into single domain antibodies, maxibodies, minibodies, intrabodies, diabodies, triabodies, tetrabodies, v-NAR and bis-scFv (see, e.g., Hollinger and Hudson, 2005, Nature Biotechnology 23: 1126-1136). Antibody fragments can be grafted into scaffolds based on polypeptides such as Fibronectin type III (Fn3) (see U.S. Pat. No. 6,703,199, which describes fibronectin polypeptide monobodies).

Antibody fragments can be incorporated into single chain molecules comprising a pair of tandem Fv segments (for example, VH—CH1-VH—CH1) which, together with complementary light chain polypeptides (for example, VL-VC-VL-VC), form a pair of antigen-binding regions (Zapata et al., 1995, Protein Eng. 8:1057-1062; and U.S. Pat. No. 5,641,870).

Antibody Numbering System: In the present specification, the references to numbered amino acid residues in antibody domains are based on the EU numbering system unless otherwise specified (for example, in Table 1). This system was originally devised by Edelman et al., 1969, Proc. Nat'l Acad. Sci. USA 63:78-85 and is described in detail in Kabat et al., 1991, in Sequences of Proteins of Immunological Interest, US Department of Health and Human Services, NIH, USA.

Antigen-binding module: The term “antigen-binding module” or “ABM” as used herein refers to a portion of a MBM that has the ability to bind to an antigen non-covalently, reversibly and specifically. An ABM can be immunoglobulin- or non-immunoglobulin-based. As used herein, the terms “ABM1” and “CD19 ABM” (and the like) refer to an ABM that binds specifically to CD19, the terms “ABM2” and “TCR ABM” (and the like) refer to an ABM that binds specifically to a component of a TCR complex, the term “ABM3” refers to an ABM that binds specifically to CD2 or to a TAA (depending on context), the term “CD2 ABM” (and the like) refers to an ABM that binds specifically to CD2, and the term “TAA ABM” (and the like) refers to an ABM that binds specifically to a TAA. The terms ABM1, ABM2, and ABM3 are used merely for convenience and are not intended to convey any particular configuration of a MBM. In some embodiments, an ABM2 binds to CD3 (referred to herein a “CD3 ABM” or the like). Accordingly, disclosures relating to ABM2 and ABM2s are also applicable to CD3 ABMs.

Antigen-binding fragment: The term “antigen-binding fragment” of an antibody refers to a portion of an antibody that retains has the ability to bind to an antigen non-covalently, reversibly and specifically.

Antigen-binding molecule: The term “antigen-binding molecule” refers to a molecule comprising one or more antigen-binding domains, for example an antibody. The antigen-binding molecule can comprise one or more polypeptide chains, e.g., one, two, three, four or more polypeptide chains. The polypeptide chains in an antigen-binding molecule can be associated with one another directly or indirectly (for example a first polypeptide chain can be associated with a second polypeptide chain which in turn can be associated with a third polypeptide chain to form an antigen-binding molecule in which the first and second polypeptide chains are directly associated with one another, the second and third polypeptide chains are directly associated with one another, and the first and third polypeptide chains are indirectly associated with one another through the second polypeptide chain).

Associated: The term “associated” in the context of an antigen-binding molecule refers to a functional relationship between two or more polypeptide chains and/or two or more portions of a single polypeptide chain. In particular, the term “associated” means that two or more polypeptides (or portions of a single polypeptide) are associated with one another, e.g., non-covalently through molecular interactions and/or covalently through one or more disulfide bridges or chemical cross-linkages, so as to produce a functional antigen-binding molecule, e.g., a BBM or TBM in which the antigen binding domains can bind their respective targets. Examples of associations that might be present in a MBM include (but are not limited to) associations between Fc regions in an Fc domain (homodimeric or heterodimeric as described in Section 7.2.2.1.5), associations between VH and VL regions in a Fab or Fv, and associations between CH1 and CL in a Fab.

BAFF: The term “BAFF” refers to the B-cell activating factor protein. BAFF is also known as tumor necrosis factor ligand superfamily member 13B and B Lymphocyte Stimulator (BLyS). The human and murine amino acid and nucleic acid sequences can be found in a public database, such as GenBank, UniProt and Swiss-Prot. For example, an amino acid sequence of human BAFF can be found as UniProt/Swiss-Prot Accession No. Q9Y275 and a nucleotide sequences encoding human BAFF can be found at Accession Nos. NM_006573.5. BAFF is a ligand for BAFFR, and plays a role in the proliferation and differentiation of B cells.

BAFFR: The term “BAFFR” refers to the B-cell activating factor receptor protein. BAFFR is also known as TNF Receptor Superfamily Member 13C (TNFRSF13C). The human and murine amino acid and nucleic acid sequences can be found in a public database, such as GenBank, UniProt and Swiss-Prot. For example, an amino acid sequence of human BAFFR can be found as UniProt/Swiss-Prot Accession No. Q96RJ3 and a nucleotide sequences encoding human BAFFR can be found at Accession Nos. NM_052945.4. It is expressed predominantly on B-lymphocytes and on a subset of T-cells.

B cell: As used herein, the term “B cell” refers to a cell of B cell lineage, which is a type of white blood cell of the lymphocyte subtype. Examples of B cells include plasmablasts, plasma cells, lymphoplasmacytoid cells, memory B cells, follicular B cells, marginal zone B cells, B-1 cells, B-2 cells, and regulatory B cells.

B cell targeting agent: As used herein the term “B cell targeting agent” refers to an agent (e.g., a therapeutic agent) that binds to a B-cell cell surface molecule (e.g., via the antigen binding domain of an antibody) and/or depletes human B cells in vitro and/or in vivo. A B cell targeting agent that depletes B cells is referred to herein as a “B cell depleting agent.” A B cell depleting agent, e.g., a B cell depleting antibody, that depletes B cells in vitro preferably depletes B cells with an EC50 of 10 nM or less, preferably with an EC50 of 1 nM or less, more preferably with an EC50 of 100 pM, or less, as measured in a human B cell depletion assay. A B cell depleting agent that depletes B cells in vivo, e.g., in a mouse model, preferably reduces in vivo the percentage of B cells up to 70%, preferably 80% and more preferably 90% or more, as measured by fluorescence activated cell sorting (FACS) of B cells. B cell depletion assays for measuring in vitro and in vivo B cell depletion are described in WO 2010/007082, the contents of which are incorporated herein by reference in their entireties.

B cell malignancy: As used herein, a B cell malignancy refers to an uncontrolled proliferation of B cells. Examples of B cell malignancy include non-Hodgkin's lymphomas (NHL), Hodgkin's lymphomas, leukemia, and myeloma. For example, a B cell malignancy can be, but is not limited to, multiple myeloma, chronic lymphocytic leukemia (CLL)/small lymphocytic lymphoma (SLL), follicular lymphoma, mantle cell lymphoma (MCL), diffuse large B-cell lymphoma (DLBCL), marginal zone lymphomas, Burkitt lymphoma, lymphoplasmacytic lymphoma (Waldenstrom macroglobulinemia), hairy cell leukemia, splenic marginal zone B-cell lymphoma, extranodal marginal zone lymphoma (EMZL), nodal marginal zone B-cell lymphoma (NZML), and primary effusion lymphoma.

Binding Sequences: In reference to Tables 1, 9, 10, 11, 14, 15, 18, or 19 (including subparts thereof), the term “binding sequences” means an ABM having a full set of CDRs, a VH-VL pair, or an scFv set forth in that table.

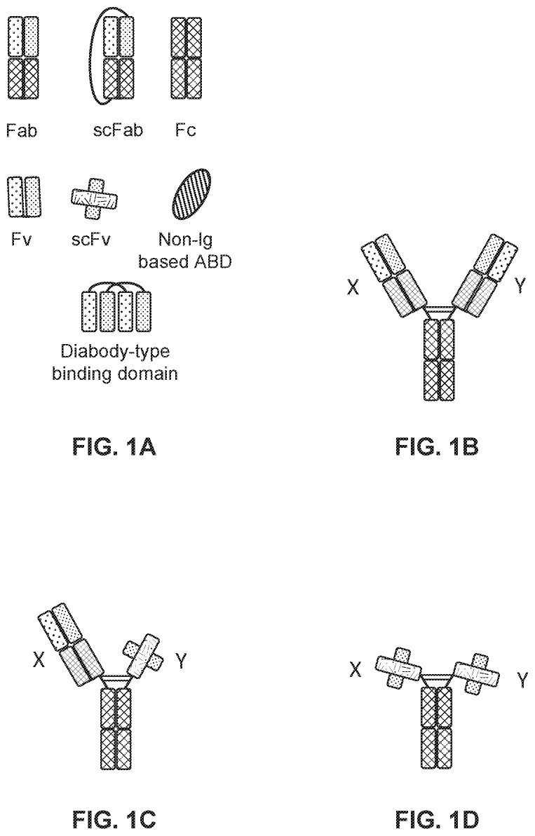

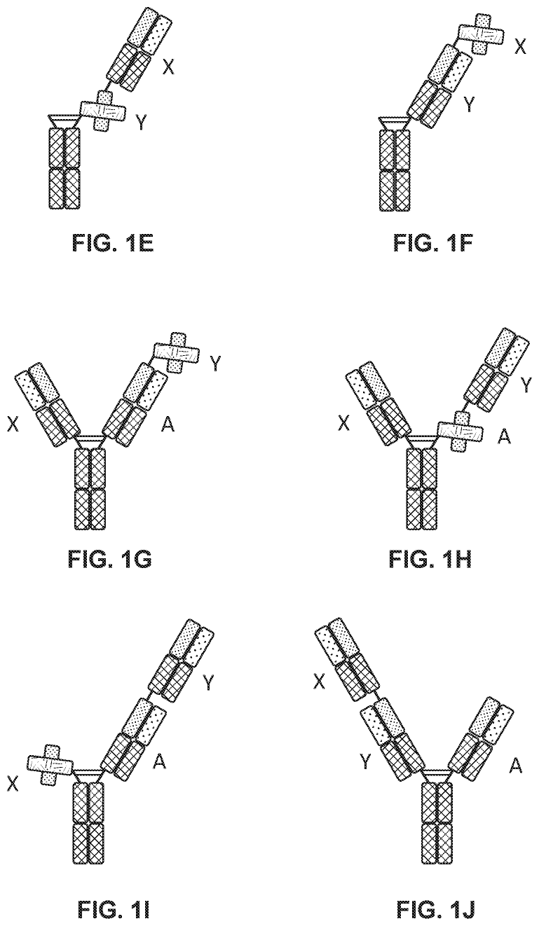

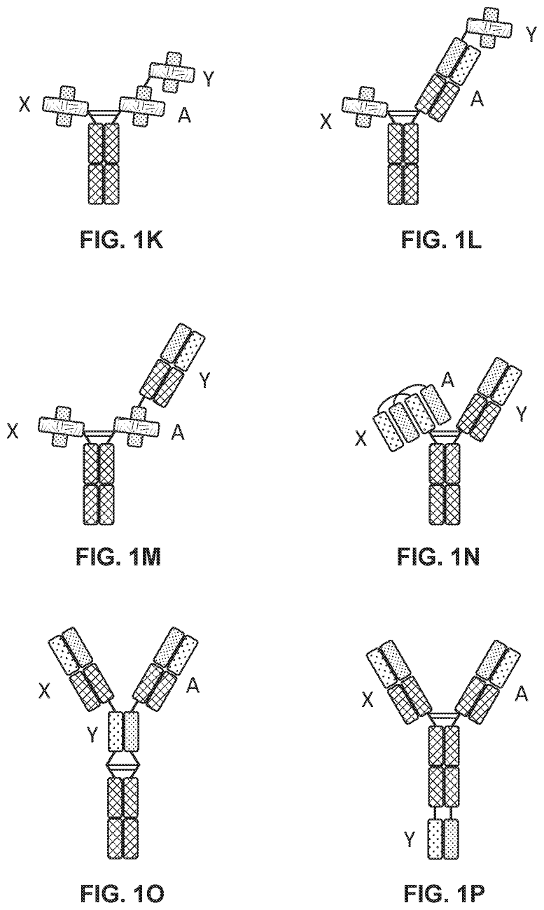

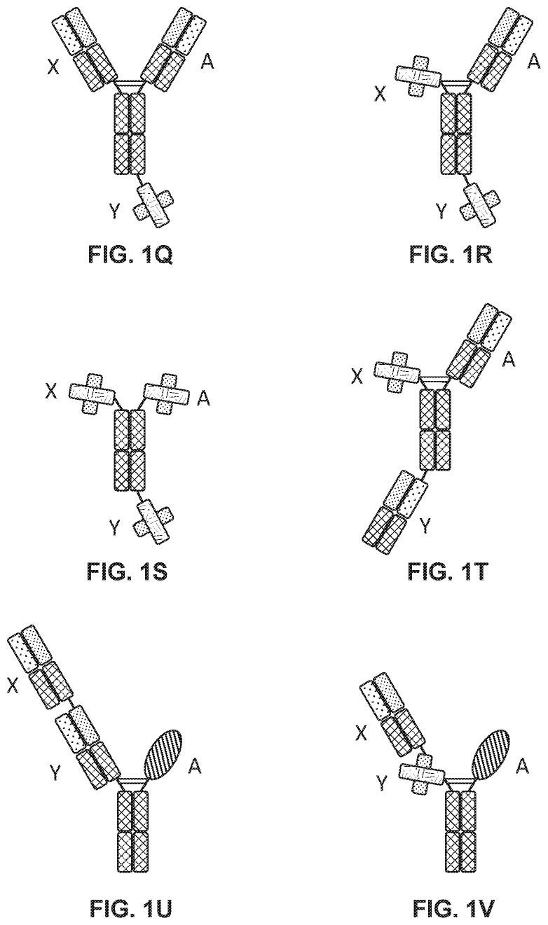

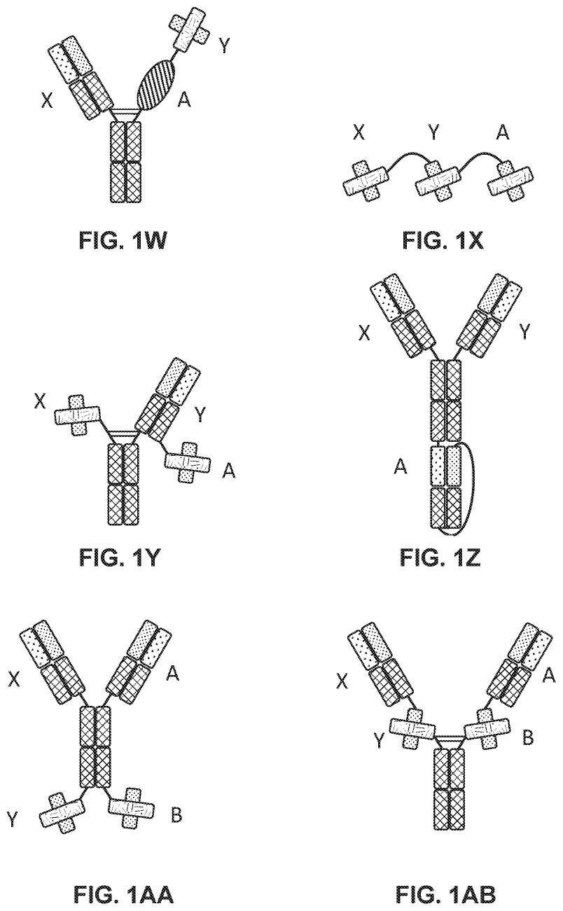

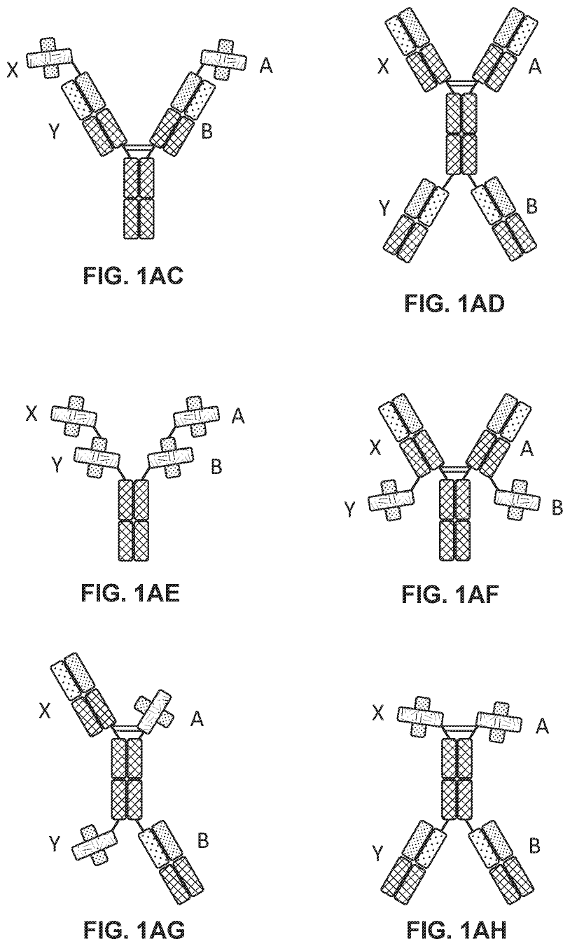

Bispecific binding molecule: The term “bispecific binding molecule” or “BBM” refers to a molecule that specifically binds to two antigens and comprises two or more ABMs. The BBMs of the disclosure comprise at least one antigen-binding domain which is specific for CD19 and at least one antigen-binding domain which is specific for a different antigen, e.g., component of a TCR complex. Representative BBMs are illustrated in FIG. 1B-1AH. BBMs can comprise one, two, three, four or even more polypeptide chains.

Bivalent: The term “bivalent” as used herein in the context of an antigen-binding molecule refers to an antigen-binding molecule that has two antigen-binding domains. The domains can be the same or different. Accordingly, a bivalent antigen-binding molecule can be monospecific or bispecific. Bivalent BBMs can comprise an ABM that specifically binds to CD19 and another ABM that binds to another antigen, e.g., a component of the TCR complex.

Cancer: The term “cancer” refers to a disease characterized by the uncontrolled (and often rapid) growth of aberrant cells. Cancer cells can spread locally or through the bloodstream and lymphatic system to other parts of the body. Examples of cancers include the B cell malignancies described herein. The term “cancerous B cell” refers to a B cell that is undergoing or has undergone uncontrolled proliferation.

CD3: The term “CD3” or “cluster of differentiation 3” refers to the cluster of differentiation 3 co-receptor of the T cell receptor. CD3 helps in activation of both cytotoxic T-cell (e.g., CD8+naïve T cells) and T helper cells (e.g., CD4+naïve T cells) and is composed of four distinct chains: one CD3γ chain (e.g., Genbank Accession Numbers NM_000073 and MP_000064 (human)), one CD36 chain (e.g., Genbank Accession Numbers NM_000732, NM_001040651, NP_00732 and NP_001035741 (human)), and two CD3E chains (e.g., Genbank Accession Numbers NM_000733 and NP_00724 (human)). The chains of CD3 are highly related cell-surface proteins of the immunoglobulin superfamily containing a single extracellular immunoglobulin domain. The CD3 molecule associates with the T-cell receptor (TCR) and ζ-chain to form the T-cell receptor (TCR) complex, which functions in generating activation signals in T lymphocytes. Unless expressly indicated otherwise, the reference to CD3 in the application can refer to the CD3 co-receptor, the CD3 co-receptor complex, or any polypeptide chain of the CD3 co-receptor complex.

CD19: The term “CD19” or “cluster of differentiation 19” refers to the Cluster of Differentiation 19 protein, which is an antigenic determinant detectable on leukemia precursor cells. The human and murine amino acid and nucleic acid sequences can be found in a public database, such as GenBank, UniProt and Swiss-Prot. For example, the amino acid sequence of human CD19 can be found as UniProt/Swiss-Prot Accession No. P15391 and the nucleotide sequence encoding of the human CD19 can be found at Accession No. NM_001178098. CD19 is expressed on most B lineage cancers, including, e.g., acute lymphoblastic leukaemia, chronic lymphocytic leukemia (CLL)/small lymphocytic lymphoma (SLL) and non-Hodgkin's lymphoma. It is also an early marker of B cell progenitors. See, e.g., Nicholson et al., 1997, Mol. Immun. 34 (16-17): 1157-1165.

Anti-CD19 Agent: The term “anti-CD19 agent” refers to an agent (e.g., a therapeutic agent) targeting CD19. Examples of anti-CD19 agents include CD19 binding molecules (including monospecific and multispecific antigen binding molecules) such as blinatumomab, NEG218-based monospecific and multispecific binding molecules described herein, NEG258-based monospecific and multispecific binding molecules described herein, and CAR T compositions such as tisagenlecleucel, axicabtagene ciloleucel, and brexucabtagene autoleucel.

Chimeric Antibody: The term “chimeric antibody” (or antigen-binding fragment thereof) is an antibody molecule (or antigen-binding fragment thereof) in which (a) the constant region, or a portion thereof, is altered, replaced or exchanged so that the antigen-binding site (variable region) is linked to a constant region of a different or altered class, effector function and/or species, or an entirely different molecule which confers new properties to the chimeric antibody, e.g., an enzyme, toxin, hormone, growth factor, drug, etc.; or (b) the variable region, or a portion thereof, is altered, replaced or exchanged with a variable region having a different or altered antigen specificity. For example, a mouse antibody can be modified by replacing its constant region with the constant region from a human immunoglobulin. Due to the replacement with a human constant region, the chimeric antibody can retain its specificity in recognizing the antigen while having reduced antigenicity in human as compared to the original mouse antibody.

Chimeric Antigen Receptor: The term “Chimeric Antigen Receptor” or alternatively a “CAR” refers to a set of polypeptides, typically two in the simplest embodiments, which when in an immune effector cell, provides the cell with specificity for a target cell, typically a cancer cell, and with intracellular signal generation. In some embodiments, a CAR comprises at least an extracellular antigen binding domain, a transmembrane domain and a cytoplasmic signaling domain (also referred to herein as “an intracellular signaling domain”) comprising a functional signaling domain derived from a stimulatory molecule and/or costimulatory molecule as defined below. The set of polypeptides can be contiguous or non-contiguous with each other. Where the polypeptides are not contiguous with one another, the set of polypeptides include a dimerization switch that, upon the presence of a dimerization molecule, can couple the polypeptides to one another, e.g., can couple an antigen binding domain to an intracellular signaling domain. CAR molecules are typically administered to a subject by way of administration of immune effector cells (e.g., T cells that are preferably autologous to the subject) engineered to express a CAR molecule.

In combination: Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. The terms “combination” and “in combination” are not limited to the administration of two or more treatments at exactly the same time, but rather it is meant that a pharmaceutical composition comprising an agent (e.g., an anti-CD19 agent or B cell targeting agent) is administered to a subject in a sequence and within a time interval such that the agent can act together with the additional therapy(ies) to provide an increased benefit than if they were administered otherwise.

Complementarity Determining Region: The terms “complementarity determining region” or “CDR,” as used herein, refer to the sequences of amino acids within antibody variable regions which confer antigen specificity and binding affinity. For example, in general, there are three CDRs in each heavy chain variable region (e.g., CDR-H1, CDR-H2, and CDR-H3) and three CDRs in each light chain variable region (CDR-L1, CDR-L2, and CDR-L3). The precise amino acid sequence boundaries of a given CDR can be determined using any of a number of well-known schemes, including those described by Kabat et al., 1991, “Sequences of Proteins of Immunological Interest,” 5th Ed. Public Health Service, National Institutes of Health, Bethesda, MD (“Kabat” numbering scheme), Al-Lazikani et al., 1997, JMB 273:927-948 (“Chothia” numbering scheme) and ImMunoGenTics (IMGT) numbering (Lefranc, 1999, The Immunologist 7:132-136; Lefranc et al., 2003, Dev. Comp. Immunol. 27:55-77 (“IMGT” numbering scheme). For example, for classic formats, under Kabat, the CDR amino acid residues in the heavy chain variable domain (VH) are numbered 31-35 (CDR-H1), 50-65 (CDR-H2), and 95-102 (CDR-H3); and the CDR amino acid residues in the light chain variable domain (VL) are numbered 24-34 (CDR-L1), 50-56 (CDR-L2), and 89-97 (CDR-L3). Under Chothia, the CDR amino acids in the VH are numbered 26-32 (CDR-H1), 52-56 (CDR-H2), and 95-102 (CDR-H3); and the amino acid residues in VL are numbered 26-32 (CDR-L1), 50-52 (CDR-L2), and 91-96 (CDR-L3). By combining the CDR definitions of both Kabat and Chothia, the CDRs consist of amino acid residues 26-35 (CDR-H1), 50-65 (CDR-H2), and 95-102 (CDR-H3) in human VH and amino acid residues 24-34 (CDR-L1), 50-56 (CDR-L2), and 89-97 (CDR-L3) in human VL. Under IMGT the CDR amino acid residues in the VH are numbered approximately 26-35 (CDR-H1), 51-57 (CDR-H2) and 93-102 (CDR-H3), and the CDR amino acid residues in the VL are numbered approximately 27-32 (CDR-L1), 50-52 (CDR-L2), and 89-97 (CDR-L3) (numbering according to “Kabat”). Under IMGT, the CDR regions of an antibody can be determined using the program IMGT/DomainGap Align.

Conservative Sequence Modifications: The term “conservative sequence modifications” refers to amino acid modifications that do not significantly affect or alter the binding characteristics of a CD19 binding molecule or a component thereof (e.g., a CD19-binding domain or an Fc region). Such conservative modifications include amino acid substitutions, additions and deletions. Modifications can be introduced into a binding molecule by standard techniques, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within a binding molecule can be replaced with other amino acid residues from the same side chain family and the altered binding molecule can be tested for, e.g., binding to target molecules and/or effective heterodimerization and/or effector function.

Diabody: The term “diabody” as used herein refers to small antibody fragments with two antigen-binding sites, typically formed by pairing of scFv chains. Each scFv comprises a heavy chain variable domain (VH) connected to a light chain variable domain (VL) in the same polypeptide chain (VH-VL, where the VH is either N-terminal or C-terminal to the VL). Unlike a typical scFv in which the VH and VL are separated by a linker that allows the VH and VL on the same polypeptide chain to pair and form an antigen-binding domain, diabodies typically comprise a linker that is too short to allow pairing between the VH and VL domains on the same chain, forcing the VH and VL domains to pair with the complementary domains of another chain and create two antigen-binding sites. Diabodies are described more fully in, for example, EP 404,097; WO 93/11161; and Hollinger et al., 1993, Proc. Natl. Acad. Sci. USA 90:6444-6448.

dsFv: The term “dsFv” refers to disulfide-stabilized Fv fragments. In a dsFv, a VH and VL are connected by an interdomain disulfide bond. To generate such molecules, one amino acid each in the framework region of in VH and VL are mutated to a cysteine, which in turn form a stable interchain disulfide bond. Typically, position 44 in the VH and position 100 in the VL are mutated to cysteines. See Brinkmann, 2010, Antibody Engineering 181-189, DOI:10.1007/978-3-642-01147-4_14. The term dsFv encompasses both what is known as a dsFv (a molecule in which the VH and VL are connected by an interchain disulfide bond but not a linker peptide) or scdsFv (a molecule in which the VH and VL are connected by a linker as well as an interchain disulfide bond).

Effector Function: The term “effector function” refers to an activity of an antibody molecule that is mediated by binding through a domain of the antibody other than the antigen-binding domain, usually mediated by binding of effector molecules. Effector function includes complement-mediated effector function, which is mediated by, for example, binding of the C1 component of the complement to the antibody. Activation of complement is important in the opsonization and lysis of cell pathogens. The activation of complement also stimulates the inflammatory response and may also be involved in autoimmune hypersensitivity. Effector function also includes Fc receptor (FcR)-mediated effector function, which can be triggered upon binding of the constant domain of an antibody to an Fc receptor (FcR). Binding of antibody to Fc receptors on cell surfaces triggers a number of important and diverse biological responses including engulfment and destruction of antibody-coated particles, clearance of immune complexes, lysis of antibody-coated target cells by killer cells (called antibody-dependent cell-mediated cytotoxicity, or ADCC), release of inflammatory mediators, placental transfer and control of immunoglobulin production. An effector function of an antibody can be altered by altering, e.g., enhancing or reducing, the affinity of the antibody for an effector molecule such as an Fc receptor or a complement component. Binding affinity will generally be varied by modifying the effector molecule binding site, and in this case it is appropriate to locate the site of interest and modify at least part of the site in a suitable way. It is also envisaged that an alteration in the binding site on the antibody for the effector molecule need not alter significantly the overall binding affinity but can alter the geometry of the interaction rendering the effector mechanism ineffective as in non-productive binding. It is further envisaged that an effector function can also be altered by modifying a site not directly involved in effector molecule binding, but otherwise involved in performance of the effector function.

Epitope: An epitope, or antigenic determinant, is a portion of an antigen recognized by an antibody or other antigen-binding moiety as described herein. An epitope can be linear or conformational.

Fab: By “Fab” or “Fab region” as used herein is meant a polypeptide region that comprises the VH, CH1, VL, and CL immunoglobulin domain. These terms can refer to this region in isolation, or this region in the context of an antigen-binding molecule of the disclosure.

Fab domains are formed by association of a CH1 domain attached to a VH domain with a CL domain attached to a VL domain. The VH domain is paired with the VL domain to constitute the Fv region, and the CH1 domain is paired with the CL domain to further stabilize the binding module. A disulfide bond between the two constant domains can further stabilize the Fab domain.

Fab regions can be produced by proteolytic cleavage of immunoglobulin molecules (e.g., using enzymes such as papain) or through recombinant expression. In native immunoglobulin molecules, Fabs are formed by association of two different polypeptide chains (e.g., VH—CH1 on one chain associates with VL-CL on the other chain). The Fab regions are typically expressed recombinantly, typically on two polypeptide chains, although single chain Fabs are also contemplated herein.

Fc domain: The term “Fc domain” refers to a pair of associated Fc regions. The two Fc regions dimerize to create the Fc domain. The two Fc regions within the Fc domain can be the same (such an Fc domain being referred to herein as an “Fc homodimer”) or different from one another (such an Fc domain being referred to herein as an “Fc heterodimer”).

Fc region: The term “Fc region” or “Fc chain” as used herein is meant the polypeptide comprising the CH2-CH3 domains of an IgG molecule, and in some cases, inclusive of the hinge. In EU numbering for human IgG1, the CH2-CH3 domain comprises amino acids 231 to 447, and the hinge is 216 to 230. Thus the definition of “Fc region” includes both amino acids 231-447 (CH2-CH3) or 216-447 (hinge-CH2-CH3), or fragments thereof. An “Fc fragment” in this context can contain fewer amino acids from either or both of the N- and C-termini but still retains the ability to form a dimer with another Fc region as can be detected using standard methods, generally based on size (e.g., non-denaturing chromatography, size exclusion chromatography). Human IgG Fc regions are of particular use in the present disclosure, and can be the Fc region from human IgG1, IgG2 or IgG4.

Fv: The term “Fv” refers to the minimum antibody fragment derivable from an immunoglobulin that contains a complete target recognition and binding site. This region consists of a dimer of one heavy and one light chain variable domain in a tight, noncovalent association (VH-VL dimer). It is in this configuration that the three CDRs of each variable domain interact to define a target binding site on the surface of the VH-VL dimer. Often, the six CDRs confer target binding specificity to the antibody. However, in some instances even a single variable domain (or half of an Fv comprising only three CDRs specific for a target) can have the ability to recognize and bind target. The reference to a VH-VL dimer herein is not intended to convey any particular configuration. By way of example and not limitation, the VH and VL can come together in any configuration described herein to form a half antibody, or can each be present on a separate half antibody and come together to form an antigen binding domain when the separate half antibodies associate, for example to form a TBM of the disclosure. When present on a single polypeptide chain (e.g., a scFv), the VH and be N-terminal or C-terminal to the VL.

Half Antibody: The term “half antibody” refers to a molecule that comprises at least one ABM or ABM chain and can associate with another molecule comprising an ABM or ABM chain through, e.g., a disulfide bridge or molecular interactions (e.g., knob-in-hole interactions between Fc heterodimers). A half antibody can be composed of one polypeptide chain or more than one polypeptide chains (e.g., the two polypeptide chains of a Fab). In an embodiment, a half-antibody comprises an Fc region.

An example of a half antibody is a molecule comprising a heavy and light chain of an antibody (e.g., an IgG antibody). Another example of a half antibody is a molecule comprising a first polypeptide comprising a VL domain and a CL domain, and a second polypeptide comprising a VH domain, a CH1 domain, a hinge domain, a CH2 domain, and a CH3 domain, where the VL and VH domains form an ABM. Yet another example of a half antibody is a polypeptide comprising an scFv domain, a CH2 domain and a CH3 domain.

A half antibody might include more than one ABM, for example a half-antibody comprising (in N- to C-terminal order) an scFv domain, a CH2 domain, a CH3 domain, and another scFv domain.

Half antibodies might also include an ABM chain that when associated with another ABM chain in another half antibody forms a complete ABM.

Thus, a MBM can comprise one, more typically two, or even more than two half antibodies, and a half antibody can comprise one or more ABMs or ABM chains.

In some MBMs, a first half antibody will associate, e.g., heterodimerize, with a second half antibody. In other MBMs, a first half antibody will be covalently linked to a second half antibody, for example through disulfide bridges or chemical crosslinking. In yet other MBMs, a first half antibody will associate with a second half antibody through both covalent attachments and non-covalent interactions, for example disulfide bridges and knob-in-hole interactions.

The term “half antibody” is intended for descriptive purposes only and does not connote a particular configuration or method of production. Descriptions of a half antibody as a “first” half antibody, a “second” half antibody, a “left” half antibody, a “right” half antibody or the like are merely for convenience and descriptive purposes.

Hexavalent: The term “hexavalent” as used herein in the context of an antigen-binding molecule (e.g., a TBM) refers to an antigen-binding molecule that has six antigen-binding domains. Hexavalent TBMs of the disclosure generally have three pairs of antigen-binding domains that each bind to the same antigen, although different configurations (e.g., three antigen-binding domains that bind to CD19, two antigen-binding domains that bind to a component of a TCR complex, and one antigen-binding domain that binds to CD2 or a TAA, or three antigen-binding domains that bind to CD19, two antigen-binding domains that bind to CD2 or a TAA, and one antigen-binding domain that binds to a component of a TCR complex) are within the scope of the disclosure. Examples of hexavalent TBMs are shown schematically in FIGS. 1U-1V.

Hole: In the context of a knob-into-hole, a “hole” refers to at least one amino acid side chain which is recessed from the interface of a first Fc chain and is therefore positionable in a compensatory “knob” on the adjacent interfacing surface of a second Fc chain so as to stabilize the Fc heterodimer, and thereby favor Fc heterodimer formation over Fc homodimer formation, for example.

Host cell or recombinant host cell: The terms “host cell” or “recombinant host cell” refer to a cell that has been genetically-engineered, e.g., through introduction of a heterologous nucleic acid. It should be understood that such terms are intended to refer not only to the particular subject cell but to the progeny of such a cell. Because certain modifications can occur in succeeding generations due to either mutation or environmental influences, such progeny may not, in fact, be identical to the parent cell, but are still included within the scope of the term “host cell” as used herein. A host cell can carry the heterologous nucleic acid transiently, e.g., on an extrachromosomal heterologous expression vector, or stably, e.g., through integration of the heterologous nucleic acid into the host cell genome. For purposes of expressing an antigen-binding molecule, a host cell can be a cell line of mammalian origin or mammalian-like characteristics, such as monkey kidney cells (COS, e.g., COS-1, COS-7), HEK293, baby hamster kidney (BHK, e.g., BHK21), Chinese hamster ovary (CHO), NSO, PerC6, BSC-1, human hepatocellular carcinoma cells (e.g., Hep G2), SP2/0, HeLa, Madin-Darby bovine kidney (MDBK), myeloma and lymphoma cells, or derivatives and/or engineered variants thereof. The engineered variants include, e.g., glycan profile modified and/or site-specific integration site derivatives.

Human Antibody: The term “human antibody” as used herein includes antibodies having variable regions in which both the framework and CDR regions are derived from sequences of human origin. Furthermore, if the antibody contains a constant region, the constant region also is derived from such human sequences, e.g., human germline sequences, or mutated versions of human germline sequences or antibody containing consensus framework sequences derived from human framework sequences analysis, for example, as described in Knappik et al., 2000, J Mol Biol 296, 57-86. The structures and locations of immunoglobulin variable domains, e.g., CDRs, can be defined using well known numbering schemes, e.g., the Kabat numbering scheme, the Chothia numbering scheme, or a combination of Kabat and Chothia (see, e.g., Lazikani et al., 1997, J. Mol. Bio. 273:927 948; Kabat et al., 1991, Sequences of Proteins of Immunological Interest, 5th edit., NIH Publication no. 91-3242 U.S. Department of Health and Human Services; Chothia et al., 1987, J. Mol. Biol. 196:901-917; Chothia et al., 1989, Nature 342:877-883).

Human antibodies can include amino acid residues not encoded by human sequences (e.g., mutations introduced by random or site-specific mutagenesis in vitro or by somatic mutation in vivo, or a conservative substitution to promote stability or manufacturing). However, the term “human antibody”, as used herein, is not intended to include antibodies in which CDR sequences derived from the germline of another mammalian species, such as a mouse, have been grafted onto human framework sequences.

Humanized: The term “humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired specificity, affinity, and capacity. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin lo sequence. The humanized antibody optionally will also comprise at least a portion of an immunoglobulin constant region (Fc), typically that of a human immunoglobulin. For further details, see Jones et al., 1986, Nature 321:522-525; Riechmann et al., 1988, Nature 332:323-329; and Presta, 1992, Curr. Op. Struct. Biol. 2:593-596. See also the following review articles and references cited therein: Vaswani and Hamilton, 1998, Ann. Allergy, Asthma & Immunol. 1:105-115; Harris, 1995, Biochem. Soc. Transactions 23:1035-1038; Hurle and Gross, 1994, Curr. Op. Biotech. 5:428-433.

Knob: In the context of a knob-into-hole, a “knob” refers to at least one amino acid side chain which projects from the interface of a first Fc chain and is therefore positionable in a compensatory “hole” in the interface with a second Fc chain so as to stabilize the Fc heterodimer, and thereby favor Fc heterodimer formation over Fc homodimer formation, for example.

Knobs and holes (or knobs-into-holes): One mechanism for Fc heterodimerization is generally referred to in the art as “knobs and holes”, or “knob-in-holes”, or “knobs-into-holes”. These terms refer to amino acid mutations that create steric influences to favor formation of Fc heterodimers over Fc homodimers, as described in, e.g., Ridgway et al., 1996, Protein Engineering 9(7):617; Atwell et al., 1997, J. Mol. Biol. 270:26; and U.S. Pat. No. 8,216,805. Knob-in-hole mutations can be combined with other strategies to improve heterodimerization, for example as described in Section 7.2.2.1.6.

Monoclonal Antibody: The term “monoclonal antibody” as used herein refers to polypeptides, including antibodies, antibody fragments, molecules (including MBMs), etc. that are derived from the same genetic source.

Monovalent: The term “monovalent” as used herein in the context of an antigen-binding molecule refers to an antigen-binding molecule that has a single antigen-binding domain.

Multispecific binding molecules: The term “multispecific binding molecules” or “MBMs” refers to molecules that specifically bind to at least two antigens and comprise two or more antigen-binding domains. The antigen-binding domains can each independently be an antibody fragment (e.g., scFv, Fab, nanobody), a ligand, or a non-antibody derived binder (e.g., fibronectin, Fynomer, DARPin).

Mutation or modification: In the context of the primary amino acid sequence of a polypeptide, the terms “modification” and “mutation” refer to an amino acid substitution, insertion, and/or deletion in the polypeptide sequence relative to a reference polypeptide. Additionally, the term “modification” further encompasses an alteration to an amino acid residue, for example by chemical conjugation (e.g., of a drug or polyethylene glycol moiety) or post-translational modification (e.g., glycosylation).

Nucleic Acid: The term “nucleic acid” is used herein interchangeably with the term “polynucleotide” and refers to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, and peptide-nucleic acids (PNAs).

Unless otherwise indicated, a particular nucleic acid sequence also implicitly encompasses conservatively modified variants thereof (e.g., degenerate codon substitutions) and complementary sequences, as well as the sequence explicitly indicated. Specifically, as detailed below, degenerate codon substitutions can be achieved by generating sequences in which the third position of one or more selected (or all) codons is substituted with mixed-base and/or deoxyinosine residues (Batzer et al., 1991, Nucleic Acid Res. 19:5081; Ohtsuka et al., 1985, J. Biol. Chem. 260:2605-2608; and Rossolini et al., 1994, Mol. Cell. Probes 8:91-98).

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of an antigen-binding molecule, separate ABMs (or chains of an ABM) can be operably linked through peptide linker sequences. In the context of a nucleic acid encoding a fusion protein, such as a polypeptide chain of an antigen-binding molecule, “operably linked” means that the two nucleic acids are joined such that the amino acid sequences encoded by the two nucleic acids remain in-frame. In the context of transcriptional regulation, the term refers to the functional relationship of a transcriptional regulatory sequence to a transcribed sequence. For example, a promoter or enhancer sequence is operably linked to a coding sequence if it stimulates or modulates the transcription of the coding sequence in an appropriate host cell or other expression system.

Pentavalent: The term “pentavalent” as used herein in the context of an antigen-binding molecule (e.g., a TBM) refers to an antigen-binding molecule that has five antigen-binding domains. Pentavalent TBMs of the disclosure generally have either (a) two pairs of antigen-binding domains that each bind to the same antigen and a single antigen-binding domain that binds to the third antigen or (b) three antigen-binding domains that bind to the same antigen and two antigen-binding domains that each bind to a separate antigen. An example of a pentavalent TBM is shown schematically in FIG. 1T.

Polypeptide and Protein: The terms “polypeptide” and “protein” are used interchangeably herein to refer to a polymer of amino acid residues. The terms encompass amino acid polymers in which one or more amino acid residue is an artificial chemical mimetic of a corresponding naturally occurring amino acid, as well as to naturally occurring amino acid polymers and non-naturally occurring amino acid polymer. Additionally, the terms encompass amino acid polymers that are derivatized, for example, by synthetic derivatization of one or more side chains or termini, glycosylation, PEGylation, circular permutation, cyclization, linkers to other molecules, fusion to proteins or protein domains, and addition of peptide tags or labels.

Recognize: The term “recognize” as used herein refers to an ABM that finds and interacts (e.g., binds) with its epitope.

Sequence identity: Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, CD. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, 1990, “Basic Local Alignment Search Tool,” J. Mol. Biol. 215:403-10, the “BLAST” algorithm, see blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters.

Optionally, the identity is determined over a region that is at least about 50 nucleotides (or, in the case of a peptide or polypeptide, at least about 10 amino acids) in length, or in some cases over a region that is 100 to 500 or 1000 or more nucleotides (or 20, 50, 200 or more amino acids) in length. In some embodiments, the identity is determined over a defined domain, e.g., the VH or VL of an antibody. Unless specified otherwise, the sequence identity between two sequences is determined over the entire length of the shorter of the two sequences.

Single Chain Fab or scFab: The terms “single chain Fab” and “scFab” mean a polypeptide comprising an antibody heavy chain variable domain (VH), an antibody constant domain 1 (CH1), an antibody light chain variable domain (VL), an antibody light chain constant domain (CL) and a linker, such that the VH and VL are in association with one another and the CH1 and CL are in association with one another. In some embodiments, the antibody domains and the linker have one of the following orders in N-terminal to C-terminal direction: a) VH—CH1-linker-VL-CL, b) VL-CL-linker-VH—CH1, c) VH-CL-linker-VL-CH1 or d) VL-CH1-linker-VH-CL. The linker can be a polypeptide of at least 30 amino acids, for example between 32 and 50 amino acids. The single chain Fabs are stabilized via the natural disulfide bond between the CL domain and the CH1 domain.

Single Chain Fv or scFv: The term “single-chain Fv” or “scFv” as used herein refers to antibody fragments that comprise the VH and VL domains of an antibody, where these domains are present in a single polypeptide chain. The Fv polypeptide can further comprise a polypeptide linker between the VH and VL domains which enables the scFv to form the desired structure for antigen-binding. For a review of scFv see Plückthun in The Pharmacology of Monoclonal Antibodies, vol. 113, Rosenburg and Moore eds., 1994, Springer-Verlag, New York, pp. 269-315.

Specifically (or selectively) binds: The term “specifically (or selectively) binds” to an antigen or an epitope refers to a binding reaction that is determinative of the presence of a cognate antigen or an epitope in a heterogeneous population of proteins and other biologics. The binding reaction can be but need not be mediated by an antibody or antibody fragment, but can also be mediated by, for example, any type of ABM described in Section 7.2.1, such as a ligand, a DARPin, etc. An ABM typically also has a dissociation rate constant (KD) (koff/kon) of less than 5×10⁻²M, less than 10⁻²M, less than 5×10⁻³M, less than 10⁻³M, less than 5×10⁻⁴M, less than 10⁻⁴M, less than 5×10⁻⁵M, less than 10⁻⁵M, less than 5×10⁻⁶M, less than 10⁻⁶M, less than 5×10⁻⁷M, less than 10⁻⁷M, less than 5×10⁻⁸M, less than 10⁻⁸M, less than 5×10⁻⁹M, or less than 10⁻⁹M, and binds to the target antigen with an affinity that is at least two-fold greater than its affinity for binding to a non-specific antigen (e.g., HSA). Binding affinity can be measured using a Biacore, SPR or BLI assay. The term “specifically binds” does not exclude cross-species reactivity. For example, an antigen-binding module (e.g., an antigen-binding fragment of an antibody) that “specifically binds” to an antigen from one species can also “specifically bind” to that antigen in one or more other species. Thus, such cross-species reactivity does not itself alter the classification of an antigen-binding module as a “specific” binder. In certain embodiments, an antigen-binding module that specifically binds to a human antigen has cross-species reactivity with one or more non-human mammalian species, e.g., a primate species (including but not limited to one or more of Macaca fascicularis, Macaca mulatta, and Macaca nemestrina) or a rodent species, e.g., Mus musculus. In other embodiments, the antigen-binding module does not have cross-species reactivity.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Tandem of VH Domains: The term “a tandem of VH domains (or VHs)” as used herein refers to a string of VH domains, consisting of multiple numbers of identical VH domains of an antibody. Each of the VH domains, except the last one at the end of the tandem, has its C-terminus connected to the N-terminus of another VH domain with or without a linker. A tandem has at least 2 VH domains, and in particular embodiments an antigen-binding molecule has 3, 4, 5, 6, 7, 8, 9, or 10 VH domains. The tandem of VH can be produced by joining the encoding nucleic acids of each VH domain in a desired order using recombinant methods with or without a linker (e.g., as described in Section 7.2.2.3) that enables them to be made as a single polypeptide chain. The N-terminus of the first VH domain in the tandem is defined as the N-terminus of the tandem, while the C-terminus of the last VH domain in the tandem is defined as the C-terminus of the tandem.

Tandem of VL Domains: The term “a tandem of VL domains (or VLs)” as used herein refers to a string of VL domains, consisting of multiple numbers of identical VL domains of an antibody. Each of the VL domains, except the last one at the end of the tandem, has its C-terminus connected to the N-terminus of another VL with or without a linker. A tandem has at least 2 VL domains, and in particular embodiments an antigen-binding molecule has 3, 4, 5, 6, 7, 8, 9, or 10 VL domains. The tandem of VL can be produced by joining the encoding nucleic acids of each VL domain in a desired order using recombinant methods with or without a linker (e.g., as described in Section 7.2.2.3) that enables them to be made as a single polypeptide chain. The N-terminus of the first VL domain in the tandem is defined as the N-terminus of the tandem, while the C-terminus of the last VL domain in the tandem is defined as the C-terminus of the tandem.

Target Antigen: By “target antigen” as used herein is meant the molecule that is bound non-covalently, reversibly and specifically by an antigen binding domain.

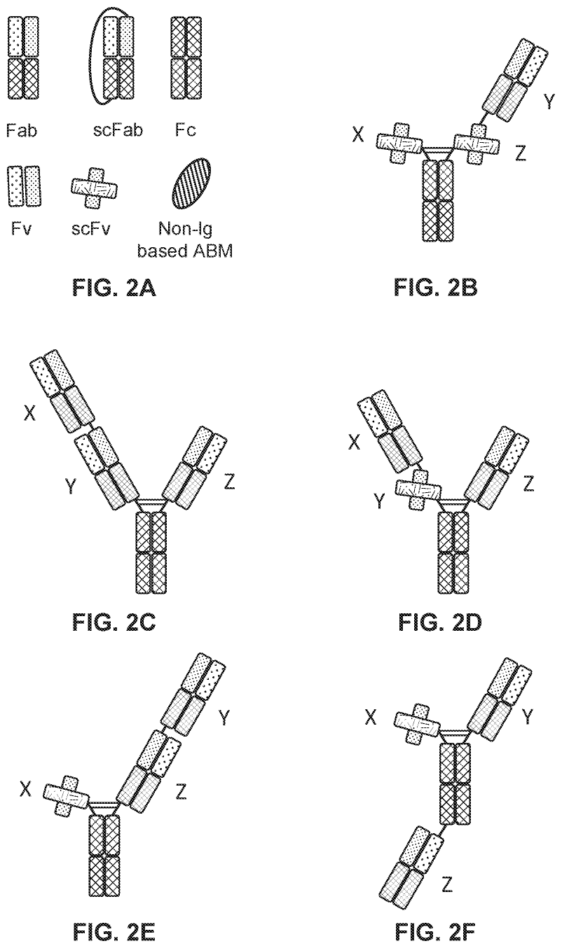

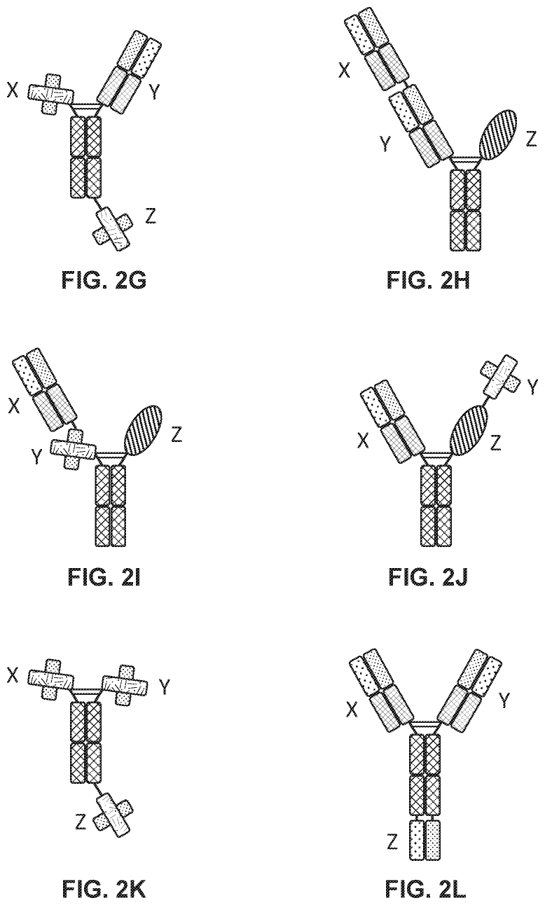

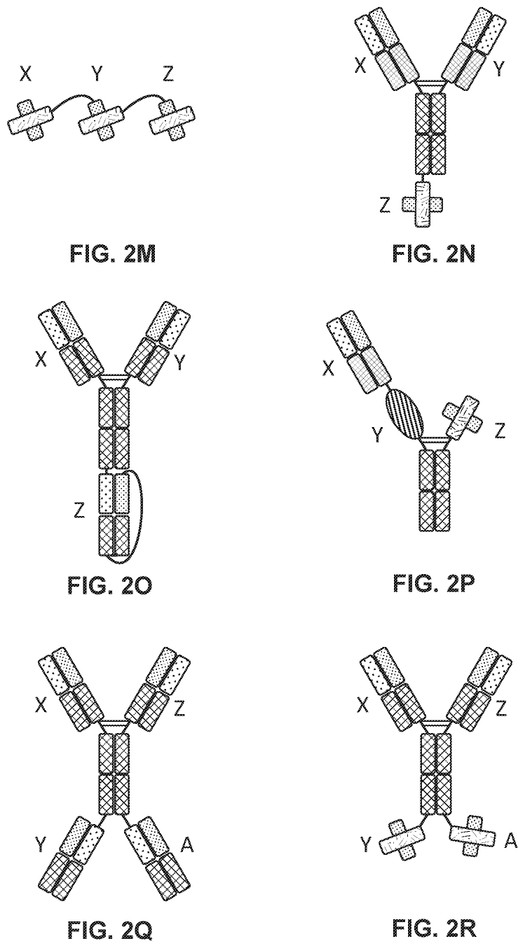

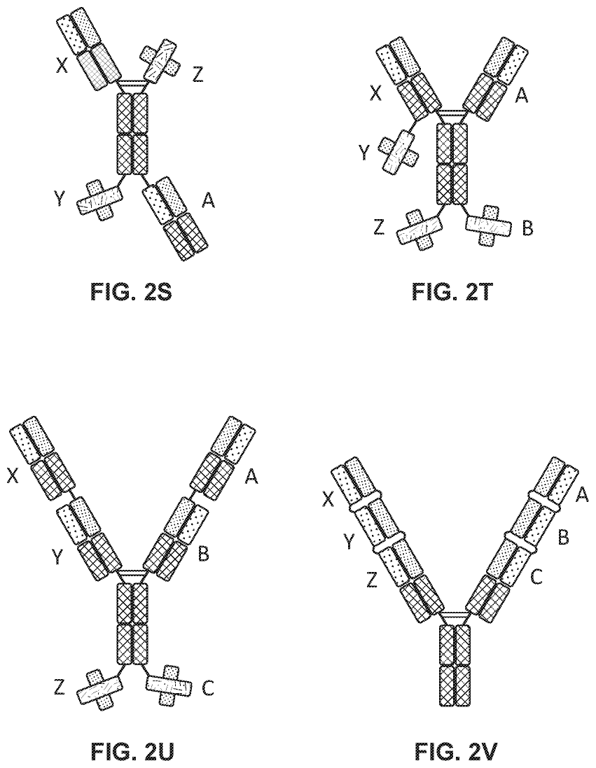

Tetravalent: The term “tetravalent” as used herein in the context of an antigen-binding molecule (e.g., a BBM or TBM) refers to an antigen-binding molecule that has four antigen-binding domains. Tetravalent TBMs of the disclosure generally have two antigen-binding domains that bind to the same antigen (e.g., CD19) and two antigen-binding domains that each bind to a separate antigen (e.g., a component of a TCR complex and either CD2 or a TAA). Examples of tetravalent BBMs are shown schematically in FIGS. 1AA-1AH and examples of tetravalent TBMs are shown schematically in FIGS. 2Q-2S.

Therapeutically effective amount: A “therapeutically effective amount” refers to an amount effective, at dosages and for periods of time necessary, to achieve a desired therapeutic result.

Treat, Treatment, Treating: As used herein, the terms “treat”, “treatment” and “treating” refer to the reduction or amelioration of the progression, severity and/or duration of a disease or disorder (e.g., a B cell malignancy), or the amelioration of the progression, severity and/or duration one or more symptoms (e.g., one or more discernible symptoms) of a disorder (e.g., CRS) resulting from the administration of one or more anti-CD19 agents. In some embodiments, the terms “treat”, “treatment” and “treating” refer to the amelioration of at least one measurable physical parameter of a disorder, such as growth of a tumor, not necessarily discernible by the patient. In other embodiments the terms “treat”, “treatment” and “treating” refer to the inhibition of the progression of a disorder, either physically by, e.g., stabilization of a discernible symptom, physiologically by, e.g., stabilization of a physical parameter, or both. In some embodiments, the terms “treat”, “treatment” and “treating” can refer to the reduction or stabilization of tumor size or cancerous cell count.

Trispecific binding molecules: The term “trispecific binding molecules” or “TBMs” refers to molecules that specifically bind to three antigens and comprise three or more antigen-binding domains. The TBMs of the disclosure comprise at least one antigen-binding domain which is specific for CD19, at least one antigen-binding domain which is specific for a component of a TCR complex, and at least one antigen-binding domain which is specific for CD2 or a TAA. The antigen-binding domains can each independently be an antibody fragment (e.g., scFv, Fab, nanobody), a ligand, or a non-antibody derived binder (e.g., fibronectin, Fynomer, DARPin). Representative TBMs are illustrated in FIG. 1. TBMs can comprise one, two, three, four or even more polypeptide chains. For example, the TBM illustrated in FIG. 1M comprises a single polypeptide chain comprising three scFvs connected by ABM linkers one a single polypeptide chain. The TBM illustrated in FIG. 1K comprises two polypeptide chains comprising three scFvs connected by, inter alia, an Fc domain. The TBM illustrated in FIG. 1J comprises three polypeptide chains forming an scFv, a ligand, and a Fab connected by, inter alia, an Fc domain. The TBM illustrated in FIG. 1C comprises four polypeptide chains forming three Fabs connected by, inter alia, an Fc domain. The TBM illustrated in FIG. 1U comprises 6 polypeptide chains forming four Fabs and two scFvs connected by, inter alia, an Fc domain.

Trivalent: The term “trivalent” as used herein in the context of an antigen-binding molecule (e.g., a MBM) refers to an antigen-binding molecule that has three antigen-binding domains. The MBMs of the disclosure are typically bispecific or trispecific. Bispecific BBMs specifically bind to CD19 and a component of a TCR complex. Trispecific TBMs specifically bind to CD19, a component of a TCR complex, and CD2 or a TAA. Accordingly, the trivalent BBMs have three antigen binding domains, two of which bind to CD19 and one of which binds to a component of the TCR, or vice versa. TBMs have three antigen-binding domains that each bind to a different antigen. Examples of trivalent BBMs are shown schematically in FIGS. 1G-1Z and examples of trivalent TBMs are shown schematically in FIGS. 2B-2V.

Tumor: The term “tumor” is used interchangeably with the term “cancer” herein, e.g., both terms encompass liquid, e.g., diffuse or circulating, tumors.

Tumor-Associated Antigen: The term “tumor-associated antigen” or “TAA” refers to a molecule (typically a protein, carbohydrate, lipid or some combination thereof) that is expressed on the surface of a cancer cell, either entirely or as a fragment (e.g., MHC/peptide), and which is useful for the preferential targeting of a pharmacological agent to the cancer cell. In some embodiments, a TAA is a marker expressed by both normal cells and cancer cells, e.g., a lineage marker, e.g., CD19 on B cells. In some embodiments, a TAA is a cell surface molecule that is overexpressed in a cancer cell in comparison to a normal cell, for instance, 1-fold over expression, 2-fold overexpression, 3-fold overexpression or more in comparison to a normal cell. In some embodiments, a TAA is a cell surface molecule that is inappropriately synthesized in the cancer cell, for instance, a molecule that contains deletions, additions or mutations in comparison to the molecule expressed on a normal cell. In some embodiments, a TAA will be expressed exclusively on the cell surface of a cancer cell, entirely or as a fragment (e.g., MHC/peptide), and not synthesized or expressed on the surface of a normal cell. Accordingly, the term “TAA” encompasses antigens that are specific to cancer cells, sometimes referred to as tumor-specific antigens (“TSAs”). Although CD19 has features of a tumor-associated antigen, the terms “tumor-associated antigen” and “TAA” are used throughout the disclosure to refer to molecules other than CD19.

Variable region: By “variable region” or “variable domain” as used herein is meant the region of an immunoglobulin that comprises one or more Ig domains substantially encoded by any of the Vκ, Vλ, and/or VH genes that make up the kappa, lambda, and heavy chain immunoglobulin genetic loci respectively, and contains the CDRs that confer antigen specificity. A “variable heavy domain” can pair with a “variable light domain” to form an antigen binding domain (“ABD”) or antigen-binding module (“ABM”). In addition, each variable domain comprises three hypervariable regions (“complementary determining regions,” “CDRs”) (CDR-H1, CDR-H2, CDR-H3 for the variable heavy domain and CDR-L1, CDR-L2, CDR-L3 for the variable light domain) and four framework (FR) regions, arranged from amino-terminus to carboxy-terminus in the following order: FR1-CDR1-FR2-CDR2-FR3-CDR3-FR4.

Vector: The term “vector” is intended to refer to a polynucleotide molecule capable of transporting another polynucleotide to which it has been linked. One type of vector is a “plasmid”, which refers to a circular double stranded DNA loop into which additional DNA segments can be ligated. Another type of vector is a viral vector, where additional DNA segments can be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operably linked. Such vectors are referred to herein as “recombinant expression vectors” (or simply, “expression vectors”). In general, expression vectors of utility in recombinant DNA techniques are often in the form of plasmids. In the present specification, “plasmid” and “vector” can be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

VH: The term “VH” refers to the variable region of an immunoglobulin heavy chain of an antibody, including the heavy chain of an Fv, scFv, dsFv or Fab.

VL: The term “VL” refers to the variable region of an immunoglobulin light chain, including the light chain of an Fv, scFv, dsFv or Fab.

VH-VL or VH-VL Pair: In reference to a VH-VL pair, whether on the same polypeptide chain or on different polypeptide chains, the terms “VH-VL” and “VH-VL pair” are used for convenience and are not intended to convey any particular orientation, unless the context dictates otherwise. Thus, a scFv comprising a “VH-VL” or “VH-VL pair” can have the VH and VL domains in any orientation, for example the VH N-terminal to the VL or the VL N-terminal to the VH.

### 7.2. Monospecific and Multispecific CD19 Binding Molecules

In some aspects, the anti-CD19 agents used in the methods and combinations of the disclosure are monospecific molecules that bind to human CD19. For example, the monospecific binding molecule can be an antibody or an antigen-binding fragment thereof (e.g., an antibody fragment, an scFv, a dsFv, a Fv, a Fab, an scFab, a (Fab′)2, or a single domain antibody (SDAB). Alternatively, the CD19 binding molecules can be multispecific molecules, for example bispecific or trispecific binding molecules.

In some embodiments, the CD19 binding molecules are chimeric or humanized monoclonal antibodies. Chimeric and/or humanized antibodies, can be engineered to minimize the immune response by a human patient to antibodies produced in non-human subjects or derived from the expression of non-human antibody genes. Chimeric antibodies comprise a non-human animal antibody variable region and a human antibody constant region. Such antibodies retain the epitope binding specificity of the original monoclonal antibody, but can be less immunogenic when administered to humans, and therefore more likely to be tolerated by the patient. For example, one or all (e.g., one, two, or three) of the variable regions of the light chain(s) and/or one or all (e.g., one, two, or three) of the variable regions the heavy chain(s) of a mouse antibody (e.g., a mouse monoclonal antibody) can each be joined to a human constant region, such as, without limitation an IgG1 human constant region. Chimeric monoclonal antibodies can be produced by known recombinant DNA techniques. For example, a gene encoding the constant region of a non-human antibody molecule can be substituted with a gene encoding a human constant region (see Robinson et al., PCT Patent Publication PCT/US86/02269; Akira, et al., European Patent Application 184,187; or Taniguchi, M., European Patent Application 171,496). In addition, other suitable techniques that can be used to generate chimeric antibodies are described, for example, in U.S. Pat. Nos. 4,816,567; 4,978,775; 4,975,369; and 4,816,397.

Chimeric or humanized antibodies and antigen binding fragments thereof can be prepared based on the sequence of a murine monoclonal antibody. DNA encoding the heavy and light chain immunoglobulins can be obtained from a murine hybridoma of interest and engineered to contain non-murine (e.g., human) immunoglobulin sequences using standard molecular biology techniques. For example, to create a chimeric antibody, the murine variable regions can be linked to human constant regions using known methods (see e.g., U.S. Pat. No. 4,816,567 to Cabilly et al.). To create a humanized antibody, the murine CDR regions can be inserted into a human framework using known methods. See e.g., U.S. Pat. No. 5,225,539 to Winter, and U.S. Pat. Nos. 5,530,101; 5,585,089; 5,693,762 and 6,180,370 to Queen et al.

A humanized antibody can be produced using a variety of known techniques, including but not limited to, CDR-grafting (see, e.g., European Patent No. EP 239,400; International Publication No. WO 91/09967; and U.S. Pat. Nos. 5,225,539, 5,530,101, and 5,585,089), veneering or resurfacing (see, e.g., European Patent Nos. EP 592,106 and EP 519,596; Padlan, 1991, Molecular Immunology, 28(4/5):489-498; Studnicka et al., 1994, Protein Engineering, 7(6):805-814; and Roguska et al., 1994, PNAS, 91:969-973), chain shuffling (see, e.g., U.S. Pat. No. 5,565,332), and techniques disclosed in, e.g., U.S. Patent Application Publication No. US2005/0042664, U.S. Patent Application Publication No. US2005/0048617, U.S. Pat. Nos. 6,407,213, 5,766,886, International Publication No. WO 9317105, Tan et al., J. Immunol., 169:1119-25 (2002), Caldas et al., Protein Eng., 13(5):353-60 (2000), Morea et al., Methods, 20(3):267-79 (2000), Baca et al., J. Biol. Chem., 272(16):10678-84 (1997), Roguska et al., Protein Eng., 9(10):895-904 (1996), Couto et al., Cancer Res., 55 (23 Supp):5973s-5977s (1995), Couto et al., Cancer Res., 55(8):1717-22 (1995), Sandhu J S, Gene, 150(2):409-10 (1994), and Pedersen et al., J. Mol. Biol., 235(3):959-73 (1994). Often, framework residues in the framework regions will be substituted with the corresponding residue from the CDR donor antibody to alter, for example improve, antigen binding. These framework substitutions, e.g., conservative substitutions are identified by known methods, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature, 332:323).

As provided herein, humanized antibodies or antibody fragments can comprise one or more CDRs from nonhuman immunoglobulin molecules and framework regions where the amino acid residues comprising the framework are derived completely or mostly from human germline. Multiple techniques for humanization of antibodies or antibody fragments are well-known and can essentially be performed following the method of Winter and co-workers (Jones et al., Nature, 321:522-525 (1986); Riechmann et al., Nature, 332:323-327 (1988); Verhoeyen et al., Science, 239:1534-1536 (1988)), by substituting rodent CDRs or CDR sequences for the corresponding sequences of a human antibody, i.e., CDR-grafting (EP 239,400; PCT Publication No. WO 91/09967; and U.S. Pat. Nos. 4,816,567; 6,331,415; 5,225,539; 5,530,101; 5,585,089; 6,548,640). In such humanized antibodies and antibody fragments, substantially less than an intact human variable domain has been substituted by the corresponding sequence from a nonhuman species. Humanized antibodies are often human antibodies in which some CDR residues and possibly some framework (FR) residues are substituted by residues from analogous sites in rodent antibodies. Humanization of antibodies and antibody fragments can also be achieved by veneering or resurfacing (EP 592,106; EP 519,596; Padlan, 1991, Molecular Immunology, 28(4/5):489-498; Studnicka et al., Protein Engineering, 7(6):805-814 (1994); and Roguska et al., PNAS, 91:969-973 (1994)) or chain shuffling (U.S. Pat. No. 5,565,332).

The choice of human variable domains, both light and heavy, to be used in making the humanized antibodies is to reduce antigenicity. According to the so-called “best-fit” method, the sequence of the variable domain of a rodent antibody is screened against the entire library of known human variable-domain sequences. The human sequence which is closest to that of the rodent is then accepted as the human framework (FR) for the humanized antibody (Sims et al., J. Immunol., 151:2296 (1993); Chothia et al., J. Mol. Biol., 196:901 (1987)). Another method uses a particular framework derived from the consensus sequence of all human antibodies of a particular subgroup of light or heavy chains. The same framework can be used for several different humanized antibodies (see, e.g., Nicholson et al. Mol. Immun. 34 (16-17): 1157-1165 (1997); Carter et al., Proc. Natl. Acad. Sci. USA, 89:4285 (1992); Presta et al., J. Immunol., 151:2623 (1993). In some embodiments, the framework region, e.g., all four framework regions, of the heavy chain variable region are derived from a VH4_4-59 germline sequence. In one embodiment, the framework region can comprise, one, two, three, four or five modifications, e.g., substitutions, e.g., conservative substitutions, e.g., from the amino acid at the corresponding murine sequence. In one embodiment, the framework region, e.g., all four framework regions of the light chain variable region are derived from a VK3_1.25 germline sequence. In one embodiment, the framework region can comprise, one, two, three, four or five modifications, e.g., substitutions, e.g., conservative substitutions, e.g., from the amino acid at the corresponding murine sequence.

In certain embodiments, the CD19 binding molecules comprise a heavy chain variable region from a particular germline heavy chain immunoglobulin gene and/or a light chain variable region from a particular germline light chain immunoglobulin gene. For example, such antibodies can comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence can contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody can be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pl and ablation variants herein; that is, the number of variants is generally low, prior to the introduction of the variants of the disclosure). In certain cases, the humanized antibody can display no more than 5, or even no more than 4, 3, 2, or 1 amino acid difference from the amino acid sequence encoded by the germline immunoglobulin gene (again, prior to the introduction of any skew, pl and ablation variants herein; that is, the number of variants is generally low, prior to the introduction of the variants of the disclosure).

In one embodiment, the parent antibody has been affinity matured. Structure-based methods can be employed for humanization and affinity maturation, for example as described in U.S. Ser. No. 11/004,590. Selection based methods can be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759. Other humanization methods can involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084.

In some embodiments, the CD19 binding molecule comprises an ABM which is a Fab. Fab domains can be produced by proteolytic cleavage of immunoglobulin molecules, using enzymes such as papain, or through recombinant expression. Fab domains typically comprise a CH1 domain attached to a VH domain which pairs with a CL domain attached to a VL domain. In a wild-type immunoglobulin, the VH domain is paired with the VL domain to constitute the Fv region, and the CH1 domain is paired with the CL domain to further stabilize the binding module. A disulfide bond between the two constant domains can further stabilize the Fab domain.

In some embodiments, the CD19 binding molecule comprises an ABM which is a scFab. In an embodiment, the antibody domains and the linker in the scFab fragment have one of the following orders in N-terminal to C-terminal direction: a) VH—CH1-linker-VL-CL, or b) VL-CL-linker-VH—CH1. In some cases, VL-CL-linker-VH—CH1 is used.

In another embodiment, the antibody domains and the linker in the scFab fragment have one of the following orders in N-terminal to C-terminal direction: a) VH-CL-linker-VL-CH1 or b) VL-CH1-linker-VH-CL.

Optionally in the scFab fragment, additionally to the natural disulfide bond between the CL-domain and the CH1 domain, also the antibody heavy chain variable domain (VH) and the antibody light chain variable domain (VL) are disulfide stabilized by introduction of a disulfide bond between the following positions: i) heavy chain variable domain position 44 to light chain variable domain position 100, ii) heavy chain variable domain position 105 to light chain variable domain position 43, or iii) heavy chain variable domain position 101 to light chain variable domain position 100 (numbering according to EU index of Kabat).

Such further disulfide stabilization of scFab fragments is achieved by the introduction of a disulfide bond between the variable domains VH and VL of the single chain Fab fragments. Techniques to introduce unnatural disulfide bridges for stabilization for a single chain Fv are described e.g. in WO 94/029350, Rajagopal et al., 1997, Prot. Engin. 10:1453-59; Kobayashi et al., 1998, Nuclear Medicine & Biology, 25:387-393; and Schmidt, et al., 1999, Oncogene 18:1711-1721. In one embodiment, the optional disulfide bond between the variable domains of the scFab fragments is between heavy chain variable domain position 44 and light chain variable domain position 100. In one embodiment, the optional disulfide bond between the variable domains of the scFab fragments is between heavy chain variable domain position 105 and light chain variable domain position 43 (numbering according to EU index of Kabat).

In some embodiments, the CD19 binding molecule comprises an ABM which is a scFv. Single chain Fv antibody fragments comprise the VH and VL domains of an antibody in a single polypeptide chain, are capable of being expressed as a single chain polypeptide, and retain the specificity of the intact antibody from which it is derived. Generally, the scFv polypeptide further comprises a polypeptide linker between the VH and VL domain that enables the scFv to form the desired structure for target binding. Examples of linkers suitable for connecting the VH and VL chains of an scFV are the ABM linkers identified in Section 7.2.2.3, for example any of the linkers designated L1 through L58.

Unless specified, as used herein an scFv can have the VL and VH variable regions in either order, e.g., with respect to the N-terminal and C-terminal ends of the polypeptide, the scFv can comprise VL-linker-VH or can comprise VH-linker-VL.

To create an scFv-encoding nucleic acid, the VH and VL-encoding DNA fragments are operably linked to another fragment encoding a linker, e.g., encoding any of the linkers described in Section 7.2.2.3 (such as the amino acid sequence (Gly4″Ser)3 (SEQ ID NO: 1174)), such that the VH and VL sequences can be expressed as a contiguous single-chain protein, with the VL and VH regions joined by the flexible linker (see e.g., Bird et al., 1988, Science 242:423-426; Huston et al., 1988, Proc. Natl. Acad. Sci. USA 85:5879-5883; McCafferty et al., 1990, Nature 348:552-554).

CD19 binding molecules can also comprise an ABM which is a Fv, a dsFv, a (Fab′)2, a single domain antibody (SDAB), a VH or VL domain, or a camelid VHH domain (also called a nanobody).

CD19 binding molecules can comprise a single domain antibody composed of a single VH or VL domain which exhibits sufficient affinity to CD19. In an embodiment, the single domain antibody is a camelid VHH domain (see, e.g., Riechmann, 1999, Journal of Immunological Methods 231:25-38; WO 94/04678).

Tables 1A to 1C (collectively “Table 1”) list the sequences of exemplary CD19 binding sequences that can be included in CD19 binding molecules.

The sequences set forth in Table 1A are based on the CD19 antibody NEG258.

TABLE 1A
NEG258-Based Binder Sequences

| | | | SEQ |
|---|---|---|---|
| Chain | Portion | Sequence | ID NO: |
| NEG258_VH | CDR-H1 | GYTFTTYWIQ | 1 |
| | (Combined) | | |
| | CDR-H2 | AVYPGDADTRYTQKFQG | 2 |
| | (Combined) | | |
| | CDR-H3 | DAGLEYYALDY | 3 |
| | (Combined) | | |
| | CDR-H1 (Kabat) | TYWIQ | 4 |
| | CDR-H2 (Kabat) | AVYPGDADTRYTQKFQG | 5 |
| | CDR-H3 (Kabat) | DAGLEYYALDY | 6 |
| | CDR-H1 (Chothia) | GYTFTTY | 7 |
| | CDR-H2 (Chothia) | YPGDAD | 8 |
| | CDR-H3 (Chothia) | DAGLEYYALDY | 9 |
| | CDR-H1 (IMGT) | GYTFTTYW | 10 |
| | CDR-H2 (IMGT) | VYPGDADT | 11 |
| | CDR-H3 (IMGT) | GRDAGLEYYALDY | 12 |
| | VH | QVQLVQSGAEVKKPGASVKVSCKASGYTFTTYW | 13 |
| | | IQWVRQAPGQRLEWMGAVYPGDADTRYTQKFQG | |
| | | RVTLTADRSASTAYMELSSLRSEDTAVYYCGRD | |
| | | AGLEYYALDYWGQGTLVTVSS | |
| NEG258_VL | CDR-L1 | RASQDVGTAVA | 14 |
| | (Combined) | | |
| | CDR-L2 | WASTRHT | 15 |
| | (Combined) | | |
| | CDR-L3 | QQYANFPLYT | 16 |
| | (Combined) | | |
| | CDR-L1 (Kabat) | RASQDVGTAVA | 17 |
| | CDR-L2 (Kabat) | WASTRHT | 18 |
| | CDR-L3 (Kabat) | QQYANFPLYT | 19 |
| | CDR-L1 (Chothia) | SQDVGTA | 20 |
| | CDR-L2 (Chothia) | WAS | 21 |
| | CDR-L3 (Chothia) | YANFPLY | 22 |
| | CDR-L1 (IMGT) | QDVGTA | 23 |
| | CDR-L2 (IMGT) | WAS | 24 |
| | CDR-L3 (IMGT) | QQYANFPLYT | 25 |
| | VL | EIVMTQSPATLSVSPGERATLSCRASQDVGTAV | 26 |
| | | AWYQQKPGQAPRLLIYWASTRHTGIPARFSGSG | |
| | | SGTEFTLTISSLQSEDFAVYFCQQYANFPLYTF | |
| | | GQGTKLEIK | |

In some embodiments, a CD19 binding molecule comprises CDR-L1, CDR-L-2, CDR-L-3, CDR-H1, CDR-H-2 and CDR-H-3 sequences of NEG258 as set forth in Table 1A. The CDR-L1, CDR-L-2, CDR-L-3, CDR-H1, CDR-H-2 and CDR-H-3 sequences can be as defined by Kabat (SEQ ID NOs:17-19 and 4-6, respectively), Chothia (SEQ ID NOs:20-22 and 7-9, respectively), or IMGT (SEQ ID NOs& 23-25 and 10-12, respectively), or the combined Chothia and Kabat CDR-L1, CDR-L-2, CDR-L-3, CDR-H1, CDR-H-2 and CDR-H-3 sequences (SEQ ID NOs:14-16 and 1-3, respectively). The CD19 binding molecule can also comprise a light chain variable sequence (SEQ ID NO:26) and/or heavy chain variable sequence (SEQ ID NO:13) of the anti-0019 antibody NEG258 as set forth in Table 1A.

The sequences set forth in Table 1B are based on the CD19 antibody NEG218.

TABLE 1B
NEG218-Based Sequences

| Chain | Portion | Sequence | SEQ ID NO: |
|---|---|---|---|
| NEG218_VH | CDR-H1 | GYSFTNYWMN | 27 |
| | (Combined) | | |
| | CDR-H2 | MIHPSDSEIRLNQKFQG | 28 |
| | (Combined) | | |
| | CDR-H3 | WYYLSSPMDY | 29 |
| | (Combined) | | |
| | CDR-H1 (Kabat) | NYWMN | 30 |
| | CDR-H2 (Kabat) | MIHPSDSEIRLNQKFQG | 31 |
| | CDR-H3 (Kabat) | WYYLSSPMDY | 32 |
| | CDR-H1 (Chothia) | GYSFTNY | 33 |
| | CDR-H2 (Chothia) | HPSDSE | 34 |
| | CDR-H3 (Chothia) | WYYLSSPMDY | 35 |
| | CDR-H1 (IMGT) | GYSFTNYW | 36 |
| | CDR-H2 (IMGT) | IHPSDSEI | 37 |
| | CDR-H3 (IMGT) | SRWYYLSSPMDY | 38 |
| | VH | EVQLVQSGAEVKKPGESLKISCKASGYSFTNYWMNWVRQ | 39 |
| | | MPGKGLEWMGMIHPSDSEIRLNQKFQGQVTLSVDKSIGTA | |
| | | YMQWSSLKASDTAMYYCSRWYYLSSPMDYWGQGTTVTV | |
| | | SS | |
| NEG218_VL | CDR-L1 | RASQDVGTAVA | 40 |
| | (Combined) | | |
| | CDR-L2 | WASTRHT | 41 |
| | (Combined) | | |
| | CDR-L3 | QQYSSYPYT | 42 |
| | (Combined) | | |
| | CDR-L1 (Kabat) | RASQDVGTAVA | 43 |
| | CDR-L2 (Kabat) | WASTRHT | 44 |
| | CDR-L3 (Kabat) | QQYSSYPYT | 45 |
| | CDR-L1 (Chothia) | SQDVGTA | 46 |
| | CDR-L2 (Chothia) | WAS | 47 |
| | CDR-L3 (Chothia) | YSSYPY | 48 |
| | CDR-L1 (IMGT) | QDVGTA | 49 |
| | CDR-L2 (IMGT) | WAS | 50 |
| | CDR-L3 (IMGT) | QQYSSYPYT | 51 |
| | VL | EIVMTQSPATLSVSPGERATLSCRASQDVGTAVAWYQQKP | 52 |
| | | GQAPRLLIYWASTRHTGIPARFSGSGSGTEFTLTISSLQSE | |
| | | DFAVYFCQQYSSYPYTFGQGTKLEIK | |

In some embodiments, a CD19 binding molecule comprises CDR-L1, CDR-L2, CDR-L3, CDR-H1, CDR-H2 and CDR-H3 sequences of NEG218 as set forth in Table 214. The CDR-L, CDR-L2, CDR-H3, CDR-H1, CDR-H2 and CDR-H3 sequences can be as defined by Kabat (SEQ ID NOs:43-45 and 30-32, respectively), Chothia (SEQ ID NOs:46-48 and 33-35, respectively), or IMGT (SEQ ID NOs:49-51 and 36-38, respectively), or the combined Chothia and Kabat CDR-L1, CDR-2, CDR-3, CR-H1, CDR-H2 and CDR-H3 sequences (SEQ ID NOs:40-42 and 27-29, respectively). The CD19 binding molecule can also comprise a light chain variable sequence (SEQ ID NO:52) and/or heavy chain variable sequence (SEQ ID NO:39) of the anti-CD9 antibody NEG218 as set forth in Table 1B.

Exemplary CD19 binding molecules having CDR sequences described in Table 1A and Table 1 B are provided in Table 20A-1 to Table 200.

Further exemplary CDR and variable domain sequences that can be incorporated into a CD19 binding molecule are set forth in Table 1C.

TABLE 1C
CD19 Binders

| | | | SEQ |
|---|---|---|---|
| | | | ID |
| Name | Domain | Sequence | NO: |
| CD19-H1 | CDR-H1 | DYGVS | 214 |
| CD19-H2A | CDR-H2 | VIWGSETTYYNSALKS | 114 |
| CD19-H2B | CDR-H2 | VIWGSETTYYSSSLKS | 115 |
| CD19-H2C | CDR-H2 | VIWGSETTYYQSSLKS | 116 |
| CD19-H2D | CDR-H2 | VIWGSETTYYNSSLKS | 117 |
| CD19-H3 | CDR-H3 | HYYYGGSYAMDY | 118 |
| CD19-L1 | CDR-L1 | RASQDISKYLN | 119 |
| CD19-L2 | CDR-L2 | HTSRLHS | 120 |
| CD19-L3 | CDR-L3 | QQGNTLPYT | 121 |
| CD19-VHA | VH | EVKLQESGPGLVAPSQSLSVTCTVSGVSLPDYGVSWI | 218 |
| | | RQPPRKGLEWLGVIWGSETTYYNSALKSRLTIIKDNSK | |
| | | SQVFLKMNSLQTDDTAIYYCAKHYYYGGSYAMDYWG | |
| | | QGTSVTVSS | |
| CD19-VHB | VH | QVQLQESGPGLVKPSETLSLTCTVSGVSLPDYGVSWI | 219 |
| | | RQPPGKGLEWIGVIWGSETTYYSSSLKSRVTISKDNSK | |
| | | NQVSLKLSSVTAADTAVYYCAKHYYYGGSYAMDYWG | |
| | | QGTLVTVSS | |
| CD19-VHC | VH | QVQLQESGPGLVKPSETLSLTCTVSGVSLPDYGVSWI | 220 |
| | | RQPPGKGLEWIGVIWGSETTYYQSSLKSRVTISKDNSK | |
| | | NQVSLKLSSVTAADTAVYYCAKHYYYGGSYAMDYWG | |
| | | QGTLVTVSS | |
| CD19-VHD | VH | QVQLQESGPGLVKPSETLSLTCTVSGVSLPDYGVSWI | 221 |
| | | RQPPGKGLEWIGVIWGSETTYYNSSLKSRVTISKDNSK | |
| | | NQVSLKLSSVTAADTAVYYCAKHYYYGGSYAMDYWG | |
| | | QGTLVTVSS | |
| CD19-VLA | VL | DIQMTQTTSSLSASLGDRVTISCRASQDISKYLNWYQQ | 222 |
| | | KPDGTVKLLIYHTSRLHSGVPSRFSGSGSGTDYSLTIS | |
| | | NLEQEDIATYFCQQGNTLPYTFGGGTKLEIT | |
| CD19-VLB | VL | EIVMTQSPATLSLSPGERATLSCRASQDISKYLNWYQQ | 223 |
| | | KPGQAPRLLIYHTSRLHSGIPARFSGSGSGTDYTLTISS | |
| | | LQPEDFAVYFCQQGNTLPYTFGQGTKLEIK | |
| CD19-scFv1 | scFv | EIVMTQSPATLSLSPGERATLSCRASQDISKYLNWYQQ | 96 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVS | |
| | | LPDYGVSWIRQPPGKGLEWIGVIWGSETTYYSSSLKS | |
| | | RVTISKDNSKNQVSLKLSSVTAADTAVYYCAKHYYYGG | |
| | | SYAMDYWGQGTLVTVSS | |
| CD19-scFv2 | scFv | EIVMTQSPATLSLSPGERATLSCRASQDISKYLNWYQQ | 97 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVS | |
| | | LPDYGVSWIRQPPGKGLEWIGVIWGSETTYYQSSLKS | |
| | | RVTISKDNSKNQVSLKLSSVTAADTAVYYCAKHYYYGG | |
| | | SYAMDYWGQGTLVTVSS | |
| CD19-scFv3 | scFv | QVQLQESGPGLVKPSETLSLTCTVSGVSLPDYGVSWI | 98 |
| | | RQPPGKGLEWIGVIWGSETTYYSSSLKSRVTISKDNSK | |
| | | NQVSLKLSSVTAADTAVYYCAKHYYYGGSYAMDYWG | |
| | | QGTLVTVSSGGGGSGGGGSGGGGSEIVMTQSPATLS | |
| | | LSPGERATLSCRASQDISKYLNWYQQKPGQAPRLLIYH | |
| | | TSRLHSGIPARFSGSGSGTDYTLTISSLQPEDFAVYFC | |
| | | QQGNTLPYTFGQGTKLEIK | |
| CD19-scFv4 | scFv | QVQLQESGPGLVKPSETLSLTCTVSGVSLPDYGVSWI | 99 |
| | | RQPPGKGLEWIGVIWGSETTYYQSSLKSRVTISKDNSK | |
| | | NQVSLKLSSVTAADTAVYYCAKHYYYGGSYAMDYWG | |
| | | QGTLVTVSSGGGGSGGGGSGGGGSEIVMTQSPATLS | |
| | | LSPGERATLSCRASQDISKYLNWYQQKPGQAPRLLIYH | |
| | | TSRLHSGIPARFSGSGSGTDYTLTISSLQPEDFAVYFC | |
| | | QQGNTLPYTFGQGTKLEIK | |
| CD19-scFv5 | scFv | EIVMTQSPATLSLSPGERATLSCRASQDISKYLNWYQQ | 100 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYS | |
| | | SSLKSRVTISKDNSKNQVSLKLSSVTAADTAVYYCAKH | |
| | | YYYGGSYAMDYWGQGTLVTVSS | |
| CD19-scFv6 | scFv | EIVMTQSPATLSLSPGERATLSCRASQDISKYLNWYQQ | 101 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYQ | |
| | | SSLKSRVTISKDNSKNQVSLKLSSVTAADTAVYYCAKH | |
| | | YYYGGSYAMDYWGQGTLVTVSS | |
| CD19-scFv7 | scFv | QVQLQESGPGLVKPSETLSLTCTVSGVSLPDYGVSWI | 102 |
| | | RQPPGKGLEWIGVIWGSETTYYSSSLKSRVTISKDNSK | |
| | | NQVSLKLSSVTAADTAVYYCAKHYYYGGSYAMDYWG | |
| | | QGTLVTVSSGGGGSGGGGSGGGGSGGGGSEIVMTQ | |
| | | SPATLSLSPGERATLSCRASQDISKYLNWYQQKPGQA | |
| | | PRLLIYHTSRLHSGIPARFSGSGSGTDYTLTISSLQPED | |
| | | FAVYFCQQGNTLPYTFGQGTKLEIK | |
| CD19-scFv8 | scFv | QVQLQESGPGLVKPSETLSLTCTVSGVSLPDYGVSWI | 103 |
| | | RQPPGKGLEWIGVIWGSETTYYQSSLKSRVTISKDNSK | |
| | | NQVSLKLSSVTAADTAVYYCAKHYYYGGSYAMDYWG | |
| | | QGTLVTVSSGGGGSGGGGSGGGGSGGGGSEIVMTQ | |
| | | SPATLSLSPGERATLSCRASQDISKYLNWYQQKPGQA | |
| | | PRLLIYHTSRLHSGIPARFSGSGSGTDYTLTISSLQPED | |
| | | FAVYFCQQGNTLPYTFGQGTKLEIK | |
| CD19-scFv9 | scFv | EIVMTQSPATLSLSPGERATLSCRASQDISKYLNWYQQ | 104 |
| | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSGGGGSQVQLQESGPGLVKPSETLSLTCT | |
| | | VSGVSLPDYGVSWIRQPPGKGLEWIGVIWGSETTYYN | |
| | | SSLKSRVTISKDNSKNQVSLKLSSVTAADTAVYYCAKH | |
| | | YYYGGSYAMDYWGQGTLVTVSS | |
| CD19- | scFv | QVQLQESGPGLVKPSETLSLTCTVSGVSLPDYGVSWI | 105 |
| scFv10 | | RQPPGKGLEWIGVIWGSETTYYNSSLKSRVTISKDNSK | |
| | | NQVSLKLSSVTAADTAVYYCAKHYYYGGSYAMDYWG | |
| | | QGTLVTVSSGGGGSGGGGSGGGGSGGGGSEIVMTQ | |
| | | SPATLSLSPGERATLSCRASQDISKYLNWYQQKPGQA | |
| | | PRLLIYHTSRLHSGIPARFSGSGSGTDYTLTISSLQPED | |
| | | FAVYFCQQGNTLPYTFGQGTKLEIK | |
| CD19- | scFv | EIVMTQSPATLSLSPGERATLSCRASQDISKYLNWYQQ | 106 |
| scFv11 | | KPGQAPRLLIYHTSRLHSGIPARFSGSGSGTDYTLTISS | |
| | | LQPEDFAVYFCQQGNTLPYTFGQGTKLEIKGGGGSGG | |
| | | GGSGGGGSQVQLQESGPGLVKPSETLSLTCTVSGVS | |
| | | LPDYGVSWIRQPPGKGLEWIGVIWGSETTYYNSSLKS | |
| | | RVTISKDNSKNQVSLKLSSVTAADTAVYYCAKHYYYGG | |
| | | SYAMDYWGQGTLVTVSS | |
| CD19- | scFv | QVQLQESGPGLVKPSETLSLTCTVSGVSLPDYGVSWI | 107 |
| scFv12 | | RQPPGKGLEWIGVIWGSETTYYNSSLKSRVTISKDNSK | |
| | | NQVSLKLSSVTAADTAVYYCAKHYYYGGSYAMDYWG | |
| | | QGTLVTVSSGGGGSGGGGSGGGGSEIVMTQSPATLS | |
| | | LSPGERATLSCRASQDISKYLNWYQQKPGQAPRLLIYH | |
| | | TSRLHSGIPARFSGSGSGTDYTLTISSLQPEDFAVYFC | |
| | | QQGNTLPYTFGQGTKLEIK | |

In certain aspects, a CD19 binding molecule comprises heavy chain CDRs having the amino acid sequences of CD19-H1, CD19-1H2A, and CD19-1H3 as set forth in Table 10 and light chain CDRs having the amino acid sequences of CD19-L1, CD19-1L2, and CD19-1L3 as set forth in Table 1C. In a specific embodiment, a CD19 binding molecule comprises a heavy chain variable region having the amino acid sequences of VHA as set forth in Table 1C and a light chain variable region having the amino acid sequences of VLA as set forth in Table 1C.

In other aspects, a CD19 binding molecule comprises heavy chain CDRs having the amino acid sequences of CD19-H1, CD19-H2B, and CD19-H3 as set forth in Table 1C and light chain CDRs having the amino acid sequences of CD19-L1, CD19-L2, and CD19-L3 as set forth in Table 1C. In a specific embodiment, a CD19 binding molecule comprises a heavy chain variable region having the amino acid sequences of VHB as set forth in Table 1C and a light chain variable region having the amino acid sequences of VLB as set forth in Table 1C.

In further aspects, a CD19 binding molecule comprises heavy chain CDRs having the amino acid sequences of CD19-H1, CD19-H2C, and CD19-H3 as set forth in Table 1C and light chain CDRs having the amino acid sequences of CD19-L1, CD19-L2, and CD19-L3 as set forth in Table 1C. In a specific embodiment, a CD19 binding molecule comprises a heavy chain variable region having the amino acid sequences of VHC as set forth in Table 1C and a light chain variable region having the amino acid sequences of VLB as set forth in Table 1C.

In further aspects, a CD19 binding molecule comprises heavy chain CDRs having the amino acid sequences of CD19-H1, CD19-H2D, and CD19-H3 as set forth in Table 1C and light chain CDRs having the amino acid sequences of CD19-L1, CD19-L2, and CD19-L3 as set forth in Table 1C. In a specific embodiment, a CD19 binding molecule comprises a heavy chain variable region having the amino acid sequences of VHD as set forth in Table 1C and a light chain variable region having the amino acid sequences of VLB as set forth in Table 1C.

In yet further aspects, a CD19 binding molecule is in the form of an scFV. Exemplary anti-CD19 scFvs comprise the amino acid sequence of any one of CD19-scFv1 through CD19-scFv12 as set forth in Table 1C.

Other CD19 binding molecules include amino acids that have been mutated, yet have at least 80, 85, 90, 95, 96, 97, 98, or 99 percent identity in the CDR regions with the CDR sequences described in Table 1. In some embodiments, such CD19 binding molecules include mutant amino acid sequences where no more than 1, 2, 3, 4 or 5 amino acids have been mutated in the CDR regions when compared with the CDR sequences described in Table 1.

Other CD19 binding molecules include VH and/or VL domains comprising amino acid sequences having at least 80, 85, 90, 95, 96, 97, 98, or 99 percent identity to the VH and/or VL sequences described in Table 1. In some embodiments, CD19 binding molecules include VH and/or VL domains where no more than 1, 2, 3, 4 or 5 amino acids have been mutated when compared with the VH and/or VL domains depicted in the sequences described in Table 1, while retaining substantially the same therapeutic activity.

Additional CD19 binding molecules can be generated through the techniques of gene-shuffling, motif-shuffling, exon-shuffling, and/or codon-shuffling (collectively referred to as “DNA shuffling”). DNA shuffling can be employed to alter the activities of molecules of the disclosure or fragments thereof (e.g., molecules or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson et al., 1999, J. Mol. Biol. 287:265-76; and Lorenzo and Blasco, 1998, Biotechniques 24(2):308-313. The CD19 binding molecules described herein or fragments thereof can be altered by being subjected to random mutagenesis by error-prone PCR, random nucleotide insertion or other methods prior to recombination. A polynucleotide encoding a fragment of a CD19 binding molecule described herein can be recombined with one or more components, motifs, sections, parts, domains, fragments, etc. of one or more heterologous molecules.

Moreover, CD19 binding molecules can be fused to marker sequences, such as a peptide to facilitate purification. In some embodiments, the marker amino acid sequence is a hexa-histidine peptide (SEQ ID NO: 1253), such as the tag provided in a pQE vector (QIAGEN, Inc., 9259 Eton Avenue, Chatsworth, CA, 91311), among others, many of which are commercially available. As described in Gentz et al., 1989, Proc. Natl. Acad. Sci. USA 86:821-824, for instance, hexa-histidine (SEQ ID NO: 1253) provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984 Cell 37:767), and the “flag” tag.

Various other CD19 binding molecules, some of which are monospecific and some of which are multispecific, are known in the art and can also be used in the methods and combinations of the disclosure. See, for example, WO 2014/031687; WO 2012/079000; WO 2014/153270; U.S. Pat. No. 7,741,465; Naddafi et al., 2015, Int J Mol Cell Med. 4(3): 143-151; and Hammer, 2012, MAbs. 4(5): 571-577, the contents of which are incorporated herein by reference. In specific embodiments, the CD19 binding molecule is blinatumomab (Amgen), coltuximab ravtansine (Immunogen), MOR208 (also called XmAb-5574; Morphosys), MEDI-551 (Medlmmune), denintuzumab mafodotin (also called SGN-CD19A; Seattle Genetics), DI-B4 (Merck Serono), taplitumomabpaptox (National Cancer Institute), XmAb 5871 (Xencor), MDX-1342 (Bristol-Myers Squibb), AFM11 (Affimed), MDX-1342 (BMS), loncastuximab tesirine (ADC Therapeutics) or GBR401 (Glenmark).

### 7.2.1. Antigen Binding Modules of Multispecific Binding Molecules

In some aspects, one or more of the molecules used in the methods and combinations of the disclosure are multispecific binding molecules. For example, a CD19 binding molecule can in some embodiments be a multispecific binding molecule (MBM), e.g., a bispecific binding molecule (BBM) or trispecific binding molecule (TBM). Typically, one or more ABMs of the MBMs comprise immunoglobulin-based antigen-binding domains, for example the sequences of antibody fragments or derivatives. These antibody fragments and derivatives typically include the CDRs of an antibody and can include larger fragments and derivatives thereof, e.g., Fabs, scFabs, Fvs, and scFvs.

Immunoglobulin-based ABMs can comprise modifications to framework residues within a VH and/or a VL, e.g. to improve the properties of a MBM containing the ABM. For example, framework modifications can be made to decrease immunogenicity of a MBM. One approach for making such framework modifications is to “back-mutate” one or more framework residues of the ABM to a corresponding germline sequence. Such residues can be identified by comparing framework sequences to germline sequences from which the ABM is derived. To “match” framework region sequences to desired germline configuration, residues can be “back-mutated” to a corresponding germline sequence by, for example, site-directed mutagenesis. MBMs having such “back-mutated” ABMs are intended to be encompassed by the disclosure.

Another type of framework modification involves mutating one or more residues within a framework region, or even within one or more CDR regions, to remove T-cell epitopes to thereby reduce potential immunogenicity of a MBM. This approach is also referred to as “deimmunization” and is described in further detail in U.S. Patent Publication 20030153043 by Carr et al.

ABMs can also be modified to have altered glycosylation, which can be useful, for example, to increase the affinity of a MBM for one or more of its antigens. Such carbohydrate modifications can be accomplished by, for example, altering one or more sites of glycosylation within an ABM sequence. For example, one or more amino acid substitutions can be made that result in elimination of one or more variable region framework glycosylation sites to thereby eliminate glycosylation at that site. Such aglycosylation can increase the affinity of the MBM for an antigen. Such an approach is described in, e.g., U.S. Pat. Nos. 5,714,350 and 6,350,861 by Co et al.

### 7.2.1.1. Immunoglobulin Based ABMs

### 7.2.1.1.1. Fabs

In certain aspects, an ABM is a Fab domain.

For the MBMs of the disclosure, it is advantageous to use Fab heterodimerization strategies to permit the correct association of Fab domains belonging to the same ABM and minimize aberrant pairing of Fab domains belonging to different ABMs. For example, the Fab heterodimerization strategies shown in Table 2 below can be used:

TABLE 2
Fab Heterodimerization Strategies

| Name | STRATEGY | VH | CH1 | VL | CL | REFERENCE |
|---|---|---|---|---|---|---|
| F1 | CrossMabCH1-CL | WT | CL | WT | CH1 | Schaefer et al., 2011, |
| | | | domain | | domain | Cancer Cell 2011; |
| | | | | | | 20: 472-86; |
| | | | | | | PMID: 22014573. |
| F2 | orthogonal Fab | 39K, | H172A, | 1R, 38D, | L135Y, | Lewis et al., 2014, Nat |
| | VHVRD1CH1CRD2 - | 62E | F174G | (36F) | S176W | Biotechnol 32: 191-8 |
| | VLVRD1CλCRD2 | | | | | |
| F3 | orthogonal Fab | 39Y | WT | 38R | WT | Lewis et al., 2014, Nat |
| | VHVRD2CH1wt - | | | | | Biotechnol 32: 191-8 |
| | VLVRD2Cλwt | | | | | |
| F4 | TCR CαCβ | 39K | TCR Cα | 38D | TCR Cβ | Wu et al., 2015, MAbs |
| | | | | | | 7: 364-76 |
| F5 | CR3 | WT | T192E | WT | N137K, | Golay at al., 2016, J |
| | | | | | S114A | Immunol 196: 3199-211. |
| F6 | MUT4 | WT | L143Q, | WT | V133T, | Golay at al., 2016, J |
| | | | S188V | | S176V | Immunol 196: 3199-211. |
| F7 | DuetMab | WT | F126C | WT | S121C | Mazor et al., 2015, |
| | | | | | | MAbs 7: 377-89; Mazor |
| | | | | | | et al., 2015, MAbs |
| | | | | | | 7: 461-669. |

Accordingly, in certain embodiments, correct association between the two polypeptides of a Fab is promoted by exchanging the VL and VH domains of the Fab for each other or exchanging the CH1 and CL domains for each other, e.g., as described in WO 2009/080251.

Correct Fab pairing can also be promoted by introducing one or more amino acid modifications in the CH1 domain and one or more amino acid modifications in the CL domain of the Fab and/or one or more amino acid modifications in the VH domain and one or more amino acid modifications in the VL domain. The amino acids that are modified are typically part of the VH:VL and CH1:CL interface such that the Fab components preferentially pair with each other rather than with components of other Fabs.

In one embodiment, the one or amino acid modifications are limited to the conserved framework residues of the variable (VH, VL) and constant (CH1, CL) domains as indicated by the Kabat numbering of residues. Almagro, 2008, Frontiers In Bioscience 13:1619-1633 provides a definition of the framework residues on the basis of Kabat, Chothia, and IMGT numbering schemes.

In one embodiment, the modifications introduced in the VH and CH1 and/or VL and CL domains are complementary to each other. Complementarity at the heavy and light chain interface can be achieved on the basis of steric and hydrophobic contacts, electrostatic/charge interactions or a combination of the variety of interactions. The complementarity between protein surfaces is broadly described in the literature in terms of lock and key fit, knob into hole, protrusion and cavity, donor and acceptor etc., all implying the nature of structural and chemical match between the two interacting surfaces.

In one embodiment, the one or more introduced modifications introduce a new hydrogen bond across the interface of the Fab components. In one embodiment, the one or more introduced modifications introduce a new salt bridge across the interface of the Fab components. Exemplary substitutions are described in WO 2014/150973 and WO 2014/082179.

In some embodiments, the Fab domain comprises a 192E substitution in the CH1 domain and 114A and 137K substitutions in the CL domain, which introduces a salt-bridge between the CH1 and CL domains (see, Golay et al., 2016, J Immunol 196:3199-211).

In some embodiments, the Fab domain comprises a 143Q and 188V substitutions in the CH1 domain and 113T and 176V substitutions in the CL domain, which serves to swap hydrophobic and polar regions of contact between the CH1 and CL domain (see, Golay et al., 2016, J Immunol 196:3199-211).

In some embodiments, the Fab domain can comprise modifications in some or all of the VH, CH1, VL, CL domains to introduce orthogonal Fab interfaces which promote correct assembly of Fab domains (Lewis et al., 2014 Nature Biotechnology 32:191-198). In an embodiment, 39K, 62E modifications are introduced in the VH domain, H172A, F174G modifications are introduced in the CH1 domain, 1R, 38D, (36F) modifications are introduced in the VL domain, and L135Y, S176W modifications are introduced in the CL domain. In another embodiment, a 39Y modification is introduced in the VH domain and a 38R modification is introduced in the VL domain.

Fab domains can also be modified to replace the native CH1:CL disulfide bond with an engineered disulfide bond, thereby increasing the efficiency of Fab component pairing. For example, an engineered disulfide bond can be introduced by introducing a 126C in the CH1 domain and a 121C in the CL domain (see, Mazor et al., 2015, MAbs 7:377-89).

Fab domains can also be modified by replacing the CH1 domain and CL domain with alternative domains that promote correct assembly. For example, Wu et al., 2015, MAbs 7:364-76, describes substituting the CH1 domain with the constant domain of the a T cell receptor and substituting the CL domain with the p domain of the T cell receptor, and pairing these domain replacements with an additional charge-charge interaction between the VL and VH domains by introducing a 38D modification in the VL domain and a 39K modification in the VH domain.

ABMs can comprise a single chain Fab fragment, which is a polypeptide consisting of an antibody heavy chain variable domain (VH), an antibody constant domain 1 (CH1), an antibody light chain variable domain (VL), an antibody light chain constant domain (CL) and a linker. In some embodiments, the antibody domains and the linker have one of the following orders in N-terminal to C-terminal direction: a) VH—CH1-linker-VL-CL, b) VL-CL-linker-VH—CH1, c) VH-CL-linker-VL-CH1 or d) VL-CH1-linker-VH-CL. The linker can be a polypeptide of at least 30 amino acids, e.g., between 32 and 50 amino acids. The single chain Fab domains are stabilized via the natural disulfide bond between the CL domain and the CH1 domain.

In an embodiment, the antibody domains and the linker in the single chain Fab fragment have one of the following orders in N-terminal to C-terminal direction: a) VH—CH1-linker-VL-CL, or b) VL-CL-linker-VH—CH1. In some cases, VL-CL-linker-VH—CH1 is used.

In another embodiment, the antibody domains and the linker in the single chain Fab fragment have one of the following orders in N-terminal to C-terminal direction: a) VH-CL-linker-VL-CH1 or b) VL-CH1-linker-VH-CL.

Optionally in the single chain Fab fragment, additionally to the natural disulfide bond between the CL-domain and the CH1 domain, also the antibody heavy chain variable domain (VH) and the antibody light chain variable domain (VL)ABM are disulfide stabilized by introduction of a disulfide bond between the following positions: i) heavy chain variable domain position 44 to light chain variable domain position 100, ii) heavy chain variable domain position 105 to light chain variable domain position 43, or iii) heavy chain variable domain position 101 to light chain variable domain position 100 (numbering according to EU index of Kabat).

In one embodiment, the optional disulfide bond between the variable domains of the single chain Fab fragments is between heavy chain variable domain position 44 and light chain variable domain position 100. In one embodiment, the optional disulfide bond between the variable domains of the single chain Fab fragments is between heavy chain variable domain position 105 and light chain variable domain position 43 (numbering according to EU index of Kabat).

7.2.1.1.2. scFvs

In certain aspects, an ABM is a single chain Fv or “scFv”. Examples of linkers suitable for connecting the VH and VL chains of an scFV are the ABM linkers identified in Section 7.2.2.3, for example any of the linkers designated L1 through L54.

To create an scFv-encoding nucleic acid, the VH and VL-encoding DNA fragments are operably linked to another fragment encoding a linker, e.g., encoding any of the ABM linkers described in Section 7.2.2.3 (such as the amino acid sequence (Gly4˜Ser)3 (SEQ ID NO: 1174)

### 7.2.1.1.3. Other Immunoglobulin-Based ABMs

MBMs can also comprise ABMs having an immunoglobulin format which is other than Fab or scFv, for example Fv, dsFv, (Fab′)2, a single domain antibody (SDAB), a VH or VL domain, or a camelid VHH domain (also called a nanobody).

An ABM can be a single domain antibody composed of a single VH or VL domain which exhibits sufficient affinity to the target. In an embodiment, the single domain antibody is a camelid VHH domain (see, e.g., Riechmann, 1999, Journal of Immunological Methods 231:25-38; WO 94/04678).

### 7.2.1.2. Non-Immunoglobulin Based ABM

In certain embodiments, MBMs comprise one or more of the ABMs derived from non-antibody scaffold proteins (including, but not limited to, designed ankyrin repeat proteins (DARPins), Avimers (short for avidity multimers), Anticalin/Lipocalins, Centyrins, Kunitz domains, Adnexins, Affilins, Affitins (also known as Nonfitins), Knottins, Pronectins, Versabodies, Duocalins, and Fynomers), ligands, receptors, cytokines or chemokines.

Non-immunoglobulin scaffolds that can be used in the MBMs include those listed in Tables 3 and 4 of Mintz and Crea, 2013, Bioprocess International 11(2):40-48; in FIG. 1, Table 1 and FIG. I of Vazquez-Lombardi et al., 2015, Drug Discovery Today 20(10):1271-83; in Table 1 and Box 2 of Skrlec et al., 2015, Trends in Biotechnology 33(7):408-18. The contents of Tables 3 and 4 of Mintz and Crea, 2013, Bioprocess International 11(2):40-48; in FIG. 1, Table 1 and FIG. I of Vazquez-Lombardi et al., 2015, Drug Discovery Today 20(10):1271-83; in Table 1 and Box 2 of Skrlec et al., 2015, Trends in Biotechnology 33(7):408-18 (collectively, “Scaffold Disclosures”). In a particular embodiment, the Scaffold Disclosures are incorporated by reference for what they disclose relating to Adnexins. In another embodiment, the Scaffold Disclosures are incorporated by reference for what they disclose relating to Avimers. In another embodiment, the Scaffold Disclosures are incorporated by reference for what they disclose relating to Affibodies. In yet another embodiment, the Scaffold Disclosures are incorporated by reference for what they disclose relating to Anticalins. In yet another embodiment, the Scaffold Disclosures are incorporated by reference for what they disclose relating to DARPins. In yet another embodiment, the Scaffold Disclosures are incorporated by reference for what they disclose relating to Kunitz domains. In yet another embodiment, the Scaffold Disclosures are incorporated by reference for what they disclose relating to Knottins. In yet another embodiment, the Scaffold Disclosures are incorporated by reference for what they disclose relating to Pronectins. In yet another embodiment, the Scaffold Disclosures are incorporated by reference for what they disclose relating to Nanofitins. In yet another embodiment, the Scaffold Disclosures are incorporated by reference for what they disclose relating to Affilins. In yet another embodiment, the Scaffold Disclosures are incorporated by reference for what they disclose relating to Adnectins. In yet another embodiment, the Scaffold Disclosures are incorporated by reference for what they disclose relating to ABMs. In yet another embodiment, the Scaffold Disclosures are incorporated by reference for what they disclose relating to Adhirons. In yet another embodiment, the Scaffold Disclosures are incorporated by reference for what they disclose relating to Affimers. In yet another embodiment, the Scaffold Disclosures are incorporated by reference for what they disclose relating to Alphabodies. In yet another embodiment, the Scaffold Disclosures are incorporated by reference for what they disclose relating to Armadillo Repeat Proteins. In yet another embodiment, the Scaffold Disclosures are incorporated by reference for what they disclose relating to Atrimers/Tetranectins. In yet another embodiment, the Scaffold Disclosures are incorporated by reference for what they disclose relating to Obodies/OB-folds. In yet another embodiment, the Scaffold Disclosures are incorporated by reference for what they disclose relating to Centyrins. In yet another embodiment, the Scaffold Disclosures are incorporated by reference for what they disclose relating to Repebodies. In yet another embodiment, the Scaffold Disclosures are incorporated by reference for what they disclose relating to Anticalins. In yet another embodiment, the Scaffold Disclosures are incorporated by reference for what they disclose relating to Atrimers. In yet another embodiment, the Scaffold Disclosures are incorporated by reference for what they disclose relating to bicyclic peptides. In yet another embodiment, the Scaffold Disclosures are incorporated by reference for what they disclose relating to cys-knots. In yet another embodiment, the Scaffold Disclosures are incorporated by reference for what they disclose relating to Fn3 scaffolds (including Adnectins, Centryrins, Pronectins, and Tn3).

In an embodiment, an ABM can be a designed ankyrin repeat protein (“DARPin”). DARPins are antibody mimetic proteins that typically exhibit highly specific and high-affinity target protein binding. They are typically genetically engineered and derived from natural ankyrin proteins and consist of at least three, usually four or five repeat motifs of these proteins. Their molecular mass is about 14 or 18 kDa (kilodaltons) for four- or five-repeat DARPins, respectively. Examples of DARPins can be found, for example in U.S. Pat. No. 7,417,130. Multispecific binding molecules comprising DARPin binding modules and immunoglobulin-based binding modules are disclosed in, for example, U.S. Publication No. 2015/0030596 A1.

In another embodiment, an ABM can be an Affibody. An Affibody is well known and refers to affinity proteins based on a 58 amino acid residue protein domain, derived from one of the IgG binding domain of staphylococcal protein A.

In another embodiment, an ABM can be an Anticalin. Anticalins are well known and refer to another antibody mimetic technology, where the binding specificity is derived from Lipocalins. Anticalins can also be formatted as dual targeting protein, called Duocalins.

In another embodiment, an ABM can be a Versabody. Versabodies are well known and refer to another antibody mimetic technology. They are small proteins of 3-5 kDa with >15% cysteines, which form a high disulfide density scaffold, replacing the hydrophobic core of typical proteins.

Other non-immunoglobulin ABMs include “A” domain oligomers (also known as Avimers) (see for example, U.S. Patent Application Publication Nos. 2005/0164301, 2005/0048512, and 2004/017576), Fn3 based protein scaffolds (see for example, U.S. Patent Application Publication 2003/0170753), VASP polypeptides, Avian pancreatic polypeptide (aPP), Tetranectin (based on CTLD3), Affililin (based on yB-crystallin/ubiquitin), Knottins, SH3 domains, PDZ domains, Tendamistat, Neocarzinostatin, Protein A domains, Lipocalins, Transferrin, or Kunitz domains. In one aspect, ABMs useful in the construction of the MBMs comprise fibronectin-based scaffolds as exemplified in WO 2011/130324.

Moreover, in certain aspects, an ABM comprises a ligand binding domain of a receptor or a receptor binding domain of a ligand.

### 7.2.2. Connectors

It is contemplated that the CD19 binding molecules can in some instances include pairs of ABMs or ABM chains (e.g., the VH—CH1 or VL-CL component of a Fab) connected directly to one another, e.g., as a fusion protein without a linker. For example, the CD19 binding molecules can comprise connector moieties linking individual ABMs or ABM chains. The use of connector moieties can improve target binding, for example by increasing flexibility of the ABMs within a CD19 binding molecule and thus reducing steric hindrance. The ABMs or ABM chains can be connected to one another through, for example, Fc domains (each Fc domain representing a pair of associated Fc regions) and/or ABM linkers. The use of Fc domains will typically require the use of hinge regions as connectors of the ABMs or ABM chains for optimal antigen binding. Thus, the term “connector” encompasses, but is not limited to, Fc regions, Fc domains, and hinge regions.

Connectors can be selected or modified to, for example, increase or decrease the biological half-life of a CD19 binding molecule. For example, to decrease biological half-life, one or more amino acid mutations can be introduced into a CH2-CH3 domain interface region of an Fc-hinge fragment such that a CD19 binding molecule comprising the fragment has impaired Staphylococcyl Protein A (SpA) binding relative to native Fc-hinge domain SpA binding. This approach is described in further detail in U.S. Pat. No. 6,165,745 by Ward et al. Alternatively, a CD19 binding molecule can be modified to increase its biological half-life. For example, one or more of the following mutations can be introduced: T252L, T254S, T256F, as described in U.S. Pat. No. 6,277,375 to Ward. Alternatively, to increase the biological half-life, a CD19 binding molecule can be altered within a CH1 or CL region to contain a salvage receptor binding epitope taken from two loops of a CH2 domain of an Fc region of an IgG, as described in U.S. Pat. Nos. 5,869,046 and 6,121,022 by Presta et al.

Examples of Fc domains (formed by the pairing of two Fc regions), hinge regions and ABM linkers are described in Sections 7.2.2.1, 7.2.2.2, and 7.2.2.3, respectively.

### 7.2.2.1. Fc Domains

The CD19 binding molecules can include an Fc domain derived from any suitable species. In one embodiment, the Fc domain is derived from a human Fc domain.

The Fc domain can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgG1, IgG2, IgG3 or IgG4. In one embodiment, the Fc domain is derived from IgG1. In one embodiment, the Fc domain is derived from IgG4.

The Fc domain comprises two polypeptide chains, each referred to as a heavy chain Fc region. The two heavy chain Fc regions dimerize to create the Fc domain. The two Fc regions within the Fc domain can be the same or different from one another. In a native antibody the Fc regions are typically identical, but for the purpose of producing multispecific binding molecules of the disclosure, the Fc regions might advantageously be different to allow for heterodimerization, as described in Section 7.2.2.1.5 below.

Typically each heavy chain Fc region comprises or consists of two or three heavy chain constant domains.

In native antibodies, the heavy chain Fc region of IgA, IgD and IgG is composed of two heavy chain constant domains (CH2 and CH3) and that of IgE and IgM is composed of three heavy chain constant domains (CH2, CH3 and CH4). These dimerize to create an Fc domain.

In the present disclosure, the heavy chain Fc region can comprise heavy chain constant domains from one or more different classes of antibody, for example one, two or three different classes.

In one embodiment, the heavy chain Fc region comprises CH2 and CH3 domains derived from IgG1. An exemplary sequence of a heavy chain Fc region derived from human IgG1 is given in SEQ ID NO:251:

(SEQ ID NO: 251)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSR
WQQGNVFSCSVMHEALHNHYTQKSLSLSP.

In some embodiments, a CD19 binding molecule comprises a Fc region whose amino acid sequence comprises the amino acid sequence of SEQ ID NO:251 modified with one or more of the substitutions described in Section 7.2.2.1 and its subparts.

In one embodiment, the heavy chain Fc region comprises CH2 and CH3 domains derived from IgG2.

In one embodiment, the heavy chain Fc region comprises CH2 and CH3 domains derived from IgG3.

In one embodiment, the heavy chain Fc region comprises CH2 and CH3 domains derived from IgG4.

In one embodiment, the heavy chain Fc region comprises a CH4 domain from IgM. The IgM CH4 domain is typically located at the C-terminus of the CH3 domain.

In one embodiment, the heavy chain Fc region comprises CH2 and CH3 domains derived from IgG and a CH4 domain derived from IgM.

It will be appreciated that the heavy chain constant domains for use in producing a heavy chain Fc region for the CD19 binding molecules of the present disclosure can include variants of the naturally occurring constant domains described above. Such variants can comprise one or more amino acid variations compared to wild type constant domains. In one example the heavy chain Fc region of the present disclosure comprises at least one constant domain that varies in sequence from the wild type constant domain. It will be appreciated that the variant constant domains can be longer or shorter than the wild type constant domain. For example, the variant constant domains are at least 60% identical or similar to a wild type constant domain. In another example the variant constant domains are at least 70% identical or similar. In another example the variant constant domains are at least 75% identical or similar. In another example the variant constant domains are at least 80% identical or similar. In another example the variant constant domains are at least 85% identical or similar. In another example the variant constant domains are at least 90% identical or similar. In another example the variant constant domains are at least 95% identical or similar. In another example the variant constant domains are at least 99% identical or similar. Exemplary Fc variants are described in Sections 7.2.2.1.1 through 7.2.2.1.6, infra.

IgM and IgA occur naturally in humans as covalent multimers of the common H2L2 antibody unit. IgM occurs as a pentamer when it has incorporated a J-chain, or as a hexamer when it lacks a J-chain. IgA occurs as monomer and dimer forms. The heavy chains of IgM and IgA possess an 18 amino acid extension to the C-terminal constant domain, known as a tailpiece. The tailpiece includes a cysteine residue that forms a disulfide bond between heavy chains in the polymer, and is believed to have an important role in polymerization. The tailpiece also contains a glycosylation site. In certain embodiments, the CD19 binding molecules of the present disclosure do not comprise a tailpiece.

The Fc domains that are incorporated into the CD19 binding molecules can comprise one or more modifications that alter one or more functional properties of the proteins, such as serum half-life, complement fixation, Fc receptor binding, and/or antigen-dependent cellular cytotoxicity. Furthermore, a CD19 binding molecule can be chemically modified (e.g., one or more chemical moieties can be attached to the CD19 binding molecule) or be modified to alter its glycosylation, again to alter one or more functional properties of the CD19 binding molecule.

Effector function of an antibody molecule includes complement-mediated effector function, which is mediated by, for example, binding of the C1 component of the complement to the antibody. Activation of complement is important in the opsonization and direct lysis of pathogens. In addition, it stimulates the inflammatory response by recruiting and activating phagocytes to the site of complement activation. Effector function includes Fc receptor (FcR)-mediated effector function, which can be triggered upon binding of the constant domains of an antibody to an Fc receptor (FcR). Antigen-antibody complex-mediated crosslinking of Fc receptors on effector cell surfaces triggers a number of important and diverse biological responses including engulfment and destruction of antibody-coated particles, clearance of immune complexes, Iysis of antibody-coated target cells by killer cells (called antibody-dependent cell-mediated cytotoxicity, or ADCC), release of inflammatory mediators, placental transfer and control of immunoglobulin production.

Fc regions can be altered by replacing at least one amino acid residue with a different amino acid residue to alter the effector functions. For example, one or more amino acids can be replaced with a different amino acid residue such that the Fc region has an altered affinity for an effector ligand. The effector ligand to which affinity is altered can be, for example, an Fc receptor or the C1 component of complement. This approach is described in, e.g., U.S. Pat. Nos. 5,624,821 and 5,648,260, both by Winter et al. Modified Fc regions can also alter C1q binding and/or reduce or abolish complement dependent cytotoxicity (CDC). This approach is described in, e.g., U.S. Pat. No. 6,194,551 by Idusogie et al. Modified Fc regions can also alter the ability of an Fc region to fix complement. This approach is described in, e.g., the PCT Publication WO 94/29351 by Bodmer et al. Allotypic amino acid residues include, but are not limited to, constant region of a heavy chain of the IgG1, IgG2, and IgG3 subclasses as well as constant region of a light chain of the kappa isotype as described by Jefferis et al., 2009, MAbs, 1:332-338.

Fc regions can also be modified to “silence” the effector function, for example, to reduce or eliminate the ability of a CD19 binding molecule to mediate antibody dependent cellular cytotoxicity (ADCC) and/or antibody dependent cellular phagocytosis (ADCP). This can be achieved, for example, by introducing a mutation in an Fc region. Such mutations have been described in the art: LALA and N297A (Strohl, 2009, Curr. Opin. Biotechnol. 20(6):685-691); and D265A (Baudino et al., 2008, J. Immunol. 181: 6664-69; Strohl, supra). Examples of silent Fc IgG1 antibodies comprise the so-called LALA mutant comprising L234A and L235A mutation in the IgG1 Fc amino acid sequence. Another example of a silent IgG1 antibody comprises the D265A mutation. Another silent IgG1 antibody comprises the so-called DAPA mutant comprising D265A and P329A mutations in the IgG1 Fc amino acid sequence. Another silent IgG1 antibody comprises the N297A mutation, which results in aglycosylated/non-glycosylated antibodies.

Fc regions can be modified to increase the ability of a CD19 binding molecule containing the Fc region to mediate antibody dependent cellular cytotoxicity (ADCC) and/or antibody dependent cellular phagocytosis (ADCP), for example, by modifying one or more amino acid residues to increase the affinity of the CD19 binding molecule for an activating Fcγ receptor, or to decrease the affinity of the CD19 binding molecule for an inhibitory Fcγ receptor. Human activating Fcγ receptors include FcγRIa, FcγRIIa, FcγRIIIa, and FcγRIIIb, and human inhibitory Fcγ receptor includes FcγRIIb. This approach is described in, e.g., the PCT Publication WO 00/42072 by Presta. Moreover, binding sites on human IgG1 for FcγRI, FcγRII, FcγRIII and FcRn have been mapped and variants with improved binding have been described (see Shields et al., J. Biol. Chem. 276:6591-6604, 2001). Optimization of Fc-mediated effector functions of monoclonal antibodies such as increased ADCC/ADCP function has been described (see Strohl, 2009, Current Opinion in Biotechnology 20:685-691). Mutations that can enhance ADCC/ADCP function include one or more mutations selected from G236A, S239D, F243L, P2471, D280H, K290S, R292P, S298A, S298D, S298V, Y300L, V3051, A330L, 1332E, E333A, K334A, A339D, A339Q, A339T, and P396L (all positions by EU numbering).

Fc regions can also be modified to increase the ability of a CD19 binding molecule to mediate ADCC and/or ADCP, for example, by modifying one or more amino acids to increase the affinity of the CD19 binding molecule for an activating receptor that would typically not recognize the parent CD19 binding molecule, such as FcαRI. This approach is described in, e.g., Borrok et al., 2015, mAbs. 7(4):743-751.

Accordingly, in certain aspects, the CD19 binding molecules can include Fc domains with altered effector function such as, but not limited to, binding to Fc-receptors such as FcRn or leukocyte receptors (for example, as described above or in Section 7.2.2.1.1), binding to complement (for example as described above or in Section 7.2.2.1.2), modified disulfide bond architecture (for example as described above or in Section 7.2.2.1.3), or altered glycosylation patterns (for example as described above or in Section 7.2.2.1.4). The Fc domains can also be altered to include modifications that improve manufacturability of asymmetric CD19 binding molecules, for example by allowing heterodimerization, which is the preferential pairing of non-identical Fc regions over identical Fc regions. Heterodimerization permits the production of CD19 binding molecules in which different ABMs are connected to one another by an Fc domain containing Fc regions that differ in sequence. Examples of heterodimerization strategies are exemplified in Section 7.2.2.1.5 (and subsections thereof).

It will be appreciated that any of the modifications described in Sections 7.2.2.1.1 through 7.2.2.1.5 can be combined in any suitable manner to achieve the desired functional properties and/or combined with other modifications to alter the properties of the CD19 binding molecules. In some embodiments, a CD19 binding molecule comprises a IgG1 Fc domain having a mutation at 1, 2, 3, 4, 5, 6, or more than 6 of positions 233, 234, 235, 236, 237, 239, 265, 266, 267, 268, 269, 297, 299, 322, 327, 328, 329, 330, 331 and 332 (EU numbering). For example, a CD19 binding molecule can comprise an IgG1 sequence of SEQ ID NO:251 with a mutation at 1, 2, 3, 4, 5, 6, or more than 6 of positions 233, 234, 235, 236, 237, 239, 265, 266, 267, 268, 269, 297, 299, 322, 327, 328, 329, 330, 331 and 332.

In some embodiments, a CD19 binding molecule comprises a first and second human IgG1 Fc region having amino acid substitutions selected from the following combinations of substitutions: substitutions L234A, L235A, and G237A (“LALAGA”); substitutions L234A, L235A, S267K, and P329A (“LALASKPA”); subsitutions D265A, P329A, and S267K (“DAPASK”); substitutions G237A, D265A, and P329A (“GADAPA”); substitutions G237A, D265A, P329A, and S267K (“GADAPASK”); substitutions L234A, L235A, and P329G (“LALAPG”), and substitutions L234A, L235A, and P329A (“LALAPA”), wherein the amino acid residues are numbered according to the EU numbering system. It should be understood that the terms “LALAGA”, “LALASKPA”, “DAPASK”, “GADAPA”, “GADAPASK”, “LALAPG”, and “LALAPA” represent shorthand terminology for the different combinations of subsitutions described in this paragraph rather than contiguous amino acid sequences.

In another embodiment, a CD19 binding molecule comprises a human IgG1 Fc region having amino acid substitutions selected from the combinations of substitutions L234A, L235A, S267K, P329A (“LALASKPA”), or substitutions G237A, D265A, P329A, S267K (“GADAPASK”), wherein the amino acid residues are numbered according to the EU numbering system.

In a further embodiment, a CD19 binding molecules comprises a Fc region selected from FCV1-FCV7. (See Table A below)

In yet a further embodiment, a CD19 binding molecules comprises a Fc region which is FCV4 or FCV7.

In some aspects, the CD19 binding molecule has reduced or undetectable binding affinity to a Fc gamma receptor or C1q compared to a polypeptide comprising the wild-type human IgG1 Fc region optionally measured by surface plasmon resonance using a Biacore T200 instrument, wherein the Fc gamma receptor is selected from the group consisting of Fc gamma RIA, Fc gamma RIIIa V158 variant and Fc gamma RIIIa F158 variant, and wherein the binding compared to wildtype is reduced by 50%, 80%, 90%, 95%, 98%, 99% or is undetectable.

In some aspects, the CD19 binding molecule has reduced or undetectable effector function compared to a polypeptide comprising the wild-type human IgG1 Fc region.

In some aspects, the CD19 binding molecule is capable of binding to an antigen without triggering detectable antibody-dependent cell-mediated cytotoxicity (ADCC), antibody-dependent cellular phagocytosis (ADCP), or complement dependent cytotoxicity (CDC). In some aspects, the effector function to be reduced or diminished is antibody-dependent cell-mediated cytotoxicity (ADCC) in the individual. In some aspects, the effector function to be reduced or diminished is antibody-dependent cellular phagocytosis (ADCP) in the individual. In some aspects, the effector function to be reduced or diminished is complement dependent cytotoxicity (CDC) in the individual. In some aspects, the first and second Fc regions of a Fc domain each comprise a nucleic acid sequence selected from a nucleic acid sequence listed in Table A below, or any sequence having at least about 90%, 91% 92%, 93%, 94%, 95%, 96%, 97%, 98%, 99%, or 100% identity thereto.

In an embodiment, a nucleic acid encoding a Fc region comprises the nucleic acid sequence of FCV-7 (see Table A below), or a sequence having at least about 90%, 91% 92%, 93%, 94%, 95%, 96%, 97%, 98%, 99%, or 100% identity thereto. In an embodiment, a nucleic acid encoding a Fc region comprises the nucleic acid sequence of FCV-4 (see Table A below), or a sequence having at least about 90%, 91% 92%, 93%, 94%, 95%, 96%, 97%, 98%, 99%, or 100% identity thereto. In some aspects, a Fc domain comprises first and second Fc regions each of which comprises an amino acid sequence selected from an amino acid sequence listed in Table A below, or any sequence having at least about 90%, 91% 92%, 93%, 94%, 95%, 96%, 97%, 98%, 99%, or 100% identity thereto.

In an embodiment, a Fc domain comprises first and second Fc regions comprising the amino acid sequence of FCV-7 (see Table A below), or a sequence having at least about 90%, 91% 92%, 93%, 94%, 95%, 96%, 97%, 98%, 99%, or 100% identity thereto. In an embodiment, a Fc domain comprises first and second Fc regions comprising the amino acid sequence of FCV-4 (see Table A below), or a sequence having at least about 90%, 91% 92%, 93%, 94%, 95%, 96%, 97%, 98%, 99%, or 100% identity thereto.

Additionally provided herein are vectors comprising the polynucleotides encoding CD19 binding molecules comprising a Fc region selected from FCV1-FCV7. (See Table A below)

Also provided herein are host cells comprising vectors or polynucleotides encoding and capable of expressing CD19 binding molecules comprising a Fc region selected from FCV1-FCV7. (See Table A below).

TABLE A
Sequences of Fc variants

| SEQ ID | | |
|---|---|---|
| No | Description | Sequence |
| 1254 | FCV-1: Human IgG1 | apeaaggpsvflfppkpkdtlmisrtpevtcvvvdvshedpev |
| | Fc variant | kfnwyvdgvevhnaktkpreeqynstyrvvsvltvlhqdwlng |
| | L234A/L235A/P329A | keykckvsnkalaapiektiskakgqprepqvytlppsrdelt |
| | (LALAPA) | knqvsltclvkgfypsdiavewesngqpennykttppvldsdg |
| | Amino acid sequence | sfflyskltvdksrwqqgnvfscsvmhealhnhytqkslslsp |
| | | gk |
| 1255 | FCV-1: Human IgG1 | gcccctgaagccgccggcggaccctccgtgttcctgttccccc |
| | Fc variant | caaagcccaaggacaccctgatgatcagccggacccccgaagt |
| | L234A/L235A/P329A | gacctgcgtggtggtggacgtgtcccacgaggaccctgaagtg |
| | (LALAPA) | aagttcaattggtacgtggacggcgtggaagtgcacaacgcca |
| | Nucleic acid | agaccaagcccagagaggaacagtacaacagcacctaccgggt |
| | sequence | ggtgtccgtgctgaccgtgctgcaccaggactggctgaacggc |
| | | aaagagtacaagtgcaaagtctccaacaaggccctggccgccc |
| | | ccatcgagaaaaccatcagcaaggccaagggccagccccgcga |
| | | gccccaggtgtacacactgccccccagccgggacgagctgacc |
| | | aagaaccaggtgtccctgacctgcctggtcaagggcttctacc |
| | | ccagcgatatcgccgtggaatgggagagcaacggccagcccga |
| | | gaacaactacaagaccaccccccctgtgctggacagcgacggc |
| | | tcattcttcctgtacagcaagctgaccgtggacaagtcccggt |
| | | ggcagcagggcaacgtgttcagctgcagcgtgatgcacgaggc |
| | | cctgcacaaccactacacccagaagtccctgagcctgagcccc |
| | | ggcaaa |
| 1256 | FCV-2: Human IgG1 | apeaagapsvflfppkpkdtlmisrtpevtcvvvdvshedpev |
| | Fc variant | kfnwyvdgvevhnaktkpreeqynstyrvvsvltvlhqdwlng |
| | L234A/L235A/G237A | keykckvsnkalpapiektiskakgqprepqvytlppsrdelt |
| | (LALAGA) | knqvsltclvkgfypsdiavewesngqpennykttppvldsdg |
| | Amino acid sequence | sfflyskltvdksrwqqgnvfscsvmhealhnhytqkslslsp |
| | | gk |
| 1257 | FCV-2: Human IgG1 | gcccctgaagccgccggcgccccctccgtgttcctgttccccc |
| | Fc variant | caaagcccaaggacaccctgatgatcagccggacccccgaagt |
| | L234A/L235A/G237A | gacctgcgtggtggtggacgtgtcccacgaggaccctgaagtg |
| | (LALAGA) | aagttcaattggtacgtggacggcgtggaagtgcacaacgcca |
| | Nucleic acid | agaccaagcccagagaggaacagtacaacagcacctaccgggt |
| | sequence | ggtgtccgtgctgaccgtgctgcaccaggactggctgaacggc |
| | | aaagagtacaagtgcaaagtctccaacaaggccctgcctgccc |
| | | ccatcgagaaaaccatcagcaaggccaagggccagccccgcga |
| | | gccccaggtgtacacactgccccccagccgggacgagctgacc |
| | | aagaaccaggtgtccctgacctgcctggtcaagggcttctacc |
| | | ccagcgatatcgccgtggaatgggagagcaacggccagcccga |
| | | gaacaactacaagaccaccccccctgtgctggacagcgacggc |
| | | tcattcttcctgtacagcaagctgaccgtggacaagtcccggt |
| | | ggcagcagggcaacgtgttcagctgcagcgtgatgcacgaggc |
| | | cctgcacaaccactacacccagaagtccctgagcctgagcccc |
| | | ggcaaa |
| 1258 | FCV-3: Human IgG1 | apeaaggpsvflfppkpkdtlmisrtpevtcvvvdvshedpev |
| | Fc variant | kfnwyvdgvevhnaktkpreeqynstyrvvsvltvlhqdwlng |
| | L234A/L235A/P329G | keykckvsnkalgapiektiskakgqprepqvytlppsrdelt |
| | (LALAPG) | knqvsltclvkgfypsdiavewesngqpennykttppvldsdg |
| | Amino acid sequence | sfflyskltvdksrwqqgnvfscsvmhealhnhytqkslslsp |
| | | gk |
| 1259 | FCV-3: Human IgG1 | gcccctgaagccgccggcggaccctccgtgttcctgttccccc |
| | Fc variant | caaagcccaaggacaccctgatgatcagccggacccccgaagt |
| | L234A/L235A/P329G | gacctgcgtggtggtggacgtgtcccacgaggaccctgaagtg |
| | (LALAPG) | aagttcaattggtacgtggacggcgtggaagtgcacaacgcca |
| | Nucleic acid | agaccaagcccagagaggaacagtacaacagcacctaccgggt |
| | sequence | ggtgtccgtgctgaccgtgctgcaccaggactggctgaacggc |
| | | aaagagtacaagtgcaaagtctccaacaaggccctgggcgccc |
| | | ccatcgagaaaaccatcagcaaggccaagggccagccccgcga |
| | | gccccaggtgtacacactgccccccagccgggacgagctgacc |
| | | aagaaccaggtgtccctgacctgcctggtcaagggcttctacc |
| | | ccagcgatatcgccgtggaatgggagagcaacggccagcccga |
| | | gaacaactacaagaccaccccccctgtgctggacagcgacggc |
| | | tcattcttcctgtacagcaagctgaccgtggacaagtcccggt |
| | | ggcagcagggcaacgtgttcagctgcagcgtgatgcacgaggc |
| | | cctgcacaaccactacacccagaagtccctgagcctgagcccc |
| | | ggcaaa |
| 1260 | FCV-4: Human IgG1 | apeaaggpsvflfppkpkdtlmisrtpevtcvvvdvkhedpev |
| | Fc variant | kfnwyvdgvevhnaktkpreeqynstyrvvsvltvlhqdwlng |
| | L234A/L235A/S267K/ | keykckvsnkalaapiektiskakgqprepqvytlppsrdelt |
| | P329A (LALASKPA) | knqvsltclvkgfypsdiavewesngqpennykttppvldsdg |
| | Amino acid sequence | sfflyskltvdksrwqqgnvfscsvmhealhnhytqkslslsp |
| | | gk |
| 1261 | FCV-4: Human IgG1 | gcccctgaagccgccggcggaccctccgtgttcctgttccccc |
| | Fc variant | caaagcccaaggacaccctgatgatcagccggacccccgaagt |
| | L234A/L235A/S267K/ | gacctgcgtggtggtggacgtgaagcacgaggaccctgaagtg |
| | P329A (LALASKPA) | aagttcaattggtacgtggacggcgtggaagtgcacaacgcca |
| | Nucleic acid | agaccaagcccagagaggaacagtacaacagcacctaccgggt |
| | sequence | ggtgtccgtgctgaccgtgctgcaccaggactggctgaacggc |
| | | aaagagtacaagtgcaaagtctccaacaaggccctggccgccc |
| | | ccatcgagaaaaccatcagcaaggccaagggccagccccgcga |
| | | gccccaggtgtacacactgccccccagccgggacgagctgacc |
| | | aagaaccaggtgtccctgacctgcctggtcaagggcttctacc |
| | | ccagcgatatcgccgtggaatgggagagcaacggccagcccga |
| | | gaacaactacaagaccaccccccctgtgctggacagcgacggc |
| | | tcattcttcctgtacagcaagctgaccgtggacaagtcccggt |
| | | ggcagcagggcaacgtgttcagctgcagcgtgatgcacgaggc |
| | | cctgcacaaccactacacccagaagtccctgagcctgagcccc |
| | | ggcaaa |
| 1262 | FCV-5: Human IgG1 | apellggpsvflfppkpkdtlmisrtpevtcvvvavkhedpev |
| | Fc variant | kfnwyvdgvevhnaktkpreeqynstyrvvsvltvlhqdwlng |
| | D265A/P329A/S267K | keykckvsnkalaapiektiskakgqprepqvytlppsrdelt |
| | (DAPASK) | knqvsltclvkgfypsdiavewesngqpennykttppvldsdg |
| | Amino acid sequence | sfflyskltvdksrwqqgnvfscsvmhealhnhytqkslslsp |
| | | gk |
| 1263 | FCV-5: Human IgG1 | gcccctgaactgctgggcggaccctccgtgttcctgttccccc |
| | Fc variant | caaagcccaaggacaccctgatgatcagccggacccccgaagt |
| | D265A/P329A/S267K | gacctgcgtggtggtggccgtgaagcacgaggaccctgaagtg |
| | (DAPASK) | aagttcaattggtacgtggacggcgtggaagtgcacaacgcca |
| | Nucleic acid | agaccaagcccagagaggaacagtacaacagcacctaccgggt |
| | sequence | ggtgtccgtgctgaccgtgctgcaccaggactggctgaacggc |
| | | aaagagtacaagtgcaaagtctccaacaaggccctggccgccc |
| | | ccatcgagaaaaccatcagcaaggccaagggccagccccgcga |
| | | gccccaggtgtacacactgccccccagccgggacgagctgacc |
| | | aagaaccaggtgtccctgacctgcctggtcaagggcttctacc |
| | | ccagcgatatcgccgtggaatgggagagcaacggccagcccga |
| | | gaacaactacaagaccaccccccctgtgctggacagcgacggc |
| | | tcattcttcctgtacagcaagctgaccgtggacaagtcccggt |
| | | ggcagcagggcaacgtgttcagctgcagcgtgatgcacgaggc |
| | | cctgcacaaccactacacccagaagtccctgagcctgagcccc |
| | | ggcaaa |
| 1264 | FCV-6: Human IgG1 | apellgapsvflfppkpkdtlmisrtpevtcvvvavshedpev |
| | Fc variant | kfnwyvdgvevhnaktkpreeqynstyrvvsvltvlhqdwlng |
| | G237A/D265A/P329A | keykckvsnkalaapiektiskakgqprepqvytlppsrdelt |
| | (GADAPA) | knqvsltclvkgfypsdiavewesngqpennykttppvldsdg |
| | Amino acid sequence | sfflyskltvdksrwqqgnvfscsvmhealhnhytqkslslsp |
| | | gk |
| 1265 | FCV-6: Human IgG1 | gcccctgaactgctgggcgccccctccgtgttcctgttccccc |
| | Fc variant | caaagcccaaggacaccctgatgatcagccggacccccgaagt |
| | G237A/D265A/P329A | gacctgcgtggtggtggccgtgtcccacgaggaccctgaagtg |
| | (GADAPA) | aagttcaattggtacgtggacggcgtggaagtgcacaacgcca |
| | Nucleic acid | agaccaagcccagagaggaacagtacaacagcacctaccgggt |
| | sequence | ggtgtccgtgctgaccgtgctgcaccaggactggctgaacggc |
| | | aaagagtacaagtgcaaagtctccaacaaggccctggccgccc |
| | | ccatcgagaaaaccatcagcaaggccaagggccagccccgcga |
| | | gccccaggtgtacacactgccccccagccgggacgagctgacc |
| | | aagaaccaggtgtccctgacctgcctggtcaagggcttctacc |
| | | ccagcgatatcgccgtggaatgggagagcaacggccagcccga |
| | | gaacaactacaagaccaccccccctgtgctggacagcgacggc |
| | | tcattcttcctgtacagcaagctgaccgtggacaagtcccggt |
| | | ggcagcagggcaacgtgttcagctgcagcgtgatgcacgaggc |
| | | cctgcacaaccactacacccagaagtccctgagcctgagcccc |
| | | ggcaaa |
| 1266 | FCV-7: Human IgG1 | apellgapsvflfppkpkdtlmisrtpevtcvvvavkhedpev |
| | Fc variant | kfnwyvdgvevhnaktkpreeqynstyrvvsvltvlhqdwlng |
| | G237A/D265A/P329A/ | keykckvsnkalaapiektiskakgqprepqvytlppsrdelt |
| | S267K (GADAPASK) | knqvsltclvkgfypsdiavewesngqpennykttppvldsdg |
| | Amino acid sequence | sfflyskltvdksrwqqgnvfscsvmhealhnhytqkslslsp |
| | | gk |
| 1267 | FCV-7: Human IgG1 | gcccctgaactgctgggcgccccctccgtgttcctgttccccc |
| | Fc variant | caaagcccaaggacaccctgatgatcagccggacccccgaagt |
| | G237A/D265A/P329A/ | gacctgcgtggtggtggccgtgaagcacgaggaccctgaagtg |
| | S267K (GADAPASK) | aagttcaattggtacgtggacggcgtggaagtgcacaacgcca |
| | Nucleic acid | agaccaagcccagagaggaacagtacaacagcacctaccgggt |
| | sequence | ggtgtccgtgctgaccgtgctgcaccaggactggctgaacggc |
| | | aaagagtacaagtgcaaagtctccaacaaggccctggccgccc |
| | | ccatcgagaaaaccatcagcaaggccaagggccagccccgcga |
| | | gccccaggtgtacacactgccccccagccgggacgagctgacc |
| | | aagaaccaggtgtccctgacctgcctggtcaagggcttctacc |
| | | ccagcgatatcgccgtggaatgggagagcaacggccagcccga |
| | | gaacaactacaagaccaccccccctgtgctggacagcgacggc |
| | | tcattcttcctgtacagcaagctgaccgtggacaagtcccggt |
| | | ggcagcagggcaacgtgttcagctgcagcgtgatgcacgaggc |
| | | cctgcacaaccactacacccagaagtccctgagcctgagcccc |
| | | ggcaaa |

TABLE B
Sequences of Antibodies and Fc variants

| SEQ ID | | |
|---|---|---|
| No | Description | Sequence |
| 1268 | FCV-8: Human IgG1 | apellggpsvflfppkpkdtlmisrtpevtcvvvavshedpev |
| | Fc variant | kfnwyvdgvevhnaktkpreeqynstyrvvsvltvlhqdwlng |
| | D265A/P329A | keykckvsnkalaapiektiskakgqprepqvytlppsrdelt |
| | (DAPA) | knqvsltclvkgfypsdiavewesngqpennykttppvldsdg |
| | Amino acid | sfflyskltvdksrwqqgnvfscsvmhealhnhytqkslslsp |
| | sequence | gk |
| 1269 | FCV-8: Human IgG1 | gcccctgaactgctgggcggaccctccgtgttcctgttccccc |
| | Fc variant | caaagcccaaggacaccctgatgatcagccggacccccgaagt |
| | D265A/P329A | gacctgcgtggtggtggccgtgtcccacgaggaccctgaagtg |
| | (DAPA) | aagttcaattggtacgtggacggcgtggaagtgcacaacgcca |
| | Nucleic acid | agaccaagcccagagaggaacagtacaacagcacctaccgggt |
| | sequence | ggtgtccgtgctgaccgtgctgcaccaggactggctgaacggc |
| | | aaagagtacaagtgcaaagtctccaacaaggccctggccgccc |
| | | ccatcgagaaaaccatcagcaaggccaagggccagccccgcga |
| | | gccccaggtgtacacactgccccccagccgggacgagctgacc |
| | | aagaaccaggtgtccctgacctgcctggtcaagggcttctacc |
| | | ccagcgatatcgccgtggaatgggagagcaacggccagcccga |
| | | gaacaactacaagaccaccccccctgtgctggacagcgacggc |
| | | tcattcttcctgtacagcaagctgaccgtggacaagtcccggt |
| | | ggcagcagggcaacgtgttcagctgcagcgtgatgcacgaggc |
| | | cctgcacaaccactacacccagaagtccctgagcctgagcccc |
| | | ggcaaa |
| 1270 | FCV-9: Human IgG1 | apellggpsvflfppkpkdtlmisrtpevtcvvvavshedpev |
| | Fc variant | kfnwyvdgvevhnaktkpreeqyastyrvvsvltvlhqdwlng |
| | D265A/N297A/P329A | keykckvsnkalaapiektiskakgqprepqvytlppsrdelt |
| | (DANAPA) | knqvsltclvkgfypsdiavewesngqpennykttppvldsdg |
| | Amino acid | sfflyskltvdksrwqqgnvfscsvmhealhnhytqkslslsp |
| | sequence | gk |
| 1271 | FCV-9: Human IgG1 | gcccctgaactgctgggaggccctagcgtgttcctgttccccc |
| | Fc variant | caaagcccaaggacaccctgatgatcagccggacccccgaagt |
| | D265A/N297A/P329A | gacctgtgtggtggtggccgtgtctcacgaggaccctgaagtg |
| | (DANAPA) | aagtttaattggtacgtggacggcgtggaagtgcacaacgcca |
| | Nucleic acid | agaccaagcccagagaggaacagtacgccagcacctaccgggt |
| | sequence | ggtgtccgtgctgacagtgctgcaccaggactggctgaacggc |
| | | aaagagtacaagtgcaaggtgtccaacaaggccctggccgctc |
| | | ccatcgagaaaaccatcagcaaggccaagggccagccccgcga |
| | | accccaggtgtacacactgccccctagcagggacgagctgacc |
| | | aagaaccaggtgtccctgacctgcctcgtgaagggcttctacc |
| | | cctccgatatcgccgtggaatgggagagcaacggccagcccga |
| | | gaacaactacaagaccaccccccctgtgctggactccgacggc |
| | | tcattcttcctgtacagcaagctgaccgtggacaagtcccggt |
| | | ggcagcagggcaacgtgttcagctgctccgtgatgcacgaggc |
| | | cctgcacaaccactacacccagaagtccctgagcctgagcccc |
| | | ggcaaa |
| 1272 | Anti-CD3 human | evqlvesggglvqpggslklscaasgftfntyamnwvrqasgk |
| | IgG1 Heavy Chain | glewvgrirskynnyatyyadsvkdrftisrddskstlylqmn |
| | Amino acid | slktedtavyycvrhgnfgnsyvswfaywgqgtlvtvssastk |
| | sequence | gpsvfplapsskstsggtaalgclvkdyfpepvtvswnsgalt |
| | | sgvhtfpavlqssglyslssvvtvpssslgtqtyicnvnhkps |
| | | ntkvdkrvepkscdkthtcppcpapellggpsvflfppkpkdt |
| | | lmisrtpevtcvvvdvshedpevkfnwyvdgvevhnaktkpre |
| | | eqynstyrvvsvltvlhqdwlngkeykckvsnkalpapiekti |
| | | skakgqprepqvytlppsrdeltknqvsltclvkgfypsdiav |
| | | ewesngqpennykttppvldsdgsfflyskltvdksrwqqgnv |
| | | fscsvmhealhnhytqkslslspgk |
| 1273 | Anti-CD3 human | gaagtgcagctggtggaatctggcggcggactggtgcagcctg |
| | IgG1 Heavy Chain | gcggatctctgaagctgagctgtgccgccagcggcttcacctt |
| | Nucleic acid | caacacctacgccatgaactgggtgcgccaggcctctggcaag |
| | sequence | ggcctggaatgggtgggacggatcagaagcaagtacaacaatt |
| | | acgccacctactacgccgacagcgtgaaggaccggttcaccat |
| | | cagccgggacgacagcaagagcaccctgtacctgcagatgaac |
| | | agcctgaaaaccgaggacaccgccgtgtactactgcgtgcggc |
| | | acggcaacttcggcaacagctatgtgtcttggtttgcctactg |
| | | gggccagggcaccctcgtgacagtgagctcagctagcaccaag |
| | | ggccccagcgtgttccccctggcgcccagcagcaagagcacca |
| | | gcggcggcacagccgccctgggctgcctggtgaaggactactt |
| | | ccccgagccagtgaccgtgtcctggaacagcggagccctgacc |
| | | tccggcgtgcacaccttccccgccgtgctgcagagcagcggcc |
| | | tgtacagcctgagcagcgtggtgaccgtgcccagcagcagcct |
| | | gggcacccagacctacatctgcaacgtgaaccacaagcccagc |
| | | aacaccaaggtggacaagagagtggagcccaagagctgcgaca |
| | | agacccacacctgccccccctgtcctgcccctgaactgctggg |
| | | cggaccctccgtgttcctgttccccccaaagcccaaggacacc |
| | | ctgatgatcagccggacccccgaagtgacctgcgtggtggtgg |
| | | acgtgtcccacgaggaccctgaagtgaagttcaattggtacgt |
| | | ggacggcgtggaagtgcacaacgccaagaccaagcccagagag |
| | | gaacagtacaacagcacctaccgggtggtgtccgtgctgaccg |
| | | tgctgcaccaggactggctgaacggcaaagagtacaagtgcaa |
| | | agtctccaacaaggccctgcctgcccccatcgagaaaaccatc |
| | | agcaaggccaagggccagccccgcgagccccaggtgtacacac |
| | | tgccccccagccgggacgagctgaccaagaaccaggtgtccct |
| | | gacctgcctggtcaagggcttctaccccagcgatatcgccgtg |
| | | gaatgggagagcaacggccagcccgagaacaactacaagacca |
| | | ccccccctgtgctggacagcgacggctcattcttcctgtacag |
| | | caagctgaccgtggacaagtcccggtggcagcagggcaacgtg |
| | | ttcagctgcagcgtgatgcacgaggccctgcacaaccactaca |
| | | cccagaagtccctgagcctgagccccggcaaa |
| 1274 | Anti-CD3 human | evqlvesggglvqpggslklscaasgftfntyamnwvrqasgk |
| | IgG1 | glewvgrirskynnyatyyadsvkdrftisrddskstlylqmn |
| | R214K allotype | slktedtavyycvrhgnfgnsyvswfaywgqgtlvtvssastk |
| | Heavy Chain | gpsvfplapsskstsggtaalgclvkdyfpepvtvswnsgalt |
| | Amino acid | sgvhtfpavlqssglyslssvvtvpssslgtqtyicnvnhkps |
| | sequence | ntkvdkkvepkscdkthtcppcpapellggpsvflfppkpkdt |
| | | lmisrtpevtcvvvdvshedpevkfnwyvdgvevhnaktkpre |
| | | eqynstyrvvsvltvlhqdwlngkeykckvsnkalpapiekti |
| | | skakgqprepqvytlppsrdeltknqvsltclvkgfypsdiav |
| | | ewesngqpennykttppvldsdgsfflyskltvdksrwqqgnv |
| | | fscsvmhealhnhytqkslslspgk |
| 1275 | Anti-CD3 human | gaagtgcagctggtggaatctggcggcggactggtgcagcctg |
| | IgG1 | gcggatctctgaagctgagctgtgccgccagcggcttcacctt |
| | R214K allotype | caacacctacgccatgaactgggtgcgccaggcctctggcaag |
| | Heavy Chain | ggcctggaatgggtgggacggatcagaagcaagtacaacaatt |
| | Nucleic acid | acgccacctactacgccgacagcgtgaaggaccggttcaccat |
| | sequence | cagccgggacgacagcaagagcaccctgtacctgcagatgaac |
| | | agcctgaaaaccgaggacaccgccgtgtactactgcgtgcggc |
| | | acggcaacttcggcaacagctatgtgtcttggtttgcctactg |
| | | gggccagggcaccctcgtgacagtgagctcagctagcaccaag |
| | | ggccccagcgtgttccccctggcgcccagcagcaagagcacca |
| | | gcggcggcacagccgccctgggctgcctggtgaaggactactt |
| | | ccccgagccagtgaccgtgtcctggaacagcggagccctgacc |
| | | tccggcgtgcacaccttccccgccgtgctgcagagcagcggcc |
| | | tgtacagcctgagcagcgtggtgaccgtgcccagcagcagcct |
| | | gggcacccagacctacatctgcaacgtgaaccacaagcccagc |
| | | aacaccaaggtggacaagaaggtggagcccaagagctgcgaca |
| | | agacccacacctgccccccctgtcctgcccctgaactgctggg |
| | | cggaccctccgtgttcctgttccccccaaagcccaaggacacc |
| | | ctgatgatcagccggacccccgaagtgacctgcgtggtggtgg |
| | | acgtgtcccacgaggaccctgaagtgaagttcaattggtacgt |
| | | ggacggcgtggaagtgcacaacgccaagaccaagcccagagag |
| | | gaacagtacaacagcacctaccgggtggtgtccgtgctgaccg |
| | | tgctgcaccaggactggctgaacggcaaagagtacaagtgcaa |
| | | agtctccaacaaggccctgcctgcccccatcgagaaaaccatc |
| | | agcaaggccaagggccagccccgcgagccccaggtgtacacac |
| | | tgccccccagccgggacgagctgaccaagaaccaggtgtccct |
| | | gacctgcctggtcaagggcttctaccccagcgatatcgccgtg |
| | | gaatgggagagcaacggccagcccgagaacaactacaagacca |
| | | ccccccctgtgctggacagcgacggctcattcttcctgtacag |
| | | caagctgaccgtggacaagtcccggtggcagcagggcaacgtg |
| | | ttcagctgcagcgtgatgcacgaggccctgcacaaccactaca |
| | | cccagaagtccctgagcctgagccccggcaaa |
| 1276 | Anti-CD3 human | qavvtqepsltvspggtvtltcrsstgavttsnyanwvqqkpg |
| | Light Chain | qaprgliggtnkrapwtparfsgsllgdkaaltlsgaqpedea |
| | Amino acid | eyfcalwysnlwvfgggtkltvlgqpkaapsvtlfppsseelq |
| | sequence | ankatlvclisdfypgavtvawkadsspvkagvetttpskqsn |
| | | nkyaassylsltpeqwkshrsyscqvthegstvektvaptecs |
| 1277 | Anti-CD3 human | caggctgtcgtgacccaggaacctagcctgaccgtgtctcctg |
| | Light Chain | gcggaaccgtgaccctgacctgtagatctagcacaggcgccgt |
| | Nucleic acid | gaccaccagcaactacgccaattgggtgcagcagaagcccggc |
| | sequence | caggctcctagaggactgatcggcggcaccaacaagagagccc |
| | | cttggacccctgccagattcagcggctctctgctgggagataa |
| | | ggccgccctgacactgtctggcgcccagcctgaggatgaggcc |
| | | gagtacttttgcgccctgtggtacagcaacctgtgggtgttcg |
| | | gcggaggcaccaagctgaccgtgctgggccagcctaaggccgc |
| | | tccctccgtgaccctgttcccccccagctccgaggaactgcag |
| | | gccaacaaggccaccctggtgtgcctgatcagcgacttctacc |
| | | ctggcgccgtgaccgtggcctggaaggccgacagcagccccgt |
| | | gaaggccggcgtggagacaaccacccccagcaagcagagcaac |
| | | aacaagtacgccgccagcagctacctgagcctgacccccgagc |
| | | agtggaagagccacagaagctacagctgccaggtcacccacga |
| | | gggcagcaccgtggagaaaaccgtggcccccaccgagtgcagc |

7.2.2.1.1. Fc Domains with Altered FcR Binding

The Fc domains of the CD19 binding molecules can show altered binding to one or more Fc-receptors (FcRs) in comparison with the corresponding native immunoglobulin. The binding to any particular Fc-receptor can be increased or decreased. In one embodiment, the Fc domain comprises one or more modifications which alter its Fc-receptor binding profile.

Human cells can express a number of membrane bound FcRs selected from FcαR, FcεR, FcγR, FcRn and glycan receptors. Some cells are also capable of expressing soluble (ectodomain) FcR (Fridman et al., 1993, J Leukocyte Biology 54: 504-512). FcγR can be further divided by affinity of IgG binding (high/low) and biological effect (activating/inhibiting). Human FcγRI is widely considered to be the sole ‘high affinity’ receptor whilst all of the others are considered as medium to low. FcγRIIb is the sole receptor with ‘inhibitory’ functionality by virtue of its intracellular ITIM motif whilst all of the others are considered as ‘activating’ by virtue of ITAM motifs or pairing with the common FcγR-γ chain. FcγRIIIb is also unique in that although activatory it associates with the cell via a GPI anchor. In total, humans express six “standard” FcγRs: FcγRI, FcγRIIa, FcγRIIb, FcγRIIc, FcγRIIIa, and FcγRIIIb. In addition to these sequences there are a large number of sequence or allotypic variants spread across these families. Some of these have been found to have important functional consequence and so are sometimes considered to be receptor sub-types of their own. Examples include FcγRIIa^{H134R}, FcγRIIb^{I190T}, FcγRIIIa^{F158V}, FcγRIIIb^{NA1}, FcγRIIIb^{NA2}, and FcγRIII^{SH}. Each receptor sequence has been shown to have different affinities for the 4 sub-classes of IgG: IgG1, IgG2, IgG3 and IgG4 (Bruhns, 1993, Blood 113:3716-3725). Other species have somewhat different numbers and functionality of FcγR, with the mouse system being the best studied to date and comprising of 4 FcγR, FcγRI FcγRIIb FcγRIII FcγRIV (Bruhns, 2012, Blood 119:5640-5649). Human FcγRI on cells is normally considered to be “occupied” by monomeric IgG in normal serum conditions due to its affinity for IgG1/IgG3/IgG4 (about 10⁻⁸ M) and the concentration of these IgG in serum (about 10 mg/ml). Hence cells bearing FcγRI on their surface are considered to be capable for “screening” or “sampling” of their antigenic environment vicariously through the bound polyspecific IgG. The other receptors having lower affinities for IgG sub-classes (in the range of about 10⁻⁵-10⁻⁷ M) are normally considered to be “unoccupied.” The low affinity receptors are hence inherently sensitive to the detection of and activation by antibody involved immune complexes. The increased Fc density in an antibody immune complex results in increased functional affinity of binding avidity to low affinity FcγR. This has been demonstrated in vitro using a number of methods (Shields et al., 2001, J Biol Chem 276(9):6591-6604; Lux et al., 2013, J Immunol 190:4315-4323). It has also been implicated as being one of the primary modes of action in the use of anti-RhD to treat ITP in humans (Crow, 2008, Transfusion Medicine Reviews 22:103-116).

Many cell types express multiple types of FcγR and so binding of IgG or antibody immune complex to cells bearing FcγR can have multiple and complex outcomes depending upon the biological context. Most simply, cells can either receive an activatory, inhibitory or mixed signal. This can result in events such as phagocytosis (e.g., macrophages and neutrophils), antigen processing (e.g., dendritic cells), reduced IgG production (e.g., B-cells) or degranulation (e.g., neutrophils, mast cells). There are data to support that the inhibitory signal from FcγRIIb can dominate that of activatory signals (Proulx, 2010, Clinical Immunology 135:422-429).

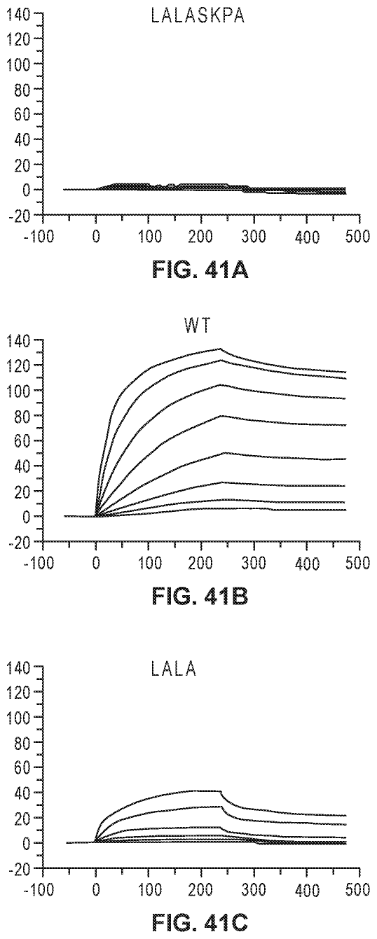

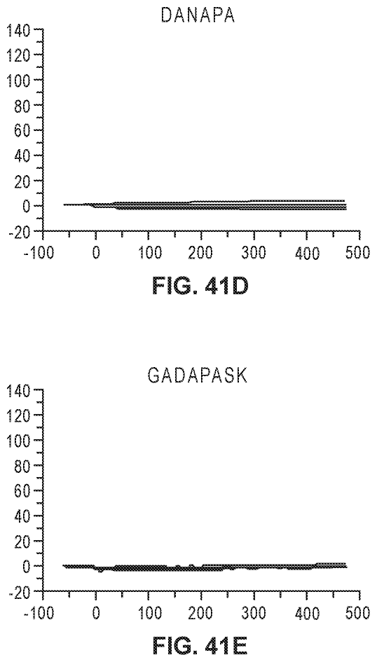

There are a number of useful Fc substitutions that can be made to alter binding to one or more of the FcγR receptors. Substitutions that result in increased binding as well as decreased binding can be useful. For example, it is known that increased binding to FcγRIIIa generally results in increased ADCC (antibody dependent cell-mediated cytotoxicity; the cell-mediated reaction where nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the present disclosure include those listed in US 2006/0024298 (particularly FIG. 41), US 2006/0121032, US 2006/0235208, US 2007/0148170, and US 2019/0100587. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V, 299T, 265A/297A/329A, 265N/297D/329G, and 265E/297Q/329S.

FcRn has a crucial role in maintaining the long half-life of IgG in the serum of adults and children. The receptor binds IgG in acidified vesicles (pH<6.5) protecting the IgG molecule from degradation, and then releasing it at the higher pH of 7.4 in blood.

FcRn is unlike leukocyte Fc receptors, and instead, has structural similarity to MHC class I molecules. It is a heterodimer composed of a β₂-microglobulin chain, non-covalently attached to a membrane-bound chain that includes three extracellular domains. One of these domains, including a carbohydrate chain, together with β₂-microglobulin interacts with a site between the CH2 and CH3 domains of Fc. The interaction includes salt bridges made to histidine residues on IgG that are positively charged at pH<6.5. At higher pH, the His residues lose their positive charges, the FcRn-IgG interaction is weakened and IgG dissociates.

In one embodiment, a CD19 binding molecule comprises an Fc domain that binds to human FcRn.

In one embodiment, the Fc domain has an Fc region(s) (e.g., one or two) comprising a histidine residue at position 310, and in some cases also at position 435. These histidine residues are important for human FcRn binding. In one embodiment, the histidine residues at positions 310 and 435 are native residues, i.e., positions 310 and 435 are not modified. Alternatively, one or both of these histidine residues can be present as a result of a modification.

The CD19 binding molecules can comprise one or more Fc regions that alter Fc binding to FcRn. The altered binding can be increased binding or decreased binding.

In one embodiment, the CD19 binding molecule comprises an Fc domain in which at least one (and optionally both) Fc regions comprises one or more modifications such that it binds to FcRn with greater affinity and avidity than the corresponding native immunoglobulin.

Fc substitutions that increase binding to the FcRn receptor and increase serum half life are described in US 2009/0163699, including, but not limited to, 434S, 434A, 428L, 308F, 2591, 428L/434S, 2591/308F, 4361/428L, 4361 or V/434S, 436V/428L and 2591/308F/428L.

In one embodiment, the Fc region is modified by substituting the threonine residue at position 250 with a glutamine residue (T250Q).

In one embodiment, the Fc region is modified by substituting the methionine residue at position 252 with a tyrosine residue (M252Y)

In one embodiment, the Fc region is modified by substituting the serine residue at position 254 with a threonine residue (S254T).

In one embodiment, the Fc region is modified by substituting the threonine residue at position 256 with a glutamic acid residue (T256E).

In one embodiment, the Fc region is modified by substituting the threonine residue at position 307 with an alanine residue (T307A).

In one embodiment, the Fc region is modified by substituting the threonine residue at position 307 with a proline residue (T307P).

In one embodiment, the Fc region is modified by substituting the valine residue at position 308 with a cysteine residue (V308C).

In one embodiment, the Fc region is modified by substituting the valine residue at position 308 with a phenylalanine residue (V308F).

In one embodiment, the Fc region is modified by substituting the valine residue at position 308 with a proline residue (V308P).

In one embodiment, the Fc region is modified by substituting the glutamine residue at position 311 with an alanine residue (Q311A).

In one embodiment, the Fc region is modified by substituting the glutamine residue at position 311 with an arginine residue (Q311R).

In one embodiment, the Fc region is modified by substituting the methionine residue at position 428 with a leucine residue (M428L).

In one embodiment, the Fc region is modified by substituting the histidine residue at position 433 with a lysine residue (H433K).

In one embodiment, the Fc region is modified by substituting the asparagine residue at position 434 with a phenylalanine residue (N434F).

In one embodiment, the Fc region is modified by substituting the asparagine residue at position 434 with a tyrosine residue (N434Y).

In one embodiment, the Fc region is modified by substituting the methionine residue at position 252 with a tyrosine residue, the serine residue at position 254 with a threonine residue, and the threonine residue at position 256 with a glutamic acid residue (M252Y/S254T/T256E).

In one embodiment, the Fc region is modified by substituting the valine residue at position 308 with a proline residue and the asparagine residue at position 434 with a tyrosine residue (V308P/N434Y).

In one embodiment, the Fc region is modified by substituting the methionine residue at position 252 with a tyrosine residue, the serine residue at position 254 with a threonine residue, the threonine residue at position 256 with a glutamic acid residue, the histidine residue at position 433 with a lysine residue and the asparagine residue at position 434 with a phenylalanine residue (M252Y/S254T/T256E/H433K/N434F).

It will be appreciated that any of the modifications listed above can be combined to alter FcRn binding.

In one embodiment, the CD19 binding molecule comprises an Fc domain in which one or both Fc regions comprise one or more modifications such that the Fc domain binds to FcRn with lower affinity and avidity than the corresponding native immunoglobulin.

In one embodiment, the Fc region comprises any amino acid residue other than histidine at position 310 and/or position 435.

The CD19 binding molecule can comprise an Fc domain in which one or both Fc regions comprise one or more modifications which increase its binding to FcγRIIb. FcγRIIb is the only inhibitory receptor in humans and the only Fc receptor found on B cells.

In one embodiment, the Fc region is modified by substituting the proline residue at position 238 with an aspartic acid residue (P238D).

In one embodiment, the Fc region is modified by substituting the glutamic acid residue at position 258 with an alanine residue (E258A).

In one embodiment, the Fc region is modified by substituting the serine residue at position 267 with an alanine residue (S267A).

In one embodiment, the Fc region is modified by substituting the serine residue at position 267 with a glutamic acid residue (S267E).

In one embodiment, the Fc region is modified by substituting the leucine residue at position 328 with a phenylalanine residue (L328F).

In one embodiment, the Fc region is modified by substituting the glutamic acid residue at position 258 with an alanine residue and the serine residue at position 267 with an alanine residue (E258A/S267A).

In one embodiment, the Fc region is modified by substituting the serine residue at position 267 with a glutamic acid residue and the leucine residue at position 328 with a phenylalanine residue (S267E/L328F).

It will be appreciated that any of the modifications listed above can be combined to increase FcγRIIb binding.

In one embodiment, CD19 binding molecules are provided comprising Fc domains which display decreased binding to FcγR.

In one embodiment, the CD19 binding molecule comprises an Fc domain in which one or both Fc regions comprise one or more modifications that decrease Fc binding to FcγR.

The Fc domain can be derived from IgG1.

In one embodiment, the Fc region is modified by substituting the leucine residue at position 234 with an alanine residue (L234A).

In one embodiment, the Fc region is modified by substituting the leucine residue at position 235 with an alanine residue (L235A).

In one embodiment, the Fc region is modified by substituting the glycine residue at position 236 with an arginine residue (G236R).

In one embodiment, the Fc region is modified by substituting the asparagine residue at position 297 with an alanine residue (N297A) or a glutamine residue (N297Q).

In one embodiment, the Fc region is modified by substituting the serine residue at position 298 with an alanine residue (S298A).

In one embodiment, the Fc region is modified by substituting the leucine residue at position 328 with an arginine residue (L328R).

In one embodiment, the Fc region is modified by substituting the leucine residue at position 234 with an alanine residue and the leucine residue at position 235 with an alanine residue (L234A/L235A).

In one embodiment, the Fc region is modified by substituting the phenylalanine residue at position 234 with an alanine residue and the leucine residue at position 235 with an alanine residue (F234A/L235A).

In one embodiment, the Fc region is modified by substituting the glycine residue at position 236 with an arginine residue and the leucine residue at position 328 with an arginine residue (G236R/L328R).

In one embodiment, the Fc region is modified by substituting the aspartate residue at position 265 with an alanine residue, the asparagine residue at position 297 with an alanine residue and the proline residue at position 329 with an alanine residue (D265A/N297A/P329A).

In one embodiment, the Fc region is modified by substituting the aspartate residue at position 265 with an asparagine residue, the asparagine residue at position 297 with an aspartate residue and the proline residue at position 329 with a glycine residue (D265N/N297D/P329G).

In one embodiment, the Fc region is modified by substituting the aspartate residue at position 265 with a glutamate residue, the asparagine residue at position 297 with an glutamine residue and the proline residue at position 329 with a serine residue (D265E/N297Q/P329S).

It will be appreciated that any of the modifications listed above can be combined to decrease FcγR binding.

In one embodiment, a CD19 binding molecule comprises an Fc domain in which one or both Fc regions comprise one or more modifications that decrease Fc binding to FcγRIIIa without affecting the Fc's binding to FcγRII.

In one embodiment, the Fc region is modified by substituting the serine residue at position 239 with an alanine residue (S239A).

In one embodiment, the Fc region is modified by substituting the glutamic acid residue at position 269 with an alanine residue (E269A).

In one embodiment, the Fc region is modified by substituting the glutamic acid residue at position 293 with an alanine residue (E293A).

In one embodiment, the Fc region is modified by substituting the tyrosine residue at position 296 with a phenylalanine residue (Y296F).

In one embodiment, the Fc region is modified by substituting the valine residue at position 303 with an alanine residue (V303A).

In one embodiment, the Fc region is modified by substituting the alanine residue at position 327 with a glycine residue (A327G).

In one embodiment, the Fc region is modified by substituting the lysine residue at position 338 with an alanine residue (K338A).

In one embodiment, the Fc region is modified by substituting the aspartic acid residue at position 376 with an alanine residue (D376A).

It will be appreciated that any of the modifications listed above can be combined to decrease FcγRIIIa binding.

Fc region variants with decreased FcR binding can be referred to as “FcγR ablation variants,” “FcγR silencing variants” or “Fc knock out (FcKO or KO)” variants. For some therapeutic applications, it is desirable to reduce or remove the normal binding of an Fc domain to one or more or all of the Fcγ receptors (e.g., FcγR1, FcγRIIa, FcγRIIb, FcγRIIIa) to avoid additional mechanisms of action. That is, for example, in many embodiments, particularly in the use of MBMs that bind CD3 monovalently, it is generally desirable to ablate FcγRIIIa binding to eliminate or significantly reduce ADCC activity. In some embodiments, at least one of the Fc regions of the MBMs described herein comprises one or more Fcγ receptor ablation variants. In some embodiments, both of the Fc regions comprise one or more Fcγ receptor ablation variants. These ablation variants are depicted in Table 3, and each can be independently and optionally included or excluded, with some aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G, E233P/L234V/L235A/G236del, D265A/N297A/P329A, D265N/N297D/P329G, and D265E/N297Q/P329S (“del” connotes a deletion, e.g., G236del refers to a deletion of the glycine at position 236). It should be noted that the ablation variants referenced herein ablate FcγR binding but generally not FcRn binding.

TABLE 3
Ablation Variants

| Variant | Variant(s), cont. |
|---|---|
| G236R | P329K |
| S239G | A330L |
| S239K | A330S/P331S |
| S239Q | I332K |
| S239R | I332R |
| V266D | V266D/A327Q |
| S267K | V266D/P329K |
| S267R | S267R/A327Q |
| H268K | S267R/P329K |
| E269R | G236R/L328R |
| 299R | E233P/L234V/L235A/G236del/S239K |
| 299K | E233P/L234V/L235A/G236del/S267K |
| K322A | E233P/L234V/L235A/G236del/S239K/A327G |
| A327G | E233P/L234V/L235A/G236del/S267K/A327G |
| A327L | E233P/L234V/L235A/G236del |
| A327N | S239K/S267K |
| A327Q | 267K/P329K |
| L328E | D265A/N297A/P329A |
| L328R | D265N/N297D/P329G |
| P329A | D265E/N297Q/P329S |
| P329H | |

In some embodiments, the MBMs of the present disclosure comprises a first Fc region and a second Fc region. In some embodiments, the first Fc region and/or the second Fc region can comprise the following mutations: E233P, L234V, L-235A, G236del, and S267K.

The Fc domain of human IgG1 has the highest binding to the Fcγ receptors, and thus ablation variants can be used when the constant domain (or Fc domain) in the backbone of the heterodimeric antibody is IgG1.

Alternatively, or in addition to ablation variants in an IgG1 background, mutations at the glycosylation position 297, e.g., substituting the asparagine residue at position 297 with an alanine residue (N297A) or a glutamine residue (N297Q), can significantly ablate binding to FcγRIIIa, for example. Human IgG2 and IgG4 have naturally reduced binding to the Fcγ receptors, and thus those backbones can be used with or without the ablation variants.

7.2.2.1.2. Fc Domains with Altered Complement Binding

The CD19 binding molecules can comprise an Fc domain in which one or both Fc regions comprises one or more modifications that alter Fc binding to complement. Altered complement binding can be increased binding or decreased binding.

In one embodiment, the Fc region comprises one or more modifications which decrease its binding to C1q. Initiation of the classical complement pathway starts with binding of hexameric C1q protein to the CH2 domain of antigen bound IgG and IgM.

In one embodiment, the CD19 binding molecule comprises an Fc domain in which one or both Fc regions comprises one or more modifications to decrease Fc binding to C1q.

In one embodiment, the Fc region is modified by substituting the leucine residue at position 234 with an alanine residue (L234A).

In one embodiment, the Fc region is modified by substituting the leucine residue at position 235 with an alanine residue (L235A).

In one embodiment, the Fc region is modified by substituting the leucine residue at position 235 with a glutamic acid residue (L235E).

In one embodiment, the Fc region is modified by substituting the glycine residue at position 237 with an alanine residue (G237A).

In one embodiment, the Fc region is modified by substituting the lysine residue at position 322 with an alanine residue (K322A).

In one embodiment, the Fc region is modified by substituting the proline residue at position 331 with an alanine residue (P331A).

In one embodiment, the Fc region is modified by substituting the proline residue at position 331 with a serine residue (P331S).

In one embodiment, a CD19 binding molecule comprises an Fc domain derived from IgG4. IgG4 has a naturally lower complement activation profile than IgG1, but also weaker binding of FcγR. Thus, in one embodiment, the CD19 binding molecule comprises an IgG4 Fc domain and also comprises one or more modifications that increase FcγR binding.

It will be appreciated that any of the modifications listed above can be combined to reduce C1q binding.

7.2.2.1.3. Fc Domains with Altered Disulfide Architecture

The CD19 binding molecule can include an Fc domain comprising one or more modifications to create and/or remove a cysteine residue. Cysteine residues have an important role in the spontaneous assembly of Fc-based multispecific binding molecules, by forming disulfide bridges between individual pairs of polypeptide monomers. Thus, by altering the number and/or position of cysteine residues, it is possible to modify the structure of the CD19 binding molecule to produce a protein with improved therapeutic properties.

A CD19 binding molecule of the present disclosure can comprise an Fc domain in which one or both Fc regions, e.g., both Fc regions, comprise a cysteine residue at position 309. In one embodiment, the cysteine residue at position 309 is created by a modification, e.g., for an Fc domain derived from IgG1, the leucine residue at position 309 is substituted with a cysteine residue (L309C), for an Fc domain derived from IgG2, the valine residue at position 309 is substituted with a cysteine residue (V309C).

In one embodiment, the Fc region is modified by substituting the valine residue at position 308 with a cysteine residue (V308C).

In one embodiment, two disulfide bonds in the hinge region are removed by mutating a core hinge sequence CPPC (SEQ ID NO: 1179) to SPPS (SEQ ID NO: 1180).

7.2.2.1.4. Fc Domains with Altered Glycosylation

In certain aspects, CD19 binding molecules with improved manufacturability are provided that comprise fewer glycosylation sites than a corresponding immunoglobulin. These proteins have less complex post translational glycosylation patterns and are thus simpler and less expensive to manufacture.

In one embodiment a glycosylation site in the CH2 domain is removed by substituting the asparagine residue at position 297 with an alanine residue (N297A) or a glutamine residue (N297Q). In addition to improved manufacturability, these aglycosyl mutants also reduce FcγR binding as described herein above.

In some embodiments, a CD19 binding molecule can be made that has an altered type of glycosylation, such as a hypofucosylated antibody having reduced amounts of fucosyl residues or an antibody having increased bisecting GlcNac structures. Such altered glycosylation patterns have been demonstrated to increase the ADCC ability of antibodies. Such carbohydrate modifications can be accomplished by, for example, expressing a CD19 binding molecule in a host cell with altered glycosylation machinery. Cells with altered glycosylation machinery have been described in the art and can be used as host cells in which to express CD19 binding molecules to thereby produce CD19 binding molecules with altered glycosylation. For example, EP 1,176,195 by Hang et al. describes a cell line with a functionally disrupted FUT8 gene, which encodes a fucosyl transferase, such that antibodies expressed in such a cell line exhibit hypofucosylation. PCT Publication WO 03/035835 by Presta describes a variant CHO cell line, Lecl3 cells, with reduced ability to attach fucose to Asn(297)-linked carbohydrates, also resulting in hypofucosylation of antibodies expressed in that host cell (see also Shields et al., 2002, J. Biol. Chem. 277:26733-26740). PCT Publication WO 99/54342 by Umana et al. describes cell lines engineered to express glycoprotein-modifying glycosyl transferases (e.g., beta(1,4)—N acetylglucosaminyltransferase III (GnTII)) such that antibodies expressed in the engineered cell lines exhibit increased bisecting GlcNac structures which results in increased ADCC activity of the antibodies (see also Umana et al., Nat. Biotech. 17:176-180, 1999).

### 7.2.2.1.5. Fc Heterodimerization

Many multispecific molecule formats entail dimerization between two Fc regions that, unlike a native immunoglobulin, are operably linked to non-identical antigen-binding domains (or portions thereof, e.g., a VH or VH—CH1 of a Fab). Inadequate heterodimerization of two Fc regions to form an Fc domain has always been an obstacle for increasing the yield of desired multispecific molecules and represents challenges for purification. A variety of approaches available in the art can be used in for enhancing dimerization of Fc regions that might be present in the CD19 binding molecules (and particularly in the MBMs of the disclosure), for example as disclosed in EP 1870459A1; U.S. Pat. Nos. 5,582,996; 5,731,168; 5,910,573; 5,932,448; 6,833,441; 7,183,076; U.S. Patent Application Publication No. 2006204493A1; and PCT Publication No. WO2009/089004A1.

The present disclosure provides CD19 binding molecules comprising Fc heterodimers, i.e., Fc domains comprising heterologous, non-identical Fc regions. Heterodimerization strategies are used to enhance dimerization of Fc regions operably linked to different ABMs (or portions thereof, e.g., a VH or VH—CH1 of a Fab) and reduce dimerization of Fc regions operably linked to the same ABM or portion thereof. Typically, each Fc region in the Fc heterodimer comprises a CH3 domain of an antibody. The CH3 domains are derived from the constant region of an antibody of any isotype, class or subclass, and in some cases, of IgG (IgG1, IgG2, IgG3 and IgG4) class, as described in the preceding section.

Typically, the MBMs comprise other antibody fragments in addition to CH3 domains, such as, CH1 domains, CH2 domains, hinge domain, VH domain(s), VL domain(s), CDR(s), and/or antigen-binding fragments described herein. In some embodiments, the two hetero-polypeptides are two heavy chains forming a bispecific or multispecific molecules. Heterodimerization of the two different heavy chains at CH3 domains give rise to the desired antibody or antibody-like molecule, while homodimerization of identical heavy chains will reduce yield of the desired antibody or molecule. In an exemplary embodiment, the two or more hetero-polypeptide chains comprise two chains comprising CH3 domains and forming the molecules of any of the multispecific molecule formats described above of the present disclosure. In an embodiment, the two hetero-polypeptide chains comprising CH3 domains comprise modifications that favor heterodimeric association of the polypeptides, relative to unmodified chains. Various examples of modification strategies are provided below in Table 4 and subsections (a) to (g) of Section 7.2.2.1.5.

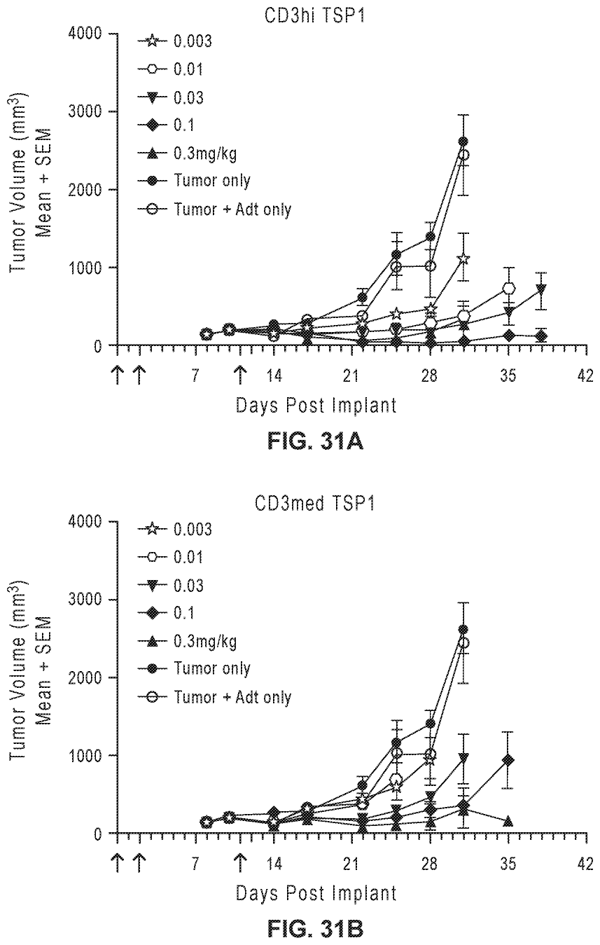

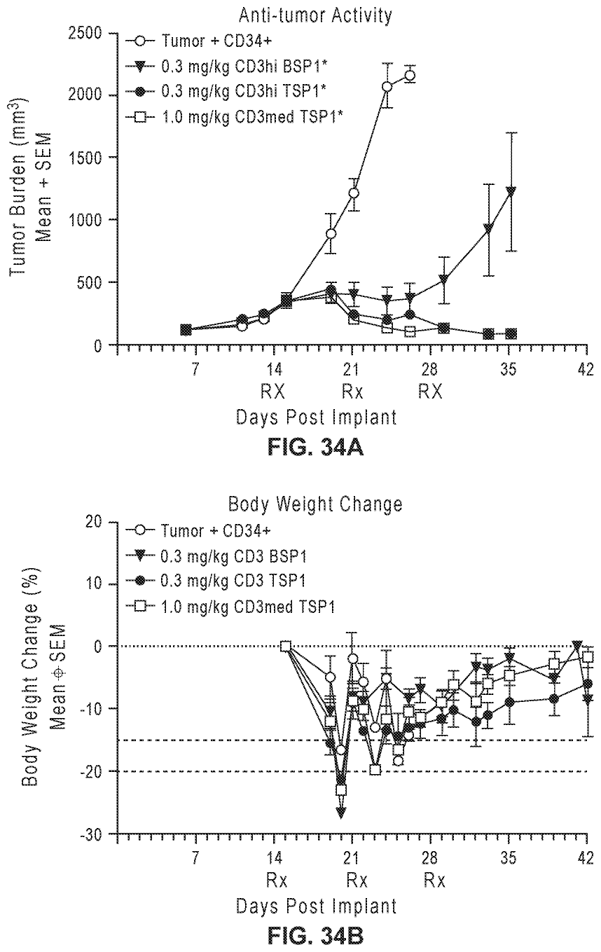

TABLE 4
Fc Heterodimerization Strategies

| NO. | STRATEGY | CH3 DOMAIN 1 | CH3 DOMAIN 2 | REFERENCES |
|---|---|---|---|---|
| Fc 1 | knobs-into-holes | T366Y | Y407T | Ridgway et al., 1996, |
| | (Y-T) | | | Protein Eng 9: 617- |
| | | | | 21 |
| Fc 2 | knobs-into-holes | S354C, T366W | Y349C, T366S, | Atwell et al., 1997, J |
| | (CW-CSAV) | | L368A, Y407V | Mol Biol. 270(1): 26- |
| | | | | 35; Merchant et al., |
| | | | | 1998, Nat Biotechnol |
| | | | | 16: 677-681 |
| Fc 3 | HA-TF | S364H, F405A | Y349T, T394F | Moore et al., 2011, |
| | | | | MAbs 3(6): 546-57 |
| Fc 4 | ZW1 (VYAV- | T350V, L351Y, | T350V, T366L, | Von Kreudenstein et |
| | VLLW) | F405A, Y407V | K392L, T394W | al., 2013, MAbs |
| | | | | 5: 646-54 |
| Fc 5 | CH3 charge pairs | K392D, K409D | E356K, D399K | Gunasekaran et al., |
| | (DD-KK) | | | 2010, J Biol Chem |
| | | | | 285: 19637-46 |
| Fc 6 | IgG1 hingE, CH3 | IgG1: D221E, | IgG1: D221R, | Strop et al., 2012, J |
| | charge pairs (EEE- | P228E, L368E | P228R, K409R | Mol Biol 420: 204-19 |
| | RRR) | | | |
| Fc 7 | IgG2 hingE, CH3 | IgG2: C223E, | IgG2: C223R, | Strop et al., 2012, J |
| | charge pairs (EEE- | P228E, L368E | E225R, P228R, | Mol Biol 420: 204-19 |
| | RRRR) | | K409R | |
| Fc 8 | EW-RVT | K360E, K409W, | Q347R, D399V, | Choi et al., 2013, |
| | | | F405T | Mol Cancer Ther |
| | | | | 12: 2748-59 |
| Fc 9 | EW-RVTS-S | K360E, K409W, | Q347R, D399V, | Choi et al., 2015, |
| | | Y349C | F405T, S354C | Mol Immunol |
| | | | | 65: 377-83 |
| Fc 10 | Biclonic | 366K (+351K) | 351D or E or D at | Geuijen et al., 2014, |
| | | | 349, 368, 349, or | Journal of Clinical |
| | | | 349 + 355 | Oncology |
| | | | | 32: suppl: 560 |
| Fc 11 | DuoBody (L-R) | F405L | K409R | Labrijn et al., 2013, |
| | | | | Proc Natl Acad Sci |
| | | | | USA 110: 5145-50 |
| Fc 12 | SEEDbody | IgG/A chimera | IgG/A chimera | Davis et al., 2010, |
| | | | | Protein Eng Des Sel |
| | | | | 23: 195-202 |
| Fc 13 | BEAT | residues from | residues from | Moretti et al., 2013, |
| | | TCRα interface | TCRβ interface | BMC Proceedings |
| | | | | 7(Suppl 6): O9 |
| Fc 14 | 7.8.60 (DMA- | K360D, D399M, | E345R, Q347R, | Leaver-Fey et al., |
| | RRVV) | Y407A | T366V, K409V | Structure 24: 641-51 |
| Fc 15 | 20.8.34 (SYMV- | Y349S, K370Y, | E356G, E357D, | Leaver-Fey et al., |
| | GDQA) | T366M, K409V | S364Q, Y407A | Structure 24: 641-51 |
| Fc 16 | Skew variant | None | None | FIG. 34 of US |
| | 12757 | | | 2016/0355600 |
| Fc 17 | Skew variant | L368D, K370S | S364K | FIG. 34 of US |
| | 12758 | | | 2016/0355600 |
| Fc 18 | Skew variant | L368D, K370S | S364K, E357L | FIG. 34 of US |
| | 12759 | | | 2016/0355600 |
| Fc 19 | Skew variant | L368D, K370S | S364K, E357Q | FIG. 34 of US |
| | 12760 | | | 2016/0355600 |
| Fc 20 | Skew variant | T411E, K360E, | D401K | FIG. 34 of US |
| | 12761 | Q362E | | 2016/0355600 |
| Fc 21 | Skew variant | L368E, K370S | S364K | FIG. 34 of US |
| | 12496 | | | 2016/0355600 |
| Fc 22 | Skew variant | K370S | S364K | FIG. 34 of US |
| | 12511 | | | 2016/0355600 |
| Fc 23 | Skew variant | L368E, K370S | S364K, E357Q | FIG. 34 of US |
| | 12840 | | | 2016/0355600 |
| Fc 24 | Skew variant | K370S | S364K, E357Q | FIG. 34 of US |
| | 12841 | | | 2016/0355600 |
| Fc 25 | Skew variant | L368E, K370S | S364K | FIG. 34 of US |
| | 12894 | | | 2016/0355600 |
| Fc 26 | Skew variant | K370S | S364K | FIG. 34 of US |
| | 12895 | | | 2016/0355600 |
| Fc 27 | Skew variant | L368E, K370S | S364K, E357Q | FIG. 34 of US |
| | 12896 | | | 2016/0355600 |
| Fc 28 | Skew variant | K370S | S364K, E357Q | FIG. 34 of US |
| | 12901 | | | 2016/0355600 |
| Fc 29 | pI_ISO(−) | I199T, N203D, | | FIG. 31 of US |
| | | K274Q, R355Q, | | 2016/0355600 |
| | | N384S, K392N, | | |
| | | V397M, Q419E, | | |
| | | DEL447 | | |
| Fc 30 | pI_(−)_Isosteric_A | N208D, Q295E, | | FIG. 31 of US |
| | | N384D, Q418E, | | 2016/0355600 |
| | | N421D | | |
| Fc 31 | pI_(−)_isosteric_B | N208D, Q295E, | | FIG. 31 of US |
| | | Q418E, N421D | | 2016/0355600 |
| Fc 32 | pI_ISO(+RR) | Q196K, I199T, | | FIG. 31 of US |
| | | P217R, P228R, | | 2016/0355600 |
| | | N276K | | |
| Fc 33 | pI_ISO(+) | Q196K, I199T, | | FIG. 31 of US |
| | | N276K | | 2016/0355600 |
| Fc 34 | pI_(+) isosteric_A | E269Q, E272Q, | | FIG. 31 of US |
| | | E283Q, E357Q, | | 2016/0355600 |
| Fc 35 | pI_(+)_isosteric_B | E269Q, E272Q, | | FIG. 31 of US |
| | | E283Q | | 2016/0355600 |
| Fc 36 | pI_(+) | E269Q, E272Q | | FIG. 31 of US |
| | isosteric_E269Q, | | | 2016/0355600 |
| | E272Q | | | |
| Fc 37 | pI_(+)_isosteric_E2 | E269Q, E283Q | | FIG. 31 of US |
| | 69Q, E283Q | | | 2016/0355600 |
| Fc 38 | pI_(+) | E272Q, E283Q | | FIG. 31 of US |
| | isosteric_E2720, | | | 2016/0355600 |
| | E283Q | | | |
| Fc 39 | pI_(+)_isosteric_E2 | E269Q | | FIG. 31 of US |
| | 69Q | | | 2016/0355600 |
| Fc 40 | Heterodimerization | F405A | T394F | FIG. 30A of US |
| | | | | 2016/0355600 |
| Fc 41 | Heterodimerization | S364D | Y349K | FIG. 30A of US |
| | | | | 2016/0355600 |
| Fc 42 | Heterodimerization | S364E | L368K | FIG. 30A of US |
| | | | | 2016/0355600 |
| Fc 43 | Heterodimerization | S364E | Y349K | FIG. 30A of US |
| | | | | 2016/0355600 |
| Fc 44 | Heterodimerization | S364F | K370G | FIG. 30A of US |
| | | | | 2016/0355600 |
| Fc 45 | Heterodimerization | S364H | Y349K | FIG. 30A of US |
| | | | | 2016/0355600 |
| Fc 46 | Heterodimerization | S364H | Y349T | FIG. 30A of US |
| | | | | 2016/0355600 |
| Fc 47 | Heterodimerization | S364Y | K370G | FIG. 30A of US |
| | | | | 2016/0355600 |
| Fc 48 | Heterodimerization | T411K | K370E | FIG. 30A of US |
| | | | | 2016/0355600 |
| Fc 49 | Heterodimerization | V397S, F405A | T394F | FIG. 30A of US |
| | | | | 2016/0355600 |
| Fc 50 | Heterodimerization | K370R, T411K | K370E, T411E | FIG. 30A of US |
| | | | | 2016/0355600 |
| Fc 51 | Heterodimerization | L351E, S364D | Y349K, L351K | FIG. 30A of US |
| | | | | 2016/0355600 |
| Fc 52 | Heterodimerization | L351E, S364E | Y349K, L351K | FIG. 30A of US |
| | | | | 2016/0355600 |
| Fc 53 | Heterodimerization | L351E, T366D | L351K, T366K | FIG. 30A of US |
| | | | | 2016/0355600 |
| Fc 54 | Heterodimerization | P395T, V397S, | T394F | FIG. 30A of US |
| | | F405A | | 2016/0355600 |
| Fc 55 | Heterodimerization | S364D, K370G | S364Y, K370R | FIG. 30A of US |
| | | | | 2016/0355600 |
| Fc 56 | Heterodimerization | S364D, T394F | Y349K, F405A | FIG. 30A of US |
| | | | | 2016/0355600 |
| Fc 57 | Heterodimerization | S364E, F405A | Y349K, T394F | FIG. 30A of US |
| | | | | 2016/0355600 |
| Fc 58 | Heterodimerization | S364E, F405S | Y349K, T394Y | FIG. 30A of US |
| | | | | 2016/0355600 |
| Fc 59 | Heterodimerization | S364E, T411E | Y349K, D401K | FIG. 30A of US |
| | | | | 2016/0355600 |
| Fc 60 | Heterodimerization | S364H, D401K | Y349T, T411E | FIG. 30A of US |
| | | | | 2016/0355600 |
| Fc 61 | Heterodimerization | S364H, F405A | Y349T, T394F | FIG. 30A of US |
| | | | | 2016/0355600 |
| Fc 62 | Heterodimerization | S364H, T394F | Y349T, F405A | FIG. 30A of US |
| | | | | 2016/0355600 |
| Fc 63 | Heterodimerization | Y349C, S364E | Y349K, S354C | FIG. 30A of US |
| | | | | 2016/0355600 |
| Fc 64 | Heterodimerization | L351E, S364D, | Y349K, L351K, | FIG. 30A of US |
| | | F405A | T394F | 2016/0355600 |
| Fc 65 | Heterodimerization | L351K, S364H, | Y349T, L351E, | FIG. 30A of US |
| | | D401K | T411E | 2016/0355600 |
| Fc 66 | Heterodimerization | S364E, T411E, | Y349K, T394F, | FIG. 30A of US |
| | | F405A | D401K | 2016/0355600 |
| Fc 67 | Heterodimerization | S364H, D401K, | Y349T, T394F, | FIG. 30A of US |
| | | F405A | T411E | 2016/0355600 |
| Fc 68 | Heterodimerization | S364H, F405A, | Y349T, T394F, | FIG. 30A of US |
| | | T411E | D401K | 2016/0355600 |
| Fc 69 | Heterodimerization | T411E, K360E, | D401K | FIG. 30C of US |
| | | N390D | | 2016/0355600 |
| Fc 70 | Heterodimerization | T411E, Q362E, | D401K | FIG. 30C of US |
| | | N390D | | 2016/0355600 |
| Fc 71 | Heterodimerization | T411E, Q347R | D401K, K360D | FIG. 30C of US |
| | | | | 2016/0355600 |
| Fc 72 | Heterodimerization | T411E, Q347R | D401K, K360E | FIG. 30C of US |
| | | | | 2016/0355600 |
| Fc 73 | Heterodimerization | T411E, K360 | D401K, Q347K | FIG. 30C of US |
| | | | | 2016/0355600 |
| Fc 74 | Heterodimerization | T411E, K360D | D401K, Q347R | FIG. 30C of US |
| | | | | 2016/0355600 |
| Fc 75 | Heterodimerization | T411E, K360E | D401K, Q347K | FIG. 30C of US |
| | | | | 2016/0355600 |
| Fc 76 | Heterodimerization | T411E, K360E | D401K, Q347R | FIG. 30C of US |
| | | | | 2016/0355600 |
| Fc 77 | Heterodimerization | T411E, S364K | D401K, K370S | FIG. 30C of US |
| | | | | 2016/0355600 |
| Fc 78 | Heterodimerization | T411E, K370S | D401K, S364K | FIG. 30C of US |
| | | | | 2016/0355600 |
| Fc 79 | Heterodimerization | Q347E | E357Q | FIG. 30C of US |
| | | | | 2016/0355600 |
| Fc 80 | Heterodimerization | Q347E | E357Q, Q362K | FIG. 30C of US |
| | | | | 2016/0355600 |
| Fc 81 | Heterodimerization | K360D, Q362E | Q347R | FIG. 30C of US |
| | | | | 2016/0355600 |
| Fc 82 | Heterodimerization | K360D, Q362E | D401K | FIG. 30C of US |
| | | | | 2016/0355600 |
| Fc 83 | Heterodimerization | K360D, Q362E | Q347R, D401K | FIG. 30C of US |
| | | | | 2016/0355600 |
| Fc 84 | Heterodimerization | K360E, Q362E | Q347R | FIG. 30C of US |
| | | | | 2016/0355600 |
| Fc 85 | Heterodimerization | K360E, Q362E | D401K | FIG. 30C of US |
| | | | | 2016/0355600 |
| Fc 86 | Heterodimerization | K360E, Q362E | Q347R, D401K | FIG. 30C of US |
| | | | | 2016/0355600 |
| Fc 87 | Heterodimerization | Q362E, N390D | D401K | FIG. 30C of US |
| | | | | 2016/0355600 |
| Fc 88 | Heterodimerization | Q347E, K360D | D401N | FIG. 30C of US |
| | | | | 2016/0355600 |
| Fc 89 | Heterodimerization | K360D | Q347R, N390K | FIG. 30C of US |
| | | | | 2016/0355600 |
| Fc 90 | Heterodimerization | K360D | N390K, D401N | FIG. 30C of US |
| | | | | 2016/0355600 |
| Fc 91 | Heterodimerization | K360E | Y349H | FIG. 30C of US |
| | | | | 2016/0355600 |
| Fc 92 | Heterodimerization | K370S, Q347E | S364K | FIG. 30C of US |
| | | | | 2016/0355600 |
| Fc 93 | Heterodimerization | K370S, E357L | S364K | FIG. 30C of US |
| | | | | 2016/0355600 |
| Fc 94 | Heterodimerization | K370S, E357Q | S364K | FIG. 30C of US |
| | | | | 2016/0355600 |
| Fc 95 | Heterodimerization | K370S, Q347E, | S364K | FIG. 30C of US |
| | | E357L | | 2016/0355600 |
| Fc 96 | Heterodimerization | K370S, Q347E, | S364K | FIG. 30C of US |
| | | E357Q | | 2016/0355600 |
| Fc 97 | Heterodimerization | L368D, K370S, | S364K | FIG. 30D of US |
| | | Q347E | | 2016/0355600 |
| Fc 98 | Heterodimerization | L368D, K370S, | S364K | FIG. 30D of US |
| | | E357L | | 2016/0355600 |
| Fc 99 | Heterodimerization | L368D, K370S, | S364K | FIG. 30D of US |
| | | E357Q | | 2016/0355600 |
| Fc 100 | Heterodimerization | L368D, K370S, | S364K | FIG. 30D of US |
| | | Q347E, E357L | | 2016/0355600 |
| Fc 101 | Heterodimerization | L368D, K370S, | S364K | FIG. 30D of US |
| | | Q347E, E357Q | | 2016/0355600 |
| Fc 102 | Heterodimerization | L368E, K370S, | S364K | FIG. 30D of US |
| | | Q347E | | 2016/0355600 |
| Fc 103 | Heterodimerization | L368E, K370S, | S364K | FIG. 30D of US |
| | | E357L | | 2016/0355600 |
| Fc 104 | Heterodimerization | L368E, K370S, | S364K | FIG. 30D of US |
| | | E357Q | | 2016/0355600 |
| Fc 105 | Heterodimerization | L368E, K370S, | S364K | FIG. 30D of US |
| | | Q347E, E357L | | 2016/0355600 |
| Fc 106 | Heterodimerization | L368E, K370S, | S364K | FIG. 30D of US |
| | | Q347E, E357Q | | 2016/0355600 |
| Fc 107 | Heterodimerization | L368D, K370T, | S364K | FIG. 30D of US |
| | | Q347E | | 2016/0355600 |
| Fc 108 | Heterodimerization | L368D, K370T, | S364K | FIG. 30D of US |
| | | E357L | | 2016/0355600 |
| Fc 109 | Heterodimerization | L368D, K370T, | S364K | FIG. 30D of US |
| | | E357Q | | 2016/0355600 |
| Fc 110 | Heterodimerization | L368D, K370T, | S364K | FIG. 30D of US |
| | | Q347E, E357L | | 2016/0355600 |
| Fc 111 | Heterodimerization | L368D, K370T, | S364K | FIG. 30D of US |
| | | Q347E, E357Q | | 2016/0355600 |
| Fc 112 | Heterodimerization | L368E, K370T, | S364K | FIG. 30D of US |
| | | Q347E | | 2016/0355600 |
| Fc 113 | Heterodimerization | L368E, K370T, | S364K | FIG. 30D of US |
| | | E357L | | 2016/0355600 |
| Fc 114 | Heterodimerization | L368E, K370T, | S364K | FIG. 30D of US |
| | | E357Q | | 2016/0355600 |
| Fc 115 | Heterodimerization | L368E, K370T, | S364K | FIG. 30D of US |
| | | Q347E, E357L | | 2016/0355600 |
| Fc 116 | Heterodimerization | L368E, K370T, | S364K | FIG. 30D of US |
| | | Q347E, E357Q | | 2016/0355600 |
| Fc 117 | Heterodimerization | T411E, Q362E | D401K, T411K | FIG. 30D of US |
| | | | | 2016/0355600 |
| Fc 118 | Heterodimerization | T411E, N390D | D401K, T411K | FIG. 30D of US |
| | | | | 2016/0355600 |
| Fc 119 | Heterodimerization | T411E, Q362E | D401R, T411R | FIG. 30D of US |
| | | | | 2016/0355600 |
| Fc 120 | Heterodimerization | T411E, N390D | D401R, T411R | FIG. 30D of US |
| | | | | 2016/0355600 |
| Fc 121 | Heterodimerization | Y407T | T366Y | FIG. 30D of US |
| | | | | 2016/0355600 |
| Fc 122 | Heterodimerization | F405A | T394W | FIG. 30D of US |
| | | | | 2016/0355600 |
| Fc 123 | Heterodimerization | T366Y, F405A | T394W, Y407T | FIG. 30D of US |
| | | | | 2016/0355600 |
| Fc 124 | Heterodimerization | T366S, L368A, | T366W | FIG. 30D of US |
| | | Y407V | | 2016/0355600 |
| Fc 125 | Heterodimerization | T366S, L368A, | T366W, S354C | FIG. 30D of US |
| | | Y407V, Y349C | | 2016/0355600 |
| Fc 126 | Heterodimerization | K392D, K409D | E356K, D399K | FIG. 30E of US |
| | | | | 2016/0355600 |
| Fc 127 | Heterodimerization | K370D, K392D, | E356K, E357K, | FIG. 30E of US |
| | | K409D | D399K | 2016/0355600 |
| Fc 128 | Heterodimerization | I199T, N203D, | Q196K, L99T, | FIG. 30E of US |
| | | K247Q, R355Q, | P217R, P228R, | 2016/0355600 |
| | | N384S, K392N, | N276K | |
| | | V397M, Q419E, | | |
| | | K447 | | |
| Fc 129 | Heterodimerization | I199T, N203D, | Q196K, L99T, | FIG. 30E of US |
| | | K247Q, R355Q, | N276K | 2016/0355600 |
| | | N384S, K392N, | | |
| | | V397M, Q419E, | | |
| | | K447 | | |
| Fc 130 | Heterodimerization | N384S, K392N, | N276K | FIG. 30E of US |
| | | V397M, Q419E | | 2016/0355600 |
| Fc 131 | Heterodimerization | D221E, P228E, | D221R, P228R, | FIG. 30E of US |
| | | L368E | K409R | 2016/0355600 |
| Fc 132 | Heterodimerization | C220E, P228E, | C220R, E224R, | FIG. 30E of US |
| | | L368E | P228R, K409R | 2016/0355600 |
| Fc 133 | Heterodimerization | F405L | K409R | FIG. 30E of US |
| | | | | 2016/0355600 |
| Fc 134 | Heterodimerization | T366I, K392M, | F405A, Y407V | FIG. 30E of US |
| | | T394W | | 2016/0355600 |
| Fc 135 | Heterodimerization | T366V, K409F | L351Y, Y407A | FIG. 30E of US |
| | | | | 2016/0355600 |
| Fc 136 | Heterodimerization | T366A, K392E, | D399R, S400R, | FIG. 30E of US |
| | | K409F, T411E | Y407A | 2016/0355600 |
| Fc 137 | Heterodimerization | L351K | L351E | FIG. 30E of US |
| | | | | 2016/0355600 |
| Fc 138 | Heterodimerization | I199T, N203D, | Q196K, L199T, | FIG. 30E of US |
| | | K247Q, R355Q, | P217R, P228R, | 2016/0355600 |
| | | Q419E, K447 | N276K | |
| Fc 139 | Heterodimerization | I199T, N203D, | Q196K, I199T, | FIG. 30E of US |
| | | K247Q, R355Q, | N276K | 2016/0355600 |
| | | Q419E, K447 | | |
| Fc 140 | Heterodimerization | I199T, N203D, | | FIG. 30E of US |
| | | K274Q, R355Q, | | 2016/0355600 |
| | | N384S, K392N, | | |
| | | V397M, Q419E | | |
| | | DEL447 | | |
| Fc 141 | Heterodimerization | N208D, Q295E | | FIG. 30E of US |
| | | N384D, Q418E | | 2016/0355600 |
| | | N421D | | |
| Fc 142 | Heterodimerization | N208D, Q295E | | FIG. 30E of US |
| | | Q418E, N421D | | 2016/0355600 |
| Fc 143 | Heterodimerization | Q196K, I199T | | FIG. 30E of US |
| | | P217R, P228R | | 2016/0355600 |
| | | N276K | | |
| Fc 144 | Heterodimerization | Q196K, I199T | | FIG. 30E of US |
| | | N276K | | 2016/0355600 |
| Fc 145 | Heterodimerization | E269Q, E272Q | | FIG. 30E of US |
| | | E283Q, E357Q | | 2016/0355600 |
| Fc 146 | Heterodimerization | E269Q, E272Q | | FIG. 30E of US |
| | | E283Q, | | 2016/0355600 |
| Fc 147 | Heterodimerization | E269Q, E272Q | | FIG. 30E of US |
| | | | | 2016/0355600 |
| Fc 148 | Heterodimerization | E269Q, E283Q | | FIG. 30E of US |
| | | | | 2016/0355600 |
| Fc 149 | Heterodimerization | E272Q, E283Q | | FIG. 30E of US |
| | | | | 2016/0355600 |
| Fc 150 | Heterodimerization | E269Q | | FIG. 30E of US |
| | | | | 2016/0355600 |

Exemplary pairs of heterologous, non-identical Fc sequences that can pair to form a Fc heterodimer, and which can be included in CD19 binding molecules of the disclosure, include (i) SEQ ID NO:252 and SEQ ID NO:253, and (ii) SEQ ID NO:252 and SEQ ID NO:254.

(SEQ ID NO: 252)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVCTLPPSREEMTKNQVSLSC
AVKGFYPSDIAVEWESNGQPENNYKTTPPVLDSDGSFFLVSKLTVDKSR
WQQGNVFSCSVMHEALHNHYTQKSLSLSPGK
(SEQ ID NO: 253)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSR
WQQGNVFSCSVMHEALHNHYTQKSLSLSPGK
(SEQ ID NO: 254)
DKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKE
YKCKVSNKALPAPIEKTISKAKGQPREPQVYTLPPCREEMTKNQVSLWC
LVKGFYPSDIAVEWESNGQPENNYKTTPPVLDSDGSFFLYSKLTVDKSR
WQQGNVFSCSVMHEALHNRYTQKSLSLSPGK

An Fc region having an amino acid sequence of one of SEQ ID NOS: 252-254 can be modified to include one or more of the substitutions described in Section 7.2.2.1 (including its subparts), for example to include the substitution(s) corresponding to an ablation variant set forth in Table 3. In some embodiments, a CD19 binding molecule comprises an Fc region having an amino acid sequence of one of SEQ ID NOs:252-254 with a mutation at 1, 2, 3, 4, 5, 6, or more than 6 of positions 233, 234, 235, 236, 237, 239, 265, 266, 267, 268, 269, 297, 299, 322, 327, 328, 329, 330, 331 and 332 (EU numbering), for example mutation(s) described in Section 7.2.2.1 (including its subparts). For example, a CD19 binding molecule can comprise an Fc region having an amino acid sequence of SEQ ID NO:252 with a mutation at 1, 2, 3, 4, 5, 6, or more than 6 of positions 233, 234, 235, 236, 237, 239, 265, 266, 267, 268, 269, 297, 299, 322, 327, 328, 329, 330, 331 and 332 and/or an Fc region having an amino acid sequence of SEQ ID NO:253 with a mutation at 1, 2, 3, 4, 5, 6, or more than 6 of positions 233, 234, 235, 236, 237, 239, 265, 266, 267, 268, 269, 297, 299, 322, 327, 328, 329, 330, 331 and 332 and/or an Fc region having an amino acid sequence of SEQ ID NO:254 with a mutation at 1, 2, 3, 4, 5, 6, or more than 6 of positions 233, 234, 235, 236, 237, 239, 265, 266, 267, 268, 269, 297, 299, 322, 327, 328, 329, 330, 331 and 332.

### (a) Steric Variants

CD19 binding molecules can comprise one or more, e.g., a plurality, of modifications to one or more of the constant domains of an Fc domain, e.g., to the CH3 domains. In one example, a CD19 binding molecule of the present disclosure comprises two polypeptides that each comprise a heavy chain constant domain of an antibody, e.g., a CH2 or CH3 domain. In an example, the two heavy chain constant domains, e.g., the CH2 or CH3 domains of the CD19 binding molecule comprise one or more modifications that allow for a heterodimeric association between the two chains. In one aspect, the one or more modifications are disposed on CH2 domains of the two heavy chains. In one aspect, the one or more modifications are disposed on CH3 domains of at least two polypeptides of the CD19 binding molecule.

One mechanism for Fc heterodimerization is generally referred to as “knobs and holes” or “knobs-into-holes”. These terms refer to amino acid mutations that create steric influences to favor formation of Fc heterodimers over Fc homodimers, as described in, e.g., Ridgway et al., 1996, Protein Engineering 9(7):617; Atwell et al., 1997, J. Mol. Biol. 270:26; U.S. Pat. No. 8,216,805. Knob-in-hole mutations can be combined with other strategies to improve heterodimerization.

In one aspect, the one or more modifications to a first polypeptide of the CD19 binding molecule comprising a heavy chain constant domain can create a “knob” and the one or more modifications to a second polypeptide of the CD19 binding molecule creates a “hole,” such that heterodimerization of the polypeptide of the CD19 binding molecule comprising a heavy chain constant domain causes the “knob” to interface (e.g., interact, e.g., a CH2 domain of a first polypeptide interacting with a CH2 domain of a second polypeptide, or a CH3 domain of a first polypeptide interacting with a CH3 domain of a second polypeptide) with the “hole.” The knob projects from the interface of a first polypeptide of the CD19 binding molecule comprising a heavy chain constant domain and is therefore positionable in a compensatory “hole” in the interface with a second polypeptide of the CD19 binding molecule comprising a heavy chain constant domain so as to stabilize the heteromultimer, and thereby favor heteromultimer formation over homomultimer formation, for example. The knob can exist in the original interface or can be introduced synthetically (e.g. by altering nucleic acid encoding the interface). The import residues for the formation of a knob are generally naturally occurring amino acid residues and can be selected from arginine (R), phenylalanine (F), tyrosine (Y) and tryptophan (W). In some cases, tryptophan and tyrosine are selected. In an embodiment, the original residue for the formation of the protuberance has a small side chain volume, such as alanine, asparagine, aspartic acid, glycine, serine, threonine or valine.

A “hole” comprises at least one amino acid side chain which is recessed from the interface of a second polypeptide of the CD19 binding molecule comprising a heavy chain constant domain and therefore accommodates a corresponding knob on the adjacent interfacing surface of a first polypeptide of the CD19 binding molecule comprising a heavy chain constant domain. The hole can exist in the original interface or can be introduced synthetically (e.g. by altering nucleic acid encoding the interface). The import residues for the formation of a hole are usually naturally occurring amino acid residues and are in some embodiments selected from alanine (A), serine (S), threonine (T) and valine (V). In one embodiment, the amino acid residue is serine, alanine or threonine. In another embodiment, the original residue for the formation of the hole has a large side chain volume, such as tyrosine, arginine, phenylalanine or tryptophan.

In an embodiment, a first CH3 domain is modified at residue 366, 405 or 407 to create either a “knob” or a hole” (as described above), and the second CH3 domain that heterodimerizes with the first CH3 domain is modified at: residue 407 if residue 366 is modified in the first CH3 domain, residue 394 if residue 405 is modified in the first CH3 domain, or residue 366 if residue 407 is modified in the first CH3 domain to create a “hole” or “knob” complementary to the “knob” or “hole” of the first CH3 domain.

In another embodiment, a first CH3 domain is modified at residue 366, and the second CH3 domain that heterodimerizes with the first CH3 domain is modified at residues 366, 368 and/or 407, to create a “hole” or “knob” complementary to the “knob” or “hole” of the first CH3 domain. In one embodiment, the modification to the first CH3 domain introduces a tyrosine (Y) residue at position 366. In an embodiment, the modification to the first CH3 is T366Y. In one embodiment, the modification to the first CH3 domain introduces a tryptophan (W) residue at position 366. In an embodiment, the modification to the first CH3 is T366W. In some embodiments, the modification to the second CH3 domain that heterodimerizes with the first CH3 domain modified at position 366 (e.g., has a tyrosine (Y) or tryptophan (W) introduced at position 366, e.g., comprises the modification T366Y or T366W), comprises a modification at position 366, a modification at position 368 and a modification at position 407. In some embodiments, the modification at position 366 introduces a serine (S) residue, the modification at position 368 introduces an alanine (A), and the modification at position 407 introduces a valine (V). In some embodiments, the modifications comprise T366S, L368A and Y407V. In one embodiment, the first CH3 domain of the multispecific molecule comprises the modification T366Y, and the second CH3 domain that heterodimerizes with the first CH3 domain comprises the modifications T366S, L368A and Y407V, or vice versa. In one embodiment, the first CH3 domain of the multispecific molecule comprises the modification T366W, and the second CH3 domain that heterodimerizes with the first CH3 domain comprises the modifications T366S, L368A and Y407V, or vice versa.

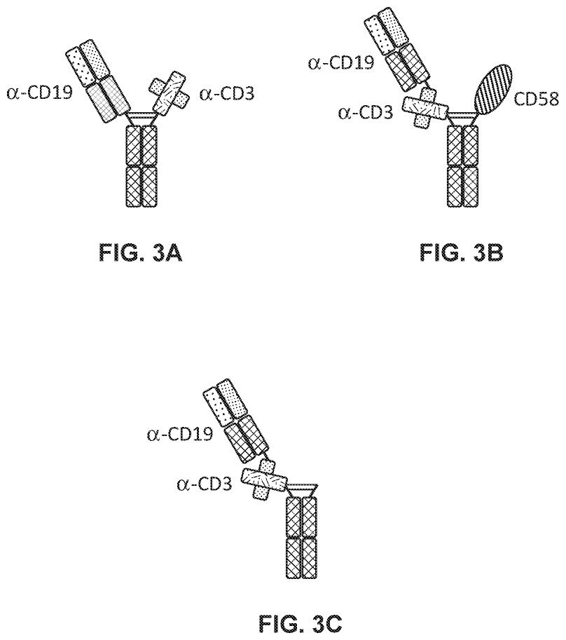

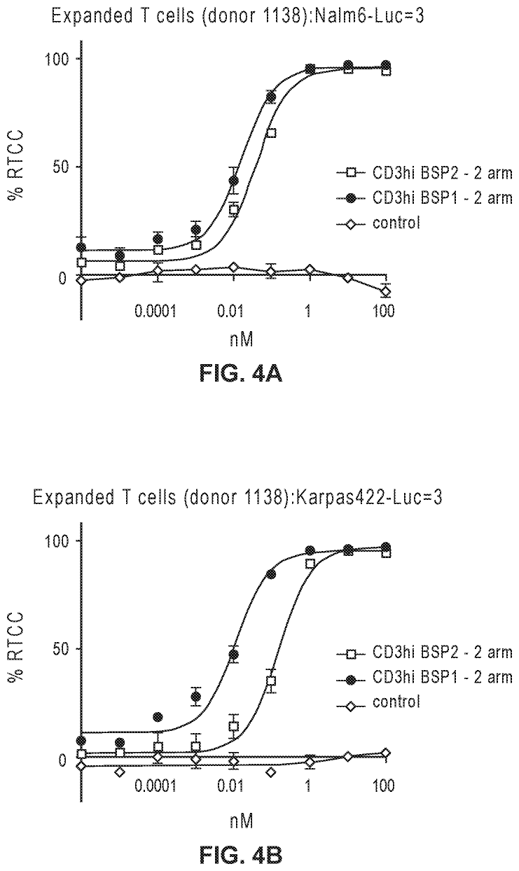

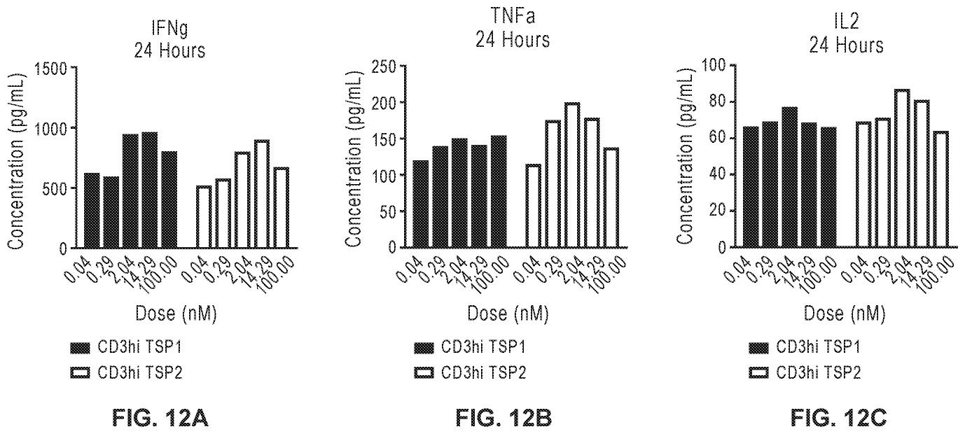

Additional steric or “skew” (e.g., knob in hole) modifications are described in PCT publication no. WO2014/145806 (for example, FIG. 3, FIG. 4 and FIG. 12 of WO2014/145806), PCT publication no. WO2014/110601, and PCT publication no. WO 2016/086186, WO 2016/086189, WO 2016/086196 and WO 2016/182751. An example of a KIH variant comprises a first constant chain comprising a L368D and a K370S modification, paired with a second constant chain comprising a S364K and E357Q modification.

Additional knob in hole modification pairs suitable for use in any of the CD19 binding molecules of the present disclosure are further described in, for example, WO1996/027011, and Merchant et al., 1998, Nat. Biotechnol., 16:677-681.

In further embodiments, the CH3 domains can be additionally modified to introduce a pair of cysteine residues. Without being bound by theory, it is believed that the introduction of a pair of cysteine residues capable of forming a disulfide bond provide stability to heterodimerized CD19 binding molecules, e.g., MBMs, comprising paired CH3 domains. In some embodiments, the first CH3 domain comprises a cysteine at position 354, and the second CH3 domain that heterodimerizes with the first CH3 domain comprises a cysteine at position 349. In some embodiments, the first CH3 domain comprises a cysteine at position 354 (e.g., comprises the modification S354C) and a tyrosine (Y) at position 366 (e.g., comprises the modification T366Y), and the second CH3 domain that heterodimerizes with the first CH3 domain comprises a cysteine at position 349 (e.g., comprises the modification Y349C), a serine at position 366 (e.g., comprises the modification T366S), an alanine at position 368 (e.g., comprises the modification L368A), and a valine at position 407 (e.g., comprises the modification Y407V). In some embodiments, the first CH3 domain comprises a cysteine at position 354 (e.g., comprises the modification S354C) and a tryptophan (W) at position 366 (e.g., comprises the modification T366WV), and the second CH3 domain that heterodimerizes with the first CH3 domain comprises a cysteine at position 349 (e.g., comprises the modification Y349C), a serine at position 366 (e.g., comprises the modification T366S), an alanine at position 368 (e.g., comprises the modification L368A), and a valine at position 407 (e.g., comprises the modification Y407V).

An additional mechanism that finds use in the generation of heterodimers is sometimes referred to as “electrostatic steering” as described in Gunasekaran et al., 2010, J. Biol. Chem. 285(25):19637. This is sometimes referred to herein as “charge pairs”. In this embodiment, electrostatics are used to skew the formation towards heterodimerization. As a skilled artisan will appreciate, these can also have an effect on pl, and thus on purification, and thus could in some cases also be considered pl variants. However, as these were generated to force heterodimerization and were not used as purification tools, they are classified as “steric variants”. These include, but are not limited to, D221 E/P228E/L368E paired with D221R/P228R/K409R and C220E/P228E/368E paired with C220R/E224R/P228R/K409R.

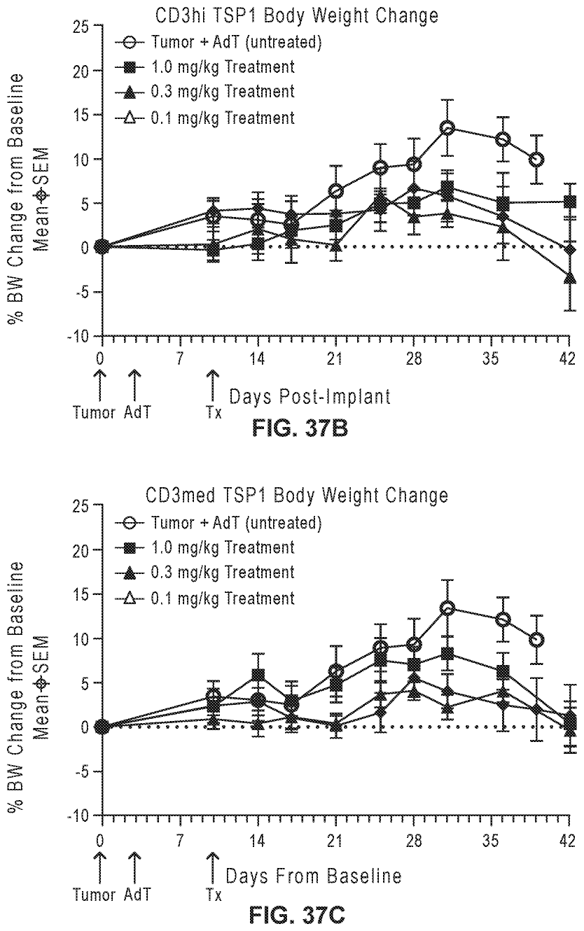

Additional variants that can be combined with other variants, optionally and independently in any amount, such as pl variants outlined herein or other steric variants that are shown in FIG. 37 of US 2012/0149876.

In some embodiments, the steric variants outlined herein can be optionally and independently incorporated with any pl variant (or other variants such as Fc variants, FcRn variants) into one or both Fc regions, and can be independently and optionally included or excluded from the CD19 binding molecules of the disclosure.

A list of suitable skew variants is found in Table 5 showing some pairs of particular utility in many embodiments. Of particular use in many embodiments are the pairs of sets including, but not limited to, S364K/E357Q:L368D/K370S; L368D/K370S:S364K; L368E/K370S: S364K; T411T/E360E/Q362E:D401K; L368D/K370S:S364K/E357L; and K370S:S364K/E357Q. In terms of nomenclature, the pair “S364K/E357Q:L368D/K370S” means that one of the Fc regions has the double variant set S364K/E357Q and the other has the double variant set L368D/K370S.

TABLE 5
Exemplary skew variants

| Fc region 1 | Fc region 2 |
|---|---|
| F405A | T394F |
| S364D | Y349K |
| S364E | L368K |
| S364E | Y349K |
| S364F | K370G |
| S364H | Y349K |
| S364H | Y349T |
| S364Y | K370G |
| T411K | K370E |
| V397S/F405A | T394F |
| K370R/T411K | K370E/T411E |
| L351E/S364D | Y349K/L351K |
| L351E/S364E | Y349K/L351K |
| L351E/T366D | L351K/T366K |
| P395T/V397S/F405A | T394F |
| S364D/K370G | S364Y/K370R |
| S364D/T394F | Y349K/F405A |
| S364E/F405A | Y349K/T394F |
| S364E/F405S | Y349K/T394Y |
| S364E/T411E | Y349K/D401K |
| S364H/D401K | Y349T/T411E |
| S364H/F405A | Y349T/T394F |
| S364H/T394F | Y349T/F405A |
| Y349C/S364E | Y349K/S354C |
| L351E/S364D/F405A | Y349K/L351K/T394F |
| L351K/S364H/D401K | Y349T/L351E/T411E |
| S364E/T411E/F405A | Y349K/T394F/D401K |
| S364H/D401K/F405A | Y349T/T394F/T411E |
| S364H/F405A/T411E | Y349T/T394F/D401K |
| K370E/T411D | T411K |
| L368E/K409E | L368K |
| Y349T/T394F/S354C | S364H/F405A/Y349C |
| T411E | D401K |
| T411E | D401R/T411R |
| Q347E/K360E | Q347R |
| L368E | S364K |
| L368E/K370S | S364K |
| L368E/K370T | S364K |
| L368E/D401R | S364K |
| L368E/D401N | S364K |
| L368E | E357S/S364K |
| L368E | S364K/K409E |
| L368E | S364K/K409V |
| L368D | S364K |
| L368D/K370S | S364K |
| L368D/K370S | S364K/E357L |
| L368D/K370S | S364K/E357Q |
| T411E/K360E/Q362E | D401K |
| K370S | S364K |
| L368E/K370S | S364K/E357Q |
| K370S | S364K/E357Q |
| T411E/K360D | D401K |
| T411E/K360E | D401K |
| T411E/Q362E | D401K |
| T411E/N390D | D401K |
| T411E | D401K/Q347K |
| T411E | D401K/Q347R |
| T411E/K360D/Q362E | D401K |
| K392D/K409D | E356K/D399K |
| K370D/K392D/K409D | E356K/E357K/D399K |
| I199T/N203D/K247Q/R355Q/N384S/ | Q196K/I199T/P217R/ |
| K392N/V397M/Q419E/K447_{—} | P228R/N276K |
| I199T/N203D/K247Q/R355Q/N384S/ | Q196K/I199T/N276K |
| K392N/V397M/Q419E/K447_{—} | |
| N384S/K392N/V397M/Q419E | N276K |
| D221E/P228E/L368E | D221R/P228R/K409R |
| C220E/P228E/L368E | C220R/E224R/P228R/ |
| | K409R |
| F405L | K409R |
| T366I/K392M/T394W | F405A/Y407V |
| T366V/K409F | L351Y/Y407A |
| T366A/K392E/K409F/T411E | D399R/S400R/Y407A |
| L351K | L351E |
| I199T/N203D/K247Q/R355Q/Q419E/K447_{—} | Q196K/I199T/P217R/ |
| | P228R/N276K |
| I199T/N203D/K247Q/R355Q/Q419E/K447_{—} | Q196K/I199T/N276K |
| I199T N203D K274Q R355Q N384S K392N | |
| V397M Q419E DEL447 | |
| N208D Q295E N384D Q418E N421D | |
| N208D Q295E Q418E N421D | |
| Q196K I199T P217R P228R N276K | |
| Q196K I199T N276K | |
| E269Q E272Q E283Q E357Q | |
| E269Q E272Q E283Q | |
| E269Q E272Q | |
| E269Q E283Q | |
| E272Q E283Q | |
| E269Q | |
| T411E/K360E/N390D | D401K |
| T411E/Q362E/N390D | D401K |
| T411E/Q347R | D401K/K360D |
| T411E/Q347R | D401K/K360E |
| T411E/K360 | D401K/Q347K |
| T411E/K360D | D401K/Q347R |
| T411E/K360E | D401K/Q347K |
| T411E/K360E | D401K/Q347R |
| T411E/S364K | D401K/K370S |
| T411E/K370S | D401K/S364K |
| Q347E | E357Q |
| Q347E | E357Q/Q362K |
| K360D/Q362E | Q347R |
| K360D/Q362E | D401K |
| K360D/Q362E | Q347R/D401K |
| K360E/Q362E | Q347R |
| K360E/Q362E | D401K |
| K360E/Q362E | Q347R/D401K |
| Q362E/N390D | D401K |
| Q347E/K360D | D401N |
| K360D | Q347R/N390K |
| K360D | N390K/D401N |
| K360E | Y349H |
| K370S/Q347E | S364K |
| K370S/E357L | S364K |
| K370S/E357Q | S364K |
| K370S/Q347E/E357L | S364K |
| K370S/Q347E/E357Q | S364K |
| L368D/K370S/Q347E | S364K |
| L368D/K370S/E357L | S364K |
| L368D/K370S/E357Q | S364K |
| L368D/K370S/Q347E/E357L | S364K |
| L368D/K370S/Q347E/E357Q | S364K |
| L368E/K370S/Q347E | S364K |
| L368E/K370S/E357L | S364K |
| L368E/K370S/E357Q | S364K |
| L368E/K370S/Q347E/E357L | S364K |
| L368E/K370S/Q347E/E357Q | S364K |
| L368D/K370T/Q347E | S364K |
| L368D/K370T/E357L | S364K |
| L368D/K370T/E357Q | S364K |
| L368D/K370T/Q347E/E357L | S364K |
| L368D/K370T/Q347E/E357Q | S364K |
| L368E/K370T/Q347E | S364K |
| L368E/K370T/E357L | S364K |
| L368E/K370T/E357Q | S364K |
| L368E/K370T/Q347E/E357L | S364K |
| L368E/K370T/Q347E/E357Q | S364K |
| T411E/Q362E | D401K/T411K |
| T411E/N390D | D401K/T411K |
| T411E/Q362E | D401R/T411R |
| T411E/N390D | D401R/T411R |
| Y407T | T366Y |
| F405A | T394W |
| T366Y/F405A | T394W/Y407T |
| Y407A | T366W |
| T366S/L368A/Y407V | T366W |
| T366S/L368A/Y407V/Y349C | T366W/S354C |
| K392D/K409D | E356K/D399K |
| K370D/K392D/K409D | E356K/E357K/D399K |
| I199T/N203D/K247Q/R355Q/N384S/ | Q196K/I199T/P217R/ |
| K392N/V397M/Q419E/K447_{—} | P228R/N276K |
| I199T/N203D/K247Q/R355Q/N384S/ | Q196K/I199T/N276K |
| K392N/V397M/Q419E/K447_{—} | |
| N384S/K392N/V397M/Q419E | N276K |
| D221E/P228E/L368E | D221R/P228R/K409R |
| C220E/P228E/L368E | C220R/E224R/P228R/ |
| | K409R |
| F405L | K409R |
| T366I/K392M/T394W | F405A/Y407V |
| T366V/K409F | L351Y/Y407A |
| T366A/K392E/K409F/T411E | D399R/S400R/Y407A |
| L351K | L351E |
| I199T/N203D/K247Q/R355Q/Q419E/K447_{—} | Q196K/I199T/P217R/ |
| | P228R/N276K |
| I199T/N203D/K247Q/R355Q/Q419E/K447_{—} | Q196K/I199T/N276K |
| I199T N203D K274Q R355Q N384S K392N | |
| V397M Q419E DEL447 | |
| N208D Q295E N384D Q418E N421D | |
| Q295E N384D Q418E N421D | |
| N208D Q295E Q418E N421D | |
| Q295E Q418E N421D | |
| Q196K I199T P217R P228R N276K | |
| Q196K I199T N276K | |
| E269Q E272Q E283Q E357Q | |
| E269Q E272Q E283Q | |
| E269Q E272Q | |
| E269Q E283Q | |
| E272Q E283Q | |
| E269Q | |

In some embodiments, a CD19 binding molecule comprises a first Fc region and a second Fc region. In some embodiments, the first Fc region comprises the following mutations: L368D and K370S, and the second Fc region comprises the following mutations: S364K and E357Q. In some embodiments, the first Fc region comprises the following mutations: S364K and E357Q, and the second Fc region comprises the following mutations: L368D and K370S.

### (b) Alternative Knob and Hole: IgG Heterodimerization

Heterodimerization of polypeptide chains of a CD19 binding molecule comprising paired CH3 domains can be increased by introducing one or more modifications in a CH3 domain which is derived from the IgG1 antibody class. In an embodiment, the modifications comprise a K409R modification to one CH3 domain paired with F405L modification in the second CH3 domain. Additional modifications can also, or alternatively, be at positions 366, 368, 370, 399, 405, 407, and 409. In some cases, heterodimerization of polypeptides comprising such modifications is achieved under reducing conditions, e.g., 10-100 mM 2-MEA (e.g., 25, 50, or 100 mM 2-MEA) for 1-10, e.g., 1.5-5, e.g., 5, hours at 25-37C, e.g., 25C or 37C.

The amino acid replacements described herein can be introduced into the CH3 domains using techniques which are well known (see, e.g., McPherson, ed., 1991, Directed Mutagenesis: a Practical Approach; Adelman et al., 1983, DNA, 2:183).

The IgG heterodimerization strategy is further described in, for example, WO2008/119353, WO2011/131746, and WO2013/060867.

In any of the embodiments described in this Section, the CH3 domains can be additionally modified to introduce a pair of cysteine residues as described in Section 7.2.2.1.3.

### (c) pl (Isoelectric Point) Variants

In general, as will be appreciated by a skilled artisan, there are two general categories of pl variants: those that increase the pl of the protein (basic changes) and those that decrease the pl of the protein (acidic changes). As described herein, all combinations of these variants can be done: one Fc region can be wild type, or a variant that does not display a significantly different pl from wild-type, and the other can be either more basic or more acidic. Alternatively, each Fc region is changed, one to more basic and one to more acidic.

Exemplary combinations of pl variants are shown in Table 6. As outlined herein and shown in Table 6, these changes are shown relative to IgG1, but all isotypes can be altered this way, as well as isotype hybrids. In the case where the heavy chain constant domain is from IgG2-4, R133E and R133Q can also be used.

TABLE 6
Exemplary pI Variant Combinations

| Variant constant region | Substitutions |
|---|---|
| pI_ISO(−) | I199T N203D K274Q R355Q N384S |
| | K392N V397M Q419E DEL447 |
| pI_(−)_isosteric_A | N208D Q295E N384D Q418E |
| | N421D |
| pI_(−)_isosteric A-Fc only | Q295E N384D Q418E N421D |
| pI_(−)_isosteric_B | N208D Q295E Q418E N421D |
| pI_(−)_isosteric_B-Fc only | Q295E Q418E N421D |
| pI_ISO(+RR) | Q196K I199T P217R P228R N276K |
| pI_ISO(+) | Q196K I199T N276K |
| pI_(+)_isosteric_A | E269Q E272Q E283Q E357Q |
| pI_(+)_isosteric_B | E269Q E272Q E283Q |
| pI_(+)_isosteric_E269Q/E272Q | E269Q E272Q |
| pI_(+)_isosteric_E269Q/E283Q | E269Q E283Q |
| pI_(+)_isosteric_E272Q/E283Q | E272Q E283Q |
| pI_(+)_isosteric_E269Q | E269Q |

In one embodiment, for example in the FIG. 1B-1W, FIG. 1Y-1AH, FIG. 2B-2L, and FIG. 2N-2V formats, a combination of pl variants has one Fc region (the negative Fab side) comprising 208D/295E/384D/418E/421 D variants (N208D/Q295E/N384D/Q418E/N421 D when relative to human IgG1) and a second Fc region (the positive scFv side) comprising a positively charged scFv linker, e.g., L36 (described in Section 7.2.2.3). However, as will be appreciated by a skilled artisan, the first Fc region includes a CH1 domain, including position 208. Accordingly, in constructs that do not include a CH1 domain (for example for MBMs that do not utilize a CH1 domain as one of the domains, for example in a format depicted in FIG. 2K), a negative pl variant Fc set can include 295E/384D/418E/421D variants (Q295E/N384D/Q418E/N421D when relative to human IgG1).

In some embodiments, a first Fc region has a set of substitutions from Table 6 and a second Fc region is connected to a charged linker (e.g., selected from those described in Section 7.2.2.3).

In some embodiments, the CD19 binding molecule of the present disclosure comprises a first Fc region and a second Fc region. In some embodiments, the first Fc region comprises the following mutations: N208D, Q295E, N384D, Q418E, and N421D. In some embodiments, the second Fc region comprises the following mutations: N208D, Q295E, N384D, Q418E, and N421D.

### (d) Isotopic Variants

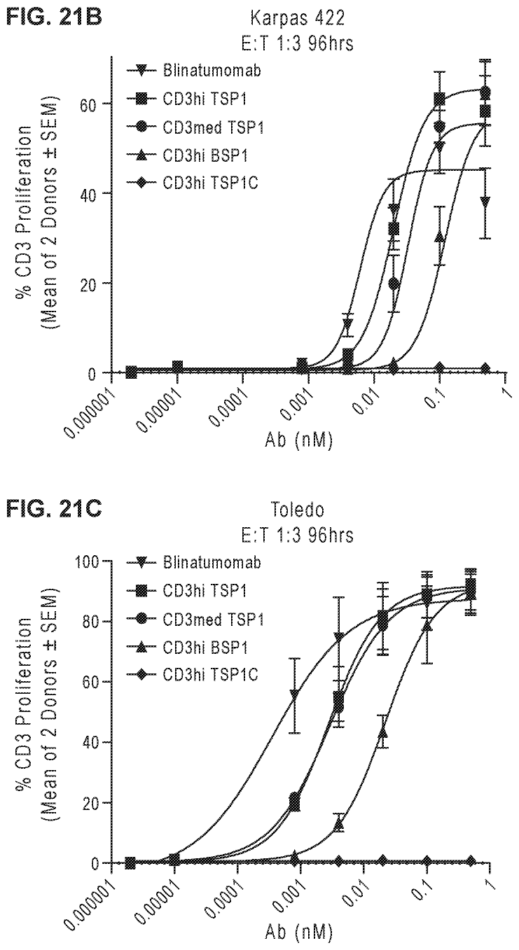

In addition, many embodiments of the disclosure rely on the “importation” of pl amino acids at particular positions from one IgG isotype into another, thus reducing or eliminating the possibility of unwanted immunogenicity being introduced into the variants. A number of these are shown in FIG. 21 of US Publ. 2014/0370013. That is, IgG1 is a common isotype for therapeutic antibodies for a variety of reasons, including high effector function. However, the heavy constant region of IgG1 has a higher pl than that of IgG2 (8.10 versus 7.31). By introducing IgG2 residues at particular positions into the IgG1 backbone, the pl of the resulting Fc region is lowered (or increased) and additionally exhibits longer serum half-life. For example, IgG1 has a glycine (pl 5.97) at position 137, and IgG2 has a glutamic acid (pl 3.22); importing the glutamic acid will affect the pl of the resulting protein. As is described below, a number of amino acid substitutions are generally required to significantly affect the pl of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pl amino acid to a lower pl amino acid), or to allow accommodations in structure for stability, as is further described below.

In addition, by pl engineering both the heavy and light constant domains of a CD19 binding molecule comprising two half antibodies, significant changes in each half antibody can be seen. Having the pls of the two half antibodies differ by at least 0.5 can allow separation by ion exchange chromatography or isoelectric focusing, or other methods sensitive to isoelectric point.

### (e) Calculating pl

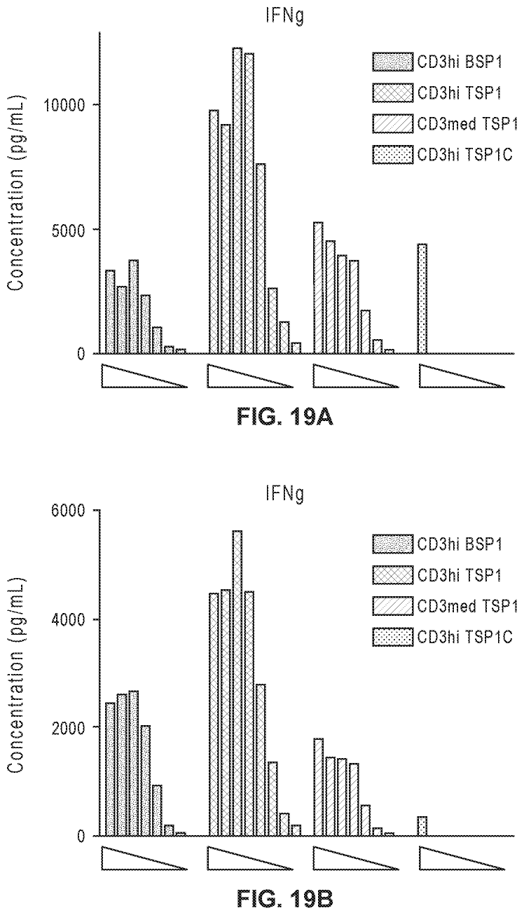

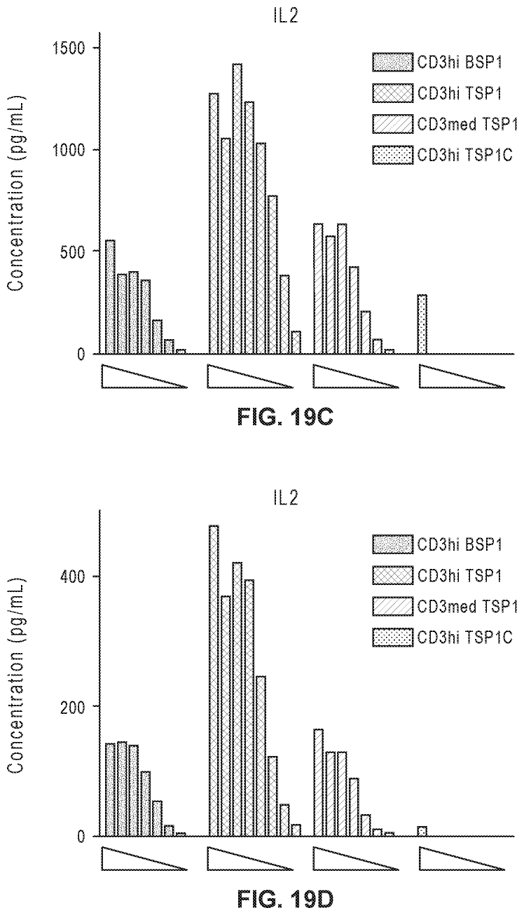

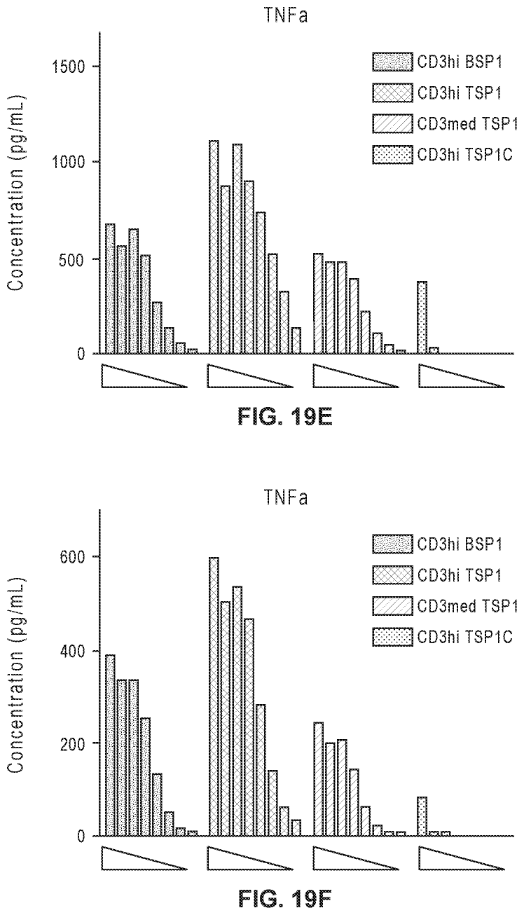

The pl of a half antibody comprising an Fc region and an ABM or ABM chain can depend on the pl of the variant heavy chain constant domain and the pl of the total half antibody, including the variant heavy chain constant domain and ABM or ABM chain. Thus, in some embodiments, the change in pl is calculated on the basis of the variant heavy chain constant domain, using the chart in the FIG. 19 of US Pub. 2014/0370013. As discussed herein, which half antibody to engineer is generally decided by the inherent pl of the half antibodies. Alternatively, the pl of each half antibody can be compared.

(f) pl Variants that Also Confer Better FcRn In Vivo Binding

In the case where a pl variant decreases the pl of an Fc region, it can have the added benefit of improving serum retention in vivo.

pl variant Fc regions are believed to provide longer half-lives to antigen binding molecules in vivo, because binding to FcRn at pH 6 in an endosome sequesters the Fc (Ghetie and Ward, 1997, Immunol Today. 18(12): 592-598). The endosomal compartment then recycles the Fc to the cell surface. Once the compartment opens to the extracellular space, the higher pH ˜7.4, induces the release of Fc back into the blood. In mice, Dall'Acqua et al. showed that Fc mutants with increased FcRn binding at pH 6 and pH 7.4 actually had reduced serum concentrations and the same half life as wild-type Fc (Dall'Acqua et al. 2002, J. Immunol. 169:5171-5180). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

It has been suggested that antibodies with variable regions that have lower isoelectric points can also have longer serum half-lives (Igawa et al., 2010, PEDS. 23(5): 385-392). However, the mechanism of this is still poorly understood. Moreover, variable regions differ from antibody to antibody. Constant region variants with reduced pl and extended half-life would provide a more modular approach to improving the pharmacokinetic properties of CD19 binding molecules, as described herein.

### (g) Polar Bridge

Heterodimerization of polypeptide chains of CD19 binding molecules, e.g., MBMs, comprising an Fc domain can be increased by introducing modifications based on the “polar-bridging” rationale, which is to make residues at the binding interface of the two polypeptide chains to interact with residues of similar (or complimentary) physical property in the heterodimer configuration, while with residues of different physical property in the homodimer configuration. In particular, these modifications are designed so that, in the heterodimer formation, polar residues interact with polar residues, while hydrophobic residues interact with hydrophobic residues. In contrast, in the homodimer formation, residues are modified so that polar residues interact with hydrophobic residues. The favorable interactions in the heterodimer configuration and the unfavorable interactions in the homodimer configuration work together to make it more likely for Fc regions to form heterodimers than to form homodimers.

In an exemplary embodiment, the above modifications are generated at one or more positions of residues 364, 368, 399, 405, 409, and 411 of a CH3 domain.

In some embodiments, one or more modifications selected from the group consisting of S364L, T366V, L368Q, N399K, F405S, K409F and R411K are introduced into one of the two CH3 domains. One or more modifications selected from the group consisting of Y407F, K409Q and T411N can be introduced into the second CH3 domain.

In another embodiment, one or more modifications selected from the group consisting of S364L, T366V, L368Q, D399K, F405S, K409F and T411K are introduced into one CH3 domain, while one or more modifications selected from the group consisting of Y407F, K409Q and T411D are introduced into the second CH3 domain.

In one exemplary embodiment, the original residue of threonine at position 366 of one CH3 domain is replaced by valine, while the original residue of tyrosine at position 407 of the other CH3 domain is replaced by phenylalanine.

In another exemplary embodiment, the original residue of serine at position 364 of one CH3 domain is replaced by leucine, while the original residue of leucine at position 368 of the same CH3 domain is replaced by glutamine.

In yet another exemplary embodiment, the original residue of phenylalanine at position 405 of one CH3 domain is replaced by serine and the original residue of lysine at position 409 of this CH3 domain is replaced by phenylalanine, while the original residue of lysine at position 409 of the other CH3 domain is replaced by glutamine.

In yet another exemplary embodiment, the original residue of aspartic acid at position 399 of one CH3 domain is replaced by lysine, and the original residue of threonine at position 411 of the same CH3 domain is replaced by lysine, while the original residue of threonine at position 411 of the other CH3 domain is replaced by aspartic acid.

The amino acid replacements described herein can be introduced into the CH3 domains using techniques which are well known (see, e.g., McPherson, ed., 1991, Directed Mutagenesis: a Practical Approach; Adelman et al., 1983, DNA, 2:183). The polar bridge strategy is described in, for example, WO2006/106905, WO2009/089004 and Gunasekaran et al., 2010, JBC 285:19637-19646.

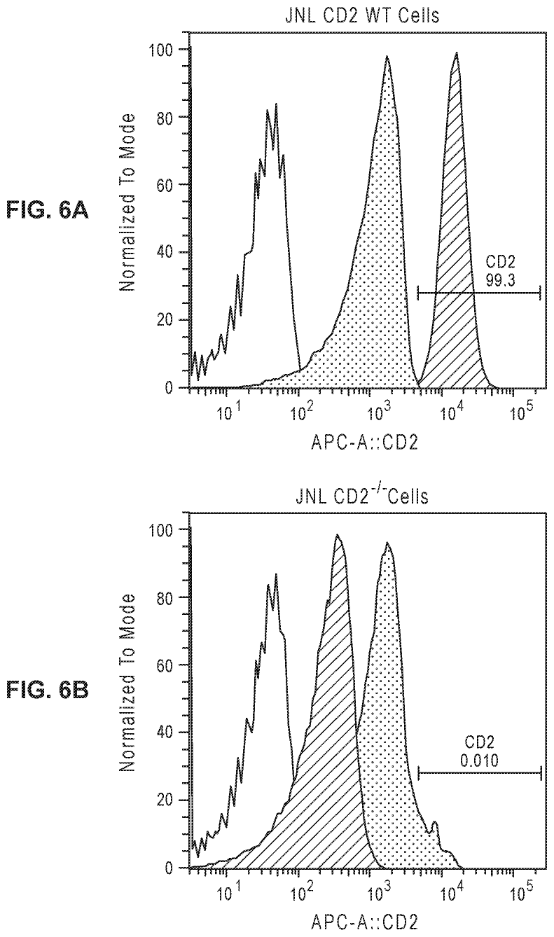

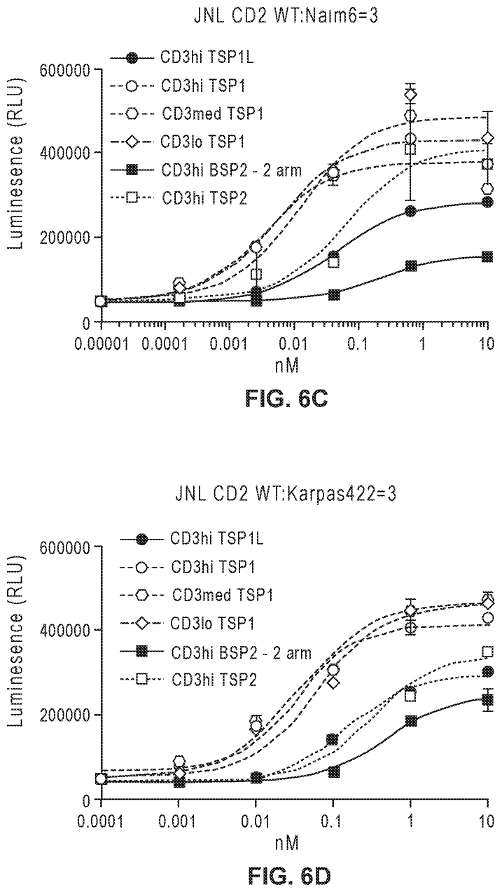

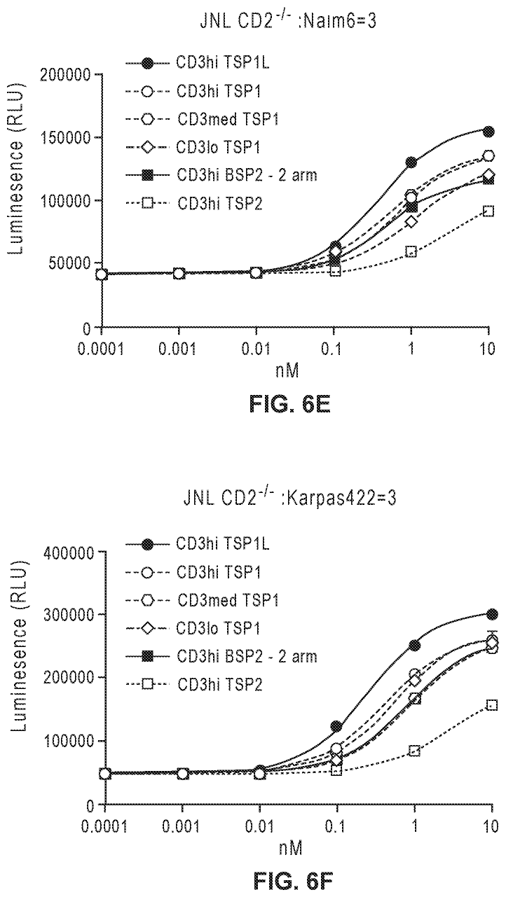

Additional polar bridge modifications are described in, for example, PCT publication no. WO2014/145806 (for example, FIG. 6 of WO2014/145806), PCT publication no. WO2014/110601, and PCT publication no. WO 2016/086186, WO 2016/086189, WO 2016/086196 and WO 2016/182751. An example of a polar bridge variant comprises a constant chain comprising a N208D, Q295E, N384D, Q418E and N421D modification.

In any of the embodiments described herein, the CH3 domains can be additionally modified to introduce a pair of cysteine residues as described in Section 7.2.2.1.3.

Additional strategies for enhancing heterodimerization are described in, for example, WO2016/105450, WO2016/086186, WO2016/086189, WO2016/086196, WO2016/141378, and WO2014/145806, and WO2014/110601. Any of the strategies can be employed in a CD19 binding molecule described herein.

### 7.2.2.1.6. Combination of Heterodimerization Variants and Other Fc Variants

As will be appreciated by a skilled artisan, all of the recited heterodimerization variants (including skew and/or pl variants) can be optionally and independently combined in any way, as long as the Fc regions of an Fc domain retain their ability to dimerize. In addition, all of these variants can be combined into any of the heterodimerization formats.

In the case of pl variants, while embodiments finding particular use are shown in Table 6, other combinations can be generated, following the basic rule of altering the pl difference between two Fc regions in an Fc heterodimer to facilitate purification.

In addition, any of the heterodimerization variants, skew and pl, are also independently and optionally combined with Fc ablation variants, Fc variants, FcRn variants, as generally outlined herein.

In some embodiments, a particular combination of skew and pl variants that finds use in the present disclosure is T366S/L368A/Y407V:T366W (optionally including a bridging disulfide, T366S/L368A/Y407V/Y349C:T366W/S354C) with one Fc region comprising Q295E/N384D/Q418E/N481 D and the other a positively charged scFv linker (when the format includes an scFv domain). As will be appreciated by a skilled artisan, the “knobs in holes” variants do not change pl, and thus can be used on either one of the Fc regions in an Fc heterodimer.

In some embodiments, first and second Fc regions that find use the present disclosure include the amino acid substitutions S364K/E357Q:L368D/K370S, where the first and/or second Fc region includes the ablation variant substitutions 233P/L234V/L235A/G236del/S267K, and the first and/or second Fc region comprises the pl variant substitutions N208D/Q295E/N384D/Q418E/N421D (pl_(−)_isosteric_A).

### 7.2.2.2. Hinge Regions

The CD19 binding molecules can also comprise hinge regions, e.g., connecting an antigen-binding domain to an Fc region. The hinge region can be a native or a modified hinge region. Hinge regions are typically found at the N-termini of Fc regions.

A native hinge region is the hinge region that would normally be found between Fab and Fc domains in a naturally occurring antibody. A modified hinge region is any hinge that differs in length and/or composition from the native hinge region. Such hinges can include hinge regions from other species, such as human, mouse, rat, rabbit, shark, pig, hamster, camel, llama or goat hinge regions. Other modified hinge regions can comprise a complete hinge region derived from an antibody of a different class or subclass from that of the heavy chain Fc region. Alternatively, the modified hinge region can comprise part of a natural hinge or a repeating unit in which each unit in the repeat is derived from a natural hinge region. In a further alternative, the natural hinge region can be altered by converting one or more cysteine or other residues into neutral residues, such as serine or alanine, or by converting suitably placed residues into cysteine residues. By such means the number of cysteine residues in the hinge region can be increased or decreased. This approach is described further in U.S. Pat. No. 5,677,425 by Bodmer et al., Altering the number of cysteine residues in a hinge region can, for example, facilitate assembly of light and heavy chains, or increase or decrease the stability of a 0019 binding molecule. Other modified hinge regions can be entirely synthetic and can be designed to possess desired properties such as length, cysteine composition and flexibility.

A number of modified hinge regions have been described for example, in U.S. Pat. No. 5,677,425, WO9915549, WO2005003170, WO2005003169, WO2005003170, WO9825971 and WO2005003171.

Examples of suitable hinge sequences are shown in Table 7.

TABLE 7
Hinge Sequences

| Hinge | Hinge | | SEQ ID |
|---|---|---|---|
| Name | Description | Hinge Sequence | NO: |
| H1 | Human IgA1 | VPSTPPTPSPSTPPTPSPS | 1181 |
| H2 | Human IgA2 | VPPPPP | 1182 |
| H3 | Human IgD | ESPKAQASSVPTAQPQAEGSLAKATTAPATTRN | 1183 |
| | | TGRGGEEKKKEKEKEEQEERETKTP | |
| H4 | Human IgG1 | EPKSCDKTHTCPPCP | 1184 |
| H5 | Human IgG2 | ERKCCVECPPCP | 1185 |
| H6 | Human IgG3 | ELKTPLGDTTHTCPRCPEPKSCDTPPPCPRCPE | 1186 |
| | | PKSCDTPPPCPRCPEPKSCDTPPPCPRCP | |
| H7 | Human IgG4 | ESKYGPPCPSCP | 1187 |
| H8 | Human IgG4(P) | ESKYGPPCPPCP | 1188 |
| H9 | Engineered v1 | CPPC | 1179 |
| H10 | Engineered v2 | CPSC | 1189 |
| H11 | Engineered v3 | CPRC | 1190 |
| H12 | Engineered v4 | SPPC | 1191 |
| H13 | Engineered v5 | CPPS | 1192 |
| H14 | Engineered v6 | SPPS | 1180 |
| H15 | Engineered v7 | DKTHTCAA | 1193 |
| H16 | Engineered v8 | DKTHTCPPCPA | 1194 |
| H17 | Engineered v9 | DKTHTCPPCPATCPPCPA | 1195 |
| H18 | Engineered v10 | DKTHTCPPCPATCPPCPATCPPCPA | 1196 |
| H19 | Engineered v11 | DKTHTCPPCPAGKPTLYNSLVMSDTAGTCY | 1197 |
| H20 | Engineered v12 | DKTHTCPPCPAGKPTHVNVSVVMAEVDGTCY | 1198 |
| H21 | Engineered v13 | DKTHTCCVECPPCPA | 1199 |
| H22 | Engineered v14 | DKTHTCPRCPEPKSCDTPPPCPRCPA | 1200 |
| H23 | Engineered v15 | DKTHTCPSCPA | 1201 |

In one embodiment, the heavy chain Fc region possesses an intact hinge region at its N-terminus.

In one embodiment, the heavy chain Fc region and hinge region are derived from IgG4 and the hinge region comprises the modified sequence CPPC (SEQ ID NO: 1179). The core hinge region of human IgG4 contains the sequence CPSC (SEQ ID NO: 1189) compared to IgG1 which contains the sequence CPPC (SEQ ID NO: 1179). The serine residue present in the IgG4 sequence leads to increased flexibility in this region, and therefore a proportion of molecules form disulfide bonds within the same protein chain (an intrachain disulfide) rather than bridging to the other heavy chain in the IgG molecule to form the interchain disulfide. (Angel et al., 1993, Mol Immunol 30(1):105-108). Changing the serine residue to a proline to give the same core sequence as IgG1 allows complete formation of inter-chain disulfides in the IgG4 hinge region, thus reducing heterogeneity in the purified product. This altered isotype is termed IgG4P.

### 7.2.2.3. ABM Linkers

In certain aspects, the present disclosure provides CD19 binding molecules where two or more components of an ABM (e.g., a VH and a VL of an scFv), two or more ABMs, or an ABM and a non-ABM domain (e.g., a dimerization domain such as an Fc region) are connected to one another by a peptide linker. Such linkers are referred to herein an “ABM linkers.”

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids or from 12 to 20 amino acids. In particular embodiments, a peptide linker is 2 amino acids, 3 amino acids, 4 amino acid, 5 amino acids, 6 amino acids, 7 amino acids, 8 amino acids, 9 amino acids, 10 amino acids, 11 amino acids, 12 amino acids, 13 amino acids, 14 amino acid, 15 amino acids, 16 amino acids, 17 amino acids, 18 amino acids, 19 amino acids, 20 amino acids, 21 amino acids, 22 amino acids, 23 amino acids, 24 amino acid, 25 amino acids, 26 amino acids, 27 amino acids, 28 amino acids, 29 amino acids, 30 amino acids, 31 amino acids, 32 amino acids, 33 amino acids, 34 amino acid, 35 amino acids, 36 amino acids, 37 amino acids, 38 amino acids, 39 amino acids, 40 amino acids, 41 amino acids, 42 amino acids, 43 amino acids, 44 amino acid, 45 amino acids, 46 amino acids, 47 amino acids, 48 amino acids, 49 amino acids, or 50 amino acids in length.

Charged and/or flexible linkers can be used.

Examples of flexible ABM linkers that can be used in the CD19 binding molecules include those disclosed by Chen et al., 2013, Adv Drug Deliv Rev. 65(10):1357-1369 and Klein et al., 2014, Protein Engineering, Design & Selection 27(10):325-330. A particularly useful flexible linker is (GGGGS)n (also referred to as (G4S)n) (SEQ ID NO: 1171). In some embodiments, n is any number between 1 and 10, i.e., 1, 2, 3, 4, 5, 6, 7, 8, 9, and 10, or any range bounded by any two of the foregoing numbers, e.g., 1 to 5, 2 to 5, 3 to 6, 2 to 4, 1 to 4, and so on and so forth.

Other examples of suitable ABM linkers for use in the CD19 binding molecules of the present disclosure are shown in Table 8 below:

TABLE 8
ABM Linker Sequences

| | | SEQ ID |
|---|---|---|
| Linker Name | Linker Sequence | NO: |
| L1 | ADAAP | 1202 |
| L2 | ADAAPTVSIFP | 1203 |
| L3 | ADAAPTVSIFPP | 1204 |
| L4 | AKTTAP | 1205 |
| L5 | AKTTAPSVYPLAP | 1206 |
| L6 | AKTTPKLEEGEFSEARV | 1207 |
| L7 | AKTTPKLGG | 1208 |
| L8 | AKTTPP | 1209 |
| L9 | AKTTPPSVTPLAP | 1210 |
| L10 | ASTKGP | 1211 |
| L11 | ASTKGPSVFPLAP | 1212 |
| L12 | ASTKGPSVFPLAPASTKGPSVFPLAP | 1213 |
| L13 | EGKSSGSGSESKST | 1214 |
| L14 | GEGESGEGESGEGES | 1215 |
| L15 | GEGESGEGESGEGESGEGES | 1217 |
| L16 | GEGGSGEGGSGEGGS | 1218 |
| L17 | GENKVEYAPALMALS | 1219 |
| L18 | GGEGSGGEGSGGEGS | 1220 |
| L19 | GGGESGGEGSGEGGS | 1222 |
| L20 | GGGESGGGESGGGES | 1223 |
| L21 | (GGGGS)ₙ (also referred to as (G4S)ₙ), | 1224 |
| | where n can be 1-10. | |
| L22 | GGGGSGGGGS | 1168 |
| L23 | GGGGSGGGGSGGGGS | 1174 |
| L24 | GGGGSGGGGSGGGGSGGGGS | 1173 |
| L25 | GGGKSGGGKSGGGKS | 1227 |
| L26 | GGGKSGGKGSGKGGS | 1228 |
| L27 | GGKGSGGKGSGGKGS | 1229 |
| L28 | GGSGG | 1230 |
| L29 | GGSGGGGSG | 1231 |
| L30 | GGSGGGGSGGGGS | 1232 |
| L31 | GHEAAAVMQVQYPAS | 108 |
| L32 | GKGGSGKGGSGKGGS | 109 |
| L33 | GKGKSGKGKSGKGKS | 110 |
| L34 | GKGKSGKGKSGKGKSGKGKS | 111 |
| L35 | GKPGSGKPGSGKPGS | 112 |
| L36 | GKPGSGKPGSGKPGSGKPGS | 1233 |
| L37 | GPAKELTPLKEAKVS | 1234 |
| L38 | GSAGSAAGSGEF | 1235 |
| L39 | IRPRAIGGSKPRVA | 1236 |
| L40 | KESGSVSSEQLAQFRSLD | 1237 |
| L41 | KTTPKLEEGEFSEAR | 1238 |
| L42 | QPKAAP | 1239 |
| L43 | QPKAAPSVTLFPP | 1240 |
| L44 | RADAAAA(G4S)₄ | 1241 |
| L45 | RADAAAAGGPGS | 1242 |
| L46 | RADAAP | 1243 |
| L47 | RADAAPTVS | 1244 |
| L48 | SAKTTP | 1245 |
| L49 | SAKTTPKLEEGEFSEARV | 1246 |
| L50 | SAKTTPKLGG | 1247 |
| L51 | STAGDTHLGGEDFD | 1248 |
| L52 | TVAAP | 1249 |
| L53 | TVAAPSVFIFPP | 1250 |
| L54 | TVAAPSVFIFPPTVAAPSVFIFPP | 1251 |
| L55 | GSTSGSGKPGSGEGSTKG | 1178 |
| L56 | PRGASKSGSASQTGSAPGS | 1252 |
| L57 | GTAAAGAGAAGGAAAGAAG | 134 |
| L58 | GTSGSSGSGSGGSGSGGGG | 135 |

In various aspects, the disclosure provides a CD19 binding molecule which comprises one or more ABM linkers. Each of the ABM linkers can be range from 2 amino acids to 60 amino acids in length, e.g., 4 to 30 amino acids, from 5 to 25 amino acids, from 10 to 25 amino acids or from 12 to 20 amino acids in length, optionally selected from Table 8 above. In particular embodiments, the CD19 binding molecule comprises two, three, four, five or six ABM linkers. The ABM linkers can be on one, two, three, four or even more polypeptide chains of the CD19 binding molecule.

### 7.2.3. Bispecific Binding Molecule Configurations

Exemplary BBM configurations are shown in FIG. 1. FIG. 1A shows the components of the BBM configurations shown in FIGS. 1B1-1AH. The scFv, Fab, scFab, non-immunoglobulin based ABM, and Fc domains each can have the characteristics described for these components in Sections 7.2.1 and 7.2.2. The components of the BBM configurations shown in FIG. 1 can be associated with each other by any of the means described in Sections 7.2.1 and 7.2.2 (e.g., by direct bonds, ABM linkers, disulfide bonds, Fc domains with modified with knob in hole interactions, etc.). The orientations and associations of the various components shown in FIG. 1 are merely exemplary; as will be appreciated by a skilled artisan, other orientations and associations can be suitable (e.g., as described in Sections 7.2.1 and 7.2.2).

BBMs are not limited to the configurations shown in FIG. 1. Other configurations that can be used are known to those skilled in the art. See, e.g., WO 2014/145806; WO 2017/124002; Liu et al., 2017, Front Immunol. 8:38; Brinkmann & Kontermann, 2017, mAbs 9:2, 182-212; US 2016/0355600; Klein et al., 2016, MAbs 8(6):1010-20; and US 2017/0145116.

### 7.2.3.1. Exemplary Bivalent BBMs

The BBMs can be bivalent, i.e., they have two antigen-binding domains, one of which binds CD19 (ABM1) and one of which binds a second target antigen (ABM2), e.g., a component of a TCR complex.

Exemplary bivalent BBM configurations are shown in FIGS. 1B-1F.

As depicted in FIGS. 1B-1D, a BBM can comprise two half antibodies, one comprising one ABM and the other comprising one ABM, the two halves paired through an Fc domain.

In the embodiment of FIG. 1B, the first (or left) half antibody comprises a Fab and an Fc region, and the second (or right) half antibody comprises a Fab and an Fc region. The first and second half antibodies are associated through the Fc regions forming an Fc domain.

In the embodiment of FIG. 1C, the first (or left) half antibody comprises a Fab and an Fc region, and the second (or right) half antibody comprises a scFv and an Fc region. The first and second half antibodies are associated through the Fc regions forming an Fc domain.

In the embodiment of FIG. 1D, the first (or left) half antibody comprises an scFv and an Fc region, and the second (or right) half antibody comprises an scFv and an Fc region. The first and second half antibodies are associated through the Fc regions forming an Fc domain.

As depicted in FIGS. 1E-1F, a bivalent BBM can comprise two ABMs attached to one Fc region of an Fc domain.

In the embodiment of FIG. 1E, the BBM comprises a Fab, a scFv and an Fc domain, where the scFv is located between the Fab and the Fc domain.

In the embodiment of FIG. 1F, (the “one-arm scFv-mAb” configuration) BBM comprises a Fab, a scFv and an Fc domain, where the Fab is located between the scFv and the Fc domain.

In the configuration shown in FIGS. 1B-1F, each of X and Y represent either ABM1 or ABM2, provided that the BBM comprises one ABM1 and one ABM2. Accordingly, the present disclosure provides a bivalent BBM as shown in any one of FIGS. 1B through 1F, where X is an ABM1 and Y is an ABM2 (this configuration of ABMs designated as “1” for convenience). The present disclosure also provides a bivalent BBM as shown in any one of FIGS. 1B through 1F, where X is an ABM2 and Y is an ABM1 (this configuration of ABMs designated as “B2” for convenience).

### 7.2.3.2. Exemplary Trivalent BBMs

The BBMs can be trivalent, i.e., they have three antigen-binding domains, one or two of which binds CD19 (ABM1) and one or two of which binds a second target antigen (ABM2), e.g., a component of a TCR complex.

Exemplary trivalent BBM configurations are shown in FIGS. 1G-1Z.

As depicted in FIGS. 1G-1N, 1Q-1W, 1Y-1Z a BBM can comprise two half antibodies, one comprising two ABMs and the other comprising one ABM, the two halves paired through an Fc domain.

In the embodiment of FIG. 1G, the first (or left) half antibody comprises Fab and an Fc region, and the second (or right) half antibody comprises a scFv, a Fab, and an Fc region. The first and second half antibodies are associated through the Fc regions forming an Fc domain.

In the embodiment of FIG. 1H, the first (or left) half antibody comprises a Fab and an Fc region, and the second (or right) half antibody comprises a Fab, an scFv, and an Fc region. The first and second half antibodies are associated through the Fc regions forming an Fc domain.

In the embodiment of FIG. 1I, the first (or left) half antibody comprises an scFv and an Fc region, and the second (or right) half antibody comprises two Fabs and an Fc region. The first and second half antibodies are associated through the Fc regions forming an Fc domain.

In the embodiment of FIG. 1J, the first (or left) half antibody comprises two Fav and an Fc region, and the second (or right) half antibody comprises a Fab and an Fc region. The first and second half antibodies are associated through the Fc regions forming an Fc domain.

In the embodiment of FIG. 1K, the first (or left) half antibody comprises an scFv and an Fc region, and the second (or right) half antibody comprises two scFvs and an Fc region. The first and second half antibodies are associated through the Fc regions forming an Fc domain.

In the embodiment of FIG. 1L, the first (or left) half antibody comprises an scFv and an Fc region, and the second (or right) half antibody comprises an scFv, a Fab, and an Fc region. The first and second half antibodies are associated through the Fc regions forming an Fc domain.

In the embodiment of FIG. 1M, the first (or left) half antibody comprises a scFv and an Fc region, and the second (or right) half antibody comprises a Fab, a scFv and an Fc region. The first and second half antibodies are associated through the Fc regions forming an Fc domain.

In the embodiment of FIG. 1N, the first (or left) half antibody comprises a diabody-type binding domain and an Fc region, and the second (or right) half antibody comprises a Fab and an Fc region. The first and second half antibodies are associated through the Fc regions forming an Fc domain.

In the embodiment of FIG. 1Q, the first (or left) half antibody comprises a Fab and an Fc region, and the second (or right) half antibody comprises a Fab, an Fc region, and an scFv. The first and second half antibodies are associated through the Fc regions forming an Fc domain.

In the embodiment of FIG. 1R, the first (or left) half antibody comprises a scFv and an Fc region, and the second (or right) half antibody comprises a Fab, an Fc region, and an scFv. The first and second half antibodies are associated through the Fc regions forming an Fc domain.

In the embodiment of FIG. 1S, the first (or left) half antibody comprises an scFv and an Fc region, and the second (or right) half antibody comprises an scFv, an Fc region, and a second scFv. The first and second half antibodies are associated through the Fc regions forming an Fc domain.

In the embodiment of FIG. 1T, the first (or left) half antibody comprises an scFv, an Fc region, and a Fab, and the second (or right) half antibody comprises a Fab and an Fc region. The first and second half antibodies are associated through the Fc regions forming an Fc domain.

In the embodiment of FIG. 1U, the first (or left) half antibody comprises two Fab and an Fc region, and the second (or right) half antibody comprises a non-immunoglobulin based ABM and an Fc region. The first and second half antibodies are associated through the Fc regions forming an Fc domain.

In the embodiment of FIG. 1V, the first (or left) half antibody comprises a Fab, an scFv, and an Fc region, and the second (or right) half antibody comprises a non-immunoglobulin based ABM and an Fc region. The first and second half antibodies are associated through the Fc regions forming an Fc domain.

In the embodiment of FIG. 1W, the first (or left) half antibody comprises a Fab and an Fc region, and the second (or right) half antibody comprises a scFv, a non-immunoglobulin based ABM, and an Fc region. The first and second half antibodies are associated through the Fc regions forming an Fc domain.

In the embodiment of FIG. 1Y, the first (or left) half antibody comprises an scFv and an Fc region, and the second (or right) half antibody comprises a Fab, an scFv and an Fc region. The first and second half antibodies are associated through the Fc regions forming an Fc domain.

In the embodiment of FIG. 1Z, the first (or left) half antibody comprises a Fab, an Fc region, and a scFab, and the second (or right) half antibody comprises a Fab and an Fc region. The first and second half antibodies are associated through the Fc regions forming an Fc domain.

Alternatively, as depicted in FIGS. 1O and 1P, trivalent a BBM can comprise two half antibodies, each comprising one complete ABM (a Fab in FIGS. 1O and 1P) and a portion of another ABM (one a VH, the other a VL). The two half antibodies are paired through an Fc domain, whereupon the VH and the VL associate to form a complete antigen-binding Fv domain.

The BBM can be a single chain, as shown in FIG. 1X. The BBM of FIG. 1X comprises three scFv domains connected through linkers.

In the configuration shown in FIGS. 1G-1Z, each of X, Y and A represent either an ABM1 or ABM2, provided that the BBM comprises at least ABM1 and at least one ABM2. Thus, the trivalent MBMs will include one or two ABM1s and one or two ABM2s. In some embodiments, a trivalent BBM comprises two ABM1s and one ABM2. In other embodiments, a trivalent BBM of the disclosure comprises one ABM1 and two ABM2s.

Accordingly, in the present disclosure provides a trivalent BBM as shown in any one of FIGS. 1G through 1Z, where X is an ABM1, Y is an ABM1 and A is an ABM2 (this configuration of ABMs designated as “T1” for convenience).

The disclosure further provides a trivalent BBM as shown in any one of FIGS. 1G through 1Z, where X is an ABM1, Y is an ABM2 and A is an ABM1 (this configuration of ABMs designated as “T2” for convenience).

The disclosure further provides a trivalent BBM as shown in any one of FIGS. 1G through 1Z, where X is an ABM2, Y is an ABM1 and A is an ABM1 (this configuration of ABMs designated as “T3” for convenience).

The disclosure further provides a trivalent BBM as shown in any one of FIGS. 1G through 1Z, where X is an ABM1, Y is an ABM2 and A is an ABM2 (this configuration of ABMs designated as “T4” for convenience).

The disclosure further provides a trivalent BBM as shown in any one of FIGS. 1G through 1Z, where X is an ABM2, Y is an ABM1 and A is an ABM2 (this configuration of ABMs designated as “T5” for convenience).

The disclosure further provides a trivalent BBM as shown in any one of FIGS. 1G through 1Z, where X is an ABM2, Y is an ABM2 and A is an ABM1 (this configuration of ABMs designated as “T6” for convenience).

### 7.2.3.3. Exemplary Tetravalent BBMs

The BBMs can be tetravalent, i.e., they have four antigen-binding domains, one, two, or three of which binds CD19 (ABM1) and one, two, or three of which binds a second target antigen (ABM2), e.g., a component of a TCR complex.

Exemplary tetravalent BBM configurations are shown in FIGS. 1AA-1AH.

As depicted in FIGS. 1AA-1AH, a tetravalent BBM can comprise two half antibodies, each comprising two complete ABMs, the two halves paired through an Fc domain.

In the embodiment of FIG. 1AA, the first (or left) half antibody comprises a Fab, an Fc region, and an scFv, and the second (or right) half antibody comprises a Fab, an Fc region, and an scFv. The first and second half antibodies are associated through the Fc regions forming an Fc domain.

In the embodiment of FIG. 1AB, the first (or left) half antibody comprises a Fab, an scFv, and an Fc region, and the second (or right) half antibody comprises a Fab, an scFv, and an Fc region. The first and second half antibodies are associated through the Fc regions forming an Fc domain.

In the embodiment of FIG. 1AC, the first (or left) half antibody comprises an scFv, a Fab, and an Fc region, and the second (or right) half antibody comprises an scFv, a Fab, and an Fc region. The first and second half antibodies are associated through the Fc regions forming an Fc domain.

In the embodiment of FIG. 1AD, the first (or left) half antibody comprises a Fab, an Fc region, and a second Fab, and the second (or right) half antibody comprises a Fab, an Fc region, and a second Fab. The first and second half antibodies are associated through the Fc regions forming an Fc domain.

In the embodiment of FIG. 1AE, the first (or left) half antibody comprises an scFv, a second scFv, and an Fc region, and the second (or right) half antibody comprises an scFv, a second scFv, and an Fc region. The first and second half antibodies are associated through the Fc regions forming an Fc domain.

In the embodiment of FIG. 1AF, the first (or left) half antibody comprises a Fab, an scFv, and an Fc region, and the second (or right) half antibody comprises a Fab, an scFv, and an Fc region. The first and second half antibodies are associated through the Fc regions forming an Fc domain.

In the embodiment of FIG. 1AG, the first (or left) half antibody comprises a Fab, an Fc region, and an scFv, and the second (or right) half antibody comprises a scFv, an Fc region, and a Fab. The first and second half antibodies are associated through the Fc regions forming an Fc domain.

In the embodiment of FIG. 1AH, the first (or left) half antibody comprises a scFv, an Fc region, and an Fab, and the second (or right) half antibody comprises a scFv, an Fc region, and a Fab. The first and second half antibodies are associated through the Fc regions forming an Fc domain.

In the configuration shown in FIGS. 1AA-1AH, each of X, Y, A, and B represent ABM1 or ABM2, although not necessarily in that order, and provided that the BBM comprises at least one ABM1 and at least one ABM2. Thus, the tetravalent ABMs will include one, two, or three ABM1s and one, two, or ABM2s. In some embodiments, a tetravalent BBM comprises three ABM1s and one ABM2. In other embodiments, a tetravalent BBM comprises two ABM1s two ABM2s. In yet other embodiments, a tetravalent BBM comprises one ABM1 and three ABM2s.

Accordingly, in the present disclosure provides a tetravalent BBM as shown in any one of FIGS. 1AA-1AH, where X is an ABM1 and each of Y, A, and B are ABM2s (this configuration of ABMs designated as “Tv 1” for convenience).

The disclosure further provides a tetravalent BBM as shown in any one of FIGS. 1AA-1AH, where Y is an ABM1 and each of X, A, and B are ABM2s (this configuration of ABMs designated as “Tv 2” for convenience).

The disclosure further provides a tetravalent BBM as shown in any one of FIGS. 1AA-1AH, where A is an ABM1 and each of X, Y, and B are ABM2s (this configuration of ABMs designated as “Tv 3” for convenience).

The disclosure further provides a tetravalent BBM as shown in any one of FIGS. 1AA-1AH, where B is an ABM1 and each of X, Y, and A are ABM2s (this configuration of ABMs designated as “Tv 4” for convenience).

The disclosure further provides a tetravalent BBM as shown in any one of FIGS. 1AA-1AH, where X and Y are both ABM1s and both of A and B are ABM2s (this configuration of ABMs designated as “Tv 5” for convenience).

The disclosure further provides a tetravalent BBM as shown in any one of FIGS. 1AA-1AH, where X and A are both ABM1s and both of Y and B are ABM2s (this configuration of ABMs designated as “Tv 6” for convenience).

The disclosure further provides a tetravalent BBM as shown in any one of FIGS. 1AA-1AH, where X and B are both ABM1s and both of Y and A are ABM2s (this configuration of ABMs designated as “Tv 7” for convenience).

The disclosure further provides a tetravalent BBM as shown in any one of FIGS. 1AA-1AH, where Y and A are both ABM1s and both of X and B are ABM2s (this configuration of ABMs designated as “Tv 8” for convenience).

The disclosure further provides a tetravalent BBM as shown in any one of FIGS. 1AA-1AH, where Y and B are both ABM1s and both of X and A are ABM2s (this configuration of ABMs designated as “Tv 9” for convenience).

The disclosure further provides a tetravalent BBM as shown in any one of FIGS. 1AA-1AH, where A and B are both ABM1s and both of X and Y are ABM2s (this configuration of ABMs designated as “Tv 10” for convenience).

The disclosure further provides a tetravalent BBM as shown in any one of FIGS. 1AA-1AH, where each of X, Y, and A is an ABM1 and B is an ABM2 (this configuration of ABMs designated as “Tv 11” for convenience).

The disclosure further provides a tetravalent BBM as shown in any one of FIGS. 1AA-1AH, where each of X, Y, and B is an ABM1 and A is an ABM2 (this configuration of ABMs designated as “Tv 12” for convenience).

The disclosure further provides a tetravalent BBM as shown in any one of FIGS. 1AA-1AH, where each of X, A, and B is an ABM1 and Y is an ABM2 (this configuration of ABMs designated as “Tv 13” for convenience).

The disclosure further provides a tetravalent BBM as shown in any one of FIGS. 1AA-1AH, where each of Y, A, and B is an ABM1 and X is an ABM2 (this configuration of ABMs designated as “Tv 14” for convenience).

### 7.2.4. Trispecific Binding Molecule Configurations

Exemplary TBM configurations are shown in FIG. 2. FIG. 2A shows the components of the TBM configurations shown in FIGS. 2B-1V. The scFv, Fab, non-immunoglobulin based ABM, and Fc each can have the characteristics described for these components in Sections 7.2.1 and 7.2.2. The components of the TBM configurations shown in FIG. 2 can be associated with each other by any of the means described in Sections 7.2.1 and 7.2.2 (e.g., by direct bonds, ABM linkers, disulfide bonds, Fc domains with modified with knob in hole interactions, etc.). The orientations and associations of the various components shown in FIG. 2 are merely exemplary; as will be appreciated by a skilled artisan, other orientations and associations can be suitable (e.g., as described in Sections 7.2.1 and 7.2.2).

TBMs are not limited to the configurations shown in FIG. 2. Other configurations that can be used are known to those skilled in the art. See, e.g., WO 2014/145806; WO 2017/124002; Liu et al., 2017, Front Immunol. 8:38; Brinkmann & Kontermann, 2017, mAbs 9:2, 182-212; US 2016/0355600; Klein et al., 2016, MAbs 8(6):1010-20; and US 2017/0145116.

### 7.2.4.1. Exemplary Trivalent TBMs

TBMs can be trivalent, i.e., they have three antigen-binding domains, one of which binds CD19, one of which binds a component of a TCR complex, and one of which binds either CD2 or a TAA.

Exemplary trivalent TBM configurations are shown in FIGS. 2B through 2P.

As depicted in FIGS. 2B-2K and 2N-2P, a TBM can comprise two half antibodies, one comprising two ABMs and the other comprising one ABM, the two halves paired through an Fc domain.

In the embodiment of FIG. 2B, the first (or left) half antibody comprises an scFv and an Fc region, and the second (or right) half antibody comprises a Fab, an scFv and an Fc region. The first and second half antibodies are associated through the Fc regions forming an Fc domain.

In the embodiment of FIG. 2C, the first (or left) half antibody comprises two Fab and an Fc region, and the second (or right) half antibody comprises a Fab and an Fc region. The first and second half antibodies are associated through the Fc regions forming an Fc domain.

In the embodiment of FIG. 2D, the first (or left) half antibody comprises a Fab, an scFv and an Fc region, and the second (or right) half antibody comprises a Fab and an Fc region. The first and second half antibodies are associated through the Fc regions forming an Fc domain.

In the embodiment of FIG. 2E, the first (or left) half antibody comprises an scFv and an Fc region, and the second (or right) half antibody comprises two Fab and an Fc region. The first and second half antibodies are associated through the Fc regions forming an Fc domain.

In the embodiment of FIG. 2F, the first (or left) half antibody comprises an scFv, an Fc region, and a Fab, and the second (or right) half antibody comprises a Fab and an Fc region. The first and second half antibodies are associated through the Fc regions forming an Fc domain.

In the embodiment of FIG. 2G, the first (or left) half antibody comprises an scFv and an Fc region, and the second (or right) half antibody comprises a Fab an Fc region, and an scFV. The first and second half antibodies are associated through the Fc regions forming an Fc domain.

In the embodiment of FIG. 2H, the first (or left) half antibody comprises two Fab and an Fc region, and the second (or right) half antibody comprises a non-immunoglobulin based ABM and an Fc region. The first and second half antibodies are associated through the Fc regions forming an Fc domain.

In the embodiment of FIG. 21, the first (or left) half antibody comprises a Fab, an scFv, and an Fc region, and the second (or right) half antibody comprises a non-immunoglobulin based ABM and an Fc region. The first and second half antibodies are associated through the Fc regions forming an Fc domain.

In the embodiment of FIG. 2J, the first (or left) half antibody comprises a Fab and an Fc region, and the second (or right) half antibody comprises an scFv, a non-immunoglobulin based ABM and an Fc region. The first and second half antibodies are associated through the Fc regions forming an Fc domain.

In the embodiment of FIG. 2K, the first (or left) half antibody comprises an scFv and an Fc region, and the second (or right) half antibody comprises an scFv, an Fc region, and a second scFv. The first and second half antibodies are associated through the Fc regions forming an Fc domain.

In the embodiment of FIG. 2N, the first (or left) half antibody comprises a Fab, an Fc region, and an scFv, and the second (or right) half antibody comprises a Fab, and an Fc region. The first and second half antibodies are associated through the Fc regions forming an Fc domain.

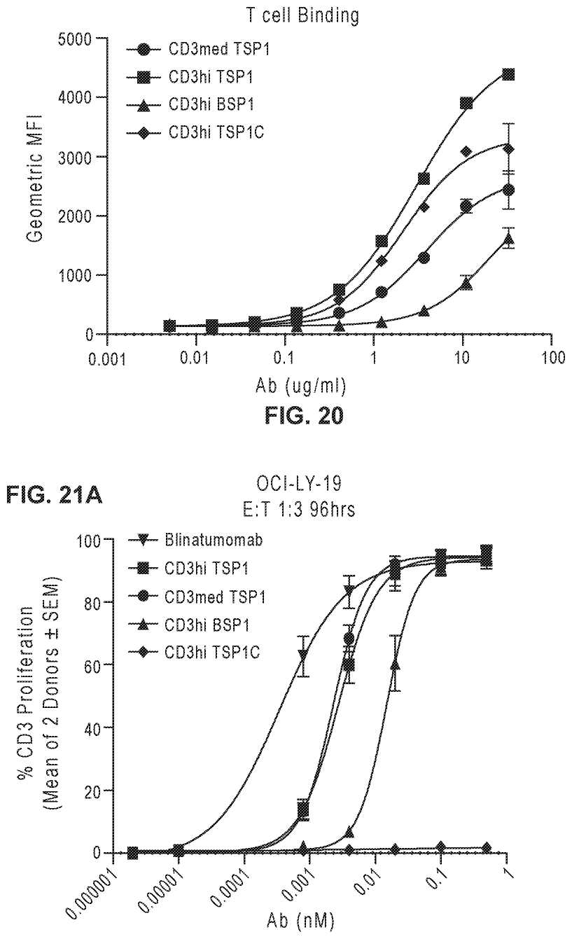

In the embodiment of FIG. 20, the first (or left) half antibody comprises a Fab, an Fc region, and a scFab, and the second (or right) half antibody comprises a Fab and an Fc region. The first and second half antibodies are associated through the Fc regions forming an Fc domain.

In the embodiment of FIG. 2P, the first (or left) half antibody comprises a Fab, a non-immunoglobulin based ABM, and an Fc region, and the second (or right) half antibody comprises a scFv and an Fc region. The first and second half antibodies are associated through the Fc regions forming an Fc domain.

Alternatively, as depicted in FIG. 2L, trivalent a TBM can comprise two half antibodies, each comprising one complete ABM and a portion of another ABM (one a VH, the other a VL). The two half antibodies are paired through an Fc domain, whereupon the VH and the VL associate to form a complete antigen-binding Fv domain.

The TBM can be a single chain, as shown in FIG. 2M. The TBM of FIG. 2M comprises three scFv domains connected through linkers.

In each of the configurations shown in FIGS. 2B-2P, each of the domains designated X, Y, and Z represents an ABM1, ABM2, or ABM3, although not necessarily in that order. In other words, X can be ABM1, ABM2, or ABM3, Y can be ABM1, ABM2, or ABM3, and Z can be ABM1, ABM2, or ABM3, provided that the TBM comprises one ABM1, one ABM2, and one ABM3.

Accordingly, in the present disclosure provides a trivalent TBM as shown in any one of FIGS. 2B through 2P, where X is an ABM1, Y is an ABM3 and Z is an ABM2 (this configuration of ABMs designated as “T1” for convenience).

The present disclosure also provides a trivalent TBM as shown in any one of FIGS. 2B through 2P, where X is an ABM1, Y is an ABM2, and Z is an ABM3 (this configuration of ABMs designated as “T2” for convenience).

The present disclosure further provides a trivalent TBM as shown in any one of FIGS. 2B through 2P, where X is an ABM3, Y is an ABM1, and Z is an ABM2 (this configuration of ABMs designated as “T3” for convenience).

The present disclosure yet further provides a trivalent TBM as shown in any one of FIGS. 2B through 2P, where X is an ABM3, Y is an ABM2, and Z is an ABM1 (this configuration of ABMs designated as “T4” for convenience).

The present disclosure yet further provides a trivalent TBM as shown in any one of FIGS. 2B through 2P, where X is an ABM2, Y is an ABM1, and Z is an ABM3 (this configuration of ABMs designated as “T5” for convenience).

The present disclosure yet further provides a trivalent TBM as shown in any one of FIGS. 2B through 2P, where X is an ABM2, Y is an ABM3, and Z is an ABM1 (this configuration of ABMs designated as “T6” for convenience).

### 7.2.4.2. Exemplary Tetravalent TBMs

The TBMs of the disclosure can be tetravalent, i.e., they have four antigen-binding domains, one or two of which binds CD19, one or two of which binds a component of a TCR complex, and one or two of which binds CD2 or a TAA.

Exemplary tetravalent TBM configurations are shown in FIGS. 2Q-2S.

As depicted in FIGS. 2Q-2S, a tetravalent TBM can comprise two half antibodies, each comprising two complete ABMs, the two halves paired through an Fc domain.

In the embodiment of FIG. 2Q, the first (or left) half antibody comprises a Fab, an Fc region, and a second Fab, and the second (or right) half antibody comprises a Fab, an Fc region, and a second Fab. The first and second half antibodies are associated through the Fc regions forming an Fc domain.

In the embodiment of FIG. 2R, the first (or left) half antibody comprises a Fab, an Fc region, and an scFv, and the second (or right) half antibody comprises a Fab, an Fc region, and an scFv. The first and second half antibodies are associated through the Fc regions forming an Fc domain.

In the embodiment of FIG. 2S, the first (or left) half antibody comprises a Fab, an Fc region, and an scFv, and the second (or right) half antibody comprises an scFv, an Fc region, and a Fab. The first and second half antibodies are associated through the Fc regions forming an Fc domain.

In the configuration shown in FIGS. 2Q-2S, each of X, Y, Z, and A represent an ABM1, an ABM2, or an ABM3, although not necessarily in that order, and provided that the TBM comprises at least one ABM1, at least one ABM2, and at least one ABM3. Thus, the tetravalent ABMs will include two ABMs against one of CD19, a component of a TCR complex, and CD2 or a TAA. In some cases, a tetravalent TBM has two CD19 ABMs.

### 7.2.4.3. Exemplary Pentavalent TBMs

The TBMs of the disclosure can be pentavalent, i.e., they have five antigen-binding domains, one, two, or three of which binds CD19, one, two, or three of which binds a component of a TCR complex, and one, two, or three of which binds CD2 or a TAA.

An exemplary pentavalent TBM configuration is shown in FIG. 2T.

As depicted in FIG. 2T, a pentavalent TBM can comprise two half antibodies, one of which comprises two complete ABMs and the other of which comprises one complete ABM, the two halves paired through an Fc domain.

In the embodiment of FIG. 2T, the first (or left) half antibody comprises a Fab, an scFv, and an Fc region, and the second (or right) half antibody comprises a Fab, an Fc region, and an scFv. The first and second half antibodies are associated through the Fc regions forming an Fc domain.

In the configuration shown in FIG. 2T, each of X, Y, Z, A, and B represent an ABM1, an ABM2, or an ABM3, although not necessarily in that order, and provided that the TBM comprises at least one ABM1, one ABM2, and one ABM3. Thus, the pentavalent TBMs can include two ABMs against two of CD19, a component of a TCR complex, and CD2 or a TAA, or three ABMs against one of CD19, a component of a TCR complex, and CD2 or a TAA. In some cases, a pentavalent TBM has two or three CD19 ABMs. In some embodiments, a pentavalent TBM has three ABM1s, one ABM2 and one ABM3.

### 7.2.4.4. Exemplary Hexavalent TBMs

The TBMs of the disclosure can be hexavalent, i.e., they have six antigen-binding domains, one, two, three, or four of which binds CD19, one, two, three, or four of which binds a component of a TCR complex, and one, two, three, or four of which binds CD2 or a TAA.

Exemplary hexavalent TBM configurations are shown in FIGS. 2U-2V.

As depicted in FIGS. 2U-2V, a pentavalent TBM can comprise two half antibodies, one of which comprises two complete ABMs and the other of which comprises one complete ABM, the two halves paired through an Fc domain.

In the embodiment of FIG. 2U, the first (or left) half antibody comprises a Fab, a second Fab, an Fc region, and an scFv, and the second (or right) half antibody comprises a Fab, a second Fab, an Fc region, and an scFv. The first and second half antibodies are associated through the Fc regions forming an Fc domain.

In the embodiment of FIG. 2V, the first (or left) half antibody comprises a first Fv, a second Fv, a third Fv, and an Fc region, and the second (or right) half antibody comprises a first Fv, a second Fv, a third Fv, and an Fc region. The first and second half antibodies are associated through the Fc regions forming an Fc domain.

In the configuration shown in FIGS. 2U-2V, each of X, Y, Z, A, B, and C represent an ABM1, an ABM2, or an ABM3, although not necessarily in that order, and provided that the TBM comprises at least one ABM1, one ABM2, and one ABM3. Thus, the hexavalent TBMs can include (i) two ABMs against each of CD19, a component of a TCR complex, and CD2 or a TAA, (ii) three ABMs against one of CD19, a component of a TCR complex, and CD2 or a TAA, or (iii) four ABMs against one of CD19, a component of a TCR complex, and CD2 or a TAA. For example, a hexavalent ABM can include three ABMs against CD19, two ABMs against CD2 or a TAA and one ABM against a component of a TCR complex. As another example, a hexavalent ABM can include three ABMs against CD19, two ABMs against a component of a TCR complex and one ABM against CD2 or a TAA. In some cases, a hexavalent TBM has two, three, our four CD19 ABMs. In some embodiments, a hexavalent TBM has three CD19 ABMs. In other embodiments, a hexavalent TBM has four CD19 ABMs.

### 7.2.5. TCR ABMs

The MBMs of the disclosure contain an ABM that specifically binds to CD19 and an ABM2 which is specific for a different antigen. In the BBMs, Type 1 TBMs and Type 2 TBMs of the disclosure, ABM2 can bind to a component of a TCR complex. The TCR is a disulfide-linked membrane-anchored heterodimeric protein normally consisting of the highly variable alpha (α) and beta (β) chains expressed as part of a complex with the invariant CD3 chain molecules. T cells expressing this receptor are referred to as α:β (or αβ) T cells, though a minority of T cells express an alternate receptor, formed by variable gamma (γ) and delta (δ) chains, referred as γδ T cells.

In an embodiment, MBMs contain an ABM that specifically binds to CD3.

### 7.2.5.1. CD3 ABMs

The MBMs can contain an ABM that specifically binds to CD3. The term “CD3” refers to the cluster of differentiation 3 co-receptor (or co-receptor complex, or polypeptide chain of the co-receptor complex) of the T cell receptor. The amino acid sequence of the polypeptide chains of human CD3 are provided in NCBI Accession P04234, P07766 and P09693. CD3 proteins can also include variants. CD3 proteins can also include fragments. CD3 proteins also include post-translational modifications of the CD3 amino acid sequences. Post-translational modifications include, but are not limited to, N- and O-linked glycosylation.

In some embodiments, a MBM can comprise an ABM which is an anti-CD3 antibody (e.g., as described in US 2016/0355600, WO 2014/110601, WO 2014/145806, or WO 2020/052692) or an antigen-binding domain thereof. Exemplary anti-CD3 VH, VL, and scFV sequences that can be used in a MBM are provided in Table 9A. Further exemplary anti-CD3 VH, VL, and scFv sequences that can be used in a MBM are provided in WO 2019/104075, WO 2019/195535, and WO 2020/052692, the contents of which are incorporated herein by reference in their entireties.

TABLE 9A
CD3 Binders-Variable domain sequences

| | | | SEQ |
|---|---|---|---|
| Binding | | | ID |
| Domain | Chain | Sequence | NO: |
| CD3hi | VH | EVQLVESGGGLVQPGGSLKLSCAASGFTFNTYAMNWVRQASGKGL | 1279 |
| | | EWVGRIRSKYNNYATYYADSVKDRFTISRDDSKSTLYLQMNSLKTE | |
| | | DTAVYYCVRHGNFGNSYVSWFAYWGQGTLVTVSS | |
| | VL | QAVVTQEPSLTVSPGGTVTLTCRSSTGAVTTSNYANWVQQKPGQA | 1280 |
| | | PRGLIGGTNKRAPWTPARFSGSLLGDKAALTLSGAQPEDEAEYFCA | |
| | | LWYSNLWVFGGGTKLTVL | |
| | scFv | EVQLVESGGGLVQPGGSLKLSCAASGFTFNTYAMNWVRQASGKGL | 1281 |
| | | EWVGRIRSKYNNYATYYADSVKDRFTISRDDSKSTLYLQMNSLKTE | |
| | | DTAVYYCVRHGNFGNSYVSWFAYWGQGTLVTVSSGGGGSGGGG | |
| | | SGGGGSGGGGSQAVVTQEPSLTVSPGGTVTLTCRSSTGAVTTSNY | |
| | | ANWVQQKPGQAPRGLIGGTNKRAPWTPARFSGSLLGDKAALTLSG | |
| | | AQPEDEAEYFCALWYSNLWVFGGGTKLTVL | |
| CD3med | VH | EVQLVESGGGLVQPGGSLKLSCAASGFTFNTYAMNWVRQASGKGL | 1282 |
| | | EWVGRIRSKYNNYATYYADSVKDRFTISRDDSKNTAYLQMNSLKTE | |
| | | DTAVYYCVRHGNFGNSYVSWFAHWGQGTLVTVSS | |
| | VL | QAVVTQEPSLTVSPGGTVTLTCGSSTGAVTSSNYANWVQQKPGQA | 1283 |
| | | PRGLIGGTNKRAPWTPARFSGSLLGGKAALTLSGAQPEDEAEYYCA | |
| | | LWYSNLWVFGGGTKLTVL | |
| | scFv | EVQLVESGGGLVQPGGSLKLSCAASGFTFNTYAMNWVRQASGKGL | 1284 |
| | | EWVGRIRSKYNNYATYYADSVKDRFTISRDDSKNTAYLQMNSLKTE | |
| | | DTAVYYCVRHGNFGNSYVSWFAHWGQGTLVTVSSGGGGSGGGG | |
| | | SGGGGSGGGGSQAVVTQEPSLTVSPGGTVTLTCGSSTGAVTSSNY | |
| | | ANWVQQKPGQAPRGLIGGTNKRAPWTPARFSGSLLGGKAALTLSG | |
| | | AQPEDEAEYYCALWYSNLWVFGGGTKLTVL | |
| CD3lo | VH | EVQLVESGGGLVQPGGSLKLSCAASGFTFNTYAMNWVRQASGKGL | 1285 |
| | | EWVGRIRSKYNNYATYYADSVKDRFTISRDDSKSTAYLQMNSLKTE | |
| | | DTAVYYCVRHGNFGNSYVSWFAYWGQGTLVTVSS | |
| | VL | QAVVTQEPSLTVSPGGTVTLTCRSSTGAVTTSNYANWVQQKPGQA | 1280 |
| | | PRGLIGGTNKRAPWTPARFSGSLLGDKAALTLSGAQPEDEAEYFCA | |
| | | LWYSNLWVFGGGTKLTVL | |
| | scFv | EVQLVESGGGLVQPGGSLKLSCAASGFTFNTYAMNWVRQASGKGL | 1286 |
| | | EWVGRIRSKYNNYATYYADSVKDRFTISRDDSKSTAYLQMNSLKTE | |
| | | DTAVYYCVRHGNFGNSYVSWFAYWGQGTLVTVSSGGGGSGGGG | |
| | | SGGGGSGGGGSQAVVTQEPSLTVSPGGTVTLTCRSSTGAVTTSNY | |
| | | ANWVQQKPGQAPRGLIGGTNKRAPWTPARFSGSLLGDKAALTLSG | |
| | | AQPEDEAEYFCALWYSNLWVFGGGTKLTVL | |

CDR sequences for CD3hi, CD3med, and CD31o as defined by the Kabat numbering scheme (Kabat et al, 1991, Sequences of Proteins of Immunological Interest, 5^{th} Ed. Public Health Service, National Institutes of Health, Bethesda, Md) are provided in Table 9B.

TABLE 9B
CD3 Binders-CDR sequences according to Kabat numbering scheme

| Binding | | | SEQ ID | | SEQ ID | | SEQ ID |
|---|---|---|---|---|---|---|---|
| Domain | Chain | CDR1 | NO: | CDR2 | NO: | CDR3 | NO: |
| CD3hi | VH | TYAMN | 1287 | RIRSKYNNYATYYA | 1290 | HGNFGNSYVS | 1292 |
| | | | | DSVKD | | WFAY | |
| | VL | RSSTGAVTT | 1288 | GTNKRAP | 1291 | ALWYSNLWV | 1293 |
| | | SNYAN | | | | | |
| CD3med | VH | TYAMN | 1287 | RIRSKYNNYATYYA | 1290 | HGNFGNSYVS | 1294 |
| | | | | DSVKD | | WFAH | |
| | VL | GSSTGAVTS | 1289 | GTNKRAP | 1291 | ALWYSNLWV | 1293 |
| | | SNYAN | | | | | |
| CD3lo | VH | TYAMN | 1287 | RIRSKYNNYATYYA | 1290 | HGNFGNSYVS | 1292 |
| | | | | DSVKD | | WFAY | |
| | VL | RSSTGAVTT | 1288 | GTNKRAP | 1291 | ALWYSNLWV | 1293 |
| | | SNYAN | | | | | |

In some embodiments, a MBM can comprise a CD3 ABM which comprises the CDRs of any of CD3hi, CD3med, or CD31o as set forth in Table 9B.

In some embodiments, a MBM comprises a CD3 ABM which comprises the VH and VL sequences of CD3hi. In some embodiments, a MBM comprises a CD3 ABM which comprises the VH and VL sequences of CD3med. In some embodiments, a MBM comprises a CD3 ABM which comprises the VH and VL sequences of CD31o.

In addition to the CDR sets described in Table 9B (i.e., the set of six CDRs for each of CD3hi, CD3med, and CD3lo), the present disclosure provides variant CDR sets. In one embodiment, a set of 6 CDRs can have 1, 2, 3, 4 or 5 amino acid changes from a CDR set described in Table 9B, as long as the CD3 ABM is still able to bind to the target antigen, as measured by at least one of a Biacore, surface plasmon resonance (SPR) and/or BLI (biolayer interferometry, e.g., Octet assay) assay.

In addition to the variable heavy and variable light domains disclosed in Table 9A that form an ABM to CD3, the present disclosure provides variant VH and VL domains. In one embodiment, the variant VH and VL domains each can have from 1, 2, 3, 4, 5, 6, 7, 8, 9 or 10 amino acid changes from the VH and VL domain set forth in Table 9A, as long as the ABM is still able to bind to the target antigen, as measured at least one of a Biacore, surface plasmon resonance (SPR) and/or BLI (biolayer interferometry, e.g., Octet assay) assay. In another embodiment, the variant VH and VL are at least 90, 95, 97, 98 or 99% identical to the respective VH or VL disclosed in Table 9A, as long as the ABM is still able to bind to the target antigen, as measured by at least one of a Biacore, surface plasmon resonance (SPR) and/or BLI (biolayer interferometry, e.g., Octet assay) assay.

VH and VL sequences (amino acid sequences and the nucleotide sequences encoding the amino acid sequences) can be “mixed and matched” to create other CD3 ABMs. Such “mixed and matched” CD3 ABMs can be tested using binding assays known in the art (e.g., FACS assays). When chains are mixed and matched, a VH sequence from a particular VH/VL pairing should be replaced with a structurally similar VH sequence. A VL sequence from a particular VH/VL pairing should be replaced with a structurally similar VL sequence.

In some embodiments, the antigen-binding domain that specifically binds to human CD3 is non-immunoglobulin based and is instead derived from a non-antibody scaffold protein, for example one of the non-antibody scaffold proteins described in Section 7.2.1.2. In an embodiment, the antigen-binding domain that specifically binds to human CD3 comprises Affilin-144160, which is described in WO 2017/013136. Affilin-144160 has the following amino acid sequence:

(SEQ ID NO: 1295)
MQIFVKTLTGKTITLEVEPSDTIENVKAKIQDKEGIPPDQQWLWFAGKQ
LEDGRTLSDYNIQKESTLKLWLVDKAAMQIFVYTRTGKTITLEVEPSDT
IENVKAKIQDKEGIPPDQQRLIWAGKQLEDGRTLSDYNIALESGLHLVL
RLRAA

### 7.2.5.2. TCR-α/β ABMs

The MBMs can contain an ABM that specifically binds to the TCR-α chain, the TCR-β chain, or the TCR-αβ dimer. Exemplary anti-TCR-α/β antibodies are known (see, e.g., US 2012/0034221; Borst et al., 1990, Hum Immunol. 29(3):175-88 (describing antibody BMA031)). The VH, VL, and Kabat CDR sequences of antibody BMA031 are provided in Table 10.

TABLE 10
BMA031 sequences

| | | SEQ |
|---|---|---|
| | | ID |
| Domain | Sequence | NO: |
| BMA031 | KASGYKFTSYVMH | 1296 |
| CDR-H1 | | |
| BMA031 | YINPYNDVTKYNEKFK | 1297 |
| CDR-H2 | | |
| BMA031 | GSYYDYDGFVY | 1298 |
| CDR-H3 | | |
| BMA031 | SATSSVSYMH | 1299 |
| CDR-L1 | | |
| BMA031 | DTSKLAS | 1300 |
| CDR-L2 | | |
| BMA031 | QQWSSNPLT | 1301 |
| CDR-L3 | | |
| BMA031 | EVQLQQSGPELVKPGASVKMSCKASGYKFTSYVMHWVKQKPGQGLE | 1302 |
| VH | WIGYINPYNDVTKYNEKFKGKATLTSDKSSSTAYMELSSLTSEDSAVH | |
| | YCARGSYYDYDGFVYWGQGTLVTVSA | |
| BMA031 | QIVLTQSPAIMSASPGEKVTMTCSATSSVSYMHWYQQKSGTSPKRWI | 1303 |
| VL | YDTSKLASGVPARFSGSGSGTSYSLTISSMEAEDAATYYCQQWSSNP | |
| | LTFGAGTKLELK | |

In an embodiment, a TCR ABM can comprise the CDR sequences of antibody BMA031. In other embodiments, a TCR ABM can comprise the VH and VL sequences of antibody BMA031.

### 7.2.5.3. TCR-γ/δ ABMs

The MBMs can contain an ABM that specifically binds to the TCR-γ chain, the TCR-δ chain, or the TCR-γδ dimer. Exemplary anti-TCR-γ/δ antibodies are known (see, e.g., U.S. Pat. No. 5,980,892 (describing δTCS1, produced by the hybridoma deposited with the ATCC as accession number HE 9578)).

### 7.2.6. CD2 ABMs

### 7.2.6.1. Immunoglobulin-Based CD2 ABMs

A Type 1 TBM can comprise an ABM which is an anti-CD2 antibody or an antigen-binding domain thereof. Exemplary anti-CD2 antibodies are known (see, e.g., U.S. Pat. No. 6,849,258, CN102827281A, US 2003/0139579 A1, and U.S. Pat. No. 5,795,572). Table 11 provides exemplary CDR, VH, and VL sequences that can be included in ant-CD2 antibodies or antigen-binding fragments thereof, for use in MBMs of the disclosure.

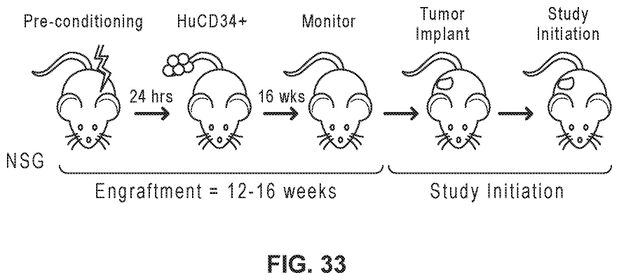

TABLE 11
Immunoglobulin Based CD2 Binders

| | | | SEQ ID |
|---|---|---|---|
| Name | Domain | Sequence | NO: |
| CD2-1 | CDR-H1 | EYYMY (Rat Lo-CD2a = BTI-322 from FIG. 33 of USP | 1304 |
| | | 6,849,258) | |
| CD2-1 | CDR-H2 | RIDPEDGSIDYVEKFKK (Rat Lo-CD2a = BTI-322 from FIG. | 1305 |
| | | 33 of USP 6,849,258) | |
| CD2-1 | CDR-H3 | GKFNYRFAY (Rat Lo-CD2a = BTI-322 from FIG. 33 of USP | 1306 |
| | | 6,849,258) | |
| CD2-1 | CDR-L1 | RSSQSLLHSSGNTYLN (Rat Lo-CD2a = BTI-322 from FIG. | 1307 |
| | | 31 of USP 6,849,258) | |
| CD2-1 | CDR-L2 | LVSKLES (Rat Lo-CD2a = BTI-322 from FIG. 31 of USP | 1308 |
| | | 6,849,258) | |
| CD2-1 | CDR-L3 | QFTHYPYT (Rat Lo-CD2a = BTI-322 from FIG. 31 of USP | 1309 |
| | | 6,849,258) | |
| CD2-1 | VH | EVQLQQSGPELQRPGASVKLSCKASGYIFTEYYMYWVKQR | 1225 |
| | | PKQGLELVGRIDPEDGSIDYVEKFKKKATLTADTSSNTAYM | |
| | | QLSSLTSEDTATYFCARGKFNYRFAYWGQGTLVTVSS | |
| | | (SEQ ID NO: 100 of USP 6,849,258) | |
| CD2-1 | VL | DVVLTQTPPTLLATIGQSVSISCRSSQSLLHSSGNTYLNWLL | 1216 |
| | | QRTGQSPQPLIYLVSKLESGVPNRFSGSGSGTDFTLKISGV | |
| | | EAEDLGVYYCMQFTHYPYTFGAGTKLELK (Rat Lo-CD2a Vk | |
| | | from SEQ ID NO: 92, without signal sequence as shown | |
| | | in FIG. 31 of USP 6,849,258) | |
| hu1CD2-1 | VH | QVQLVQSGAEVKKPGASVKVSCKASGYTFTEYYMYWVRQ | 1226 |
| | | APGQGLELMGRIDPEDGSIDYVEKFKKKVTLTADTSSSTAY | |
| | | MELSSLTSDDTAVYYCARGKFNYRFAYWGQGTLVTVSS | |
| | | (SEQ ID NO: 101 of USP 6,849,258) | |
| | VL | DVVMTQSPPSLLVTLGQPASISCRSSQSLLHSSGNTYLNWL | 1221 |
| | | LQRPGQSPQPLIYLVSKLESGVPDRFSGSGSGTDFTLKISG | |
| | | VEAEDVGVYYCMQFTHYPYTFGQGTKLEIK (SEQ ID NO: 96 | |
| | | of USP 6,849,258) | |
| hu2CD2-1 | VH | EVQLQQSGPELQRPGASVKLSCKASGYIFTEYYMYWVKQR | 1225 |
| | | PKQGLELVGRIDPEDGSIDYVEKFKKKATLTADTSSNTAYM | |
| | | QLSSLTSEDTATYFCARGKFNYRFAYWGQGTLVTVSS (Vh | |
| | | of MEDI-507; SEQ ID NO: 105 of USP 6,849,258) | |
| | VL | DVVMTQSPPSLLVTLGQPASISCRSSQSLLHSSGNTYLNWL | 1221 |
| | | LQRPGQSPQPLIYLVSKLESGVPDRFSGSGSGTDFTLKISG | |
| | | VEAEDVGVYYCMQFTHYPYTFGQGTKLEIK (SEQ ID NO: 96 | |
| | | of USP 6,849,258)(same as hu1CD2-1) | |

In some embodiments, a 002 ABM comprises the CDR sequences of 002-1 (SEQ ID NOS:). In some embodiments, a 002 ABM comprises the heavy and light chain variable sequences of 002-1 (SEQ ID NOS; and, respectively). In some embodiments, a 002 ABM comprises the heavy and light chain variable sequences of hu1CD2-1 (SEQ ID NOS; and, respectively). In some embodiments, a 002 ABM comprises the heavy and light chain variable sequences of hu2CD2-1 (SEQ ID NOS; and, respectively).

In other embodiments, a 002 ABM can comprise the CDR sequences of antibody 901 produced by the hybridoma deposited with the Chinese Culture Collection Committee General Microbiology Center on May 16, 2012 with accession no. CGMCC 6132, and which is described in CN102827281A. In other embodiments, a CD2 ABM can comprise the CDR sequences of antibody LO-CD2b produced by the hybridoma deposited with the American Type Culture Collection on Jun. 22, 1999 with accession no. PTA-802, and which is described in US 2003/0139579 A1. In yet other embodiments, a CD2 ABM can comprise the CDR sequences of the CD2 SFv-Ig produced by expression of the construct cloned in the recombinant E. coli deposited with the ATCC on Apr. 9, 1993 with accession no. 69277, and which is described in U.S. Pat. No. 5,795,572.

In other embodiments, a CD2 ABM can comprise the VH and VL sequences of antibody 9D1. In other embodiments, a CD2 ABM can comprise the VH and VL sequences of antibody LO-CD2b. In yet other embodiments, a CD2 ABM can comprise the VH and VL sequences of the CD2 SFv-Ig produced by expression of the construct cloned in the recombinant E. coli having ATCC accession no. 69277.

### 7.2.6.2. CD58-Based CD2 ABMs

In certain aspects the present disclosure provides a Type 1 TBM comprising a CD2 ABM which is a ligand. The CD2 ABM specifically binds to human CD2, whose natural ligand is CD58, also known as LFA-3. CD58/LFA-3 proteins are glycoproteins that are expressed on the surfaces of a variety of cell types (Dustin et al., 1991, Annu. Rev. Immunol. 9:27) and play roles in mediating T-cell interactions with APCs in both antigen-dependent and antigen-independent manners (Wallner et al., 1987, J. Exp. Med. 166:923). Accordingly, in certain aspects, the CD2 ABM is a CD58 moiety. As used herein, a CD58 moiety comprises an amino acid sequence comprising at least 70% sequence identity to a CD2-binding portion of CD58, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a CD2-binding portion of CD58. The sequence of human CD58 has the Uniprot identifier P19256 (www.uniprot.org/uniprot/P19256). It has been established that CD58 fragments containing amino acid residues 30-123 of full length CD58 (i.e., the sequence designated as CD58-6 in Table 12 below) are sufficient for binding to CD2. Wang et al., 1999, Cell 97:791-803. Accordingly, in certain aspects, a CD58 moiety comprises an amino acid sequence comprising at least 70% sequence identity to amino acids 30-123 of CD58, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to the amino acid sequence designated CD58-6.

The interactions between CD58 and CD2 have been mapped through x-ray crystallography and molecular modeling. The substitution of residues E25, K29, K30, K32, D33, K34, E37, D84 and K87 (with numbering referring to the in the mature polypeptide) reduces binding to 002. Ikemizu et al., 1999, Proc. Natl. Acad. Sci. USA 96:4289-94. Accordingly, in some embodiments the CD58 moiety retains the wild type residues at E25, K29, K30, K32, D33, K34, E37, D84 and K87.

In contrast, the following substitutions (with numbering referring to the full length polypeptide) did not impact binding to CD2: F29S; V37K; V49Q; V86K; T113S; and L121G. Accordingly, a CD58 moiety can include one, two, three, four, five or all six of the foregoing substitutions.

In some embodiments, the CD58 moiety is engineered to include a pair of cysteine substitutions that upon recombinant expression create a disulfide bridge. Exemplary amino acid pairs that can be substituted with cysteines in order to form a disulfide bridge upon expression (with numbering referring to the full length polypeptide) are (a) a V45C substitution and a M105C substitution; (b) a V54C substitution and a G88C substitution; (c) a V45C substitution and a M 114C substitution; and (d) a W56C substitution and a L90C substitution.

Exemplary 0058 moieties are provided in Table 12 below:

TABLE 12
CD58 sequences

| Name | Description | Sequence | SEQ ID NO: |
|---|---|---|---|
| CD58-1 | Full length CD58, | MVAGSDAGRALGVLSVVCLLHCFGFISCFSQQIYGVVY | 1310 |
| | including signal | GNVTFHVPSNVPLKEVLWKKQKDKVAELENSEFRAFS | |
| | sequence and full | SFKNRVYLDTVSGSLTIYNLTSSDEDEYEMESPNITDTM | |
| | intracellular domain | KFFLYVLESLPSPTLTCALTNGSIEVQCMIPEHYNSHRG | |
| | (P19256) | LIMYSWDCPMEQCKRNSTSIYFKMENDLPQKIQCTLSN | |
| | | PLFNTTSSIILTTCIPSSGHSRHRYALIPIPLAVITTCIVLY | |
| | | MNGILKCDRKPDRTNSN | |
| CD58-2 | Full length CD58, | MVAGSDAGRALGVLSVVCLLHCFGFISCFSQQIYGVVY | 1311 |
| | including signal | GNVTFHVPSNVPLKEVLWKKQKDKVAELENSEFRAFS | |
| | sequence and but | SFKNRVYLDTVSGSLTIYNLTSSDEDEYEMESPNITDTM | |
| | no intracellular | KFFLYVLESLPSPTLTCALTNGSIEVQCMIPEHYNSHRG | |
| | domain (P19256-2) | LIMYSWDCPMEQCKRNSTSIYFKMENDLPQKIQCTLSN | |
| | | PLFNTTSSIILTTCIPSSGHSRHRYALIPIPLAVITTCIVLY | |
| | | MNVL | |
| CD58-3 | Full length CD58, | MVAGSDAGRALGVLSVVCLLHCFGFISCFSQQIYGVVY | 1312 |
| | including signal | GNVTFHVPSNVPLKEVLWKKQKDKVAELENSEFRAFS | |
| | sequence and | SFKNRVYLDTVSGSLTIYNLTSSDEDEYEMESPNITDTM | |
| | variant intracellular | KFFLYVLESLPSPTLTCALTNGSIEVQCMIPEHYNSHRG | |
| | domain (P19256-3) | LIMYSWDCPMEQCKRNSTSIYFKMENDLPQKIQCTLSN | |
| | | PLFNTTSSIILTTCIPSSGHSRHRYALIPIPLAVITTCIVLY | |
| | | MNGILKCDRKPDRTK | |
| CD58-4 | Extracellular domain | FSQQIYGVVYGNVTFHVPSNVPLKEVLWKKQKDKVAE | 1313 |
| | of CD58, | LENSEFRAFSSFKNRVYLDTVSGSLTIYNLTSSDEDEYE | |
| | corresponding to | MESPNITDTMKFFLYVLESLPSPTLTCALTNGSIEVQCM | |
| | amino acids 29-215 | IPEHYNSHRGLIMYSWDCPMEQCKRNSTSIYFKMENDL | |
| | of CD58 (WT) | PQKIQCTLSNPLFNTTSSIILTTCIPSSGHSRHR | |
| CD58-5 | Extracellular domain | BSQQIYGVJYGNVTFHVPSNOPLKEVLWKKQKDK | 1278 |
| | of CD58, | VAELENSEFRAFSSFKNRVYLDTUSGSLTIYNLTS | |
| | corresponding to | SDEDEYEMESPNITDXMKFFLYVZESLPSPTLTCA | |
| | amino acids 29-215 | LTNGSIEVQCMIPEHYNSHRGLIMYSWDCPMEQC | |
| | of CD58 (with | KRNSTSIYFKMENDLPQKIQCTLSNPLFNTTSSIILT | |
| | permitted | TCIPSSGHSRHR | |
| | substitutions) | B = F or S | |
| | | J = V or K | |
| | | O = V or Q | |
| | | U = V or K | |
| | | X = T or S | |
| | | Z = L or G | |
| CD58-6 | Amino acids 30-123 | SQQIYGWYGNVTFHVPSNVPLKEVLWKKQKDKVAEL | 1315 |
| | (WT) | ENSEFRAFSSFKNRVYLDTVSGSLTIYNLTSSDEDEYE | |
| | Ig-V like domain | MESPNITDTMKFFLYVLES | |
| CD58-7 | Amino acids 30-123 | SQQIYGVJYGNVTFHVPSNOPLKEVLWKKQKDKVAEL | 1316 |
| | (with permitted | ENSEFRAFSSFKNRVYLDTVSGSLTIYNLTSSDEDEYE | |
| | substitutions) | MESPNITDTMKFFLYVLES | |
| | Ig-V like domain | J = V or K | |
| | | O = V or Q | |
| CD58-8 | Amino acids 30-123 | SQQIYGVVYGNVTFHCPSNVPLKEVLWKKQKDKVAEL | 1317 |
| | (V45C_M105C) | ENSEFRAFSSFKNRVYLDTVSGSLTIYNLTSSDEDEYE | |
| | Ig-V like domain | CESPNITDTMKFFLYVLES | |
| CD58-9 | Amino acids 30-123 | SQQIYGVVYGNVTFHVPSNVPLKECLWKKQKDKVAEL | 1318 |
| | (V54C_G88C) | ENSEFRAFSSFKNRVYLDTVSCSLTIYNLTSSDEDEYE | |
| | Ig-V like domain | MESPNITDTMKFFLYVLES | |
| CD58-10 | Amino acids 30-123 | SQQIYGVVYGNVTFHCPSNVPLKEVLWKKQKDKVAEL | 1319 |
| | (V45C_M114C) | ENSEFRAFSSFKNRVYLDTVSGSLTIYNLTSSDEDEYE | |
| | Ig-V like domain | MESPNITDTCKFFLYVLES | |
| CD58-11 | Amino acids 30-123 | SQQIYGWVYGNVTFHVPSNVPLKEVLCKKQKDKVAELE | 1320 |
| | (W56C_L90C) | NSEFRAFSSFKNRVYLDTVSGSCTIYNLTSSDEDEYEM | |
| | Ig-V like domain | ESPNITDTMKFFLYVLES | |

### 7.2.6.3. CD48-Based CD2 ABMs

In certain aspects the present disclosure provides a MBM comprising a CD2 ABM which is CD48 moiety. As used herein, a CD48 moiety comprises an amino acid sequence comprising at least 70% sequence identity to a CD2-binding portion of CD48, e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity to a CD2-binding portion of CD48. The sequence of human CD48 has the Uniprot identifier P09326 (www.uniprot.org/uniprot/P09326), which includes a signal peptide (amino acids 1-26) and a GPI anchor (amino acids 221-243). In certain aspects, a CD48 moiety comprises an amino acid sequence comprising at least 70% sequence identity (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to the amino acid sequence consisting of amino acids 27-220 of Uniprot identifier P09326. Human CD48 has an Ig-like C2-type I domain (amino acids 29-127 of Uniprot identifier P09326) and a Ig-like C2 type 2 domain (amino acids 132-212 of Uniprot identifier P09326). Accordingly, in some embodiments, a CD48 moiety comprises an amino acid sequence comprising at least 70% sequence identity (e.g., at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity) to the amino acid sequence consisting of amino acids 29-212 of Uniprot identifier P09326, to the C2-type I domain (amino acids 29-127 of Uniprot identifier P09326) and/or to the Ig-like C2 type 2 domain (amino acids 132-212 of Uniprot identifier P09326). A CD48 moiety can in some embodiments comprise one or more natural variants relative to the sequence of Uniprot identifier P09326. For example, a CD48 moiety can include a E102Q substitution. As another example, a CD48 moiety can comprise an amino acid sequence corresponding to a CD-48 isoform or a CD2 binding portion thereof, e.g., the isoform having Uniprot identifier P09326-2 or a CD2 binding portion thereof.

### 7.2.7. Tumor-Associated Antigen ABMs

The Type 2 TBMs can comprise an ABM that binds specifically to a tumor-associated antigen (TAA). In some embodiments, the TAA is a human TAA. The antigen may or may not be present on normal cells. In certain embodiments, the TAA is preferentially expressed or upregulated on tumor cells as compared to normal cells. In other embodiments, the TAA is a lineage marker.

In certain embodiments, the TAA is expressed or upregulated on cancerous B cells as compared to normal B cells. In other embodiments, the TAA is a B cell lineage marker.

It is anticipated that any type of B cell malignancy can be targeted by the MBMs of the disclosure. Exemplary types of B cell malignancies that can be targeted include Hodgkin's lymphomas, non-Hodgkin's lymphomas (NHLs), and multiple myeloma. Examples of NHLs include diffuse large B-cell lymphoma (DLBCL), follicular lymphoma, chronic lymphocytic leukemia (CLL)/small lymphocytic lymphoma (SLL), mantle cell lymphoma (MCL), marginal zone lymphomas, Burkitt lymphoma, lymphoplasmacytic lymphoma (Waldenstrom macroglobulinemia), hairy cell leukemia, splenic marginal zone B-cell lymphoma, extranodal marginal zone B-cell lymphoma of MALT, nodal marginal zone B-cell lymphoma, and primary effusion lymphoma.

Examples of TAAs that can be targeted by CD19-binding MBMs (e.g., TBMs) include BCMA, CD20, CD22, CD123, CD33, CLL1, CD138 (also known as Syndecan-1, SDC1), CS1, CD38, CD133, FLT3, CD52, TNFRSF13C (TNF Receptor Superfamily Member 13C, also referred to in the art as BAFFR: B-Cell-Activating Factor Receptor), TNFRSF13B (TNF Receptor Superfamily Member 13B, also referred to in the art as TACI: Transmembrane Activator And CAML Interactor), CXCR4 (C—X—C Motif Chemokine Receptor 4), PD-L1 (programmed death-ligand 1), LY9 (lymphocyte antigen 9, also referred to in the art as CD229), CD200, FCGR2B (Fc fragment of IgG receptor IIb, also referred to in the art as CD32b), CD21, CD23, CD24, CD40L, CD72, CD79a, and CD79b. In some embodiments, the TAA is BCMA. In some embodiments, the TAA is CD20. In some embodiments, the TAA is CD22. In some embodiments, the TAA is CD123. In some embodiments, the TAA is CD33. In some embodiments, the TAA is CLL1. In some embodiments, the TAA is CD138. In some embodiments, the TAA is CS1. In some embodiments, the TAA is CD38. In some embodiments, the TAA is CD133. In some embodiments, the TAA is FLT3. In some embodiments, the TAA is CD52. In some embodiments, the TAA is TNFRSF13C. In some embodiments, the TAA is TNFRSF13B. In some embodiments, the TAA is CXCR4. In some embodiments, the TAA is PD-L1. In some embodiments, the TAA is LY9. In some embodiments, the TAA is CD200. In some embodiments, the TAA is CD21. In some embodiments, the TAA is CD23. In some embodiments, the TAA is CD24. In some embodiments, the TAA is CD40L. In some embodiments, the TAA is CD72. In some embodiments, the TAA is CD79a. In some embodiments, the TAA is CD79b.

A TAA-binding ABM can comprise, for example, an anti-TAA antibody or an antigen-binding fragment thereof. The anti-TAA antibody or antigen-binding fragment can comprise, for example, the CDR sequences of an antibody set forth in Table 15. In some embodiments, the anti-TAA antibody or antigen-binding domain thereof has the heavy and light chain variable region sequences of an antibody set forth in Table 15.

TABLE 15
Exemplary Anti-Tumor-Associated Antigen Antibodies

| Target | ExampIes of Antibody Name and/or Reference(s) and/or Source |
|---|---|
| CD123 | Any CD123 antibody described in U.S. Pat. No. 8,852,551, EP2426148, WO |
| | 2014/138819, WO 2016/028896, or WO 2014/130635 |
| BCMA | Any BCMA antibody described in WO2012163805, WO200112812, or |
| | WO2003062401. |
| CD20 | Rituximab, Ofatumumab, Ocrelizumab, Veltuzumab, or GA101 |
| CD22 | Any CD22 antibody described in Haso et al., 2013, Blood, 121(7): 1165-1174, |
| | Wayne et al., 2010, Clin Cancer Res 16(6): 1894-1903, Kato et al., 2013, Leuk |
| | Res 37(1): 83-88, or Creative BioMart (creativebiomart.net): MOM-18047-S(P). |
| CD33 | Any CD33 antibody described in Bross et al., 2001, Clin Cancer Res 7(6): 1490- |
| | 1496 (Gemtuzumab Ozogamicin, hP67.6), Caron et al., 1992, Cancer Res |
| | 52(24): 6761-6767 (Lintuzumab, HuM195), Lapusan et al., 2012, Invest New |
| | Drugs 30(3): 1121-1131 (AVE9633), Aigner et al., 2013, Leukemia 27(5): 1107- |
| | 1115 (AMG330, CD33 BiTE), Dutour et al., 2012, Adv Hematol 2012: 683065, or |
| | Pizzitola et al., 2014, Leukemia doi: 10.1038/Lue.2014.62. |
| CD38 | Daratumumab (see, e.g., Groen et al., 2010, Blood 116(21): 1261-1262; MOR202 |
| | (see, e.g., U.S. Pat. No. 8,263,746); or any CD38 antibody described in U.S. Pat. |
| | No. 8,362,211. |
| CLL-1 | PE-CLL1-hu Cat# 353604 (BioLegend); PE-CLL1 (CLEC12A) Cat# 562566 |
| | (BD); Any CLL-1 antibody described in WO 2014/051433 A1, US 2016/0368994 |
| | A1, US 2013/0295118 A1, U.S. Pat. No. 8,536,310 B2, Lu et al., 2014, |
| | Angewandte Chemie International Edition 53(37): 9841-9845, or Leong et al., |
| | 2017, Blood 129(5): 609-618 |
| CS1 | Elotuzumab (BMS), see e.g., Tai et al., 2008, Blood 112(4): 1329-37; Tai et al., |
| | 2007, Blood. 110(5): 1656-63. |
| FLT3 | Any FLT3 antibody described in WO 2011/076922, U.S. Pat. No. 5777084, |
| | EP0754230, or US 2009/0297529. |
| CD133 | Any CD133 antibody described in U.S. Pat No. 9,624,303, WO 2016/154623, or |
| | WO 2011/089211; 5E3 (ThermoFisher); MAB11331 (R&D Systems); MAB4310 |
| | (Millipore Sigma) |
| CD138 | Any CD138 antibody described in WO/2009/080829, WO/2017/014679, or U.S. |
| | Pat. No. 9,289,509; nBT062 (Biotest AG); MI15, B-A38, SP152, DL-101 |
| | (ThermoFisher) |
| CD52 | alemtuzumab (Genzyme); ANT1034 (see, Holgate et al., 2015, PLOS ONE |
| | 10(9): e0138123; any CD52 antibody described in WO/2010/132659; any CD52 |
| | antibody described in U.S. Pat No. 9,708,407; any CD52 antibody described in |
| | WO/2010/132659 |
| TNFRSF13C | Any TNFRSF13C antibody described in WO 2010/007082, U.S. Pat. No. |
| | 9,382,326 |
| TNFRSF13B | Any TNFRSF13B antibody described in WO 2004/011611; LS-C89973 (Lifespan |
| | Biosciences, Inc.) M02952-1 (Boster Biological Technology); MAB1041, |
| | MAB1741, and MAB174 (R&D Systems) |
| CXCR4 | Any CXCR4 antibody described in U.S. Pat. Nos. 7,138,496, 8,329,178, |
| | 8,450,464, 9,249,223, or 9,260,527 |
| PD-L1 | Any PD-L1 antibody described in US 2015/0203580, US 2017/0058033, US |
| | 2017/0204184, U.S. Pat. No. 8,741,295, U.S. Pat. No. 9,789,183, or U.S. Pat. No. |
| | 9,637,546 |
| LY9 | HLy9.25 (e.g., Lifespan Biosciences, Inc. cat. no. LS-C112605); MAB1898 (R&D |
| | Systems) |
| CD200 | Any CD200 antibody described in U.S. Pat. No. 7,887,798; ab23552 (Abcam); |
| | Ox104 (ThermoFisher) |
| FCGR2B | Any FCGR2B antibody described in U.S. Pat No. 8,802,089 or WO 2017/103895; |
| | ab45143 (Abcam); AT130-2 (ThermoFisher); 2E10 (Millipore Sigma) |
| CD21 | ab75985 (Abcam); ab9492 (Abcam); 2G9 (ThermoFisher); HB5 (ThermoFisher); |
| | MAB4909 (R&D Systems) |
| CD23 | Any CD23 antibody described in U.S. Pat. No. 7,008,623 or U.S. Pat. No. |
| | 6,011,138; lumiliximab (Biogen); ab16702 (Abcam); SP23 (ThermoFisher) |
| CD24 | Any CD24 antibody described in U.S. Pat. No. 8,614,301; SN3 (ThermoFisher); |
| | SN3b (ThermoFisher); 2Q1282 (Santa Cruz Biotechnology); 3H1143 (Santa |
| | Cruz Biotechnology); ALB9 (Santa Cruz Biotechnology); MAB5248 (R&D |
| | Systems) |
| CD40L | Any CD40L antibody described in U.S. Pat. No. 9,228,018 or US 2003/0099642; |
| | 24-31 (Biolegend); ab52750 (Abcam); ab47204 (Abcam); CDP7657 (UCB |
| | Pharma); 5c8 (Biogen) |
| CD72 | 3F3 (Biolegend); Bu40 (ThermoFisher); H-7 (Santa Cruz Biotechnology); H-96 |
| | (Santa Cruz Biotechnology); G-5 (Santa Cruz Biotechnology); ab92509 (Abcam) |
| CD79a | ab62650 (Abcam); ab79414 (Abcam); MAB69201 (R&D Systems); HM57 (Bio- |
| | Rad) |
| CD79b | Any CD79b antibody described in WO 2014/011521; ab130422 (Abcam); |
| | ab134147 (Abcam); polatuzumab (Genentech) |

In certain embodiments, the TAA is selected from BCMA and CD20. In some embodiments, the TAA is BCMA. “BCMA” refers to B-cell maturation antigen. BCMA (also known as TNFRSF17, BCM or CD269) is a member of the tumor necrosis receptor (TNFR) family and is predominantly expressed on terminally differentiated B cells, e.g., memory B cells and plasma cells. Its ligands include B-cell activating factor (BAFF) and a proliferation-inducing ligand (APRIL). The protein BCMA is encoded by the gene TNFRSF17. Exemplary BCMA sequences are available at the Uniprot database under accession number Q02223.

In certain aspects, a Type 2 TBM comprises an ABM3 that specifically binds to BCMA, for example, an anti-BCMA antibody or an antigen-binding domain thereof. The anti-BCMA antibody or antigen-binding domain thereof can comprise, for example, CDR, VH, VL, or scFV sequences set forth in Tables 11A-11G of WO 2019/195535, the contents of which are incorporated herein by reference in their entireties.

### 7.2.8. Nucleic Acids and Host Cells

The CD19 binding molecules described herein can be encoded by a single nucleic acid or, alternatively, encoded by a plurality of (e.g., two, three, four or more) nucleic acids.

A single nucleic acid can encode a CD19 binding molecule that comprises a single polypeptide chain, a CD19 binding molecule that comprises two or more polypeptide chains, or a portion of a CD19 binding molecule that comprises more than two polypeptide chains (for example, a single nucleic acid can encode two polypeptide chains of a CD19 binding molecule comprising three, four or more polypeptide chains, or three polypeptide chains of a CD19 binding molecule comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements, and separated by internal ribosome entry site (IRES) sequences allowing for translation into separate polypeptides.

In some embodiments, a CD19 binding molecule comprising two or more polypeptide chains is encoded by two or more nucleic acids. The number of nucleic acids encoding a CD19 binding molecule can be equal to or less than the number of polypeptide chains in the CD19 binding molecule (for example, when more than one polypeptide chains are encoded by a single nucleic acid).

The nucleic acids can be DNA or RNA (e.g., mRNA).

Host cells can be genetically engineered to comprise one or more nucleic acids encoding a CD19 binding molecule. In one embodiment, the host cells are genetically engineered by using an expression cassette. The phrase “expression cassette,” refers to nucleotide sequences, which are capable of affecting expression of a gene in hosts compatible with such sequences. Such cassettes can include a promoter, an open reading frame with or without introns, and a termination signal. Additional factors necessary or helpful in effecting expression can also be used, such as, for example, an inducible promoter. The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 7.3. CAR Molecules

In some aspects, the anti-CD19 agent used in the methods and combinations of the disclosure is a population of cells that expresses a chimeric antigen receptor (CAR) molecule that binds CD19. As used herein, the term “CAR molecule” encompasses both CARs that are contiguous polypeptides and CARs that are non-contiguous polypeptides. Typically the treatment with a CAR is by way of administration of a population of cells that express the CD19 CAR molecule.

In certain aspects, the CAR molecule comprises a chimeric fusion protein comprising an extracellular antigen binding domain, a transmembrane domain and an intracellular signaling domain comprising a functional signaling domain derived from a stimulatory molecule, and optionally includes one or more functional signaling domains derived from one or more costimulatory molecules. For example, the CAR molecule can comprise a chimeric fusion protein comprising an extracellular antigen binding domain, a transmembrane domain and an intracellular signaling domain comprising two functional signaling domains derived from one or more costimulatory molecule(s) and a functional signaling domain derived from a stimulatory molecule. Extracellular antigen binding domains, transmembrane domains and intracellular signaling domains are described in Sections 7.3.1, 7.3.2 and 7.3.3, respectively, and exemplary CAR sequences are set forth in Section 7.3.4.

In some instances, the transmembrane domain can be attached to the extracellular region of the CAR, e.g., the antigen binding domain of the CAR, via a hinge. Exemplary hinge sequences are described in Section 7.3.2.

The CAR can also comprise an optional leader sequence at the amino-terminus (N-ter) of the CAR fusion protein, which when present is typically located at the N-terminus of the extracellular antigen binding domain. The leader sequence can be cleaved from the antigen binding domain (e.g., a scFv) during cellular processing and localization of the CAR to the cellular membrane and accordingly a CAR composition administered the subject may lack the leader sequence. Leader sequences useful in the CAR molecules of the disclosure are described in Section 7.3.1.

Further aspects of the CAR molecules useful in the methods and combinations of the disclosure are described below.

### 7.3.1. CD19 Binding Domain and Optional Leader

The portion of a CAR comprising an antibody or antibody fragment thereof may exist in a variety of forms where the antigen binding domain is expressed as part of a contiguous polypeptide chain including, for example, a single domain antibody fragment (sdAb), a single chain antibody (scFv), a humanized antibody, or bispecific antibody (Harlow et al., 1999, In: Using Antibodies: A Laboratory Manual, Cold Spring Harbor Laboratory Press, NY; Harlow et al., 1989, In: Antibodies: A Laboratory Manual, Cold Spring Harbor, New York; Houston et al., 1988, Proc. Natl. Acad. Sci. USA 85:5879-5883; Bird et al., 1988, Science 242:423-426). In one aspect, the antigen binding domain of a CAR comprises an antibody fragment. In a further aspect, the CAR comprises an antibody fragment that comprises a scFv.

In one aspect, the portion of the CAR comprising the antigen binding domain comprises an antigen binding domain that targets CD19. In one aspect, the antigen binding domain targets human CD19. In one aspect, the antigen binding domain of the CAR has the same or a similar binding specificity as, or includes, the FMC63 scFv fragment described in Nicholson et al., 1997, Mol. Immun. 34 (16-17): 1157-1165. In one aspect, the portion of the CAR comprising the antigen binding domain comprises an antigen binding domain that targets a B-cell antigen, e.g., a human B-cell antigen. A CD19 antibody molecule can be, e.g., an antibody molecule (e.g., a humanized anti-CD19 antibody molecule) described in WO2014/153270, which is incorporated herein by reference in its entirety. WO2014/153270 also describes methods of assaying the binding and efficacy of various CART constructs.

In some embodiments, the CD19 CAR comprises an antigen binding domain derived from (e.g., comprises an amino acid sequence of) an anti-CD19 antibody (e.g., an anti-CD19 mono- or bispecific antibody) or a fragment or conjugate thereof. In one embodiment, the anti-CD19 antibody is a humanized antigen binding domain as described in WO2014/153270 (e.g., Table 1 of WO2014/153270) incorporated herein by reference, or a conjugate thereof. Other exemplary anti-CD19 antibodies or fragments or conjugates thereof, include but are not limited to, a bispecific T cell engager that targets CD19 (e.g., blinatumomab), SAR3419 (Sanofi), MEDI-551 (Medlmmune LLC), Combotox, DT2219ARL (Masonic Cancer Center), MOR-208 (also called XmAb-5574; MorphoSys), XmAb-5871 (Xencor), MDX-1342 (Bristol-Myers Squibb), SGN-CD19A (Seattle Genetics), and AFM11 (Affimed Therapeutics). See, e.g., Hammer. MAbs. 4.5(2012): 571-77. Blinatomomab is a bispecific antibody comprised of two scFvs-one that binds to CD19 and one that binds to CD3. Blinatomomab directs T cells to attack cancer cells. See, e.g., Hammer et al.; Clinical Trial Identifier No. NCT00274742 and NCT01209286. MEDI-551 is a humanized anti-CD19 antibody with a Fc engineered to have enhanced antibody-dependent cell-mediated cytotoxicity (ADCC). See, e.g., Hammer et al.; and Clinical Trial Identifier No. NCT01957579. Combotox is a mixture of immunotoxins that bind to CD19 and CD22. The immunotoxins are made up of scFv antibody fragments fused to a deglycosylated ricin A chain. See, e.g., Hammer et al.; and Herrera et al. J. Pediatr. Hematol. Oncol. 31.12(2009):936-41; Schindler et al. Br. J. Haematol. 154.4(2011):471-6. DT2219ARL is a bispecific immunotoxin targeting CD19 and CD22, comprising two scFvs and a truncated diphtheria toxin. See, e.g., Hammer et al.; and Clinical Trial Identifier No. NCT00889408. SGN-CD19A is an antibody-drug conjugate (ADC) comprised of an anti-CD19 humanized monoclonal antibody linked to a synthetic cytotoxic cell-killing agent, monomethyl auristatin F (MMAF). See, e.g., Hammer et al.; and Clinical Trial Identifier Nos. NCT01786096 and NCT01786135. SAR3419 is an anti-CD19 antibody-drug conjugate (ADC) comprising an anti-CD19 humanized monoclonal antibody conjugated to a maytansine derivative via a cleavable linker. See, e.g., Younes et al. J. Clin. Oncol. 30.2(2012): 2776-82; Hammer et al.; Clinical Trial Identifier No. NCT00549185; and Blanc et al. Clin Cancer Res. 2011; 17:6448-58. XmAb-5871 is an Fc-engineered, humanized anti-CD19 antibody. See, e.g., Hammer et al. MDX-1342 is a human Fc-engineered anti-CD19 antibody with enhanced ADCC. See, e.g., Hammer et al. In some embodiments, the antibody molecule is a bispecific anti-CD19 and anti-CD3 molecule. For instance, AFM11 is a bispecific antibody that targets CD19 and CD3. See, e.g., Hammer et al.; and Clinical Trial Identifier No. NCT02106091.

In certain embodiments, a CAR molecule used in the methods and combinations of the disclosure is monospecific and has specificity only for CD19, whether or not the CD19 binding domain is derived from a mono- or multispecific antibody.

In one embodiment, an antigen binding domain against CD19 is an antigen binding portion, e.g., CDRs, of an antigen binding domain described in a Table herein, e.g., in Table 1 or in this Section 7.3, including its subparts. In one embodiment, a CD19 antigen binding domain can be from any CD19 CAR, e.g., LG-740; U.S. Pat. Nos. 8,399,645; 7,446,190; Xu et al., 2013, Leuk Lymphoma. 54(2):255-260(2012); Cruz et al., 2013, Blood 122(17):2965-2973; Brentjens et al., 2011, Blood, 118(18):4817-4828; Kochenderfer et al., 2010, Blood 116(20):4099-102; Kochenderfer et al., 2013, Blood 122 (25):4129-39; and 16th Annu Meet Am Soc Gen Cell Ther (ASGCT) (May 15-18, Salt Lake City) 2013, Abst 10, each of which is herein incorporated by reference in its entirety.

In one aspect, the anti-CD19 protein binding portion of the CAR is a scFv antibody fragment. In one aspect such antibody fragments are functional in that they retain the equivalent binding affinity, e.g., they bind the same antigen with comparable affinity, as the IgG antibody from which it is derived. In one aspect such antibody fragments are functional in that they provide a biological response that can include, but is not limited to, activation of an immune response, inhibition of signal-transduction origination from its target antigen, inhibition of kinase activity, and the like, as will be understood by a skilled artisan. In one aspect, the anti-CD19 antigen binding domain of the CAR is a scFv antibody fragment that is humanized compared to the murine sequence of the scFv from which it is derived. In one aspect, the parental murine scFv sequence is the CAR19 construct provided in PCT publication WO2012/079000 and provided herein as SEQ ID NO:149. In one embodiment, the anti-CD19 binding domain is a scFv described in WO2012/079000 and provided in SEQ ID NO: 149, or a sequence at least 95%, e.g., 95-99%, identical thereto. In an embodiment, the anti-CD19 binding domain is part of a CAR construct provided in POT publication WO2012/079000 and provided herein as SEQ ID NO: 148, or a sequence at least 95%, e.g., 95%-99%, identical thereto. In an embodiment, the anti-CD19 binding domain comprises at least one (e.g., 2, 3, 4, 5, or 6) CDRs selected from Table 14 and/or Table 15.

TABLE 14
Heavy Chain Variable Domain CDRs

| | | | SEQ | | SEQ | | SEQ |
|---|---|---|---|---|---|---|---|
| | | | ID | | ID | | ID |
| Molecule | FW | CDR-H1 | NO | CDR-H2 | NO | CDR-H3 | NO |
| murine_CART19 | | GVSLPDYGVS | 113 | VIWGSETTYYNSALKS | 114 | HYYYGGSYAMDY | 118 |
| humanized_CART19 a | VH4 | GVSLPDYGVS | 113 | VIWGSETTYYSSSLKS | 115 | HYYYGGSYAMDY | 118 |
| humanized_CART19 b | VH4 | GVSLPDYGVS | 113 | VIWGSETTYYQSSLKS | 116 | HYYYGGSYAMDY | 118 |
| humanized_CART19 c | VH4 | GVSLPDYGVS | 113 | VIWGSETTYYNSSLKS | 117 | HYYYGGSYAMDY | 118 |

TABLE 15
Light Chain Variable Domain CDRs

| | | | SEQ | | SEQ | | SEQ |
|---|---|---|---|---|---|---|---|
| | | | ID | | ID | | ID |
| Candidate | FW | CDR-L1 | NO | CDR-L2 | NO | CDR-L3 | NO |
| murine_CART19 | | RASQDISKYLN | 119 | HTSRLHS | 120 | QQGNTLPYT | 121 |
| humanized_CART19 a | VK3 | RASQDISKYLN | 119 | HTSRLHS | 120 | QQGNTLPYT | 121 |
| humanized_CART19 b | VK3 | RASQDISKYLN | 119 | HTSRLHS | 120 | QQGNTLPYT | 121 |
| humanized_CART19 c | VK3 | RASQDISKYLN | 119 | HTSRLHS | 120 | QQGNTLPYT | 121 |

In one aspect, the CAR comprises the polypeptide sequence provided as SEQ ID NO:12 in POT publication WO2012/079000, and provided herein as SEQ ID NO:149, wherein the scFv domain is substituted by one or more sequences selected from SEQ ID NOS: 96-107. In one aspect, the scFv domains of SEQ ID NOS:96-107 are humanized variants of the scFv domain of SEQ ID NO: 149, which is an scFv fragment of murine origin that specifically binds to human 0019. Humanization of this mouse scFv may be desired for the clinical setting, where the mouse-specific residues may induce a human-anti-mouse antigen (HAMA) response in subjects who receive CART19 treatment, e.g., treatment with T cells transduced with the CAR19 construct.

In one embodiment, the CD19 CAR comprises an amino acid sequence provided as SEQ ID NO:12 in PCT publication WO2012/079000. In embodiment, the amino acid sequence is:

(SEQ ID NO: 148)
MALPVTALLLPLALLLHAARPdiqmtqttsslsaslgdrvtiscrasqdiskylnwyqqkpdgtvklliyhtsrlhsg
vpsrfsgsgsgtdysltisnleqediatyfcqqgntlpytfgggtkleitggggsggggsggggsevklqesgpglvapsqsl
svtctvsgvslpdygvswirqpprkglewlgviwgsettyynsalksrltiikdnsksqvflkmnslqtddtaiyycakhyyyg
gsyamdywgqgtsvtvsstttpaprpptpaptiasqplslrpeacrpaaggavhtrgldfacdiyiwaplagtcgvlllslvitly
ckrgrkkllyifkqpfmrpvqttqeedgcscrfpeeeeggcelrvkfsrsadapaykqgqnqlynelnlgrreeydvldkrrgr
dpemggkprrknpqeglynelqkdkmaeayseigmkgerrrgkghdglyqglstatkdtydalhmqalppr,

or a sequence substantially homologous thereto.

In another embodiment, the amino acid sequence is

(SEQ ID NO: 213)
diqmtqttsslsaslgdrvtiscrasqdiskylnwyqqkpdgtvklliyhtsrlhsgvpsrfsgsgsgtdysltisnleqediatyf
cqqgntlpytfgggtkleitggggsggggsggggsevklqesgpglvapsqslsvtctvsgvslpdygvswirqpprkgle
wlgviwgsettyynsalksrltiikdnsksqvflkmnslqtddtaiyycakhyyyggsyamdywgqgtsvtvsstttpaprpp
tpaptiasqplslrpeacrpaaggavhtrgldfacdiyiwaplagtcgvlllslvitlyckrgrkkllyifkqpfmrpvqttqeedgc
scrfpeeeeggcelrvkfsrsadapaykqgqnqlynelnlgrreeydvldkrrgrdpemggkprrknpqeglynelqkdk
maeayseigmkgerrrgkghdglyqglstatkdtydalhmqalppr

or a sequence substantially homologous thereto.

In one aspect, the humanized CAR19 comprises the scFv portion provided in SEQ ID NO:96. In one aspect, the humanized CAR19 comprises the scFv portion provided in SEQ ID NO:97. In one aspect, the humanized CAR19 comprises the scFv portion provided in SEQ ID NO:98. In one aspect, the humanized CAR19 comprises the scFv portion provided in SEQ ID NO:99. In one aspect, the humanized CAR19 comprises the scFv portion provided in SEQ ID NO:100. In one aspect, the humanized CAR19 comprises the scFv portion provided in SEQ ID NO:101. In one aspect, the humanized CAR19 comprises the scFv portion provided in SEQ ID NO:102. In one aspect, the humanized CAR19 comprises the scFv portion provided in SEQ ID NO:103. In one aspect, the humanized CAR19 comprises the scFv portion provided in SEQ ID NO:104. In one aspect, the humanized CAR19 comprises the scFv portion provided in SEQ ID NO:105. In one aspect, the humanized CAR19 comprises the scFv portion provided in SEQ ID NO:106. In one aspect, the humanized CAR19 comprises the scFv portion provided in SEQ ID NO:107.

In one aspect, the CARs of the disclosure combine an antigen binding domain of a specific antibody with an intracellular signaling molecule. For example, in some aspects, the intracellular signaling molecule includes, but is not limited to, CD3-zeta chain, 4-1BB and CD28 signaling modules and combinations thereof. In one aspect, the CD19 CAR comprises a CAR selected from the sequence provided in one or more of SEQ ID NOS: 122-133. In one aspect, the CD19 CAR comprises the sequence provided in SEQ ID NO:122. In one aspect, the CD19 CAR comprises the sequence provided in SEQ ID NO:123. In one aspect, the CD19 CAR comprises the sequence provided in SEQ ID NO:124. In one aspect, the CD19 CAR comprises the sequence provided in SEQ ID NO:125. In one aspect, the CD19 CAR comprises the sequence provided in SEQ ID NO:126. In one aspect, the CD19 CAR comprises the sequence provided in SEQ ID NO:127. In one aspect, the CD19 CAR comprises the sequence provided in SEQ ID NO:128. In one aspect, the CD19 CAR comprises the sequence provided in SEQ ID NO:129. In one aspect, the CD19 CAR comprises the sequence provided in SEQ ID NO:130. In one aspect, the CD19 CAR comprises the sequence provided in SEQ ID NO:131. In one aspect, the CD19 CAR comprises the sequence provided in SEQ ID NO:132. In one aspect, the CD19 CAR comprises the sequence provided in SEQ ID NO:133.

In some embodiments, the CAR molecule is a CD19 CAR molecule described herein, e.g., a humanized CAR molecule described herein, e.g., a humanized CD19 CAR molecule of Table 16 or having CDRs as set out in Table 14 and Table 15.

In some embodiments, the CAR molecule is a CD19 CAR molecule described herein, e.g., a murine CAR molecule described herein, e.g., a murine CD19 CAR molecule of Table 17 or having CDRs as set out in Table 14 and Table 15.

In some embodiments, the CAR molecule comprises one, two, and/or three CDRs from the heavy chain variable region and/or one, two, and/or three CDRs from the light chain variable region of the murine or humanized CD19 CAR of Table 14 and Table 15.

In one embodiment, the antigen binding domain comprises one, two three (e.g., all three) heavy chain CDRs, CDR-H1, CDR-H2 and CDR-H3, from an antibody listed above, and/or one, two, three (e.g., all three) light chain CDRs, CDR-L1, CDR-L2 and CDR-L3, from an antibody listed above. In one embodiment, the antigen binding domain comprises a heavy chain variable region and/or a variable light chain region of an antibody listed or described above.

In an embodiment, the antigen binding domain comprises a humanized antibody or an antibody fragment. In one embodiment, the humanized anti-CD19 binding domain comprises one or more (e.g., all three) light chain complementary determining region 1 (CDR-L1), light chain complementary determining region 2 (CDR-L2), and light chain complementary determining region 3 (CDR-L3) of a murine or humanized anti-CD19 binding domain described herein, and/or one or more (e.g., all three) heavy chain complementary determining region 1 (CDR-H1), heavy chain complementary determining region 2 (CDR-H2), and heavy chain complementary determining region 3 (CDR-H3) of a murine or humanized anti-CD19 binding domain described herein, e.g., a humanized anti-CD19 binding domain comprising one or more, e.g., all three, light chain CDRs and one or more, e.g., all three, heavy chain CDRs.

In one embodiment, an antigen binding domain comprises one, two three (e.g., all three) heavy chain CDRs, CDR-H1, CDR-H2 and CDR-H3, from an antibody listed herein, e.g., in Table 14, Table 16, or Table 17 and/or one, two, three (e.g., all three) light chain CDRs, CDR-L1, CDR-L2 and CDR-L3, from an antibody listed herein, e.g., in Table 15, Table 16, or Table 17. In one embodiment, the antigen binding domain comprises a heavy chain variable region and/or a variable light chain region of an antibody listed or described above.

In an embodiment, the CD19 binding domain (e.g., an scFv) comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 16 or Table 17, or a sequence with at least 95% (e.g., 95-99%) identity with an amino acid sequence of Table 16 or Table 17; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 16 or Table 17, or a sequence with at least 95% (e.g., 95-99%) identity to an amino acid sequence of Table 16 or Table 17.

In some embodiments, the CD19 binding domain comprises one or more CDRs (e.g., one each of a CDR-H1, CDR-H2, CDR-H3, CDR-L1, CDR-L2, and CDR-L3) of Table 16 or Table 17, or CDRs having one, two, three, four, five, or six modifications (e.g., substitutions) of one or more of the CDRs.

Exemplary anti-CD19 antibody molecules (including antibodies or fragments or conjugates thereof) can include a scFv, CDRs, or VH and VL chains described in any one of Table 14, Table 15, Table 16, or Table 17. In an embodiment, the CD19-binding antibody molecule comprises: a light chain variable region comprising an amino acid sequence having at least one, two or three modifications (e.g., substitutions) but not more than 30, 20 or 10 modifications (e.g., substitutions) of an amino acid sequence of a light chain variable region provided in Table 16 or Table 17, or a sequence with at least 95% (e.g., 95-99%) identity with an amino acid sequence of Table 16 or Table 17; and/or a heavy chain variable region comprising an amino acid sequence having at least one, two or three modifications (e.g., substitutions) but not more than 30, 20 or 10 modifications (e.g., substitutions) of an amino acid sequence of a heavy chain variable region provided in Table 16 or Table 17, or a sequence with at least 95% (e.g., 95-99%) identity to an amino acid sequence of Table 16 or Table 17. In some embodiments, the CD19-binding antibody molecule comprises one or more CDRs (e.g., one each of a CDR-H1, CDR-H2, CDR-H3, CDR-L1, CDR-L2, and CDR-L3) of Table 14 or Table 15, or CDRs having one, two, three, four, five, or six modifications (e.g., substitutions) of one or more of the CDRs.

In some embodiments, the humanized anti-CD19 binding domain comprises a CDR-H1, a CDR-H2, and a CDR-H3 of any heavy chain binding domain amino acid sequences listed in Table 16 or Table 17. In some embodiments, the antigen binding domain further comprises a CDR-L1, a CDR-L2, and a CDR-L3. In some embodiments, the antigen binding domain comprises a CDR-L1, a CDR-L2, and a CDR-L3 of any light chain binding domain amino acid sequences listed in Table 16 or Table 17.

In some embodiments, the antigen binding domain comprises one, two or all of CDR-11, CDR-L2, and CDR-L3 of any light chain binding domain amino acid sequences listed in Table 3 or Table 17, and one, two or all of CDR-H1, CDR-H2, and CDR-H3 of any heavy chain binding domain amino acid sequences listed in Table 17.

In some embodiments, the CDRs are defined according to the Kabat numbering scheme, the Chothia numbering scheme, or a combination thereof.

The sequences of humanized CDR sequences of the scFv domains are shown in Table 14 for the heavy chain variable domains and in Table 15 for the light chain variable domains. “ID” stands for the respective SEQ ID NO for each CDR.

In some embodiments, the CD19 binding domain comprises a Kabat CDR-H1 having a sequence of DYGVS (SEQ ID NO:214), an CDR-H2 of Table 14, an CDR-H3 of Table 14, an CDR-L1 of Table 15, an CDR-L2 of Table 15, and an CDR-L3 of Table 15.

In one embodiment, the humanized anti-CD19 binding domain comprises a sequence selected from a group consisting of SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106, and SEQ ID NO:107, or a sequence with 95-99% identity thereof. In one embodiment, the nucleic acid sequence encoding the humanized anti-CD19 binding domain comprises a sequence selected from a group consisting of SEQ ID NO:151, SEQ ID NO:152, SEQ ID NO:153, SEQ ID NO:154, SEQ ID NO:155, SEQ ID NO:156, SEQ ID NO:157, SEQ ID NO:158, SEQ ID NO:159, SEQ ID NO:160 and SEQ ID NO:161, or a sequence with 95-99% identity thereof.

In one embodiment, the humanized anti-CD19 binding domain is a scFv, and a light chain variable region comprising an amino acid sequence described herein, e.g., in Table 16 or Table 17, is attached to a heavy chain variable region comprising an amino acid sequence described herein, e.g., in Table 16 or Table 17, via a linker, e.g., a linker described herein. In one embodiment, the humanized anti-CD19 binding domain includes a (Gly₄-Ser)n linker, wherein n is 1, 2, 3, 4, 5, or 6, e.g., 3 or 4 (SEQ ID NO:144). The light chain variable region and heavy chain variable region of a scFv can be, e.g., in any of the following orientations: light chain variable region-linker-heavy chain variable region or heavy chain variable region-linker-light chain variable region.

In one aspect, the antigen binding domain portion comprises one or more sequence selected from SEQ ID NOS:96-107. In one aspect the humanized CAR is selected from one or more sequence selected from SEQ ID NOS: 122-133. In some aspects, a non-human antibody is humanized, where specific sequences or regions of the antibody are modified to increase similarity to an antibody naturally produced in a human or fragment thereof.

In one embodiment, the anti-CD19 binding domain is a scFv, and a light chain variable region comprising an amino acid sequence described herein, e.g., in Table 16 or Table 17, is attached to a heavy chain variable region comprising an amino acid sequence described herein, e.g., in Table 16 or Table 17, via a linker, e.g., a linker described herein. In one embodiment, the antigen binding domain includes a (Gly₄-Ser)n linker, wherein n is 1, 2, 3, 4, 5, or 6, e.g., 3 or 4 (SEQ ID NO:144). The light chain variable region and heavy chain variable region of a scFv can be, e.g., in any of the following orientations: light chain variable region-linker-heavy chain variable region or heavy chain variable region-linker-light chain variable region.

In some embodiments, the CAR molecule comprises a CD19 binding domain, a transmembrane domain, and an intracellular signaling domain comprising a stimulatory domain, and wherein said CD19 binding domain comprises one or more of (e.g., all three of) light chain complementary determining region 1 (CDR-L1), light chain complementary determining region 2 (CDR-L2), and light chain complementary determining region 3 (CDR-L3) of any CD19 light chain binding domain amino acid sequence listed in Table 16 or Table 17, and one or more of (e.g., all three of) heavy chain complementary determining region 1 (CDR-H1), heavy chain complementary determining region 2 (CDR-H2), and heavy chain complementary determining region 3 (CDR-H3) of any CD19 heavy chain binding domain amino acid sequence listed in Table 16 or Table 17.

In some embodiments, a CD19 CAR comprises light chain variable region listed in Table 16 or Table 17 and any heavy chain variable region listed Table 16 or Table 17.

In some embodiments, the CAR molecule comprises a CD19 binding domain which comprises a sequence selected from a group consisting of SEQ ID NO:96, SEQ ID NO:97, SEQ ID NO:98, SEQ ID NO:99, SEQ ID NO:100, SEQ ID NO:101, SEQ ID NO:102, SEQ ID NO:103, SEQ ID NO:104, SEQ ID NO:105, SEQ ID NO:106 and SEQ ID NO:107, or a sequence with 95-99% identity thereof. In some embodiments, the CD19 CAR comprises a polypeptide of SEQ ID NO:148.

In one embodiment, the CAR molecule comprises an anti-CD19 binding domain comprising one or more (e.g., all three) light chain complementary determining region 1 (CDR-L1), light chain complementary determining region 2 (CDR-L2), and light chain complementary determining region 3 (CDR-L3) of an anti-CD19 binding domain described herein, and one or more (e.g., all three) heavy chain complementary determining region 1 (CDR-H1), heavy chain complementary determining region 2 (CDR-H2), and heavy chain complementary determining region 3 (CDR-H3) of an anti-CD19 binding domain described herein, e.g., an anti-CD19 binding domain comprising one or more, e.g., all three, light chain CDRs and one or more, e.g., all three, heavy chain CDRs. In one embodiment, the anti-CD19 binding domain comprises one or more (e.g., all three) heavy chain complementary determining region 1 (CDR-H1), heavy chain complementary determining region 2 (CDR-H2), and heavy chain complementary determining region 3 (CDR-H3) of an anti-CD19 binding domain described herein, e.g., the anti-CD19 binding domain has two variable heavy chain regions, each comprising a CDR-H1, a CDR-H2 and a CDR-H3 described herein.

In one aspect, the anti-CD19 binding domain is characterized by particular functional features or properties of an antibody or antibody fragment. For example, in one aspect, the portion of a CAR molecule that comprises an antigen binding domain specifically binds human CD19. In one aspect, the disclosure relates to an antigen binding domain comprising an antibody or antibody fragment, wherein the antibody binding domain specifically binds to a CD19 protein or fragment thereof, wherein the antibody or antibody fragment comprises a variable light chain and/or a variable heavy chain that includes an amino acid sequence of SEQ ID NO:96-107 or SEQ ID NO:149. In one aspect, the antigen binding domain comprises an amino acid sequence of an scFv selected from SEQ ID NOs: 96-107 or SEQ ID NO:149. In certain aspects, the scFv is contiguous with and in the same reading frame as a leader sequence.

### 7.3.2. Transmembrane Domain and Optional Hinge

With respect to the transmembrane domain, in various embodiments, a CAR can be designed to comprise a transmembrane domain that is attached to the extracellular domain of the CAR. A transmembrane domain can include one or more additional amino acids adjacent to the transmembrane region, e.g., one or more amino acid associated with the extracellular region of the protein from which the transmembrane was derived (e.g., 1, 2, 3, 4, 5, 6, 7, 8, 9, 10 up to 15 amino acids of the extracellular region) and/or one or more additional amino acids associated with the intracellular region of the protein from which the transmembrane protein is derived (e.g., 1, 2, 3, 4, 5, 6, 7, 8, 9, 10 up to 15 amino acids of the intracellular region). In one aspect, the transmembrane domain is one that is associated with one of the other domains of the CAR. In one embodiment, the transmembrane domain may be from the same protein that the signaling domain, costimulatory domain or the hinge domain is derived from. In another aspect, the transmembrane domain is not derived from the same protein that any other domain of the CAR is derived from. In some instances, the transmembrane domain can be selected or modified by amino acid substitution to avoid binding of such domains to the transmembrane domains of the same or different surface membrane proteins, e.g., to minimize interactions with other members of the receptor complex. In one aspect, the transmembrane domain is capable of homodimerization with another CAR on the cell surface of a CAR-expressing cell. In a different aspect the amino acid sequence of the transmembrane domain may be modified or substituted so as to minimize interactions with the binding domains of the native binding partner present in the same CAR-expressing cell.

The transmembrane domain may be derived either from a natural or from a recombinant source. Where the source is natural, the domain may be derived from any membrane-bound or transmembrane protein. In one aspect the transmembrane domain is capable of signaling to the intracellular domain(s) whenever the CAR has bound to a target. A transmembrane domain of particular use in this disclosure may include at least the transmembrane region(s) of e.g., the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. In some embodiments, a transmembrane domain may include at least the transmembrane region(s) of, e.g., KIRDS2, OX40, CD2, CD27, LFA-1 (CD11a, CD18), ICOS (CD278), 4-1BB (CD137), GITR, CD40, BAFFR, HVEM (LIGHTR), SLAMF7, NKp80 (KLRF1), NKp44, NKp30, NKp46, CD160, CD19, IL2R beta, IL2R gamma, IL7R α, ITGA1, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, TNFR2, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), SLAMF6 (NTB-A, Ly108), SLAM (SLAMF1, CD150, IPO-3), BLAME (SLAMF8), SELPLG (CD162), LTBR, PAG/Cbp, NKG2D, NKG2C, or CD19.

In some instances, the transmembrane domain can be attached to the extracellular region of the CAR, e.g., the antigen binding domain of the CAR, via a hinge, e.g., a hinge from a human protein. For example, in one embodiment, the hinge can be a human Ig (immunoglobulin) hinge, e.g., an IgG4 hinge, an IgD hinge, a GS linker (e.g., a GS linker described herein), a KIR2DS2 hinge, or a CD8a hinge.

In one aspect, the transmembrane domain may be recombinant, in which case it will comprise predominantly hydrophobic residues such as leucine and valine. In one aspect a triplet of phenylalanine, tryptophan and valine can be found at each end of a recombinant transmembrane domain.

Optionally, a short oligo- or polypeptide linker, between 2 and 10 amino acids in length may form the linkage between the transmembrane domain and the cytoplasmic region of the CAR. A glycine-serine doublet provides a particularly suitable linker. For example, in one aspect, the linker comprises the amino acid sequence of GGGGSGGGGS (SEQ ID NO:140). In some embodiments, the linker is encoded by a nucleotide sequence of GGTGGCGGAGGTTCTGGAGGTGGAGGTTCC (SEQ ID NO:141).

In one aspect, the hinge or spacer comprises a KIR2DS2 hinge.

### 7.3.3. Intracellular Signaling Domain

The cytoplasmic domain or region of the CAR includes an intracellular signaling domain. An intracellular signaling domain is generally responsible for activation of at least one of the normal effector functions of the immune cell in which the CAR has been introduced.

Examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any recombinant sequence that has the same functional capability.

It is known that signals generated through the TCR alone are insufficient for full activation of the T cell and that a secondary and/or costimulatory signal is also required. Thus, T cell activation can be said to be mediated by two distinct classes of cytoplasmic signaling sequences: those that initiate antigen-dependent primary activation through the TCR (primary intracellular signaling domains) and those that act in an antigen-independent manner to provide a secondary or costimulatory signal (secondary cytoplasmic domain, e.g., a costimulatory domain).

A primary signaling domain regulates primary activation of the TCR complex either in a stimulatory way, or in an inhibitory way. Primary intracellular signaling domains that act in a stimulatory manner may contain signaling motifs which are known as immunoreceptor tyrosine-based activation motifs or ITAMs.

Examples of ITAM containing primary intracellular signaling domains that are of particular use in the disclosure include those of CD3-zeta, common FcR gamma (FCER1G), Fc gamma RIIa, FcR beta (Fc Epsilon R1b), CD3 gamma, CD3 delta, CD3 epsilon, CD79a, CD79b, CD278 (also known as “ICOS”), FcεRI, DAP10, DAP12, and CD66d. In one embodiment, a CAR of the disclosure comprises an intracellular signaling domain, e.g., a primary signaling domain of CD3-zeta.

In certain embodiments, the stimulatory molecule is the zeta chain associated with the T cell receptor complex. In one aspect, the cytoplasmic signaling domain further comprises one or more functional signaling domains derived from at least one costimulatory molecule as defined below.

In further embodiments, the costimulatory molecule is chosen from the costimulatory molecules described herein, e.g., 4-1BB (i.e., CD137), CD27 and/or CD28.

Accordingly, a CAR molecule that can be used in the methods and combinations of the disclosure can comprise at least one intracellular domain selected from the group of a CD137 (4-1BB) signaling domain, a CD28 signaling domain, a CD3-zeta signaling domain, and any combination thereof and/or at least one intracellular signaling domain is from one or more co-stimulatory molecule(s), which are optionally other than a CD137 (4-1BB) or CD28.

In one embodiment, a primary signaling domain comprises a modified ITAM domain, e.g., a mutated ITAM domain which has altered (e.g., increased or decreased) activity as compared to the native ITAM domain. In one embodiment, a primary signaling domain comprises a modified ITAM-containing primary intracellular signaling domain, e.g., an optimized and/or truncated ITAM-containing primary intracellular signaling domain. In an embodiment, a primary signaling domain comprises one, two, three, four or more ITAM motifs.

Further examples of molecules containing a primary intracellular signaling domain that are of particular use in the disclosure include those of DAP10, DAP12, and CD32. In an embodiment, the intracellular signaling domain (also referred to as the cytoplasmic domain) can comprise a primary intracellular signaling domain. Exemplary primary intracellular signaling domains include those derived from the molecules responsible for primary stimulation, or antigen dependent simulation. In an embodiment, the intracellular signaling domain can comprise a costimulatory intracellular domain. Exemplary costimulatory intracellular signaling domains include those derived from molecules responsible for costimulatory signals, or antigen independent stimulation. For example, in the case of a CART, a primary intracellular signaling domain can comprise a cytoplasmic sequence of a T cell receptor, and a costimulatory intracellular signaling domain can comprise cytoplasmic sequence from co-receptor or costimulatory molecule.

Primary Intracellular Signaling Domain: A primary intracellular signaling domain can comprise a signaling motif which is known as an immunoreceptor tyrosine-based activation motif or ITAM. Examples of ITAM containing primary cytoplasmic signaling sequences include, but are not limited to, those derived from CD3-zeta, FcR gamma, common FcR gamma (FCER1G), Fc gamma RIIa, FcR beta (Fc Epsilon R1b), CD3 gamma, CD3 delta, CD3 epsilon, CD22, CD79a, CD79b, CD278 (“ICOS”), FcεRI, CD66d, CD32, DAP10 and DAP12.

Costimulatory Intracellular Signaling Domain: The intracellular signaling domain of the CAR can comprise the CD3-zeta signaling domain by itself or it can be combined with any other desired intracellular signaling domain(s) useful in the context of a CAR of the disclosure. For example, the intracellular signaling domain of the CAR can comprise a CD3-zeta chain portion and a costimulatory signaling domain. The costimulatory signaling domain refers to a portion of the CAR comprising the intracellular domain of a costimulatory molecule.

A costimulatory molecule can be a cell surface molecule other than an antigen receptor or its ligands that is required for an efficient response of lymphocytes to an antigen. Examples of such molecules include CD27, CD28, 4-1BB (CD137), OX40, CD30, CD40, PD1, ICOS, lymphocyte function-associated antigen-1 (LFA-1), CD2, CD7, LIGHT, NKG2C, B7-H3, and a ligand that specifically binds with CD83, and the like. For example, CD27 costimulation has been demonstrated to enhance expansion, effector function, and survival of human CART cells in vitro and augments human T cell persistence and antitumor activity in vivo (Song et al. Blood. 2012; 119(3):696-706). Further examples of such costimulatory molecules include MHC class I molecule, TNF receptor proteins, Immunoglobulin-like proteins, cytokine receptors, integrins, signaling lymphocytic activation molecules (SLAM proteins), activating NK cell receptors, BTLA, a Toll ligand receptor, OX40, CD2, CD7, CD27, CD28, CD30, CD40, CDS, ICAM-1, LFA-1 (CD11a/CD18), 4-1BB (CD137), B7-H3, CDS, ICAM-1, ICOS (CD278), GITR, BAFFR, LIGHT, HVEM (LIGHTR), KIRDS2, SLAMF7, NKp80 (KLRF1), NKp44, NKp30, NKp46, CD19, CD4, CD8alpha, CD8beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, LFA-1, ITGB7, NKG2D, NKG2C, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Ly108), SLAM (SLAMF1, CD150, IPO-3), BLAME (SLAMF8), SELPLG (CD162), LTBR, LAT, GADS, SLP-76, PAG/Cbp, CD19a, and a ligand that specifically binds with CD83.

In one embodiment, the intracellular domain is designed to comprise the signaling domain of CD3-zeta and the signaling domain of CD28. In one aspect, the intracellular domain is designed to comprise the signaling domain of CD3-zeta and the signaling domain of ICOS.

The intracellular signaling sequences within the cytoplasmic portion of the CAR of the disclosure may be linked to each other in a random or specified order. Optionally, a short oligo- or polypeptide linker, for example, between 2 and 10 amino acids (e.g., 2, 3, 4, 5, 6, 7, 8, 9, or 10 amino acids) in length may form the linkage between intracellular signaling sequence. In one embodiment, a glycine-serine doublet can be used as a suitable linker. In one embodiment, a single amino acid, e.g., an alanine, a glycine, can be used as a suitable linker.

In one aspect, the intracellular signaling domain is designed to comprise two or more, e.g., 2, 3, 4, 5, or more, costimulatory signaling domains. In an embodiment, the two or more, e.g., 2, 3, 4, 5, or more, costimulatory signaling domains, are separated by a linker molecule, e.g., a linker molecule described herein. In one embodiment, the intracellular signaling domain comprises two costimulatory signaling domains. In some embodiments, the linker molecule is a glycine residue. In some embodiments, the linker is an alanine residue.

In one aspect, the intracellular signaling domain is designed to comprise the signaling domain of CD3-zeta and the signaling domain of CD28. In one aspect, the intracellular signaling domain is designed to comprise the signaling domain of CD3-zeta and the signaling domain of 4-1BB. In one aspect, the signaling domain of 4-1BB is a signaling domain of SEQ ID NO:1156. In one aspect, the signaling domain of CD3-zeta is a signaling domain of SEQ ID NO:1160 or SEQ ID NO:1162. In certain aspects, the CAR-T comprises a CAR molecule having the sequence of SEQ ID NO:97 or SEQ ID NO:149.

In one aspect, the intracellular signaling domain is designed to comprise the signaling domain of CD3-zeta and the signaling domain of CD27.

A costimulatory intracellular signaling domain refers to the intracellular portion of a costimulatory molecule. The intracellular signaling domain can comprise the entire intracellular portion, or the entire native intracellular signaling domain, of the molecule from which it is derived, or a functional fragment or derivative thereof.

### 7.3.4. Exemplary CAR Molecules

Provided herein are the sequence of exemplary CAR molecules that can be used in the methods and combinations of the disclosure as well as their encoding nucleic acid sequences. Typically a CAR molecule useful in the methods and combinations of the disclosure is encoded by CAR construct that encodes an optional leader sequence, an extracellular antigen binding domain, a hinge, a transmembrane domain, and an intracellular stimulatory domain. In some embodiments, the CAR constructs further encodes an intracellular costimulatory domain, such that the expressed CAR molecule comprises an optional leader sequence, an extracellular antigen binding domain, a hinge, a transmembrane domain, an intracellular costimulatory domain and an intracellular stimulatory domain.

Exemplary CAR component sequences are shown in Table C.

TABLE C
Sequences of various components of CAR

| SEQ ID | | |
|---|---|---|
| NO | Description | Sequence |
| 1144 | EF-1α | CGTGAGGCTCCGGTGCCCGTCAGTGGGCAGAGCGCACATCGCCCACA |
| | promoter (na) | GTCCCCGAGAAGTTGGGGGGAGGGGTCGGCAATTGAACCGGTGCCTA |
| | | GAGAAGGTGGCGCGGGGTAAACTGGGAAAGTGATGTCGTGTACTGGCT |
| | | CCGCCTTTTTCCCGAGGGTGGGGGAGAACCGTATATAAGTGCAGTAGT |
| | | CGCCGTGAACGTTCTTTTTCGCAACGGGTTTGCCGCCAGAACACAGGTA |
| | | AGTGCCGTGTGTGGTTCCCGCGGGCCTGGCCTCTTTACGGGTTATGGC |
| | | CCTTGCGTGCCTTGAATTACTTCCACCTGGCTGCAGTACGTGATTCTTG |
| | | ATCCCGAGCTTCGGGTTGGAAGTGGGTGGGAGAGTTCGAGGCCTTGCG |
| | | CTTAAGGAGCCCCTTCGCCTCGTGCTTGAGTTGAGGCCTGGCCTGGGC |
| | | GCTGGGGCCGCCGCGTGCGAATCTGGTGGCACCTTCGCGCCTGTCTC |
| | | GCTGCTTTCGATAAGTCTCTAGCCATTTAAAATTTTTGATGACCTGCTGC |
| | | GACGCTTTTTTTCTGGCAAGATAGTCTTGTAAATGCGGGCCAAGATCTG |
| | | CACACTGGTATTTCGGTTTTTGGGGCCGCGGGCGGCGACGGGGCCCG |
| | | TGCGTCCCAGCGCACATGTTCGGCGAGGCGGGGCCTGCGAGCGCGGC |
| | | CACCGAGAATCGGACGGGGGTAGTCTCAAGCTGGCCGGCCTGCTCTG |
| | | GTGCCTGGCCTCGCGCCGCCGTGTATCGCCCCGCCCTGGGCGGCAAG |
| | | GCTGGCCCGGTCGGCACCAGTTGCGTGAGCGGAAAGATGGCCGCTTC |
| | | CCGGCCCTGCTGCAGGGAGCTCAAAATGGAGGACGCGGCGCTCGGGA |
| | | GAGCGGGCGGGTGAGTCACCCACACAAAGGAAAAGGGCCTTTCCGTCC |
| | | TCAGCCGTCGCTTCATGTGACTCCACGGAGTACCGGGCGCCGTCCAGG |
| | | CACCTCGATTAGTTCTCGAGCTTTTGGAGTACGTCGTCTTTAGGTTGGG |
| | | GGGAGGGGTTTTATGCGATGGAGTTTCCCCACACTGAGTGGGTGGAGA |
| | | CTGAAGTTAGGCCAGCTTGGCACTTGATGTAATTCTCCTTGGAATTTGC |
| | | CCTTTTTGAGTTTGGATCTTGGTTCATTCTCAAGCCTCAGACAGTGGTTC |
| | | AAAGTTTTTTTCTTCCATTTCAGGTGTCGTGA |
| 1145 | Leader (aa) | MALPVTALLLPLALLLHAARP |
| 1146 | Leader (na) | ATGGCCCTGCCTGTGACAGCCCTGCTGCTGCCTCTGGCTCTGCTGCTG |
| | | CATGCCGCTAGACCC |
| 1147 | Leader (na) | ATGGCCCTCCCTGTCACCGCCCTGCTGCTTCCGCTGGCTCTTCTGCTC |
| | | CACGCCGCTCGGCCC |
| 1148 | CD8 hinge | TTTPAPRPPTPAPTIASQPLSLRPEACRPAAGGAVHTRGLDFACD |
| 1149 | CD8 hinge (na) | ACCACGACGCCAGCGCCGCGACCACCAACACCGGCGCCCACCATCGC |
| | | GTCGCAGCCCCTGTCCCTGCGCCCAGAGGCGTGCCGGCCAGCGGCGG |
| | | GGGGCGCAGTGCACACGAGGGGGCTGGACTTCGCCTGTGAT |
| 1150 | Ig4 hinge (aa) | ESKYGPPCPPCPAPEFLGGPSVFLFPPKPKDTLMISRTPEVTCVVVDVSQE |
| | | DPEVQFNWYVDGVEVHNAKTKPREEQFNSTYRVVSVLTVLHQDWLNGKE |
| | | YKCKVSNKGLPSSIEKTISKAKGQPREPQVYTLPPSQEEMTKNQVSLTCLV |
| | | KGFYPSDIAVEWESNGQPENNYKTTPPVLDSDGSFFLYSRLTVDKSRWQE |
| | | GNVFSCSVMHEALHNHYTQKSLSLSLGKM |
| 1151 | Ig4 hinge (na) | GAGAGCAAGTACGGCCCTCCCTGCCCCCCTTGCCCTGCCCCCGAGTTC |
| | | CTGGGCGGACCCAGCGTGTTCCTGTTCCCCCCCAAGCCCAAGGACACC |
| | | CTGATGATCAGCCGGACCCCCGAGGTGACCTGTGTGGTGGTGGACGTG |
| | | TCCCAGGAGGACCCCGAGGTCCAGTTCAACTGGTACGTGGACGGCGTG |
| | | GAGGTGCACAACGCCAAGACCAAGCCCCGGGAGGAGCAGTTCAATAG |
| | | CACCTACCGGGTGGTGTCCGTGCTGACCGTGCTGCACCAGGACTGGCT |
| | | GAACGGCAAGGAATACAAGTGTAAGGTGTCCAACAAGGGCCTGCCCAG |
| | | CAGCATCGAGAAAACCATCAGCAAGGCCAAGGGCCAGCCTCGGGAGC |
| | | CCCAGGTGTACACCCTGCCCCCTAGCCAAGAGGAGATGACCAAGAACC |
| | | AGGTGTCCCTGACCTGCCTGGTGAAGGGCTTCTACCCCAGCGACATCG |
| | | CCGTGGAGTGGGAGAGCAACGGCCAGCCCGAGAACAACTACAAGACC |
| | | ACCCCCCCTGTGCTGGACAGCGACGGCAGCTTCTTCCTGTACAGCCGG |
| | | CTGACCGTGGACAAGAGCCGGTGGCAGGAGGGCAACGTCTTTAGCTGC |
| | | TCCGTGATGCACGAGGCCCTGCACAACCACTACACCCAGAAGAGCCTG |
| | | AGCCTGTCCCTGGGCAAGATG |
| 1152 | IgD hinge (aa) | RWPESPKAQASSVPTAQPQAEGSLAKATTAPATTRNTGRGGEEKKKEKEK |
| | | EEQEERETKTPECPSHTQPLGVYLLTPAVQDLWLRDKATFTCFVVGSDLK |
| | | DAHLTWEVAGKVPTGGVEEGLLERHSNGSQSQHSRLTLPRSLWNAGTSV |
| | | TCTLNHPSLPPQRLMALREPAAQAPVKLSLNLLASSDPPEAASWLLCEVSG |
| | | FSPPNILLMWLEDQREVNTSGFAPARPPPQPGSTTFWAWSVLRVPAPPSP |
| | | QPATYTCVVSHEDSRTLLNASRSLEVSYVTDH |
| 1153 | IgD hinge (na) | AGGTGGCCCGAAAGTCCCAAGGCCCAGGCATCTAGTGTTCCTACTGCA |
| | | CAGCCCCAGGCAGAAGGCAGCCTAGCCAAAGCTACTACTGCACCTGCC |
| | | ACTACGCGCAATACTGGCCGTGGCGGGGAGGAGAAGAAAAAGGAGAA |
| | | AGAGAAAGAAGAACAGGAAGAGAGGGAGACCAAGACCCCTGAATGTCC |
| | | ATCCCATACCCAGCCGCTGGGCGTCTATCTCTTGACTCCCGCAGTACAG |
| | | GACTTGTGGCTTAGAGATAAGGCCACCTTTACATGTTTCGTCGTGGGCT |
| | | CTGACCTGAAGGATGCCCATTTGACTTGGGAGGTTGCCGGAAAGGTAC |
| | | CCACAGGGGGGGTTGAGGAAGGGTTGCTGGAGCGCCATTCCAATGGC |
| | | TCTCAGAGCCAGCACTCAAGACTCACCCTTCCGAGATCCCTGTGGAAC |
| | | GCCGGGACCTCTGTCACATGTACTCTAAATCATCCTAGCCTGCCCCCAC |
| | | AGCGTCTGATGGCCCTTAGAGAGCCAGCCGCCCAGGCACCAGTTAAGC |
| | | TTAGCCTGAATCTGCTCGCCAGTAGTGATCCCCCAGAGGCCGCCAGCT |
| | | GGCTCTTATGCGAAGTGTCCGGCTTTAGCCCGCCCAACATCTTGCTCAT |
| | | GTGGCTGGAGGACCAGCGAGAAGTGAACACCAGCGGCTTCGCTCCAG |
| | | CCCGGCCCCCACCCCAGCCGGGTTCTACCACATTCTGGGCCTGGAGTG |
| | | TCTTAAGGGTCCCAGCACCACCTAGCCCCCAGCCAGCCACATACACCT |
| | | GTGTTGTGTCCCATGAAGATAGCAGGACCCTGCTAAATGCTTCTAGGAG |
| | | TCTGGAGGTTTCCTACGTGACTGACCATT |
| 1154 | CD8 | IYIWAPLAGTCGVLLLSLVITLYC |
| | Transmembrane | |
| | (aa) | |
| 1155 | CD8 | ATCTACATCTGGGCGCCCTTGGCCGGGACTTGTGGGGTCCTTCTCCTG |
| | Transmembrane | TCACTGGTTATCACCCTTTACTGC |
| | (na) | |
| 1156 | 4-1BB | KRGRKKLLYIFKQPFMRPVQTTQEEDGCSCRFPEEEEGGCEL |
| | intracellular | |
| | domain (aa) | |
| 1157 | 4-1BB | AAACGGGGCAGAAAGAAACTCCTGTATATATTCAAACAACCATTTATGA |
| | intracellular | GACCAGTACAAACTACTCAAGAGGAAGATGGCTGTAGCTGCCGATTTCC |
| | domain (na) | AGAAGAAGAAGAAGGAGGATGTGAACTG |
| 1158 | CD27 (aa) | QRRKYRSNKGESPVEPAEPCRYSCPREEEGSTIPIQEDYRKPEPACSP |
| 1159 | CD27 (na) | AGGAGTAAGAGGAGCAGGCTCCTGCACAGTGACTACATGAACATGACT |
| | | CCCCGCCGCCCCGGGCCCACCCGCAAGCATTACCAGCCCTATGCCCC |
| | | ACCACGCGACTTCGCAGCCTATCGCTCC |
| 1160 | CD3-zeta (aa) | RVKFSRSADAPAYKQGQNQLYNELNLGRREEYDVLDKRRGRDPEMGGKP |
| | (Q/K mutant) | RRKNPQEGLYNELQKDKMAEAYSEIGMKGERRRGKGHDGLYQGLSTATK |
| | | DTYDALHMQALPPR |
| 1161 | CD3-zeta (na) | AGAGTGAAGTTCAGCAGGAGCGCAGACGCCCCCGCGTACAAGCAGGG |
| | (Q/K mutant) | CCAGAACCAGCTCTATAACGAGCTCAATCTAGGACGAAGAGAGGAGTA |
| | | CGATGTTTTGGACAAGAGACGTGGCCGGGACCCTGAGATGGGGGGAAA |
| | | GCCGAGAAGGAAGAACCCTCAGGAAGGCCTGTACAATGAACTGCAGAA |
| | | AGATAAGATGGCGGAGGCCTACAGTGAGATTGGGATGAAAGGCGAGCG |
| | | CCGGAGGGGCAAGGGGCACGATGGCCTTTACCAGGGTCTCAGTACAG |
| | | CCACCAAGGACACCTACGACGCCCTTCACATGCAGGCCCTGCCCCCTC |
| | | GC |
| 1162 | CD3-zeta (aa) | RVKFSRSADAPAYQQGQNQLYNELNLGRREEYDVLDKRRGRDPEMGGKP |
| | (NCBI | RRKNPQEGLYNELQKDKMAEAYSEIGMKGERRRGKGHDGLYQGLSTATK |
| | Reference | DTYDALHMQALPPR |
| | Sequence | |
| | NM_000734.3) | |
| 1163 | CD3-zeta (na) | AGAGTGAAGTTCAGCAGGAGCGCAGACGCCCCCGCGTACCAGCAGGG |
| | (NCBI | CCAGAACCAGCTCTATAACGAGCTCAATCTAGGACGAAGAGAGGAGTA |
| | Reference | CGATGTTTTGGACAAGAGACGTGGCCGGGACCCTGAGATGGGGGGAAA |
| | Sequence | GCCGAGAAGGAAGAACCCTCAGGAAGGCCTGTACAATGAACTGCAGAA |
| | NM_000734.3) | AGATAAGATGGCGGAGGCCTACAGTGAGATTGGGATGAAAGGCGAGCG |
| | | CCGGAGGGGCAAGGGGCACGATGGCCTTTACCAGGGTCTCAGTACAG |
| | | CCACCAAGGACACCTACGACGCCCTTCACATGCAGGCCCTGCCCCCTC |
| | | GC |
| 1164 | CD28 | RSKRSRLLHSDYMNMTPRRPGPTRKHYQPYAPPRDFAAYRS |
| | Intracellular | |
| | domain (amino | |
| | acid sequence) | |
| 1165 | CD28 | AGGAGTAAGAGGAGCAGGCTCCTGCACAGTGACTACATGAACATGACT |
| | Intracellular | CCCCGCCGCCCCGGGCCCACCCGCAAGCATTACCAGCCCTATGCCCC |
| | domain | ACCACGCGACTTCGCAGCCTATCGCTCC |
| | (nucleotide | |
| | sequence) | |
| 1166 | ICOS | TKKKYSSSVHDPNGEYMFMRAVNTAKKSRLTDVTL |
| | Intracellular | |
| | domain (amino | |
| | acid sequence) | |
| 1167 | ICOS | ACAAAAAAGAAGTATTCATCCAGTGTGCACGACCCTAACGGTGAATACA |
| | Intracellular | TGTTCATGAGAGCAGTGAACACAGCCAAAAAATCCAGACTCACAGATGT |
| | domain | GACCCTA |
| | (nucleotide | |
| | sequence) | |
| 1168 | GS hinge/linker | GGGGSGGGGS |
| 1169 | GS hinge/linker | GGTGGCGGAGGTTCTGGAGGTGGAGGTTCC |
| | (na) | |
| 1170 | GS hinge/linker | GGTGGCGGAGGTTCTGGAGGTGGGGGTTCC |
| | (na) | |
| 1171 | linker | GGGGS |
| 1172 | linker | (Gly-Gly-Gly-Gly-Ser)n, where n = 1-6, for example, |
| | | GGGGSGGGGS GGGGSGGGGS GGGGSGGGGS |
| 1173 | linker | GGGGSGGGGSGGGGSGGGGS |
| 1174 | linker | GGGGSGGGGSGGGGS |
| 1175 | linker | GGGS |
| 1176 | linker | (Gly-Gly-Gly-Ser)n where n is a positive integer equal to |
| | | or greater than 1 |
| 1177 | linker | (Gly-Gly-Gly-Ser)n, where n = 1-10, for example, GGGSGGGSGG |
| | | GSGGGSGGGS GGGSGGGSGG GSGGGSGGGS |
| 1178 | linker | GSTSGSGKPGSGEGSTKG |

In certain aspects, the CAR molecule comprises a CD19 CAR molecule described in US-2015-0283178-A1, for example a CD19 CAR comprising an amino acid, or encoded by a nucleotide sequence, described in US-2015-0283178-A1, incorporated herein by reference. In some embodiments, the CAR molecule comprises the sequence of SEQ ID NO: 149 or SEQ ID NO:97. In certain aspects, the CAR molecule comprises the sequence of SEQ ID NO: 1162 or SEQ ID NO: 1160.

In further aspects, the CAR molecule comprise an amino acid sequence, or are encoded by nucleic acid constructs, described in International Application WO2014/153270, certain sequences of which are reproduced herein.

The sequences of the humanized scFv fragments (SEQ ID NOS: 96-107) are provided below in Table 16.

TABLE 16
Humanized CD19 CAR Constructs

| | | |
|---|---|---|
| | SEQ ID | |
| Name | NO | Sequence |

CAR 1

| | | |
|---|---|---|
| CAR1 | 96 | EIVMTQSPATLSLSPGERATLSCRASQDISKYLNWYQQKPGQAPRL |
| scFv | | LIYHTSRLHSGIPARFSGSGSGTDYTLTISSLQPEDFAVYFCQQGNT |
| domain | | LPYTFGQGTKLEIKGGGGSGGGGSGGGGSQVQLQESGPGLVKPS |
| | | ETLSLTCTVSGVSLPDYGVSWIRQPPGKGLEWIGVIWGSETTYYSS |
| | | SLKSRVTISKDNSKNQVSLKLSSVTAADTAVYYCAKHYYYGGSYAM |
| | | DYWGQGTLVTVSS |
| 103101 | 151 | atggccctccctgtcaccgccctgctgcttccgctggctcttctgctccacgccgctcggcccgaa |
| CAR1 | | attgtgatgacccagtcacccgccactcttagcctttcacccggtgagcgcgcaaccctgtcttgc |
| Soluble | | agagcctcccaagacatctcaaaataccttaattggtatcaacagaagcccggacaggctcct |
| scFv-nt | | cgccttctgatctaccacaccagccggctccattctggaatccctgccaggttcagcggtagcgg |
| | | atctgggaccgactacaccctcactatcagctcactgcagccagaggacttcgctgtctatttctg |
| | | tcagcaagggaacaccctgccctacacctttggacagggcaccaagctcgagattaaaggtg |
| | | gaggtggcagcggaggaggtgggtccggcggtggaggaagccaggtccaactccaagaa |
| | | agcggaccgggtcttgtgaagccatcagaaactctttcactgacttgtactgtgagcggagtgtct |
| | | ctccccgattacggggtgtcttggatcagacagccaccggggaagggtctggaatggattgga |
| | | gtgatttggggctctgagactacttactactcttcatccctcaagtcacgcgtcaccatctcaaagg |
| | | acaactctaagaatcaggtgtcactgaaactgtcatctgtgaccgcagccgacaccgccgtgta |
| | | ctattgcgctaagcattactattatggcgggagctacgcaatggattactggggacagggtactc |
| | | tggtcaccgtgtccagccaccaccatcatcaccatcaccat |
| 103101 | 163 | MALPVTALLLPLALLLHAARPeivmtqspatlslspgeratlscrasqdiskylnwy |
| CAR1 | | qqkpgqaprlliyhtsrlhsgiparfsgsgsgtdytltisslqpedfavyfcqqgntlpytfgqgtklei |
| Soluble | | kggggsggggsggggsqvqlqesgpglvkpsetlsltctvsgvslpdygvswirqppgkglew |
| scFv-aa | | igviwgsettyyssslksrvtiskdnsknqvslklssvtaadtavyycakhyyyggsyamdywg |
| | | qgtlvtvsshhhhhhhh |
| 104875 | 175 | atggccctccctgtcaccgccctgctgcttccgctggctcttctgctccacgccgctcggcccgaa |
| CAR 1- | | attgtgatgacccagtcacccgccactcttagcctttcacccggtgagcgcgcaaccctgtcttgc |
| Full-nt | | agagcctcccaagacatctcaaaataccttaattggtatcaacagaagcccggacaggctcct |
| | | cgccttctgatctaccacaccagccggctccattctggaatccctgccaggttcagcggtagcgg |
| | | atctgggaccgactacaccctcactatcagctcactgcagccagaggacttcgctgtctatttctg |
| | | tcagcaagggaacaccctgccctacacctttggacagggcaccaagctcgagattaaaggtg |
| | | gaggtggcagcggaggaggtgggtccggcggtggaggaagccaggtccaactccaagaa |
| | | agcggaccgggtcttgtgaagccatcagaaactctttcactgacttgtactgtgagcggagtgtct |
| | | ctccccgattacggggtgtcttggatcagacagccaccggggaagggtctggaatggattgga |
| | | gtgatttggggctctgagactacttactactcttcatccctcaagtcacgcgtcaccatctcaaagg |
| | | acaactctaagaatcaggtgtcactgaaactgtcatctgtgaccgcagccgacaccgccgtgta |
| | | ctattgcgctaagcattactattatggcgggagctacgcaatggattactggggacagggtactc |
| | | tggtcaccgtgtccagcaccactaccccagcaccgaggccacccaccccggctcctaccatc |
| | | gcctcccagcctctgtccctgcgtccggaggcatgtagacccgcagctggtggggccgtgcat |
| | | acccggggtcttgacttcgcctgcgatatctacatttgggcccctctggctggtacttgcggggtcc |
| | | tgctgctttcactcgtgatcactctttactgtaagcgcggtcggaagaagctgctgtacatctttaag |
| | | caacccttcatgaggcctgtgcagactactcaagaggaggacggctgttcatgccggttcccag |
| | | aggaggaggaaggcggctgcgaactgcgcgtgaaattcagccgcagcgcagatgctccag |
| | | cctacaagcaggggcagaaccagctctacaacgaactcaatcttggtcggagagaggagta |
| | | cgacgtgctggacaagcggagaggacgggacccagaaatgggcgggaagccgcgcaga |
| | | aagaatccccaagagggcctgtacaacgagctccaaaaggataagatggcagaagcctata |
| | | gcgagattggtatgaaaggggaacgcagaagaggcaaaggccacgacggactgtaccag |
| | | ggactcagcaccgccaccaaggacacctatgacgctcttcacatgcaggccctgccgcctcg |
| | | g |
| 104875 | 122 | MALPVTALLLPLALLLHAARPeivmtqspatlslspgeratlscrasqdiskylnwy |
| CAR 1- | | qqkpgqaprlliyhtsrlhsgiparfsgsgsgtdytltisslqpedfavyfcqqgntlpytfgqgtkl |
| Full-aa | | eikggggsggggsggggsqvqlqesgpglvkpsetlsltctvsgvslpdygvswirqppgkgl |
| | | ewigviwgsettyyssslksrvtiskdnsknqvslklssvtaadtavyycakhyyyggsya |
| | | mdywgqgtlvtvsstttpaprpptpaptiasqplslrpeacrpaaggavhtrgldfacdiyiwapl |
| | | agtcgvlllslvitlyckrgrkkllyifkqpfmrpvqttqeedgcscrfpeeeeggcelrvkfsrsada |
| | | paykqgqnqlynelnlgrreeydvldkrrgrdpemggkprrknpqeglynelqkdkmaeays |
| | | eigmkgerrrgkghdglyqglstatkdtydalhmqalppr |

CAR 2

| | | |
|---|---|---|
| CAR2 | 97 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsqvqlqesgpgl |
| domain | | vkpsetlsltctvsgvslpdygvswirqppgkglewigviwgsettyyqsslksrvtiskdnsknq |
| | | vslklssvtaadtavyycakhyyyggsyamdywgqgtlvtvss |
| 103102 | 152 | atggccctccctgtcaccgccctgctgcttccgctggctcttctgctccacgccgctcggcccgaa |
| CAR2- | | attgtgatgacccagtcacccgccactcttagcctttcacccggtgagcgcgcaaccctgtcttgc |
| Soluble | | agagcctcccaagacatctcaaaataccttaattggtatcaacagaagcccggacaggctcct |
| scFv-nt | | cgccttctgatctaccacaccagccggctccattctggaatccctgccaggttcagcggtagcgg |
| | | atctgggaccgactacaccctcactatcagctcactgcagccagaggacttcgctgtctatttctg |
| | | tcagcaagggaacaccctgccctacacctttggacagggcaccaagctcgagattaaaggtg |
| | | gaggtggcagcggaggaggtgggtccggcggtggaggaagccaggtccaactccaagaa |
| | | agcggaccgggtcttgtgaagccatcagaaactctttcactgacttgtactgtgagcggagtgtct |
| | | ctccccgattacggggtgtcttggatcagacagccaccggggaagggtctggaatggattgga |
| | | gtgatttggggctctgagactacttactaccaatcatccctcaagtcacgcgtcaccatctcaaag |
| | | gacaactctaagaatcaggtgtcactgaaactgtcatctgtgaccgcagccgacaccgccgtgt |
| | | actattgcgctaagcattactattatggcgggagctacgcaatggattactggggacagggtact |
| | | ctggtcaccgtgtccagccaccaccatcatcaccatcaccat |
| 103102 | 164 | MALPVTALLLPLALLLHAARPeivmtqspatlslspgeratlscrasqdiskylnwy |
| CAR2- | | qqkpgqaprlliyhtsrlhsgiparfsgsgsgtdytltisslqpedfavyfcqqgntlpytfgqgtklei |
| Soluble | | kggggggggsggggsqvqlqesgpglvkpsetlsltctvsgvslpdygvswirqppgkglew |
| scFv-aa | | igviwgsettyyqsslksrvtiskdnsknqvslklssvtaadtavyycakhyyyggsyamdywg |
| | | qgtlvtvsshhhhhhhh |
| 104876 | 176 | atggccctccctgtcaccgccctgctgcttccgctggctcttctgctccacgccgctcggcccgaa |
| CAR 2- | | attgtgatgacccagtcacccgccactcttagcctttcacccggtgagcgcgcaaccctgtcttgc |
| Full-nt | | agagcctcccaagacatctcaaaataccttaattggtatcaacagaagcccggacaggctcct |
| (also | | cgccttctgatctaccacaccagccggctccattctggaatccctgccaggttcagcggtagcgg |
| referred to | | atctgggaccgactacaccctcactatcagctcactgcagccagaggacttcgctgtctatttctg |
| herein as | | tcagcaagggaacaccctgccctacacctttggacagggcaccaagctcgagattaaaggtg |
| CTL119 | | gaggtggcagcggaggaggtgggtccggcggtggaggaagccaggtccaactccaagaa |
| nucleotide | | agcggaccgggtcttgtgaagccatcagaaactctttcactgacttgtactgtgagcggagtgtct |
| sequence) | | ctccccgattacggggtgtcttggatcagacagccaccggggaagggtctggaatggattgga |
| | | gtgatttggggctctgagactacttactaccaatcatccctcaagtcacgcgtcaccatctcaaag |
| | | gacaactctaagaatcaggtgtcactgaaactgtcatctgtgaccgcagccgacaccgccgtgt |
| | | actattgcgctaagcattactattatggcgggagctacgcaatggattactggggacagggtact |
| | | ctggtcaccgtgtccagcaccactaccccagcaccgaggccacccaccccggctcctaccat |
| | | cgcctcccagcctctgtccctgcgtccggaggcatgtagacccgcagctggtggggccgtgcat |
| | | acccggggtcttgacttcgcctgcgatatctacatttgggcccctctggctggtacttgcggggtcc |
| | | tgctgctttcactcgtgatcactctttactgtaagcgcggtcggaagaagctgctgtacatctttaag |
| | | caacccttcatgaggcctgtgcagactactcaagaggaggacggctgttcatgccggttcccag |
| | | aggaggaggaaggcggctgcgaactgcgcgtgaaattcagccgcagcgcagatgctccag |
| | | cctacaagcaggggcagaaccagctctacaacgaactcaatcttggtcggagagaggagta |
| | | cgacgtgctggacaagcggagaggacgggacccagaaatgggcgggaagccgcgcaga |
| | | aagaatccccaagagggcctgtacaacgagctccaaaaggataagatggcagaagcctata |
| | | gcgagattggtatgaaaggggaacgcagaagaggcaaaggccacgacggactgtaccag |
| | | ggactcagcaccgccaccaaggacacctatgacgctcttcacatgcaggccctgccgcctcg |
| | | g |
| 104876 | 123 | MALPVTALLLPLALLLHAARPeivmtqspatlslspgeratlscrasqdiskylnwy |
| CAR 2- | | qqkpgqaprlliyhtsrlhsgiparfsgsgsgtdytltisslqpedfavyfcqqgntlpytfgqgtkl |
| Full-aa | | eikggggsggggsggggsqvqlqesgpglvkpsetlsltctvsgvslpdygvswirqppgkgl |
| (also | | ewigviwgsettyyqsslksrvtiskdnsknqvslklssvtaadtavyycakhyyyggsya |
| referred to | | mdywgqgtlvtvsstttpaprpptpaptiasqplslrpeacrpaaggavhtrgldfacdiyiwapl |
| herein as | | agtcgvlllslvitlyckrgrkkllyifkqpfmrpvqttqeedgcscrfpeeeeggcelrvkfsrsada |
| CTL119 | | paykqgqnqlynelnlgrreeydvldkrrgrdpemggkprrknpqeglynelqkdkmaeays |
| amino acid | | eigmkgerrrgkghdglyqglstatkdtydalhmqalppr |
| sequence) | | |

CAR 3

| | | |
|---|---|---|
| CAR3 | 98 | qvqlqesgpglvkpsetlsltctvsgvslpdygvswirqppgkglewigviwgsettyyssslksr |
| scFv | | vtiskdnsknqvslklssvtaadtavyycakhyyyggsyamdywgqgtlvtvssggggsggg |
| domain | | gsggggseivmtqspatlslspgeratlscrasqdiskylnwyqqkpgqaprlliyhtsrlhsgip |
| | | arfsgsgsgtdytltisslqpedfavyfcqqgntlpytfgqgtkleik |
| 103104 | 153 | atggctctgcccgtgaccgcactcctcctgccactggctctgctgcttcacgccgctcgcccaca |
| CAR 3- | | agtccagcttcaagaatcagggcctggtctggtgaagccatctgagactctgtccctcacttgca |
| Soluble | | ccgtgagcggagtgtccctcccagactacggagtgagctggattagacagcctcccggaaag |
| scFv-nt | | ggactggagtggatcggagtgatttggggtagcgaaaccacttactattcatcttccctgaagtc |
| | | acgggtcaccatttcaaaggataactcaaagaatcaagtgagcctcaagctctcatcagtcac |
| | | cgccgctgacaccgccgtgtattactgtgccaagcattactactatggagggtcctacgccatgg |
| | | actactggggccagggaactctggtcactgtgtcatctggtggaggaggtagcggaggaggc |
| | | gggagcggtggaggtggctccgaaatcgtgatgacccagagccctgcaaccctgtccctttctc |
| | | ccggggaacgggctaccctttcttgtcgggcatcacaagatatctcaaaatacctcaattggtat |
| | | caacagaagccgggacaggcccctaggcttcttatctaccacacctctcgcctgcatagcggg |
| | | attcccgcacgctttagcgggtctggaagcgggaccgactacactctgaccatctcatctctcca |
| | | gcccgaggacttcgccgtctacttctgccagcagggtaacaccctgccgtacaccttcggccag |
| | | ggcaccaagcttgagatcaaacatcaccaccatcatcaccatcac |
| 103104 | 165 | MALPVTALLLPLALLLHAARPqvqlqesgpglvkpsetlsltctvsgvslpdygvs |
| CAR 3- | | wirqppgkglewigviwgsettyyssslksrvtiskdnsknqvslklssvtaadtavyycakhyy |
| Soluble | | yggsyamdywgqgtlvtvssggggsggggsggggseivmtqspatlslspgeratlscrasq |
| scFv-aa | | diskylnwyqqkpgqaprlliyhtsrlhsgiparfsgsgsgtdytltisslqpedfavyfcqqgntlp |
| | | ytfgqgtkleikhhhhhhhh |
| 104877 | 177 | atggctctgcccgtgaccgcactcctcctgccactggctctgctgcttcacgccgctcgcccaca |
| CAR 3- | | agtccagcttcaagaatcagggcctggtctggtgaagccatctgagactctgtccctcacttgca |
| Full-nt | | ccgtgagcggagtgtccctcccagactacggagtgagctggattagacagcctcccggaaag |
| | | ggactggagtggatcggagtgatttggggtagcgaaaccacttactattcatcttccctgaagtc |
| | | acgggtcaccatttcaaaggataactcaaagaatcaagtgagcctcaagctctcatcagtcac |
| | | cgccgctgacaccgccgtgtattactgtgccaagcattactactatggagggtcctacgccatgg |
| | | actactggggccagggaactctggtcactgtgtcatctggtggaggaggtagcggaggaggc |
| | | gggagcggtggaggtggctccgaaatcgtgatgacccagagccctgcaaccctgtccctttctc |
| | | ccggggaacgggctaccctttcttgtcgggcatcacaagatatctcaaaatacctcaattggtat |
| | | caacagaagccgggacaggcccctaggcttcttatctaccacacctctcgcctgcatagcggg |
| | | attcccgcacgctttagcgggtctggaagcgggaccgactacactctgaccatctcatctctcca |
| | | gcccgaggacttcgccgtctacttctgccagcagggtaacaccctgccgtacaccttcggccag |
| | | ggcaccaagcttgagatcaaaaccactactcccgctccaaggccacccacccctgccccgac |
| | | catcgcctctcagccgctttccctgcgtccggaggcatgtagacccgcagctggtggggccgtg |
| | | catacccggggtcttgacttcgcctgcgatatctacatttgggcccctctggctggtacttgcgggg |
| | | tcctgctgctttcactcgtgatcactctttactgtaagcgcggtcggaagaagctgctgtacatcttt |
| | | aagcaacccttcatgaggcctgtgcagactactcaagaggaggacggctgttcatgccggttc |
| | | ccagaggaggaggaaggcggctgcgaactgcgcgtgaaattcagccgcagcgcagatgct |
| | | ccagcctacaagcaggggcagaaccagctctacaacgaactcaatcttggtcggagagagg |
| | | agtacgacgtgctggacaagcggagaggacgggacccagaaatggggggaagccgcgc |
| | | agaaagaatccccaagagggcctgtacaacgagctccaaaaggataagatggcagaagcc |
| | | tatagcgagattggtatgaaaggggaacgcagaagaggcaaaggccacgacggactgtac |
| | | cagggactcagcaccgccaccaaggacacctatgacgctcttcacatgcaggccctgccgcc |
| | | tcgg |
| 104877 | 124 | MALPVTALLLPLALLLHAARPqvqlqesgpglvkpsetlsltctvsgvslpdygvswi |
| CAR 3- | | rqppgkglewigviwgsettyyssslksrvtiskdnsknqvslklssvtaadtavyycakhyy |
| Full-aa | | yggsyamdywgqgtlvtvssggggsggggsggggseivmtqspatlslspgeratlscras |
| | | qdiskylnwyqqkpgqaprlliyhtsrlhsgiparfsgsgsgtdytltisslqpedfavyfcqqg |
| | | ntlpytfgqgtkleiktttpaprpptpaptiasqplsIrpeacrpaaggavhtrgldfacdiyiwapl |
| | | agtcgvlllslvitlyckrgrkkllyifkqpfmrpvqttqeedgcscrfpeeeeggcelrvkfsrsada |
| | | paykqgqnqlynelnlgrreeydvldkrrgrdpemggkprrknpqeglynelqkdkmaeays |
| | | eigmkgerrrgkghdglyqglstatkdtydalhmqalppr |

CAR 4

| | | |
|---|---|---|
| CAR4 | 99 | qvqlqesgpglvkpsetlsltctvsgvslpdygvswirqppgkglewigviwgsettyyqsslksr |
| scFv | | vtiskdnsknqvslklssvtaadtavyycakhyyyggsyamdywgqgtlvtvssggggsggg |
| domain | | gsggggseivmtqspatlslspgeratlscrasqdiskyInwyqqkpgqaprlliyhtsrlhsgip |
| | | arfsgsgsgtdytltisslqpedfavyfcqqgntlpytfgqgtkleik |
| 103106 | 154 | atggctctgcccgtgaccgcactcctcctgccactggctctgctgcttcacgccgctcgcccaca |
| CAR4- | | agtccagcttcaagaatcagggcctggtctggtgaagccatctgagactctgtccctcacttgca |
| Soluble | | ccgtgagcggagtgtccctcccagactacggagtgagctggattagacagcctcccggaaag |
| scFv-nt | | ggactggagtggatcggagtgatttggggtagcgaaaccacttactatcaatcttccctgaagtc |
| | | acgggtcaccatttcaaaggataactcaaagaatcaagtgagcctcaagctctcatcagtcac |
| | | cgccgctgacaccgccgtgtattactgtgccaagcattactactatggagggtcctacgccatgg |
| | | actactggggccagggaactctggtcactgtgtcatctggtggaggaggtagcggaggaggc |
| | | gggagcggtggaggtggctccgaaatcgtgatgacccagagccctgcaaccctgtccctttctc |
| | | ccggggaacgggctaccctttcttgtcgggcatcacaagatatctcaaaatacctcaattggtat |
| | | caacagaagccgggacaggcccctaggcttcttatctaccacacctctcgcctgcatagcggg |
| | | attcccgcacgctttagcgggtctggaagcgggaccgactacactctgaccatctcatctctcca |
| | | gcccgaggacttcgccgtctacttctgccagcagggtaacaccctgccgtacaccttcggccag |
| | | ggcaccaagcttgagatcaaacatcaccaccatcatcaccatcac |
| 103106 | 166 | MALPVTALLLPLALLLHAARPqvqlqesgpglvkpsetlsltctvsgvslpdygvs |
| CAR4- | | wirqppgkglewigviwgsettyyqsslksrvtiskdnsknqvslklssvtaadtavyycakhyy |
| Soluble | | yggsyamdywgqgtlvtvssggggsggggsggggseivmtqspatlslspgeratlscrasq |
| scFv-aa | | diskylnwyqqkpgqaprlliyhtsrlhsgiparfsgsgsgtdytltisslqpedfavyfcqqgntlp |
| | | ytfgqgtkleikhhhhhhhh |
| 104878 | 178 | atggctctgcccgtgaccgcactcctcctgccactggctctgctgcttcacgccgctcgcccaca |
| CAR 4- | | agtccagcttcaagaatcagggcctggtctggtgaagccatctgagactctgtccctcacttgca |
| Full-nt | | ccgtgagcggagtgtccccccagactacggagtgagctggattagacagcctcccggaaag |
| | | ggactggagtggatcggagtgatttggggtagcgaaaccacttactatcaatcttccctgaagtc |
| | | acgggtcaccatttcaaaggataactcaaagaatcaagtgagcctcaagctctcatcagtcac |
| | | cgccgctgacaccgccgtgtattactgtgccaagcattactactatggagggtcctacgccatgg |
| | | actactggggccagggaactctggtcactgtgtcatctggtggaggaggtagcggaggaggc |
| | | gggagcggtggaggtggctccgaaatcgtgatgacccagagccctgcaaccctgtccctttctc |
| | | ccggggaacgggctaccctttcttgtcgggcatcacaagatatctcaaaatacctcaattggtat |
| | | caacagaagccgggacaggcccctaggcttcttatctaccacacctctcgcctgcatagcggg |
| | | attcccgcacgctttagcgggtctggaagcgggaccgactacactctgaccatctcatctctcca |
| | | gcccgaggacttcgccgtctacttctgccagcagggtaacaccctgccgtacaccttcggccag |
| | | ggcaccaagcttgagatcaaaaccactactcccgctccaaggccacccacccctgccccgac |
| | | catcgcctctcagccgctttccctgcgtccggaggcatgtagacccgcagctggtggggccgtg |
| | | catacccggggtcttgacttcgcctgcgatatctacatttgggcccctctggctggtacttgcgggg |
| | | tcctgctgctttcactcgtgatcactctttactgtaagcgcggtcggaagaagctgctgtacatcttt |
| | | aagcaacccttcatgaggcctgtgcagactactcaagaggaggacggctgttcatgccggttc |
| | | ccagaggaggaggaaggcggctgcgaactgcgcgtgaaattcagccgcagcgcagatgct |
| | | ccagcctacaagcaggggcagaaccagctctacaacgaactcaatcttggtcggagagagg |
| | | agtacgacgtgctggacaagcggagaggacgggacccagaaatggggggaagccgcgc |
| | | agaaagaatccccaagagggcctgtacaacgagctccaaaaggataagatggcagaagcc |
| | | tatagcgagattggtatgaaaggggaacgcagaagaggcaaaggccacgacggactgtac |
| | | cagggactcagcaccgccaccaaggacacctatgacgctcttcacatgcaggccctgccgcc |
| | | tcgg |
| 104878 | 125 | MALPVTALLLPLALLLHAARPqvqlqesgpglvkpsetlsltctvsgvslpdygvswi |
| CAR 4- | | rqppgkglewigviwgsettyyqsslksrvtiskdnsknqvslklssvtaadtavyycakhyy |
| Full-aa | | yggsyamdywgqgtlvtvssggggsggggsggggseivmtqspatlslspgeratlscras |
| | | qdiskylnwyqqkpgqaprlliyhtsrlhsgiparfsgsgsgtdytltisslqpedfavyfcqqg |
| | | ntlpytfgqgtkleiktttpaprpptpaptiasqplsIrpeacrpaaggavhtrgldfacdiyiwapl |
| | | agtcgvlllslvitlyckrgrkkllyifkqpfmrpvqttqeedgcscrfpeeeeggcelrvkfsrsada |
| | | paykqgqnqlynelnlgrreeydvldkrrgrdpemggkprrknpqeglynelqkdkmaeays |
| | | eigmkgerrrgkghdglyqglstatkdtydalhmqalppr |

CAR 5

| | | |
|---|---|---|
| CAR5 | 100 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsggggsqvqlq |
| domain | | esgpglvkpsetlsltctvsgvslpdygvswirqppgkglewigviwgsettyyssslksrvtiskd |
| | | nsknqvslklssvtaadtavyycakhyyyggsyamdywgqgtlvtvss |
| 99789 | 155 | atggccctcccagtgaccgctctgctgctgcctctcgcacttcttctccatgccgctcggcctgag |
| CAR5- | | atcgtcatgacccaaagccccgctaccctgtccctgtcacccggcgagagggcaaccctttcat |
| Soluble | | gcagggccagccaggacatttctaagtacctcaactggtatcagcagaagccagggcaggct |
| scFv-nt | | cctcgcctgctgatctaccacaccagccgcctccacagcggtatccccgccagattttccggga |
| | | gcgggtctggaaccgactacaccctcaccatctcttctctgcagcccgaggatttcgccgtctattt |
| | | ctgccagcaggggaatactctgccgtacaccttcggtcaaggtaccaagctggaaatcaagg |
| | | gaggcggaggatcaggcggtggcggaagcggaggaggtggctccggaggaggaggttcc |
| | | caagtgcagcttcaagaatcaggacccggacttgtgaagccatcagaaaccctctccctgactt |
| | | gtaccgtgtccggtgtgagcctccccgactacggagtctcttggattcgccagcctccggggaa |
| | | gggtcttgaatggattggggtgatttggggatcagagactacttactactcttcatcacttaagtca |
| | | cgggtcaccatcagcaaagataatagcaagaaccaagtgtcacttaagctgtcatctgtgacc |
| | | gccgctgacaccgccgtgtactattgtgccaaacattactattacggagggtcttatgctatggac |
| | | tactggggacaggggaccctggtgactgtctctagccatcaccatcaccaccatcatcac |
| 99789 | 167 | MALPVTALLLPLALLLHAARPeivmtqspatlslspgeratlscrasqdiskylnwy |
| CAR5- | | qqkpgqaprlliyhtsrlhsgiparfsgsgsgtdytltisslqpedfavyfcqqgntlpytfgqgtklei |
| Soluble | | kggggsggggsggggsggggsqvqlqesgpglvkpsetlsltctvsgvslpdygvswirqpp |
| scFv-aa | | gkglewigviwgsettyyssslksrvtiskdnsknqvslklssvtaadtavyycakhyyyggsya |
| | | mdywgqgtlvtvsshhhhhhhh |
| 104879 | 179 | atggccctccctgtcaccgccctgctgcttccgctggctcttctgctccacgccgctcggcccgaa |
| CAR 5- | | attgtgatgacccagtcacccgccactcttagcctttcacccggtgagcgcgcaaccctgtcttgc |
| Full-nt | | agagcctcccaagacatctcaaaataccttaattggtatcaacagaagcccggacaggctcct |
| | | cgccttctgatctaccacaccagccggctccattctggaatccctgccaggttcagcggtagcgg |
| | | atctgggaccgactacaccctcactatcagctcactgcagccagaggacttcgctgtctatttctg |
| | | tcagcaagggaacaccctgccctacacctttggacagggcaccaagctcgagattaaaggtg |
| | | gaggtggcagcggaggaggtgggtccggcggtggaggaagcggcggaggcgggagcca |
| | | ggtccaactccaagaaagcggaccgggtcttgtgaagccatcagaaactctttcactgacttgt |
| | | actgtgagcggagtgtctctccccgattacggggtgtcttggatcagacagccaccggggaag |
| | | ggtctggaatggattggagtgatttggggctctgagactacttactactcttcatccctcaagtcac |
| | | gcgtcaccatctcaaaggacaactctaagaatcaggtgtcactgaaactgtcatctgtgaccgc |
| | | agccgacaccgccgtgtactattgcgctaagcattactattatggcgggagctacgcaatggatt |
| | | actggggacagggtactctggtcaccgtgtccagcaccactaccccagcaccgaggccaccc |
| | | accccggctcctaccatcgcctcccagcctctgtccctgcgtccggaggcatgtagacccgcag |
| | | ctggtggggccgtgcatacccggggtcttgacttcgcctgcgatatctacatttgggcccctctgg |
| | | ctggtacttgcggggtcctgctgctttcactcgtgatcactctttactgtaagcgcggtcggaagaa |
| | | gctgctgtacatctttaagcaacccttcatgaggcctgtgcagactactcaagaggaggacggc |
| | | tgttcatgccggttcccagaggaggaggaaggcggctgcgaactgcgcgtgaaattcagccg |
| | | cagcgcagatgctccagcctacaagcaggggcagaaccagctctacaacgaactcaatcttg |
| | | gtcggagagaggagtacgacgtgctggacaagcggagaggacgggacccagaaatgggc |
| | | gggaagccgcgcagaaagaatccccaagagggcctgtacaacgagctccaaaaggataa |
| | | gatggcagaagcctatagcgagattggtatgaaaggggaacgcagaagaggcaaaggcca |
| | | cgacggactgtaccagggactcagcaccgccaccaaggacacctatgacgctcttcacatgc |
| | | aggccctgccgcctcgg |
| 104879 | 126 | MALPVTALLLPLALLLHAARPeivmtqspatlslspgeratlscrasqdiskylnwy |
| CAR 5- | | qqkpgqaprlliyhtsrlhsgiparfsgsgsgtdytltisslqpedfavyfcqqgntlpytfgqgtkl |
| Full-aa | | eikggggsggggsggggggggsqvqlqesgpglvkpsetlsltctvsgvslpdygvswirq |
| | | ppgkglewigviwgsettyyssslksrvtiskdnsknqvslklssvtaadtavyycakhyyyg |
| | | gsyamdywgqgtlvtvsstttpaprpptpaptiasqplslrpeacrpaaggavhtrgldfacdiy |
| | | iwaplagtcgvlllslvitlyckrgrkkllyifkqpfmrpvqttqeedgcscrfpeeeeggcelrvkfsr |
| | | sadapaykqgqnqlynelnlgrreeydvldkrrgrdpemggkprrknpqeglynelqkdkma |
| | | eayseigmkgerrrgkghdglyqglstatkdtydalhmqalppr |

CAR 6

| | | |
|---|---|---|
| CAR6 | 101 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggggggsggggsggggsqvqlq |
| domain | | esgpglvkpsetlsltctvsgvslpdygvswirqppgkglewigviwgsettyyqsslksrvtiskd |
| | | nsknqvslklssvtaadtavyycakhyyyggsyamdywgqgtlvtvss |
| 99790 | 156 | atggccctcccagtgaccgctctgctgctgcctctcgcacttcttctccatgccgctcggcctgag |
| CAR6- | | atcgtcatgacccaaagccccgctaccctgtccctgtcacccggcgagagggcaaccctttcat |
| Soluble | | gcagggccagccaggacatttctaagtacctcaactggtatcagcagaagccagggcaggct |
| scFv-nt | | cctcgcctgctgatctaccacaccagccgcctccacagcggtatccccgccagattttccggga |
| | | gcgggtctggaaccgactacaccctcaccatctcttctctgcagcccgaggatttcgccgtctattt |
| | | ctgccagcaggggaatactctgccgtacaccttcggtcaaggtaccaagctggaaatcaagg |
| | | gaggcggaggatcaggcggtggcggaagcggaggaggtggctccggaggaggaggttcc |
| | | caagtgcagcttcaagaatcaggacccggacttgtgaagccatcagaaaccctctccctgactt |
| | | gtaccgtgtccggtgtgagcctccccgactacggagtctcttggattcgccagcctccggggaa |
| | | gggtcttgaatggattggggtgatttggggatcagagactacttactaccagtcatcacttaagtc |
| | | acgggtcaccatcagcaaagataatagcaagaaccaagtgtcacttaagctgtcatctgtgac |
| | | cgccgctgacaccgccgtgtactattgtgccaaacattactattacggagggtcttatgctatgga |
| | | ctactggggacaggggaccctggtgactgtctctagccatcaccatcaccaccatcatcac |
| 99790 | 168 | MALPVTALLLPLALLLHAARPeivmtqspatlslspgeratlscrasqdiskylnwy |
| CAR6- | | qqkpgqaprlliyhtsrlhsgiparfsgsgsgtdytltisslqpedfavyfcqqgntlpytfgqgtklei |
| Soluble | | kggggsggggggggsggggsqvqlqesgpglvkpsetlsltctvsgvslpdygvswirqpp |
| scFv-aa | | gkglewigviwgsettyyqsslksrvtiskdnsknqvslklssvtaadtavyycakhyyyggsya |
| | | mdywgqgtlvtvsshhhhhhhh |
| 104880 | 180 | atggccctccctgtcaccgccctgctgcttccgctggctcttctgctccacgccgctcggcccgaa |
| CAR6- | | attgtgatgacccagtcacccgccactcttagcctttcacccggtgagcgcgcaaccctgtcttgc |
| Full-nt | | agagcctcccaagacatctcaaaataccttaattggtatcaacagaagcccggacaggctcct |
| | | cgccttctgatctaccacaccagccggctccattctggaatccctgccaggttcagcggtagcgg |
| | | atctgggaccgactacaccctcactatcagctcactgcagccagaggacttcgctgtctatttctg |
| | | tcagcaagggaacaccctgccctacacctttggacagggcaccaagctcgagattaaaggtg |
| | | gaggtggcagcggaggaggtgggtccggcggtggaggaagcggaggcggagggagcca |
| | | ggtccaactccaagaaagcggaccgggtcttgtgaagccatcagaaactctttcactgacttgt |
| | | actgtgagcggagtgtctctccccgattacggggtgtcttggatcagacagccaccggggaag |
| | | ggtctggaatggattggagtgatttggggctctgagactacttactaccaatcatccctcaagtca |
| | | cgcgtcaccatctcaaaggacaactctaagaatcaggtgtcactgaaactgtcatctgtgaccg |
| | | cagccgacaccgccgtgtactattgcgctaagcattactattatggcgggagctacgcaatgga |
| | | ttactggggacagggtactctggtcaccgtgtccagcaccactaccccagcaccgaggccacc |
| | | caccccggctcctaccatcgcctcccagcctctgtccctgcgtccggaggcatgtagacccgca |
| | | gctggtggggccgtgcatacccggggtcttgacttcgcctgcgatatctacatttgggcccctctg |
| | | gctggtacttgcggggtcctgctgctttcactcgtgatcactctttactgtaagcgcggtcggaaga |
| | | agctgctgtacatctttaagcaacccttcatgaggcctgtgcagactactcaagaggaggacgg |
| | | ctgttcatgccggttcccagaggaggaggaaggcggctgcgaactgcgcgtgaaattcagcc |
| | | gcagcgcagatgctccagcctacaagcaggggcagaaccagctctacaacgaactcaatctt |
| | | ggtcggagagaggagtacgacgtgctggacaagcggagaggacgggacccagaaatggg |
| | | cgggaagccgcgcagaaagaatccccaagagggcctgtacaacgagctccaaaaggata |
| | | agatggcagaagcctatagcgagattggtatgaaaggggaacgcagaagaggcaaaggcc |
| | | acgacggactgtaccagggactcagcaccgccaccaaggacacctatgacgctcttcacatg |
| | | caggccctgccgcctcgg |
| 104880 | 127 | MALPVTALLLPLALLLHAARPeivmtqspatlslspgeratlscrasqdiskylnwy |
| CAR6- | | qqkpgqaprlliyhtsrlhsgiparfsgsgsgtdytltisslqpedfavyfcqqgntlpytfgqgtkl |
| Full-aa | | eikggggsggggsggggsggggsqvqlqesgpglvkpsetlsltctvsgvslpdygvswirq |
| | | ppgkglewigviwgsettyyqsslksrvtiskdnsknqvslklssvtaadtavyycakhyyy |
| | | ggsyamdywgqgtlvtvsstttpaprpptpaptiasqplslrpeacrpaaggavhtrgldfacdi |
| | | yiwaplagtcgvlllslvitlyckrgrkkllyifkqpfmrpvqttqeedgcscrfpeeeeggcelrvkfs |
| | | rsadapaykqgqnqlynelnlgrreeydvldkrrgrdpemggkprrknpqeglynelqkdkm |
| | | aeayseigmkgerrrgkghdglyqglstatkdtydalhmqalppr |

CAR 7

| | | |
|---|---|---|
| CAR7 | 102 | qvqlqesgpglvkpsetlsltctvsgvslpdygvswirqppgkglewigviwgsettyyssslksr |
| scFv | | vtiskdnsknqvslklssvtaadtavyycakhyyyggsyamdywgqgtlvtvssggggsggg |
| domain | | gsggggsggggseivmtqspatlslspgeratlscrasqdiskylnwyqqkpgqaprlliyhtsrl |
| | | hsgiparfsgsgsgtdytltisslqpedfavyfcqqgntlpytfgqgtkleik |
| 100796 | 157 | atggcactgcctgtcactgccctcctgctgcctctggccctccttctgcatgccgccaggccccaa |
| CAR7- | | gtccagctgcaagagtcaggacccggactggtgaagccgtctgagactctctcactgacttgta |
| Soluble | | ccgtcagcggcgtgtccctccccgactacggagtgtcatggatccgccaacctcccgggaaag |
| scFv-nt | | ggcttgaatggattggtgtcatctggggttctgaaaccacctactactcatcttccctgaagtccag |
| | | ggtgaccatcagcaaggataattccaagaaccaggtcagccttaagctgtcatctgtgaccgct |
| | | gctgacaccgccgtgtattactgcgccaagcactactattacggaggaagctacgctatggact |
| | | attggggacagggcactctcgtgactgtgagcagcggcggtggagggtctggaggtggagga |
| | | tccggtggtggtgggtcaggcggaggagggagcgagattgtgatgactcagtcaccagccac |
| | | cctttctctttcacccggcgagagagcaaccctgagctgtagagccagccaggacatttctaagt |
| | | acctcaactggtatcagcaaaaaccggggcaggcccctcgcctcctgatctaccatacctcac |
| | | gccttcactctggtatccccgctcggtttagcggatcaggatctggtaccgactacactctgacca |
| | | tttccagcctgcagccagaagatttcgcagtgtatttctgccagcagggcaatacccttccttaca |
| | | ccttcggtcagggaaccaagctcgaaatcaagcaccatcaccatcatcaccaccat |
| 100796 | 169 | MALPVTALLLPLALLLHAARPqvqlqesgpglvkpsetlsltctvsgvslpdygvs |
| CAR7- | | wirqppgkglewigviwgsettyyssslksrvtiskdnsknqvslklssvtaadtavyycakhyy |
| Soluble | | yggsyamdywgqgtlvtvssggggsggggsggggsggggseivmtqspatlslspgeratl |
| scFv-aa | | scrasqdiskylnwyqqkpgqaprlliyhtsrlhsgiparfsgsgsgtdytltisslqpedfavyfcq |
| | | qgntlpytfgqgtkleikhhhhhhhh |
| 104881 | 181 | atggctctgcccgtgaccgcactcctcctgccactggctctgctgcttcacgccgctcgcccaca |
| CAR 7 | | agtccagcttcaagaatcagggcctggtctggtgaagccatctgagactctgtccctcacttgca |
| Full-nt | | ccgtgagcggagtgtccctcccagactacggagtgagctggattagacagcctcccggaaag |
| | | ggactggagtggatcggagtgatttggggtagcgaaaccacttactattcatcttccctgaagtc |
| | | acgggtcaccatttcaaaggataactcaaagaatcaagtgagcctcaagctctcatcagtcac |
| | | cgccgctgacaccgccgtgtattactgtgccaagcattactactatggagggtcctacgccatgg |
| | | actactggggccagggaactctggtcactgtgtcatctggtggaggaggtagcggaggaggc |
| | | gggagcggtggaggtggctccggaggtggcggaagcgaaatcgtgatgacccagagccct |
| | | gcaaccctgtccctttctcccggggaacgggctaccctttcttgtcgggcatcacaagatatctca |
| | | aaatacctcaattggtatcaacagaagccgggacaggcccctaggcttcttatctaccacacct |
| | | ctcgcctgcatagcgggattcccgcacgctttagcgggtctggaagcgggaccgactacactct |
| | | gaccatctcatctctccagcccgaggacttcgccgtctacttctgccagcagggtaacaccctgc |
| | | cgtacaccttcggccagggcaccaagcttgagatcaaaaccactactcccgctccaaggcca |
| | | cccacccctgccccgaccatcgcctctcagccgctttccctgcgtccggaggcatgtagacccg |
| | | cagctggtggggccgtgcatacccggggtcttgacttcgcctgcgatatctacatttgggcccctc |
| | | tggctggtacttgcggggtcctgctgctttcactcgtgatcactctttactgtaagcgcggtcggaa |
| | | gaagctgctgtacatctttaagcaacccttcatgaggcctgtgcagactactcaagaggaggac |
| | | ggctgttcatgccggttcccagaggaggaggaaggcggctgcgaactgcgcgtgaaattcag |
| | | ccgcagcgcagatgctccagcctacaagcaggggcagaaccagctctacaacgaactcaat |
| | | cttggtcggagagaggagtacgacgtgctggacaagcggagaggacgggacccagaaatg |
| | | ggcgggaagccgcgcagaaagaatccccaagagggcctgtacaacgagctccaaaagga |
| | | taagatggcagaagcctatagcgagattggtatgaaaggggaacgcagaagaggcaaagg |
| | | ccacgacggactgtaccagggactcagcaccgccaccaaggacacctatgacgctcttcaca |
| | | tgcaggccctgccgcctcgg |
| 104881 | 128 | MALPVTALLLPLALLLHAARPqvqlqesgpglvkpsetlsltctvsgvslpdygvswi |
| CAR 7 | | rqppgkglewigviwgsettyyssslksrvtiskdnsknqvslklssvtaadtavyycakhyy |
| Full-aa | | yggsyamdywgqgtlvtvssggggsggggsggggsggggseivmtqspatlslspgeratl |
| | | scrasqdiskylnwyqqkpgqaprlliyhtsrlhsgiparfsgsgsgtdytltisslqpedfavyf |
| | | cqqgntlpytfgqgtkleiktttpaprpptpaptiasqplslrpeacrpaaggavhtrgldfacdiyi |
| | | waplagtcgvlllslvitlyckrgrkkllyifkqpfmrpvqttqeedgcscrfpeeeeggcelrvkfsr |
| | | sadapaykqgqnqlynelnlgrreeydvldkrrgrdpemggkprrknpqeglynelqkdkma |
| | | eayseigmkgerrrgkghdglyqglstatkdtydalhmqalppr |

CAR 8

| | | |
|---|---|---|
| CAR8 | 103 | qvqlqesgpglvkpsetlsltctvsgvslpdygvswirqppgkglewigviwgsettyyqsslksr |
| scFv | | vtiskdnsknqvslklssvtaadtavyycakhyyyggsyamdywgqgtlvtvssggggsggg |
| domain | | gsggggsggggseivmtqspatlslspgeratlscrasqdiskylnwyqqkpgqaprlliyhtsrl |
| | | hsgiparfsgsgsgtdytltisslqpedfavyfcqqgntlpytfgqgtkleik |
| 100798 | 158 | atggcactgcctgtcactgccctcctgctgcctctggccctccttctgcatgccgccaggccccaa |
| CAR8- | | gtccagctgcaagagtcaggacccggactggtgaagccgtctgagactctctcactgacttgta |
| Soluble | | ccgtcagcggcgtgtccctccccgactacggagtgtcatggatccgccaacctcccgggaaag |
| scFv-nt | | ggcttgaatggattggtgtcatctggggttctgaaaccacctactaccagtcttccctgaagtcca |
| | | gggtgaccatcagcaaggataattccaagaaccaggtcagccttaagctgtcatctgtgaccg |
| | | ctgctgacaccgccgtgtattactgcgccaagcactactattacggaggaagctacgctatgga |
| | | ctattggggacagggcactctcgtgactgtgagcagcggcggtggagggtctggaggtggag |
| | | gatccggtggtggtgggtcaggcggaggagggagcgagattgtgatgactcagtcaccagcc |
| | | accctttctctttcacccggcgagagagcaaccctgagctgtagagccagccaggacatttcta |
| | | agtacctcaactggtatcagcaaaaaccggggcaggcccctcgcctcctgatctaccatacctc |
| | | acgccttcactctggtatccccgctcggtttagcggatcaggatctggtaccgactacactctgac |
| | | catttccagcctgcagccagaagatttcgcagtgtatttctgccagcagggcaatacccttcctta |
| | | caccttcggtcagggaaccaagctcgaaatcaagcaccatcaccatcatcatcaccac |
| 100798 | 170 | MALPVTALLLPLALLLHAARPqvqlqesgpglvkpsetlsltctvsgvslpdygvs |
| CAR8- | | wirqppgkglewigviwgsettyyqsslksrvtiskdnsknqvslklssvtaadtavyycakhyy |
| Soluble | | yggsyamdywgqgtlvtvssggggsggggsggggsggggseivmtqspatlslspgeratl |
| scFv-aa | | scrasqdiskylnwyqqkpgqaprlliyhtsrlhsgiparfsgsgsgtdytltisslqpedfavyfcq |
| | | qgntlpytfgqgtkleikhhhhhhhh |
| 104882 | 182 | atggctctgcccgtgaccgcactcctcctgccactggctctgctgcttcacgccgctcgcccaca |
| CAR 8- | | agtccagcttcaagaatcagggcctggtctggtgaagccatctgagactctgtccctcacttgca |
| Full-nt | | ccgtgagcggagtgtccctcccagactacggagtgagctggattagacagcctcccggaaag |
| | | ggactggagtggatcggagtgatttggggtagcgaaaccacttactatcaatcttccctgaagtc |
| | | acgggtcaccatttcaaaggataactcaaagaatcaagtgagcctcaagctctcatcagtcac |
| | | cgccgctgacaccgccgtgtattactgtgccaagcattactactatggagggtcctacgccatgg |
| | | actactggggccagggaactctggtcactgtgtcatctggtggaggaggtagcggaggaggc |
| | | gggagcggtggaggtggctccggaggcggtgggtcagaaatcgtgatgacccagagccctg |
| | | caaccctgtccctttctcccggggaacgggctaccctttcttgtcgggcatcacaagatatctcaa |
| | | aatacctcaattggtatcaacagaagccgggacaggcccctaggcttcttatctaccacacctct |
| | | cgcctgcatagcgggattcccgcacgctttagcgggtctggaagcgggaccgactacactctg |
| | | accatctcatctctccagcccgaggacttcgccgtctacttctgccagcagggtaacaccctgcc |
| | | gtacaccttcggccagggcaccaagcttgagatcaaaaccactactcccgctccaaggccac |
| | | ccacccctgccccgaccatcgcctctcagccgctttccctgcgtccggaggcatgtagacccgc |
| | | agctggtggggccgtgcatacccggggtcttgacttcgcctgcgatatctacatttgggcccctct |
| | | ggctggtacttgcggggtcctgctgctttcactcgtgatcactctttactgtaagcgcggtcggaag |
| | | aagctgctgtacatctttaagcaacccttcatgaggcctgtgcagactactcaagaggaggacg |
| | | gctgttcatgccggttcccagaggaggaggaaggcggctgcgaactgcgcgtgaaattcagc |
| | | cgcagcgcagatgctccagcctacaagcaggggcagaaccagctctacaacgaactcaatc |
| | | ttggtcggagagaggagtacgacgtgctggacaagcggagaggacgggacccagaaatgg |
| | | gcgggaagccgcgcagaaagaatccccaagagggcctgtacaacgagctccaaaaggat |
| | | aagatggcagaagcctatagcgagattggtatgaaaggggaacgcagaagaggcaaagg |
| | | ccacgacggactgtaccagggactcagcaccgccaccaaggacacctatgacgctcttcaca |
| | | tgcaggccctgccgcctcgg |
| 104882 | 129 | MALPVTALLLPLALLLHAARPqvqlqesgpglvkpsetlsltctvsgvslpdygvswi |
| CAR 8- | | rqppgkglewigviwgsettyyqsslksrvtiskdnsknqvslklssvtaadtavyycakhyy |
| Full-aa | | yggsyamdywgqgtlvtvssggggsggggsggggsggggseivmtqspatlslspgeratl |
| | | scrasqdiskylnwyqqkpgqaprlliyhtsrlhsgiparfsgsgsgtdytltisslqpedfavyf |
| | | cqqgntlpytfgqgtkleiktttpaprpptpaptiasqplslrpeacrpaaggavhtrgldfacdiyi |
| | | waplagtcgvlllslvitlyckrgrkkllyifkqpfmrpvqttqeedgcscrfpeeeeggcelrvkfsr |
| | | sadapaykqgqnqlynelnlgrreeydvldkrrgrdpemggkprrknpqeglynelqkdkma |
| | | eayseigmkgerrrgkghdglyqglstatkdtydalhmqalppr |

CAR 9

| | | |
|---|---|---|
| CAR9 | 104 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggggggsggggsggggsqvqlq |
| domain | | esgpglvkpsetlsltctvsgvslpdygvswirqppgkglewigviwgsettyynsslksrvtiskd |
| | | nsknqvslklssvtaadtavyycakhyyyggsyamdywgqgtlvtvss |
| 99789 | 159 | atggccctcccagtgaccgctctgctgctgcctctcgcacttcttctccatgccgctcggcctgag |
| CAR9- | | atcgtcatgacccaaagccccgctaccctgtccctgtcacccggcgagagggcaaccctttcat |
| Soluble | | gcagggccagccaggacatttctaagtacctcaactggtatcagcagaagccagggcaggct |
| scFv-nt | | cctcgcctgctgatctaccacaccagccgcctccacagcggtatccccgccagattttccggga |
| | | gcgggtctggaaccgactacaccctcaccatctcttctctgcagcccgaggatttcgccgtctattt |
| | | ctgccagcaggggaatactctgccgtacaccttcggtcaaggtaccaagctggaaatcaagg |
| | | gaggcggaggatcaggcggtggcggaagcggaggaggtggctccggaggaggaggttcc |
| | | caagtgcagcttcaagaatcaggacccggacttgtgaagccatcagaaaccctctccctgactt |
| | | gtaccgtgtccggtgtgagcctccccgactacggagtctcttggattcgccagcctccggggaa |
| | | gggtcttgaatggattggggtgatttggggatcagagactacttactacaattcatcacttaagtca |
| | | cgggtcaccatcagcaaagataatagcaagaaccaagtgtcacttaagctgtcatctgtgacc |
| | | gccgctgacaccgccgtgtactattgtgccaaacattactattacggagggtcttatgctatggac |
| | | tactggggacaggggaccctggtgactgtctctagccatcaccatcaccaccatcatcac |
| 99789 | 171 | MALPVTALLLPLALLLHAARPeivmtqspatlslspgeratlscrasqdiskylnwy |
| CAR9- | | qqkpgqaprlliyhtsrlhsgiparfsgsgsgtdytltisslqpedfavyfcqqgntlpytfgqgtklei |
| Soluble | | kggggggggsggggsggggsqvqlqesgpglvkpsetlsltctvsgvslpdygvswirqpp |
| scFv-aa | | gkglewigviwgsettyynsslksrvtiskdnsknqvslklssvtaadtavyycakhyyyggsya |
| | | mdywgqgtlvtvsshhhhhhhh |
| 105974 | 183 | atggccctccctgtcaccgccctgctgcttccgctggctcttctgctccacgccgctcggcccgaa |
| CAR 9- | | attgtgatgacccagtcacccgccactcttagcctttcacccggtgagcgcgcaaccctgtcttgc |
| Full-nt | | agagcctcccaagacatctcaaaataccttaattggtatcaacagaagcccggacaggctcct |
| | | cgccttctgatctaccacaccagccggctccattctggaatccctgccaggttcagcggtagcgg |
| | | atctgggaccgactacaccctcactatcagctcactgcagccagaggacttcgctgtctatttctg |
| | | tcagcaagggaacaccctgccctacacctttggacagggcaccaagctcgagattaaaggtg |
| | | gaggtggcagcggaggaggtgggtccggcggtggaggaagcggaggcggtgggagccag |
| | | gtccaactccaagaaagcggaccgggtcttgtgaagccatcagaaactctttcactgacttgta |
| | | ctgtgagcggagtgtctctccccgattacggggtgtcttggatcagacagccaccggggaagg |
| | | gtctggaatggattggagtgatttggggctctgagactacttactacaactcatccctcaagtcac |
| | | gcgtcaccatctcaaaggacaactctaagaatcaggtgtcactgaaactgtcatctgtgaccgc |
| | | agccgacaccgccgtgtactattgcgctaagcattactattatggcgggagctacgcaatggatt |
| | | actggggacagggtactctggtcaccgtgtccagcaccactaccccagcaccgaggccaccc |
| | | accccggctcctaccatcgcctcccagcctctgtccctgcgtccggaggcatgtagacccgcag |
| | | ctggtggggccgtgcatacccggggtcttgacttcgcctgcgatatctacatttgggcccctctgg |
| | | ctggtacttgcggggtcctgctgctttcactcgtgatcactctttactgtaagcgcggtcggaagaa |
| | | gctgctgtacatctttaagcaacccttcatgaggcctgtgcagactactcaagaggaggacggc |
| | | tgttcatgccggttcccagaggaggaggaaggcggctgcgaactgcgcgtgaaattcagccg |
| | | cagcgcagatgctccagcctacaagcaggggcagaaccagctctacaacgaactcaatcttg |
| | | gtcggagagaggagtacgacgtgctggacaagcggagaggacgggacccagaaatgggc |
| | | gggaagccgcgcagaaagaatccccaagagggcctgtacaacgagctccaaaaggataa |
| | | gatggcagaagcctatagcgagattggtatgaaaggggaacgcagaagaggcaaaggcca |
| | | cgacggactgtaccagggactcagcaccgccaccaaggacacctatgacgctcttcacatgc |
| | | aggccctgccgcctcgg |
| 105974 | 130 | MALPVTALLLPLALLLHAARPeivmtqspatlslspgeratlscrasqdiskylnwy |
| CAR 9- | | qqkpgqaprlliyhtsrlhsgiparfsgsgsgtdytltisslqpedfavyfcqqgntlpytfgqgtkl |
| Full-aa | | eikggggggggsggggsggggsqvqlqesgpglvkpsetlsltctvsgvslpdygvswirq |
| | | ppgkglewigviwgsettyynsslksrvtiskdnsknqvslklssvtaadtavyycakhyyy |
| | | ggsyamdywgqgtlvtvsstttpaprpptpaptiasqplslrpeacrpaaggavhtrgldfacdi |
| | | yiwaplagtcgvlllslvitlyckrgrkkllyifkqpfmrpvqttqeedgcscrfpeeeeggcelrvkfs |
| | | rsadapaykqgqnqlynelnlgrreeydvldkrrgrdpemggkprrknpqeglynelqkdkm |
| | | aeayseigmkgerrrgkghdglyqglstatkdtydalhmqalppr |

CAR10

| | | |
|---|---|---|
| CAR10 | 105 | qvqlqesgpglvkpsetlsltctvsgvslpdygvswirqppgkglewigviwgsettyynsslksr |
| scFv | | vtiskdnsknqvslklssvtaadtavyycakhyyyggsyamdywgqgtlvtvssggggsggg |
| domain | | gsggggsggggseivmtqspatlslspgeratlscrasqdiskylnwyqqkpgqaprlliyhtsrl |
| | | hsgiparfsgsgsgtdytltisslqpedfavyfcqqgntlpytfgqgtkleik |
| 100796 | 160 | atggcactgcctgtcactgccctcctgctgcctctggccctccttctgcatgccgccaggccccaa |
| CAR10- | | gtccagctgcaagagtcaggacccggactggtgaagccgtctgagactctctcactgacttgta |
| Soluble | | ccgtcagcggcgtgtccctccccgactacggagtgtcatggatccgccaacctcccgggaaag |
| scFv-nt | | ggcttgaatggattggtgtcatctggggttctgaaaccacctactacaactcttccctgaagtcca |
| | | gggtgaccatcagcaaggataattccaagaaccaggtcagccttaagctgtcatctgtgaccg |
| | | ctgctgacaccgccgtgtattactgcgccaagcactactattacggaggaagctacgctatgga |
| | | ctattggggacagggcactctcgtgactgtgagcagcggcggtggagggtctggaggtggag |
| | | gatccggtggtggtgggtcaggcggaggagggagcgagattgtgatgactcagtcaccagcc |
| | | accctttctctttcacccggcgagagagcaaccctgagctgtagagccagccaggacatttcta |
| | | agtacctcaactggtatcagcaaaaaccggggcaggcccctcgcctcctgatctaccatacctc |
| | | acgccttcactctggtatccccgctcggtttagcggatcaggatctggtaccgactacactctgac |
| | | catttccagcctgcagccagaagatttcgcagtgtatttctgccagcagggcaatacccttcctta |
| | | caccttcggtcagggaaccaagctcgaaatcaagcaccatcaccatcatcaccaccat |
| 100796 | 172 | MALPVTALLLPLALLLHAARPqvqlqesgpglvkpsetlsltctvsgvslpdygvs |
| CAR10- | | wirqppgkglewigviwgsettyynsslksrvtiskdnsknqvslklssvtaadtavyycakhyy |
| Soluble | | yggsyamdywgqgtlvtvssggggsggggsggggsggggseivmtqspatlslspgeratl |
| scFv-aa | | scrasqdiskylnwyqqkpgqaprlliyhtsrlhsgiparfsgsgsgtdytltisslqpedfavyfcq |
| | | qgntlpytfgqgtkleikhhhhhhhh |
| 105975 | 184 | atggccctccctgtcaccgccctgctgcttccgctggctcttctgctccacgccgctcggcccgaa |
| CAR 10 | | attgtgatgacccagtcacccgccactcttagcctttcacccggtgagcgcgcaaccctgtcttgc |
| Full-nt | | agagcctcccaagacatctcaaaataccttaattggtatcaacagaagcccggacaggctcct |
| | | cgccttctgatctaccacaccagccggctccattctggaatccctgccaggttcagcggtagcgg |
| | | atctgggaccgactacaccctcactatcagctcactgcagccagaggacttcgctgtctatttctg |
| | | tcagcaagggaacaccctgccctacacctttggacagggcaccaagctcgagattaaaggtg |
| | | gaggtggcagcggaggaggtgggtccggcggtggaggaagcggaggcggtgggagccag |
| | | gtccaactccaagaaagcggaccgggtcttgtgaagccatcagaaactctttcactgacttgta |
| | | ctgtgagcggagtgtctctccccgattacggggtgtcttggatcagacagccaccggggaagg |
| | | gtctggaatggattggagtgatttggggctctgagactacttactacaactcatccctcaagtcac |
| | | gcgtcaccatctcaaaggacaactctaagaatcaggtgtcactgaaactgtcatctgtgaccgc |
| | | agccgacaccgccgtgtactattgcgctaagcattactattatggcgggagctacgcaatggatt |
| | | actggggacagggtactctggtcaccgtgtccagcaccactaccccagcaccgaggccaccc |
| | | accccggctcctaccatcgcctcccagcctctgtccctgcgtccggaggcatgtagacccgcag |
| | | ctggtggggccgtgcatacccggggtcttgacttcgcctgcgatatctacatttgggcccctctgg |
| | | ctggtacttgcggggtcctgctgctttcactcgtgatcactctttactgtaagcgcggtcggaagaa |
| | | gctgctgtacatctttaagcaacccttcatgaggcctgtgcagactactcaagaggaggacggc |
| | | tgttcatgccggttcccagaggaggaggaaggcggctgcgaactgcgcgtgaaattcagccg |
| | | cagcgcagatgctccagcctacaagcaggggcagaaccagctctacaacgaactcaatcttg |
| | | gtcggagagaggagtacgacgtgctggacaagcggagaggacgggacccagaaatgggc |
| | | gggaagccgcgcagaaagaatccccaagagggcctgtacaacgagctccaaaaggataa |
| | | gatggcagaagcctatagcgagattggtatgaaaggggaacgcagaagaggcaaaggcca |
| | | cgacggactgtaccagggactcagcaccgccaccaaggacacctatgacgctcttcacatgc |
| | | aggccctgccgcctcgg |
| 105975 | 131 | MALPVTALLLPLALLLHAARPEIVMTQSPATLSLSPGERATLSCRAS |
| CAR 10 | | QDISKYLNWYQQKPGQAPRLLIYHTSRLHSGIPARFSGSGSGTDYT |
| Full-aa | | LTISSLQPEDFAVYFCQQGNTLPYTFGQGTKLEIKGGGGSGGGGS |
| | | GGGGSGGGGSQVQLQESGPGLVKPSETLSLTCTVSGVSLPDYGV |
| | | SWIRQPPGKGLEWIGVIWGSETTYYNSSLKSRVTISKDNSKNQVSL |
| | | KLSSVTAADTAVYYCAKHYYYGGSYAMDYWGQGTLVTVSSTTTPA |
| | | PRPPTPAPTIASQPLSLRPEACRPAAGGAVHTRGLDFACDIYIWAPL |
| | | AGTCGVLLLSLVITLYCKRGRKKLLYIFKQPFMRPVQTTQEEDGCSC |
| | | RFPEEEEGGCELRVKFSRSADAPAYKQGQNQLYNELNLGRREEYD |
| | | VLDKRRGRDPEMGGKPRRKNPQEGLYNELQKDKMAEAYSEIGMK |
| | | GERRRGKGHDGLYQGLSTATKDTYDALHMQALPPR |

CAR11

| | | |
|---|---|---|
| CAR11 | 106 | eivmtqspatlslspgeratlscrasqdiskylnwyqqkpgqaprlliyhtsrlhsgiparfsgsgs |
| scFv | | gtdytltisslqpedfavyfcqqgntlpytfgqgtkleikggggsggggsggggsqvqlqesgpgl |
| domain | | vkpsetlsltctvsgvslpdygvswirqppgkglewigviwgsettyynsslksrvtiskdnsknq |
| | | vslklssvtaadtavyycakhyyyggsyamdywgqgtlvtvss |
| 103101 | 161 | Atggccctccctgtcaccgccctgctgcttccgctggctcttctgctccacgccgctcggcccgaa |
| CAR11- | | attgtgatgacccagtcacccgccactcttagcctttcacccggtgagcgcgcaaccctgtcttgc |
| Soluble | | agagcctcccaagacatctcaaaataccttaattggtatcaacagaagcccggacaggctcct |
| scFv-nt | | cgccttctgatctaccacaccagccggctccattctggaatccctgccaggttcagcggtagcgg |
| | | atctgggaccgactacaccctcactatcagctcactgcagccagaggacttcgctgtctatttctg |
| | | tcagcaagggaacaccctgccctacacctttggacagggcaccaagctcgagattaaaggtg |
| | | gaggtggcagcggaggaggtgggtccggcggtggaggaagccaggtccaactccaagaa |
| | | agcggaccgggtcttgtgaagccatcagaaactctttcactgacttgtactgtgagcggagtgtct |
| | | ctccccgattacggggtgtcttggatcagacagccaccggggaagggtctggaatggattgga |
| | | gtgatttggggctctgagactacttactacaattcatccctcaagtcacgcgtcaccatctcaaag |
| | | gacaactctaagaatcaggtgtcactgaaactgtcatctgtgaccgcagccgacaccgccgtgt |
| | | actattgcgctaagcattactattatgggggagctacgcaatggattactggggacagggtact |
| | | ctggtcaccgtgtccagccaccaccatcatcaccatcaccat |
| 103101 | 173 | MALPVTALLLPLALLLHAARPeivmtqspatlslspgeratlscrasqdiskylnwy |
| CAR11- | | qqkpgqaprlliyhtsrlhsgiparfsgsgsgtdytltisslqpedfavyfcqqgntlpytfgqgtklei |
| Soluble | | kggggsggggsggggsqvqlqesgpglvkpsetlsltctvsgvslpdygvswirqppgkglew |
| scFv-aa | | igviwgsettyynsslksrvtiskdnsknqvslklssvtaadtavyycakhyyyggsyamdywg |
| | | qgtlvtvsshhhhhhhh |
| 105976 | 185 | atggctctgcccgtgaccgcactcctcctgccactggctctgctgcttcacgccgctcgcccaca |
| CAR 11 | | agtccagcttcaagaatcagggcctggtctggtgaagccatctgagactctgtccctcacttgca |
| Full-nt | | ccgtgagcggagtgtccctcccagactacggagtgagctggattagacagcctcccggaaag |
| | | ggactggagtggatcggagtgatttggggtagcgaaaccacttactataactcttccctgaagtc |
| | | acgggtcaccatttcaaaggataactcaaagaatcaagtgagcctcaagctctcatcagtcac |
| | | cgccgctgacaccgccgtgtattactgtgccaagcattactactatggagggtcctacgccatgg |
| | | actactggggccagggaactctggtcactgtgtcatctggtggaggaggtagcggaggaggc |
| | | gggagcggtggaggtggctccggaggtggcggaagcgaaatcgtgatgacccagagccct |
| | | gcaaccctgtccctttctcccggggaacgggctaccctttcttgtcgggcatcacaagatatctca |
| | | aaatacctcaattggtatcaacagaagccgggacaggcccctaggcttcttatctaccacacct |
| | | ctcgcctgcatagcgggattcccgcacgctttagcgggtctggaagcgggaccgactacactct |
| | | gaccatctcatctctccagcccgaggacttcgccgtctacttctgccagcagggtaacaccctgc |
| | | cgtacaccttcggccagggcaccaagcttgagatcaaaaccactactcccgctccaaggcca |
| | | cccacccctgccccgaccatcgcctctcagccgctttccctgcgtccggaggcatgtagacccg |
| | | cagctggtggggccgtgcatacccggggtcttgacttcgcctgcgatatctacatttgggcccctc |
| | | tggctggtacttgcggggtcctgctgctttcactcgtgatcactctttactgtaagcgcggtcggaa |
| | | gaagctgctgtacatctttaagcaacccttcatgaggcctgtgcagactactcaagaggaggac |
| | | ggctgttcatgccggttcccagaggaggaggaaggcggctgcgaactgcgcgtgaaattcag |
| | | ccgcagcgcagatgctccagcctacaagcaggggcagaaccagctctacaacgaactcaat |
| | | cttggtcggagagaggagtacgacgtgctggacaagcggagaggacgggacccagaaatg |
| | | ggcgggaagccgcgcagaaagaatccccaagagggcctgtacaacgagctccaaaagga |
| | | taagatggcagaagcctatagcgagattggtatgaaaggggaacgcagaagaggcaaagg |
| | | ccacgacggactgtaccagggactcagcaccgccaccaaggacacctatgacgctcttcaca |
| | | tgcaggccctgccgcctcgg |
| 105976 | 132 | MALPVTALLLPLALLLHAARPQVQLQESGPGLVKPSETLSLTCTVSG |
| CAR 11 | | VSLPDYGVSWIRQPPGKGLEWIGVIWGSETTYYNSSLKSRVTISKD |
| Full-aa | | NSKNQVSLKLSSVTAADTAVYYCAKHYYYGGSYAMDYWGQGTLV |
| | | TVSSGGGGSGGGGSGGGGSGGGGSEIVMTQSPATLSLSPGERAT |
| | | LSCRASQDISKYLNWYQQKPGQAPRLLIYHTSRLHSGIPARFSGSG |
| | | SGTDYTLTISSLQPEDFAVYFCQQGNTLPYTFGQGTKLEIKTTTPAP |
| | | RPPTPAPTIASQPLSLRPEACRPAAGGAVHTRGLDFACDIYIWAPLA |
| | | GTCGVLLLSLVITLYCKRGRKKLLYIFKQPFMRPVQTTQEEDGCSCR |
| | | FPEEEEGGCELRVKFSRSADAPAYKQGQNQLYNELNLGRREEYDV |
| | | LDKRRGRDPEMGGKPRRKNPQEGLYNELQKDKMAEAYSEIGMKG |
| | | ERRRGKGHDGLYQGLSTATKDTYDALHMQALPPR |

CAR12

| | | |
|---|---|---|
| CAR12 | 107 | qvqlqesgpglvkpsetlsltctvsgvslpdygvswirqppgkglewigviwgsettyynsslksr |
| scFv | | vtiskdnsknqvslklssvtaadtavyycakhyyyggsyamdywgqgtlvtvssggggsggg |
| domain | | gsggggseivmtqspatlslspgeratlscrasqdiskylnwyqqkpgqaprlliyhtsrlhsgip |
| | | arfsgsgsgtdytltisslqpedfavyfcqqgntlpytfgqgtkleik |
| 103104 | 162 | atggctctgcccgtgaccgcactcctcctgccactggctctgctgcttcacgccgctcgcccaca |
| CAR12- | | agtccagcttcaagaatcagggcctggtctggtgaagccatctgagactctgtccctcacttgca |
| Soluble | | ccgtgagcggagtgtccctcccagactacggagtgagctggattagacagcctcccggaaag |
| scFv-nt | | ggactggagtggatcggagtgatttggggtagcgaaaccacttactataactcttccctgaagtc |
| | | acgggtcaccatttcaaaggataactcaaagaatcaagtgagcctcaagctctcatcagtcac |
| | | cgccgctgacaccgccgtgtattactgtgccaagcattactactatggagggtcctacgccatgg |
| | | actactggggccagggaactctggtcactgtgtcatctggtggaggaggtagcggaggaggc |
| | | gggagcggtggaggtggctccgaaatcgtgatgacccagagccctgcaaccctgtccctttctc |
| | | ccggggaacgggctaccctttcttgtcgggcatcacaagatatctcaaaatacctcaattggtat |
| | | caacagaagccgggacaggcccctaggcttcttatctaccacacctctcgcctgcatagcggg |
| | | attcccgcacgctttagcgggtctggaaggggaccgactacactctgaccatctcatctctcca |
| | | gcccgaggacttcgccgtctacttctgccagcagggtaacaccctgccgtacaccttcggccag |
| | | ggcaccaagcttgagatcaaacatcaccaccatcatcaccatcac |
| 103104 | 174 | MALPVTALLLPLALLLHAARPqvqlqesgpglvkpsetlsltctvsgvslpdygvs |
| CAR12- | | wirqppgkglewigviwgsettyynsslksrvtiskdnsknqvslklssvtaadtavyycakhyy |
| Soluble | | yggsyamdywgqgtlvtvssggggsggggsggggseivmtqspatlslspgeratlscrasq |
| scFv-aa | | diskylnwyqqkpgqaprlliyhtsrlhsgiparfsgsgsgtdytltisslqpedfavyfcqqgntlp |
| | | ytfgqgtkleikhhhhhhhh |
| 105977 | 186 | atggccctccctgtcaccgccctgctgcttccgctggctcttctgctccacgccgctcggcccgaa |
| CAR 12- | | attgtgatgacccagtcacccgccactcttagcctttcacccggtgagcgcgcaaccctgtcttgc |
| Full-nt | | agagcctcccaagacatctcaaaataccttaattggtatcaacagaagcccggacaggctcct |
| | | cgccttctgatctaccacaccagccggctccattctggaatccctgccaggttcagcggtagcgg |
| | | atctgggaccgactacaccctcactatcagctcactgcagccagaggacttcgctgtctatttctg |
| | | tcagcaagggaacaccctgccctacacctttggacagggcaccaagctcgagattaaaggtg |
| | | gaggtggcagcggaggaggtgggtccggcggtggaggaagccaggtccaactccaagaa |
| | | agcggaccgggtcttgtgaagccatcagaaactctttcactgacttgtactgtgagcggagtgtct |
| | | ctccccgattacggggtgtcttggatcagacagccaccggggaagggtctggaatggattgga |
| | | gtgatttggggctctgagactacttactacaactcatccctcaagtcacgcgtcaccatctcaaag |
| | | gacaactctaagaatcaggtgtcactgaaactgtcatctgtgaccgcagccgacaccgccgtgt |
| | | actattgcgctaagcattactattatggcgggagctacgcaatggattactggggacagggtact |
| | | ctggtcaccgtgtccagcaccactaccccagcaccgaggccacccaccccggctcctaccat |
| | | cgcctcccagcctctgtccctgcgtccggaggcatgtagacccgcagctggtggggccgtgcat |
| | | acccggggtcttgacttcgcctgcgatatctacatttgggcccctctggctggtacttgcggggtcc |
| | | tgctgctttcactcgtgatcactctttactgtaagcgcggtcggaagaagctgctgtacatctttaag |
| | | caacccttcatgaggcctgtgcagactactcaagaggaggacggctgttcatgccggttcccag |
| | | aggaggaggaaggcggctgcgaactgcgcgtgaaattcagccgcagcgcagatgctccag |
| | | cctacaagcaggggcagaaccagctctacaacgaactcaatcttggtcggagagaggagta |
| | | cgacgtgctggacaagcggagaggacgggacccagaaatgggcgggaagccgcgcaga |
| | | aagaatccccaagagggcctgtacaacgagctccaaaaggataagatggcagaagcctata |
| | | gcgagattggtatgaaaggggaacgcagaagaggcaaaggccacgacggactgtaccag |
| | | ggactcagcaccgccaccaaggacacctatgacgctcttcacatgcaggccctgccgcctcg |
| | | g |
| 105977 | 133 | MALPVTALLLPLALLLHAARPEIVMTQSPATLSLSPGERATLSCRAS |
| CAR 12- | | QDISKYLNWYQQKPGQAPRLLIYHTSRLHSGIPARFSGSGSGTDYT |
| Full-aa | | LTISSLQPEDFAVYFCQQGNTLPYTFGQGTKLEIKGGGGSGGGGS |
| | | GGGGSQVQLQESGPGLVKPSETLSLTCTVSGVSLPDYGVSWIRQP |
| | | PGKGLEWIGVIWGSETTYYNSSLKSRVTISKDNSKNQVSLKLSSVT |
| | | AADTAVYYCAKHYYYGGSYAMDYWGQGTLVTVSSTTTPAPRPPTP |
| | | APTIASQPLSLRPEACRPAAGGAVHTRGLDFACDIYIWAPLAGTCG |
| | | VLLLSLVITLYCKRGRKKLLYIFKQPFMRPVQTTQEEDGCSCRFPEE |
| | | EEGGCELRVKFSRSADAPAYKQGQNQLYNELNLGRREEYDVLDKR |
| | | RGRDPEMGGKPRRKNPQEGLYNELQKDKMAEAYSEIGMKGERRR |
| | | GKGHDGLYQGLSTATKDTYDALHMQALPPR |

Exemplary ful CAR constructs having scFv domains SEQ ID NOs: 96-107 are shown in SEQ ID NOs: 122-133.

The sequences of the murine scFv fragments (SEQ ID NOS: 188, 194, 196 and 199) are provided below in Table 17.

TABLE 17
Murine CD19 CAR Constructs

| Name | | |
|---|---|---|
| CTL019 | SEQ ID | Sequence |
| CTL019- | 187 | Atggccctgcccgtcaccgctctgctgctgccccttgctctgcttcttcatgcagcaaggccggac |
| Soluble | | atccagatgacccaaaccacctcatccctctctgcctctcttggagacagggtgaccatttcttgtc |
| scFv- | | gcgccagccaggacatcagcaagtatctgaactggtatcagcagaagccggacggaaccgt |
| Histag-nt | | gaagctcctgatctaccatacctctcgcctgcatagcggcgtgccctcacgcttctctggaagcg |
| | | gatcaggaaccgattattctctcactatttcaaatcttgagcaggaagatattgccacctatttctgc |
| | | cagcagggtaataccctgccctacaccttcggaggagggaccaagctcgaaatcaccggtgg |
| | | aggaggcagcggcggtggagggtctggtggaggtggttctgaggtgaagctgcaagaatca |
| | | ggccctggacttgtggccccttcacagtccctgagcgtgacttgcaccgtgtccggagtctccctg |
| | | cccgactacggagtgtcatggatcagacaacctccacggaaaggactggaatggctcggtgt |
| | | catctggggtagcgaaactacttactacaattcagccctcaaaagcaggctgactattatcaag |
| | | gacaacagcaagtcccaagtctttcttaagatgaactcactccagactgacgacaccgcaatct |
| | | actattgtgctaagcactactactacggaggatcctacgctatggattactggggacaaggtactt |
| | | ccgtcactgtctcttcacaccatcatcaccatcaccatcac |
| CTL019- | 188 | MALPVTALLLPLALLLHAARPdiqmtqttsslsaslgdrvtiscrasqdiskylnwyq |
| Soluble | | qkpdgtvklliyhtsrlhsgvpsrfsgsgsgtdysltisnleqediatyfcqqgntlpytfgggtkleit |
| scFv- | | ggggsggggsggggsevklqesgpglvapsqslsvtctvsgvslpdygvswirqpprkglewl |
| Histag-aa | | gviwgsettyynsalksrltiikdnsksqvflkmnslqtddtaiyycakhyyyggsyamdywgq |
| | | gtsvtvsshhhhhhhh |
| CTL019 | 189 | atggccttaccagtgaccgccttgctcctgccgctggccttgctgctccacgccgccaggccgg |
| Full-nt | | acatccagatgacacagactacatcctccctgtctgcctctctgggagacagagtcaccatcag |
| | | ttgcagggcaagtcaggacattagtaaatatttaaattggtatcagcagaaaccagatggaact |
| | | gttaaactcctgatctaccatacatcaagattacactcaggagtcccatcaaggttcagtggcag |
| | | tgggtctggaacagattattctctcaccattagcaacctggagcaagaagatattgccacttacttt |
| | | tgccaacagggtaatacgcttccgtacacgttcggaggggggaccaagctggagatcacagg |
| | | tggcggtggctcgggcggtggtgggtcgggtggcggcggatctgaggtgaaactgcaggagt |
| | | caggacctggcctggtggcgccctcacagagcctgtccgtcacatgcactgtctcaggggtctc |
| | | attacccgactatggtgtaagctggattcgccagcctccacgaaagggtctggagtggctggga |
| | | gtaatatggggtagtgaaaccacatactataattcagctctcaaatccagactgaccatcatcaa |
| | | ggacaactccaagagccaagttttcttaaaaatgaacagtctgcaaactgatgacacagccatt |
| | | tactactgtgccaaacattattactacggtggtagctatgctatggactactggggccaaggaac |
| | | ctcagtcaccgtctcctcaaccacgacgccagcgccgcgaccaccaacaccggcgcccacc |
| | | atcgcgtcgcagcccctgtccctgcgcccagaggcgtgccggccagcggcggggggcgcag |
| | | tgcacacgagggggctggacttcgcctgtgatatctacatctgggcgcccttggccgggacttgt |
| | | ggggtccttctcctgtcactggttatcaccctttactgcaaacggggcagaaagaaactcctgtat |
| | | atattcaaacaaccatttatgagaccagtacaaactactcaagaggaagatggctgtagctgcc |
| | | gatttccagaagaagaagaaggaggatgtgaactgagagtgaagttcagcaggagcgcag |
| | | acgcccccgcgtacaagcagggccagaaccagctctataacgagctcaatctaggacgaag |
| | | agaggagtacgatgttttggacaagagacgtggccgggaccctgagatggggggaaagccg |
| | | agaaggaagaaccctcaggaaggcctgtacaatgaactgcagaaagataagatggcggag |
| | | gcctacagtgagattgggatgaaaggcgagcgccggaggggcaaggggcacgatggccttt |
| | | accagggtctcagtacagccaccaaggacacctacgacgcccttcacatgcaggccctgccc |
| | | cctcgc |
| CTL019 | 148 | MALPVTALLLPLALLLHAARPdiqmtqttsslsaslgdrvtiscrasqdiskylnwyqq |
| Full-aa | | kpdgtvklliyhtsrlhsgvpsrfsgsgsgtdysltisnleqediatyfcqqgntlpytfgggtkleitg |
| | | gggsggggsggggsevklqesgpglvapsqslsvtctvsgvslpdygvswirqpprkglewlg |
| | | viwgsettyynsalksrltiikdnsksqvflkmnslqtddtaiyycakhyyyggsyamdywgqgt |
| | | svtvsstttpaprpptpaptiasqplslrpeacrpaaggavhtrgldfacdiyiwaplagtcgvlllsl |
| | | vitlyckrgrkkllyifkqpfmrpvqttqeedgcscrfpeeeeggcelrvkfsrsadapaykqgqn |
| | | qlynelnlgrreeydvldkrrgrdpemggkprrknpqeglynelqkdkmaeayseigmkgerr |
| | | rgkghdglyqglstatkdtydalhmqalppr |
| CTL019 | 149 | Diqmtqttsslsaslgdrvtiscrasqdiskylnwyqqkpdgtvklliyhtsrlhsgvpsrfsgsgs |
| scFv | | gtdysltisnleqediatyfcqqgntlpytfgggtkleitggggsggggsggggsevklqesgpgl |
| domain | | vapsqslsvtctvsgvslpdygvswirqpprkglewlgviwgsettyynsalksrltiikdnsksqv |
| | | flkmnslqtddtaiyycakhyyyggsyamdywgqgtsvtvss |
| mCAR1 | 194 | QVQLLESGAELVRPGSSVKISCKASGYAFSSYWMNWVKQRPGQG |
| scFv | | LEWIGQIYPGDGDTNYNGKFKGQATLTADKSSSTAYMQLSGLTSED |
| | | SAVYSCARKTISSVVDFYFDYWGQGTTVTGGGSGGGSGGGSGGG |
| | | SELVLTQSPKFMSTSVGDRVSVTCKASQNVGTNVAWYQQKPGQS |
| | | PKPLIYSATYRNSGVPDRFTGSGSGTDFTLTITNVQSKDLADYFCQY |
| | | NRYPYTSFFFTKLEIKRRS |
| mCAR1 | 195 | QVQLLESGAELVRPGSSVKISCKASGYAFSSYWMNWVKQRPGQG |
| Full-aa | | LEWIGQIYPGDGDTNYNGKFKGQATLTADKSSSTAYMQLSGLTSED |
| | | SAVYSCARKTISSVVDFYFDYWGQGTTVTGGGSGGGSGGGSGGG |
| | | SELVLTQSPKFMSTSVGDRVSVTCKASQNVGTNVAWYQQKPGQS |
| | | PKPLIYSATYRNSGVPDRFTGSGSGTDFTLTITNVQSKDLADYFCQY |
| | | NRYPYTSFFFTKLEIKRRSKIEVMYPPPYLDNEKSNGTIIHVKGKHLC |
| | | PSPLFPGPSKPFWVLVVVGGVLACYSLLVTVAFIIFWVRSKRSRLLH |
| | | SDYMNMTPRRPGPTRKHYQPYAPPRDFAAYRSRVKFSRSADAPA |
| | | YQQGQNQLYNELNLGRREEYDVLDKRRGRDPEMGGKPRRKNPQ |
| | | EGLYNELQKDKMAEAYSEIGMKGERRRGKGHDGLYQGLSTATKDT |
| | | YDALHMQALPPR |
| mCAR2 | 196 | DIQMTQTTSSLSASLGDRVTISCRASQDISKYLNWYQQKPDGTVKL |
| scFv | | LIYHTSRLHSGVPSRFSGSGSGTDYSLTISNLEQEDIATYFCQQGNT |
| | | LPYTFGGGTKLEITGSTSGSGKPGSGEGSTKGEVKLQESGPGLVA |
| | | PSQSLSVTCTVSGVSLPDYGVSWIRQPPRKGLEWLGVIWGSETTY |
| | | YNSALKSRLTIIKDNSKSQVFLKMNSLQTDDTAIYYCAKHYYYGGSY |
| | | AMDYWGQGTSVTVSSE |
| mCAR2 | 197 | DIQMTQTTSSLSASLGDRVTISCRASQDISKYLNWYQQKPDGTVKL |
| CAR-aa | | LIYHTSRLHSGVPSRFSGSGSGTDYSLTISNLEQEDIATYFCQQGNT |
| | | LPYTFGGGTKLEITGSTSGSGKPGSGEGSTKGEVKLQESGPGLVA |
| | | PSQSLSVTCTVSGVSLPDYGVSWIRQPPRKGLEWLGVIWGSETTY |
| | | YNSALKSRLTIIKDNSKSQVFLKMNSLQTDDTAIY |
| | | YCAKHYYYGGSYAMDYWGQGTSVTVSSESKYGPPCPPCPMFWVL |
| | | VVVGGVLACYSLLVTVAFIIFWVKRGRKKLLYIFKQPFMRPVQTTQE |
| | | EDGCSCRFEEEEGGCELRVKFSRSADAPAYQQGQNQLYNELNLG |
| | | RREEYDVLDKRRGRDPEMGGKPRRKNPQEGLYNELQKDKMAEAY |
| | | SEIGMKGERRRGKGHDGLYQGLSTATKDTYDALHMQALPPRL |
| mCAR2 | 198 | DIQMTQTTSSLSASLGDRVTISCRASQDISKYLNWYQQKPDGTVKL |
| Full-aa | | LIYHTSRLHSGVPSRFSGSGSGTDYSLTISNLEQEDIATYFCQQGNT |
| | | LPYTFGGGTKLEITGSTSGSGKPGSGEGSTKGEVKLQESGPGLVA |
| | | PSQSLSVTCTVSGVSLPDYGVSWIRQPPRKGLEWLGVIWGSETTY |
| | | YNSALKSRLTIIKDNSKSQVFLKMNSLQTDDTAIYYCAKHYYYGGSY |
| | | AMDYWGQGTSVTVSSESKYGPPCPPCPMFWVLVVVGGVLACYSL |
| | | LVTVAFIIFWVKRGRKKLLYIFKQPFMRPVQTTQEEDGCSCRFEEEE |
| | | GGCELRVKFSRSADAPAYQQGQNQLYNELNLGRREEYDVLDKRR |
| | | GRDPEMGGKPRRKNPQEGLYNELQKDKMAEAYSEIGMKGERRRG |
| | | KGHDGLYQGLSTATKDTYDALHMQALPPRLEGGGEGRGSLLTCGD |
| | | VEENPGPRMLLLVTSLLLCELPHPAFLLIPRKVCNGIGIGEFKDSLSI |
| | | NATNIKHFKNCTSISGDLHILPVAFRGDSFTHTPPLDPQELDILKTVK |
| | | EITGFLLIQAWPENRTDLHAFENLEIIRGRTKQHGQFSLAVVSLNITS |
| | | LGLRSLKEISDGDVIISGNKNLCYANTINWKKLFGTSGQKTKIISNRG |
| | | ENSCKATGQVCHALCSPEGCWGPEPRDCVSCRNVSRGRECVDK |
| | | CNLLEGEPREFVENSECIQCHPECLPQAMNITCTGRGPDNCIQCAH |
| | | YIDGPHCVKTCPAGVMGENNTLVWKYADAGHVCHLCHPNCTYGC |
| | | TGPGLEGCPTNGPKIPSIATGMVGALLLLLVVALGIGLFM |
| mCAR3 | 199 | DIQMTQTTSSLSASLGDRVTISCRASQDISKYLNWYQQKPDGTVKL |
| scFv | | LIYHTSRLHSGVPSRFSGSGSGTDYSLTISNLEQEDIATYFCQQGNT |
| | | LPYTFGGGTKLEITGSTSGSGKPGSGEGSTKGEVKLQESGPGLVA |
| | | PSQSLSVTCTVSGVSLPDYGVSWIRQPPRKGLEWLGVIWGSETTY |
| | | YNSALKSRLTIIKDNSKSQVFLKMNSLQTDDTAIYYCAKHYYYGGSY |
| | | AMDYWGQGTSVTVSS |
| mCAR3 | 200 | DIQMTQTTSSLSASLGDRVTISCRASQDISKYLNWYQQKPDGTVKL |
| Full-aa | | LIYHTSRLHSGVPSRFSGSGSGTDYSLTISNLEQEDIATYFCQQGNT |
| | | LPYTFGGGTKLEITGSTSGSGKPGSGEGSTKGEVKLQESGPGLVA |
| | | PSQSLSVTCTVSGVSLPDYGVSWIRQPPRKGLEWLGVIWGSETTY |
| | | YNSALKSRLTIIKDNSKSQVFLKMNSLQTDDTAIYYCAKHYYYGGSY |
| | | AMDYWGQGTSVTVSSAAAIEVMYPPPYLDNEKSNGTIIHVKGKHLC |
| | | PSPLFPGPSKPFWVLVVVGGVLACYSLLVTVAFIIFWVRSKRSRLLH |
| | | SDYMNMTPRRPGPTRKHYQPYAPPRDFAAYRSRVKFSRSADAPA |
| | | YQQGQNQLYNELNLGRREEYDVLDKRRGRDPEMGGKPRRKNPQ |
| | | EGLYNELQKDKMAEAYSEIGMKGERRRGKGHDGLYQGLSTATKDT |
| | | YDALHMQALPPR |
| SSJ25-C1 | 216 | QVQLLESGAELVRPGSSVKISCKASGYAFSSYWMNWVKQRPGQG |
| VH | | LEWIGQIYPGDGDTNYNGKFKGQATLTADKSSSTAYMQLSGLTSED |
| sequence | | SAVYSCARKTISSVVDFYFDYWGQGTTVT |
| SSJ25-C1 | 217 | ELVLTQSPKFMSTSVGDRVSVTCKASQNVGTNVAWYQQKPGQSP |
| VL | | KPLIYSATYRNSGVPDRFTGSGSGTDFTLTITNVQSKDLADYFYFCQ |
| sequence | | YNRYPYTSGGGTKLEIKRRS |

Full CAR constructs using SEQ ID NOs: 188, 194, 196 and 209 are shown in SEQ ID NOs: 148, 195, 197, 198 and 200.

The present disclosure encompasses the use of CAR molecules of any one of SEQ ID NOs:122-133, 148, 195, 197, 198 and 200 in the methods and combinations of the disclosure. In specific aspects, a CAR construct of the disclosure comprises a scFv domain selected from the group consisting of SEQ ID NOS:96-107 or an scFV domain of SEQ ID NO:149, wherein the scFv may be preceded by an optional leader sequence, and followed by an optional hinge sequence, a transmembrane region and a CD3-zeta sequence, wherein the domains are contiguous with and in the same reading frame to form a single fusion protein.

A CAR molecule construct of the disclosure can be encoded by a nucleic acid construct comprising the nucleotide sequence of any one of SEQ ID NOS:175-189. In one aspect the nucleic acid sequence of a CAR construct comprises SEQ ID NO:175. In one aspect the nucleic acid sequence of a CAR construct comprises SEQ ID NO:176. In one aspect the nucleic acid sequence of a CAR construct comprises SEQ ID NO:177. In one aspect the nucleic acid sequence of a CAR construct comprises SEQ ID NO:178. In one aspect the nucleic acid sequence of a CAR construct comprises SEQ ID NO:179. In one aspect the nucleic acid sequence of a CAR construct comprises SEQ ID NO:180. In one aspect the nucleic acid sequence of a CAR construct comprises SEQ ID NO:181. In one aspect the nucleic acid sequence of a CAR construct comprises SEQ ID NO:182. In one aspect the nucleic acid sequence of a CAR construct comprises SEQ ID NO:183. In one aspect the nucleic acid sequence of a CAR construct comprises SEQ ID NO:184. In one aspect the nucleic acid sequence of a CAR construct comprises SEQ ID NO:185. In one aspect the nucleic acid sequence of a CAR construct comprises SEQ ID NO:186. In one aspect the nucleic acid sequence of a CAR construct is SEQ ID NO:187. In one aspect the nucleic acid sequence of a CAR construct comprises SEQ ID NO:188. In one aspect the nucleic acid sequence of a CAR construct comprises SEQ ID NO:189.

Full-length CAR sequences are also provided herein as SEQ ID NOS: 122-133 and 148, as shown in Table 16 (e.g., CTL119) and Table 17 (e.g., CTL019).

Exemplary sequences of various scFv fragments and other CAR components are provided herein. It is noted that these CAR components without a leader sequence, are also provided herein.

In one aspect, a CAR molecule is encoded by a nucleic acid molecule comprising the nucleic acid sequence encoding an anti-CD19 binding domain, e.g., described herein, that is contiguous with and in the same reading frame as a nucleic acid sequence encoding an intracellular signaling domain. In one aspect, the anti-CD19 binding domain is selected from one or more of SEQ ID NOS:96-107 and 148. In one aspect, the anti-CD19 binding domain is encoded by a nucleotide residues 64 to 813 of the sequence provided in one or more of SEQ ID NOS:151-166 and 187. In one aspect, the anti-CD19 binding domain is encoded by a nucleotide residues 64 to 813 of SEQ ID NO:156. In one aspect, the anti-CD19 binding domain is encoded by a nucleotide residues 64 to 813 of SEQ ID NO:152. In one aspect, the anti-CD19 binding domain is encoded by a nucleotide residues 64 to 813 of SEQ ID NO:153. In one aspect, the anti-CD19 binding domain is encoded by a nucleotide residues 64 to 813 of SEQ ID NO:154. In one aspect, the anti-CD19 binding domain is encoded by a nucleotide residues 64 to 813 of SEQ ID NO:155. In one aspect, the anti-CD19 binding domain is encoded by a nucleotide residues 64 to 813 of SEQ ID NO:156. In one aspect, the anti-CD19 binding domain is encoded by a nucleotide residues 64 to 813 of SEQ ID NO:157. In one aspect, the anti-CD19 binding domain is encoded by a nucleotide residues 64 to 813 of SEQ ID NO:158. In one aspect, the anti-CD19 binding domain is encoded by a nucleotide residues 64 to 813 of SEQ ID NO:159. In one aspect, the anti-CD19 binding domain is encoded by a nucleotide residues 64 to 813 of SEQ ID NO:160. In one aspect, the anti-CD19 binding domain is encoded by a nucleotide residues 64 to 813 of SEQ ID NO:161. In one aspect, the anti-CD19 binding domain is encoded by a nucleotide residues 64 to 813 of SEQ ID NO:162.

In further aspects a CAR molecule comprises an amino acid sequence having at least 85, 90, 95, 96, 97, 98, 99 or 100% identity to the scFv portion of 1928z (see, e.g., U.S. Pat. No. 10,124,023, which is incorporated by reference herein) and/or has the amino acid sequences of the heavy and light chain CDRs of 1928z. In some embodiments, the CD19 CAR molecule comprises the entire amino acid sequence of the 1928z (with or without its leader sequence) or an amino acid sequence having at least 85, 90, 95, 96, 97, 98, 99 or 100% identity to the sequence of 1928z (with or without its leader sequence), reproduced below:

(SEQ ID NO: 201)
MALPVTALLLPLALLLHAEVKLQQSGAELVRPGSSVKISCKASGYAFSS
YWMNWVKQRPGQGLEWIGQIYPGDGDTNYNGKFKGQATLTADKSSSTAY
MQLSGLTSEDSAVYFCARKTISSVVDFYFDYWGQGTTVTVSSGGGGSGG
GGSGGGGSDIELTQSPKFMSTSVGDRVSVTCKASQNVGTNVAWYQQKPG
QSPKPLIYSATYRNSGVPDRFTGSGSGTDFTLTITNVQSKDLADYFCQQ
YNRYPYTSGGGTKLEIKRAAAIEVMYPPPYLDNEKSNGTIIHVKGKHLC
PSPLFPGPSKPFWVLVVVGGVLACYSLLVTVAFIIFWVRSKRSRLLHSD
YMNMTPRRPGPTRKHYQPYAPPRDFAAYRSRVKFSRSAEPPAYQQGQNQ
LYNELNLGRREEYDVLDKRRGRDPEMGGKPRRKNPQEGLYNELQKDKMA
EAYSEIGMKGERRRGKGHDGLYQGLSTATKDTYDALHMQALPPRX

An exemplary nucleic acid sequence encoding a 1928z polypeptide of SEQ ID NO:201 is reproduced below:

(SEQ ID NO: 202)
ccatggctctcccagtgactgccctactgcttcccctagcgcttctcct
gcatgcagaggtgaagctgcagcagtctggggctgagctggtgaggcct
gggtcctcagtgaagatttcctgcaaggcttctggctatgcattcagta
gctactggatgaactgggtgaagcagaggcctggacagggtcttgagtg
gattggacagatttatcctggagatggtgatactaactacaatggaaag
ttcaagggtcaagccacactgactgcagacaaatcctccagcacagcct
acatgcagctcagcggcctaacatctgaggactctgcggtctatttctg
tgcaagaaagaccattagttcggtagtagatttctactttgactactgg
ggccaagggaccacggtcaccgtctcctcaggtggaggtggatcaggtg
gaggtggatctggtggaggtggatctgacattgagctcacccagtctcc
aaaattcatgtccacatcagtaggagacagggtcagcgtcacctgcaag
gccagtcagaatgtgggtactaatgtagcctggtatcaacagaaaccag
gacaatctcctaaaccactgatttactcggcaacctaccggaacagtgg
agtccctgatcgcttcacaggcagtggatctgggacagatttcactctc
accatcactaacgtgcagtctaaagacttggcagactatttctgtcaac
aatataacaggtatccgtacacgtccggaggggggaccaagctggagat
caaacgggcggccgcaattgaagttatgtatcctcctccttacctagac
aatgagaagagcaatggaaccattatccatgtgaaagggaaacaccttt
gtccaagtcccctatttcccggaccttctaagcccttttgggtgctggt
ggtggttggtggagtcctggcttgctatagcttgctagtaacagtggcc
tttattattttctgggtgaggagtaagaggagcaggctcctgcacagtg
actacatgaacatgactccccgccgccccgggcccacccgcaagcatta
ccagccctatgccccaccacgcgacttcgcagcctatcgctccagagtg
aagttcagcaggagcgcagagccccccgcgtaccagcagggccagaacc
agctctataacgagctcaatctaggacgaagagaggagtacgatgtttt
ggacaagagacgtggccgggaccctgagatggggggaaagccgagaagg
aagaaccctcaggaaggcctgtacaatgaactgcagaaagataagatgg
cggaggcctacagtgagattgggatgaaaggcgagcgccggaggggcaa
ggggcacgatggcctttaccagggtctcagtacagccaccaaggacacc
tacgacgcccttcacatgcaggccctgccccctcgcg

### 7.3.5. CAR Encoding Nucleic Acids

Nucleic acid molecules encoding the CAR molecules useful for the methods disclosed herein, for example the CAR molecules described in Section 7.3.4 can be obtained using recombinant methods known in the art, such as, for example by screening libraries from cells expressing the nucleic acid molecule, by deriving the nucleic acid molecule from a vector known to include the same, or by isolating directly from cells and tissues containing the same, using standard techniques. Alternatively, the nucleic acid of interest can be produced synthetically, rather than cloned.

A number of viral based systems have been developed for gene transfer into mammalian cells. For example, retroviruses provide a convenient platform for gene delivery systems. A selected gene can be inserted into a vector and packaged in retroviral particles using techniques known in the art. The recombinant virus can then be isolated and delivered to cells of the subject either in vivo or ex vivo. A number of retroviral systems are known in the art. In some embodiments, adenovirus vectors are used. A number of adenovirus vectors are known in the art. In one embodiment, lentivirus vectors are used.

In brief summary, the expression of natural or synthetic nucleic acids encoding CARs is typically achieved by operably linking a nucleic acid encoding the CAR polypeptide or portions thereof to a promoter, and incorporating the construct into an expression vector. The vectors can be suitable for replication and integration eukaryotes. Typical cloning vectors contain transcription and translation terminators, initiation sequences, and promoters useful for regulation of the expression of the desired nucleic acid sequence.

### 7.3.6. Administration of CAR Molecules

CAR molecules are typically administered as a population of effector cells, for example a population of T cells, engineered to express a CD19 CAR molecule.

The effector cell can be transformed with the CAR such that the CAR molecule is expressed on the cell surface. Suitable CAR molecules are described in Section 7.3.4. Populations of cells, e.g., immune effector cells, that express are CAR molecule are referred to herein as “CAR compositions”. CAR compositions can be administered to a subject parenterally, most preferably as an infusion. The cells may be administered as a single infusion or in multiple infusions over a range of time.

In some embodiments, the cell (e.g., T cell) is transduced with a viral vector encoding a CAR. Suitable viral vectors are retroviral vectors and lentiviral vectors In some such embodiments, the cell may stably express the CAR.

In other embodiments, the cell (e.g., T cell) is transfected with a nucleic acid, e.g., mRNA, cDNA, DNA, encoding a CAR. In some such embodiments, the cell may transiently express the CAR.

In certain aspects, the CAR composition comprises a CAR molecule having the sequence of SEQ ID NO:149 or SEQ ID NO:97. In certain aspects, the CAR composition comprises a CAR molecule having the sequence of SEQ ID NO:1162 or SEQ ID NO:1160.

In certain aspects, the CAR composition comprises CTL019.

In certain aspect, the CAR composition has the USAN or INN designation tisagenlecleucel. Tisagenlecleucel is marketed as KYMRIAH®. See, e.g., KYMRIAH® prescribing information, available at www.pharma.us.novartis.com/sites/www.pharma.us.novartis.com/files/kymriah.pdf.

In other aspects, the CAR composition has the USAN or INN designation axicabtagene ciloleucel. Axicabtagene ciloleucel is marketed as YESCARTA®. See, e.g., YESCARTA® prescribing information, available at www.yescarta.com/files/yescarta-pi.pdf. In other aspects, the CAR composition has the USAN designation brexucabtagene autoleucel. Brexucabtagene autoleucel is marketed as TECARTUS™. See, e.g., TECARTUS™ prescribing information, available at www.gilead.com/-/media/files/pdfs/medicines/oncology/tecartus/tecartus-pi.pdf. In other aspects, the CAR composition has the USAN or INN designation lisocabtagene maraleucel. Lisocabtagene maraleucel is marketed as BREYANZI®. See, e.g., BREYANZI® prescribing information, available at packageinserts.bms.com/pi/pi_breyanzi.pdf.

### 7.4. B Cell Targeting Agents

The combinations of the disclosure include a B cell targeting agent. Exemplary B cell targeting agents include BAFFR binding molecules, CD20 binding molecules, CD22 binding molecules, and BAFF binding molecules.

### 7.4.1. BAFFR Binding Molecules

In some embodiment, the B cell targeting agent is a BAFFR binding molecule, for example, a BAFFR antibody. Antibodies against BAFFR (“anti-BAFFR antibodies”) are known from e.g. WO 2010/007082 and include antibodies which are characterized by comprising a VH domain with the amino acid sequence of SEQ ID NO: 59 and a VL domain with the amino acid sequence of SEQ ID NO: 60. The antibody MOR6654 is one such antibody (IgG1 kappa). It has the heavy chain amino acid sequence of SEQ ID NO: 61 and the light chain amino acid sequence of SEQ ID NO: 62. This antibody may be expressed from SEQ ID NOs: 249 and 250, preferably in a host cell which lacks fucosyl-transferase, for example in a mammalian cell line with an inactive FUT8 gene (e.g. FUT8^{-/-}), to provide a functional non-fucosylated anti-BAFFR antibody with enhanced ADCC. This antibody is referred to hereafter as MOR6654B or VAY736, or under its international non-proprietary name ianalumab. Alternative ways to produce non-fucosylated antibodies are known in the art.

TABLE 18
ianalumab sequences

| | | SEQ ID |
|---|---|---|
| Portion | Amino acid or Nucleotide Sequence | NO: |
| CDR-H1 | GDSVSSNSAAWG | 53 |
| CDR-H2 | RIYYRSKWYNSYAVSVKS | 54 |
| CDR-H3 | YDWVPKIGVFDS | 55 |
| CDR-L1 | RASQFISSSYLS | 56 |
| CDR-L2 | GSSSRAT | 57 |
| CDR-L3 | QQLYSSPMT | 58 |
| VH | QVQLQQSGPGLVKPSQTLSLTCAISGDSVSSNSAAWGWIRQSPGRGLEWL | 59 |
| | GRIYYRSKWYNSYAVSVKSRITINPDTSKNQFSLQLNSVTPEDTAVYYCARY | |
| | DWVPKIGVFDSWGQGTLVTVSS | |
| VL | DIVLTQSPATLSLSPGERATLSCRASQFISSSYLSWYQQKPGQAPRLLIYGS | 60 |
| | SSRATGVPARFSGSGSGTDFTLTISSLEPEDFAVYYCQQLYSSPMTFGQGT | |
| | KVEIK | |
| Heavy | QVQLQQSGPGLVKPSQTLSLTCAISGDSVSSNSAAWGWIRQSPGRGLEWL | 61 |
| Chain | GRIYYRSKWYNSYAVSVKSRITINPDTSKNQFSLQLNSVTPEDTAVYYCARY | |
| | DWVPKIGVFDSWGQGTLVTVSSASTKGPSVFPLAPSSKSTSGGTAALGCLV | |
| | KDYFPEPVTVSWNSGALTSGVHTFPAVLQSSGLYSLSSVVTVPSSSLGTQT | |
| | YICNVNHKPSNTKVDKRVEPKSCDKTHTCPPCPAPELLGGPSVFLFPPKPK | |
| | DTLMISRTPEVTCVVVDVSHEDPEVKFNWYVDGVEVHNAKTKPREEQYNST | |
| | YRVVSVLTVLHQDWLNGKEYKCKVSNKALPAPIEKTISKAKGQPREPQVYTL | |
| | PPSREEMTKNQVSLTCLVKGFYPSDIAVEWESNGQPENNYKTTPPVLDSDG | |
| | SFFLYSKLTVDKSRWQQGNVFSCSVMHEALHNHYTQKSLSLSPGK | |
| Light Chain | DIVLTQSPATLSLSPGERATLSCRASQFISSSYLSWYQQKPGQAPRLLIYGS | 62 |
| | SSRATGVPARFSGSGSGTDFTLTISSLEPEDFAVYYCQQLYSSPMTFGQGT | |
| | KVEIKRTVAAPSVFIFPPSDEQLKSGTASVVCLLNNFYPREAKVQWKVDNAL | |
| | QSGNSQESVTEQDSKDSTYSLSSTLTLSKADYEKHKVYACEVTHQGLSSPV | |
| | TKSFNRGEC | |
| Full length | ATGGCCTGGGTGTGGACCCTGCCCTTCCTGATGGCCGCTGCCCAGTCA | 249 |
| nucleotide | GTGCAGGCCCAGGTGCAGCTGCAGCAGAGCGGCCCAGGCCTGGTCAA | |
| sequence | GCCCTCTCAGACCCTGTCACTGACCTGCGCCATTTCAGGCGACAGCGTG | |
| (including | AGCAGCAACAGCGCCGCCTGGGGCTGGATCAGGCAGAGCCCCGGTAG | |
| leader | GGGCCTGGAATGGCTGGGCAGGATCTACTACAGGTCCAAGTGGTACAA | |
| sequence | CAGCTACGCCGTGAGCGTGAAGAGCAGGATCACCATCAACCCTGACAC | |
| and | CAGCAAGAACCAGTTCTCACTGCAGCTCAACAGCGTGACCCCCGAGGA | |
| constant | CACCGCCGTGTACTACTGCGCCAGATACGACTGGGTGCCCAAGATCGG | |
| part) of | CGTGTTCGACAGCTGGGGCCAGGGCACCCTGGTGACCGTGTCAAGCGC | |
| heavy | CAGCACCAAGGGCCCCAGCGTGTTCCCCCTGGCCCCCAGCAGCAAGAG | |
| chain; nt 1- | CACCAGCGGCGGCACAGCCGCCCTGGGCTGCCTGGTGAAGGACTACTT | |
| 57 = | CCCCGAGCCCGTGACCGTGTCCTGGAACAGCGGAGCCCTGACCTCCGG | |
| leader; nt | CGTGCACACCTTCCCCGCCGTGCTGCAGAGCAGCGGCCTGTACAGCCT | |
| 58-429 = | GTCCAGCGTGGTGACAGTGCCCAGCAGCAGCCTGGGCACCCAGACCTA | |
| VH; nt 430- | CATCTGCAACGTGAACCACAAGCCCAGCAACACCAAGGTGGACAAGAGA | |
| 1419 = | GTGGAGCCCAAGAGCTGCGACAAGACCCACACCTGCCCCCCCTGCCCA | |
| constant | GCCCCAGAGCTGCTGGGCGGACCCTCCGTGTTCCTGTTCCCCCCCAAG | |
| region | CCCAAGGACACCCTGATGATCAGCAGGACCCCCGAGGTGACCTGCGTG | |
| (hlgG1) | GTGGTGGACGTGAGCCACGAGGACCCAGAGGTGAAGTTCAACTGGTAC | |
| | GTGGACGGCGTGGAGGTGCACAACGCCAAGACCAAGCCCAGAGAGGA | |
| | GCAGTACAACAGCACCTACAGGGTGGTGTCCGTGCTGACCGTGCTGCA | |
| | CCAGGACTGGCTGAACGGCAAGGAATACAAGTGCAAGGTCTCCAACAA | |
| | GGCCCTGCCAGCCCCCATCGAAAAGACCATCAGCAAGGCCAAGGGCCA | |
| | GCCACGGGAGCCCCAGGTGTACACCCTGCCCCCCTCCCGGGAGGAGAT | |
| | GACCAAGAACCAGGTGTCCCTGACCTGTCTGGTGAAGGGCTTCTACCCC | |
| | AGCGACATCGCCGTGGAGTGGGAGAGCAACGGCCAGCCCGAGAACAAC | |
| | TACAAGACCACCCCCCCAGTGCTGGACAGCGACGGCAGCTTCTTCCTGT | |
| | ACAGCAAGCTGACCGTGGACAAGTCCAGGTGGCAGCAGGGCAACGTGT | |
| | TCAGCTGCAGCGTGATGCACGAGGCCCTGCACAACCACTACACCCAGA | |
| | AGAGCCTGAGCCTGTCCCCCGGCAAG | |
| Full length | ATGAGCGTGCTGACCCAGGTGCTGGCTCTGCTGCTGCTGTGGCTGACC | 250 |
| nucleotide | GGCACCAGATGCGATATCGTGCTGACACAGAGCCCCGCCACCCTGAGC | |
| sequence | CTGAGCCCAGGCGAGAGGGCCACCCTGTCCTGCAGGGCCAGCCAGTTT | |
| (including | ATCAGCAGCAGCTACCTGTCCTGGTATCAGCAGAAGCCCGGCCAGGCC | |
| leader | CCTAGACTGCTGATCTACGGCAGCTCCTCTCGGGCCACCGGCGTGCCC | |
| sequence | GCCAGGTTCAGCGGCAGCGGCTCCGGCACCGACTTCACCCTGACAATC | |
| and | AGCAGCCTGGAGCCCGAGGACTTCGCCGTGTACTACTGCCAGCAGCTG | |
| constant | TACAGCTCACCCATGACCTTCGGCCAGGGCACCAAGGTGGAGATCAAG | |
| part) of light | CGTACGGTGGCCGCTCCCAGCGTGTTCATCTTCCCCCCCAGCGACGAG | |
| chain; nt 1- | CAGCTGAAGAGCGGCACCGCCAGCGTGGTGTGCCTGCTGAACAACTTC | |
| 60 = | TACCCCCGGGAGGCCAAGGTGCAGTGGAAGGTGGACAACGCCCTGCAG | |
| leader; nt | AGCGGCAACAGCCAGGAGAGCGTCACCGAGCAGGACAGCAAGGACTCC | |
| 61-384 | ACCTACAGCCTGAGCAGCACCCTGACCCTGAGCAAGGCCGACTACGAG | |
| VL; nt 385- | AAGCATAAGGTGTACGCCTGCGAGGTGACCCACCAGGGCCTGTCCAGC | |
| 705 = | CCCGTGACCAAGAGCTTCAACAGGGGCGAGTGC | |
| constant | | |
| region | | |
| (hkappa) | | |

Table 19 lists CDR, VH, and VL sequences of further exemplary BAFF binders that can be used in the methods and combinations of the disclosure.

TABLE 19
BAFFR Binder Sequences

| Binder | Portion | Sequence | SEQ ID NO: |
|---|---|---|---|
| BAFFR-1 | CDR-H1 | GDSVSSNSAAWG | 53 |
| | CDR-H2 | RIYYRSKWYNSYAVSV | 224 |
| | CDR-H3 | YDWVPKIGVFDS | 55 |
| | VH | QVQLQQSGPGLVKPSQTLSLTCAISGDSVSSNSAAWGWIRQSP | 59 |
| | | GRGLEWLGRIYYRSKWYNSYAVSVKSRITINPDTSKNQFSLQLN | |
| | | SVTPEDTAVYYCARYDWVPKIGVFDSWGQGTLVTVSS | |
| | CDR-L1 | RASQMIDLRYLS | 225 |
| | CDR-L2 | LLIYGSSSRAT | 226 |
| | CDR-L3 | QQLYSSPM | 227 |
| | VL | DIVLTQSPATLSLSPGERATLSCRASQMIDLRYLSWYQQKPGQA | 228 |
| | | PRLLIYGSSSRATGVPARFSGSGSGTDFTLTISSLEPEDFAVYYC | |
| | | QQLYSSPMTFGQGTKVEIKRT | |
| BAFFR-2 | CDR-H1 | GDSVSSNSAAWG | 53 |
| | CDR-H2 | RIYYRSKWYNSYAVSV | 224 |
| | CDR-H3 | YDWVPKIGVFDS | 55 |
| | VH | QVQLQQSGPGLVKPSQTLSLTCAISGDSVSSNSAAWGWIRQSP | 59 |
| | | GRGLEWLGRIYYRSKWYNSYAVSVKSRITINPDTSKNQFSLQLN | |
| | | SVTPEDTAVYYCARYDWVPKIGVFDSWGQGTLVTVSS | |
| | CDR-L1 | RASQFISSSYLS | 56 |
| | CDR-L2 | LLIYGSSSRAT | 226 |
| | CDR-L3 | QQLYSSPM | 227 |
| | VL | DIVLTQSPATLSLSPGERATLSCRASQFISSSYLSWYQQKPGQA | 1338 |
| | | PRLLIYGSSSRATGVPARFSGSGSGTDFTLTISSLEPEDFAVYYC | |
| | | QQLYSSPMTFGQGTKVEIKRT | |
| BAFFR-3 | CDR-H1 | GDSVSSNSAAWG | 53 |
| | CDR-H2 | RIYYRSKWYNNYAVSV | 229 |
| | CDR-H3 | YKWVPKIGVFDS | 230 |
| | VH | QVQLQQSGPGLVKPSQTLSLTCAISGDSVSSNSAAWGWIRQSP | 231 |
| | | GRGLEWLGRIYYRSKWYNNYAVSVKSRITINPDTSKNQFSLQLN | |
| | | SVTPEDTAVYYCARYKWVPKIGVFDSWGQGTLVTVSS | |
| | CDR-L1 | RASQMIDLRYLS | 225 |
| | CDR-L2 | LLIYGSSSRAT | 226 |
| | CDR-L3 | QQFYSSPL | 232 |
| | VL | DIVLTQSPATLSLSPGERATLSCRASQMIDLRYLSWYQQKPGQA | 233 |
| | | PRLLIYGSSSRATGVPARFSGSGSGTDFTLTISSLEPEDFAVYYC | |
| | | QQFYSSPLTFGQGTKVEIKRT | |
| BAFFR-4 | CDR-H1 | GDSVSSNSAAWG | 53 |
| | CDR-H2 | RIYYRSKWYNSYAVSV | 224 |
| | CDR-H3 | YQWVPKIGVFDS | 234 |
| | VH | QVQLQQSGPGLVKPSQTLSLTCAISGDSVSSNSAAWGWIRQSP | 235 |
| | | GRGLEWLGRIYYRSKWYNSYAVSVKSRITINPDTSKNQFSLQLN | |
| | | SVTPEDTAVYYCARYQWVPKIGVFDSWGQGTLVTVSS | |
| | CDR-L1 | RASQEILPEYLS | 236 |
| | CDR-L2 | LLIYGSSSRAT | 226 |
| | CDR-L3 | QQFYSSPL | 232 |
| | VL | DIVLTQSPATLSLSPGERATLSCRASQEILPEYLSWYQQKPGQA | 237 |
| | | PRLLIYGSSSRATGVPARFSGSGSGTDFTLTISSLEPEDFAVYYC | |
| | | QQFYSSPLTFGQGTKVEIKRT | |
| BAFFR-5 | CDR-H1 | GDSVSSNSAAWG | 53 |
| | CDR-H2 | RIYYRSKWYNSYAVSV | 224 |
| | CDR-H3 | YDWVPKIGVFDL | 238 |
| | VH | QVQLQQSGPGLVKPSQTLSLTCAISGDSVSSNSAAWGWIRQSP | 239 |
| | | GRGLEWLGRIYYRSKWYNSYAVSVKSRITINPDTSKNQFSLQLN | |
| | | SVTPEDTAVYYCARYDWVPKIGVFDLWGQGTLVTVSS | |
| | CDR-L1 | RASQWIEAGYLS | 240 |
| | CDR-L2 | LLIYGSSSRAT | 226 |
| | CDR-L3 | QQLYSSPM | 227 |
| | VL | DIVLTQSPATLSLSPGERATLSCRASQWIEAGYLSWYQQKPGQA | 241 |
| | | PRLLIYGSSSRATGVPARFSGSGSGTDFTLTISSLEPEDFAVYYC | |
| | | QQLYSSPMTFGQGTKVEIKRT | |
| BAFFR-6 | CDR-H1 | GDSVSSNSAAWG | 53 |
| | CDR-H2 | RIYYRSKWWNDYAVSV | 242 |
| | CDR-H3 | YDWVPKIGVFDG | 243 |
| | VH | QVQLQQSGPGLVKPSQTLSLTCAISGDSVSSNSAAWGWIRQSP | 244 |
| | | GRGLEWLGRIYYRSKWWNDYAVSVKSRITINPDTSKNQFSLQLN | |
| | | SVTPEDTAVYYCARYDWVPKIGVFDGWGQGTLVTVSS | |
| | CDR-L1 | RASQMIDLRYLS | 225 |
| | CDR-L2 | LLIYGSSSRAT | 226 |
| | CDR-L3 | QQFYSSPL | 232 |
| | VL | DIVLTQSPATLSLSPGERATLSCRASQMIDLRYLSWYQQKPGQA | 233 |
| | | PRLLIYGSSSRATGVPARFSGSGSGTDFTLTISSLEPEDFAVYYC | |
| | | QQFYSSPLTFGQGTKVEIKRT | |
| BAFFR-7 | CDR-H1 | GDSVSSNSAAWG | 53 |
| | CDR-H2 | RIYYRSKWYNDYAVSVKS | 245 |
| | CDR-H3 | YDWVPKIGVFDS | 55 |
| | VH | QVQLQQSGPGLVKPSQTLSLTCAISGDSVSSNSAAWGWIRQSP | 246 |
| | | GRGLEWLGRIYYRSKWYNDYAVSVKSRITINPDTSKNQFSLQLN | |
| | | SVTPEDTAVYYCARYDWVPKIGVFDSWGQGTLVTVSS | |
| | CDR-L1 | RASQFISSSYLS | 56 |
| | CDR-L2 | LLIYGSSSRAT | 226 |
| | CDR-L3 | QQVYDIPIT | 247 |
| | VL | DIVLTQSPATLSLSPGERATLSCRASQFISSSYLSWYQQKPGQA | 248 |
| | | PRLLIYGSSSRATGVPARFSGSGSGTDFTLTISSLEPEDFAVYYC | |
| | | QQVYDIPITFGQGTKVEIKRT | |

In certain aspects, a BAFFR binding molecule comprises heavy chain and light chain CDRs having the amino acid sequences of any one of BAFFR-1 to BAFFR-7 as set forth in Table 19. In a specific embodiment, a BAFFR binding molecule comprises the heavy and light chain CDRs of BAFFR-1 as set forth in Table 19. In a specific embodiment, a BAFFR binding molecule comprises the heavy and light chain CDRs of BAFFR-2 as set forth in Table 19. In a specific embodiment, a BAFFR binding molecule comprises the heavy and light chain CDRs of BAFFR-3 as set forth in Table 19. In a specific embodiment, a BAFFR binding molecule comprises the heavy and light chain CDRs of BAFFR-4 as set forth in Table 19. In a specific embodiment, a BAFFR binding molecule comprises the heavy and light chain CDRs of BAFFR-5 as set forth in Table 19. In a specific embodiment, a BAFFR binding molecule comprises the heavy and light chain CDRs of BAFFR-6 as set forth in Table 19. In a specific embodiment, a BAFFR binding molecule comprises the heavy and light chain CDRs of BAFFR-7 as set forth in Table 19.

In certain embodiments, a BAFFR binding molecule comprises a heavy chain variable region and a light chain variable region having the VH and VL amino acid sequences of BAFFR-1 as set forth in Table 19. In certain embodiments, a BAFFR binding molecule comprises a heavy chain variable region and a light chain variable region having the VH and VL amino acid sequences of BAFFR-2 as set forth in Table 19. In certain embodiments, a BAFFR binding molecule comprises a heavy chain variable region and a light chain variable region having the VH and VL amino acid sequences of BAFFR-3 as set forth in Table 19. In certain embodiments, a BAFFR binding molecule comprises a heavy chain variable region and a light chain variable region having the VH and VL amino acid sequences of BAFFR-4 as set forth in Table 19. In certain embodiments, a BAFFR binding molecule comprises a heavy chain variable region and a light chain variable region having the VH and VL amino acid sequences of BAFFR-5 as set forth in Table 19. In certain embodiments, a BAFFR binding molecule comprises a heavy chain variable region and a light chain variable region having the VH and VL amino acid sequences of BAFFR-6 as set forth in Table 19. In certain embodiments, a BAFFR binding molecule comprises a heavy chain variable region and a light chain variable region having the VH and VL amino acid sequences of BAFFR-7 as set forth in Table 19.

Additional exemplary BAFFR binding molecules are described in WO 2017/214170.

### 7.4.2. CD20 Binding Molecules

In certain aspects, the B cell targeting agent is a CD20 binding molecule, e.g., an anti-CD20 antibody. Various CD20 binding molecules are described in the art, for example in U.S. Pat. Nos. 7,422,739 and 7,850,962, WO 2005/103081, WO 2011/100403, WO 2017/185949, and WO 2018/044172. See also, Du et al., 2017, Auto Immun Highlights. 8(1):12. Exemplary CD20 binding molecules that can be used in the methods and combinations of the disclosure include rituximab, ofatumumab, ocrelizumab, veltuzumab, and obinutuzumab. In some embodiments, the CD20 binding molecule is rituximab. In other embodiments, the CD20 binding molecule is ofatumumab. In other embodiments, the CD20 binding molecule is ocrelizumab. In other embodiments, the CD20 binding molecule is veltuzumab. In other embodiments, the CD20 binding molecule is obinutuzumab.

### 7.4.3. CD22 Binding Molecules

In certain aspects, the B cell targeting agent is a CD22 binding molecule, e.g., an anti-CD22 antibody. Various CD22 binding molecules are described in the art, for example in WO 2009/124109, WO 2017/009476, and WO 2020/185763. See also, Haso et al., 2013, Blood, 121(7): 1165-1174; Wayne et al., 2010, Clin Cancer Res 16(6):1894-1903; Kato et al., 2013, Leuk Res 37(1):83-88. Exemplary CD22 binding molecules that can be used in the methods and combinations of the disclosure include epratuzumab, inotuzumab, and inotuzumab ozogamicin.

### 7.4.4. BAFF Binding Molecules

In certain aspects, the B cell targeting agent is a BAFF binding molecule, e.g., an anti-BAFF antibody. Various anti-BAFF binding molecules are described in the art, for example in WO 2006/025345 and WO 2016/039801. Exemplary BAFF binding molecules that can be used in the methods and combinations of the disclosure include belimumab, tibulizumab, BR3-Fc, blisibimod and atacicept.

In some embodiments, the BAFF binding molecule is belimumab. In other embodiments, the BAFF binding molecule is tibulizumab. In other embodiments, the BAFF binding molecule is BR3-Fc. In other embodiments, the BAFF binding molecule is blisibimod. In other embodiments, the BAFF binding molecule is atacicept.

### 7.5. Pharmaceutical Compositions and Combination Administration

The anti-CD19 agents and B cell targeting agents can be formulated as pharmaceutical compositions containing one or more pharmaceutically acceptable excipients or carriers. To prepare pharmaceutical or sterile compositions, an anti-CD19 agent or B cell targeting agent preparation can be combined with one or more pharmaceutically acceptable excipients and/or carriers. The anti-CD19 agent and B cell targeting agent of a combination are typically formulated as separate pharmaceutical compositions. Each can be provided, for example, in a single dose or multiple dose container.

For example, formulations of anti-CD19 agents and B cell targeting agents can be prepared by mixing the agents with physiologically acceptable carriers, excipients, or stabilizers in the form of, e.g., lyophilized powders, slurries, aqueous solutions, lotions, or suspensions (see, e.g., Hardman et al., 2001, Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

Selecting an administration regimen for an agent depends on several factors, including the serum or tissue turnover rate of the agent, the level of symptoms, the immunogenicity of the agent, and the accessibility of the target cells. In certain embodiments, an administration regimen maximizes the amount of agent or agents delivered to the subject consistent with an acceptable level of side effects. Accordingly, the amount of an anti-CD19 agent and B cell targeting agent delivered depends in part on the particular agents and the severity of the condition being treated. Guidance in selecting appropriate doses of antibodies and small molecules are available (see, e.g., Wawrzynczak, 1996, Antibody Therapy, Bios Scientific Pub. Ltd, Oxfordshire, UK; Kresina (ed.), 1991, Monoclonal Antibodies, Cytokines and Arthritis, Marcel Dekker, New York, N.Y.; Bach (ed.), 1993, Monoclonal Antibodies and Peptide Therapy in Autoimmune Diseases, Marcel Dekker, New York, N.Y.; Baert et al., 2003, New Engl. J. Med. 348:601-608; Milgrom et al., 1999, New Engl. J. Med. 341:1966-1973; Slamon et al., 2001, New Engl. J. Med. 344:783-792; Beniaminovitz et al., 2000, New Engl. J. Med. 342:613-619; Ghosh et al., 2003, New Engl. J. Med. 348:24-32; Lipsky et al., 2000, New Engl. J. Med. 343:1594-1602).

Determination of the appropriate dose is made by the clinician, e.g., using parameters or factors known or suspected in the art to affect treatment or predicted to affect treatment. Generally, a dose begins with an amount somewhat less than the optimum dose and it is increased by small increments thereafter until the desired or optimum effect is achieved relative to any negative side effects. Important diagnostic measures include those of symptoms of, e.g., the inflammation or level of inflammatory cytokines produced.

Actual dosage levels of an anti-CD19 agent or B cell targeting agent in a pharmaceutical composition can be varied so as to obtain an amount of the agent which in combination with another agent is effective to achieve the desired therapeutic response for a particular subject, compositions, and modes of administration, without being toxic to the subject. The selected dosage levels will depend upon a variety of pharmacokinetic factors including the activity of the particular agents, the route of administration, the time of administration, the rate of excretion of the particular agents being employed, the duration of the treatment, other agents (e.g., active agents such as therapeutic drugs or compounds and/or inert materials used as carriers) in combination with the particular anti-CD19 agents and B cell targeting agents employed, the age, sex, weight, condition, general health and prior medical history of the subject being treated, and like factors known in the medical arts.

Compositions comprising CD19 binding molecules and/or B cell targeting agents can be provided, for example, by continuous infusion, or by doses at intervals. Doses can be provided intravenously, subcutaneously, topically, orally, nasally, rectally, intramuscular, intracerebrally, or by inhalation. A specific dose protocol is one involving the maximal dose or dose frequency that avoids significant undesirable side effects.

An effective amount for a particular subject can vary depending on factors such as the condition being treated, the overall health of the subject, the method route and dose of administration and the severity of side effects (see, e.g., Maynard, et al. (1996) A Handbook of SOPs for Good Clinical Practice, Interpharm Press, Boca Raton, Fla.; Dent (2001) Good Laboratory and Good Clinical Practice, Urch Publ., London, UK).

The route of administration for a CD19 binding molecule or B cell targeting agent can be by, e.g., topical or cutaneous application, injection or infusion by intravenous, intraperitoneal, intracerebral, intramuscular, intraocular, intraarterial, intracerebrospinal, intralesional, or by sustained release systems or an implant (see, e.g., Sidman et al., 1983, Biopolymers 22:547-556; Langer et al., 1981, J. Biomed. Mater. Res. 15:167-277; Langer, 1982, Chem. Tech. 12:98-105; Epstein et al., 1985, Proc. Natl. Acad. Sci. USA 82:3688-3692; Hwang et al., 1980, Proc. Natl. Acad. Sci. USA 77:4030-4034; U.S. Pat. Nos. 6,350,466 and 6,316,024). Where necessary, the composition can also include a solubilizing agent and a local anesthetic such as lidocaine to ease pain at the site of the injection. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903.

A composition of the present disclosure can also be administered via one or more routes of administration using one or more of a variety of known methods. As will be appreciated by a skilled artisan, the route and/or mode of administration will vary depending upon the desired results. Selected routes of administration for CD19 binding molecules and B cell targeting agents include intravenous, intramuscular, intradermal, intraperitoneal, subcutaneous, spinal or other general routes of administration, for example by injection or infusion. General administration can represent modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Alternatively, a composition of the disclosure can be administered via a non-general route, such as a topical, epidermal or mucosal route of administration, for example, intranasally, orally, vaginally, rectally, sublingually or topically. In one embodiment, a CD19 binding molecule and/or a B cell targeting agent is administered by infusion. In another embodiment, a CD19 binding molecule and/or B cell targeting agent is administered subcutaneously.

If a CD19 binding molecule and/or a B cell targeting agent is administered in a controlled release or sustained release system, a pump can be used to achieve controlled or sustained release (see Langer, supra; Sefton, 1987, CRC Crit. Ref Biomed. Eng. 14:20; Buchwald et al., 1980, Surgery 88:507; Saudek et al., 1989, N. Engl. J. Med. 321:574). Polymeric materials can be used to achieve controlled or sustained release of the therapies of the disclosure (see, e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 71:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable. A controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more CD19 binding molecules or B cell targeting agents. See, e.g., U.S. Pat. No. 4,526,938, PCT publication WO 91/05548, PCT publication WO 96/20698, Ning et al., 1996, Radiotherapy & Oncology 39:179-189, Song et al., 1995, PDA Journal of Pharmaceutical Science & Technology 50:372-397, Cleek et al., 1997, Pro. Int'l. Symp. Control. Rel. Bioact. Mater. 24:853-854, and Lam et al., 1997, Proc. Int'l. Symp. Control Rel. Bioact. Mater. 24:759-760.

If a CD19 binding molecule and/or a B cell targeting agent is administered topically, it can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995). For non-sprayable topical dosage forms, viscous to semi-solid or solid forms comprising a carrier or one or more excipients compatible with topical application and having a dynamic viscosity, in some instances, greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations where the active ingredient, in some instances, in combination with a solid or liquid inert carrier, is packaged in a mixture with a pressurized volatile (e.g., a gaseous propellant, such as freon) or in a squeeze bottle. Moisturizers or humectants can also be added to pharmaceutical compositions and dosage forms if desired. Examples of such additional ingredients are well-known.

If a composition comprising a CD19 binding molecule or B cell targeting agent is administered intranasally, the CD19 binding molecule or B cell targeting agent can be formulated in an aerosol form, spray, mist or in the form of drops. In particular, prophylactic or therapeutic agents for use according to the present disclosure can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit can be determined by providing a valve to deliver a metered amount. Capsules and cartridges (composed of, e.g., gelatin) for use in an inhaler or insufflator can be formulated containing a powder mix of the CD19 binding molecule or B cell targeting agent and a suitable powder base such as lactose or starch.

In certain embodiments, the CD19 binding molecules and B cell targeting agents can be formulated to ensure proper distribution in vivo. For example, the blood-brain barrier (BBB) excludes many highly hydrophilic compounds. To ensure that the therapeutic compounds of the disclosure cross the BBB (if desired), they can be formulated, for example, in liposomes. For methods of manufacturing liposomes, see, e.g., U.S. Pat. Nos. 4,522,811; 5,374,548; and 5,399,331. The liposomes can comprise one or more moieties which are selectively transported into specific cells or organs, thus enhance targeted drug delivery (see, e.g., Ranade, 1989, J. Clin. Pharmacol. 29:685). Exemplary targeting moieties include folate or biotin (see, e.g., U.S. Pat. No. 5,416,016 to Low et al.); mannosides (Umezawa et al., 1988, Biochem. Biophys. Res. Commun. 153:1038); antibodies (Bloeman et al., 1995, FEBS Lett. 357:140; Owais et al., 1995, Antimicrob. Agents Chemother. 39:180); surfactant protein A receptor (Briscoe et al., 1995, Am. J. Physiol. 1233:134); p 120 (Schreier et al., 1994, J. Biol. Chem. 269:9090); see also Keinanen and Laukkanen, 1994, FEBS Lett. 346:123; Killion and Fidler, 1994, Immunomethods 4:273.

An anti-CD19 agent and a B cell targeting agent combination can be administered to a subject in the same pharmaceutical composition. Alternatively, the anti-CD19 agent and the B cell targeting agent of a combination are administered to a subject in separate pharmaceutical compositions.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. For example, each therapy can be administered to a subject at the same time or sequentially in any order at different points in time; however, if not administered at the same time, they should be administered sufficiently close in time so as to provide the desired therapeutic effect.

An anti-CD19 agent and a B cell targeting agent can be administered simultaneously, in the same or in separate compositions, or sequentially. For sequential administration, the B cell targeting agent can be administered first, and the anti-CD19 agent can be administered second, or the order of administration can be reversed.

The anti-CD19 agent and the B cell targeting agent can be administered to a subject in any appropriate form and by any suitable route. In some embodiments, the routes of administration are the same. In other embodiments the routes of administration are different.

In other embodiments, the delivery of one treatment ends before the delivery of the other treatment begins, e.g., administration of the B cell targeting agent ends before administration of the anti-CD19 agent begins.

In some embodiments, the treatment is more effective because of combined administration. For example, the anti-CD19 agent therapy is more effective, e.g., an equivalent effect is seen with less of the anti-CD19 agent, or the B cell targeting agent reduces CRS symptoms than would be experienced if the anti-CD19 agent were administered in the absence of the B cell targeting agent. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The combinations of the disclosure comprising an anti-CD19 agent and a B cell targeting agent can further comprise one or more additional agents, for example a corticosteroid (e.g., dexamethasone or prednisone) and/or an immunomodulatory imide drug (IMiD) (e.g., lenalidomide, thalidomide, pomalidomide, or iberdomide). In some embodiments, the combination comprises dexamethasone. In some embodiments, the combination comprises lenalidomide. Additional agents are typically formulated in a separate pharmaceutical composition from the anti-CD19 agent and B cell targeting agent.

### 7.6. B Cell Malignancies and Patient Populations

The combinations of the disclosure can be used in the treatment of B cell malignancies. In one aspect, the disclosure provides a method of reducing the severity of one or more symptoms of CRS in a subject having a B cell malignancy and who is to be treated with or is being treated with an anti-CD19 agent, comprising administering a B cell targeting agent to the subject in combination with the anti-CD19 agent.

The present disclosure also provides methods for preventing, treating and/or managing a B cell malignancy associated with CD19-expressing cells (e.g., a hematologic cancer), the methods comprising administering to a subject in need a combination of the disclosure. In one aspect, the subject is a human.

In some embodiments, the B cell malignancy is a hematological cancer.

In some embodiments, the B cell malignancy is a malignant lymphoproliferative condition.

In some embodiments, the B cell malignancy is a plasma cell dyscrasia.

In some embodiments, the B cell malignancy is an acute leukemia. In some embodiments, the B cell malignancy is B cell acute lymphocytic leukemia (also known as B cell acute lymphoblastic leukaemia or B cell acute lymphoid leukemia) (ALL or B-ALL), e.g., relapsed and/or refractory B-ALL.

In some embodiments, the B cell malignancy is a non-Hodgkin's lymphoma (NHL), for example, chronic lymphocytic leukemia (CLL)/small lymphocytic lymphoma (SLL), follicular lymphoma (FL), mantle cell lymphoma (MCL), diffuse large B-cell lymphoma (DLBCL), Burkitt lymphoma, lymphoplasmacytic lymphoma (Waldenstrom macroglobulinemia), MALT lymphoma (mucosa-associated lymphoid tissue lymphoma) marginal zone lymphoma (MZL) (e.g., extranodal marginal zone lymphoma (EMZL) or nodal marginal zone B-cell lymphoma (NZML)).

In some embodiments, the B cell malignancy is a relapsed and/or refractory non-Hodgkin's lymphoma (NHL).

In some embodiments, the B cell malignancy is chronic lymphocytic leukemia (CLL)/small lymphocytic lymphoma (SLL), e.g., relapsed and/or refractory CLL/SLL.

In some embodiments, the B cell malignancy is follicular lymphoma (FL), e.g., relapsed and/or refractory FL. In some embodiments, the FL is small cell FL. In other embodiments, the FL is large cell FL.

In some embodiments, the B cell malignancy is mantle cell lymphoma (MCL), e.g., relapsed and/or refractory MCL.

In some embodiments, the B cell malignancy is diffuse large B-cell lymphoma (DLBCL), e.g., relapsed and/or refractory DLBCL.

In some embodiments, the B cell malignancy is Burkitt lymphoma.

In some embodiments, the B cell malignancy is lymphoplasmacytic lymphoma (Waldenstrom macroglobulinemia).

In some embodiments, the B cell malignancy is MALT lymphoma (mucosa-associated lymphoid tissue lymphoma).

In some embodiments, the B cell malignancy is marginal zone lymphoma (MZL).

In some embodiments, the B cell malignancy is extranodal marginal zone lymphoma (EMZL).

In some embodiments, the B cell malignancy is nodal marginal zone B-cell lymphoma (NZML).

In some embodiments, the B cell malignancy is splenic marginal zone B-cell lymphoma (SMZL).

In some embodiments, the B cell malignancy is a Hodgkin's lymphoma.

In some embodiments, the B cell malignancy is multiple myeloma.

In some embodiments, the B cell malignancy is hairy cell leukemia.

In some embodiments, the B cell malignancy is primary effusion lymphoma.

In some embodiments, the B cell malignancy is B cell prolymphocytic leukemia.

In some embodiments, the B cell malignancy is plasmablastic lymphoma.

In some embodiments, the B cell malignancy is follicle center lymphoma.

In some embodiments, the B cell malignancy is precursor B-lymphoblastic leukemia.

In some embodiments, the B cell malignancy is high-grade B-cell lymphoma.

In some embodiments, the B cell malignancy is primary mediastinal large B-cell lymphoma.

Certain aspects of the foregoing embodiments relate to subjects having an NHL and who (i) have failed at least one prior line (and optionally up to five prior lines) of standard of care therapy, e.g., an anti-CD20 therapy such as rituximab and/or (ii) is intolerant to or ineligible for one or more other approved therapies, e.g., autologous stem cell transplant (ASCT) and/or (iii) is a non-responder to a chimeric antigen receptor (CAR) T cell therapy. The NHL can be chronic lymphocytic leukemia (CLL)/small lymphocytic lymphoma (SLL), follicular lymphoma (FL), mantle cell lymphoma (MCL), diffuse large B-cell lymphoma (DLBCL), Burkitt lymphoma, lymphoplasmacytic lymphoma (Waldenstrom macroglobulinemia), MALT lymphoma (mucosa-associated lymphoid tissue lymphoma) marginal zone lymphoma (MZL) (e.g., extranodal marginal zone lymphoma (EMZL) or nodal marginal zone B-cell lymphoma (NZML)). In some embodiments, the NHL can relapsed and/or refractory, such as relapsed and/or refractory DLBCL or MCL.

Thus, in certain aspects, a subject having an NHL to whom a combination of the disclosure is administered has failed at least one prior line of standard of care therapy and optionally up to five standard of care therapies. In various embodiments, the subject has failed one, two, three, four or five standard of care therapies. Exemplary standard of care therapies for B cell malignancies include anti-CD20 therapies such as rituximab.

In further aspects, a subject having an NHL to whom a combination of the disclosure is administered is intolerant to or ineligible for one or more other approved therapies, e.g., autologous stem cell transplant (ASCT).

In yet further aspects, a subject having an NHL to whom a combination of the disclosure is administered is a non-responder to chimeric antigen receptor (CAR) T cell therapy composition (“CAR composition”), e.g., an anti-CD19 CAR composition. In certain embodiments, the CAR composition comprises CTL019. In other embodiments, the CAR composition has the USAN or INN designation tisagenlecleucel. Tisagenlecleucel is marketed as KYMRIAH®. See, e.g., KYMRIAH® prescribing information, available at www.pharma.us.novartis.com/sites/www.pharma.us.novartis.com/files/kymriah.pdf. In other embodiments, the CAR composition has the USAN or INN designation axicabtagene ciloleucel. Axicabtagene ciloleucel is marketed as YESCARTA®. See, e.g., YESCARTA® prescribing information, available at www.yescarta.com/files/yescarta-pi.pdf. In other aspects, the CAR composition has the USAN designation brexucabtagene autoleucel. Brexucabtagene autoleucel is marketed as TECARTUS™. See, e.g., TECARTUS™ prescribing information, available at www.gilead.com/-/media/files/pdfs/medicines/oncology/tecartus/tecartus-pi.pdf. In yet other embodiments, the CAR composition has the USAN or INN designation lisocabtagene maraleucel. Lisocabtagene maraleucel is marketed as BREYANZI®. See, e.g., BREYANZI® prescribing information, available at packageinserts.bms.com/pi/pi_breyanzi.pdf.

In some embodiments, when a combination of the disclosure is administered is a non-responder to chimeric antigen receptor (CAR) T cell therapy composition (“CAR composition”), the anti-CD19 agent does not comprise a chimeric antigen receptor and/or is not a CAR composition. In other embodiments, however, the anti-CD19 agent may comprise a chimeric antigen receptor and/or be a CAR composition, for example a different CAR composition from that to which the subject did not respond. Thus, the use of an anti-CD19 agent in a CAR format in a combination of the disclosure can be part of an alternative CAR therapy for the subject.

### 8. EXAMPLES

The examples below relate, in part, to the identification of novel CD19 binders, NEG218 and NEG258, that bind to human CD19 and are cross-reactive with cynomolgus (cyno) CD19, their incorporation into bispecific (BSP) and trispecific (TSP) binding molecules that engage CD3 and, in the case of the TSPs, CD2, as well as extensive characterization of the anti-tumor and immunostimulatory activities of the BSPs and TSPs.

In functional assays, the TSPs, particularly CD3hi TSP1, demonstrate enhanced tumor cell killing and T cell activation & proliferation as compared to the corresponding BSPs. While both CD3hi TSP1 and CD3med TSP1 demonstrate effective anti-tumor responses on established tumors in tumor-bearing mice, T-cell activation by CD3hi TSP1 is particularly effective at enriching T cells with a younger and more functional phenotype. Additionally, CD3hi TSP1 is particularly effective in activating CD28neg CD8-T cells, the exhausted/terminally differentiated cytotoxic T cells. Further, CD3hi TSP1-treated T cells better retain ability to kill target cells upon repeated challenges.

Altogether, this evidence presented herein indicates that the use of CD2 co-stimulation, particularly via a CD58 moiety, results in a CD19 binding molecule that can engage T cells in a manner that can achieve optimal T cell activation and prevent exhaustion, potentially resulting in a more effective and durable anti-tumor response.

The TSPs, particularly CD3hi TSP1, are optimized for a combination of factors, ranging from a novel CD19 binding domain that cross-reacts with cyno CD19, the inclusion of a CD2 binding moiety, the nature and affinity of the T-cell binding moieties (CD58 vs. an anti-CD2 antibody, the relatively “high” or “medium” affinity of the CD3 binding moiety), and the configuration of the binding moieties in the molecules (e.g., CD19 at the N-terminus), all of which individually confer advantageous properties that are expected to result in superior CD19 therapeutics.

Ianalumab is a fully human IgG1 (immunoglobulin subclass G1) monoclonal antibody (mAb) which binds with similar potency to BAFF-R expressed on human, cynomolgus monkey and mouse B cells. Examples 7-8 below show that the anti-BAFFR antibody ianalumab is capable of depleting healthy B cells in vivo in both mouse and cynomolgus monkey. It is expected that administering ianalumab to a subject suffering from a B cell malignancy prior to administering an anti-CD19 agent to the subject will reduce the number of healthy B cells in the subject exposed to the anti-CD19 agent, thereby reducing the severity of CRS experienced by the subject compared to the CRS which would be experienced by the subject in the absence of ianalumab administration.

### 8.1. Example 1: Production of Anti-CD3-Anti-CD19 IgG1 Bispecific and Trispecific Binding Molecules in Knob-into-Holes Format

### 8.1.1. Example 1A: Initial BBM and TBM Constructs

BBMs having a CD3 ABM and a CD19 ABM (shown schematically in FIG. 3A), and TBMs having a CD3 ABM, a CD19 ABM, and a CD2 ABM (shown schematically in FIG. 3B) were produced in a knob-into-hole (KIH) format. Each BBM and TBM of this Example comprises a first half antibody (shown schematically as the left half of each construct shown in FIGS. 3A-3B) and a second half antibody (shown schematically as the right half of each construct shown in FIGS. 3A-3B).

### 8.1.1.1. Materials and Methods

### 8.1.1.1.1. Plasmids Encoding BBMs and TBMs

Plasmids for all constructs were synthesized and codon optimized for expression in mammalian cells.

For each bispecific construct, three plasmids were synthesized. A first plasmid encoding an anti-CD19 heavy chain was synthesized as a fusion comprising (in the N-terminal to C-terminal direction) (i) an anti-CD19 VH domain and (ii) a constant hIgG1 domain containing T366S, L368A, and Y407V mutations for a hole to facilitate heterodimerization as well as silencing mutations. A second plasmid encoding a light chain was synthesized as a fusion comprising (in the N-terminal to C-terminal direction) (i) an anti-CD19 VL domain and (ii) a constant human kappa sequence. The proteins encoded by the first and second plasmids form the first half antibody. A third plasmid encoding the second half antibody was synthesized as a fusion comprising (in the N-terminal to C-terminal direction) (i) an anti-CD3 single chain variable fragment (having the VH and VL domains of an anti-CD3 antibody designated as CD3hi (as defined in the following paragraph)), (ii) a linker, and (iii) a constant hIgG1 domain containing a T366W mutation for a knob to facilitate heterodimerization as well as silencing mutations.

For each trispecific construct, three plasmids were synthesized. A first plasmid encoding an anti-CD19 heavy chain was synthesized as a fusion comprising (in the N-terminal to C-terminal direction) (i) an anti-CD19 VH domain fused to a constant hIgG1 CH1 domain, (ii) a linker, (iii) an anti-CD3 scFv with VH and VL domains of an anti-CD3 antibody having high, medium, or low affinity to CD3 (in relative terms), and referred to herein as CD3hi, CD3med or CD3lo (from anti-CD3 antibodies having an affinity to CD3 of 16 nM, 30 nM, or 48 nm, respectively, as measured by Biacore), (iv) a second linker, and (v) an hIgG1 Fc domain containing T366S, L368A, and Y407V mutations for a hole to facilitate heterodimerization as well as silencing mutations. It should be understood that with respect to the mentioned Biacore affinity values and relative terms in the construct names, these are used merely for identification purposes and are not intended to represent absolute affinity values. A second plasmid encoding a light chain was synthesized as a fusion comprising (in the N-terminal to C-terminal direction) (i) an anti-CD19 VL domain and (ii) a constant human kappa sequence. The proteins encoded by the first and second plasmids form the first half antibody. A third plasmid encoding the second half antibody was synthesized as a fusion comprising (in the N-terminal to C-terminal direction) (i) the IgV domain of CD58 (CD58-6) and (ii) a constant hIgG1 domain containing a T366W mutation for a knob to facilitate heterodimerization as well as silencing mutations.

Control constructs corresponding to the CD3hi TSP1 (which has a NEG258-based CD19 binding arm) and CD3hi TSP2 (which has a NEG218-based CD19 binding arm) trispecific constructs were produced in which the CD2 ABM was replaced with a Vhh against hen egg lysozyme (such control constructs having the names CD3hi TSP1L and CD3hi TSP2L, respectively).

Amino acid sequences for components of the constructs are shown in Table 20A-1 (without Fc sequences) and Table 20A-2 (with Fc sequences).

TABLE 20A-1
Amino acid sequences

| | | | SEQ |
|---|---|---|---|
| Construct | Chain | | ID |
| Name | Description | Amino Acid Sequence | NO: |
| CD3hi | First Half | QVQLVQSGAEVKKPGASVKVSCKASGYTFTTYWIQWVRQAPG | 63 |
| TSP1 | Antibody | QRLEWMGAVYPGDADTRYTQKFQGRVTLTADRSASTAYMELS | |
| | Heavy | SLRSEDTAVYYCGRDAGLEYYALDYWGQGTLVTVSSASTKGP | |
| | Chain | SVFPLAPSSKSTSGGTAALGCLVKDYFPEPVTVSWNSGALTSG | |
| | (Fc | VHTFPAVLQSSGLYSLSSVVTVPSSSLGTQTYICNVNHKPSNTK | |
| | sequence | VDKRVEPKSCGGGGSGGGGSEVQLVESGGGLVQPGGSLKLS | |
| | not shown) | CAASGFTFNTYAMNWVRQASGKGLEWVGRIRSKYNNYATYYA | |
| | | DSVKDRFTISRDDSKSTLYLQMNSLKTEDTAVYYCVRHGNFGN | |
| | | SYVSWFAYWGQGTLVTVSSGGGGSGGGGSGGGGSGGGGS | |
| | | QAVVTQEPSLTVSPGGTVTLTCRSSTGAVTTSNYANWVQQKP | |
| | | GQAPRGLIGGTNKRAPWTPARFSGSLLGDKAALTLSGAQPED | |
| | | EAEYFCALWYSNLWVFGGGTKLTVLGGGGS | |
| | First Half | EIVMTQSPATLSVSPGERATLSCRASQDVGTAVAWYQQKPGQ | 64 |
| | Antibody | APRLLIYWASTRHTGIPARFSGSGSGTEFTLTISSLQSEDFAVYF | |
| | Light Chain | CQQYANFPLYTFGQGTKLEIKRTVAAPSVFIFPPSDEQLKSGTA | |
| | | SVVCLLNNFYPREAKVQWKVDNALQSGNSQESVTEQDSKDST | |
| | | YSLSSTLTLSKADYEKHKVYACEVTHQGLSSPVTKSFNRGEC | |
| | Second Half | SQQIYGVVYGNVTFHVPSNVPLKEVLWKKQKDKVAELENSEFR | 65 |
| | Antibody | AFSSFKNRVYLDTVSGSLTIYNLTSSDEDEYEMESPNITDTMKF | |
| | (Fc | FLYVLESGGGGS | |
| | sequence | | |
| | not shown) | | |
| CD3med | First Half | QVQLVQSGAEVKKPGASVKVSCKASGYTFTTYWIQWVRQAPG | 66 |
| TSP1 | Antibody | QRLEWMGAVYPGDADTRYTQKFQGRVTLTADRSASTAYMELS | |
| | Heavy | SLRSEDTAVYYCGRDAGLEYYALDYWGQGTLVTVSSASTKGP | |
| | Chain | SVFPLAPSSKSTSGGTAALGCLVKDYFPEPVTVSWNSGALTSG | |
| | (Fc | VHTFPAVLQSSGLYSLSSWTVPSSSLGTQTYICNVNHKPSNTK | |
| | sequence | VDKRVEPKSCGGGGSGGGGSEVQLVESGGGLVQPGGSLKLS | |
| | not shown) | CAASGFTFNTYAMNWVRQASGKGLEWVGRIRSKYNNYATYYA | |
| | | DSVKDRFTISRDDSKNTAYLQMNSLKTEDTAVYYCVRHGNFGN | |
| | | SYVSWFAHWGQGTLVTVSSGGGGSGGGGSGGGGSGGGGS | |
| | | QAVVTQEPSLTVSPGGTVTLTCGSSTGAVTSSNYANWVQQKP | |
| | | GQAPRGLIGGTNKRAPWTPARFSGSLLGGKAALTLSGAQPED | |
| | | EAEYYCALWYSNLWVFGGGTKLTVLGGGGS | |
| | First Half | EIVMTQSPATLSVSPGERATLSCRASQDVGTAVAWYQQKPGQ | 64 |
| | Antibody | APRLLIYWASTRHTGIPARFSGSGSGTEFTLTISSLQSEDFAVYF | |
| | Light Chain | CQQYANFPLYTFGQGTKLEIKRTVAAPSVFIFPPSDEQLKSGTA | |
| | | SVVCLLNNFYPREAKVQWKVDNALQSGNSQESVTEQDSKDST | |
| | | YSLSSTLTLSKADYEKHKVYACEVTHQGLSSPVTKSFNRGEC | |
| | Second Half | SQQIYGVVYGNVTFHVPSNVPLKEVLWKKQKDKVAELENSEFR | 65 |
| | Antibody | AFSSFKNRVYLDTVSGSLTIYNLTSSDEDEYEMESPNITDTMKF | |
| | (Fc | FLYVLESGGGGS | |
| | sequence | | |
| | not shown) | | |
| CD3lo | First Half | QVQLVQSGAEVKKPGASVKVSCKASGYTFTTYWIQWVRQAPG | 67 |
| TSP1 | Antibody | QRLEWMGAVYPGDADTRYTQKFQGRVTLTADRSASTAYMELS | |
| | | SLRSEDTAVYYCGRDAGLEYYALDYWGQGTLVTVSSASTKGP | |
| | Heavy | SVFPLAPSSKSTSGGTAALGCLVKDYFPEPVTVSWNSGALTSG | |
| | Chain | VHTFPAVLQSSGLYSLSSVVTVPSSSLGTQTYICNVNHKPSNTK | |
| | (Fc | VDKRVEPKSCGGGGSGGGGSEVQLVESGGGLVQPGGSLKLS | |
| | sequence | CAASGFTFNTYAMNWVRQASGKGLEWVGRIRSKYNNYATYYA | |
| | not shown) | DSVKDRFTISRDDSKSTAYLQMNSLKTEDTAVYYCVRHGNFGN | |
| | | SYVSWFAYWGQGTLVTVSSGGGGSGGGGSGGGGSGGGGS | |
| | | QAVVTQEPSLTVSPGGTVTLTCRSSTGAVTTSNYANWVQQKP | |
| | | GQAPRGLIGGTNKRAPWTPARFSGSLLGDKAALTLSGAQPED | |
| | | EAEYFCALWYSNLWVFGGGTKLTVLGGGGS | |
| | First Half | EIVMTQSPATLSVSPGERATLSCRASQDVGTAVAWYQQKPGQ | 64 |
| | Antibody | APRLLIYWASTRHTGIPARFSGSGSGTEFTLTISSLQSEDFAVYF | |
| | Light Chain | CQQYANFPLYTFGQGTKLEIKRTVAAPSVFIFPPSDEQLKSGTA | |
| | | SVVCLLNNFYPREAKVQWKVDNALQSGNSQESVTEQDSKDST | |
| | | YSLSSTLTLSKADYEKHKVYACEVTHQGLSSPVTKSFNRGEC | |
| | Second Half | SQQIYGVVYGNVTFHVPSNVPLKEVLWKKQKDKVAELENSEFR | 65 |
| | Antibody | AFSSFKNRVYLDTVSGSLTIYNLTSSDEDEYEMESPNITDTMKF | |
| | (Fc | FLYVLESGGGGS | |
| | sequence | | |
| | not shown) | | |
| CD3hi | First Half | EVQLVQSGAEVKKPGESLKISCKASGYSFTNYWMNWVRQMP | 68 |
| TSP2 | Antibody | GKGLEWMGMIHPSDSEIRLNQKFQGQVTLSVDKSIGTAYMQW | |
| | Heavy | SSLKASDTAMYYCSRWYYLSSPMDYWGQGTTVTVSSASTKGP | |
| | Chain | SVFPLAPSSKSTSGGTAALGCLVKDYFPEPVTVSWNSGALTSG | |
| | (Fc | VHTFPAVLQSSGLYSLSSVVTVPSSSLGTQTYICNVNHKPSNTK | |
| | sequence | VDKRVEPKSCGGGGSGGGGSEVQLVESGGGLVQPGGSLKLS | |
| | not shown) | CAASGFTFNTYAMNWVRQASGKGLEWVGRIRSKYNNYATYYA | |
| | | DSVKDRFTISRDDSKSTLYLQMNSLKTEDTAVYYCVRHGNFGN | |
| | | SYVSWFAYWGQGTLVTVSSGGGGSGGGGSGGGGSGGGGS | |
| | | QAVVTQEPSLTVSPGGTVTLTCRSSTGAVTTSNYANWVQQKP | |
| | | GQAPRGLIGGTNKRAPWTPARFSGSLLGDKAALTLSGAQPED | |
| | | EAEYFCALWYSNLWVFGGGTKLTVLGGGGS | |
| | First Half | EIVMTQSPATLSVSPGERATLSCRASQDVGTAVAWYQQKPGQ | 69 |
| | Antibody | APRLLIYWASTRHTGIPARFSGSGSGTEFTLTISSLQSEDFAVYF | |
| | Light Chain | CQQYSSYPYTFGQGTKLEIKRTVAAPSVFIFPPSDEQLKSGTAS | |
| | | WVCLLNNFYPREAKVQWKVDNALQSGNSQESVTEQDSKDSTY | |
| | | SLSSTLTLSKADYEKHKVYACEVTHQGLSSPVTKSFNRGEC | |
| | Second Half | SQQIYGVVYGNVTFHVPSNVPLKEVLWKKQKDKVAELENSEFR | 65 |
| | Antibody | AFSSFKNRVYLDTVSGSLTIYNLTSSDEDEYEMESPNITDTMKF | |
| | (Fc | FLYVLESGGGGS | |
| | sequence | | |
| | not shown) | | |
| CD3hi | First Half | QVQLVQSGAEVKKPGASVKVSCKASGYTFTTYWIQWVRQAPG | 63 |
| TSP1L | Antibody | QRLEWMGAVYPGDADTRYTQKFQGRVTLTADRSASTAYMELS | |
| | Heavy | SLRSEDTAVYYCGRDAGLEYYALDYWGQGTLVTVSSASTKGP | |
| | Chain | SVFPLAPSSKSTSGGTAALGCLVKDYFPEPVTVSWNSGALTSG | |
| | (Fc | VHTFPAVLQSSGLYSLSSWVTVPSSSLGTQTYICNVNHKPSNTK | |
| | sequence | VDKRVEPKSCGGGGSGGGGSEVQLVESGGGLVQPGGSLKLS | |
| | not shown) | CAASGFTFNTYAMNWVRQASGKGLEWVGRIRSKYNNYATYYA | |
| | | DSVKDRFTISRDDSKSTLYLQMNSLKTEDTAVYYCVRHGNFGN | |
| | | SYVSWFAYWGQGTLVTVSSGGGGSGGGGSGGGGSGGGGS | |
| | | QAVVTQEPSLTVSPGGTVTLTCRSSTGAVTTSNYANWVQQKP | |
| | | GQAPRGLIGGTNKRAPWTPARFSGSLLGDKAALTLSGAQPED | |
| | | EAEYFCALWYSNLWVFGGGTKLTVLGGGGS | |
| | First Half | EIVMTQSPATLSVSPGERATLSCRASQDVGTAVAWYQQKPGQ | 64 |
| | Antibody | APRLLIYWASTRHTGIPARFSGSGSGTEFTLTISSLQSEDFAVYF | |
| | Light Chain | CQQYANFPLYTFGQGTKLEIKRTVAAPSVFIFPPSDEQLKSGTA | |
| | | SVVCLLNNFYPREAKVQWKVDNALQSGNSQESVTEQDSKDST | |
| | | YSLSSTLTLSKADYEKHKVYACEVTHQGLSSPVTKSFNRGEC | |
| | Second Half | DVQLQASGGGSVQAGGSLRLSCAASGYTIGPYCMGWFRQAP | 70 |
| | Antibody | GKEREGVAAINMGGGITYYADSVKGRFTISQDNAKNTVYLLMN | |
| | (Fc | SLEPEDTAIYYCAADSTIYASYYECGHGLSTGGYGYDSWGQGT | |
| | sequence | QVTVSSGGGGS | |
| | not shown) | | |
| CD3hi | First Half | EVQLVQSGAEVKKPGESLKISCKASGYSFTNYWMNWVRQMP | 68 |
| TSP2L | Antibody | GKGLEWMGMIHPSDSEIRLNQKFQGQVTLSVDKSIGTAYMQW | |
| | Heavy | SSLKASDTAMYYCSRWYYLSSPMDYWGQGTTVTVSSASTKGP | |
| | Chain | SVFPLAPSSKSTSGGTAALGCLVKDYFPEPVTVSWNSGALTSG | |
| | (Fc | VHTFPAVLQSSGLYSLSSVTVPSSSLGTQTYICNVNHKPSNTK | |
| | sequence | VDKRVEPKSCGGGGSGGGGSEVQLVESGGGLVQPGGSLKLS | |
| | not shown) | CAASGFTFNTYAMNWVRQASGKGLEWVGRIRSKYNNYATYYA | |
| | | DSVKDRFTISRDDSKSTLYLQMNSLKTEDTAVYYCVRHGNFGN | |
| | | SYVSWFAYWGQGTLVTVSSGGGGSGGGGSGGGGSGGGGS | |
| | | QAVVTQEPSLTVSPGGTVTLTCRSSTGAVTTSNYANWVQQKP | |
| | | GQAPRGLIGGTNKRAPWTPARFSGSLLGDKAALTLSGAQPED | |
| | | EAEYFCALWYSNLWVFGGGTKLTVLGGGGS | |
| | First Half | EIVMTQSPATLSVSPGERATLSCRASQDVGTAVAWYQQKPGQ | 69 |
| | Antibody | APRLLIYWASTRHTGIPARFSGSGSGTEFTLTISSLQSEDFAVYF | |
| | Light Chain | CQQYSSYPYTFGQGTKLEIKRTVAAPSVFIFPPSDEQLKSGTAS | |
| | | WVCLLNNFYPREAKVQWKVDNALQSGNSQESVTEQDSKDSTY | |
| | | SLSSTLTLSKADYEKHKVYACEVTHQGLSSPVTKSFNRGEC | |
| | Second Half | DVQLQASGGGSVQAGGSLRLSCAASGYTIGPYCMGWFRQAP | 70 |
| | Antibody | GKEREGVAAINMGGGITYYADSVKGRFTISQDNAKNTVYLLMN | |
| | (Fc | SLEPEDTAIYYCAADSTIYASYYECGHGLSTGGYGYDSWGQGT | |
| | sequence | QVTVSSGGGGS | |
| | not shown) | | |
| CD3hi | First Half | QVQLVQSGAEVKKPGASVKVSCKASGYTFTTYWIQWVRQAPG | 71 |
| BSP1-2 | Antibody | QRLEWMGAVYPGDADTRYTQKFQGRVTLTADRSASTAYMELS | |
| arm | Heavy | SLRSEDTAVYYCGRDAGLEYYALDYWGQGTLVTVSSASTKGP | |
| | Chain | SVFPLAPSSKSTSGGTAALGCLVKDYFPEPVTVSWNSGALTSG | |
| | (Fc | VHTFPAVLQSSGLYSLSSVVTVPSSSLGTQTYICNVNHKPSNTK | |
| | sequence | VDKRVEPKSC | |
| | not shown) | | |
| | First Half | EIVMTQSPATLSVSPGERATLSCRASQDVGTAVAWYQQKPGQ | 64 |
| | Antibody | APRLLIYWASTRHTGIPARFSGSGSGTEFTLTISSLQSEDFAVYF | |
| | Light Chain | CQQYANFPLYTFGQGTKLEIKRTVAAPSVFIFPPSDEQLKSGTA | |
| | | SVVCLLNNFYPREAKVQWKVDNALQSGNSQESVTEQDSKDST | |
| | | YSLSSTLTLSKADYEKHKVYACEVTHQGLSSPVTKSFNRGEC | |
| | Second Half | EVQLVESGGGLVQPGGSLKLSCAASGFTFNTYAMNWVRQAS | 72 |
| | Antibody | GKGLEWVGRIRSKYNNYATYYADSVKDRFTISRDDSKSTLYLQ | |
| | | MNSLKTEDTAVYYCVRHGNFGNSYVSWFAYWGQGTLVTVSS | |
| | | GGGGSGGGGSGGGGSGGGGSQAVVTQEPSLTVSPGGTVTLT | |
| | (Fc | CRSSTGAVTTSNYANWVQQKPGQAPRGLIGGTNKRAPWTPA | |
| | sequence | RFSGSLLGDKAALTLSGAQPEDEAEYFCALWYSNLWVFGGGT | |
| | not shown) | KLTVLGGGGS | |
| CD3hi | First Half | EVQLVQSGAEVKKPGESLKISCKASGYSFTNYWMNWVRQMP | 73 |
| BSP2-2 | Antibody | GKGLEWMGMIHPSDSEIRLNQKFQGQVTLSVDKSIGTAYMQW | |
| arm | Heavy | SSLKASDTAMYYCSRWYYLSSPMDYWGQGTTVTVSSASTKGP | |
| | Chain | SVFPLAPSSKSTSGGTAALGCLVKDYFPEPVTVSWNSGALTSG | |
| | (Fc | VHTFPAVLQSSGLYSLSSVTVPSSSLGTQTYICNVNHKPSNTK | |
| | sequence | VDKRVEPKSC | |
| | not shown) | | |
| | First Half | EIVMTQSPATLSVSPGERATLSCRASQDVGTAVAWYQQKPGQ | 69 |
| | Antibody | APRLLIYWASTRHTGIPARFSGSGSGTEFTLTISSLQSEDFAVYF | |
| | Light Chain | CQQYSSYPYTFGQGTKLEIKRTVAAPSVFIFPPSDEQLKSGTAS | |
| | | WVCLLNNFYPREAKVQWKVDNALQSGNSQESVTEQDSKDSTY | |
| | | SLSSTLTLSKADYEKHKVYACEVTHQGLSSPVTKSFNRGEC | |
| | Second Half | EVQLVESGGGLVQPGGSLKLSCAASGFTFNTYAMNWVRQAS | 72 |
| | Antibody | GKGLEWVGRIRSKYNNYATYYADSVKDRFTISRDDSKSTLYLQ | |
| | (Fc | MNSLKTEDTAVYYCVRHGNFGNSYVSWFAYWGQGTLVTVSS | |
| | sequence | GGGGSGGGGSGGGGSGGGGSQAVVTQEPSLTVSPGGTVTLT | |
| | not shown) | CRSSTGAVTTSNYANWVQQKPGQAPRGLIGGTNKRAPWTPA | |
| | | RFSGSLLGDKAALTLSGAQPEDEAEYFCALWYSNLWVFGGGT | |
| | | KLTVLGGGGS | |

Table 20A-2 below shows the full length amino acid sequences of the constructs shown in Table 20A-1, including Fc sequences.

TABLE 20A-2
Amino acid sequences

| | | | SEQ |
|---|---|---|---|
| Construct | Chain | | ID |
| Name | Description | Amino Acid Sequence | NO: |
| CD3hi | First Half | QVQLVQSGAEVKKPGASVKVSCKASGYTFTTYWIQW | 74 |
| TSP1 | Antibody | VRQAPGQRLEWMGAVYPGDADTRYTQKFQGRVTLT | |
| | Heavy Chain | ADRSASTAYMELSSLRSEDTAVYYCGRDAGLEYYAL | |
| | (includes Fc | DYWGQGTLVTVSSASTKGPSVFPLAPSSKSTSGGTA | |
| | sequence) | ALGCLVKDYFPEPVTVSWNSGALTSGVHTFPAVLQS | |
| | | SGLYSLSSVVTVPSSSLGTQTYICNVNHKPSNTKVDK | |
| | | RVEPKSCGGGGSGGGGSEVQLVESGGGLVQPGGSL | |
| | | KLSCAASGFTFNTYAMNWVRQASGKGLEWVGRIRSK | |
| | | YNNYATYYADSVKDRFTISRDDSKSTLYLQMNSLKTE | |
| | | DTAVYYCVRHGNFGNSYVSWFAYWGQGTLVTVSSG | |
| | | GGGSGGGGSGGGGSGGGGSQAVVTQEPSLTVSPG | |
| | | GTVTLTCRSSTGAVTTSNYANWVQQKPGQAPRGLIG | |
| | | GTNKRAPWTPARFSGSLLGDKAALTLSGAQPEDEAE | |
| | | YFCALWYSNLWVFGGGTKLTVLGGGGSDKTHTCPP | |
| | | CPAPELLGGPSVFLFPPKPKDTLMISRTPEVTCVVVA | |
| | | VSHEDPEVKFNWYVDGVEVHNAKTKPREEQYASTYR | |
| | | VVSVLTVLHQDWLNGKEYKCKVSNKALAAPIEKTISKA | |
| | | KGQPREPQVCTLPPSREEMTKNQVSLSCAVKGFYPS | |
| | | DIAVEWESNGQPENNYKTTPPVLDSDGSFFLVSKLTV | |
| | | DKSRWQQGNVFSCSVMHEALHNHYTQKSLSLSPGK | |
| | First Half | EIVMTQSPATLSVSPGERATLSCRASQDVGTAVAWY | 64 |
| | Antibody | QQKPGQAPRLLIYWASTRHTGIPARFSGSGSGTEFTL | |
| | Light Chain | TISSLQSEDFAVYFCQQYANFPLYTFGQGTKLEIKRTV | |
| | | AAPSVFIFPPSDEQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| | Second Half | SQQIYGVVYGNVTFHVPSNVPLKEVLWKKQKDKVAE | 75 |
| | Antibody | LENSEFRAFSSFKNRVYLDTVSGSLTIYNLTSSDEDEY | |
| | (includes Fc | EMESPNITDTMKFFLYVLESGGGGSDKTHTCPPCPA | |
| | sequence) | PELLGGPSVFLFPPKPKDTLMISRTPEVTCVVVAVSH | |
| | | EDPEVKFNWYVDGVEVHNAKTKPREEQYASTYRVVS | |
| | | VLTVLHQDWLNGKEYKCKVSNKALAAPIEKTISKAKG | |
| | | QPREPQVYTLPPCREEMTKNQVSLWCLVKGFYPSDI | |
| | | AVEWESNGQPENNYKTTPPVLDSDGSFFLYSKLTVD | |
| | | KSRWQQGNVFSCSVMHEALHNRYTQKSLSLSPGK | |
| CD3med | First Half | QVQLVQSGAEVKKPGASVKVSCKASGYTFTTYWIQW | 76 |
| TSP1 | Antibody | VRQAPGQRLEWMGAVYPGDADTRYTQKFQGRVTLT | |
| | Heavy Chain | ADRSASTAYMELSSLRSEDTAVYYCGRDAGLEYYAL | |
| | (includes Fc | DYWGQGTLVTVSSASTKGPSVFPLAPSSKSTSGGTA | |
| | sequence) | ALGCLVKDYFPEPVTVSWNSGALTSGVHTFPAVLQS | |
| | | SGLYSLSSVVTVPSSSLGTQTYICNVNHKPSNTKVDK | |
| | | RVEPKSCGGGGSGGGGSEVQLVESGGGLVQPGGSL | |
| | | KLSCAASGFTFNTYAMNWVRQASGKGLEWVGRIRSK | |
| | | YNNYATYYADSVKDRFTISRDDSKNTAYLQMNSLKTE | |
| | | DTAVYYCVRHGNFGNSYVSWFAHWGQGTLVTVSSG | |
| | | GGGSGGGGSGGGGSGGGGSQAVVTQEPSLTVSPG | |
| | | GTVTLTCGSSTGAVTSSNYANWVQQKPGQAPRGLIG | |
| | | GTNKRAPWTPARFSGSLLGGKAALTLSGAQPEDEAE | |
| | | YYCALWYSNLWVFGGGTKLTVLGGGGSDKTHTCPP | |
| | | CPAPELLGGPSVFLFPPKPKDTLMISRTPEVTCVVVA | |
| | | VSHEDPEVKFNWYVDGVEVHNAKTKPREEQYASTYR | |
| | | VVSVLTVLHQDWLNGKEYKCKVSNKALAAPIEKTISKA | |
| | | KGQPREPQVCTLPPSREEMTKNQVSLSCAVKGFYPS | |
| | | DIAVEWESNGQPENNYKTTPPVLDSDGSFFLVSKLTV | |
| | | DKSRWQQGNVFSCSVMHEALHNHYTQKSLSLSPGK | |
| | First Half | EIVMTQSPATLSVSPGERATLSCRASQDVGTAVAWY | 64 |
| | Antibody | QQKPGQAPRLLIYWASTRHTGIPARFSGSGSGTEFTL | |
| | Light Chain | TISSLQSEDFAVYFCQQYANFPLYTFGQGTKLEIKRTV | |
| | | AAPSVFIFPPSDEQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| | Second Half | SQQIYGVVYGNVTFHVPSNVPLKEVLWKKQKDKVAE | 75 |
| | Antibody | LENSEFRAFSSFKNRVYLDTVSGSLTIYNLTSSDEDEY | |
| | (includes Fc | EMESPNITDTMKFFLYVLESGGGGSDKTHTCPPCPA | |
| | sequence) | PELLGGPSVFLFPPKPKDTLMISRTPEVTCVVVAVSH | |
| | | EDPEVKFNWYVDGVEVHNAKTKPREEQYASTYRVVS | |
| | | VLTVLHQDWLNGKEYKCKVSNKALAAPIEKTISKAKG | |
| | | QPREPQVYTLPPCREEMTKNQVSLWCLVKGFYPSDI | |
| | | AVEWESNGQPENNYKTTPPVLDSDGSFFLYSKLTVD | |
| | | KSRWQQGNVFSCSVMHEALHNRYTQKSLSLSPGK | |
| CD3lo | First Half | QVQLVQSGAEVKKPGASVKVSCKASGYTFTTYWIQW | 77 |
| TSP1 | Antibody | VRQAPGQRLEWMGAVYPGDADTRYTQKFQGRVTLT | |
| | Heavy Chain | ADRSASTAYMELSSLRSEDTAVYYCGRDAGLEYYAL | |
| | (includes Fc | DYWGQGTLVTVSSASTKGPSVFPLAPSSKSTSGGTA | |
| | sequence) | ALGCLVKDYFPEPVTVSWNSGALTSGVHTFPAVLQS | |
| | | SGLYSLSSVVTVPSSSLGTQTYICNVNHKPSNTKVDK | |
| | | RVEPKSCGGGGSGGGGSEVQLVESGGGLVQPGGSL | |
| | | KLSCAASGFTFNTYAMNWVRQASGKGLEWVGRIRSK | |
| | | YNNYATYYADSVKDRFTISRDDSKSTAYLQMNSLKTE | |
| | | DTAVYYCVRHGNFGNSYVSWFAYWGQGTLVTVSSG | |
| | | GGGSGGGGSGGGGSGGGGSQAVVTQEPSLTVSPG | |
| | | GTVTLTCRSSTGAVTTSNYANWVQQKPGQAPRGLIG | |
| | | GTNKRAPWTPARFSGSLLGDKAALTLSGAQPEDEAE | |
| | | YFCALWYSNLWVFGGGTKLTVLGGGGSDKTHTCPP | |
| | | CPAPELLGGPSVFLFPPKPKDTLMISRTPEVTCVVVA | |
| | | VSHEDPEVKFNWYVDGVEVHNAKTKPREEQYASTYR | |
| | | VVSVLTVLHQDWLNGKEYKCKVSNKALAAPIEKTISKA | |
| | | KGQPREPQVCTLPPSREEMTKNQVSLSCAVKGFYPS | |
| | | DIAVEWESNGQPENNYKTTPPVLDSDGSFFLVSKLTV | |
| | | DKSRWQQGNVFSCSVMHEALHNHYTQKSLSLSPGK | |
| | First Half | EIVMTQSPATLSVSPGERATLSCRASQDVGTAVAWY | 64 |
| | Antibody | QQKPGQAPRLLIYWASTRHTGIPARFSGSGSGTEFTL | |
| | Light Chain | TISSLQSEDFAVYFCQQYANFPLYTFGQGTKLEIKRTV | |
| | | AAPSVFIFPPSDEQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| | Second Half | SQQIYGVVYGNVTFHVPSNVPLKEVLWKKQKDKVAE | 75 |
| | Antibody | LENSEFRAFSSFKNRVYLDTVSGSLTIYNLTSSDEDEY | |
| | (includes Fc | EMESPNITDTMKFFLYVLESGGGGSDKTHTCPPCPA | |
| | sequence) | PELLGGPSVFLFPPKPKDTLMISRTPEVTCVVVAVSH | |
| | | EDPEVKFNWYVDGVEVHNAKTKPREEQYASTYRVVS | |
| | | VLTVLHQDWLNGKEYKCKVSNKALAAPIEKTISKAKG | |
| | | QPREPQVYTLPPCREEMTKNQVSLWCLVKGFYPSDI | |
| | | AVEWESNGQPENNYKTTPPVLDSDGSFFLYSKLTVD | |
| | | KSRWQQGNVFSCSVMHEALHNRYTQKSLSLSPGK | |
| CD3hi | First Half | EVQLVQSGAEVKKPGESLKISCKASGYSFTNYWMNW | 78 |
| TSP2 | Antibody | VRQMPGKGLEWMGMIHPSDSEIRLNQKFQGQVTLSV | |
| | Heavy Chain | DKSIGTAYMQWSSLKASDTAMYYCSRWYYLSSPMD | |
| | (includes Fc | YWGQGTTVTVSSASTKGPSVFPLAPSSKSTSGGTAA | |
| | sequence) | LGCLVKDYFPEPVTVSWNSGALTSGVHTFPAVLQSS | |
| | | GLYSLSSVVTVPSSSLGTQTYICNVNHKPSNTKVDKR | |
| | | VEPKSCGGGGSGGGGSEVQLVESGGGLVQPGGSLK | |
| | | LSCAASGFTFNTYAMNWVRQASGKGLEWVGRIRSKY | |
| | | NNYATYYADSVKDRFTISRDDSKSTLYLQMNSLKTED | |
| | | TAVYYCVRHGNFGNSYVSWFAYWGQGTLVTVSSGG | |
| | | GGSGGGGSGGGGSGGGGSQAVVTQEPSLTVSPGG | |
| | | TVTLTCRSSTGAVTTSNYANWVQQKPGQAPRGLIGG | |
| | | TNKRAPWTPARFSGSLLGDKAALTLSGAQPEDEAEY | |
| | | FCALWYSNLWVFGGGTKLTVLGGGGSDKTHTCPPC | |
| | | PAPELLGGPSVFLFPPKPKDTLMISRTPEVTCVVVAVS | |
| | | HEDPEVKFNWYVDGVEVHNAKTKPREEQYASTYRVV | |
| | | SVLTVLHQDWLNGKEYKCKVSNKALAAPIEKTISKAK | |
| | | GQPREPQVCTLPPSREEMTKNQVSLSCAVKGFYPSD | |
| | | IAVEWESNGQPENNYKTTPPVLDSDGSFFLVSKLTVD | |
| | | KSRWQQGNVFSCSVMHEALHNHYTQKSLSLSPGK | |
| | First Half | EIVMTQSPATLSVSPGERATLSCRASQDVGTAVAWY | 69 |
| | Antibody | QQKPGQAPRLLIYWASTRHTGIPARFSGSGSGTEFTL | |
| | Light Chain | TISSLQSEDFAVYFCQQYSSYPYTFGQGTKLEIKRTVA | |
| | | APSVFIFPPSDEQLKSGTASVVCLLNNFYPREAKVQW | |
| | | KVDNALQSGNSQESVTEQDSKDSTYSLSSTLTLSKAD | |
| | | YEKHKVYACEVTHQGLSSPVTKSFNRGEC | |
| | Second Half | SQQIYGVVYGNVTFHVPSNVPLKEVLWKKQKDKVAE | 75 |
| | Antibody | LENSEFRAFSSFKNRVYLDTVSGSLTIYNLTSSDEDEY | |
| | (includes Fc | EMESPNITDTMKFFLYVLESGGGGSDKTHTCPPCPA | |
| | sequence) | PELLGGPSVFLFPPKPKDTLMISRTPEVTCVVVAVSH | |
| | | EDPEVKFNWYVDGVEVHNAKTKPREEQYASTYRVVS | |
| | | VLTVLHQDWLNGKEYKCKVSNKALAAPIEKTISKAKG | |
| | | QPREPQVYTLPPCREEMTKNQVSLWCLVKGFYPSDI | |
| | | AVEWESNGQPENNYKTTPPVLDSDGSFFLYSKLTVD | |
| | | KSRWQQGNVFSCSVMHEALHNRYTQKSLSLSPGK | |
| CD3hi | First Half | QVQLVQSGAEVKKPGASVKVSCKASGYTFTTYWIQW | 74 |
| TSP1L | Antibody | VRQAPGQRLEWMGAVYPGDADTRYTQKFQGRVTLT | |
| | Heavy Chain | ADRSASTAYMELSSLRSEDTAVYYCGRDAGLEYYAL | |
| | (includes Fc | DYWGQGTLVTVSSASTKGPSVFPLAPSSKSTSGGTA | |
| | sequence) | ALGCLVKDYFPEPVTVSWNSGALTSGVHTFPAVLQS | |
| | | SGLYSLSSVVTVPSSSLGTQTYICNVNHKPSNTKVDK | |
| | | RVEPKSCGGGGSGGGGSEVQLVESGGGLVQPGGSL | |
| | | KLSCAASGFTFNTYAMNWVRQASGKGLEWVGRIRSK | |
| | | YNNYATYYADSVKDRFTISRDDSKSTLYLQMNSLKTE | |
| | | DTAVYYCVRHGNFGNSYVSWFAYWGQGTLVTVSSG | |
| | | GGGSGGGGSGGGGSGGGGSQAVVTQEPSLTVSPG | |
| | | GTVTLTCRSSTGAVTTSNYANWVQQKPGQAPRGLIG | |
| | | GTNKRAPWTPARFSGSLLGDKAALTLSGAQPEDEAE | |
| | | YFCALWYSNLWVFGGGTKLTVLGGGGSDKTHTCPP | |
| | | CPAPELLGGPSVFLFPPKPKDTLMISRTPEVTCVVVA | |
| | | VSHEDPEVKFNWYVDGVEVHNAKTKPREEQYASTYR | |
| | | VVSVLTVLHQDWLNGKEYKCKVSNKALAAPIEKTISKA | |
| | | KGQPREPQVCTLPPSREEMTKNQVSLSCAVKGFYPS | |
| | | DIAVEWESNGQPENNYKTTPPVLDSDGSFFLVSKLTV | |
| | | DKSRWQQGNVFSCSVMHEALHNHYTQKSLSLSPGK | |
| | First Half | EIVMTQSPATLSVSPGERATLSCRASQDVGTAVAWY | 64 |
| | Antibody | QQKPGQAPRLLIYWASTRHTGIPARFSGSGSGTEFTL | |
| | Light Chain | TISSLQSEDFAVYFCQQYANFPLYTFGQGTKLEIKRTV | |
| | | AAPSVFIFPPSDEQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| | Second Half | DVQLQASGGGSVQAGGSLRLSCAASGYTIGPYCMG | 79 |
| | Antibody | WFRQAPGKEREGVAAINMGGGITYYADSVKGRFTIS | |
| | (includes Fc | QDNAKNTVYLLMNSLEPEDTAIYYCAADSTIYASYYEC | |
| | sequence) | GHGLSTGGYGYDSWGQGTQVTVSSGGGGSDKTHT | |
| | | CPPCPAPELLGGPSVFLFPPKPKDTLMISRTPEVTCV | |
| | | VVAVSHEDPEVKFNWYVDGVEVHNAKTKPREEQYAS | |
| | | TYRVVSVLTVLHQDWLNGKEYKCKVSNKALAAPIEKTI | |
| | | SKAKGQPREPQVYTLPPCREEMTKNQVSLWCLVKGF | |
| | | YPSDIAVEWESNGQPENNYKTTPPVLDSDGSFFLYSK | |
| | | LTVDKSRWQQGNVFSCSVMHEALHNRYTQKSLSLSP | |
| | | GK | |
| CD3hi | First Half | EVQLVQSGAEVKKPGESLKISCKASGYSFTNYWMNW | 78 |
| TSP2L | Antibody | VRQMPGKGLEWMGMIHPSDSEIRLNQKFQGQVTLSV | |
| | Heavy Chain | DKSIGTAYMQWSSLKASDTAMYYCSRWYYLSSPMD | |
| | (includes Fc | YWGQGTTVTVSSASTKGPSVFPLAPSSKSTSGGTAA | |
| | sequence) | LGCLVKDYFPEPVTVSWNSGALTSGVHTFPAVLQSS | |
| | | GLYSLSSVVTVPSSSLGTQTYICNVNHKPSNTKVDKR | |
| | | VEPKSCGGGGSGGGGSEVQLVESGGGLVQPGGSLK | |
| | | LSCAASGFTFNTYAMNWVRQASGKGLEWVGRIRSKY | |
| | | NNYATYYADSVKDRFTISRDDSKSTLYLQMNSLKTED | |
| | | TAVYYCVRHGNFGNSYVSWFAYWGQGTLVTVSSGG | |
| | | GGSGGGGSGGGGSGGGGSQAVVTQEPSLTVSPGG | |
| | | TVTLTCRSSTGAVTTSNYANWVQQKPGQAPRGLIGG | |
| | | TNKRAPWTPARFSGSLLGDKAALTLSGAQPEDEAEY | |
| | | FCALWYSNLWVFGGGTKLTVLGGGGSDKTHTCPPC | |
| | | PAPELLGGPSVFLFPPKPKDTLMISRTPEVTCVVVAVS | |
| | | HEDPEVKFNWYVDGVEVHNAKTKPREEQYASTYRVV | |
| | | SVLTVLHQDWLNGKEYKCKVSNKALAAPIEKTISKAK | |
| | | GQPREPQVCTLPPSREEMTKNQVSLSCAVKGFYPSD | |
| | | IAVEWESNGQPENNYKTTPPVLDSDGSFFLVSKLTVD | |
| | | KSRWQQGNVFSCSVMHEALHNHYTQKSLSLSPGK | |
| | First Half | EIVMTQSPATLSVSPGERATLSCRASQDVGTAVAWY | 69 |
| | Antibody | QQKPGQAPRLLIYWASTRHTGIPARFSGSGSGTEFTL | |
| | Light Chain | TISSLQSEDFAVYFCQQYSSYPYTFGQGTKLEIKRTVA | |
| | | APSVFIFPPSDEQLKSGTASVVCLLNNFYPREAKVQW | |
| | | KVDNALQSGNSQESVTEQDSKDSTYSLSSTLTLSKAD | |
| | | YEKHKVYACEVTHQGLSSPVTKSFNRGEC | |
| | Second Half | DVQLQASGGGSVQAGGSLRLSCAASGYTIGPYCMG | 79 |
| | Antibody | WFRQAPGKEREGVAAINMGGGITYYADSVKGRFTIS | |
| | (includes Fc | QDNAKNTVYLLMNSLEPEDTAIYYCAADSTIYASYYEC | |
| | sequence) | GHGLSTGGYGYDSWGQGTQVTVSSGGGGSDKTHT | |
| | | CPPCPAPELLGGPSVFLFPPKPKDTLMISRTPEVTCV | |
| | | VVAVSHEDPEVKFNWYVDGVEVHNAKTKPREEQYAS | |
| | | TYRVVSVLTVLHQDWLNGKEYKCKVSNKALAAPIEKTI | |
| | | SKAKGQPREPQVYTLPPCREEMTKNQVSLWCLVKGF | |
| | | YPSDIAVEWESNGQPENNYKTTPPVLDSDGSFFLYSK | |
| | | LTVDKSRWQQGNVFSCSVMHEALHNRYTQKSLSLSP | |
| | | GK | |
| CD3hi | First Half | QVQLVQSGAEVKKPGASVKVSCKASGYTFTTYWIQW | 80 |
| BSP1-2 | Antibody | VRQAPGQRLEWMGAVYPGDADTRYTQKFQGRVTLT | |
| arm | Heavy Chain | ADRSASTAYMELSSLRSEDTAVYYCGRDAGLEYYAL | |
| | (includes Fc | DYWGQGTLVTVSSASTKGPSVFPLAPSSKSTSGGTA | |
| | sequence) | ALGCLVKDYFPEPVTVSWNSGALTSGVHTFPAVLQS | |
| | | SGLYSLSSVVTVPSSSLGTQTYICNVNHKPSNTKVDK | |
| | | RVEPKSCDKTHTCPPCPAPELLGGPSVFLFPPKPKDT | |
| | | LMISRTPEVTCVVVAVSHEDPEVKFNWYVDGVEVHN | |
| | | AKTKPREEQYASTYRVVSVLTVLHQDWLNGKEYKCK | |
| | | VSNKALAAPIEKTISKAKGQPREPQVCTLPPSREEMT | |
| | | KNQVSLSCAVKGFYPSDIAVEWESNGQPENNYKTTP | |
| | | PVLDSDGSFFLVSKLTVDKSRWQQGNVFSCSVMHEA | |
| | | LHNHYTQKSLSLSPGK | |
| | First Half | EIVMTQSPATLSVSPGERATLSCRASQDVGTAVAWY | 64 |
| | Antibody | QQKPGQAPRLLIYWASTRHTGIPARFSGSGSGTEFTL | |
| | Light Chain | TISSLQSEDFAVYFCQQYANFPLYTFGQGTKLEIKRTV | |
| | | AAPSVFIFPPSDEQLKSGTASVVCLLNNFYPREAKVQ | |
| | | WKVDNALQSGNSQESVTEQDSKDSTYSLSSTLTLSK | |
| | | ADYEKHKVYACEVTHQGLSSPVTKSFNRGEC | |
| | Second Half | EVQLVESGGGLVQPGGSLKLSCAASGFTFNTYAMN | 81 |
| | Antibody | WVRQASGKGLEWVGRIRSKYNNYATYYADSVKDRFT | |
| | (includes Fc | ISRDDSKSTLYLQMNSLKTEDTAVYYCVRHGNFGNSY | |
| | sequence) | VSWFAYWGQGTLVTVSSGGGGSGGGGSGGGGSG | |
| | | GGGSQAVVTQEPSLTVSPGGTVTLTCRSSTGAVTTS | |
| | | NYANWVQQKPGQAPRGLIGGTNKRAPWTPARFSGS | |
| | | LLGDKAALTLSGAQPEDEAEYFCALWYSNLWVFGGG | |
| | | TKLTVLGGGGSDKTHTCPPCPAPELLGGPSVFLFPPK | |
| | | PKDTLMISRTPEVTCVVVAVSHEDPEVKFNWYVDGV | |
| | | EVHNAKTKPREEQYASTYRVVSVLTVLHQDWLNGKE | |
| | | YKCKVSNKALAAPIEKTISKAKGQPREPQVYTLPPCR | |
| | | EEMTKNQVSLWCLVKGFYPSDIAVEWESNGQPENNY | |
| | | KTTPPVLDSDGSFFLYSKLTVDKSRWQQGNVFSCSV | |
| | | MHEALHNRYTQKSLSLSPGK | |
| CD3hi | First Half | EVQLVQSGAEVKKPGESLKISCKASGYSFTNYWMNW | 82 |
| BSP2-2 | Antibody | VRQMPGKGLEWMGMIHPSDSEIRLNQKFQGQVTLSV | |
| arm | Heavy Chain | DKSIGTAYMQWSSLKASDTAMYYCSRWYYLSSPMD | |
| | (includes Fc | YWGQGTTVTVSSASTKGPSVFPLAPSSKSTSGGTAA | |
| | sequence) | LGCLVKDYFPEPVTVSWNSGALTSGVHTFPAVLQSS | |
| | | GLYSLSSVVTVPSSSLGTQTYICNVNHKPSNTKVDKR | |
| | | VEPKSCDKTHTCPPCPAPELLGGPSVFLFPPKPKDTL | |
| | | MISRTPEVTCVVVAVSHEDPEVKFNWYVDGVEVHNA | |
| | | KTKPREEQYASTYRVVSVLTVLHQDWLNGKEYKCKV | |
| | | SNKALAAPIEKTISKAKGQPREPQVCTLPPSREEMTK | |
| | | NQVSLSCAVKGFYPSDIAVEWESNGQPENNYKTTPP | |
| | | VLDSDGSFFLVSKLTVDKSRWQQGNVFSCSVMHEAL | |
| | | HNHYTQKSLSLSPGK | |
| | First Half | EIVMTQSPATLSVSPGERATLSCRASQDVGTAVAWY | 69 |
| | Antibody | QQKPGQAPRLLIYWASTRHTGIPARFSGSGSGTEFTL | |
| | Light Chain | TISSLQSEDFAVYFCQQYSSYPYTFGQGTKLEIKRTVA | |
| | | APSVFIFPPSDEQLKSGTASVVCLLNNFYPREAKVQW | |
| | | KVDNALQSGNSQESVTEQDSKDSTYSLSSTLTLSKAD | |
| | | YEKHKVYACEVTHQGLSSPVTKSFNRGEC | |
| | Second Half | EVQLVESGGGLVQPGGSLKLSCAASGFTFNTYAMN | 81 |
| | Antibody | WVRQASGKGLEWVGRIRSKYNNYATYYADSVKDRFT | |
| | (includes Fc | ISRDDSKSTLYLQMNSLKTEDTAVYYCVRHGNFGNSY | |
| | sequence) | VSWFAYWGQGTLVTVSSGGGGSGGGGSGGGGSG | |
| | | GGGSQAVVTQEPSLTVSPGGTVTLTCRSSTGAVTTS | |
| | | NYANWVQQKPGQAPRGLIGGTNKRAPWTPARFSGS | |
| | | LLGDKAALTLSGAQPEDEAEYFCALWYSNLWVFGGG | |
| | | TKLTVLGGGGSDKTHTCPPCPAPELLGGPSVFLFPPK | |
| | | PKDTLMISRTPEVTCVVVAVSHEDPEVKFNWYVDGV | |
| | | EVHNAKTKPREEQYASTYRVVSVLTVLHQDWLNGKE | |
| | | YKCKVSNKALAAPIEKTISKAKGQPREPQVYTLPPCR | |
| | | EEMTKNQVSLWCLVKGFYPSDIAVEWESNGQPENNY | |
| | | KTTPPVLDSDGSFFLYSKLTVDKSRWQQGNVFSCSV | |
| | | MHEALHNRYTQKSLSLSPGK | |

### 8.1.1.1.2. Expression and Purification

BBMs and TBMs were expressed transiently by co-transfection of the respective chains in HEK293 cells. Briefly, transfection of the cells with the heavy and light chain plasmids was performed using PEI as transfection reagent with a final DNA:PEI ratio of 1:3. 1 mg of plasmid per liter of culture was used for transfection of cultures having 2.0 million cells/mL of serum media. After 5 days of expression, BBMs and TBMs were harvested by clarification of the media via centrifugation and filtration. Purification was performed via anti-OH 1 affinity batch binding (CaptureSelect IgG-CH1 Affinity Matrix, Thermo-Fisher Scientific, Waltham, MA, USA) or Protein A (rProteinA Sepharose, Fast flow, GE Healthcare, Uppsala, Sweden) batch binding using 1 ml resin/100 mL supernatant. The protein was allowed to bind for a minimum of 2 hours with gentle mixing, and the supernatant loaded onto a gravity filtration column. The resin was washed with 20-50 CV of PBS. BBMs and TBMs were eluted with 20 CV of 50 mM citrate, 90 mM NaCl pH 3.2. 50 mM sucrose. The eluted BBM and TBM fractions were adjusted to pH 5.5 with 1 M sodium citrate 50 mM sucrose. Preparative size exclusion chromatography was performed using Hi Load 16/60 Superdex 200 grade column (GE Healthcare Life Sciences, Uppsala, Sweden) as a final polishing step when aggregates were present. To confirm that the identity of the proteins of the BBMs and TBMs expressed matched the predicted masses for the primary amino acid sequences, proteins were analyzed by high-performance liquid chromatography coupled to mass spectrometry.

### 8.1.1.1.3. CD3 Affinity Measurements

The affinity of the CD3hi, CD3med, and CD3lo mAbs to CD3 were determined at 25° C. using a Biacore T200 system. Briefly, anti-hFc IgG1 was immobilized on a CM5 chip. After capturing CD3-Fc (1 μg/ml in HBS-EP+ buffer, flow rate of 50 μl/min, with a 30 second injection time) kinetic data was acquired by subsequent injections of 1:2 dilution series of the different antibodies in HBS-EP+ buffer.

Data were evaluated using the Biacore T200 evaluation software version 1.0. The raw data were double referenced, i.e. the response of the measuring flow cell was corrected for the response of the reference flow cell, and in a second step the response of a blank injection was subtracted. Finally, the sensorgrams were fitted by applying 1:1 binding model to calculate kinetic rate constants and dissociation equilibrium constants. Rₘₐₓ was set at local. Data were processed individually for each run.

### 8.1.2. Example 1B: Additional BBM and TBM Constructs

A one-arm BBM having a CD3 ABM and a CD19 ABM (CD3hi BSP1, shown schematically in FIG. 3C) and a TBM corresponding to CD3hi TSP1 but with a lysozyme binding arm in place of the CD19 binding arm (CD3hi TSP1C) were produced. The amino acid sequences of the CD3hi BSP1 and CD3hi TSP1C constructs are shown in Table 20B.

TABLE 20B
Amino acid sequences

| | Chain | | SEQ |
|---|---|---|---|
| Construct | Descrip- | | ID |
| Name | tion | Amino Acid Sequence | NO: |
| CD3hi | First | QVQLVQSGAEVKKPGASVKVSCKAS | 74 |
| BSP1 | Half | GYTFTTYWIQWVRQAPGQRLEWMGA | |
| | Anti- | VYPGDADTRYTQKFQGRVTLTADRS | |
| | body | ASTAYMELSSLRSEDTAVYYCGRDA | |
| | Heavy | GLEYYALDYWGQGTLVTVSSASTKG | |
| | Chain | PSVFPLAPSSKSTSGGTAALGCLVK | |
| | (in- | DYFPEPVTVSWNSGALTSGVHTFPA | |
| | cludes | VLQSSGLYSLSSVVTVPSSSLGTQT | |
| | Fc se- | YICNVNHKPSNTKVDKRVEPKSCGG | |
| | quence) | GGSGGGGSEVQLVESGGGLVQPGGS | |
| | | LKLSCAASGFTFNTYAMNWVRQASG | |
| | | KGLEWVGRIRSKYNNYATYYADSVK | |
| | | DRFTISRDDSKSTLYLQMNSLKTED | |
| | | TAVYYCVRHGNFGNSYVSWFAYWGQ | |
| | | GTLVTVSSGGGGSGGGGSGGGGSGG | |
| | | GGSQAVVTQEPSLTVSPGGTVTLTC | |
| | | RSSTGAVTTSNYANWVQQKPGQAPR | |
| | | GLIGGTNKRAPWTPARFSGSLLGDK | |
| | | AALTLSGAQPEDEAEYFCALWYSNL | |
| | | WVFGGGTKLTVLGGGGSDKTHTCPP | |
| | | CPAPELLGGPSVFLFPPKPKDTLMI | |
| | | SRTPEVTCVVVAVSHEDPEVKFNWY | |
| | | VDGVEVHNAKTKPREEQYASTYRVV | |
| | | SVLTVLHQDWLNGKEYKCKVSNKAL | |
| | | AAPIEKTISKAKGQPREPQVCTLPP | |
| | | SREEMTKNQVSLSCAVKGFYPSDIA | |
| | | VEWESNGQPENNYKTTPPVLDSDGS | |
| | | FFLVSKLTVDKSRWQQGNVFSCSVM | |
| | | HEALHNHYTQKSLSLSPGK | |
| | First | EIVMTQSPATLSVSPGERATLSCRA | 64 |
| | Half | SQDVGTAVAWYQQKPGQAPRLLIYW | |
| | Anti- | ASTRHTGIPARFSGSGSGTEFTLTI | |
| | body | SSLQSEDFAVYFCQQYANFPLYTFG | |
| | Light | QGTKLEIKRTVAAPSVFIFPPSDEQ | |
| | Chain | LKSGTASVVCLLNNFYPREAKVQWK | |
| | | VDNALQSGNSQESVTEQDSKDSTYS | |
| | | LSSTLTLSKADYEKHKVYACEVTHQ | |
| | | GLSSPVTKSFNRGEC | |
| | Second | DKTHTCPPCPAPELLGGPSVFLFPP | 83 |
| | Half | KPKDTLMISRTPEVTCVVVAVSHED | |
| | Anti- | PEVKFNWYVDGVEVHNAKTKPREEQ | |
| | body | YASTYRVVSVLTVLHQDWLNGKEYK | |
| | (in- | CKVSNKALAAPIEKTISKAKGQPRE | |
| | cludes | PQVYTLPPCREEMTKNQVSLWCLVK | |
| | Fc se- | GFYPSDIAVEWESNGQPENNYKTTP | |
| | quence) | PVLDSDGSFFLYSKLTVDKSRWQQG | |
| | | NVFSCSVMHEALHNRYTQKSLSLSP | |
| | | GK | |
| CD3hi | First | QVQLQQSGPGLVKPSQTLSLTCAIS | 84 |
| TSP1C | Half | GDSVSSNSAAWSWIRQSPGRGLEWL | |
| | Anti- | GRIYYRSKWYNDYAVSVKSRITINP | |
| | body | DTSKNQFSLQLNSVTPEDTAVYYCA | |
| | Heavy | RLDHRYHEDTVYPGMDVWGQGTLVT | |
| | Chain | VSSASTKGPSVFPLAPSSKSTSGGT | |
| | (in- | AALGCLVKDYFPEPVTVSWNSGALT | |
| | cludes | SGVHTFPAVLQSSGLYSLSSVVTVP | |
| | Fc se- | SSSLGTQTYICNVNHKPSNTKVDKR | |
| | quence) | VEPKSCGGGGSGGGGSEVQLVESGG | |
| | | GLVQPGGSLKLSCAASGFTFNTYAM | |
| | | NWVRQASGKGLEWVGRIRSKYNNYA | |
| | | TYYADSVKDRFTISRDDSKSTLYLQ | |
| | | MNSLKTEDTAVYYCVRHGNFGNSYV | |
| | | SWFAYWGQGTLVTVSSGGGGSGGGG | |
| | | SGGGGSGGGGSQAVVTQEPSLTVSP | |
| | | GGTVTLTCRSSTGAVTTSNYANWVQ | |
| | | QKPGQAPRGLIGGTNKRAPWTPARF | |
| | | SGSLLGDKAALTLSGAQPEDEAEYF | |
| | | CALWYSNLWVFGGGTKLTVLGGGGS | |
| | | DKTHTCPPCPAPELLGGPSVFLFPP | |
| | | KPKDTLMISRTPEVTCVVVAVSHED | |
| | | PEVKFNWYVDGVEVHNAKTKPREEQ | |
| | | YASTYRVVSVLTVLHQDWLNGKEYK | |
| | | CKVSNKALAAPIEKTISKAKGQPRE | |
| | | PQVCTLPPSREEMTKNQVSLSCAVK | |
| | | GFYPSDIAVEWESNGQPENNYKTTP | |
| | | PVLDSDGSFFLVSKLTVDKSRWQQG | |
| | | NVFSCSVMHEALHNHYTQKSLSLSP | |
| | | GK | |
| | First | DIELTQPPSVSVAPGQTARISCSGD | 85 |
| | Half | NLPAYTVTWYQQKPGQAPVLVIYDD | |
| | Anti- | SDRPSGIPERFSGSNSGNTATLTIS | |
| | body | GTQAEDEADYYCASWDPSSGVVFGG | |
| | Light | GTKLTVLGQPKAAPSVTLFPPSSEE | |
| | Chain | LQANKATLVCLISDFYPGAVTVAWK | |
| | | ADSSPVKAGVETTTPSKQSNNKYAA | |
| | | SSYLSLTPEQWKSHRSYSCQVTHEG | |
| | | STVEKTVAPTECS | |
| | Second | SQQIYGVVYGNVTFHVPSNVPLKEV | 75 |
| | Half | LWKKQKDKVAELENSEFRAFSSFKN | |
| | Anti- | RVYLDTVSGSLTIYNLTSSDEDEYE | |
| | body | MESPNITDTMKFFLYVLESGGGGSD | |
| | (in- | KTHTCPPCPAPELLGGPSVFLFPPK | |
| | cludes | PKDTLMISRTPEVTCVVVAVSHEDP | |
| | Fc se- | EVKFNWYVDGVEVHNAKTKPREEQY | |
| | quence) | ASTYRVVSVLTVLHQDWLNGKEYKC | |
| | | KVSNKALAAPIEKTISKAKGQPREP | |
| | | QVYTLPPCREEMTKNQVSLWCLVKG | |
| | | FYPSDIAVEWESNGQPENNYKTTPP | |
| | | VLDSDGSFFLYSKLTVDKSRWQQGN | |
| | | VFSCSVMHEALHNRYTQKSLSLSPG | |
| | | K | |

Additionally, CD3hi TSP1, CD3med T P1, D3hi B P1, and D3hi T P1 constructs were each produced in a second version having a second half antibody sequence varying from the second half antibody sequence for the construct set forth in Table 20A-2 (in the case of CD3med TSP1 and CD3hi TSP1) or Table 20B (in the case of CD3hi BSP1 and CD3hi TSP1C) by one amino acid in the Fc sequence. Specifically, the second half antibody sequences in Table 20A-2 and Table 20B have an arginine residue where the second versions have a histidine residue. The arginine residue was included in the constructs to facilitate purification via Protein A binding. The versions of the constructs set forth in Table 20A-2 and Table 20B are referred to herein as “R variants” and the versions of the constructs set forth in Table 200, below, are referred to herein as “H variants.” It is believed that the functional activity of a construct's R variant does not differ significantly from the functional activity of its H variant. Nucleotide sequences encoding H variants of CD3hi TSP1, CD3med TSP1, CD3hi SP, and CD3hi TSP1C are shown in Table 20D.

TABLE 20C
Amino acid sequences (H variants)

| | | | SEQ |
|---|---|---|---|
| Construct | Chain | | ID |
| Name | Description | Amino Acid Sequence | NO: |
| CD3hi | First Half | QVQLVQSGAEVKKPGASVKVSCKASGYTFTTYWIQWVRQ | 74 |
| TSP1 | Antibody | APGQRLEWMGAVYPGDADTRYTQKFQGRVTLTADRSAST | |
| (H variant) | Heavy Chain | AYMELSSLRSEDTAVYYCGRDAGLEYYALDYWGQGTLVT | |
| | (includes Fc | VSSASTKGPSVFPLAPSSKSTSGGTAALGCLVKDYFPEPVT | |
| | sequence) | VSWNSGALTSGVHTFPAVLQSSGLYSLSSVVTVPSSSLGT | |
| | | QTYICNVNHKPSNTKVDKRVEPKSCGGGGSGGGGSEVQL | |
| | | VESGGGLVQPGGSLKLSCAASGFTFNTYAMNWRQASGK | |
| | | GLEWVGRIRSKYNNYATYYADSVKDRFTISRDDSKSTLYLQ | |
| | | MNSLKTEDTAVYYCVRHGNFGNSYVSWFAYWGQGTLVTV | |
| | | SSGGGGSGGGGSGGGGSGGGGSQAVVTQEPSLTVSPG | |
| | | GTVTLTCRSSTGAVTTSNYANWVQQKPGQAPRGLIGGTN | |
| | | KRAPWTPARFSGSLLGDKAALTLSGAQPEDEAEYFCALWY | |
| | | SNLWVFGGGTKLTVLGGGGSDKTHTCPPCPAPELLGGPS | |
| | | VFLFPPKPKDTLMISRTPEVTCVVVAVSHEDPEVKFNWYVD | |
| | | GVEVHNAKTKPREEQYASTYRVVSVLTVLHQDWLNGKEY | |
| | | KCKVSNKALAAPIEKTISKAKGQPREPQVCTLPPSREEMTK | |
| | | NQVSLSCAVKGFYPSDIAVEWESNGQPENNYKTTPPVLDS | |
| | | DGSFFLVSKLTVDKSRWQQGNVFSCSVMHEALHNHYTQK | |
| | | SLSLSPGK | |
| | First Half | EIVMTQSPATLSVSPGERATLSCRASQDVGTAVAWYQQKP | 64 |
| | Antibody | GQAPRLLIYWASTRHTGIPARFSGSGSGTEFTLTISSLQSE | |
| | Light Chain | DFAVYFCQQYANFPLYTFGQGTKLEIKRTVAAPSVFIFPPS | |
| | | DEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | Second Half | SQQIYGVVYGNVTFHVPSNVPLKEVLWKKQKDKVAELENS | 86 |
| | Antibody | EFRAFSSFKNRVYLDTVSGSLTIYNLTSSDEDEYEMESPNIT | |
| | (includes Fc | DTMKFFLYVLESGGGGSDKTHTCPPCPAPELLGGPSVFLF | |
| | sequence) | PPKPKDTLMISRTPEVTCVVVAVSHEDPEVKFNWYVDGVE | |
| | | VHNAKTKPREEQYASTYRVVSVLTVLHQDWLNGKEYKCKV | |
| | | SNKALAAPIEKTISKAKGQPREPQVYTLPPCREEMTKNQVS | |
| | | LWCLVKGFYPSDIAVEWESNGQPENNYKTTPPVLDSDGSF | |
| | | FLYSKLTVDKSRWQQGNVFSCSVMHEALHNHYTQKSLSLS | |
| | | PGK | |
| CD3med | First Half | QVQLVQSGAEVKKPGASVKVSCKASGYTFTTYWIQWVRQ | 76 |
| TSP1 | Antibody | APGQRLEWMGAVYPGDADTRYTQKFQGRVTLTADRSAST | |
| (H variant) | Heavy Chain | AYMELSSLRSEDTAVYYCGRDAGLEYYALDYWGQGTLVT | |
| | (includes Fc | VSSASTKGPSVFPLAPSSKSTSGGTAALGCLVKDYFPEPVT | |
| | sequence) | VSWNSGALTSGVHTFPAVLQSSGLYSLSSVVTVPSSSLGT | |
| | | QTYICNVNHKPSNTKVDKRVEPKSCGGGGSGGGGSEVQL | |
| | | VESGGGLVQPGGSLKLSCAASGFTFNTYAMNWVRQASGK | |
| | | GLEWVGRIRSKYNNYATYYADSVKDRFTISRDDSKNTAYL | |
| | | QMNSLKTEDTAVYYCVRHGNFGNSYVSWFAHWGQGTLV | |
| | | TVSSGGGGSGGGGSGGGGSGGGGSQAVVTQEPSLTVSP | |
| | | GGTVTLTCGSSTGAVTSSNYANWVQQKPGQAPRGLIGGT | |
| | | NKRAPWTPARFSGSLLGGKAALTLSGAQPEDEAEYYCAL | |
| | | WYSNLWVFGGGTKLTVLGGGGSDKTHTCPPCPAPELLGG | |
| | | PSVFLFPPKPKDTLMISRTPEVTCVVVAVSHEDPEVKFNWY | |
| | | VDGVEVHNAKTKPREEQYASTYRVVSVLTVLHQDWLNGK | |
| | | EYKCKVSNKALAAPIEKTISKAKGQPREPQVCTLPPSREEM | |
| | | TKNQVSLSCAVKGFYPSDIAVEWESNGQPENNYKTTPPVL | |
| | | DSDGSFFLVSKLTVDKSRWQQGNVFSCSVMHEALHNHYT | |
| | | QKSLSLSPGK | |
| | First Half | EIVMTQSPATLSVSPGERATLSCRASQDVGTAVAWYQQKP | 64 |
| | Antibody | GQAPRLLIYWASTRHTGIPARFSGSGSGTEFTLTISSLQSE | |
| | Light Chain | DFAVYFCQQYANFPLYTFGQGTKLEIKRTVAAPSVFIFPPS | |
| | | DEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | Second Half | SQQIYGVVYGNVTFHVPSNVPLKEVLWKKQKDKVAELENS | 86 |
| | Antibody | EFRAFSSFKNRVYLDTVSGSLTIYNLTSSDEDEYEMESPNIT | |
| | (includes Fc | DTMKFFLYVLESGGGGSDKTHTCPPCPAPELLGGPSVFLF | |
| | sequence) | PPKPKDTLMISRTPEVTCVVVAVSHEDPEVKFNWYVDGVE | |
| | | VHNAKTKPREEQYASTYRVVSVLTVLHQDWLNGKEYKCKV | |
| | | SNKALAAPIEKTISKAKGQPREPQVYTLPPCREEMTKNQVS | |
| | | LWCLVKGFYPSDIAVEWESNGQPENNYKTTPPVLDSDGSF | |
| | | FLYSKLTVDKSRWQQGNVFSCSVMHEALHNHYTQKSLSLS | |
| | | PGK | |
| CD3hi | First Half | QVQLVQSGAEVKKPGASVKVSCKASGYTFTTYWIQWVRQ | 74 |
| BSP1 | Antibody | APGQRLEWMGAVYPGDADTRYTQKFQGRVTLTADRSAST | |
| (H variant) | Heavy Chain | AYMELSSLRSEDTAVYYCGRDAGLEYYALDYWGQGTLVT | |
| | (includes Fc | VSSASTKGPSVFPLAPSSKSTSGGTAALGCLVKDYFPEPVT | |
| | sequence) | VSWNSGALTSGVHTFPAVLQSSGLYSLSSVVTVPSSSLGT | |
| | | QTYICNVNHKPSNTKVDKRVEPKSCGGGGSGGGGSEVQL | |
| | | VESGGGLVQPGGSLKLSCAASGFTFNTYAMNWVRQASGK | |
| | | GLEWVGRIRSKYNNYATYYADSVKDRFTISRDDSKSTLYLQ | |
| | | MNSLKTEDTAVYYCVRHGNFGNSYVSWFAYWGQGTLVTV | |
| | | SSGGGGSGGGGSGGGGSGGGGSQAVVTQEPSLTVSPG | |
| | | GTVTLTCRSSTGAVTTSNYANWVQQKPGQAPRGLIGGTN | |
| | | KRAPWTPARFSGSLLGDKAALTLSGAQPEDEAEYFCALWY | |
| | | SNLWVFGGGTKLTVLGGGGSDKTHTCPPCPAPELLGGPS | |
| | | VFLFPPKPKDTLMISRTPEVTCVVVAVSHEDPEVKFNWYVD | |
| | | GVEVHNAKTKPREEQYASTYRVVSVLTVLHQDWLNGKEY | |
| | | KCKVSNKALAAPIEKTISKAKGQPREPQVCTLPPSREEMTK | |
| | | NQVSLSCAVKGFYPSDIAVEWESNGQPENNYKTTPPVLDS | |
| | | DGSFFLVSKLTVDKSRWQQGNVFSCSVMHEALHNHYTQK | |
| | | SLSLSPGK | |
| | First Half | EIVMTQSPATLSVSPGERATLSCRASQDVGTAVAWYQQKP | 64 |
| | Antibody | GQAPRLLIYWASTRHTGIPARFSGSGSGTEFTLTISSLQSE | |
| | Light Chain | DFAVYFCQQYANFPLYTFGQGTKLEIKRTVAAPSVFIFPPS | |
| | | DEQLKSGTASVVCLLNNFYPREAKVQWKVDNALQSGNSQ | |
| | | ESVTEQDSKDSTYSLSSTLTLSKADYEKHKVYACEVTHQG | |
| | | LSSPVTKSFNRGEC | |
| | Second Half | DKTHTCPPCPAPELLGGPSVFLFPPKPKDTLMISRTPEVTC | 87 |
| | Antibody | VVVAVSHEDPEVKFNWYVDGVEVHNAKTKPREEQYASTY | |
| | (includes Fc | RVVSVLTVLHQDWLNGKEYKCKVSNKALAAPIEKTISKAKG | |
| | sequence) | QPREPQVYTLPPCREEMTKNQVSLWCLVKGFYPSDIAVE | |
| | | WESNGQPENNYKTTPPVLDSDGSFFLYSKLTVDKSRWQQ | |
| | | GNVFSCSVMHEALHNHYTQKSLSLSPGK | |
| CD3hi | First Half | QVQLQQSGPGLVKPSQTLSLTCAISGDSVSSNSAAWSWIR | 84 |
| TSP1C | Antibody | QSPGRGLEWLGRIYYRSKWYNDYAVSVKSRITINPDTSKN | |
| (H variant) | Heavy Chain | QFSLQLNSVTPEDTAVYYCARLDHRYHEDTVYPGMDVWG | |
| | (includes Fc | QGTLVTVSSASTKGPSVFPLAPSSKSTSGGTAALGCLVKD | |
| | sequence) | YFPEPVTVSWNSGALTSGVHTFPAVLQSSGLYSLSSVVTV | |
| | | PSSSLGTQTYICNVNHKPSNTKVDKRVEPKSCGGGGSGG | |
| | | GGSEVQLVESGGGLVQPGGSLKLSCAASGFTFNTYAMNW | |
| | | VRQASGKGLEWVGRIRSKYNNYATYYADSVKDRFTISRDD | |
| | | SKSTLYLQMNSLKTEDTAVYYCVRHGNFGNSYVSWFAYW | |
| | | GQGTLVTVSSGGGGSGGGGSGGGGSGGGGSQAVVTQE | |
| | | PSLTVSPGGTVTLTCRSSTGAVTTSNYANWVQQKPGQAP | |
| | | RGLIGGTNKRAPWTPARFSGSLLGDKAALTLSGAQPEDEA | |
| | | EYFCALWYSNLWVFGGGTKLTVLGGGGSDKTHTCPPCPA | |
| | | PELLGGPSVFLFPPKPKDTLMISRTPEVTCVVVAVSHEDPE | |
| | | VKFNWYVDGVEVHNAKTKPREEQYASTYRVVSVLTVLHQ | |
| | | DWLNGKEYKCKVSNKALAAPIEKTISKAKGQPREPQVCTLP | |
| | | PSREEMTKNQVSLSCAVKGFYPSDIAVEWESNGQPENNY | |
| | | KTTPPVLDSDGSFFLVSKLTVDKSRWQQGNVFSCSVMHE | |
| | | ALHNHYTQKSLSLSPGK | |
| | First Half | DIELTQPPSVSVAPGQTARISCSGDNLPAYTVTWYQQKPG | 85 |
| | Antibody | QAPVLVIYDDSDRPSGIPERFSGSNSGNTATLTISGTQAED | |
| | Light Chain | EADYYCASWDPSSGVVFGGGTKLTVLGQPKAAPSVTLFPP | |
| | | SSEELQANKATLVCLISDFYPGAVTVAWKADSSPVKAGVET | |
| | | TTPSKQSNNKYAASSYLSLTPEQWKSHRSYSCQVTHEGST | |
| | | VEKTVAPTECS | |
| | Second Half | SQQIYGVVYGNVTFHVPSNVPLKEVLWKKQKDKVAELENS | 86 |
| | Antibody | EFRAFSSFKNRVYLDTVSGSLTIYNLTSSDEDEYEMESPNIT | |
| | (includes Fc | DTMKFFLYVLESGGGGSDKTHTCPPCPAPELLGGPSVFLF | |
| | sequence) | PPKPKDTLMISRTPEVTCVVVAVSHEDPEVKFNWYVDGVE | |
| | | VHNAKTKPREEQYASTYRVVSVLTVLHQDWLNGKEYKCKV | |
| | | SNKALAAPIEKTISKAKGQPREPQVYTLPPCREEMTKNQVS | |
| | | LWCLVKGFYPSDIAVEWESNGQPENNYKTTPPVLDSDGSF | |
| | | FLYSKLTVDKSRWQQGNVFSCSVMHEALHNHYTQKSLSLS | |
| | | PGK | |

TABLE 20D
Nucleotide sequences (H variants)

| | | | SEQ |
|---|---|---|---|
| Construct | Chain | | ID |
| Name | Description | Nucleic Acid Sequence | NO: |
| CD3hi | First Half | ATGCCACTGCTGCTTCTACTGCCACTCCTGTGGGCAGGA | 88 |
| TSP1 | Antibody | GCACTGGCCCAAGTGCAACTGGTGCAGTCCGGTGCCGA | |
| (H variant) | Heavy Chain | AGTGAAGAAGCCCGGTGCCTCTGTGAAGGTGTCCTGCAA | |
| | (includes | GGCGTCGGGATACACGTTCACCACTTACTGGATTCAGTG | |
| | signal | GGTCAGACAGGCCCCGGGACAGAGACTGGAGTGGATGG | |
| | peptide | GAGCCGTGTACCCCGGAGATGCAGACACTCGCTACACCC | |
| | sequence) | AGAAGTTCCAGGGCCGCGTGACTTTGACCGCCGACAGAA | |
| | | GCGCCAGCACCGCCTACATGGAGCTTTCATCCCTCCGGA | |
| | | GCGAGGATACTGCCGTATACTATTGCGGAAGGGATGCCG | |
| | | GCCTGGAATACTATGCCCTCGACTACTGGGGACAGGGGA | |
| | | CCCTCGTGACTGTGTCCAGCGCGAGCACCAAGGGCCCC | |
| | | AGCGTGTTCCCGCTGGCCCCATCATCCAAGTCCACCTCG | |
| | | GGAGGGACTGCTGCGCTCGGTTGCCTTGTGAAGGACTAC | |
| | | TTCCCCGAGCCCGTGACTGTGTCGTGGAACAGCGGGGC | |
| | | TCTGACCAGCGGGGTTCACACCTTTCCCGCCGTGCTGCA | |
| | | GTCCTCGGGACTCTACAGCCTGTCCTCCGTGGTCACGGT | |
| | | CCCGTCGTCGTCGCTGGGGACCCAGACCTACATTTGCAA | |
| | | CGTGAACCACAAACCCTCCAACACAAAAGTGGACAAAAG | |
| | | GGTGGAACCTAAGTCCTGTGGAGGGGGTGGATCAGGCG | |
| | | GAGGAGGATCGGAAGTCCAGCTCGTCGAATCAGGGGGA | |
| | | GGGCTTGTGCAACCAGGAGGCTCCCTCAAGCTGTCTTGC | |
| | | GCAGCGTCCGGTTTCACTTTCAACACTTATGCGATGAATT | |
| | | GGGTCCGCCAAGCCAGTGGGAAGGGCCTGGAGTGGGTC | |
| | | GGACGGATCAGATCCAAGTACAACAACTACGCGACATAC | |
| | | TACGCCGACTCCGTGAAGGATCGCTTCACCATCAGCCGG | |
| | | GATGACTCCAAGAGCACCTTGTACCTCCAAATGAACAGC | |
| | | CTTAAGACCGAGGACACTGCGGTGTACTACTGCGTGAGA | |
| | | CACGGCAACTTCGGAAACTCCTACGTGTCCTGGTTCGCC | |
| | | TACTGGGGACAGGGCACCCTTGTCACTGTGTCAAGCGGA | |
| | | GGCGGTGGTTCGGGTGGCGGAGGTTCCGGAGGAGGAG | |
| | | GTTCGGGCGGTGGTGGATCACAGGCCGTCGTGACTCAG | |
| | | GAACCATCCCTGACTGTGTCCCCCGGTGGAACCGTGACC | |
| | | CTCACCTGTCGCTCCTCAACCGGAGCCGTGACCACCTCC | |
| | | AACTACGCTAATTGGGTGCAGCAGAAGCCAGGACAAGCC | |
| | | CCACGGGGACTGATTGGGGGCACCAACAAGAGGGCTCC | |
| | | TTGGACCCCAGCCCGCTTCTCGGGCTCCCTGTTGGGCGA | |
| | | CAAGGCCGCTCTGACCCTGTCCGGTGCACAGCCGGAGG | |
| | | ATGAAGCCGAATACTTCTGCGCGCTGTGGTACTCCAACC | |
| | | TCTGGGTGTTCGGCGGAGGGACCAAGCTGACTGTGTTG | |
| | | GGAGGAGGGGGGAGTGACAAGACTCACACGTGTCCGCC | |
| | | TTGCCCAGCACCCGAGCTACTGGGAGGACCGAGCGTGT | |
| | | TCCTGTTTCCCCCGAAGCCGAAGGATACCCTGATGATCT | |
| | | CCCGCACTCCTGAAGTGACTTGCGTGGTGGTGGCAGTGT | |
| | | CCCACGAGGACCCGGAAGTCAAGTTTAATTGGTACGTGG | |
| | | ATGGCGTGGAGGTGCACAACGCAAAGACCAAGCCTCGC | |
| | | GAGGAGCAGTACGCCAGCACCTACCGGGTGGTGTCCGT | |
| | | CCTGACGGTGCTGCACCAGGACTGGCTGAACGGGAAGG | |
| | | AGTACAAGTGCAAAGTGTCAAATAAGGCTTTGGCCGCCC | |
| | | CTATTGAGAAAACCATCTCAAAGGCCAAGGGCCAACCCA | |
| | | GGGAACCTCAAGTGTGCACCCTCCCACCTTCGCGAGAAG | |
| | | AGATGACCAAGAACCAGGTGTCCCTGTCCTGCGCCGTGA | |
| | | AGGGCTTCTACCCCTCCGATATCGCCGTGGAGTGGGAAT | |
| | | CTAACGGACAGCCGGAGAACAACTACAAGACCACTCCGC | |
| | | CGGTGCTGGACAGCGACGGCTCCTTCTTCCTCGTGTCGA | |
| | | AACTGACCGTGGACAAGTCACGGTGGCAGCAGGGCAAT | |
| | | GTGTTCAGCTGCTCAGTCATGCATGAGGCCCTCCACAAC | |
| | | CACTACACTCAGAAGTCCCTGTCGCTTTCCCCCGGAAAA | |
| | First Half | ATGTCGGTCCTGACCCAAGTGCTGGCCCTCCTTCTCCTG | 89 |
| | Antibody | TGGCTGACCGGGACCAGATGCGAAATCGTCATGACTCAG | |
| | Light Chain | AGCCCGGCAACCCTGTCCGTGAGCCCTGGAGAACGGGC | |
| | (includes | CACTCTGAGCTGTCGGGCGTCACAGGACGTGGGAACTG | |
| | signal | CCGTGGCCTGGTATCAGCAGAAGCCGGGACAGGCTCCT | |
| | peptide | AGGTTGCTCATCTACTGGGCGTCCACTCGCCACACCGGA | |
| | sequence) | ATCCCAGCCCGCTTCTCCGGCTCGGGTTCTGGCACCGAG | |
| | | TTCACCCTGACCATTTCCTCCCTCCAATCCGAGGATTTCG | |
| | | CCGTGTACTTCTGCCAACAATACGCCAACTTCCCCCTGTA | |
| | | CACATTTGGCCAGGGGACCAAGCTGGAGATTAAGCGTAC | |
| | | GGTGGCCGCTCCCAGCGTGTTCATCTTCCCCCCCAGCGA | |
| | | CGAGCAGCTGAAGAGCGGCACCGCCAGCGTGGTGTGCC | |
| | | TGCTGAACAACTTCTACCCCCGGGAGGCCAAGGTGCAGT | |
| | | GGAAGGTGGACAACGCCCTGCAGAGCGGCAACAGCCAG | |
| | | GAGAGCGTCACCGAGCAGGACAGCAAGGACTCCACCTA | |
| | | CAGCCTGAGCAGCACCCTGACCCTGAGCAAGGCCGACT | |
| | | ACGAGAAGCATAAGGTGTACGCCTGCGAGGTGACCCACC | |
| | | AGGGCCTGTCCAGCCCCGTGACCAAGAGCTTCAACAGG | |
| | | GGCGAGTGC | |
| | Second Half | ATGCCTCTGCTGCTCCTGCTGCCTCTGCTCTGGGCCGGA | 90 |
| | Antibody | GCTTTGGCATCACAGCAAATCTACGGCGTGGTGTACGGC | |
| | (includes | AACGTGACCTTCCATGTCCCCTCCAATGTGCCGCTGAAG | |
| | signal | GAAGTGCTCTGGAAGAAGCAGAAGGACAAGGTCGCGGA | |
| | peptide | ACTGGAAAACTCCGAGTTTCGCGCCTTCTCCTCCTTCAAA | |
| | sequence) | AACCGGGTGTACCTGGACACCGTGTCCGGGAGCCTTACT | |
| | | ATCTACAACCTGACCTCCTCGGACGAGGATGAGTATGAG | |
| | | ATGGAGAGCCCAAACATTACCGACACCATGAAGTTCTTCC | |
| | | TCTACGTGCTGGAATCGGGTGGAGGCGGAAGCGATAAG | |
| | | ACTCACACGTGTCCACCTTGTCCCGCACCCGAACTCCTG | |
| | | GGGGGACCTTCCGTGTTTCTCTTCCCCCCTAAACCGAAG | |
| | | GACACCTTGATGATCTCCCGCACTCCTGAAGTGACCTGT | |
| | | GTGGTGGTGGCCGTGTCCCACGAGGACCCAGAAGTCAA | |
| | | GTTTAATTGGTACGTGGACGGAGTCGAGGTGCACAACGC | |
| | | GAAAACCAAACCGCGGGAGGAGCAGTACGCCTCCACCTA | |
| | | CCGGGTGGTGTCCGTCCTCACTGTGCTGCACCAGGACTG | |
| | | GCTCAACGGAAAGGAGTACAAGTGCAAAGTGTCCAACAA | |
| | | AGCCTTGGCGGCCCCAATCGAAAAGACGATCTCCAAGGC | |
| | | CAAGGGACAGCCGCGCGAACCTCAAGTCTACACCCTGCC | |
| | | TCCTTGCCGCGAGGAAATGACCAAGAACCAGGTGTCACT | |
| | | GTGGTGTCTGGTCAAGGGATTCTACCCTTCCGATATCGC | |
| | | AGTGGAGTGGGAAAGCAACGGCCAACCAGAGAACAACTA | |
| | | TAAGACCACACCCCCGGTGCTCGATTCCGACGGCTCATT | |
| | | CTTCCTGTACTCCAAGCTGACCGTGGACAAGTCACGGTG | |
| | | GCAGCAGGGGAACGTGTTCAGCTGCTCCGTGATGCATGA | |
| | | AGCCCTGCACAATCATTACACTCAGAAGTCCCTGTCGCT | |
| | | GAGCCCCGGAAAA | |
| CD3med | First Half | ATGCCACTGCTGCTTCTACTGCCACTCCTGTGGGCAGGA | 91 |
| TSP1 | Antibody | GCACTGGCCCAAGTGCAACTGGTGCAGTCCGGTGCCGA | |
| (H variant) | Heavy Chain | AGTGAAGAAGCCCGGTGCCTCTGTGAAGGTGTCCTGCAA | |
| | (includes | GGCGTCGGGATACACGTTCACCACTTACTGGATTCAGTG | |
| | signal | GGTCAGACAGGCCCCGGGACAGAGACTGGAGTGGATGG | |
| | peptide | GAGCCGTGTACCCCGGAGATGCAGACACTCGCTACACCC | |
| | sequence) | AGAAGTTCCAGGGCCGCGTGACTTTGACCGCCGACAGAA | |
| | | GCGCCAGCACCGCCTACATGGAGCTTTCATCCCTCCGGA | |
| | | GCGAGGATACTGCCGTATACTATTGCGGAAGGGATGCCG | |
| | | GCCTGGAATACTATGCCCTCGACTACTGGGGACAGGGGA | |
| | | CCCTCGTGACTGTGTCCAGCGCGAGCACCAAGGGCCCG | |
| | | AGCGTGTTCCCATTGGCCCCGTCGTCAAAGTCCACCTCT | |
| | | GGCGGAACTGCGGCTCTGGGATGTCTCGTGAAGGACTA | |
| | | CTTTCCGGAACCCGTGACTGTGTCCTGGAACAGCGGCGC | |
| | | CCTCACTTCCGGCGTGCATACCTTCCCTGCCGTGCTGCA | |
| | | GTCCTCCGGCCTGTACAGCCTCAGCAGCGTCGTGACTGT | |
| | | GCCCTCCTCGTCCTTGGGCACCCAGACCTACATCTGCAA | |
| | | CGTCAACCACAAGCCCTCGAACACCAAAGTGGATAAGCG | |
| | | GGTGGAACCCAAGAGCTGTGGAGGGGGTGGCTCAGGAG | |
| | | GAGGGGGATCCGAAGTGCAGCTCGTGGAGTCCGGAGGA | |
| | | GGCCTGGTGCAGCCTGGGGGATCCCTCAAGCTTAGCTG | |
| | | CGCCGCATCAGGCTTCACCTTCAACACCTACGCCATGAA | |
| | | CTGGGTCCGCCAAGCATCCGGAAAGGGCCTGGAATGGG | |
| | | TCGGGAGAATCAGATCCAAGTACAACAACTACGCCACGT | |
| | | ACTACGCGGACTCCGTCAAGGACCGGTTCACTATTAGCC | |
| | | GGGATGACTCCAAGAATACCGCGTACCTTCAGATGAACT | |
| | | CGCTCAAAACCGAGGACACTGCCGTGTATTACTGCGTGC | |
| | | GGCACGGAAACTTCGGGAACAGTTACGTGTCCTGGTTCG | |
| | | CCCATTGGGGTCAAGGCACCCTGGTCACCGTGTCCTCGG | |
| | | GTGGTGGTGGCTCCGGTGGAGGAGGATCGGGGGGTGG | |
| | | AGGATCTGGGGGAGGCGGATCACAGGCGGTCGTGACTC | |
| | | AGGAGCCCTCCCTGACCGTGTCGCCTGGTGGCACCGTG | |
| | | ACTCTGACTTGCGGAAGCTCAACAGGCGCCGTGACCTCC | |
| | | TCGAACTACGCCAACTGGGTGCAACAGAAGCCGGGACAA | |
| | | GCCCCTAGGGGACTGATCGGGGGGACCAACAAGCGCGC | |
| | | TCCGTGGACTCCCGCGAGGTTCTCCGGAAGCCTCCTGG | |
| | | GAGGGAAGGCAGCCCTGACCCTGTCCGGAGCTCAGCCA | |
| | | GAAGATGAGGCCGAGTACTATTGCGCCCTGTGGTACTCG | |
| | | AATCTGTGGGTGTTTGGAGGCGGCACCAAGCTGACCGTC | |
| | | CTGGGTGGTGGCGGAAGCGACAAGACTCACACGTGTCC | |
| | | GCCTTGCCCAGCACCCGAGCTACTGGGAGGACCGAGCG | |
| | | TGTTCCTGTTTCCCCCGAAGCCGAAGGATACCCTGATGA | |
| | | TCTCCCGCACTCCTGAAGTGACTTGCGTGGTGGTGGCAG | |
| | | TGTCCCACGAGGACCCGGAAGTCAAGTTTAATTGGTACG | |
| | | TGGATGGCGTGGAGGTGCACAACGCAAAGACCAAGCCT | |
| | | CGCGAGGAGCAGTACGCCAGCACCTACCGGGTGGTGTC | |
| | | CGTCCTGACGGTGCTGCACCAGGACTGGCTGAACGGGA | |
| | | AGGAGTACAAGTGCAAAGTGTCAAATAAGGCTTTGGCCG | |
| | | CCCCTATTGAGAAAACCATCTCAAAGGCCAAGGGCCAAC | |
| | | CCAGGGAACCTCAAGTGTGCACCCTCCCACCTTCGCGAG | |
| | | AAGAGATGACCAAGAACCAGGTGTCCCTGTCCTGCGCCG | |
| | | TGAAGGGCTTCTACCCCTCCGATATCGCCGTGGAGTGGG | |
| | | AATCTAACGGACAGCCGGAGAACAACTACAAGACCACTC | |
| | | CGCCGGTGCTGGACAGCGACGGCTCCTTCTTCCTCGTGT | |
| | | CGAAACTGACCGTGGACAAGTCACGGTGGCAGCAGGGC | |
| | | AATGTGTTCAGCTGCTCAGTCATGCATGAGGCCCTCCAC | |
| | | AACCACTACACTCAGAAGTCCCTGTCGCTTTCCCCCGGA | |
| | | AAA | |
| | First Half | GAAATCGTCATGACTCAGAGCCCGGCAACCCTGTCCGTG | 92 |
| | Antibody | AGCCCTGGAGAACGGGCCACTCTGAGCTGTCGGGCGTC | |
| | Light Chain | ACAGGACGTGGGAACTGCCGTGGCCTGGTATCAGCAGA | |
| | (includes | AGCCGGGACAGGCTCCTAGGTTGCTCATCTACTGGGCGT | |
| | signal | CCACTCGCCACACCGGAATCCCAGCCCGCTTCTCCGGCT | |
| | peptide | CGGGTTCTGGCACCGAGTTCACCCTGACCATTTCCTCCC | |
| | sequence) | TCCAATCCGAGGATTTCGCCGTGTACTTCTGCCAACAATA | |
| | | CGCCAACTTCCCCCTGTACACATTTGGCCAGGGGACCAA | |
| | | GCTGGAGATTAAGCGTACGGTGGCCGCTCCCAGCGTGTT | |
| | | CATCTTCCCCCCCAGCGACGAGCAGCTGAAGAGCGGCA | |
| | | CCGCCAGCGTGGTGTGCCTGCTGAACAACTTCTACCCCC | |
| | | GGGAGGCCAAGGTGCAGTGGAAGGTGGACAACGCCCTG | |
| | | CAGAGCGGCAACAGCCAGGAGAGCGTCACCGAGCAGGA | |
| | | CAGCAAGGACTCCACCTACAGCCTGAGCAGCACCCTGAC | |
| | | CCTGAGCAAGGCCGACTACGAGAAGCATAAGGTGTACGC | |
| | | CTGCGAGGTGACCCACCAGGGCCTGTCCAGCCCCGTGA | |
| | | CCAAGAGCTTCAACAGGGGCGAGTGC | |
| | Second Half | ATGCCTCTGCTGCTCCTGCTGCCTCTGCTCTGGGCCGGA | 90 |
| | Antibody | GCTTTGGCATCACAGCAAATCTACGGCGTGGTGTACGGC | |
| | (includes | AACGTGACCTTCCATGTCCCCTCCAATGTGCCGCTGAAG | |
| | signal | GAAGTGCTCTGGAAGAAGCAGAAGGACAAGGTCGCGGA | |
| | peptide | ACTGGAAAACTCCGAGTTTCGCGCCTTCTCCTCCTTCAAA | |
| | sequence) | AACCGGGTGTACCTGGACACCGTGTCCGGGAGCCTTACT | |
| | | ATCTACAACCTGACCTCCTCGGACGAGGATGAGTATGAG | |
| | | ATGGAGAGCCCAAACATTACCGACACCATGAAGTTCTTCC | |
| | | TCTACGTGCTGGAATCGGGTGGAGGCGGAAGCGATAAG | |
| | | ACTCACACGTGTCCACCTTGTCCCGCACCCGAACTCCTG | |
| | | GGGGGACCTTCCGTGTTTCTCTTCCCCCCTAAACCGAAG | |
| | | GACACCTTGATGATCTCCCGCACTCCTGAAGTGACCTGT | |
| | | GTGGTGGTGGCCGTGTCCCACGAGGACCCAGAAGTCAA | |
| | | GTTTAATTGGTACGTGGACGGAGTCGAGGTGCACAACGC | |
| | | GAAAACCAAACCGCGGGAGGAGCAGTACGCCTCCACCTA | |
| | | CCGGGTGGTGTCCGTCCTCACTGTGCTGCACCAGGACTG | |
| | | GCTCAACGGAAAGGAGTACAAGTGCAAAGTGTCCAACAA | |
| | | AGCCTTGGCGGCCCCAATCGAAAAGACGATCTCCAAGGC | |
| | | CAAGGGACAGCCGCGCGAACCTCAAGTCTACACCCTGCC | |
| | | TCCTTGCCGCGAGGAAATGACCAAGAACCAGGTGTCACT | |
| | | GTGGTGTCTGGTCAAGGGATTCTACCCTTCCGATATCGC | |
| | | AGTGGAGTGGGAAAGCAACGGCCAACCAGAGAACAACTA | |
| | | TAAGACCACACCCCCGGTGCTCGATTCCGACGGCTCATT | |
| | | CTTCCTGTACTCCAAGCTGACCGTGGACAAGTCACGGTG | |
| | | GCAGCAGGGGAACGTGTTCAGCTGCTCCGTGATGCATGA | |
| | | AGCCCTGCACAATCATTACACTCAGAAGTCCCTGTCGCT | |
| | | GAGCCCCGGAAAA | |
| CD3hi | First Half | ATGCCACTGCTGCTTCTACTGCCACTCCTGTGGGCAGGA | 88 |
| BSP1 | Antibody | GCACTGGCCCAAGTGCAACTGGTGCAGTCCGGTGCCGA | |
| (H variant) | Heavy Chain | AGTGAAGAAGCCCGGTGCCTCTGTGAAGGTGTCCTGCAA | |
| | (includes | GGCGTCGGGATACACGTTCACCACTTACTGGATTCAGTG | |
| | signal | GGTCAGACAGGCCCCGGGACAGAGACTGGAGTGGATGG | |
| | peptide | GAGCCGTGTACCCCGGAGATGCAGACACTCGCTACACCC | |
| | sequence) | AGAAGTTCCAGGGCCGCGTGACTTTGACCGCCGACAGAA | |
| | | GCGCCAGCACCGCCTACATGGAGCTTTCATCCCTCCGGA | |
| | | GCGAGGATACTGCCGTATACTATTGCGGAAGGGATGCCG | |
| | | GCCTGGAATACTATGCCCTCGACTACTGGGGACAGGGGA | |
| | | CCCTCGTGACTGTGTCCAGCGCGAGCACCAAGGGCCCC | |
| | | AGCGTGTTCCCGCTGGCCCCATCATCCAAGTCCACCTCG | |
| | | GGAGGGACTGCTGCGCTCGGTTGCCTTGTGAAGGACTAC | |
| | | TTCCCCGAGCCCGTGACTGTGTCGTGGAACAGCGGGGC | |
| | | TCTGACCAGCGGGGTTCACACCTTTCCCGCCGTGCTGCA | |
| | | GTCCTCGGGACTCTACAGCCTGTCCTCCGTGGTCACGGT | |
| | | CCCGTCGTCGTCGCTGGGGACCCAGACCTACATTTGCAA | |
| | | CGTGAACCACAAACCCTCCAACACAAAAGTGGACAAAAG | |
| | | GGTGGAACCTAAGTCCTGTGGAGGGGGTGGATCAGGCG | |
| | | GAGGAGGATCGGAAGTCCAGCTCGTCGAATCAGGGGGA | |
| | | GGGCTTGTGCAACCAGGAGGCTCCCTCAAGCTGTCTTGC | |
| | | GCAGCGTCCGGTTTCACTTTCAACACTTATGCGATGAATT | |
| | | GGGTCCGCCAAGCCAGTGGGAAGGGCCTGGAGTGGGTC | |
| | | GGACGGATCAGATCCAAGTACAACAACTACGCGACATAC | |
| | | TACGCCGACTCCGTGAAGGATCGCTTCACCATCAGCCGG | |
| | | GATGACTCCAAGAGCACCTTGTACCTCCAAATGAACAGC | |
| | | CTTAAGACCGAGGACACTGCGGTGTACTACTGCGTGAGA | |
| | | CACGGCAACTTCGGAAACTCCTACGTGTCCTGGTTCGCC | |
| | | TACTGGGGACAGGGCACCCTTGTCACTGTGTCAAGCGGA | |
| | | GGCGGTGGTTCGGGTGGCGGAGGTTCCGGAGGAGGAG | |
| | | GTTCGGGCGGTGGTGGATCACAGGCCGTCGTGACTCAG | |
| | | GAACCATCCCTGACTGTGTCCCCCGGTGGAACCGTGACC | |
| | | CTCACCTGTCGCTCCTCAACCGGAGCCGTGACCACCTCC | |
| | | AACTACGCTAATTGGGTGCAGCAGAAGCCAGGACAAGCC | |
| | | CCACGGGGACTGATTGGGGGCACCAACAAGAGGGCTCC | |
| | | TTGGACCCCAGCCCGCTTCTCGGGCTCCCTGTTGGGCGA | |
| | | CAAGGCCGCTCTGACCCTGTCCGGTGCACAGCCGGAGG | |
| | | ATGAAGCCGAATACTTCTGCGCGCTGTGGTACTCCAACC | |
| | | TCTGGGTGTTCGGCGGAGGGACCAAGCTGACTGTGTTG | |
| | | GGAGGAGGGGGGAGTGACAAGACTCACACGTGTCCGCC | |
| | | TTGCCCAGCACCCGAGCTACTGGGAGGACCGAGCGTGT | |
| | | TCCTGTTTCCCCCGAAGCCGAAGGATACCCTGATGATCT | |
| | | CCCGCACTCCTGAAGTGACTTGCGTGGTGGTGGCAGTGT | |
| | | CCCACGAGGACCCGGAAGTCAAGTTTAATTGGTACGTGG | |
| | | ATGGCGTGGAGGTGCACAACGCAAAGACCAAGCCTCGC | |
| | | GAGGAGCAGTACGCCAGCACCTACCGGGTGGTGTCCGT | |
| | | CCTGACGGTGCTGCACCAGGACTGGCTGAACGGGAAGG | |
| | | AGTACAAGTGCAAAGTGTCAAATAAGGCTTTGGCCGCCC | |
| | | CTATTGAGAAAACCATCTCAAAGGCCAAGGGCCAACCCA | |
| | | GGGAACCTCAAGTGTGCACCCTCCCACCTTCGCGAGAAG | |
| | | AGATGACCAAGAACCAGGTGTCCCTGTCCTGCGCCGTGA | |
| | | AGGGCTTCTACCCCTCCGATATCGCCGTGGAGTGGGAAT | |
| | | CTAACGGACAGCCGGAGAACAACTACAAGACCACTCCGC | |
| | | CGGTGCTGGACAGCGACGGCTCCTTCTTCCTCGTGTCGA | |
| | | AACTGACCGTGGACAAGTCACGGTGGCAGCAGGGCAAT | |
| | | GTGTTCAGCTGCTCAGTCATGCATGAGGCCCTCCACAAC | |
| | | CACTACACTCAGAAGTCCCTGTCGCTTTCCCCCGGAAAA | |
| | First Half | ATGTCGGTCCTGACCCAAGTGCTGGCCCTCCTTCTCCTG | 89 |
| | Antibody | TGGCTGACCGGGACCAGATGCGAAATCGTCATGACTCAG | |
| | Light Chain | AGCCCGGCAACCCTGTCCGTGAGCCCTGGAGAACGGGC | |
| | (includes | CACTCTGAGCTGTCGGGCGTCACAGGACGTGGGAACTG | |
| | signal | CCGTGGCCTGGTATCAGCAGAAGCCGGGACAGGCTCCT | |
| | peptide | AGGTTGCTCATCTACTGGGCGTCCACTCGCCACACCGGA | |
| | sequence) | ATCCCAGCCCGCTTCTCCGGCTCGGGTTCTGGCACCGAG | |
| | | TTCACCCTGACCATTTCCTCCCTCCAATCCGAGGATTTCG | |
| | | CCGTGTACTTCTGCCAACAATACGCCAACTTCCCCCTGTA | |
| | | CACATTTGGCCAGGGGACCAAGCTGGAGATTAAGCGTAC | |
| | | GGTGGCCGCTCCCAGCGTGTTCATCTTCCCCCCCAGCGA | |
| | | CGAGCAGCTGAAGAGCGGCACCGCCAGCGTGGTGTGCC | |
| | | TGCTGAACAACTTCTACCCCCGGGAGGCCAAGGTGCAGT | |
| | | GGAAGGTGGACAACGCCCTGCAGAGCGGCAACAGCCAG | |
| | | GAGAGCGTCACCGAGCAGGACAGCAAGGACTCCACCTA | |
| | | CAGCCTGAGCAGCACCCTGACCCTGAGCAAGGCCGACT | |
| | | ACGAGAAGCATAAGGTGTACGCCTGCGAGGTGACCCACC | |
| | | AGGGCCTGTCCAGCCCCGTGACCAAGAGCTTCAACAGG | |
| | | GGCGAGTGC | |
| | Second Half | ATGCCTCTGCTGCTCCTGCTGCCTCTGCTCTGGGCCGGA | 93 |
| | Antibody | GCTTTGGCAGATAAGACTCACACGTGTCCACCTTGTCCC | |
| | (includes | GCACCCGAACTCCTGGGGGGACCTTCCGTGTTTCTCTTC | |
| | signal | CCCCCTAAACCGAAGGACACCTTGATGATCTCCCGCACT | |
| | peptide | CCTGAAGTGACCTGTGTGGTGGTGGCCGTGTCCCACGA | |
| | sequence) | GGACCCAGAAGTCAAGTTTAATTGGTACGTGGACGGAGT | |
| | | CGAGGTGCACAACGCGAAAACCAAACCGCGGGAGGAGC | |
| | | AGTACGCCTCCACCTACCGGGTGGTGTCCGTCCTCACTG | |
| | | TGCTGCACCAGGACTGGCTCAACGGAAAGGAGTACAAGT | |
| | | GCAAAGTGTCCAACAAAGCCTTGGCGGCCCCAATCGAAA | |
| | | AGACGATCTCCAAGGCCAAGGGACAGCCGCGCGAACCT | |
| | | CAAGTCTACACCCTGCCTCCTTGCCGCGAGGAAATGACC | |
| | | AAGAACCAGGTGTCACTGTGGTGTCTGGTCAAGGGATTC | |
| | | TACCCTTCCGATATCGCAGTGGAGTGGGAAAGCAACGGC | |
| | | CAACCAGAGAACAACTATAAGACCACACCCCCGGTGCTC | |
| | | GATTCCGACGGCTCATTCTTCCTGTACTCCAAGCTGACC | |
| | | GTGGACAAGTCACGGTGGCAGCAGGGGAACGTGTTCAG | |
| | | CTGCTCCGTGATGCATGAAGCCCTGCACAATCATTACACT | |
| | | CAGAAGTCCCTGTCGCTGAGCCCCGGAAAA | |
| CD3hi | First Half | ATGCCTCTGCTGCTCCTGTTGCCCCTGCTGTGGGCTGGA | 94 |
| TSP1C | Antibody | GCCTTGGCCCAAGTGCAGCTTCAGCAGTCGGGACCCGG | |
| (H variant) | Heavy Chain | ACTCGTGAAGCCGTCGCAGACGCTGTCCCTGACCTGTGC | |
| | (includes | CATTAGCGGCGACTCCGTGAGCAGCAACAGCGCAGCCT | |
| | signal | GGTCCTGGATTCGGCAGTCACCTGGTCGGGGGCTTGAAT | |
| | peptide | GGCTGGGACGGATCTACTACCGCTCGAAATGGTATAACG | |
| | sequence) | ACTACGCCGTGTCTGTGAAGTCCAGGATCACCATCAACC | |
| | | CGGACACCTCCAAGAATCAGTTCTCCCTCCAACTGAACTC | |
| | | AGTGACCCCAGAGGACACCGCCGTCTACTACTGCGCGA | |
| | | GACTGGATCACCGCTACCATGAAGATACCGTGTACCCGG | |
| | | GGATGGACGTCTGGGGCCAGGGTACTCTCGTCACTGTGT | |
| | | CCTCCGCGTCCACTAAGGGCCCCAGCGTGTTCCCGCTG | |
| | | GCCCCATCATCCAAGTCCACCTCGGGAGGGACTGCTGC | |
| | | GCTCGGTTGCCTTGTGAAGGACTACTTCCCCGAGCCCGT | |
| | | GACTGTGTCGTGGAACAGCGGGGCTCTGACCAGCGGGG | |
| | | TTCACACCTTTCCCGCCGTGCTGCAGTCCTCGGGACTCT | |
| | | ACAGCCTGTCCTCCGTGGTCACGGTCCCGTCGTCGTCGC | |
| | | TGGGGACCCAGACCTACATTTGCAACGTGAACCACAAAC | |
| | | CCTCCAACACAAAAGTGGACAAAAGGGTGGAACCTAAGT | |
| | | CCTGTGGAGGGGGTGGATCAGGCGGAGGAGGATCGGAA | |
| | | GTCCAGCTCGTCGAATCAGGGGGAGGGCTTGTGCAACC | |
| | | AGGAGGCTCCCTCAAGCTGTCTTGCGCAGCGTCCGGTTT | |
| | | CACTTTCAACACTTATGCGATGAATTGGGTCCGCCAAGCC | |
| | | AGTGGGAAGGGCCTGGAGTGGGTCGGACGGATCAGATC | |
| | | CAAGTACAACAACTACGCGACATACTACGCCGACTCCGT | |
| | | GAAGGATCGCTTCACCATCAGCCGGGATGACTCCAAGAG | |
| | | CACCTTGTACCTCCAAATGAACAGCCTTAAGACCGAGGA | |
| | | CACTGCGGTGTACTACTGCGTGAGACACGGCAACTTCGG | |
| | | AAACTCCTACGTGTCCTGGTTCGCCTACTGGGGACAGGG | |
| | | CACCCTTGTCACTGTGTCAAGCGGAGGCGGTGGTTCGG | |
| | | GTGGCGGAGGTTCCGGAGGAGGAGGTTCGGGCGGTGGT | |
| | | GGATCACAGGCCGTCGTGACTCAGGAACCATCCCTGACT | |
| | | GTGTCCCCCGGTGGAACCGTGACCCTCACCTGTCGCTCC | |
| | | TCAACCGGAGCCGTGACCACCTCCAACTACGCTAATTGG | |
| | | GTGCAGCAGAAGCCAGGACAAGCCCCACGGGGACTGAT | |
| | | TGGGGGCACCAACAAGAGGGCTCCTTGGACCCCAGCCC | |
| | | GCTTCTCGGGCTCCCTGTTGGGCGACAAGGCCGCTCTGA | |
| | | CCCTGTCCGGTGCACAGCCGGAGGATGAAGCCGAATACT | |
| | | TCTGCGCGCTGTGGTACTCCAACCTCTGGGTGTTCGGCG | |
| | | GAGGGACCAAGCTGACTGTGTTGGGAGGAGGGGGGAGT | |
| | | GACAAGACTCACACGTGTCCGCCTTGCCCAGCACCCGAG | |
| | | CTACTGGGAGGACCGAGCGTGTTCCTGTTTCCCCCGAAG | |
| | | CCGAAGGATACCCTGATGATCTCCCGCACTCCTGAAGTG | |
| | | ACTTGCGTGGTGGTGGCAGTGTCCCACGAGGACCCGGA | |
| | | AGTCAAGTTTAATTGGTACGTGGATGGCGTGGAGGTGCA | |
| | | CAACGCAAAGACCAAGCCTCGCGAGGAGCAGTACGCCA | |
| | | GCACCTACCGGGTGGTGTCCGTCCTGACGGTGCTGCAC | |
| | | CAGGACTGGCTGAACGGGAAGGAGTACAAGTGCAAAGT | |
| | | GTCAAATAAGGCTTTGGCCGCCCCTATTGAGAAAACCATC | |
| | | TCAAAGGCCAAGGGCCAACCCAGGGAACCTCAAGTGTGC | |
| | | ACCCTCCCACCTTCGCGAGAAGAGATGACCAAGAACCAG | |
| | | GTGTCCCTGTCCTGCGCCGTGAAGGGCTTCTACCCCTCC | |
| | | GATATCGCCGTGGAGTGGGAATCTAACGGACAGCCGGA | |
| | | GAACAACTACAAGACCACTCCGCCGGTGCTGGACAGCGA | |
| | | CGGCTCCTTCTTCCTCGTGTCGAAACTGACCGTGGACAA | |
| | | GTCACGGTGGCAGCAGGGCAATGTGTTCAGCTGCTCAGT | |
| | | CATGCATGAGGCCCTCCACAACCACTACACTCAGAAGTC | |
| | | CCTGTCGCTTTCCCCCGGAAAA | |
| | First Half | ATGTCCGTGCTGACCCAAGTCTTGGCGCTGCTGCTGCTG | 95 |
| | Antibody | TGGCTCACTGGCACCCGCTGTGACATTGAACTGACCCAG | |
| | Light Chain | CCGCCTTCAGTGTCCGTGGCACCCGGACAGACCGCGAG | |
| | (includes | GATTAGCTGCTCCGGGGACAACCTCCCGGCCTACACTGT | |
| | signal | GACCTGGTATCAGCAGAAGCCCGGACAAGCCCCTGTGCT | |
| | peptide | TGTCATCTACGACGACTCGGATCGGCCAAGCGGCATCCC | |
| | sequence) | CGAGAGATTCTCCGGCTCGAACAGCGGGAACACCGCCA | |
| | | CGCTCACTATCTCGGGAACCCAGGCCGAAGATGAGGCTG | |
| | | ACTACTACTGCGCCTCATGGGATCCGTCCTCCGGAGTGG | |
| | | TGTTCGGTGGCGGAACTAAGCTGACCGTGCTGGGTCAGC | |
| | | CTAAGGCGGCGCCCTCAGTGACCCTGTTCCCTCCGTCGT | |
| | | CTGAAGAACTCCAGGCCAACAAGGCCACCCTCGTGTGCC | |
| | | TGATTTCGGACTTCTACCCGGGAGCCGTCACTGTGGCCT | |
| | | GGAAGGCCGACAGCAGCCCAGTGAAGGCCGGCGTGGAA | |
| | | ACTACCACCCCGTCCAAGCAGTCCAACAATAAGTACGCA | |
| | | GCCAGCTCCTACCTGTCCCTGACCCCCGAACAATGGAAG | |
| | | TCACACAGATCCTACTCCTGTCAAGTCACCCACGAGGGC | |
| | | AGCACTGTCGAAAAGACCGTGGCACCGACTGAGTGCTCG | |
| | Second Half | ATGCCTCTGCTGCTCCTGCTGCCTCTGCTCTGGGCCGGA | 90 |
| | Antibody | GCTTTGGCATCACAGCAAATCTACGGCGTGGTGTACGGC | |
| | (includes | AACGTGACCTTCCATGTCCCCTCCAATGTGCCGCTGAAG | |
| | signal | GAAGTGCTCTGGAAGAAGCAGAAGGACAAGGTCGCGGA | |
| | peptide | ACTGGAAAACTCCGAGTTTCGCGCCTTCTCCTCCTTCAAA | |
| | sequence) | AACCGGGTGTACCTGGACACCGTGTCCGGGAGCCTTACT | |
| | | ATCTACAACCTGACCTCCTCGGACGAGGATGAGTATGAG | |
| | | ATGGAGAGCCCAAACATTACCGACACCATGAAGTTCTTCC | |
| | | TCTACGTGCTGGAATCGGGTGGAGGCGGAAGCGATAAG | |
| | | ACTCACACGTGTCCACCTTGTCCCGCACCCGAACTCCTG | |
| | | GGGGGACCTTCCGTGTTTCTCTTCCCCCCTAAACCGAAG | |
| | | GACACCTTGATGATCTCCCGCACTCCTGAAGTGACCTGT | |
| | | GTGGTGGTGGCCGTGTCCCACGAGGACCCAGAAGTCAA | |
| | | GTTTAATTGGTACGTGGACGGAGTCGAGGTGCACAACGC | |
| | | GAAAACCAAACCGCGGGAGGAGCAGTACGCCTCCACCTA | |
| | | CCGGGTGGTGTCCGTCCTCACTGTGCTGCACCAGGACTG | |
| | | GCTCAACGGAAAGGAGTACAAGTGCAAAGTGTCCAACAA | |
| | | AGCCTTGGCGGCCCCAATCGAAAAGACGATCTCCAAGGC | |
| | | CAAGGGACAGCCGCGCGAACCTCAAGTCTACACCCTGCC | |
| | | TCCTTGCCGCGAGGAAATGACCAAGAACCAGGTGTCACT | |
| | | GTGGTGTCTGGTCAAGGGATTCTACCCTTCCGATATCGC | |
| | | AGTGGAGTGGGAAAGCAACGGCCAACCAGAGAACAACTA | |
| | | TAAGACCACACCCCCGGTGCTCGATTCCGACGGCTCATT | |
| | | CTTCCTGTACTCCAAGCTGACCGTGGACAAGTCACGGTG | |
| | | GCAGCAGGGGAACGTGTTCAGCTGCTCCGTGATGCATGA | |
| | | AGCCCTGCACAATCATTACACTCAGAAGTCCCTGTCGCT | |
| | | GAGCCCCGGAAAA | |

### 8.2. Example 2: Ability of BBMs to Elicit Redirected T-Cell Cytotoxic Activity (RTCC) Against CD19+ Target Cells

### 8.2.1. Materials and Methods

A RTCC assay with the BBMs of Example 1A was performed to measure the ability of the BBMs to elicit RTCC against CD19+ Nalm6-luc and Karpas422-luc cells. Nalm-6 is a human B cell precursor leukemia cell line and Karpas422 is a human B-cell non-hodgkin lymphoma cell line. Briefly, Nalm6 and Karpas422 cells engineered to express the firefly luciferase reporter gene were cultured in RPMI1640 culture media with 10% fetal bovine serum (FBS). 10,000 target cells with serial diluted BBMs or gH isotype antibody control (αgH-CD3hi) were seeded on 384-well flat-bottom microtiter plate. Primary human T cells were isolated from cryopreserved peripheral blood mononuclear cells (PBMCs) and expanded using anti-CD3 and anti-CD28 dynabeads (Thermo fisher, catalog #11131D) and subsequently cryopreserved. Expanded T cells were thawed and aliquoted to the plate to achieve an effector cell (i.e., T cell) to target cell (i.e., cancer cell) ratio (E:T ratio) of 3:1. Plates were incubated in a 37° C. incubator with 5% C02 overnight. Following the co-incubation, Bright Glo (Promega, catalog #E2620) was added to all wells and the luminescence signal was subsequently measured on an Envision (Perkin Elmer). Target cells with Bright Glo served as maximal signal. The percent RTCC of target cells was calculated using the following formula: [100−(sample/maximal signal)*100%].

### 8.2.2. Results

Results are shown in FIGS. 4A-4B. BBMs based on both NEG258 and NEG218 mediated RTCC activity against Nalm6-luc and Karpas422-luc cells whereas gH isotype antibody (control) was not active, as expected.

### 8.3. Example 3: Ability of BBMs to Elicit T-Cell Proliferation

### 8.3.1. Materials and Methods

The BBMs described in Example 1A, containing the variable regions of NEG258 and NEG218, were evaluated for their ability to induce T cell proliferation upon co-culture with CD19 expressing target cells. Briefly, Karpas422 and Nalm-6 target cells stably expressing firefly luciferase were irradiated on the day of the assay and plated at a density of 60,000 cells per well in a Costar 96 well plate (Corning, Cat #3904) in T Cell Media (TCM) [RPMI-1640 (ThermoFisher Scientific, Cat #11875-085), 10% FBS (Seradigm, Cat #1500-500), 1% L-Glutamine (Thermo Fisher Scientific, Cat #25830-081), 1% Non Essential Amino Acids (Thermo Fisher Scientific, Cat #11140-050), 1% Pen/Strep (Thermo Fisher Scientific, Cat #15070063), 1% HEPES (Thermo Fisher Scientific, Cat #15630080), Sodium Pyruvate (Thermo Fisher Scientific, Cat #11360-070), 0.1% Beta-mercaptoethanol (Thermo Fisher Scientific, Cat #21985-023)]. Peripheral blood mononuclear cells (PBMCs) previously isolated from Leukopak donors (Hemacare) and cryopreserved were thawed and Pan T cells were isolated by negative selection using the Pan T cell Isolation Kit, human [Miltenyi Biotec, Cat #130-096-535] following the manufacturer's protocol. Isolated T cells were labelled with 5 μM Cell Trace Violet (CTV) (Thermo Fisher Scientific, Cat #C34557) following the manufacturer's protocol and 60,000 CTV labeled T cells were co-cultured with 60,000 target cells to achieve an E:T ratio of 1:1. A dilution series of the NEG258- and NEG218-based BBMs and control binding molecules (αgH-CD3hi) ranging from 16 μM-10,000 μM was added to cells and the plates were incubated in a 5% C02, 37° C. incubator for 96 hrs. After incubation, the cells were harvested, treated with Human TruStain FcX (Fc Block) [Biolegend, Cat #422302] following manufacturer instructions and then stained with Fixable Viability Dye eFlour 780 (ThermoFisher Scientific, Cat #65-0865-14) by incubation at 4C for 30 mins. The cells were then washed twice using FACS Buffer and stained with PerCP-Cy5.5 conjugated anti-human CD3 mAb (Biolegend, Cat #317336) by incubation at 4° C. for 30 mins. The samples were then run on BD LSR Fortessa and analyzed using FlowJo to determine % proliferated CD3+ T cells based on CD3 staining and dilution of Cell Trace Violet dye.

### 8.3.2. Results

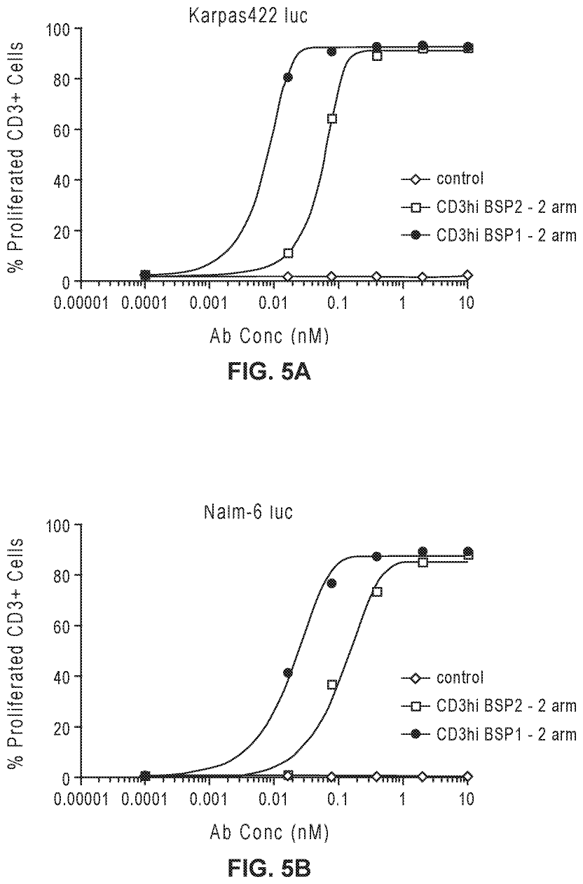

Both NEG258- and NEG218-based BBMs induced proliferation of T cells upon co-culture with two different CD19 expressing target cell lines (FIGS. 5A-5B). The T cell proliferation effect was dose-dependent, and the NEG258-based BBM showed more potent activity than the NEG218-based BBM. The control antibody did not induce any T cell proliferation indicating that CD19 target-specific engagement was required for the proliferation of T cells.

### 8.4. Example 4: Ability of TBMs to Elicit CD2 Dependent T Cell Activation

### 8.4.1. Materials & Methods

A Jurkat cell line (JNL, an immortalized human T-cell line) that stably expresses a luciferase reporter gene driven by the NFAT promoter was used to measure T cell activation. The level of CD2 expression in JNL cells was confirmed by flow cytometry (FIG. 6A). In order to generate CD2 knockout (KO) cells by CRISPR (Clustered Regularly Interspaced Short Palindromic Repeats), JNL cells were electroporated with a CD2 Cas9 ribonucleoprotein complex. CD2⁻ cells were subsequently sorted to enrich for a uniform CD2⁻ population (FIG. 6B). A JNL reporter assay with CD2⁺ and CD2⁻ JNL cells was then performed to measure bispecfic or trispecific construct-dependent T cell activation. In brief, 10,000 Nalm6 or Karpas422 cells with serial diluted BBMs or TBMs of Example 1A (i.e., R variants) were seeded on 384-well flat-bottom microtiter plate. JNL cells were then added to the plate to achieve effector to target ratio of 3:1. Plates were incubated at a 37° C. incubator with 5% CO₂ for overnight. Following the co-incubation, Bright Glo (Promega, catalog #E2620) was added to all wells and the luminescence signal was subsequently measured on an Envision (Perkin Elmer).

### 8.4.2. Results

Both BBMs and TBMs induced dose-dependent increase in luminescence when incubated with CD2 WT JNL cells, and the response level was higher with TBMs (FIGS. 6C-6F). When CD2-KO JNL cells were used as effector, decreased T cell activation was observed with TBMs as compared to corresponding BBMs, suggesting that the advantage of TBMs is dependent on CD2 expression on the T cells.

### 8.5. Example 5: Binding of NEG258- and NEG218-Based TBMs to Cyno B Cells

### 8.5.1. Materials and Methods

Cynomolgus (cyno) PBMCs (iQ Biosciences #IQB-MnPB102) were depleted of CD3+ cells using MACS positive selection (Miltenyi #130-092-012). The remaining cell population was resuspended in a FACS buffer. 100,000 cells per well were plated in a V-bottom 96-well plate, and incubated on ice for one hour with TBMs of Example 1A (i.e., R variants) at 1ug/mL. Following two washes with FACS buffer, the cells were incubated with Alexa-647 labeled anti-human Fc secondary antibody (Jackson Immuno #109-605-098) and cyno cross reactive FITC mouse anti-human CD20 antibody (BD Pharmingen #556632) for one hour on ice. Following two washes with FACS buffer, cells were resuspended in 100 μL of buffer and data was collected on a Beckman Coulter Cytoflex. Cells were analyzed using CytExper v2.3 and gated through CD20 positive population.

### 8.5.2. Results

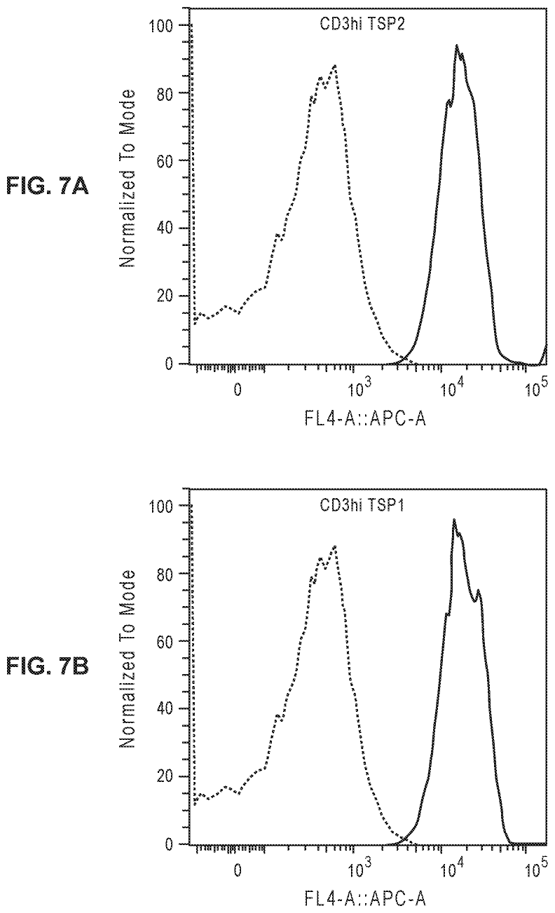

Due to their proximal evolutionary relationship to humans, cynomolgus monkeys are the most appropriate preclinical model to analyze the therapeutic effect and potential toxicity of antibody therapeutics, and therefore it is useful for antibodies in clinical development to bind to cynomolgus homolog of their human target. As shown in FIGS. 7A-7B, both the NEG258- and NEG218 TBMs bind to cyno B cells, indicating that the CD19 binding arm recognizes cyno CD19.

### 8.6. Example 6: Ability of TBMs to Induce T Cell Activation Upon Cyno B Cells Depletion in PBMCs

### 8.6.1. Materials & Methods

An ex vivo cyno B cell depletion assay was conducted to measure the ability of NEG258-based TBMs of Example 1 to lyse CD20 positive B cells in PBMCs (peripheral blood mononuclear cells). In brief, PBMCs were isolated from cynomolgus (cyno) monkey whole blood (BiolVT) using ficoll gradient centrifugation. Isolated PBMCs and serial diluted TBMs of Example 1A (i.e., R variants) were seeded on 96-well flat-bottom microtiter plate. Plates were incubated in a 37° C. incubator with 5% C02 overnight. After 24h of incubation, samples were harvested and simultaneously stained for CD3 and CD20 to identify B and T cells within the PBMC population. To allow quantitative analysis of the cell population, 75,600 counting beads were added prior to the acquisition by flow cytometry. For each sample, 20,000 beads were acquired in order to determine the absolute numbers of B cells. The percent B cell depletion was determined by calculation of the ratio between the number of B cells and the number of beads. For detection of T cell activation, the cells were stained with anti-CD3, anti-CD69 and anti-CD25 (Biolegend and BD Biosciences).

### 8.6.2. Results

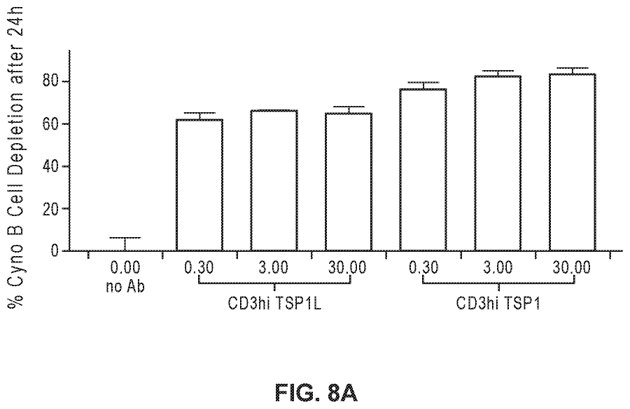

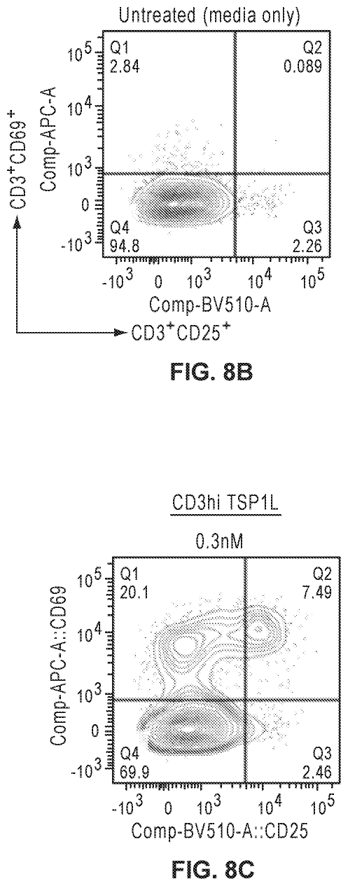

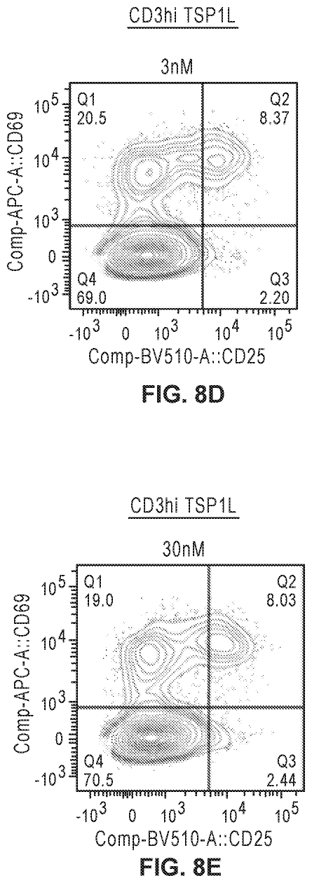

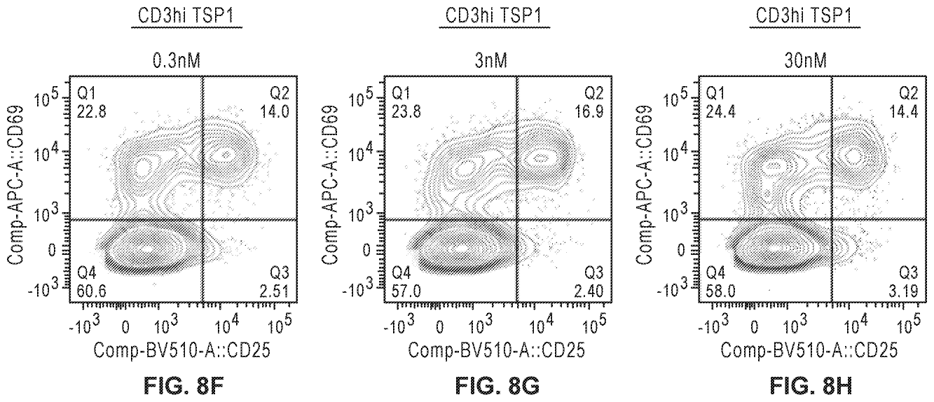

Both NEG258-based TBMs depleted cyno B cells (FIG. 8A) and induced activation of CD3+ T cells as evidence by upregulation of CD69 and CD25 expression (FIGS. 8C-8H). As expected, neither B cell depletion nor T cell activation occurred in the absence of added TBM. These results show both the ability of the NEG258-based TBMs induce activation of cyno T cells as well as the specificity of the activation.

### 8.7. Example 7: Re-Directed T Cell Cytotoxicity by CD19 TBMs

NEG258- and NEG218 based TBMs of Example 1A (i.e., R variants) (having CD3 ABMs with the VH and VL domains of an anti-CD3 antibody having an affinity to CD3 of 16 nM as measured by Biacore) were analyzed for their potential to induce T cell-mediated apoptosis in tumor target cells.

### 8.7.1. Materials and Methods

In one study, the TBMs were compared across multiple donor effector cells. Briefly, huCD19-expressing Nalm6 or Karpas422 target cells were engineered to overexpress firefly luciferase. Cells were harvested and resuspended in RPMI medium (Invitrogen #11875-093) with 10% FBS. 2,500 target cells per well were plated in a flat-bottom 384-well plate. Human pan T effector cells were isolated via MACS negative selection (Miltenyi Biotec #130-096-535) from two donors from cryopreserved PBMC (Cellular Technologies Limited #CTL-UP1) then added to the plate to obtain a final E:T ratio of 3:1 or 5:1. Co-cultured cells were incubated with a serial dilution of all constructs and controls. For normalization, average maximum luminescence refers to target cells co-incubated with effector cells, but without any test construct. After an incubation of 24, 48, 72 or 96 hr at 37° C., 5% C02, OneGlo luciferase substrate (Promega #E6120) was added to the plate. Luminescence was measured on an Envision plate reader after a 10 minute incubation. Percent specific lysis was calculated using the following equation: Specific lysis (%)=(1−(sample luminescence/average maximum luminescence))*100

### 8.7.2. Results

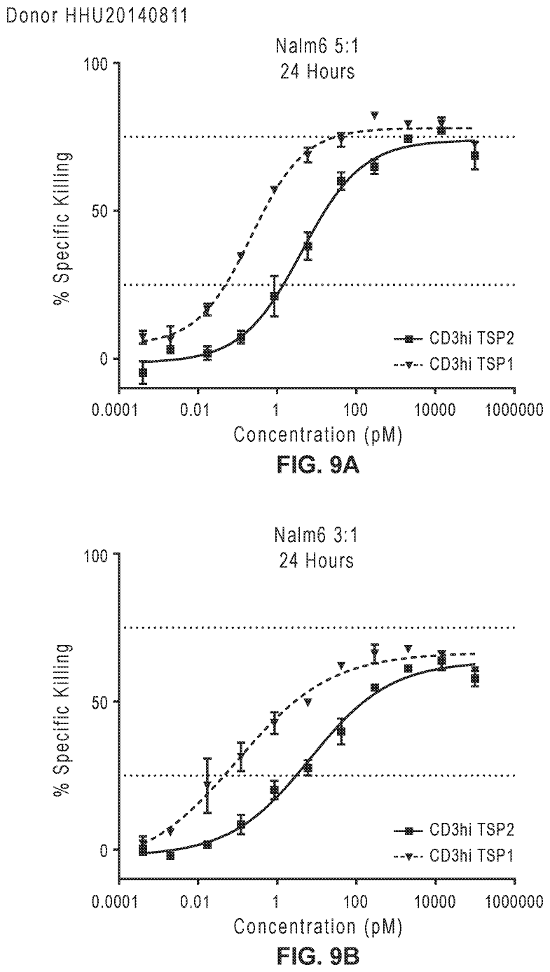

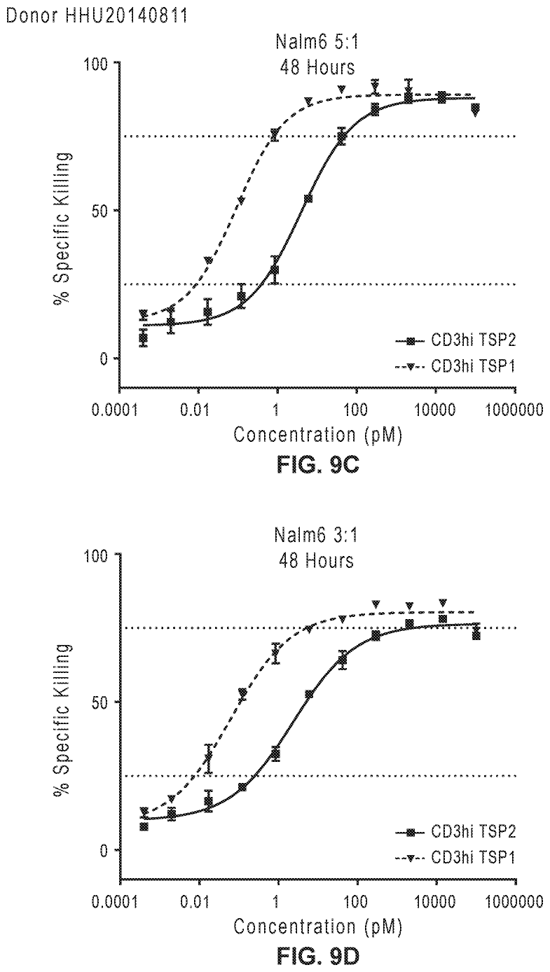

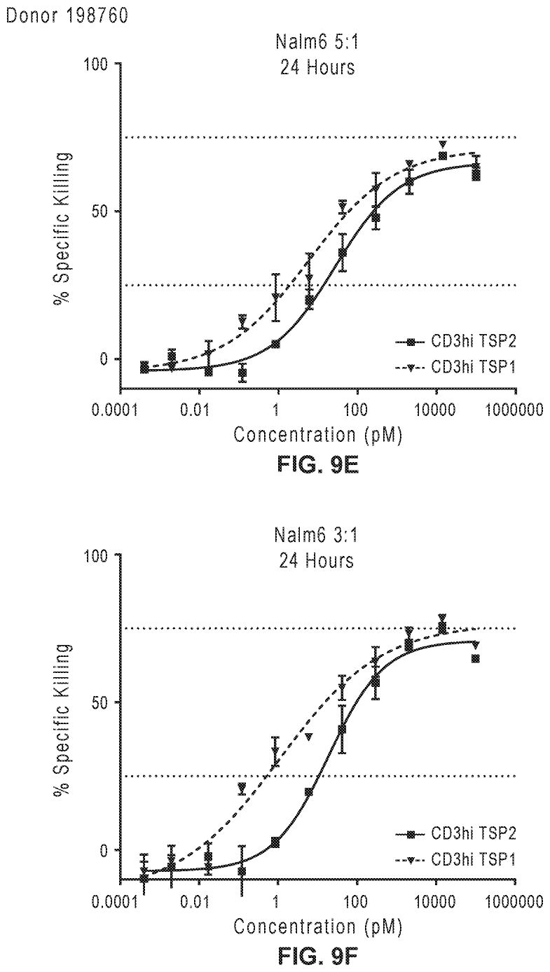

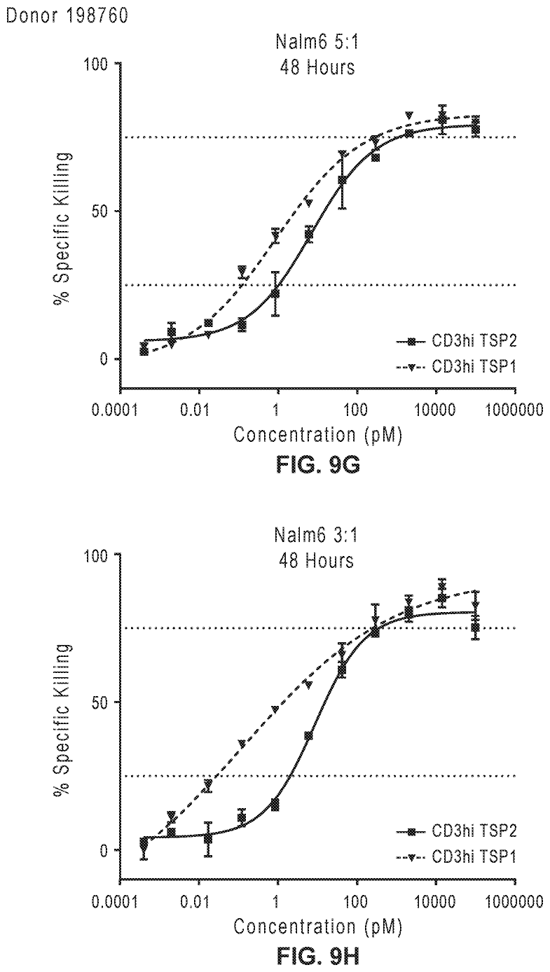

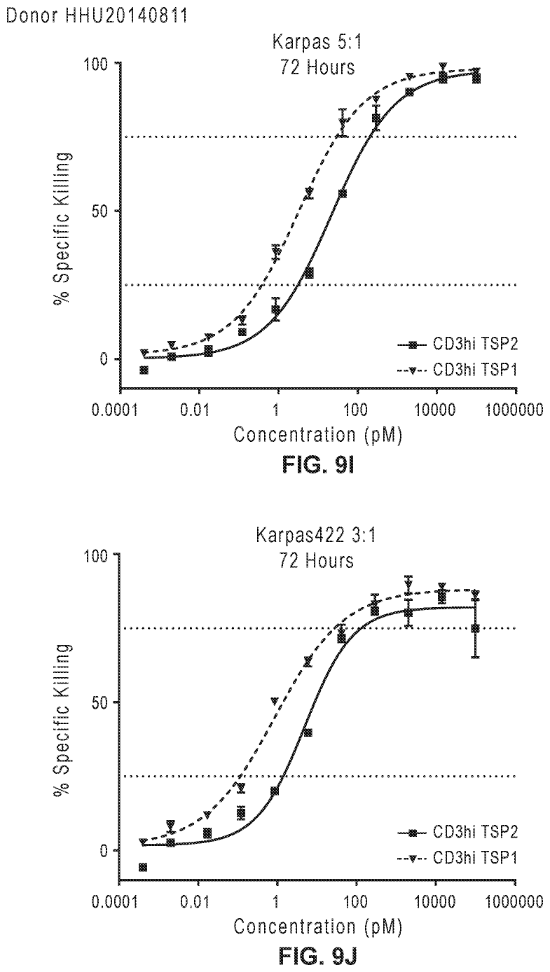

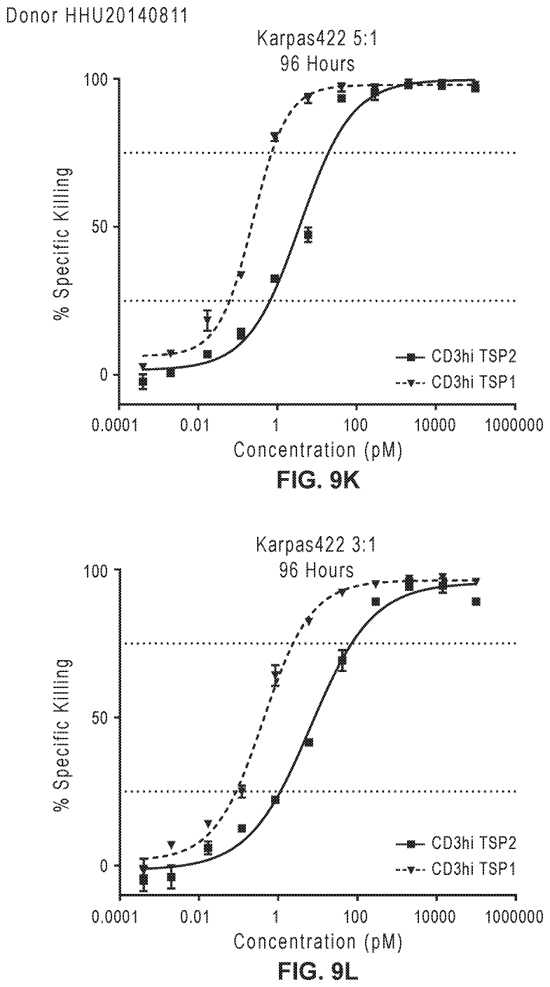

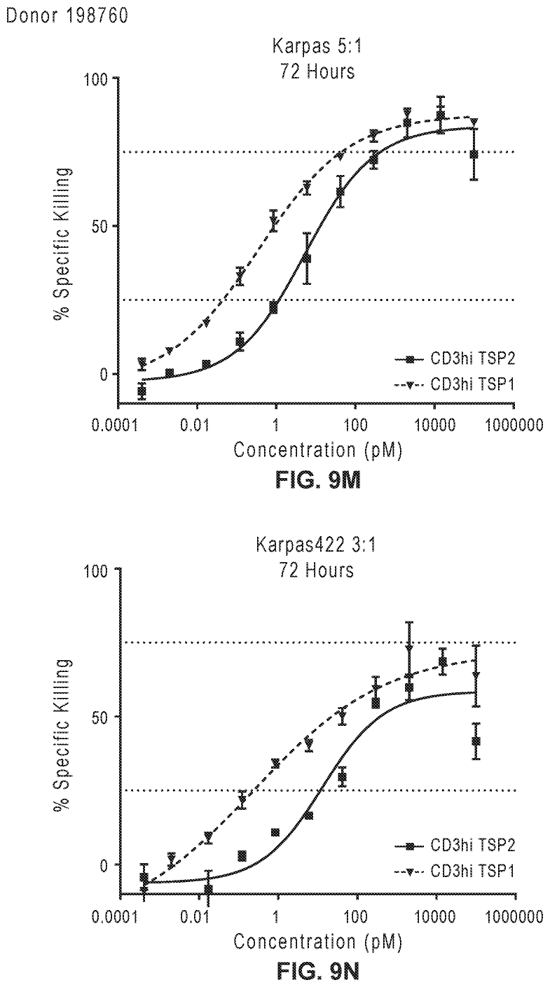

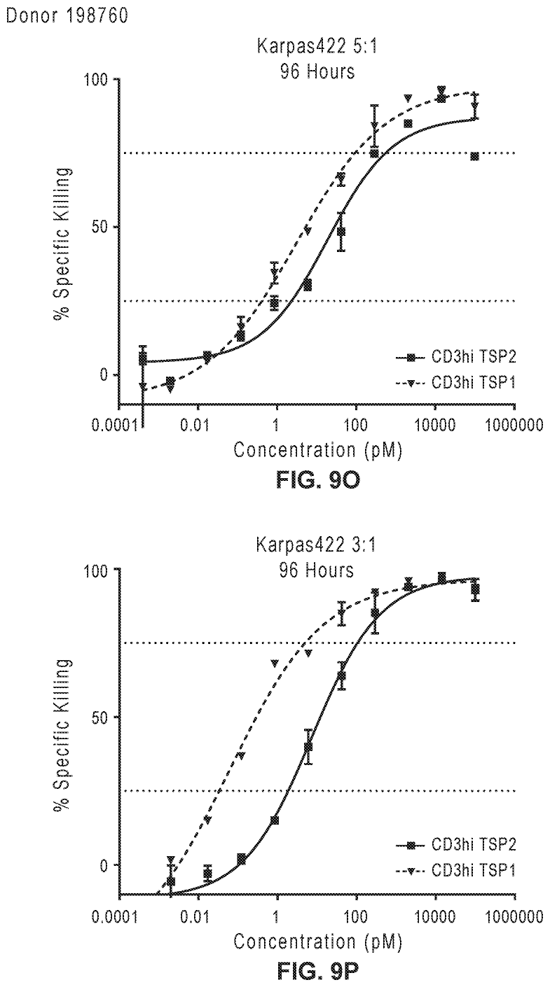

As shown in FIGS. 9A-9P, the TBMs show cytotoxic activity against both Nalm6 target cells (FIGS. 9A-9H) and Karpas422 cells (FIGS. 9I-P) at multiple time points, E:T ratios and effector T cell donors. The NEG258-based TBM appears to be more potent than the NEG218-based TBM.

### 8.8. Example 8: Re-Directed T Cell Cytotoxicity by TBMs with Different CD3 Affinities

The NEG258-based TBMs of Example 1A (i.e., R variants) with CD3 ABMs (comprising the VH and VL domains of anti-CD3 antibodies having affinities to CD3 of 16 nM, 30 nM and 48 nM as measured by Biacore) were analyzed for their potential to induce T cell-mediated apoptosis in tumor target cells.

### 8.8.1. Materials and Methods

In one study, the TBMs were compared across multiple donor effector cells. Briefly, huCD19-expressing Nalm6 and Karpas422 target cells were engineered to overexpress firefly luciferase. Cells were harvested and resuspended in RPMI medium (Invitrogen #11875-093) with 10% FBS. 2,500 target cells per well were plated in a flat-bottom 384-well plate. Human pan T effector cells were isolated via MACS negative selection (Miltenyi Biotec #130-096-535) from two donors from cryopreserved PBMCs (Cellular Technologies Limited #CTL-UP1), then added to the plate to obtain a final E:T ratio of 3:1 or 5:1. Co-cultured cells were incubated with serial dilutions of a TBM or control. For normalization, average maximum luminescence refers to target cells co-incubated with effector cells, but without any test construct. After an incubation of 24, 48, 72 or 96 hr at 37° C., 5% C02, OneGlo luciferase substrate (Promega #E6120) was added to the plate. Luminescence was measured on an Envision plate reader after a 10 minute incubation. Percent specific lysis was calculated using the following equation: Specific lysis (%)=(1−(sample luminescence/average maximum luminescence))*100

### 8.8.2. Results

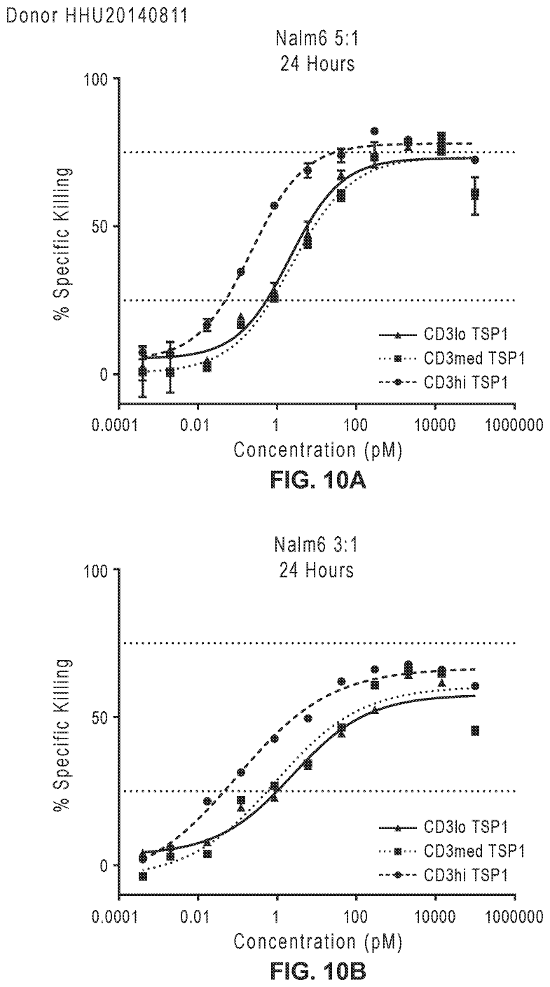

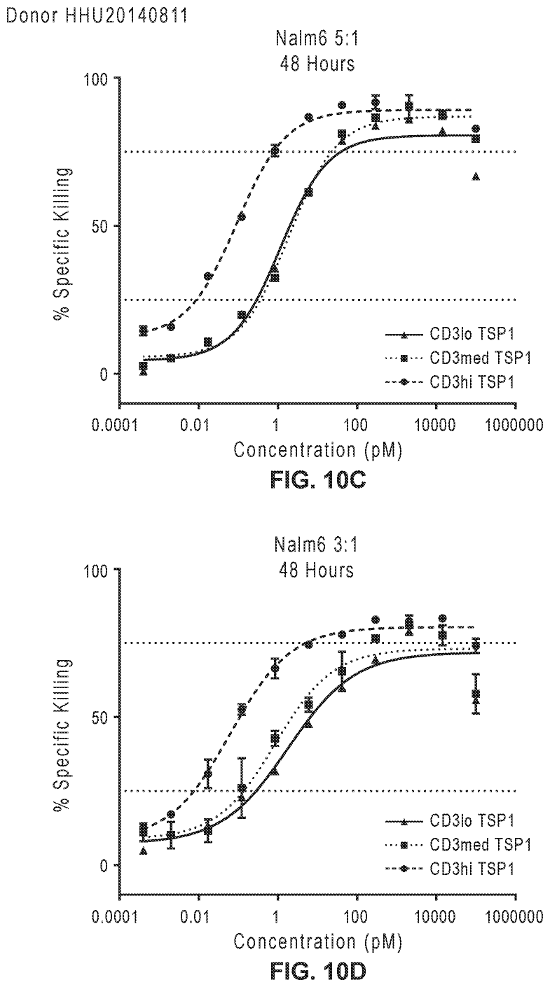

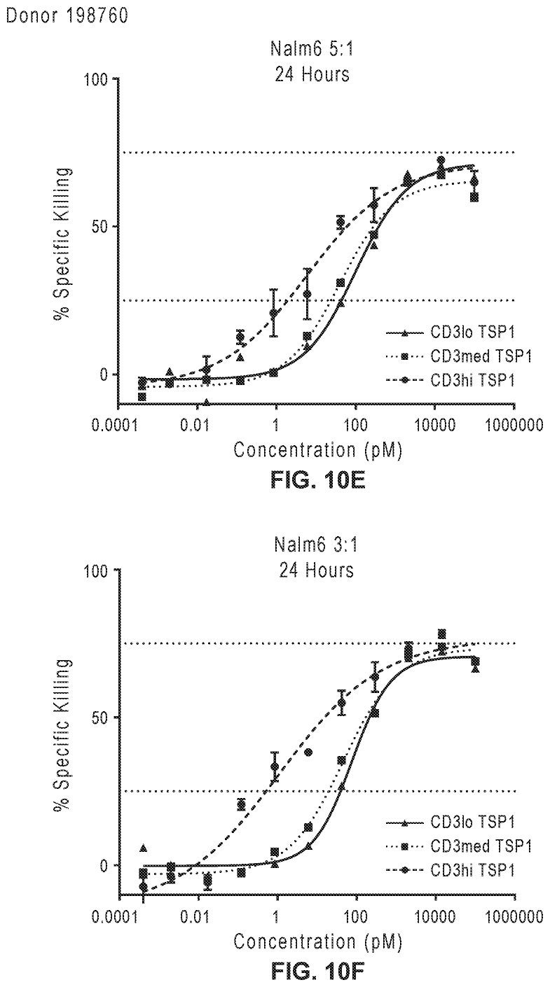

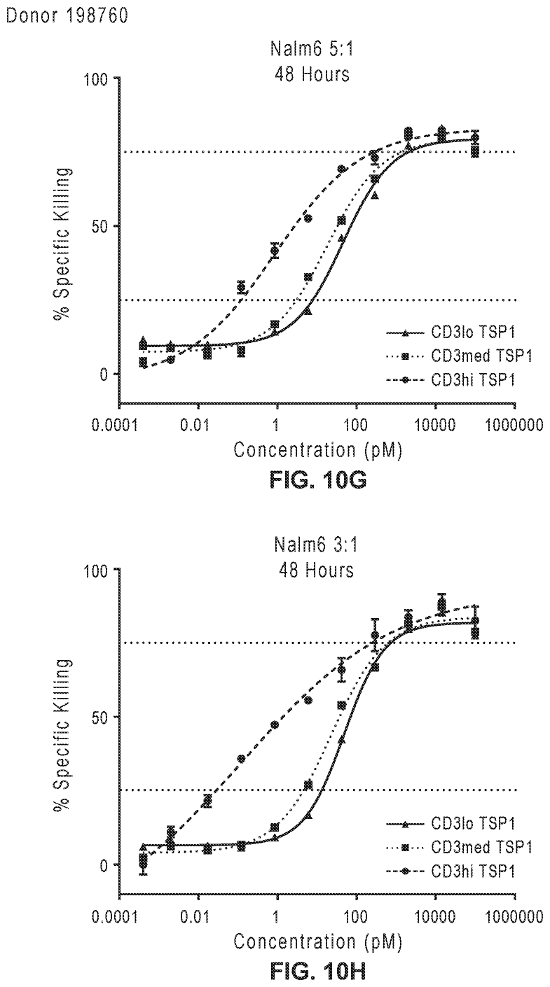

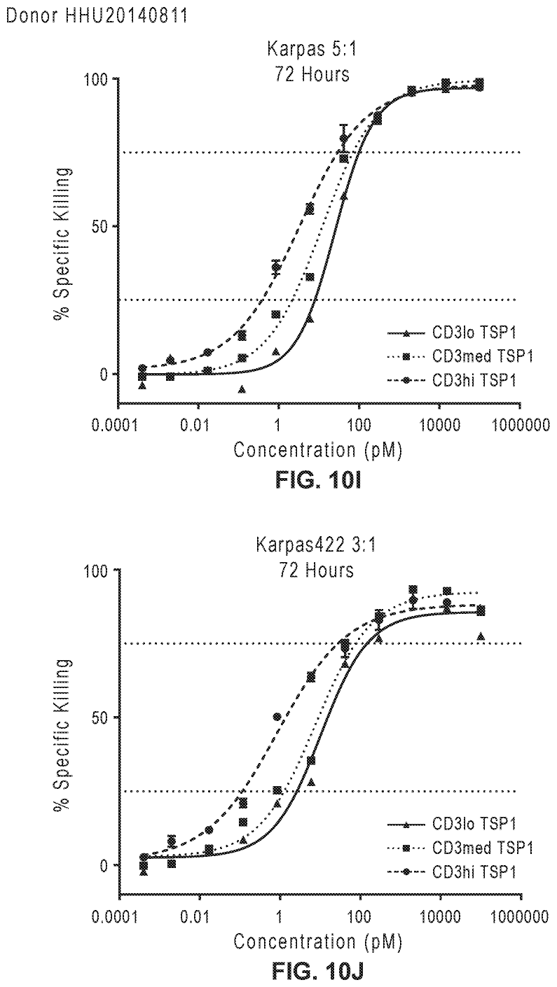

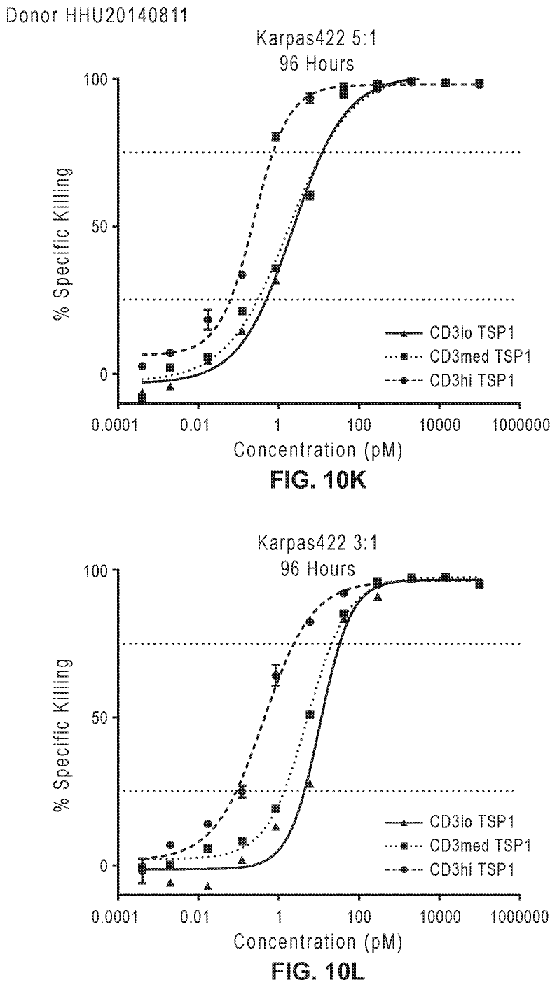

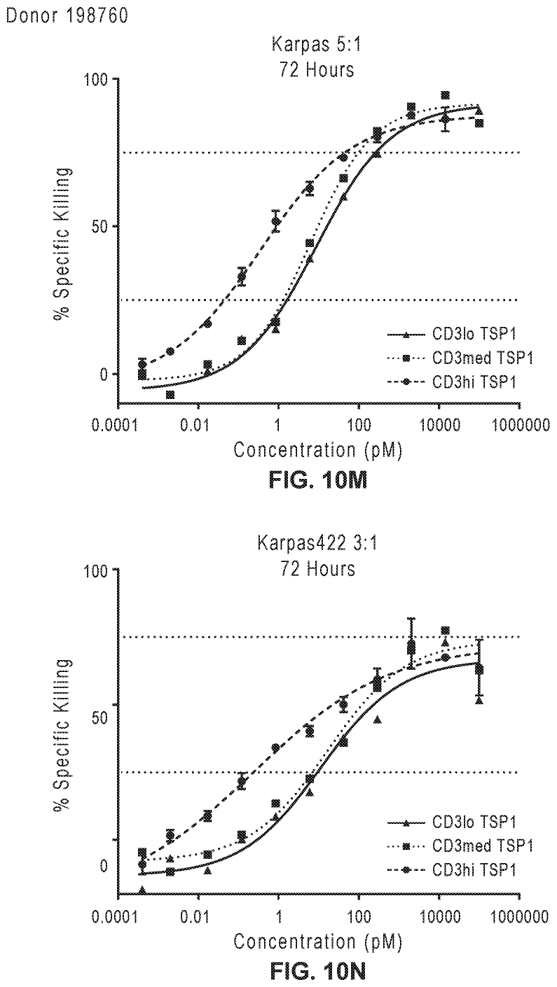

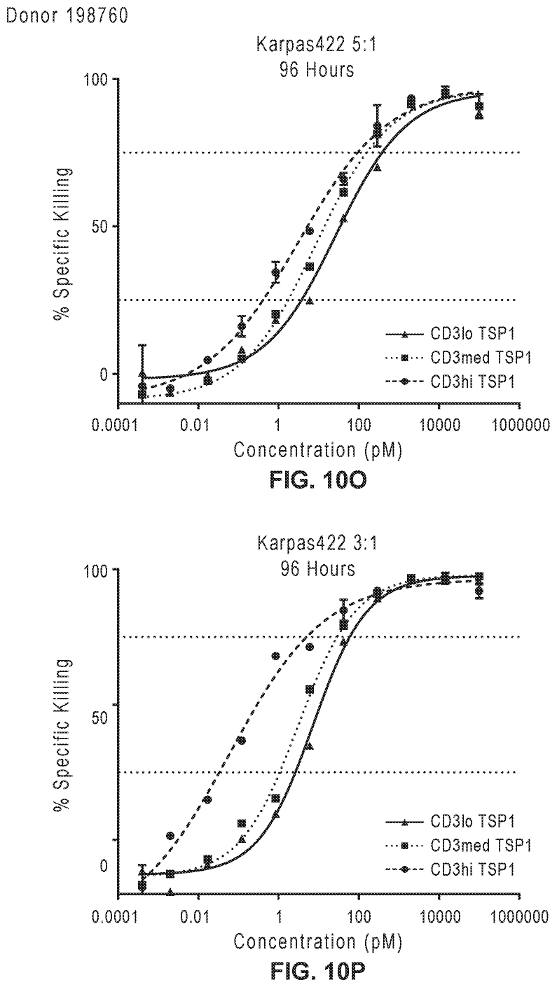

As shown in FIGS. 10A-10P, the TBMs show cytotoxic activity against both Nalm6 target cells (FIGS. 10A-10H) and Karpas422 (FIGS. 101-10P) at multiple time points, E:T ratios and effector T cell donors.

### 8.9. Example 9: RTCC Activity of the NEG258-Based TBMs Vs. BBMs and TBMs that do not Bind to CD2

The NEG258- based TBMs of Example 1A (i.e., R variants) containing either a CD2 binding arm or a control lysozyme binding arm were compared for their potential to induce T cell-mediated apoptosis in Nalm6 or Karpas422 target cells target cells. The study also included blinatumomab as a control. Blinatumomab is a bispecific T cell engager, or BiTE, that binds to both CD19 and CD3 but lacks an Fc domain (see, e.g., U.S. Pat. No. 10,191,034).

### 8.9.1. Materials and Methods

The purified TBMs were compared across multiple donor effector cells. Briefly, huCD19-expressing Nalm6 and Karpas422 target cells were engineered to overexpress firefly luciferase. Cells were harvested and resuspendend in RPMI medium (Invitrogen #11875-093) with 10% FBS. 5,000 target cells per well were plated in a flat-bottom 384-well plate. Human pan T effector cells were isolated via negative selection (Stemcell Technologies #17951) from two donors from cryopreserved PBMCs that were separated from a leukopak (Hemacare #PB001F-1) by Ficoll density gradient centrifugation. Purified T cells were then added to the plate to obtain a final E:T ratio of 3:1, 1:1, 1:3 or 1:5. Co-cultured cells were incubated with serial dilutions of all constructs and controls. For normalization, average maximum luminescence refers to target cells co-incubated with effector cells, but without any test construct. After an incubation of 48, 72 or 96 hr at 37° C., 5% C02, OneGlo luciferase substrate (Promega #E6120) was added to the plate. Luminescence was measured on an Envision plate reader after a 10 minute incubation. Percent specific lysis was calculated using the following equation: Specific lysis (%)=(1−(sample luminescence/average maximum luminescence))*100

### 8.9.2. Results

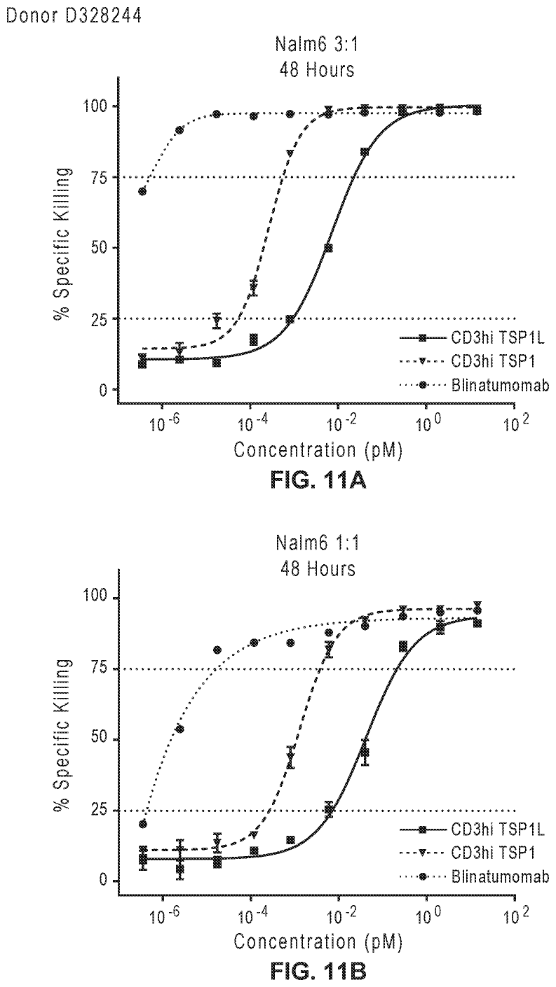

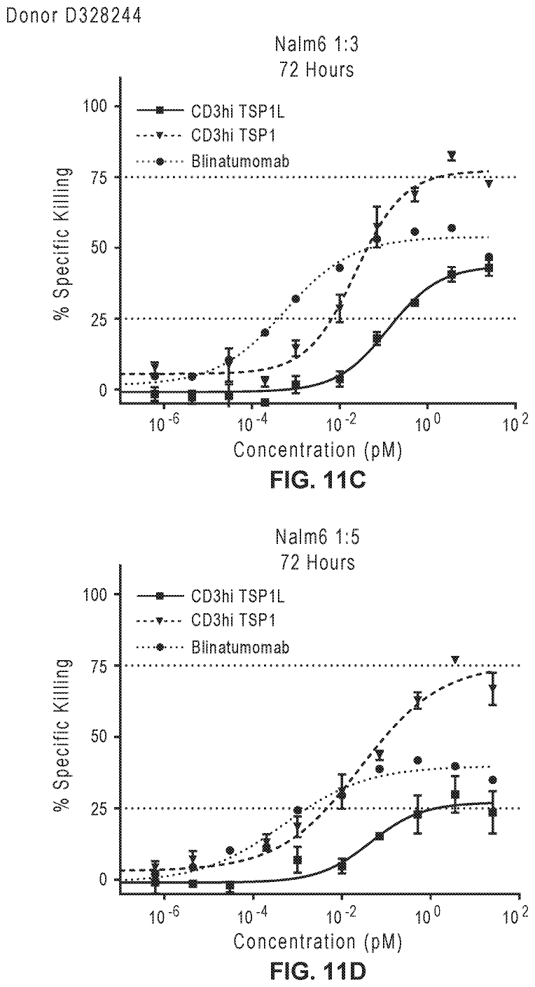

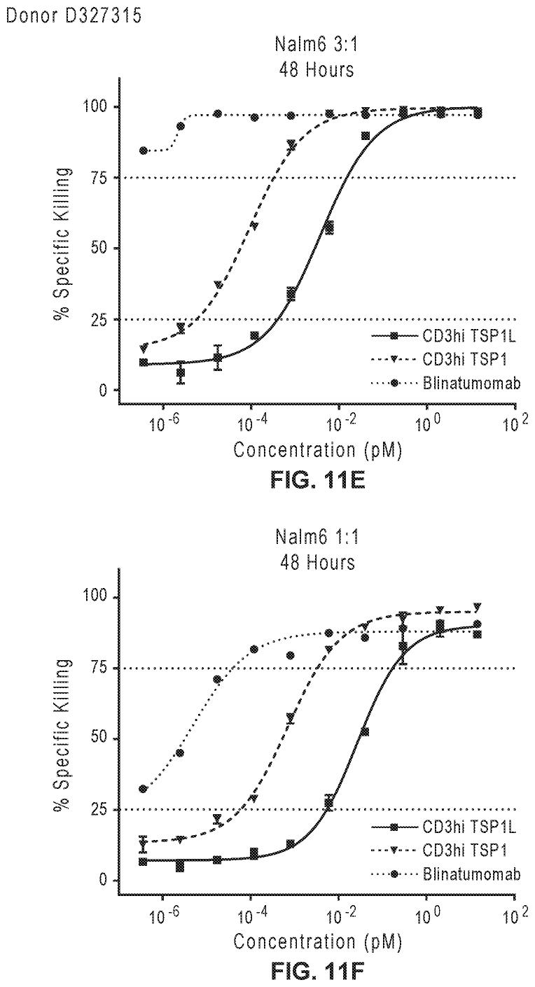

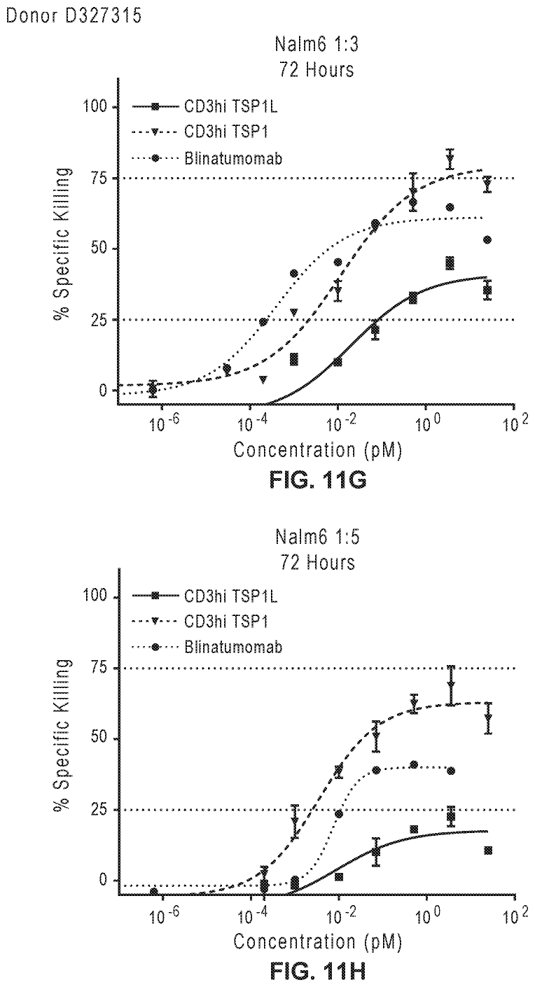

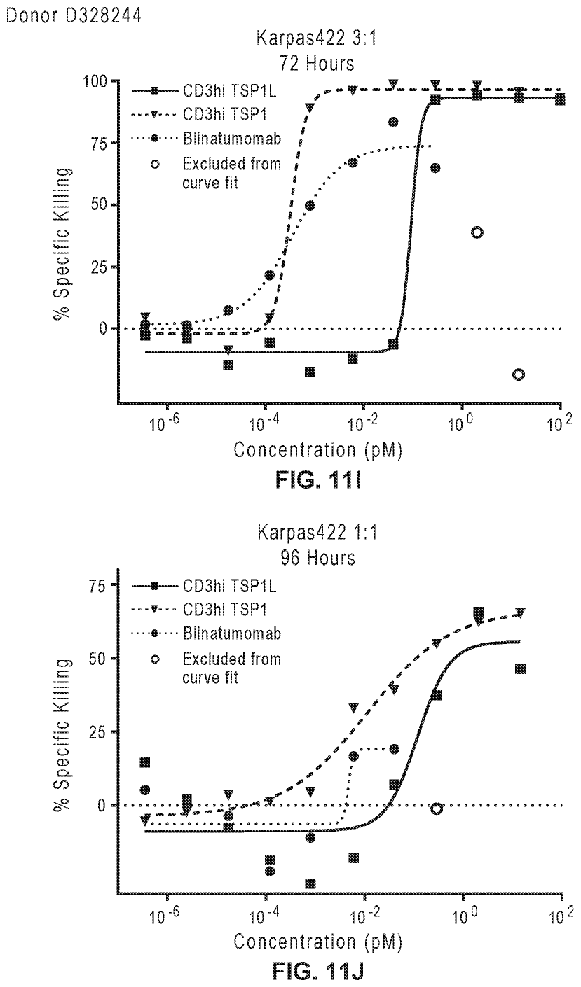

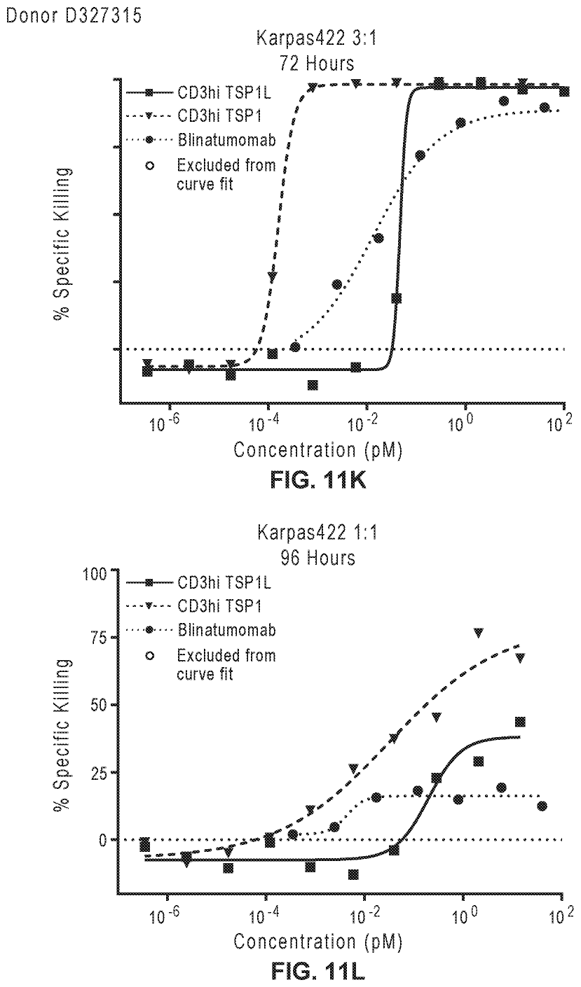

As shown in FIGS. 11A-11L, both types of TBMs show cytotoxic activity against both Nalm6 target cells (FIGS. 11A-11H) and Karpas422 cells (FIGS. 11I-11L). The TBM containing a CD2 binding arm demonstrated superior cytotoxic activity compared to the control TBM with a lysozyme binding arm and to blinatumomab, particularly at lower E:T ratios.

### 8.10. Example 10: Cytokine Release Assay

NEG258- and NEG218-based TBMs of Example 1A (i.e., R variants) were analyzed for their ability to induce T cell-mediated de novo secretion of cytokines in the presence of tumor target cells.

### 8.10.1. Materials and Methods

Briefly, huCD19-expressing Nalm6 target cells were harvested and resuspended in RPMI medium with 10% FBS. 20,000 target cells per well were plated in a flat-bottom 96-well plate. Human pan T effector cells were isolated via MACS negative selection from cryopreserved PBMC then added to the plate to obtain a final E:T ratio of 5:1. Co-cultured cells were incubated with serial dilutions of all constructs and controls. After an incubation of 24 hr at 37° C., 5% CO2, the supernatants were harvested by centrifugation at 300×g for 5 min for subsequent analysis.

A multiplexed ELISA was performed according to the manufacturer's instructions using a V-PLEX Proinflammatory Panel 1 Kit (MesoScale Discovery #K15049D).

### 8.10.2. Results

As shown in FIGS. 12A-12C, both NEG258- and NEG218-based TBMs induce significant cytokine secretion by T cells at all dose levels measured. These figures indicate that they can be effective at lower doses.

### 8.11. Example 11: Binding of NEG258- and NEG218-Based TBMs to Human and Cyno CD19

### 8.11.1. Materials and Methods

The mouse cell line 300.19 was engineered to overexpress either human CD19 or cyno CD19. Cells were cultured in in RPMI medium (Invitrogen #11875-093) with 10% FBS and 2-mercaptoethanol. Cells were harvested and resuspended in FACS buffer (PBS containing 1% FBS). 50,000 cells per well were plated in a V-bottom 96-well plate. Each cell line was incubated with serial dilutions of TBMs of Example 1A (i.e., R variants) for one hour on ice. Cells were centrifuged for 4 min at 400×g and washed with FACS buffer. This was repeated twice, and then the cells were incubated with Alexa-647 labeled anti-human Fc secondary antibody (Jackson Immuno #109-605-098) for 30 min on ice. The cells were washed twice, then resuspended in 100 μL of FACS buffer. FACS data was collected on a Beckman Coulter Cytoflex and analysis was performed using CytExpert v2.3.

### 8.11.2. Results

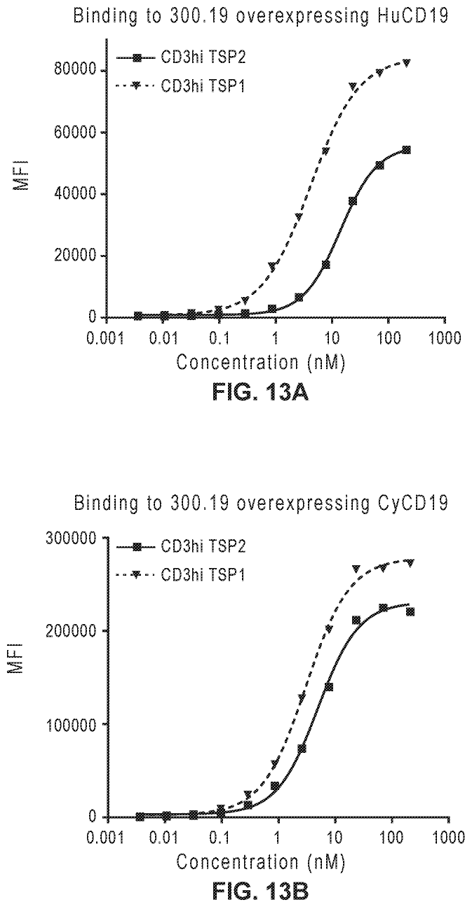

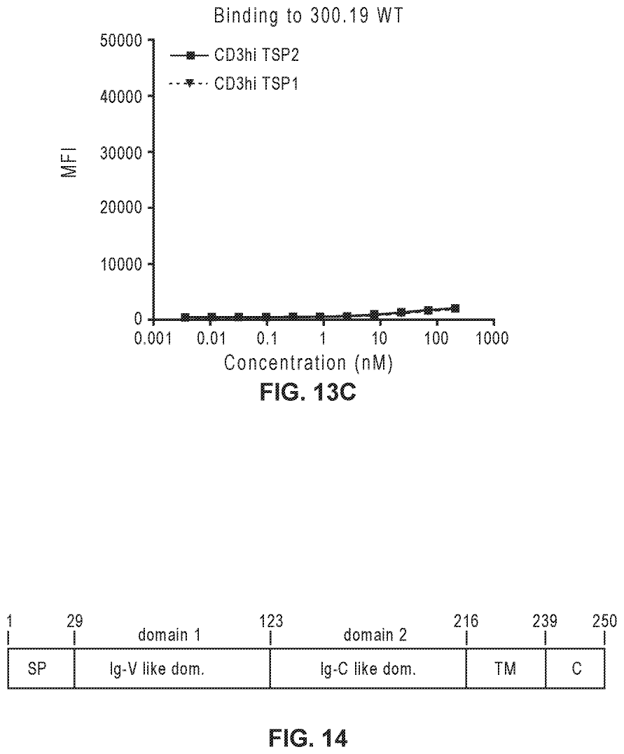

As shown in FIGS. 13A-13B, the NEG258- and NEG218-based TBMs bind to cell lines engineered to overexpress both human and cyno CD19. NEG258 appears to bind equally to both human and cyno while NEG218 appears to have greater affinity for cyno CD19 than human CD19. Of the two, NEG258 appears to have greater affinity for both human CD19 and cyno CD19.

### 8.12. Example 12: Engineering CD58 for Improved Stability

### 8.12.1. Background

Human CD58 contains a signal peptide of 29 amino acids and two Ig-like domains. The most N-terminal Ig-like domain, referred to as domain 1, is of V-type, similar to a variable region of an antibody, and the second domain, named domain 2, is of C-type, is similar to a constant regions of an antibody. A schematic overview of the CD58 domain structure is shown in FIG. 14.

As illustrated in Examples 1-11, domain 1 of CD58, which interacts with CD2, can be used in lieu of an anti-CD2 antibody binding fragment in multispecific binding molecules. The use of a CD58 binding arm rather than an anti-CD2 binding arm reduces non-specific immune activation in the absence of target cells. However, CD58 exhibits lower stability than immunoglobulins.

In order to improve stability of human CD58 domain 1, the protein was engineered to include a pair of cysteine that form a disulfide bridge upon expression to stabilize the molecule.

Four different pairs of amino acids were engineered to be replaced by cysteines: (1) V45 and M105, (2) V45 and M114, (3) V54 and G88 and (4) W56 and L90.

### 8.12.2. Materials and Methods

### 8.12.2.1. Recombinant Expression

To assess the binding and biophysical characteristics, the CD58 disulfide variants were transiently produced and purified from HEK293 cells along with the CD2 extracellular domain. All plasmids were codon optimized for mammalian expression. Human and cyno CD2 constructs were produced with a C-terminal Avi-Tag and a N terminal 8×his tag (SEQ ID NO: 769) followed by a EVNLYFQS sequence (SEQ ID NO: 770) for cleavage of the histag after purification. CD2 constructs were site selectively biotinylated during expression via co-transfection of a plasmid encoding the BirA enzyme. CD58 was expressed with a C-terminal 8×his tag (SEQ ID NO: 769). Transient expression and purification in HEK293F cells was performed with standard methodology. The sequences are shown in Table 21.

TABLE 21

| Pro- | | SEQ |
|---|---|---|
| tein | | ID |
| Name | AA Sequence | NO: |
| Human | SKEITNALETWGALGQDINLDIPSFQMSDDIDDIKWEKTS | 1321 |
| CD2 | DKKKIAQFRKEKETFKEKDTYKLFKNGTLKIKHLKTDDQ | |
| | DIYKVSIYDTKGKNVLEKIFDLKIQERVSKPKISWTCINT | |
| | TLTCEVMNGTDPELNLYQDGKHLKLSQRVITHKWTTSLS | |
| | AKFKCTAGNKVSKESSVEPVSCPEKGLDGGGGSGLNDI | |
| | FEAQKIEWHE | |
| Cyno | SKEIRNALETWGALGQDIDLDIPSFQMSDDIDDIRWEKT | 1322 |
| CD2 | SDKKKIAQFRKEKETFEEKDAYKLFKNGTLKIKHLKIHDQ | |
| | DSYKVSIYDTKGKNVLEKTFDLKIQERVSEPKISWTCINT | |
| | TLTCEVMNGTDPELNLYQDGKHVKLSQRVITHKWTTSL | |
| | SAKFKCTAGNKVSKESRMETVSCPEKGLDGGGGSGLN | |
| | DIFEAQKIEWHE | |
| CD58 | SQQIYGVVYGNVTFHVPSNVPLKEVLWKKQKDKVAELE | 1323 |
| Full | NSEFRAFSSFKNRVYLDTVSGSLTIYNLTSSDEDEYEME | |
| ECD | SPNITDTMKFFLYVLESLPSPTLTCALTNGSIEVQCMIPE | |
| | HYNSHRGLIMYSWDCPMEQCKRNSTSIYFKMENDLPQ | |
| | KIQCTLSNPLFNTTSSIILTTCIPSSGHSRHRGGGGSHHH | |
| | HHHHH | |
| CD58_ | SQQIYGVVYGNVTFHVPSNVPLKEVLWKKQKDKVAELE | 1324 |
| IgV | NSEFRAFSSFKNRVYLDTVSGSLTIYNLTSSDEDEYEME | |
| | SPNITDTMKFFLYVLESGGGGSHHHHHHHH | |
| IgV | SQQIYGVVYGNVTFHCPSNVPLKEVLWKKQKDKVAELE | 1325 |
| V45C_ | NSEFRAFSSFKNRVYLDTVSGSLTIYNLTSSDEDEYECE | |
| M105C | SPNITDTMKFFLYVLESGGGGSHHHHHHHH | |
| IgV | SQQIYGVVYGNVTFHVPSNVPLKECLWKKQKDKVAELE | 1326 |
| V54C_ | NSEFRAFSSFKNRVYLDTVSCSLTIYNLTSSDEDEYEME | |
| G88C | SPNITDTMKFFLYVLESGGGGSHHHHHHHH | |
| IgV | SQQIYGVVYGNVTFHCPSNVPLKEVLWKKQKDKVAELE | 1327 |
| V45C_ | NSEFRAFSSFKNRVYLDTVSGSLTIYNLTSSDEDEYEME | |
| M114C | SPNITDTCKFFLYVLESGGGGSHHHHHHHH | |

For expression, transfection was performed using PEI as transfection reagent. For small scale (<5L) transfections, cells were grown in shake flasks on an orbital shaker (100 rpm) in a humidified incubator (85%) at 8% 002). Transfection was done with a ratio of 1 DNA:3 PEI. 1 mg/L culture of plasmid was used for transfection at 2.0 million cells/mL in Expi293 medium. After 5 days of expression, the culture was centrifuged and filtrated. Purification was performed via Nickel-NTA batch binding using 1 ml resin/100 mL supernatant. The protein was allowed to bind for a minimum of 2 hours with gentle mixing, and the mixture was loaded onto a gravity filtration column. The resin was washed with 30 CV of PBS. Proteins were eluted with imidazole. The eluted protein was concentrated and finally purified via a preparative size exclusion chromatography (Hi Load 16/60 Superdex 75 grade column, GE Healthcare Life Sciences, Uppsala, Sweden). To confirm that the identity of the proteins expressed matched the predicted masses for the primary amino acid sequences, proteins were analyzed by high-performance liquid chromatography coupled to mass spectrometry.

### 8.12.2.2. Stability

Disulfide stabilized variants were assessed for improved thermal stability using both differential scanning calorimetry (DSC) and differential scanning fluorimetry (DSF) using standard techniques. For DSF, 1-3 ug of each construct was add to 1× Sypro Orange (Thermo-Fisher) in 25ul total volume in 96-well PCR plate. Using a Bio-Rad CFX96 RT-PCR system equipped with C1000 Thermal Cycler, the temperature was increased from 25° C. to 95° C. at 0.5° C./minute and the fluorescence monitored. The manufacturer-supplied software was used to determine Tm.

For DSC, all samples were dialyzed into HEPES-buffered saline (HBS) and diluted to final concentration of 0.5 mg/mL. Tm and Tonset were determined using a MicroCal VP-Capillary DSC system (Malvern) by increasing temperature from 25° C. to 100° C. at 1° C./minute with a filtering period of 2 seconds and a mid-gain setting.

### 8.12.2.3. Binding Affinity

To ensure the binding affinity remained uncompromised by the additional of the stabilizing disulfide variance, isothermal calorimetry (ITC) was performed on the resulting recombinant CD58 proteins to determine their apparent KD and binding stoicheometry (n) to recombinant human CD2.

Briefly, recombinant human CD2 and recombinant human CD58 variants were dialyzed into HEPES-buffered saline (HBS). CD2 was diluted to final concentration of 100 μM, CD58 variants were diluted to 10 μM. CD2 was titrated into 10 μM of CD58 variants via multiple injections and ΔH (kcal/mole) determined using a MicroCal VP-ITC isothermal titration calorimeter (Malvern). Titrations of CD2 into HBS were used as a reference and KD and n determined from the resulting data.

### 8.12.3. Results

Results for both DSF and DSC measurements for the constructs are shown in Table 22 below.

TABLE 22

| | By Differential | | |
|---|---|---|---|
| | Scanning | By Differential Scanning | |
| | Fluorimetry (DSF) | Calorimetry (DSC) | |
| CD58 variant | Tm (° C.) | Tmonset (° C.) | Tm (° C.) |
| CD58 Full ECD | 59.5 | 48.8 | 65.0 |
| CD58_IgV | 48.5 | 46.3 | 60.9 |
| IgV V45C_M105C | 48.5 | 43.9 | 66.8 |
| IgV V54C_G88C | 76.5 | 66.7 | 80.9 |
| IgV V45C_M114C | 63.5 | 49.6 | 72.5 |

Results of the affinity studies are shown in Table 23 below. Addition of stabilizing disulfide had no detrimental impact on the affinity or the binding stoicheometry.

TABLE 23

| CD58 variant | KD (uM) | n |
|---|---|---|
| CD58 Full ECD | 0.57 (±0.05) | 0.92 (±0.01) |
| CD58_IgV | 0.61 (±0.07) | 0.96 (±0.01) |
| IgV V45C_M105C | 0.88 (±0.06) | 0.97 (±0.01) |
| IgV V54C_G88C | 0.60 (±0.06) | 0.83 (±.0.01) |
| IgV V45C_M114C | 0.38 (±0.03) | 0.88 (±.0.01) |

### 8.13. Example 13: Production of Anti-CD3-Anti-CD19-CD58 IgG1 TBMs in Knob-into-Holes Format

### 8.13.1. Materials and Methods

Constructs were synthesized and codon optimized for expression in mammalian cells. For each trispecific construct, three plasmids were synthesized. A first plasmid encoding an anti-CD19 heavy chain was synthesized as a fusion comprising (in the N-terminal to C-terminal direction) (i) a VH domain fused to a constant hIgG1 CH1 domain, (ii) a linker, (iii) an anti-CD3 scFv, (iv) a second linker and (v) a hIgG1 Fc domain containing mutations for a hole to facilitate heterodimerization as well as silencing mutations. A second plasmid encoding a light chain was synthesized as a fusion comprising (in the N-terminal to C-terminal direction) an anti-CD19 VL domain and (ii) a constant human kappa sequence. A third plasmid encoding a second half antibody was synthesized as a fusion comprising (in the N-terminal to C-terminal direction) a CD58 disulfide stabilized variant fused to a constant hIgG1 domain containing mutations for a knob to facilitate heterodimerization as well as silencing mutations. The sequences are shown in Table 24.

TABLE 24

| | | | SEQ |
|---|---|---|---|
| Trispecific | Chain | | ID |
| Name | Description | Sequence | NO: |
| CD19_CTL119 | First Half | QVQLQESGPGLVKPSETLSLTCTVSGVSLPDYG | 1328 |
| _CD3_16nM- | Antibody | VSWIRQPPGKGLEWIGVIWGSETTYYQSSLKSR | |
| CD58_Full | Heavy Chain | VTISKDNSKNQVSLKLSSVTAADTAVYYCAKHY | |
| ECD | (Fc | YYGGSYAMDYWGQGTLVTVSSASTKGPSVFPL | |
| Trispecific | sequence | APSSKSTSGGTAALGCLVKDYFPEPVTVSWNS | |
| | not shown) | GALTSGVHTFPAVLQSSGLYSLSSVVTVPSSSL | |
| | | GTQTYICNVNHKPSNTKVDKRVEPKSCGGGGS | |
| | | GGGGSEVQLVESGGGLVQPGGSLKLSCAASGF | |
| | | TFNTYAMNWVRQASGKGLEWVGRIRSKYNNYA | |
| | | TYYADSVKDRFTISRDDSKSTLYLQMNSLKTED | |
| | | TAVYYCVRHGNFGNSYVSWFAYWGQGTLVTVS | |
| | | SGGGGSGGGGSGGGGSGGGGSQAVVTQEPS | |
| | | LTVSPGGTVTLTCRSSTGAVTTSNYANWVQQK | |
| | | PGQAPRGLIGGTNKRAPWTPARFSGSLLGDKA | |
| | | ALTLSGAQPEDEAEYFCALWYSNLWVFGGGTK | |
| | | LTVLGGGGS | |
| | First Half | QVQLQESGPGLVKPSETLSLTCTVSGVSLPDYG | 1094 |
| | Antibody | VSWIRQPPGKGLEWIGVIWGSETTYYQSSLKSR | |
| | Heavy Chain | VTISKDNSKNQVSLKLSSVTAADTAVYYCAKHY | |
| | (includes Fc | YYGGSYAMDYWGQGTLVTVSSASTKGPSVFPL | |
| | sequence) | APSSKSTSGGTAALGCLVKDYFPEPVTVSWNS | |
| | | GALTSGVHTFPAVLQSSGLYSLSSVVTVPSSSL | |
| | | GTQTYICNVNHKPSNTKVDKRVEPKSCGGGGS | |
| | | GGGGSEVQLVESGGGLVQPGGSLKLSCAASGF | |
| | | TFNTYAMNWVRQASGKGLEWVGRIRSKYNNYA | |
| | | TYYADSVKDRFTISRDDSKSTLYLQMNSLKTED | |
| | | TAVYYCVRHGNFGNSYVSWFAYWGQGTLVTVS | |
| | | SGGGGSGGGGSGGGGSGGGGSQAVVTQEPS | |
| | | LTVSPGGTVTLTCRSSTGAVTTSNYANWVQQK | |
| | | PGQAPRGLIGGTNKRAPWTPARFSGSLLGDKA | |
| | | ALTLSGAQPEDEAEYFCALWYSNLWVFGGGTK | |
| | | LTVLGGGGSDKTHTCPPCPAPELLGGPSVFLFP | |
| | | PKPKDTLMISRTPEVTCVVVAVSHEDPEVKFNW | |
| | | YVDGVEVHNAKTKPREEQYGSTYRVVSVLTVLH | |
| | | QDWLNGKEYKCKVSNKALAAPIEKTISKAKGQP | |
| | | REPQVCTLPPSRDELTKNQVSLSCAVKGFYPSD | |
| | | IAVEWESNGQPENNYKTTPPVLDSDGSFFLVSK | |
| | | LTVDKSRWQQGNVFSCSVMHEALHNHYTQKSL | |
| | | SLSPGK | |
| | Second Half | FSQQIYGVVYGNVTFHVPSNVPLKEVLWKKQKD | 1329 |
| | Antibody | KVAELENSEFRAFSSFKNRVYLDTVSGSLTIYNL | |
| | (Fc | TSSDEDEYEMESPNITDTMKFFLYVLESLPSPTL | |
| | sequence | TCALTNGSIEVQCMIPEHYNSHRGLIMYSWDCP | |
| | not shown) | MEQCKRNSTSIYFKMENDLPQKIQCTLSNPLFN | |
| | | TTSSIILTTCIPSSGHSRHRGGGS | |
| | Second Half | FSQQIYGVVYGNVTFHVPSNVPLKEVLWKKQKD | 1330 |
| | Antibody | KVAELENSEFRAFSSFKNRVYLDTVSGSLTIYNL | |
| | (includes Fc | TSSDEDEYEMESPNITDTMKFFLYVLESLPSPTL | |
| | sequence) | TCALTNGSIEVQCMIPEHYNSHRGLIMYSWDCP | |
| | | MEQCKRNSTSIYFKMENDLPQKIQCTLSNPLFN | |
| | | TTSSIILTTCIPSSGHSRHRGGGSDKTHTCPPCP | |
| | | APELLGGPSVFLFPPKPKDTLMISRTPEVTCVVV | |
| | | DVSHEDPEVKFNWYVDGVEVHNAKTKPREEQY | |
| | | ASTYRVVSVLTVLHQDWLNGKEYKCKVSNKALP | |
| | | APIEKTISKAKGQPREPQVYTLPPCREEMTKNQ | |
| | | VSLWCLVKGFYPSDIAVEWESNGQPENNYKTT | |
| | | PPVLDSDGSFFLYSKLTVDKSRWQQGNVFSCS | |
| | | VMHEALHNRYTQKSLSLSPGK | |
| | First Half | EIVMTQSPATLSLSPGERATLSCRASQDISKYLN | 1331 |
| | Antibody | WYQQKPGQAPRLLIYHTSRLHSGIPARFSGSGS | |
| | Light Chain | GTDYTLTISSLQPEDFAVYFCQQGNTLPYTFGQ | |
| | | GTKLEIKRTVAAPSVFIFPPSDEQLKSGTASVVC | |
| | | LLNNFYPREAKVQWKVDNALQSGNSQESVTEQ | |
| | | DSKDSTYSLSSTLTLSKADYEKHKVYACEVTHQ | |
| | | GLSSPVTKSFNRGEC | |
| CD19_CTL119 | First Half | QVQLQESGPGLVKPSETLSLTCTVSGVSLPDYG | 1328 |
| _CD3_16nM- | Antibody | VSWIRQPPGKGLEWIGVIWGSETTYYQSSLKSR | |
| CD58_IgV | Heavy Chain | VTISKDNSKNQVSLKLSSVTAADTAVYYCAKHY | |
| Trispecific | (Fc | YYGGSYAMDYWGQGTLVTVSSASTKGPSVFPL | |
| | sequence | APSSKSTSGGTAALGCLVKDYFPEPVTVSWNS | |
| | not shown) | GALTSGVHTFPAVLQSSGLYSLSSVVTVPSSSL | |
| | | GTQTYICNVNHKPSNTKVDKRVEPKSCGGGGS | |
| | | GGGGSEVQLVESGGGLVQPGGSLKLSCAASGF | |
| | | TFNTYAMNWVRQASGKGLEWVGRIRSKYNNYA | |
| | | TYYADSVKDRFTISRDDSKSTLYLQMNSLKTED | |
| | | TAVYYCVRHGNFGNSYVSWFAYWGQGTLVTVS | |
| | | SGGGGSGGGGSGGGGSGGGGSQAVVTQEPS | |
| | | LTVSPGGTVTLTCRSSTGAVTTSNYANWVQQK | |
| | | PGQAPRGLIGGTNKRAPWTPARFSGSLLGDKA | |
| | | ALTLSGAQPEDEAEYFCALWYSNLWVFGGGTK | |
| | | LTVLGGGGS | |
| | First Half | QVQLQESGPGLVKPSETLSLTCTVSGVSLPDYG | 1094 |
| | Antibody | VSWIRQPPGKGLEWIGVIWGSETTYYQSSLKSR | |
| | Heavy Chain | VTISKDNSKNQVSLKLSSVTAADTAVYYCAKHY | |
| | (includes Fc | YYGGSYAMDYWGQGTLVTVSSASTKGPSVFPL | |
| | sequence) | APSSKSTSGGTAALGCLVKDYFPEPVTVSWNS | |
| | | GALTSGVHTFPAVLQSSGLYSLSSVVTVPSSSL | |
| | | GTQTYICNVNHKPSNTKVDKRVEPKSCGGGGS | |
| | | GGGGSEVQLVESGGGLVQPGGSLKLSCAASGF | |
| | | TFNTYAMNWVRQASGKGLEWVGRIRSKYNNYA | |
| | | TYYADSVKDRFTISRDDSKSTLYLQMNSLKTED | |
| | | TAVYYCVRHGNFGNSYVSWFAYWGQGTLVTVS | |
| | | SGGGGSGGGGSGGGGSGGGGSQAVVTQEPS | |
| | | LTVSPGGTVTLTCRSSTGAVTTSNYANWVQQK | |
| | | PGQAPRGLIGGTNKRAPWTPARFSGSLLGDKA | |
| | | ALTLSGAQPEDEAEYFCALWYSNLWVFGGGTK | |
| | | LTVLGGGGSDKTHTCPPCPAPELLGGPSVFLFP | |
| | | PKPKDTLMISRTPEVTCVVVAVSHEDPEVKFNW | |
| | | YVDGVEVHNAKTKPREEQYGSTYRVVSVLTVLH | |
| | | QDWLNGKEYKCKVSNKALAAPIEKTISKAKGQP | |
| | | REPQVCTLPPSRDELTKNQVSLSCAVKGFYPSD | |
| | | IAVEWESNGQPENNYKTTPPVLDSDGSFFLVSK | |
| | | LTVDKSRWQQGNVFSCSVMHEALHNHYTQKSL | |
| | | SLSPGK | |
| | Second Half | SQQIYGVVYGNVTFHVPSNVPLKEVLWKKQKD | 65 |
| | Antibody | KVAELENSEFRAFSSFKNRVYLDTVSGSLTIYNL | |
| | (Fc | TSSDEDEYEMESPNITDTMKFFLYVLESGGGGS | |
| | sequence | | |
| | not shown) | | |
| | Second Half | SQQIYGVVYGNVTFHVPSNVPLKEVLWKKQKD | 75 |
| | Antibody | KVAELENSEFRAFSSFKNRVYLDTVSGSLTIYNL | |
| | (includes Fc | TSSDEDEYEMESPNITDTMKFFLYVLESGGGGS | |
| | sequence) | DKTHTCPPCPAPELLGGPSVFLFPPKPKDTLMIS | |
| | | RTPEVTCVVVAVSHEDPEVKFNWYVDGVEVHN | |
| | | AKTKPREEQYASTYRVVSVLTVLHQDWLNGKE | |
| | | YKCKVSNKALAAPIEKTISKAKGQPREPQVYTLP | |
| | | PCREEMTKNQVSLWCLVKGFYPSDIAVEWESN | |
| | | GQPENNYKTTPPVLDSDGSFFLYSKLTVDKSR | |
| | | WQQGNVFSCSVMHEALHNRYTQKSLSLSPGK | |
| | First Half | EIVMTQSPATLSLSPGERATLSCRASQDISKYLN | 1331 |
| | Antibody | WYQQKPGQAPRLLIYHTSRLHSGIPARFSGSGS | |
| | Light Chain | GTDYTLTISSLQPEDFAVYFCQQGNTLPYTFGQ | |
| | | GTKLEIKRTVAAPSVFIFPPSDEQLKSGTASVVC | |
| | | LLNNFYPREAKVQWKVDNALQSGNSQESVTEQ | |
| | | DSKDSTYSLSSTLTLSKADYEKHKVYACEVTHQ | |
| | | GLSSPVTKSFNRGEC | |
| CD19_CTL119 | First Half | QVQLQESGPGLVKPSETLSLTCTVSGVSLPDYG | 1328 |
| _CD3_16nM- | Antibody | VSWIRQPPGKGLEWIGVIWGSETTYYQSSLKSR | |
| CD58_IgV_ | Heavy Chain | VTISKDNSKNQVSLKLSSVTAADTAVYYCAKHY | |
| V45C_M105C | (Fc | YYGGSYAMDYWGQGTLVTVSSASTKGPSVFPL | |
| Trispecific | sequence | APSSKSTSGGTAALGCLVKDYFPEPVTVSWNS | |
| | not shown) | GALTSGVHTFPAVLQSSGLYSLSSVVTVPSSSL | |
| | | GTQTYICNVNHKPSNTKVDKRVEPKSCGGGGS | |
| | | GGGGSEVQLVESGGGLVQPGGSLKLSCAASGF | |
| | | TFNTYAMNWVRQASGKGLEWVGRIRSKYNNYA | |
| | | TYYADSVKDRFTISRDDSKSTLYLQMNSLKTED | |
| | | TAVYYCVRHGNFGNSYVSWFAYWGQGTLVTVS | |
| | | SGGGGSGGGGSGGGGSGGGGSQAVVTQEPS | |
| | | LTVSPGGTVTLTCRSSTGAVTTSNYANWVQQK | |
| | | PGQAPRGLIGGTNKRAPWTPARFSGSLLGDKA | |
| | | ALTLSGAQPEDEAEYFCALWYSNLWVFGGGTK | |
| | | LTVLGGGGS | |
| | First Half | QVQLQESGPGLVKPSETLSLTCTVSGVSLPDYG | 1094 |
| | Antibody | VSWIRQPPGKGLEWIGVIWGSETTYYQSSLKSR | |
| | Heavy Chain | VTISKDNSKNQVSLKLSSVTAADTAVYYCAKHY | |
| | (includes Fc | YYGGSYAMDYWGQGTLVTVSSASTKGPSVFPL | |
| | sequence) | APSSKSTSGGTAALGCLVKDYFPEPVTVSWNS | |
| | | GALTSGVHTFPAVLQSSGLYSLSSVVTVPSSSL | |
| | | GTQTYICNVNHKPSNTKVDKRVEPKSCGGGGS | |
| | | GGGGSEVQLVESGGGLVQPGGSLKLSCAASGF | |
| | | TFNTYAMNWVRQASGKGLEWVGRIRSKYNNYA | |
| | | TYYADSVKDRFTISRDDSKSTLYLQMNSLKTED | |
| | | TAVYYCVRHGNFGNSYVSWFAYWGQGTLVTVS | |
| | | SGGGGSGGGGSGGGGSGGGGSQAVVTQEPS | |
| | | LTVSPGGTVTLTCRSSTGAVTTSNYANWVQQK | |
| | | PGQAPRGLIGGTNKRAPWTPARFSGSLLGDKA | |
| | | ALTLSGAQPEDEAEYFCALWYSNLWVFGGGTK | |
| | | LTVLGGGGSDKTHTCPPCPAPELLGGPSVFLFP | |
| | | PKPKDTLMISRTPEVTCVVVAVSHEDPEVKFNW | |
| | | YVDGVEVHNAKTKPREEQYGSTYRVVSVLTVLH | |
| | | QDWLNGKEYKCKVSNKALAAPIEKTISKAKGQP | |
| | | REPQVCTLPPSRDELTKNQVSLSCAVKGFYPSD | |
| | | IAVEWESNGQPENNYKTTPPVLDSDGSFFLVSK | |
| | | LTVDKSRWQQGNVFSCSVMHEALHNHYTQKSL | |
| | | SLSPGK | |
| | Second Half | SQQIYGVVYGNVTFHCPSNVPLKEVLWKKQKD | 1332 |
| | Antibody | KVAELENSEFRAFSSFKNRVYLDTVSGSLTIYNL | |
| | (Fc | TSSDEDEYECESPNITDTMKFFLYVLESGS | |
| | sequence | | |
| | not shown) | | |
| | Second Half | SQQIYGVVYGNVTFHCPSNVPLKEVLWKKQKD | 1333 |
| | Antibody | KVAELENSEFRAFSSFKNRVYLDTVSGSLTIYNL | |
| | (includes Fc | TSSDEDEYECESPNITDTMKFFLYVLESGSDKT | |
| | sequence) | HTCPPCPAPELLGGPSVFLFPPKPKDTLMISRTP | |
| | | EVTCVVVDVSHEDPEVKFNWYVDGVEVHNAKT | |
| | | KPREEQYASTYRVVSVLTVLHQDWLNGKEYKC | |
| | | KVSNKALPAPIEKTISKAKGQPREPQVYTLPPCR | |
| | | EEMTKNQVSLWCLVKGFYPSDIAVEWESNGQP | |
| | | ENNYKTTPPVLDSDGSFFLYSKLTVDKSRWQQ | |
| | | GNVFSCSVMHEALHNRYTQKSLSLSPGK | |
| | First Half | EIVMTQSPATLSLSPGERATLSCRASQDISKYLN | 1331 |
| | Antibody | WYQQKPGQAPRLLIYHTSRLHSGIPARFSGSGS | |
| | Light Chain | GTDYTLTISSLQPEDFAVYFCQQGNTLPYTFGQ | |
| | | GTKLEIKRTVAAPSVFIFPPSDEQLKSGTASVVC | |
| | | LLNNFYPREAKVQWKVDNALQSGNSQESVTEQ | |
| | | DSKDSTYSLSSTLTLSKADYEKHKVYACEVTHQ | |
| | | GLSSPVTKSFNRGEC | |
| CD19_CTL119 | First Half | QVQLQESGPGLVKPSETLSLTCTVSGVSLPDYG | 1328 |
| _CD3_16nM- | Antibody | VSWIRQPPGKGLEWIGVIWGSETTYYQSSLKSR | |
| CD58_IgV | Heavy Chain | VTISKDNSKNQVSLKLSSVTAADTAVYYCAKHY | |
| V54C_G88C | (Fc | YYGGSYAMDYWGQGTLVTVSSASTKGPSVFPL | |
| Trispecific | sequence | APSSKSTSGGTAALGCLVKDYFPEPVTVSWNS | |
| | not shown) | GALTSGVHTFPAVLQSSGLYSLSSVVTVPSSSL | |
| | | GTQTYICNVNHKPSNTKVDKRVEPKSCGGGGS | |
| | | GGGGSEVQLVESGGGLVQPGGSLKLSCAASGF | |
| | | TFNTYAMNWVRQASGKGLEWVGRIRSKYNNYA | |
| | | TYYADSVKDRFTISRDDSKSTLYLQMNSLKTED | |
| | | TAVYYCVRHGNFGNSYVSWFAYWGQGTLVTVS | |
| | | SGGGGSGGGGSGGGGSGGGGSQAVVTQEPS | |
| | | LTVSPGGTVTLTCRSSTGAVTTSNYANWVQQK | |
| | | PGQAPRGLIGGTNKRAPWTPARFSGSLLGDKA | |
| | | ALTLSGAQPEDEAEYFCALWYSNLWVFGGGTK | |
| | | LTVLGGGGS | |
| | First Half | QVQLQESGPGLVKPSETLSLTCTVSGVSLPDYG | 1094 |
| | Antibody | VSWIRQPPGKGLEWIGVIWGSETTYYQSSLKSR | |
| | Heavy Chain | VTISKDNSKNQVSLKLSSVTAADTAVYYCAKHY | |
| | (includes Fc | YYGGSYAMDYWGQGTLVTVSSASTKGPSVFPL | |
| | sequence) | APSSKSTSGGTAALGCLVKDYFPEPVTVSWNS | |
| | | GALTSGVHTFPAVLQSSGLYSLSSVVTVPSSSL | |
| | | GTQTYICNVNHKPSNTKVDKRVEPKSCGGGGS | |
| | | GGGGSEVQLVESGGGLVQPGGSLKLSCAASGF | |
| | | TFNTYAMNWVRQASGKGLEWVGRIRSKYNNYA | |
| | | TYYADSVKDRFTISRDDSKSTLYLQMNSLKTED | |
| | | TAVYYCVRHGNFGNSYVSWFAYWGQGTLVTVS | |
| | | SGGGGSGGGGSGGGGSGGGGSQAVVTQEPS | |
| | | LTVSPGGTVTLTCRSSTGAVTTSNYANWVQQK | |
| | | PGQAPRGLIGGTNKRAPWTPARFSGSLLGDKA | |
| | | ALTLSGAQPEDEAEYFCALWYSNLWVFGGGTK | |
| | | LTVLGGGGSDKTHTCPPCPAPELLGGPSVFLFP | |
| | | PKPKDTLMISRTPEVTCVVVAVSHEDPEVKFNW | |
| | | YVDGVEVHNAKTKPREEQYGSTYRVVSVLTVLH | |
| | | QDWLNGKEYKCKVSNKALAAPIEKTISKAKGQP | |
| | | REPQVCTLPPSRDELTKNQVSLSCAVKGFYPSD | |
| | | IAVEWESNGQPENNYKTTPPVLDSDGSFFLVSK | |
| | | LTVDKSRWQQGNVFSCSVMHEALHNHYTQKSL | |
| | | SLSPGK | |
| | Second Half | SQQIYGVVYGNVTFHVPSNVPLKECLWKKQKD | 1334 |
| | Antibody | KVAELENSEFRAFSSFKNRVYLDTVSCSLTIYNL | |
| | (Fc | TSSDEDEYEMESPNITDTMKFFLYVLESGS | |
| | sequence | | |
| | not shown) | | |
| | Second Half | SQQIYGVVYGNVTFHVPSNVPLKECLWKKQKD | 1335 |
| | Antibody | KVAELENSEFRAFSSFKNRVYLDTVSCSLTIYNL | |
| | (includes Fc | TSSDEDEYEMESPNITDTMKFFLYVLESGSDKT | |
| | sequence) | HTCPPCPAPELLGGPSVFLFPPKPKDTLMISRTP | |
| | | EVTCVVVDVSHEDPEVKFNWYVDGVEVHNAKT | |
| | | KPREEQYASTYRVVSVLTVLHQDWLNGKEYKC | |
| | | KVSNKALPAPIEKTISKAKGQPREPQVYTLPPCR | |
| | | EEMTKNQVSLWCLVKGFYPSDIAVEWESNGQP | |
| | | ENNYKTTPPVLDSDGSFFLYSKLTVDKSRWQQ | |
| | | GNVFSCSVMHEALHNRYTQKSLSLSPGK | |
| | First Half | EIVMTQSPATLSLSPGERATLSCRASQDISKYLN | 1331 |
| | Antibody | WYQQKPGQAPRLLIYHTSRLHSGIPARFSGSGS | |
| | Light Chain | GTDYTLTISSLQPEDFAVYFCQQGNTLPYTFGQ | |
| | | GTKLEIKRTVAAPSVFIFPPSDEQLKSGTASVVC | |
| | | LLNNFYPREAKVQWKVDNALQSGNSQESVTEQ | |
| | | DSKDSTYSLSSTLTLSKADYEKHKVYACEVTHQ | |
| | | GLSSPVTKSFNRGEC | |
| CD19_CTL119 | First Half | QVQLQESGPGLVKPSETLSLTCTVSGVSLPDYG | 1328 |
| _CD3_16nM- | Antibody | VSWIRQPPGKGLEWIGVIWGSETTYYQSSLKSR | |
| CD58_IgV | Heavy Chain | VTISKDNSKNQVSLKLSSVTAADTAVYYCAKHY | |
| V45C_M114C | (Fc | YYGGSYAMDYWGQGTLVTVSSASTKGPSVFPL | |
| Trispecific | sequence | APSSKSTSGGTAALGCLVKDYFPEPVTVSWNS | |
| | not shown) | GALTSGVHTFPAVLQSSGLYSLSSVVTVPSSSL | |
| | | GTQTYICNVNHKPSNTKVDKRVEPKSCGGGGS | |
| | | GGGGSEVQLVESGGGLVQPGGSLKLSCAASGF | |
| | | TFNTYAMNWVRQASGKGLEWVGRIRSKYNNYA | |
| | | TYYADSVKDRFTISRDDSKSTLYLQMNSLKTED | |
| | | TAVYYCVRHGNFGNSYVSWFAYWGQGTLVTVS | |
| | | SGGGGSGGGGSGGGGSGGGGSQAVVTQEPS | |
| | | LTVSPGGTVTLTCRSSTGAVTTSNYANWVQQK | |
| | | PGQAPRGLIGGTNKRAPWTPARFSGSLLGDKA | |
| | | ALTLSGAQPEDEAEYFCALWYSNLWVFGGGTK | |
| | | LTVLGGGGS | |
| | First Half | QVQLQESGPGLVKPSETLSLTCTVSGVSLPDYG | 1094 |
| | Antibody | VSWIRQPPGKGLEWIGVIWGSETTYYQSSLKSR | |
| | Heavy Chain | VTISKDNSKNQVSLKLSSVTAADTAVYYCAKHY | |
| | (includes Fc | YYGGSYAMDYWGQGTLVTVSSASTKGPSVFPL | |
| | sequence) | APSSKSTSGGTAALGCLVKDYFPEPVTVSWNS | |
| | | GALTSGVHTFPAVLQSSGLYSLSSVVTVPSSSL | |
| | | GTQTYICNVNHKPSNTKVDKRVEPKSCGGGGS | |
| | | GGGGSEVQLVESGGGLVQPGGSLKLSCAASGF | |
| | | TFNTYAMNWVRQASGKGLEWVGRIRSKYNNYA | |
| | | TYYADSVKDRFTISRDDSKSTLYLQMNSLKTED | |
| | | TAVYYCVRHGNFGNSYVSWFAYWGQGTLVTVS | |
| | | SGGGGSGGGGSGGGGSGGGGSQAVVTQEPS | |
| | | LTVSPGGTVTLTCRSSTGAVTTSNYANWVQQK | |
| | | PGQAPRGLIGGTNKRAPWTPARFSGSLLGDKA | |
| | | ALTLSGAQPEDEAEYFCALWYSNLWVFGGGTK | |
| | | LTVLGGGGSDKTHTCPPCPAPELLGGPSVFLFP | |
| | | PKPKDTLMISRTPEVTCVVVAVSHEDPEVKFNW | |
| | | YVDGVEVHNAKTKPREEQYGSTYRVVSVLTVLH | |
| | | QDWLNGKEYKCKVSNKALAAPIEKTISKAKGQP | |
| | | REPQVCTLPPSRDELTKNQVSLSCAVKGFYPSD | |
| | | IAVEWESNGQPENNYKTTPPVLDSDGSFFLVSK | |
| | | LTVDKSRWQQGNVFSCSVMHEALHNHYTQKSL | |
| | | SLSPGK | |
| | Second Half | SQQIYGVVYGNVTFHCPSNVPLKEVLWKKQKD | 1336 |
| | Antibody | KVAELENSEFRAFSSFKNRVYLDTVSGSLTIYNL | |
| | (Fc | TSSDEDEYEMESPNITDTCKFFLYVLESGS | |
| | sequence | | |
| | not shown) | | |
| | Second Half | SQQIYGVVYGNVTFHCPSNVPLKEVLWKKQKD | 1337 |
| | Antibody | KVAELENSEFRAFSSFKNRVYLDTVSGSLTIYNL | |
| | (includes Fc | TSSDEDEYEMESPNITDTCKFFLYVLESGSDKT | |
| | sequence) | HTCPPCPAPELLGGPSVFLFPPKPKDTLMISRTP | |
| | | EVTCVVVDVSHEDPEVKFNWYVDGVEVHNAKT | |
| | | KPREEQYASTYRVVSVLTVLHQDWLNGKEYKC | |
| | | KVSNKALPAPIEKTISKAKGQPREPQVYTLPPCR | |
| | | EEMTKNQVSLWCLVKGFYPSDIAVEWESNGQP | |
| | | ENNYKTTPPVLDSDGSFFLYSKLTVDKSRWQQ | |
| | | GNVFSCSVMHEALHNRYTQKSLSLSPGK | |
| | First Half | EIVMTQSPATLSLSPGERATLSCRASQDISKYLN | 1331 |
| | Antibody | WYQQKPGQAPRLLIYHTSRLHSGIPARFSGSGS | |
| | Light Chain | GTDYTLTISSLQPEDFAVYFCQQGNTLPYTFGQ | |
| | | GTKLEIKRTVAAPSVFIFPPSDEQLKSGTASVVC | |
| | | LLNNFYPREAKVQWKVDNALQSGNSQESVTEQ | |
| | | DSKDSTYSLSSTLTLSKADYEKHKVYACEVTHQ | |
| | | GLSSPVTKSFNRGEC | |

Trispecific binding molecules were expressed transiently by co-transfection of the respective chains in HEK293 cells.

Briefly, transfection was performed using PEI as transfection reagent. For small scale (<5L) transfections, cells were grown in shake flasks on an orbital shaker (115 rpm) in a humidified incubator (85%) at 5% C02). Plasmids were combined with PEI at a final ratio of 1 DNA:3 PEI. 1 mg/L culture of plasmid was used for transfection at 2.0 million cells/mL serum media. After 5 days of expression, the TBMs were harvested by clarification of the media via centrifugation and filtration. Purification was performed via anti-CH1 affinity batch binding (CaptureSelect IgG-CH1 Affinity Matrix, Thermo-Fisher Scientific, Waltham, MA, USA) or Protein A (rProteinA Sepharose, Fast flow, GE Healthcare, Uppsala, Sweden) batch binding using 1 ml resin/100 mL supernatant. The protein was allowed to bind for a minimum of 2 hours with gentle mixing, and the supernatant loaded onto a gravity filtration column. The resin was washed with 20-50 CV of PBS. TBMs were eluted with 20 CV of 50 mM citrate, 90 mM NaCl pH 3.2. 50 mM sucrose The eluted TBMs were adjusted to pH 5.5 with 1 M sodium citrate 50 mM sucrose. Preparative size exclusion chromatography was performed using Hi Load 16/60 Superdex 200 grade column (GE Healthcare Life Sciences, Uppsala, Sweden) as a final polishing step when aggregates were presente. To confirm that the identity of the proteins of the TBMs expressed matched the predicted masses for the primary amino acid sequences, proteins were analyzed by high-performance liquid chromatography coupled to mass spectrometry.

### 8.13.2. Results

As shown in Table 25 below, inclusion of stabilizing disulfide variants had no adverse impact on overall expression yields of increased aggregate content upon purification.

TABLE 25

| | Expression | |
|---|---|---|
| | (mg/L) | % HMWS |
| CD19_CTL119_CD3_16nM-CD58_Full | 20 | <10% |
| ECD Trispecific | | |
| (Full ECD WT) | | |
| CD19_CTL119_CD3_16nM-CD58_IgV | 20 | ~10 |
| Trispecific | | |
| (IgV WT) | | |
| CD19_CTL119_CD3_16nM- | 55 | ~10 |
| CD58_IgV_V45C_M105C Trispecific | | |
| (IgV V45C_M105C) | | |
| CD19_CTL119_CD3_16nM-CD58_IgV | 65 | ~10 |
| V54C_G88C Trispecific | | |
| (IgV V54C_G88C) | | |
| CD19_CTL119_CD3_16nM-CD58_IgV | 63 | ~10 |
| V45C_M114C Trispecific | | |
| (IgV V45C_M114C) | | |

### 8.14. Example 14: Re-Directed T Cell Cytotoxicity with TBMs Containing CD58 Variants

TBMs of Example 13 containing the variant CD58 domains were analyzed for their potential to induce T cell-mediated apoptosis in tumor target cells.

### 8.14.1. Materials and Methods

Briefly, huCD19-expressing Nalm6 target cells were engineered to overexpress firefly luciferase. Cells were harvested and resuspendend in RPMI medium (Invitrogen #11875-093) with 10% FBS. 10,000 target cells per well were plated in a flat-bottom 96-well plate. Human pan T effector cells were isolated via MACS negative selection (Miltenyi Biotec #130-096-535) from two donors from cryopreserved PBMC (Cellular Technologies Limited #CTL-UP1) then added to the plate to obtain a final E:T ratio of 5:1. Co-cultured cells were incubated with a serial dilution of all constructs and controls. For normalization, average maximum luminescence refers to target cells co-incubated with effector cells, but without any test construct. After an incubation of either 24 or 48 hr at 37° C., 5% C02, OneGlo luciferase substrate (Promega #E6120) was added to the plate. Luminescence was measured on an Envision plate reader after a 10 minute incubation. Percent specific lysis was calculated using the following equation: Specific lysis (%)=(1−(sample luminescence/average maximum luminescence))*100

### 8.14.2. Results

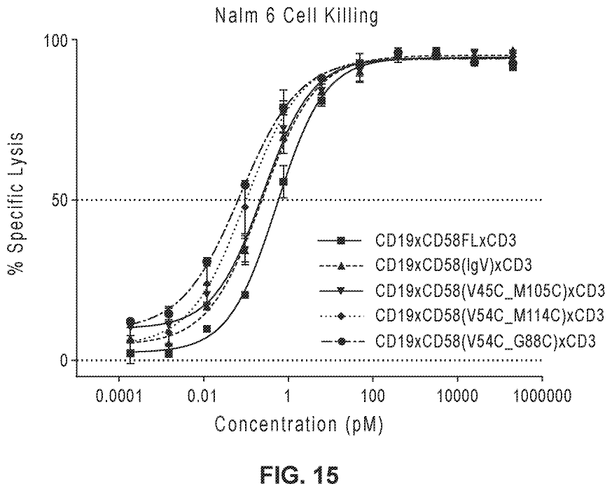

As shown in FIG. 15, the TBMs containing the variant CD58 domains show comparable cytotoxic activity to a TBM with wild type CD58.

### 8.15. Example 15: T-Cell Activation with TBMs Containing CD58 Variants

As an alternative to primary T cell activation, a Jurkat-NFAT reporter cell line was used to evaluate the functional activity of the TBMs of Example 13 containing the variant CD58 domains.

### 8.15.1. Materials and Methods

The Jurkat T cell line (E6-1) was transfected with a NFAT-luciferase reporter construct and a stable, clonal cell line Jurkat cells with NFAT-LUC reporter (JNL), was selected for further characterization based on strong induction of the NFAT reporter following PMA and ionomycin stimulation.

The Jurkat reporter cell line for was used for determination of non-specific activation of NFAT.

Purified TBMs were tested for their potential to induce NFAT activation in the absence of target cells.

Jurkat cells with NFAT-LUC reporter (JNL) were grown in RPMI-1640 media containing 2 mM glutamine and 10% fetal bovine serum with puromycin at 0.5 ug/ml. 100,000 JNL cells per well were plated in a flat-bottom 96-well plate and were incubated with serial dilutions of the TBMs and controls. After an incubation of 6 hr at 37° C., 5% C02, OneGlo luciferase substrate (Promega #E6120) was added to the plate. Luminescence was measured on an Envision plate reader after a 10 minute incubation.

### 8.15.2. Results

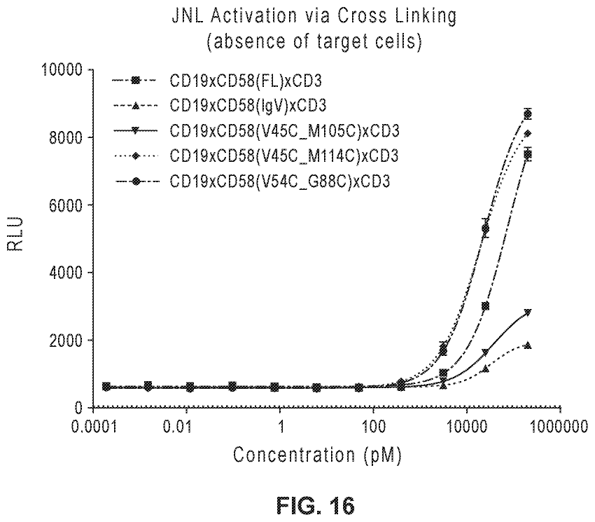

As shown in FIG. 16, the TBMs containing the variant CD58 domains show tumor-independent (i.e., non-target cell specific) activitation levels comparable to or lower than TBMs containing wild type CD58.

### 8.16. Example 16: CD19 and CD58 Expression on Various Cell Lines

### 8.16.1. Materials and Methods

Cell surface expression of CD19 and CD58 was determined on OCI-LY-19 (a human B-cell non-Hodgkin lymphoma cell line), Karpas-422 (a human B-cell non-Hodgkin lymphoma cell line), Toledo (a human B-cell non-Hodgkin lymphoma cell ine), and Nalm-6 (B cell precursor leukemia cell line) cell lines by flow cytometry using APC labelled anti-CD19 (Biolegend, Cat #302212) and APC-labelled anti-CD58 (Biolegend, Cat #330918) and respective isotype control antibodies. The samples were run on BD LSR Fortessa and analyzed using FlowJo.

### 8.16.2. Results

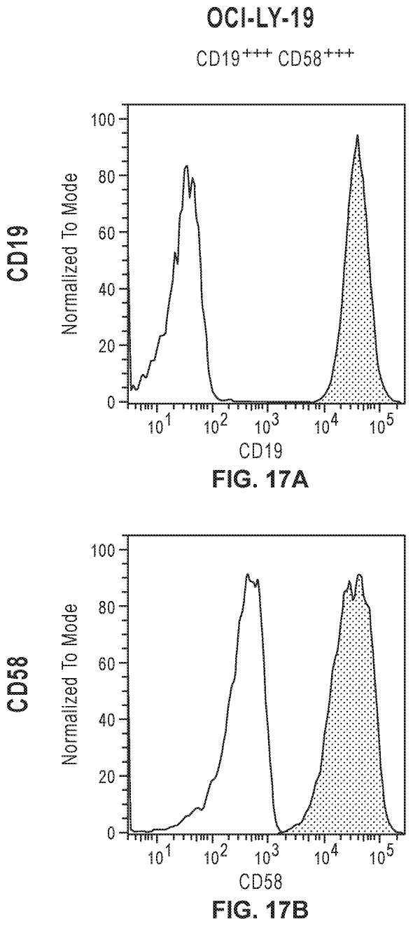

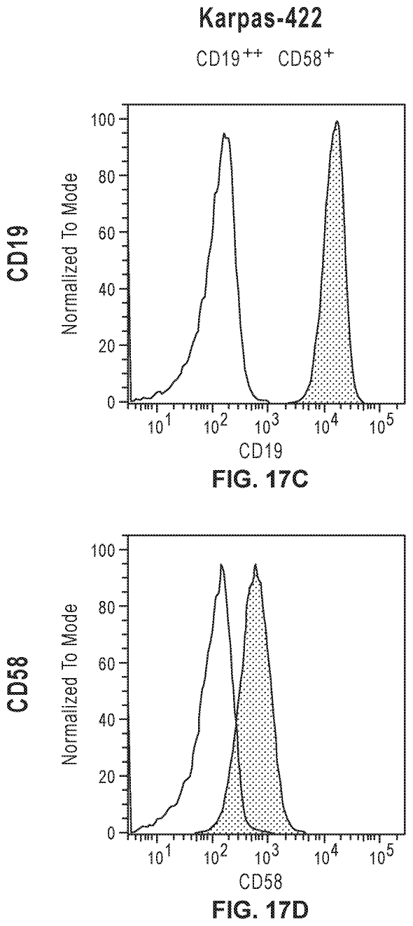

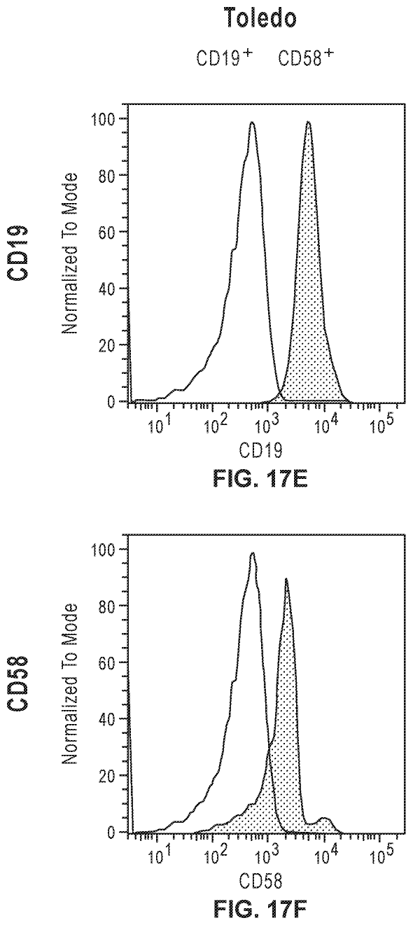

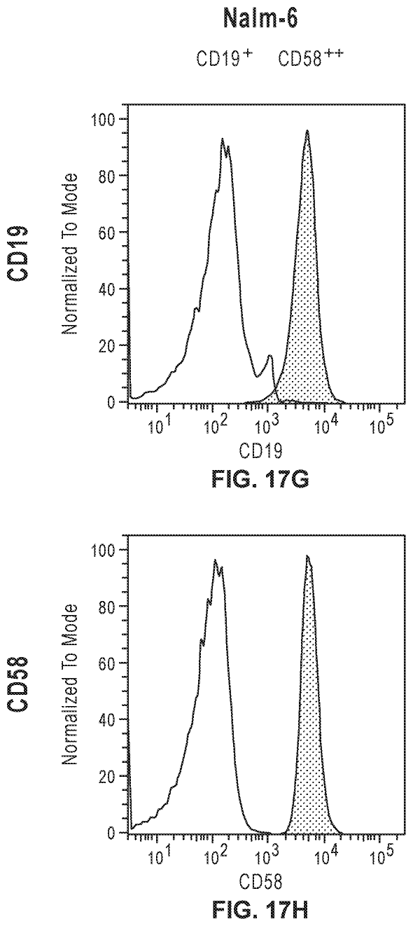

The cell lines have different level of CD19 and CD58 expression (FIGS. 17A-H). The ranking for CD19 expression among the cell lines was OCI-LY-19>Karpas 422>Toledo=Nalm-6. The ranking for CD58 expression was OCI-LY-19>Nalm-6>Karpas=Toledo.

### 8.17. Example 17: RTCC and Cytokine Secretion Activity of the NEG258-Based TBMs Vs. A One-Arm BBM that does not Bind to CD2 and a TBM that does not Bind to CD19

CD3hi TSP1, CD3med TSP1, CD3 hi BSP1, and CD3hi TSP1C (H variants) were compared for their potential to induce T cell-mediated apoptosis in Karpas422 target cells.

### 8.17.1. Materials and Methods

An RTCC assay with huCD19-expressing Karpas422 target cells was performed according to the Materials and Methods described in Example 9, but with a final E:T ratio of 1:1 and a 96 hour incubation.

### 8.17.2. Results

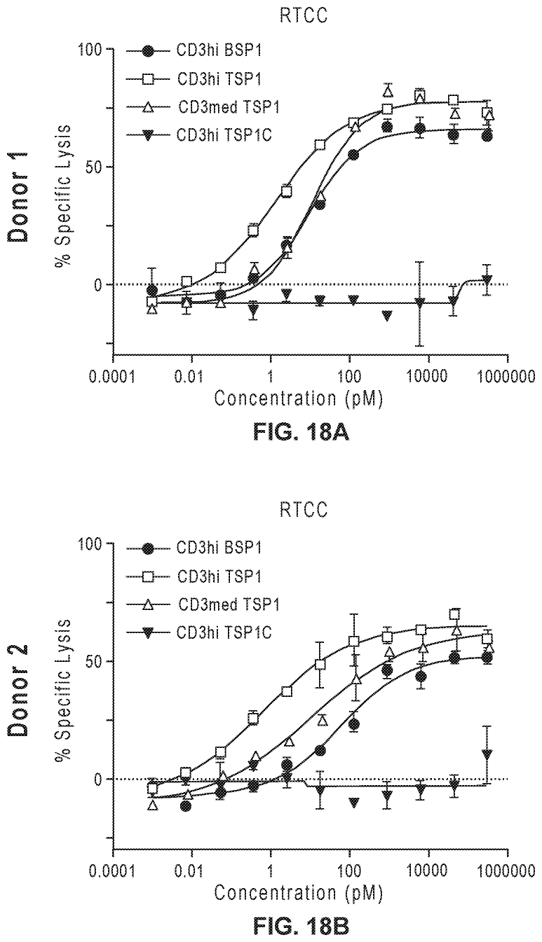

As shown in FIGS. 18A-18B, CD3hi TSP1, CD3med TSP1, and CD3hi BSP1 show cytotoxic activity against Karpas422 target cells, with CD3hi TSP1 having the highest cytotoxic activity.

### 8.18. Example 18: Cytokine Release Assay

CD3hi TSP1, CD3med TSP1, CD3 hi BSP1, and CD3hi TSP1C (H variants) were analyzed for their ability to induce T cell-mediated de novo secretion of cytokines in the presence of Karpas422 cells.

### 8.18.1. Materials and Methods

A cytokine release assay was performed as in Example 10, but with Karpas422 cells at a final E:T ratio of 1:1 and an incubation of 48 hours.

### 8.18.2. Results

As shown in FIGS. 19A-19F, CD3hi TSP1, CD3med TSP1, and CD3hi BSP1 induced cytokine secretion by T cells, with CD3hi TSP1 inducing the highest levels of cytokine secretion, followed by CD3med TSP1, which was similar to CD3hi BSP1.

### 8.19. Example 19: TBM and BBM Binding to T Cells

Binding of CD3hi TSP1, CD3med TSP1, CD3hi BSP1, and CD3hi TSP1C (H variants) to T cells was evaluated using flow cytometry.

### 8.19.1. Materials and Methods

Peripheral blood mononuclear cells (PBMCs) previously isolated and cryopreserved from 2 Leukopak donors (Hemacare) were thawed and Pan T cells were isolated by negative selection using the Pan T cell Isolation Kit, human (Miltenyi Biotec, Cat #130-096-535) following the manufacturer's protocol. T cells were resuspended in FACS Buffer and 100,000 cells were added to each well of a 96 well round bottom plate. A dilution series of CD3med TSP1, CD3hi TSP1, CD3hi BSP1, and CD3hi TSP1C ranging from 33 μg/ml-0.005 μg/ml was added to cells and incubated on ice for 1 hour. Cells were washed twice, resuspended in 100 μl of anti-human IgG secondary antibody and incubated on ice for another hour. After the incubation, cells were washed twice, resuspended in 100 μl of fixable viability dye and incubated on ice for 30 min. After washing twice again, cells were resuspended in 120 μl of FACS buffer. The cells were then run on BD LSR Fortessa and data was analyzed using FlowJo to determine the MFI of anti-human IgG secondary antibody, which was plotted against antibody concentration.

### 8.19.2. Results

All antibodies showed different degree of binding to T cells (FIG. 20). CD3hi TSP1 was the strongest binder followed by CD3med TSP1, with BSP1 being the weakest binder. Without being bound by theory, it is believed that the improved binding of the TBMs can be attributed to co-engagement of CD2 and CD3 arms, thereby increasing the binding avidity to T cells.

### 8.20. Example 20: TBM and BBM Mediated T Cell Proliferation

CD3hi TSP1, CD3med TSP1, CD3hi BSP1, and CD3hi TSP1C (H variants), and blinatumomab were evaluated for their ability to induce T cells proliferation upon co-culture with CD19 expressing OCI-LY-19, Karpas422, and Toledo target cells.

### 8.20.1. Materials and Methods

Briefly, OCI-LY-19, Karpas422, and Toledo target cells stably expressing firefly luciferase were plated in a 96 well plate in T Cell Media (TCM) (RPMI-1640, ThermoFisher Scientific, Cat #11875-085), 10% FBS (Seradigm, Cat #1500-500), 1% L-Glutamine (Thermo Fisher Scientific, Cat #25830-081), 1% Non Essential Amino Acids (Thermo Fisher Scientific, Cat #11140-050), 1% Pen/Strep (Thermo Fisher Scientific, Cat #15070063), 1% HEPES (Thermo Fisher Scientific, Cat #15630080), Sodium Pyruvate (Thermo Fisher Scientific, Cat #11360-070), 0.1% Beta-mercaptoethanol (Thermo Fisher Scientific, Cat #21985-023)]. PBMCs previously isolated and cryopreserved from 2 Leukopak donors were thawed and Pan T cells were isolated (as described earlier). Isolated T cells were labelled with 5 μM Cell Trace Violet (CTV) (Thermo Fisher Scientific, Cat #C34557) following the manufacturer's protocol and were co-cultured with target cells at an E:T ratio of 1:3. A dilution series of CD3med TSP1, CD3hi TSP1, CD3hi BSP1, CD3hi TSP1C, and blinatumomab ranging from 2.5 nM-0.0006 nM was added to cells and the plates were incubated in a 5% C02, 37° C. incubator for 96 hrs. After incubation, the cells were harvested, treated with Human TruStain FcX (Fc Block) (Biolegend, Cat #422302) and stained with Fixable Viability Dye eFlour 780 (ThermoFisher Scientific, Cat #65-0865-14), followed by staining with PerCP-Cy5.5 conjugated anti-human CD3 mAb (Biolegend, Cat #317336). All staining steps were performed according to manufacturer's protocol. Flow analysis were performed using BD LSR Fortessa and FlowJo software to determine % proliferated CD3+ T cells based on CD3 staining and dilution of Cell Trace Violet dye.

### 8.20.2. Results

All CD19 targeting antibodies induced proliferation of T cells upon co-culture with different CD19 expressing target cell lines (FIGS. 21A-21C). The T cell proliferation effect was dose-dependent, and CD3hi TSP1 showed more potent activity than CD3med TSP1 and CD3hi BSP1. The control antibody did not induce any T cell proliferation indicating that CD19 target-specific engagement was required for the proliferation of T cells. Blinatumomab mediated the most potent T cell proliferation in the presence of OCI-LY-19 and Toledo cells. In the presence of Karpas420, CD3hi TSP1 more effectively induced T cell proliferation as shown by the maximum percentage of proliferated T cells.

### 8.21. Example 21: RTCC Activity of NEG258-Based TBMs with Different CD3 Affinities Vs. A BBM and Blinatumomab

The NEG258-based TBMs containing CD3 binding arms with different affinities (CD3hi TSP1 and CD3med TSP1 (H variants)) and a BBM (CD3hi BSP1 (H variant)) were compared for their potential to induce T cell-mediated apoptosis in Karpas422 target cells. The study also included blinatumomab as a control.

### 8.21.1. Materials and Methods

An RTCC assay with huCD19-expressing Karpas422 target cells was performed according to the Materials and Methods described in Example 9, but with a final E:T ratio of 1:1 and a 96 hour incubation.

### 8.21.2. Results

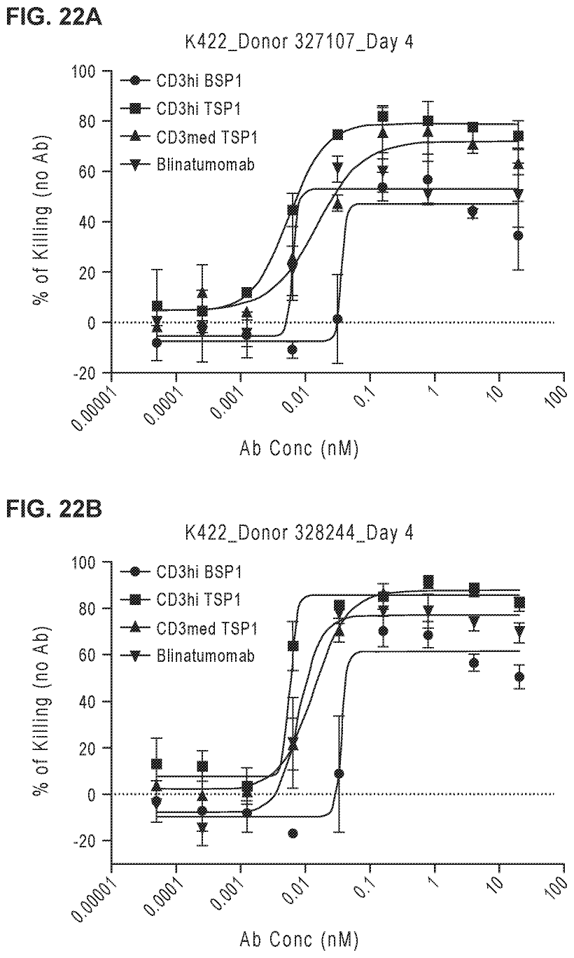

As shown in FIGS. 22A-22B, both types of TBMs show cytotoxic activity against Karpas422 cells. The TBMs demonstrated superior cytotoxic activity compared to the BBM. CD3hi TSP1 showed similar or superior cytotoxic activity compared to blinatumomab.

### 8.22. Example 22: RTCC Activity of NEG258-Based TBMs with Different CD3 Affinities Vs. A BBM and TBMs that do not Bind to CD19 Against Multiple B Cell Lymphoma Cell Lines

CD3hi TSP1, CD3med TSP1, CD3hi BSP1, and CD3hi TSP1C (H variants) were compared for their potential to induce T cell-mediated apoptosis in Oci-Ly19, Toledo, Nalm6, Nalm6 KO and K562 target cells. Oci-Ly19, Toledo, Nalm6 cells express hCD19 antigen. Nalm6 KO and K562 target cells lacking hCD19 expression were used to assess target-independent killing. The study also included blinatumomab as a control.

### 8.22.1. Materials and Methods

Nalm6 KO was generated from Nalm6 parental cell line by using CRISPR-CAS9 technology and was confirmed to lack hCD19 expression. Oci-Ly19, Toledo, Nalm6, Nalm6 KO and K562 target cells were engineered to overexpress firefly luciferase. RTCC assays were performed with the different cells lines according to the Materials and Methods described in Example 9, but with a final E:T ratio of 1:1 and a 48 hour incubation.

### 8.22.2. Results

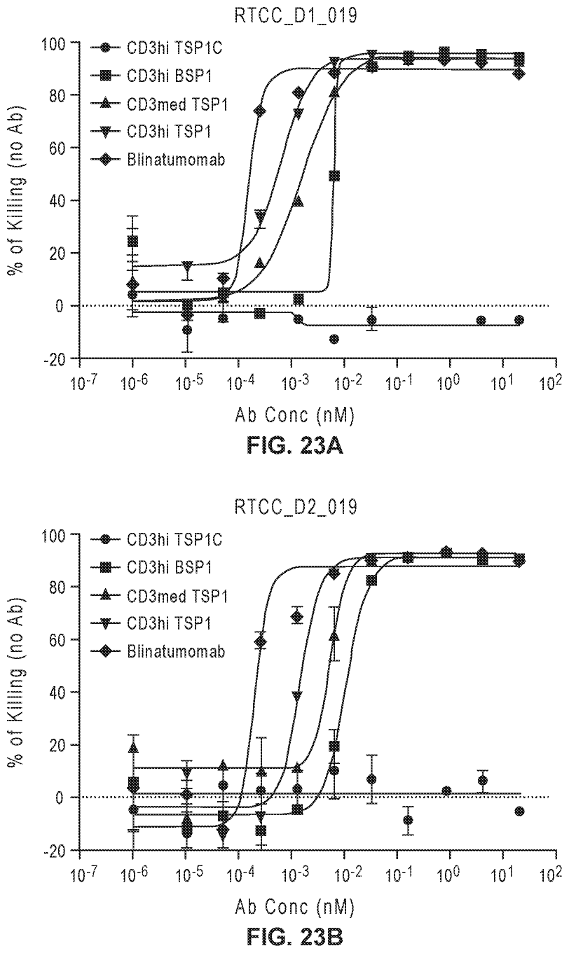

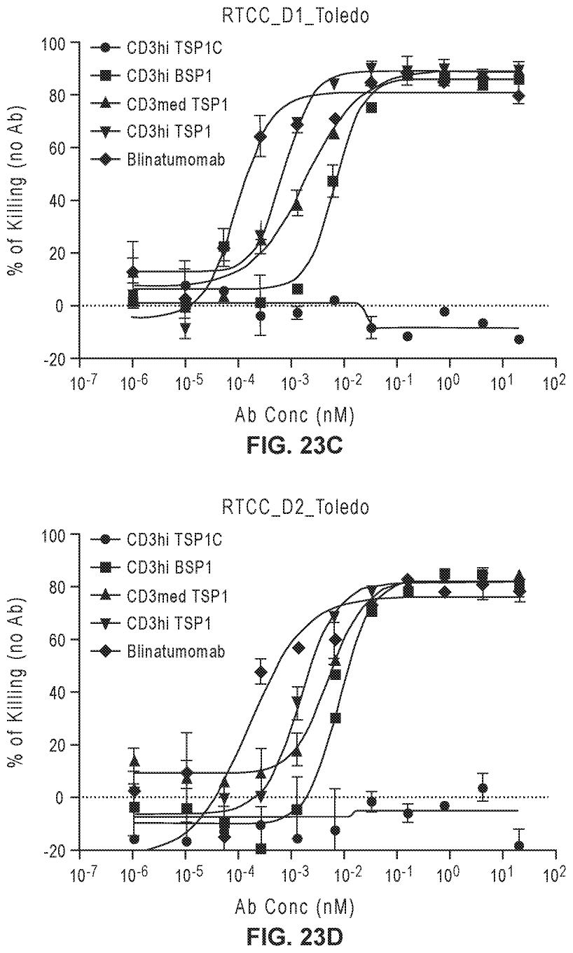

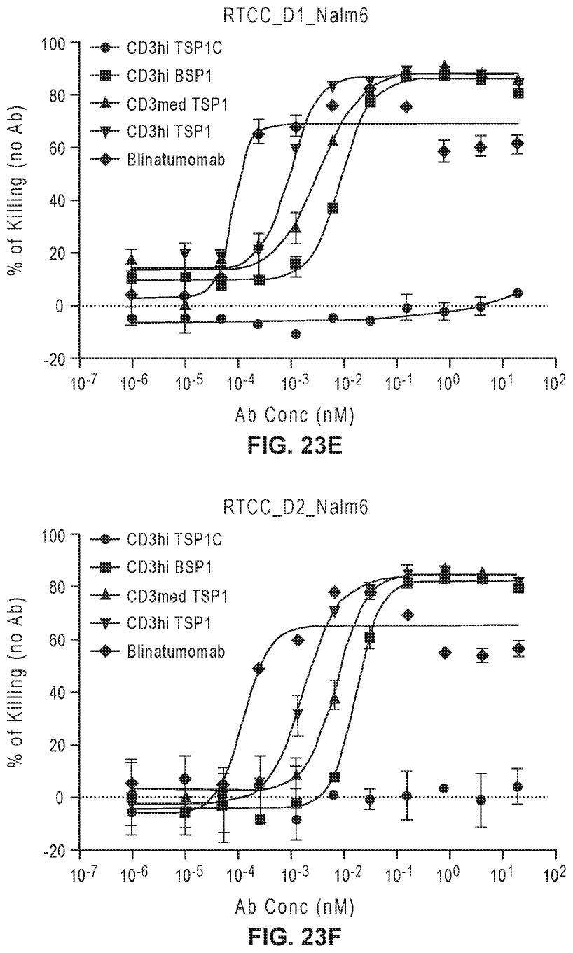

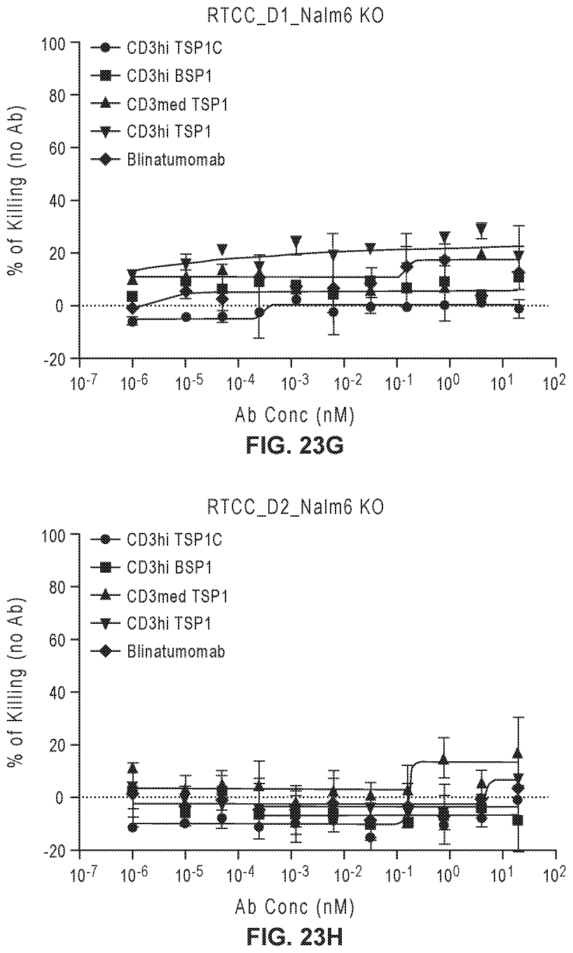

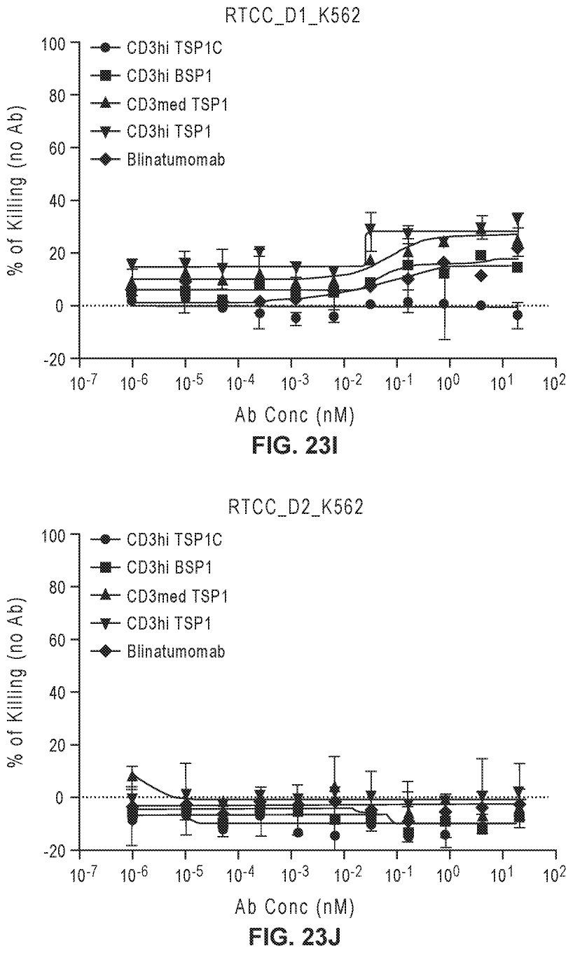

CD3hi TSP1 and CD3med TSP1 showed cytotoxic activity against Oci-Ly19, Toledo and Nalm6, but showed minimal activity against antigen-negative Nalm6 KO and K562 (FIGS. 23A-23J). The TBMs demonstrated superior cytotoxic activity compared to the BBM. CD3hi TSP1 showed comparable cytotoxic activity to blinatumomab.

### 8.23. Example 23: Cytokine Release Assay of the NEG258-Based TBMs with Different CD3 Affinities Vs. A BBM and TBMs that do not Bind to CD19 Against Multiple B Cell Lymphoma Cell Lines

CD3hi TSP1, CD3med TSP1, CD3hi BSP1 and CD3hi TSP1C (H variants) were compared for their potential to induce T cell-mediated de novo secretion of cytokines in Oci-Ly19, Toledo, Nalm6, Nalm6 KO and K562 target cells. Oci-Ly19, Toledo, Nalm6 cells express hCD19 antigen. Nalm6 KO and K562 target cells that lack hCD19 expression were used to assess target-independent cytokine release. The study also included blinatumomab as a control.

### 8.23.1. Materials and Methods

Target cells were harvested and resuspended in RPMI medium (Invitrogen #11875-093) with 10% FBS. 5,000 target cells per well were plated in a flat-bottom 384-well plate. Human pan T effector cells were isolated via negative selection (Stemcell Technologies #17951) from two donors from cryopreserved PBMCs that were separated from a leukopak (Hemacare #PB001F-1) by Ficoll density gradient centrifugation. Purified T cells were then added to the plate to obtain a final E:T ratio of 1:1. After an incubation of 48 hr at 37° C., 5% C02, the supernatants were harvested for subsequent analysis. A multiplexed ELISA was performed according to the manufacturer's instructions using a human cytokine custom 3-plex 384 4-spot kit (MesoScale Discovery #N31IB-1).

### 8.23.2. Results

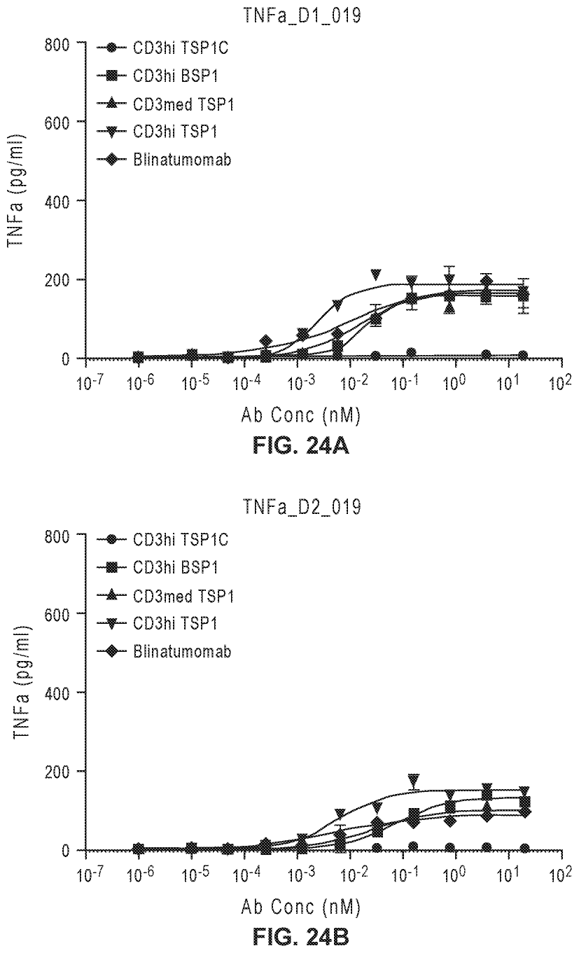

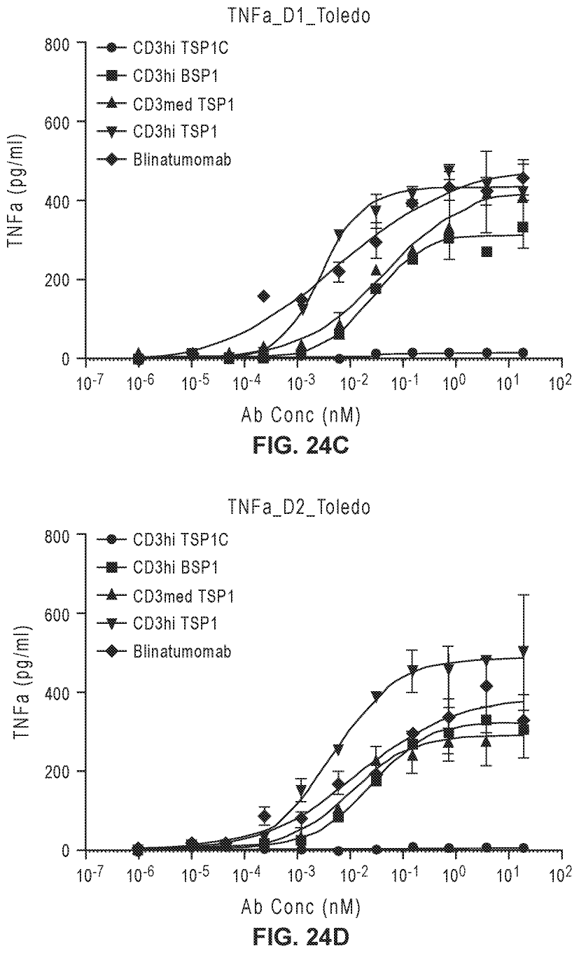

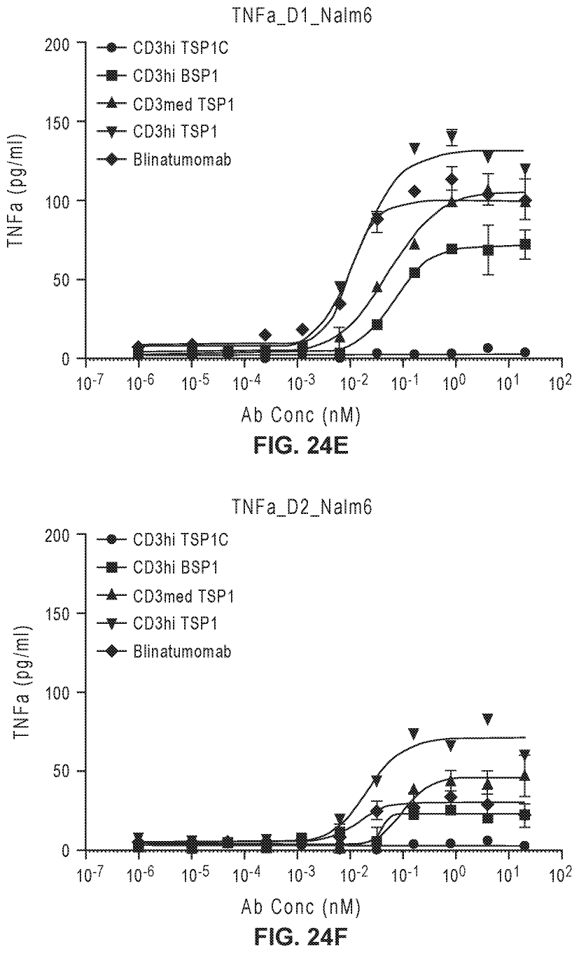

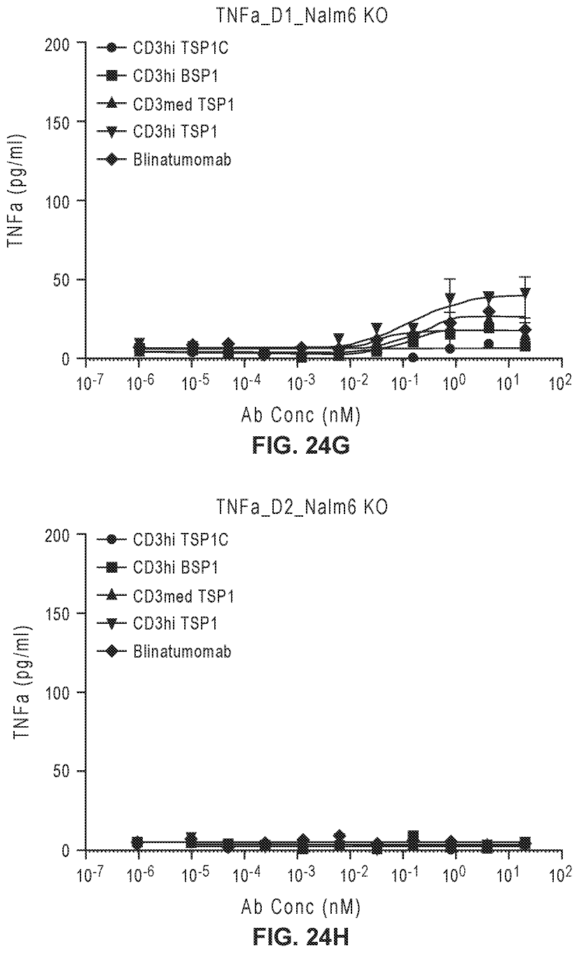

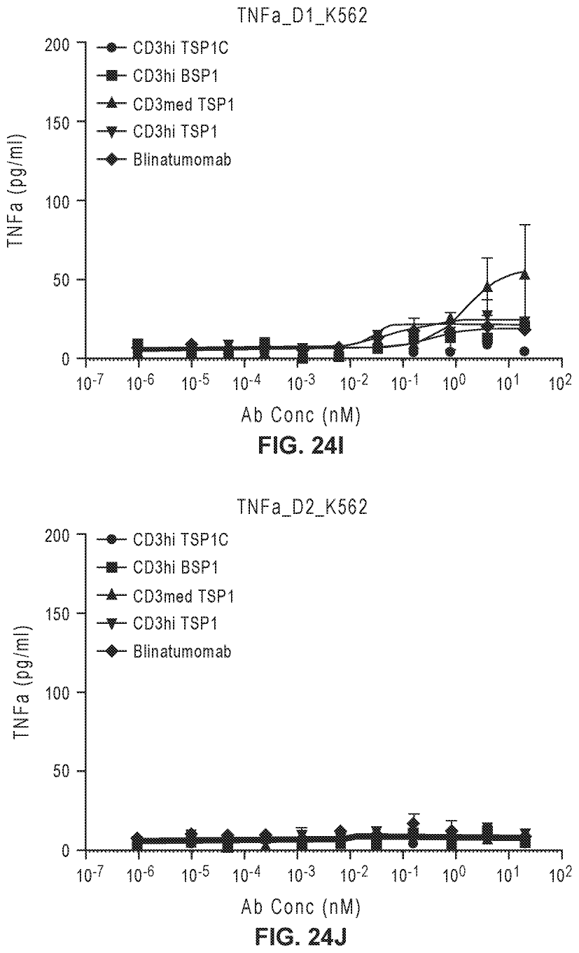

As shown in FIGS. 24A-24J, both NEG258-based TBMs induce significant cytokine secretion by T cells in a dose-dependent manner when incubated with Oci-Ly19, Toledo and Nalm6 cells. Minimal cytokine secretion was detected when incubating with antigen-negative Nalm6 KO and K562.

### 8.24. Example 24: Re-Challenge RTCC Assay with Karpas 422 & OCI-LY-19 Cell Lines

The effect of target cell re-challenge on the killing activity of CD3hi TSP1 (H variant), CD3med TSP1 (H variant), CD3hi BSP1 (H variant), and blinatumomab treated T cells was determined using a dose titration re-challenge RTCC assay.

### 8.24.1. Materials and Methods

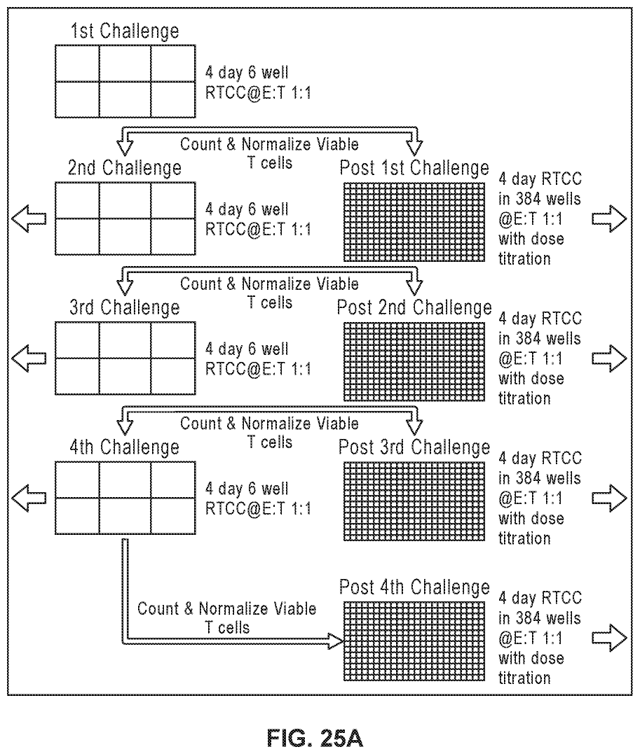

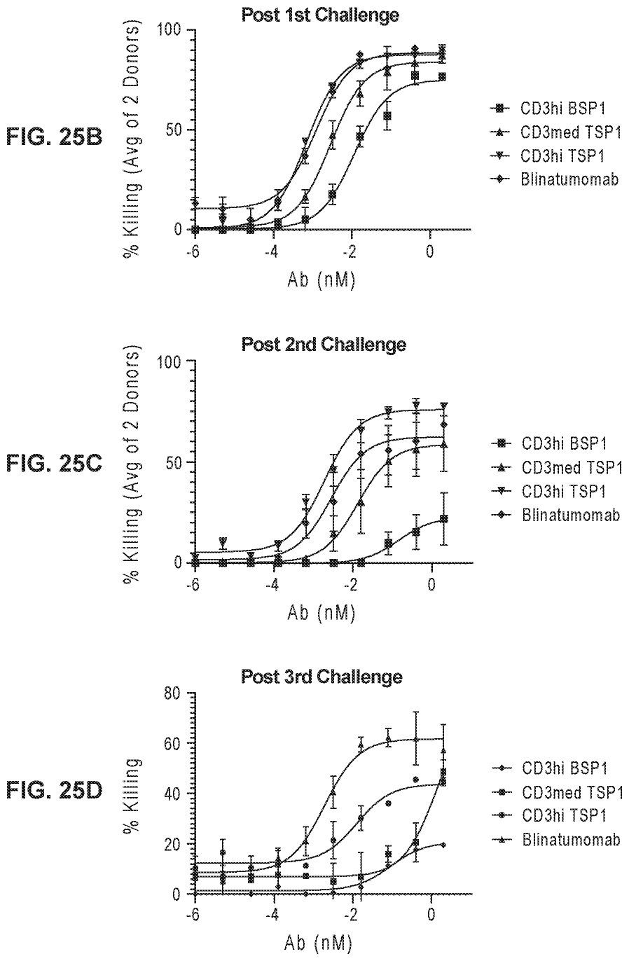

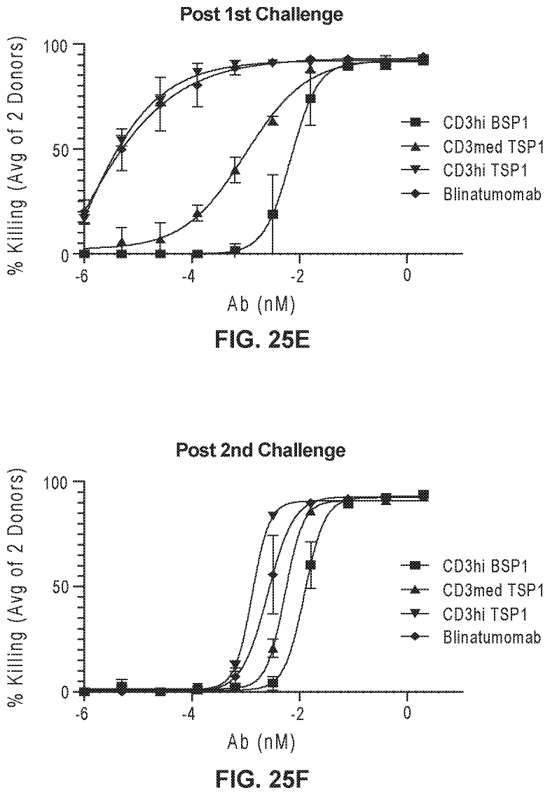

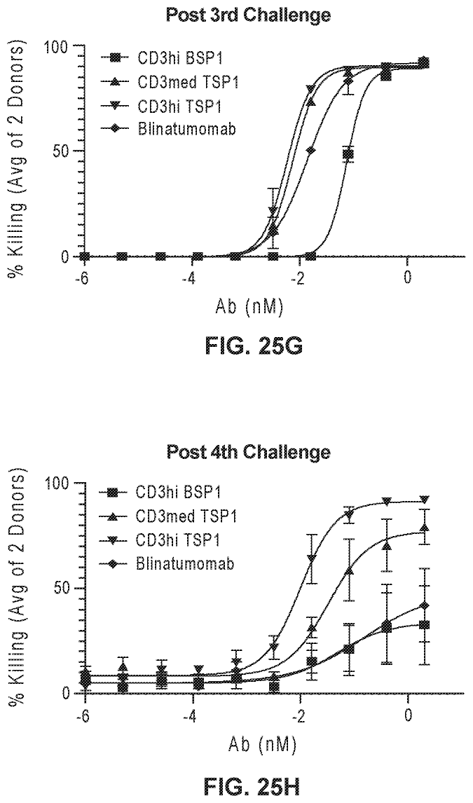

OCI-LY-19 and Karpas422 target cells stably expressing firefly luciferase were plated in a Costar 6 well plate in T Cell Media (TCM). PBMCs previously isolated and cryopreserved from 2 Leukopak donors were thawed and Pan T cells were isolated (as described earlier). A co-culture of T cells and OCI-LY-19 or Karpas 422 cells at E:T ratio of 1:1 along with EC90 concentration (0.1 nM for OCI-LY-19 and 0.5 nM for Karpas 422) of CD3med TSP1, CD3hi TSP1, CD3hi BSP1, and blinatumomab was set-up. The plates were incubated for 4 days for OCI-LY-19 and 5 days for Karpas 422 cells. At the end of incubation, the killing of target cells was determined using the luminescence signal. The absolute T cell counts from each antibody treated condition was also determined. For the next round of rechallenge, it was ensured that the killing of target cells was equivalent across various antibody conditions. The T cell counts were normalized across different antibody conditions and another round of a single concentration rechallenge was set-up at E:T of 1:1 using the EC90 concentration with a 4 day incubation for both target cells. Additionally, a dose titration RTCC at a E:T of 1:1 and a concentration range of 2 nM −0.000001 nM was set-up using T cells from the different antibody treated conditions with a 4 day incubation used for each cell line. The killing of target cells was determined using the luminescence signal to generate dose response curves. At the end of the challenge, the above process was repeated once more for Karpas 422 and twice more for OCI-LY-19 cells. The assay set-up is shown in FIG. 25A.

### 8.24.2. Results

As can be seen from FIGS. 25B-25H, CD3hi TSP1 was able to better retain killing ability upon repeated challenges with the target cells compared to CD3med TSP1, and CD3hi TSP1. CD3med TSP1 was the next best, with CD3hi BSP1 being the least active of all antibodies. CD3hi TSP1 demonstrated similar activity when compared to Blinatumomab in the first 2 or 3 rounds of re-challenges for Karpas422 and OCI-LY-19 cells, respectively. At the last re-challenge for OCI-LY-19, CD3hi TSP1 mediated more potent RTCC than blinatumomab (both in EC50 and maximum lysis), whereas for Karpas422, blinatumomab mediated higher maximum lysis than CD3hi TSP1 despite a similar EC50.

### 8.25. Example 25: Re-Challenge T Cell Phenotyping with Karpas 422 & OCI-LY-19 Cell Lines

The effect of target cell re-challenge on the phenotype of CD3hi TSP1, CD3med TSP1, and CD3hi BSP1 (H variants) treated T cells was determined using a single concentration re-challenge assay.

### 8.25.1. Materials and Methods

OCI-LY-19 and Karpas422 target cells stably expressing firefly luciferase were plated in a Costar 6 well plate in T Cell Media (TCM). PBMCs previously isolated and cryopreserved from 2 Leukopak donors were thawed and Pan T cells were isolated (as described earlier). Co-cultures of T cells and OCI-LY-19 or Karpas 422 cells at E:T ratio of 1:1 was set-up and 1 nM of CD3hi BSP1, CD3med TSP1, or CD3hi TSP1 was added. The plates were incubated for 4 days for OC-LY-19 and 5 days for Karpas 422 cells. At the end of incubation, the killing of target cells and absolute T cell counts from each antibody treated condition was determined. The T cell counts were normalized across different antibody conditions and two additional rounds of re-challenges were set-up the same way as the previous challenge with a 4 day incubation for both target cells, for a total of three challenges. After the third challenge, T cells from different antibody treated conditions were harvested on day 2 from the Karpas 422 co-cultures and on day 4 from OCI-LY-19 co-cultures and split into 2 fractions. One fraction was stained with blue fixable viability dye (ThermoFisher Scientific, Cat #L23105) prior to staining with a cocktail of anti-human CD3 (Biolegend, Cat #317324), CD4 (Biolegend, Cat #344608), CD8 (BD Biosciences, Cat #563795), CD27 (Biolegend, Cat #356412) & CD62L mAb (Biolegend, Cat #304814). The second fraction was resuspended to 1e6/ml in TCM and stimulated with Cell Stimulation Cocktail (Tonbo Biosciences, Cat #TNB4975) for 4 hrs at 37° C. Thereafter the cells were washed and sequentially stained with blue fixable viability dye (ThermoFisher Scientific, Cat #L23105), a cocktail of anti-human CD3 (Biolegend, Cat #317324), CD4 (Biolegend, Cat #344608), CD8 (BD Biosciences, Cat #563795), followed by permeabilization using the FoxP3 transcription factor staining set (ThermoFisher Scientific, Cat #00-5523-00) and final staining with anti-human IFNγ mAb (Biolegend, Cat #400134) and IL-2 mAb (Biolegend, Cat #400551) or respective isotype controls. All stainings were performed according to manufacturer's protocol. Flow analyses were performed using BD LSR Fortessa and FlowJo software.

### 8.25.2. Results

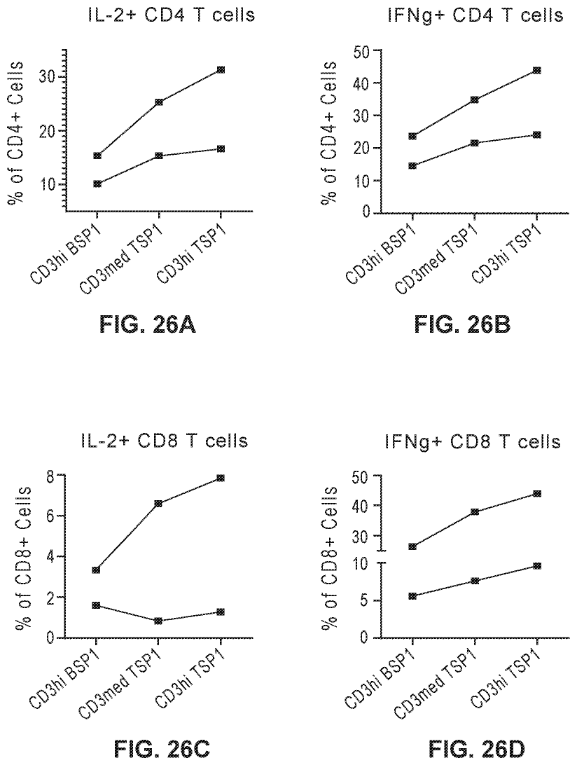

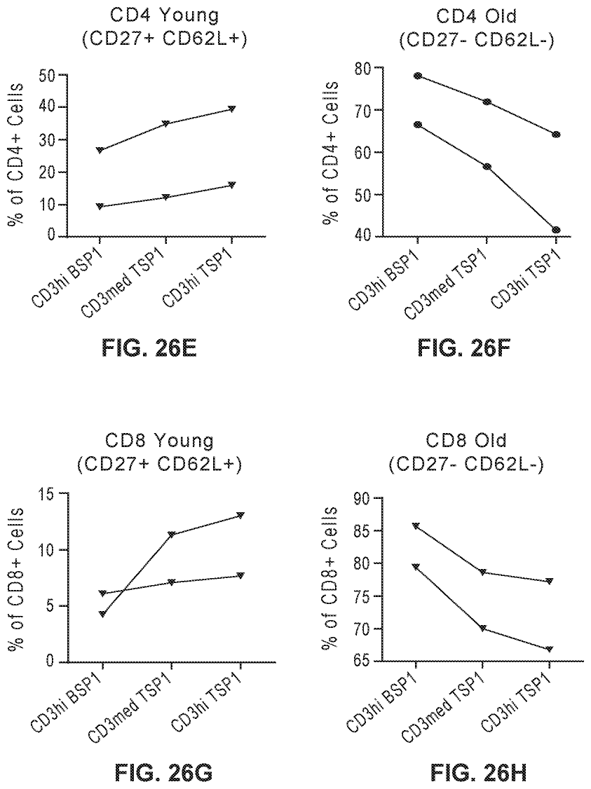

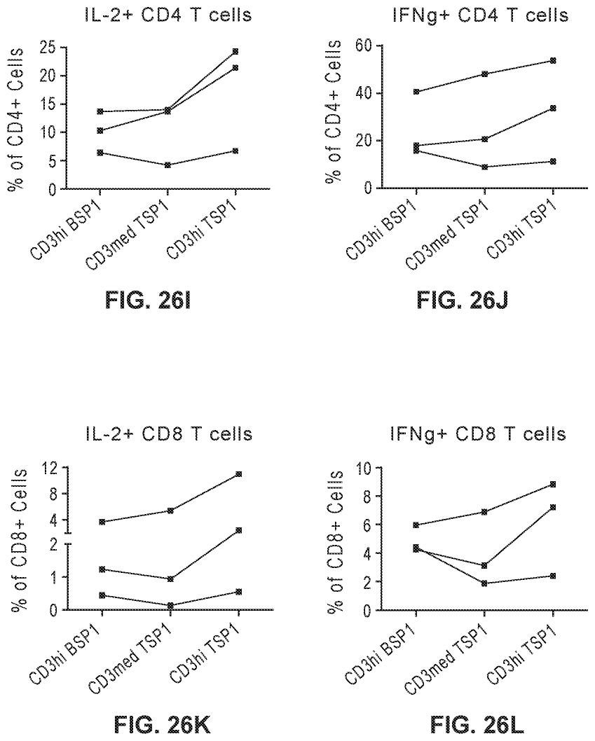

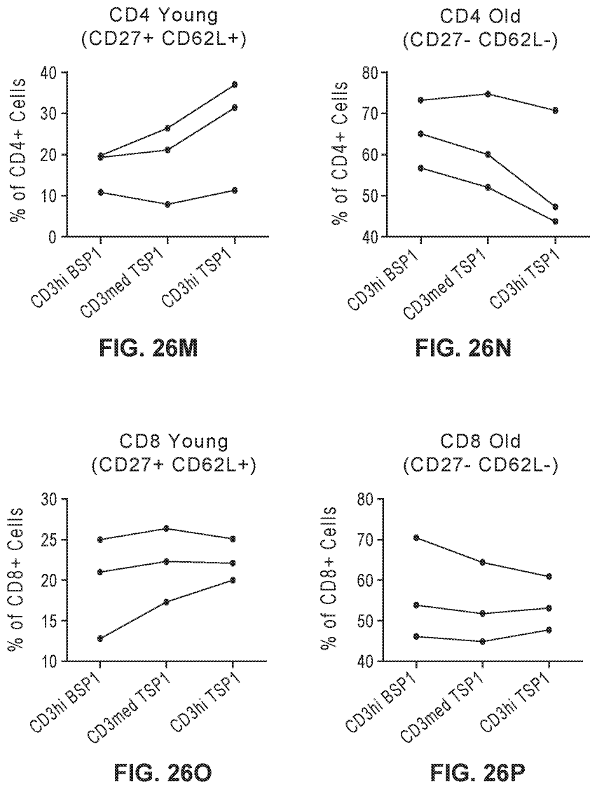

As shown in FIGS. 26A-26H (Karpas 422 model) and FIGS. 26I-26P (OCI-LY-19 model), CD3hi TSP1 better promoted enrichment of younger phenotype of T cells than CD3med TSP1 and CD3hi BSP1. CD3hi TSP1 was also able to induce better cytokine production from the T cells compared to the other CD19 binders tested.

### 8.26. Example 26: Ability of CD3hi TSP1 vs. CD3hi BSP1 to Elicit T Cell Proliferation and Cytokine Production in Presence of CD19+ Target Cells

CD3hi TSP1 and CD3hi BSP1 (R variants) were evaluated for their ability to induce T cell proliferation, cytokine production and changes in T cells' surface markers expression, upon co-culture with CD19-expressing Nalm6 target cells.

### 8.26.1. Materials and Methods

Nalm-6 target cells stably expressing firefly luciferase were irradiated at 50Gy on the day of the assay set up. Peripheral blood mononuclear cells (PBMCs) previously isolated from buffy coat donors (Bern Hospital) and cryopreserved were thawed and total T cells were isolated by negative selection using the human Pan T cell Isolation Kit (Miltenyi Biotec, Cat #130-096-535) following the manufacturer's protocol. The positive fraction (called PBMCs-T cells depleted) was irradiated at 50Gy in order to be used as feeder for the co-culture. From the negative fraction, enriched in total T cells, CD8⁺ T cells were isolated by an additional step of negative selection using EasySep™ Human CD8+ T Cell Enrichment Kit (Stem Cell, Cat #19053). Untouched CD8⁺ cells were then stained with an anti-CD28 antibody (Biolegend, Cat #302922) and sorted with a FACSAria (BD) according to CD28 expression: CD8⁺CD28⁺ and CD8⁺CD28⁻. The purity of sorted cells was >95%.

After sorting, T cells were labelled with 2.5 μM of Carboxyfluorescein succinimidyl ester (CFSE, Thermo Scientific, Cat #C34554) following the manufacturer's protocol.

Each T cell subset (either CD8⁺CD28⁺ or CD8⁺CD28⁻) of CFSE-labelled T cells was co-cultured with Nalm6 target cells, seeding 50,000 T cells and 50,000 target cells to achieve an effector:target (E:T) ratio of 1:1. Cells were diluted and co-plated to obtain additional final E:T ratio of 1:3 or 1:6.

In the co-culture conditions where the presence of irradiated PBMCs-T cells depleted was required, 10,000 PBMCs were plated to obtain a 5:1 ratio, effector T cells: PBMCs.

The T cells-tumor cells co-culture was plated in a Costar 96 well plate (Corning, Cat #3585) in T Cell Media [RPMI-1640 (ThermoFisher Scientific, Cat #21875-034); 10% FBS HyClone (GE healthcare, Cat #SH30070.03); 1% Non Essential Amino Acids (Thermo Fisher Scientific, Cat #11140-050); 1% Pen/Strep (Thermo Fisher Scientific, Cat #15140122); 1% HEPES (Lonza, Cat #17737E); Sodium Pyruvate (Thermo Fisher Scientific, Cat #11360-070); 50 μM Beta-mercaptoethanol (Thermo Fisher Scientific, Cat #31350)].

CD3hi TSP1 and CD3hi BSP1, diluted in T cell Media, were added to the cells at different concentrations (1 nM, 0.1 nM and 0.01 nM) and incubated in a 5% C02, 37° C. incubator for 72 hrs. In order to be able to detect intracellular cytokines production, plates were incubated for the last 1.5 hrs of the co-culture with PMA (50 ng/ml; SIGMA, Cat #P1585)). lonomycin (500 μg/ml; Calbiochem, Cat #407950); brefeldin (10 μg/ml; Cell Signaling, Cat #9972) was also added for the last 1.5 hours of the incubation.

At the end of the 72 hrs, cells were harvested and then stained with a viability die, Zombie Aqua (Biolegend, Cat #423102) by incubating at room temperature, for 10 mins. Cells were then washed twice using FACS Buffer and stained with antibodies against surface markers: anti-CD2 (Biolegend, Cat #300214), anti-CD28 (Biolegend, Cat #302922), anti-CCR7 (Biolegend, Cat #353226), and anti-CD45RO (Biolegend, Cat #304216). Intracellular IFN-g and granzyme B (GzB) were detected by treating T cells with BD cytofix cytoperm kit (BD, Cat #555028) according to the manufacturer's instructions, and staining them with anti-IFNg (Biolegend, Cat #502509) and anti-granzyme B antibodies (BD, Cat #560213). Samples were washed with FACS buffer and acquired on a BD LSR Fortessa (BD). Analysis was performed with FLOWJO software (version 10.6.0; Tree Star Inc.).

### 8.26.2. Results

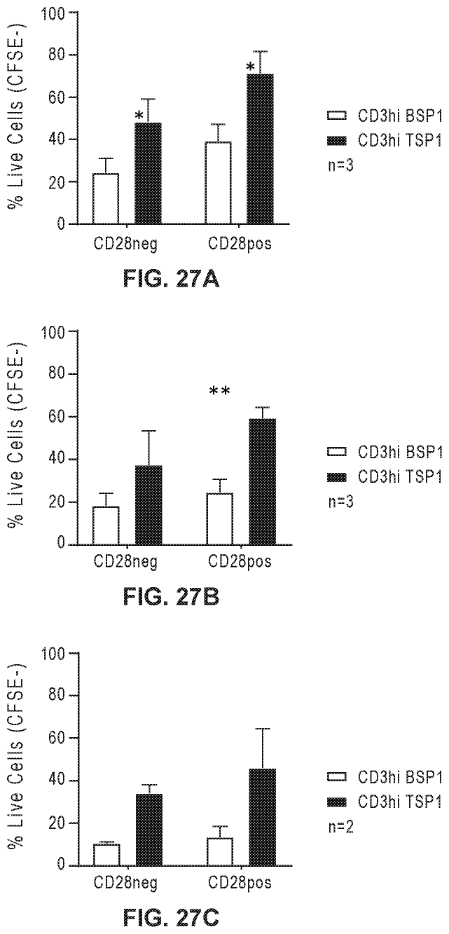

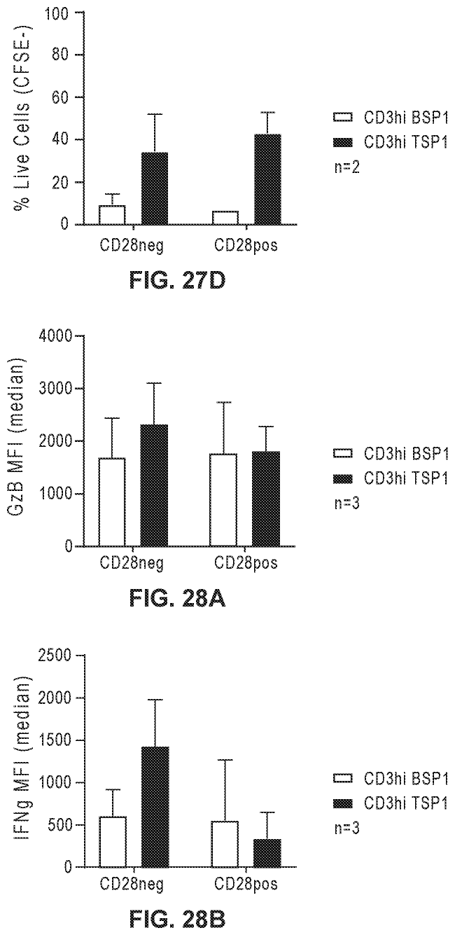

Both CD3hi TSP1 and CD3hi BSP1 induced proliferation of both CD28⁺ and CD28⁻ T cells upon co-culture with CD19 expressing target cell line Nalm6 (FIGS. 27A-27D). However, CD3hi TSP1 was more potent in inducing proliferation of both T cells subsets compared to CD3hi BSP1. The effect was observed at both concentrations tested and at the different E:T ratios used. The effect on T cell proliferation was observed both in presence or absence of irradiated PBMCs.

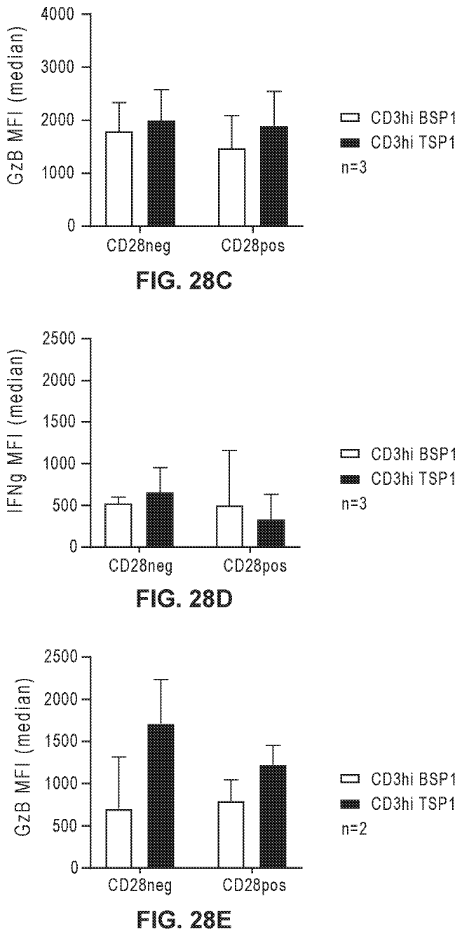

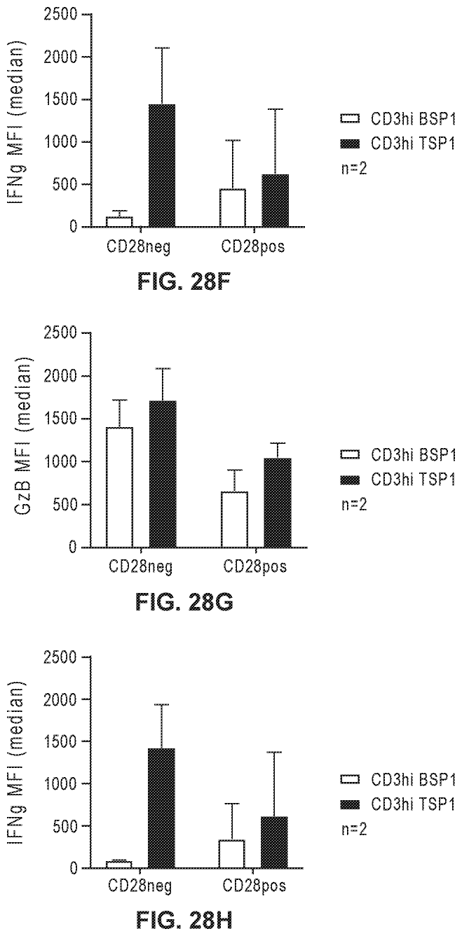

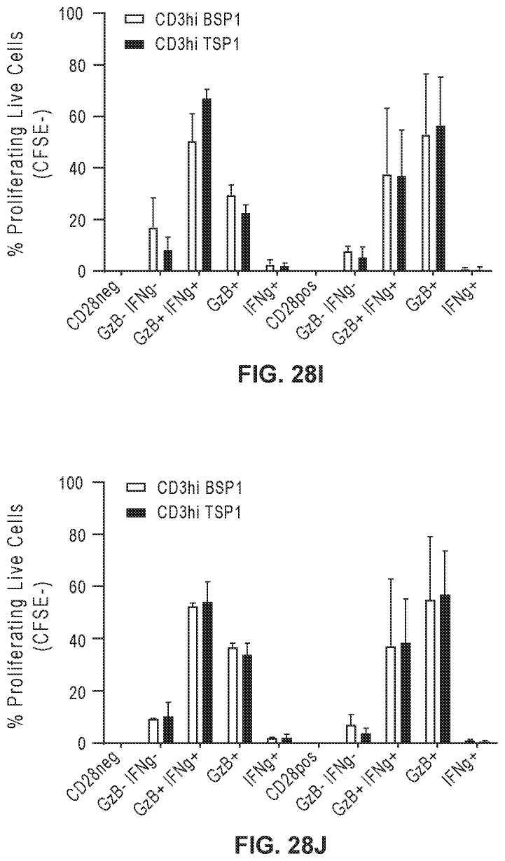

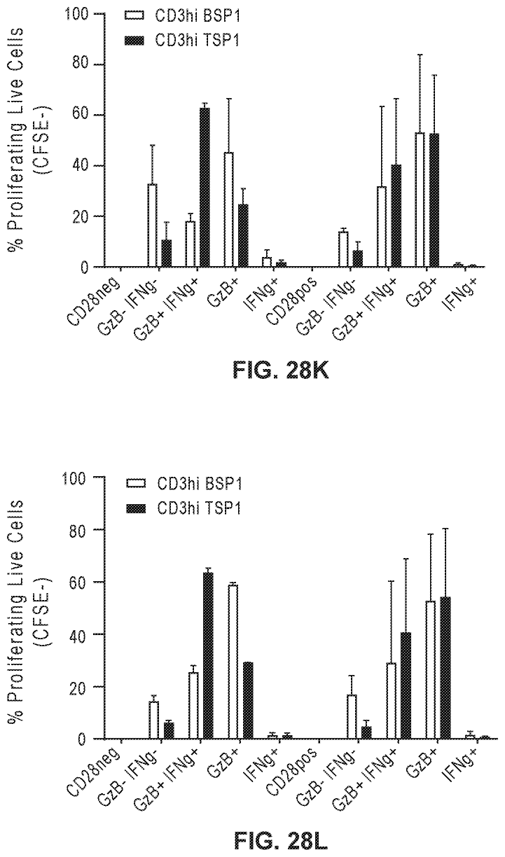

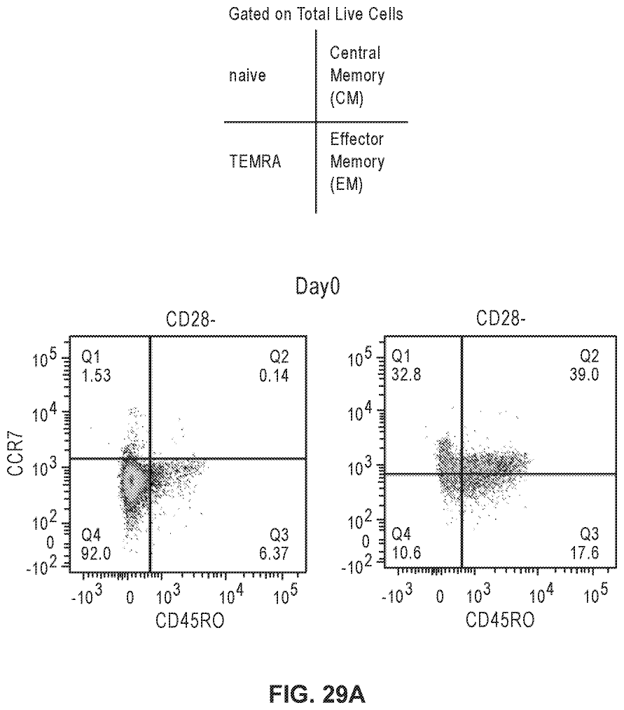

In presence of 1 nM of CD3hi BSP1, no major differences were observed in terms of percentage of T cells producing IFN-g or granzyme B (GzB); however, in presence of CD3hi TSP1 there was a clear shift in the median fluorescence intensity (MFI) for both cytokines, indicating an increase in the expression of both IFNg and GzB, in particular among the CD28⁻ T cells when co-cultured in presence of irradiated PBMCs (FIGS. 28A-28D). The effect of CD3hi TSP1 on cytokines producing-T cells is even more pronounced at 0.1 nM: both in presence and absence of irradiated PBMCs there was a clear increase in GzB⁺ T cells and IFNg⁺ T cells both within CD28− and CD28+ T cell subsets, as MFI (FIGS. 28E-28H). Moreover, the proportion (%) of CD28⁻ T cells IFNg⁺ GzB⁺ was also more pronounced in presence of CD3hiTSP1 (FIGS. 28I-28L).

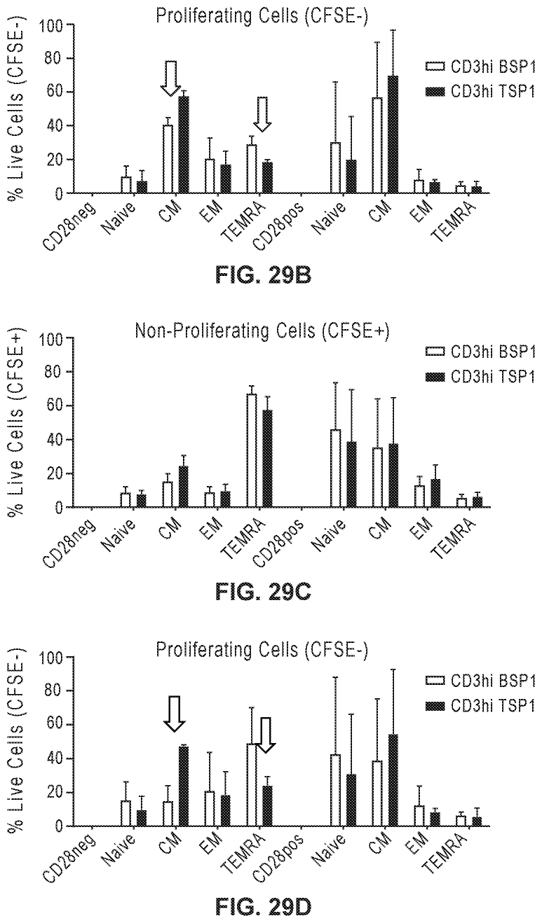

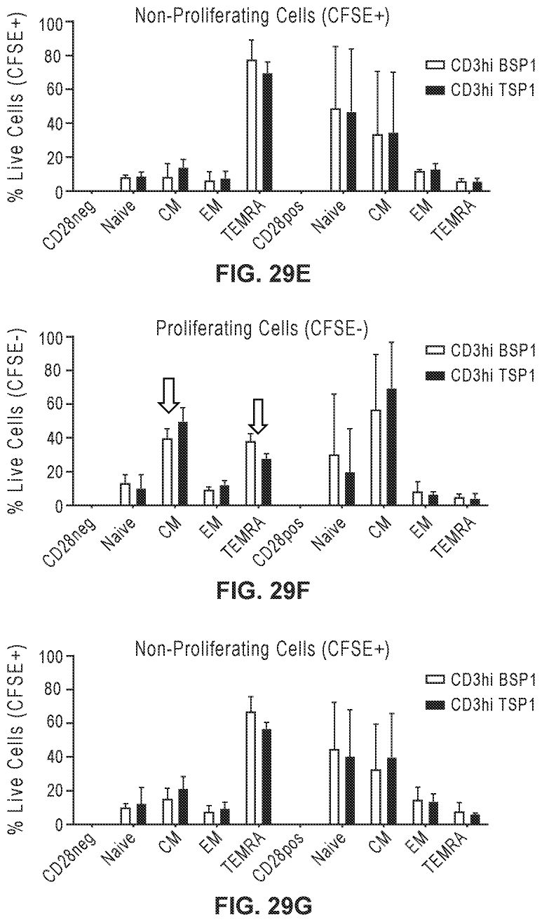

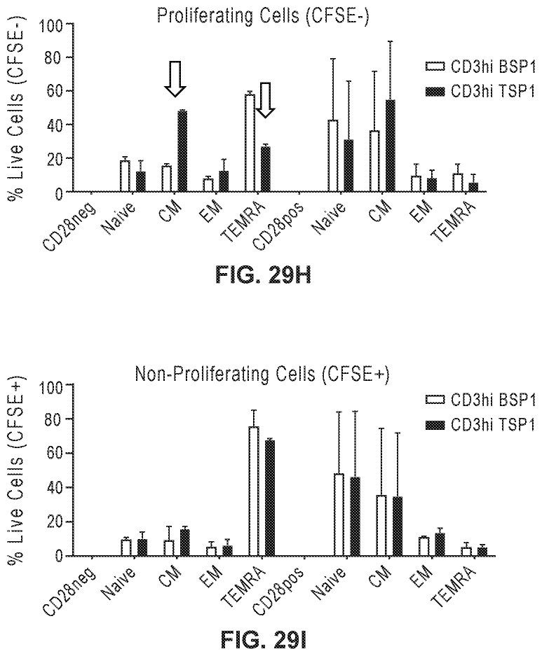

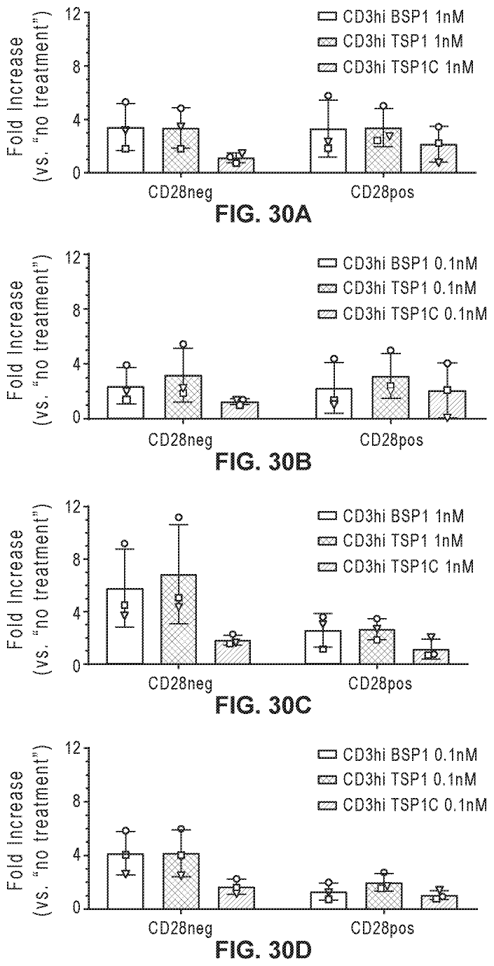

The combination of the expression profile of CD45RO and CCR7 define the distribution of the different T cell populations: naive (CD45RO⁻CCR7⁺), central memory (CM) (CD45RO⁺CCR7⁺), effector memory (EM) (CD45RO⁺CCR7⁻) and the terminally differentiated (TEMRA) (CD45RO⁻CCR7⁻). Changes in the T cell surface phenotype are shown in FIG. 29. There was no major effect of the CD3hi molecules on the CD28⁺ cells that maintain the homogenous distribution of the different T cells populations, observed right after sorting, also after 72 hrs of co-culture. Conversely, there was an effect of CD3hi TSP1 on CD28⁻ cells: while after sorting CD28⁻ cells showed almost entirely a TEMRA phenotype, after 72 hrs treatment with CD3hiTSP1 CD28− cells re-aquired a central memory/effector memory phenotype with a concomitant decrease in the proportion of cells with a more terminally differentiated (TEMRA) profile.

### 8.27. Example 27: Ability of CD3hiTSP1 vs. CD3hi BSP1 Molecules to Elicit Redirected T-Cell Cytotoxic Activity (RTCC) Against CD19+ Target Cells

An RTCC assay was set up with CD19+ Nalm6 cells, engineered to express the luciferase gene, and sorted CD8 T cells populations to measure the ability of CD3hi TSP1 and CD3hi BSP1 (R variants) to elicit cytotoxic activity of CD8 T cells subsets.

### 8.27.1. Materials and Methods

Peripheral blood mononuclear cells (PBMCs) previously isolated from buffy coat donors (Bern Hospital) and cryopreserved were thawed and total T cells were isolated by negative selection using the Pan T cell Isolation Kit, human (Miltenyi Biotec, Cat #130-096-535) following the manufacturer's protocol. The positive fraction (called PBMCs-T cell depleted) was irradiated at 50Gy in order to be used as feeder in the co-culture.

From the negative fraction, enriched in total T cells, CD8⁺ T cells were then isolated by an additional step of negative selection using EasySep™ Human CD8+ T Cell Enrichment Kit (Stem Cell, Cat #19053). Untouched CD8⁺ cells were then stained with an anti-CD28 antibody (Biolegend, Cat #302922) and sorted with a FACSAria (BD) according to the CD28 expression: CD8⁺CD28⁺ and CD8⁺CD28⁻. The purity of th sorted cells was >95%.

Each T cell subset (either CD8⁺CD28⁺ or CD8⁺CD28⁻) was then co-cultured in a 384-well flat-bottom microtiter plate (ThermoFisher Scientific, Cat #142761) with equivalent number of Nalm6 target cells to achieve an effector: target (E:T) ratio of 1:1 (3,000 T cells and 3,000 Target cells). The co-culture was set up in T Cell Media [RPMI-1640 (ThermoFisher Scientific, Cat #21875-034), 10% FBS HyClone (GE healthcare, Cat #SH30070.03), 1% Non Essential Amino Acids (Thermo Fisher Scientific, Cat #11140-050), 1% Pen/Strep (Thermo Fisher Scientific, Cat #15140122), 1% HEPES (Lonza, Cat #17737E), Sodium Pyruvate (Thermo Fisher Scientific, Cat #11360-070), 50 μM Beta-mercaptoethanol (Thermo Fisher Scientific, Cat #31350)]. Cells were diluted and co-plated to obtain additional final E:T ratio of 1:3 or 1:6. In the co-culture conditions where the presence of irradiated PBMCs-T cells depleted was required, 600 PBMCs were plated to obtain a 5:1 ratio, effector T cells: PBMCs.

CD3hi TSP1, CD3hi BSP1 and CD3hi TSP1C antibody control were added to the cells at different concentrations (1 nM, 0.1 nM and 0.01 nM).

Plates were incubated in a 37° C. incubator with 5% C02 for 72 hrs. Following the co-incubation, One-Glo (Promega, catalog #E6110) was added to all wells and the luminescence signal was subsequently measured on an ELISA Reader 4.18 1 (Biotek, Synergy H1). Target cells with One-Glo served as maximal signal. The percent RTCC of target cells was calculated using the following formula: [100−(sample/maximal signal)*100%].

### 8.27.2. Results

Results are shown in FIGS. 30A-30D. Both CD3hi TSP1 and CD3hi BSP1 mediated RTCC activity against CD19+ Nalm6-luc target cells when compared to the control antibody CD3hi TSP1C. When 0.1 nM or 1 nM of CD3hi TSP1 was used, an increase in the CD3hi TSP1-mediated RTCC was observed in the settings with CD8⁺CD28⁻ T cells (in presence of irradiated feeder) compared to the other treatments.

### 8.28. Example 28: Anti-Tumor Activity of CD3hi TSP1 and CD3med TSP1 in an Adoptive Transfer Adaptation of the OCI-LY-19 Diffuse Large B-Cell Lymphoma Tumor Model in NSG Mice

The anti-tumor activity of CD3hi TSP1 and CD3med TSP1 (H variants) were studied in an OCI-LY-19 diffuse large B-cell lymphoma (DLBCL) tumor model in NSG mice.

### 8.28.1. Materials and Methods

On Day 0, OCI-LY-19 cells were harvested and suspended in Hanks Balanced Salt Solution (HBSS) at a concentration of 500×10⁶ cells/mL. Female NOD.Cg-Prkdcscid Il2rgtm1Wjl/SzJ mice (NSG mice) at −6 weeks old (Jackson Laboratories, ME) were injected with 5×10⁶ OCI-LY-19 cells in 200 μL subcutaneously on the right flank. Seven days following tumor inoculation, each mouse received an adoptive transfer (AdT) of 15×10⁶ of peripheral blood mononuclear cells (PBMCs) in 100 μL via IV injection in the lateral tail vein. The PBMCs were previously isolated from a human leukopak, frozen and stored in Cryostor10 media in vapor phase liquid nitrogen tank until use. Immediately prior to AdT, PBMCs were thawed and suspended at a concentration of 100×10⁶cells/ml in Hanks Balanced Salt Solution (HBSS). When tumor burden (TB) reached an average of −200 mm³ volume measured via mechanical caliper, mice (n=8/group) were treated with a single IV administration of CD3hi TSP1 or CD3med TSP1 at dose levels 0.003 mg/kg, 0.01 mg/kg, 0.03 mg/kg, 0.1 mg/kg or 0.3 mg/kg. Anti-tumor activity of each antibody was compared to an untreated control group that received tumor implant and AdT but no treatment (tumor+AdT) (Table 26). The tumor only group was included to meter the allogeneic response observed with untreated control. All treatments were administered at 10 mL/kg according to individual mouse body weights. Anti-tumor activity was determined by percent change in tumor burden vs. change in untreated control (% ΔT/ΔC) or % regression.

Tumor burden and body weights were recorded twice weekly. Tumor burden was measured by bioluminescence signal intensity in p/s using a bioluminescence imaging system (IVIS200, Perkin Elmer). Anti-tumor activity was determined by % ΔT/ΔC using the formula: 100×ΔTB_{treatment, time}/ΔTB_{control group, time} if ΔTB≥0; or % regression: (−1×(100×(TB_{final}−TBᵢₙᵢₜᵢₐₗ/TBᵢₙᵢₜᵢₐₗ) if ΔTV<0. TBᵢₙᵢₜᵢₐₗ is the tumor burden on the day of treatment initiation. % ΔT/ΔC values<42% were considered to have anti-tumor activity. Percent body weight change was determined using the formula: 100×((BWₜᵢₘₑ−BWᵢₙᵢₜᵢₐₗ)/BWᵢₙᵢₜᵢₐₗ). Statistical analysis using One-way ANOVA with Dunnett's multiple comparison test was performed using Graphpad Prism Software, Version. 7.03.

On day 25 following OCI-LY-19 implantation, all animals from the untreated control group were euthanized due to tumor burden.

### 8.28.2. Results

This study had minimal allogeneic response (FIGS. 31A-31B).

Antibody treatment with CD3hi TSP1 at 0.1 mg/kg and 0.3 mg/kg resulted in significant tumor regressions of 72.41% and 84.50%, respectively. Antibody treatment with CD3hi TSP1 at 0.03 mg/kg resulted in tumor regression of 13.74%. Antibody treatment with CD3hi TSP1 at 0.003 mg/kg exhibited significant anti-tumor activity at 1.38% ΔT/ΔC. Antibody treatment with CD3hi TSP1 at 0.003 mg/kg dose level was not active in this model (Table 26; FIG. 31A).

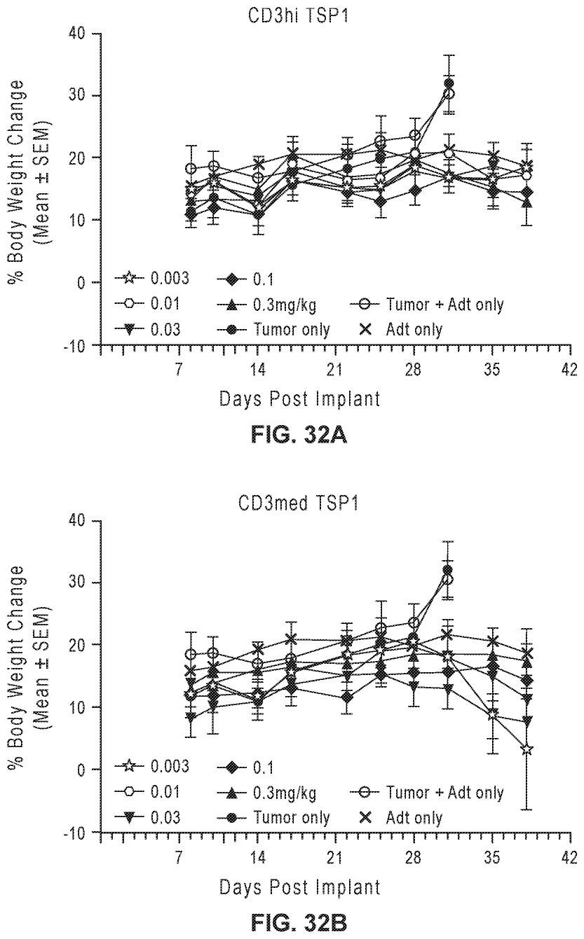

There was no antibody associated body weight loss with CD3hi TSP1. The body weight change observed with the treatment of CD3hi TSP1 was most likely due to the onset of graft-versus host disease (GvHD). Body weight loss is an endpoint parameter for both disease burden and onset of GvHD. At 35-42 days post-PBMC injection (28-35 days post-tumor implant), animals began to exhibit weight loss attributed to GvHD. Animals with high tumor burden also demonstrated disease-burden associated weight loss. Over the course of the study, body weights increased relative the initial measurement taken on the day of tumor implant (Table 26, FIG. 32A). However at the end of study, the body weight loss observed relative to the peak gain is indicative of GvHD and disease-burden induced weight loss.

Antibody treatment with CD3med TSP1 resulted in significant anti-tumor responses at 0.1 mg/kg (5.60% regression) and 0.3 mg/kg (36.33% regression). Treatment with CD3med TSP1 resulted in significant anti-tumor responses with % ΔT/ΔC values of 7.39% for the 0.03 mg/kg dose level. Antibody treatment with CD3med TSP1 at 0.003 and 0.01 mg/kg was not active in this model (Table 27; FIG. 31B).

There was no antibody associated body weight loss with CD3med TSP1. Body weight loss due to the onset of GvHD was not observed for this construct by the end of the study (Table 27, FIG. 32B).

TABLE 26

| | | | | | | Host Response | |
|---|---|---|---|---|---|---|---|
| | | | Tumor Response | | | Δ Body | |
| | | | | | Δ Tumor burden | weight from | |
| | | | | | from post-dose | post-dose | |
| | | | | | (mm³) | (%) | Survival |
| Test | Dose | | ΔT/ΔC | Regression | (Mean) | (Mean ± SEM) | (survivors/ |
| agent | (mg/kg) | Schedule | (%) | (%) | Day 25 | Day 25 | total) |
| Tumor | N/A | — | 100 | — | 952.252 | | 5/5 |
| only | | | | | | | |
| Tumor + | N/A | — | 100 | — | 822.216 | | 8/8 |
| AdT | | | | | | | |
| CD3hi | 0.003 | Single | 24.20 | — | 198.9588 | 1.7 ± 1.6 | 8/8 |
| TSP1 | | dose/IV | | | | | |
| CD3hi | 0.01 | Single | 1.38 | — | 11.37875 | −0.7 ± 1.8 | 8/8 |
| TSP1 | | dose/IV | | | | | |
| CD3hi | 0.03 | Single | — | 13.74* | −29.7413 | 1.9 ± 2.0 | 8/8 |
| TSP1 | | dose/IV | | | | | |
| CD3hi | 0.1 | Single | — | 72.41* | −155.988 | 1.2 ± 2.3 | 8/8 |
| TSP1 | | dose/IV | | | | | |
| CD3hi | 0.3 | Single | — | 84.50* | −187.737 | −0.4 ± 1.0 | 7/7** |
| TSP1 | | dose/IV | | | | | |

*p < 0.05, Dunnett's multiple comparison test
**One animal was excluded from the study

TABLE 27

| | | | Tumor Response | | | Host Response | |
|---|---|---|---|---|---|---|---|
| | | | | | Δ Tumor burden | Δ Body | |
| | | | | | from initial | weight from | |
| | | | | | (mm³) | initial | Survival |
| Test | Dose | | ΔT/ΔC | Regression | (Mean) | (%) | (survivors/ |
| agent | (mg/kg) | Schedule | (%) | (%) | Day 25 | (Mean ± SEM) | total) |
| CD3med | 0.003 | Single | 45.36 | — | 372.94 | 4.2 ± 2.1 | 8/8 |
| TSP1 | | dose/IV | | | | | |
| CD3med | 0.01 | Single | 57.13 | — | 469.6988 | 5.7 ± 2.7 | 8/8 |
| TSP1 | | dose/IV | | | | | |
| CD3med | 0.03 | Single | 7.39 | — | 60.7375 | 2.6 ± 1.6 | 8/8 |
| TSP1 | | dose/IV | | | | | |
| CD3med | 0.1 | Single | — | 5.60* | −12.1488 | 3.3 ± 2.0 | 8/8 |
| TSP1 | | dose/IV | | | | | |
| CD3med | 0.3 | Single | — | 36.33* | −78.7388 | 3.54 ± 1.4 | 8/8 |
| TSP1 | | dose/IV | | | | | |

*p < 0.05, Dunnett's multiple comparison test

### 8.29. Example 29: Anti-Tumor Activity Following Multiple Doses of CD3 TSP1, CD3hi BSP1 and CD3med TSP1 in the OCI-LY-19 in the Adaptation of a DLBCL Subcutaneous Tumor Model in huCD34+ NSG Mice

The anti-tumor activity of CD3hi TSP1, CD3hi BSP1, and CD3med TSP1 (H variants) were studied in an OCI-LY-19 DLBCL subcutaneous tumor model in huCD34+ NSG mice.

### 8.29.1. Materials and Methods

The process of humanization of NGS mice used in this study is shown schematically in in FIG. 33. Briefly, female female NSG mice at ˜6 weeks old (Jackson Laboratories, ME) underwent a preconditioning protocol to depopulate the bone marrow niche. This was accomplished by either chemical ablation or by X-ray irradiation to allow for reconstitution of a human immune system in each NSG mouse. Within twenty-four hours following preconditioning, 50,000 huCD34+ stem cells (huCD34+SC) isolated from single umbilical cords (Lonza, StemCell) were introduced in 100 μL via IV injection in the lateral tail vein. Each mouse received huCD34+SC from a single donor. The huCD34+SC were received frozen and stored in a −200° C. liquid nitrogen tank until use. Immediately prior to inoculation, huCD34+SC vials were removed from the liquid nitrogen tank, thawed in a bead-bath at 37° C. and re-suspended in PBS at a final concentration of 500,000 cells/mL. For sixteen weeks post humanization, mice were monitored weekly for body weights and body condition. At week 16, mice were bled via the tail and human immune reconstitution (human engraftment) was ascertained by fluorescent activated cell sorting (FACS). Mice with ≥25% hCD45/total CD45 were considered stably engrafted and were eligible for study enrollment.

Following engraftment assessment, mice were implanted with tumor cells subcutaneously. On Day 0, OCI-LY-19 cells were harvested and suspended in Hanks Balanced Salt Solution (HBSS) at a concentration of 10×10⁷ cells/mL and then diluted 1:1 with matrigel to give a final concentration of 5×10⁷ cells/mL. Mice were implanted via subcutaneous (SQ) injection on the right flank with 5×10⁶ cells/mouse in 100 μL volume. Fifteen days post implant (mean tumor volume ˜250-300 mm³ measured via calipers), mice were randomized on two parameters: donor and tumor volume. This ensured equal distribution of donors and comparable tumor volumes in each group. There were 3 treatment groups, n=8, and untreated control, n=5. Mice were treated weekly for 2-4 weeks via IV administration with CD3hi TSP1 (0.3 mg/kg), CD3med TSP1 (1.0 mg/kg), or CD3hi BSP1 (0.3 mg/kg). Anti-tumor activity of each antibody was compared to an untreated huCD34+SC control group that received tumor implant (tumor+CD34+) (Table 28). All treatments were administered at 10 mL/kg according to individual mouse body weights. Anti-tumor activity was determined by percent change in tumor volume of treated vs. untreated control (% ΔT/ΔC) or % regression and durability of response was evaluated by monitoring % surviving animals over time. Animals whose TV, BW or BCS (body condition score) that reached end point criteria by exceeding limits provisioned in the lab's animal use protocol (AUP) were euthanized.

Tumor burden (TV) and body weights were recorded twice weekly. Tumor burden was measured by calipers, capturing length and width, and the tumor volume was calculated using the formula (w²×L)/3.14. Body weight was measured by scale. Both parameters were entered into an in-house system (INDIGO). Anti-tumor activity was determined by % ΔT/ΔC using the formula: 100×ΔTB_{treatment, time}/ΔTB_{control group, time} if ΔTB≥0; or % regression: (−1×(100×(TB_{final}−TBᵢₙᵢₜᵢₐₗ/TBᵢₙᵢₜᵢₐₗ) if ΔTV<0. TBᵢₙᵢₜᵢₐₗ is the tumor burden on the day of treatment initiation. % ΔT/ΔC values<42% were considered to have anti-tumor activity. Percent body weight change was determined using the formula: 100×((BWₜᵢₘₑ−BWᵢₙᵢₜᵢₐₗ)/BWᵢₙᵢₜᵢₐₗ). Statistical analysis using One-way ANOVA with Dunnett's multiple comparison test was performed using Graphpad Prism Software, Version. 7.03 (Day 24 post implant).

In addition, Time to endpoint was evaluated using KAPLAN-Meyer survival graph and analysis using Graphpad Prism Software, Version. 7.03, and was performed to compare differences in median time to endpoint (TTE). Mice which achieved tumor endpoint when tumor volume exceeded 1200 mm³ and mice euthanized for reasons besides tumor volume related to disease progression, such as ulceration, metastasis, body weight loss or poor body condition were scored as dead (“1”). Animals euthanized for reasons other than tumor progression, such as adverse drug events, were censored (“0”). Log-Rank (Mantel-Cox) survival analysis was performed, and all pairwise multiple comparison procedures were performed using Holm-Sidak method with an overall significance level P<0.05 (SigmaPlot 13.0). Graphical analysis of TTE was performed in Prism (GraphPad v7.03). Individual response criteria were also evaluated and scored as either Complete Response (CR), no detectable tumor at time of last measurement; Partial Response (PR), tumor volume less than baseline measurement at any time point followed by regrowth; or No Response (NR), tumor continues to increase over baseline measurement throughout the study. The last day of the study was captured at Day 39.

On day 24 following OCI-LY-19 implantation, all animals from the untreated control group were euthanized due to tumor burden. Statistical analysis was evaluated on Day 24.

### 8.29.2. Results

Treatment with all three antibodies showed significant differences in tumor activity compared to the tumor+CD34⁺ control group. CD3hi TSP1 at 0.3 mg/kg resulted in significant tumor regressions of 47.4% whereas the CD3hi BSP1 did not achieve regressions (16.3% ΔT/ΔC). Treatment with CD3med TSP1 at 1.0 mg/kg resulted in tumor regressions 64.5% (Table 28, FIG. 34A).

There was treatment associated body weight loss with CD3hi TSP1, CD3med TSP1, and CD3hi BSP1 observed following the first dose only. The severity of body weight loss was impacted by donor as well, with different donors showing variable peak body weight loss. Without being bound by theory, the body weight change observed following the first dose is hypothesized to be target-mediated driven and exacerbated by the depletion of the inherent B cells. Body weight loss is an endpoint parameter for both disease burden and treatment induced responses. Animals with high tumor burden demonstrated disease-burden associated weight loss. Over the course of the study, body weights were observed to increase relative the initial measurement taken on the day of tumor implant, but decrease in response to the progressing disease burden (Table 28, FIG. 34B).

TABLE 28

| | | | | | | Host Response | |
|---|---|---|---|---|---|---|---|
| | | | Tumor Response | | | Δ Body | |
| | | | | | Δ Tumor burden | weight from | |
| | | | | | from initial | initial | |
| | | | | | (mm³) | (%) | Survival |
| Test | Dose | | ΔT/ΔC | Regression | (Mean ± SEM) | (Mean ± SEM) | (survivors/ |
| agent | (mg/kg) | Schedule | (%) | (%) | Day 24 | Day 24 | total) |
| Tumor + | N/A | — | — | — | 1733.60 ± 130 | −5.12 ± 4.5 | 5/5 |
| CD34⁺ | | | | | | | |
| CD3hi | 0.3 | QWx3 | — | 47.4* | −175.93 ± 76.8 | −13.51 ± 2.15 | 8/8 |
| TSP1 | | dose/IV | | | | | |
| CD3hi | 0.3 | QWx3 | 16.25* | | 281.69 ± 292.5 | −5.12 ± 1.7 | 8/8 |
| BSP1 | | dose/IV | | | | | |
| CD3med | 1.0 | QWx3 | — | 64.3* | −215.32 ± 38.48 | −11.30 ± 1.93 | 8/8 |
| TSP1 | | | | | | | |

*p < 0.05, Dunnett's multiple comparisons test

### 8.30. Example 30: Anti-Tumor Activity in a Single Dose, Dose Range Finding Study Comparing CD3hi TSP1 and CD3med TSP1 in a DLBCL Subcutaneous Tumor Model in huCD34+ NSG Mice

The anti-tumor activity of CD3hi TSP1, CD3hi BSP1, and CD3med TSP1 (H variants) were studied in an OCI-LY-19 DLBCL subcutaneous tumor model in huCD34+ NSG mice.

### 8.30.1. Materials and Methods

Female humanized CD34+ NOD.Cg-Prkdcscid Il2rgtm1Wjl/SzJ mice (HuNSG mice) were purchased from Jackson Laboratories (Sacramento, CA). Mice were humanized using cord blood.

The engraftment levels of hCD45+ cells were determined prior to shipment and confirmed in-house prior to the start of the study. HuNSG mice that had over 25% hCD45+ cells in the peripheral blood were considered as engrafted and humanized. HuNSG derived from different donors with different levels of engraftment were randomized into every treatment group in the study.

Following engraftment assessment, mice were implanted with tumor cells subcutaneously. On Day 0, OCI-LY-19 cells were harvested and suspended in Hanks Balanced Salt Solution (HBSS) at a concentration of 10×10⁷ cells/mL and then diluted 1:1 with matrigel to give a final concentration of 5×10⁷ cells/mL. Mice were implanted via subcutaneous (SQ) injection on the right flank with 5×10⁶ cells/mouse in 100 μL volume. Nine days post implant, (mean tumor volume ˜250-300 mm³ measured via calipers) mice were randomized on two parameters: donor and tumor volume. This ensured equal distribution of donors and comparable tumor volumes in each group. There were 11 groups with n=8 treatment group and n=5 in the untreated control. Mice were administered a single dose via IV administration with CD3hi TSP1 or CD3med TSP1 across the following dose range 1.0 mg/kg, 0.3 mg/gk, 0.1 mg/kg and 0.01 mg/kg. Anti-tumor activity of each antibody was compared to an untreated huCD34+SC control group that received tumor implant (tumor+CD34+) (Table 29). All treatments were administered at 10 mL/kg according to individual mouse body weights. Anti-tumor activity was determined by percent change in tumor volume of treated vs. untreated control (% ΔT/ΔC) or % regression and durability of response was evaluated by monitoring % surviving animals overtime. Animals whose TV, BW or BCS (body condition score) that reached end point criteria by exceeding limits provisioned in the lab's animal use protocol (AUP) were euthanized.

Tumor burden (TV) and body weights were recorded twice weekly. Tumor burden was measured by calipers, capturing length and width, and the tumor volume was calculated using the formula (w²×L)/3.14. Body weight was measured by scale. Both parameters were entered into an in-house system (INDIGO). Anti-tumor activity was determined by % ΔT/ΔC using the formula: 100×ΔTB_{treatment, time}/ΔTB_{control group, time} if ΔTB≥0; or % regression: (−1×(100×(TB_{final}−TBᵢₙᵢₜᵢₐₗ/TBᵢₙᵢₜᵢₐₗ) if ΔTV<0. TBᵢₙᵢₜᵢₐₗ is the tumor burden on the day of treatment initiation. (% ΔT/ΔC values<42% were considered to have anti-tumor activity). Percent body weight change was determined using the formula: 100×((BWₜᵢₘₑ−BWᵢₙᵢₜᵢₐₗ)/BWᵢₙᵢₜᵢₐₗ). Statistical analysis using One-way ANOVA with Dunnett's multiple comparison test was performed using Graphpad Prism Software, Version. 7.03. In addition, durability of response was evaluated using KAPLAN-Meyer survival graph and analysis using Graphpad Prism Software, Version. 7.03.

On day 24 following OCI-LY-19 implantation, all animals from the untreated control group were euthanized due to tumor burden. Statistical analysis was evaluated on Day 24.

### 8.30.2. Results

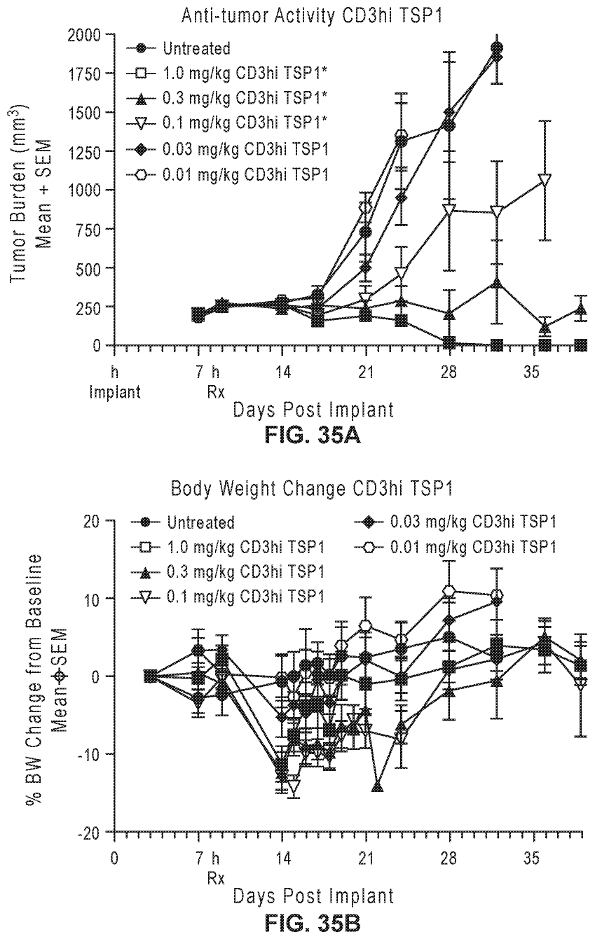

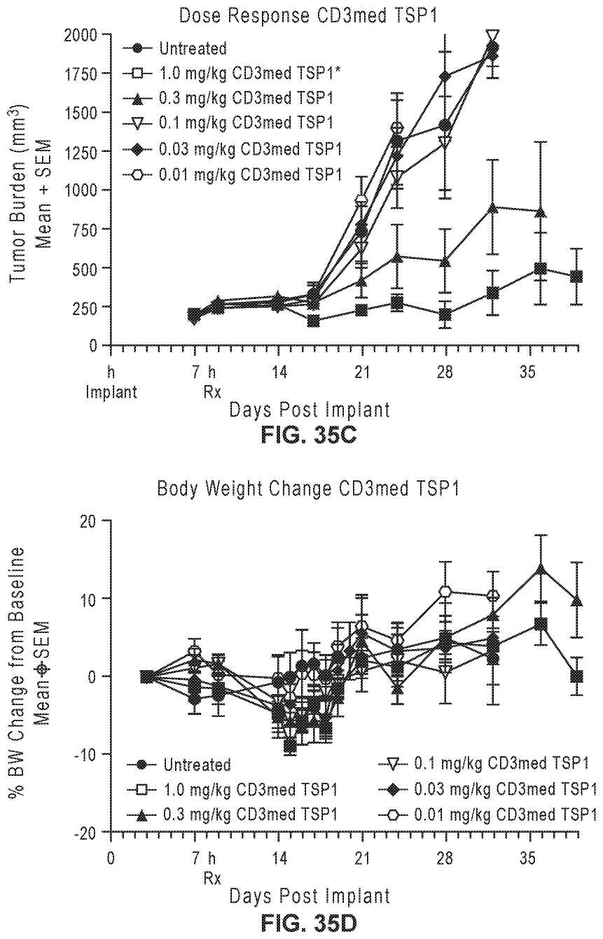

There was a statistical significant difference in tumor activity observed with the 1.0 mg/kg, 0.3 mg/kg and 0.1 mg/kg doses of CD3hi TSP compared to the tumor+CD34⁺ control group. CD3hi TSP1 at 1.0 mg/kg resulted in significant tumor regressions of 35.3%, whereas CD3hi TSP1 at 0.3 mg/kg and at 0.1 mg/kg showed robust significant anti-tumor activity with ΔT/ΔC values of 0.05% and 19.5%, respectively. The dose levels administered below 0.1 mg/kg did not achieve anti-tumor responses, with the 0.03 mg/kg dose of CD3hi TSP1 having a ΔT/ΔC value of 65.8% and the 0.01 mg/kg dose having a ΔT/ΔC value of 100% (Table 29, FIG. 35A).

Antibody treatments with CD3med TSP1 resulted in significant anti-tumor activity. CD3med TSP1 dosed at 1.0 mg/kg achieved significant tumor response with a ΔT/ΔC of 0.05%. The 0.3 mg/kg dose level of CD3med TSP1 did show anti-tumor activity (ΔT/ΔC: 26.9<42) but was not significant compared to the control. Doses lower that 0.3 mg/kg did not show significant tumor activity with ΔT/ΔC values of 79.8%, 90.3%, and 100% for 0.1 mg/kg, 0.03 mg/kg and 0.01 mg/kg doses, respectively (Table 29, FIG. 35C).

There was treatment associated body weight loss observed across multiple dose levels following administration of CD3hi TSP1 and CD3med TSP1. The severity of body weight loss was a combination of both donor and dose level effects, with different donors showing variable peak body weight loss. Without being bound by theory, the body weight change observed following the dose is hypothesized to be target-mediated driven and exacerbated by the depletion of the inherent B cells. Body weight loss is an endpoint parameter for both disease burden and treatment induced responses. Animals with high tumor burden demonstrated disease-burden associated weight loss. Over the course of the study, body weights were observed to increase relative to the initial measurement taken on the day of tumor implant (Table 29, FIGS. 35B and 35D), but decrease in response to the progressing disease burden.

TABLE 29

| | | | | | | Host Response | |
|---|---|---|---|---|---|---|---|
| | | | Tumor Response | | | Δ Body | |
| | | | | | Δ Tumor burden | weight from | |
| | | | | | from initial | initial | Survival |
| | | | | | (mm³) | (%) | (survivors/ |
| Test | Dose | | ΔT/ΔC | Regression | (Mean ± SEM) | (Mean ± SEM) | total) |
| agent | (mg/kg) | Schedule | (%) | (%) | Day 24 | Day 24 | Day 24 |
| Untreated | N/A | — | — | — | 1057. ± 304.2 | 6.02 ± 1.72 | 5/5 |
| control | | | | | | | |
| CD3hi | 1.0 | Single | — | 35.3* | −91.6 ± 13.8 | −1.96 ± 2.31 | 8/8 |
| TSP1 | | dose/IV | | | | | |
| CD3hi | 0.3 | Single | 0.05* | — | −0.57 ± 95.0 | −6.09 ± 2.34 | 8/8 |
| TSP1 | | dose/IV | | | | | |
| CD3hi | 0.1 | Single | 19.5* | — | 205.9 ± 170.6 | −7.06 ± 3.02 | 8/8 |
| TSP1 | | dose/IV | | | | | |
| CD3hi | 0.03 | Single | 65.8 | — | 695.0 ± 168.4 | −3.32 ± 1.63 | 8/8 |
| TSP1 | | dose/IV | | | | | |
| CD3hi- | 0.01 | Single | 100 | — | 1110.7 ± 201.7 | 4.54 ± 2.25 | 8/8 |
| TSP1 | | dose/IV | | | | | |
| CD3med | 1.0 | Single | 0.05* | — | 35.5 ± 57.6 | 3.1 ± 2.5 | 6/8 |
| TSP1 | | dose/IV | | | | | |
| CD3med | 0.3 | Single | 26.9 | — | 284.0 ± 200.9 | −3.0 ± 2.2 | 8/8 |
| TSP1 | | dose/IV | | | | | |
| CD3med | 0.1 | Single | 79.8 | — | 843.2 ± 196.5 | 0.3 ± 2.5 | 8/8 |
| TSP1 | | dose/IV | | | | | |
| CD3med | 0.03 | Single | 90.3 | — | 954.8 ± 180.1 | 4.8 ± 3.7 | 8/8 |
| TSP1 | | dose/IV | | | | | |
| CD3med | 0.01 | Single | 100 | — | 1139.3 ± 155.7 | 12.7 ± 1.8 | 8/8 |
| TSP1 | | dose/IV | | | | | |

*p < 0.05, Dunnett's multiple comparison test

### 8.31. Example 31: Anti-Tumor Activity of CD3hi BSP1, CD3hi TSP1, and CD3med TSP1 in an Adoptive Transfer Adaptation of the Daudi-Luc Burkitt's Lymphoma Subcutaneous Tumor Model in NSG Mice

The anti-tumor activity of CD3hi BSP1, CD3hi TSP1, and CD3med TSP1 (H variants) were studied in an adoptive transfer adaptation of the Daudi-Luc Burkitt's lymphoma subcutaneous tumor model in NSG mice.

### 8.31.1. Materials and Methods

On Day 0, Daudi-Luc cells were harvested and suspended in a 1:1 mixture of Hanks Balanced Salt Solution (HESS) and Matrigel at a concentration of 50×10⁶ cells/mL. Female NSG mice at ˜6 weeks old (Jackson Laboratories, ME) were injected with 5×10⁶ Daudi-Luc cells in 100 μL subcutaneously (SQ) in the right flank. Three days following tumor inoculation, each mouse received an adoptive transfer (AdT) of 15×10⁶ of peripheral blood mononuclear cells (PBMCs) in 100 μL via intravenous (IV) injection in the lateral tail vein. The PBMCs were previously isolated from a human leukopak, frozen and stored in Cryostor10 media in vapor phase liquid nitrogen tank until use. Immediately prior to AdT, PBMCs were thawed and suspended at a concentration of 150×10⁶cells/ml in Hanks Balanced Salt Solution (HBSS). When tumor volume (TV) reached an average of ˜250 cubic millimeters (mm³) measured via caliper (Day 10 post-implant), mice (n=8/group) were treated with a single IV administration of CD3hi BSP1, CD3hi TSP1, or CD3med TSP1 at dose levels of 1.0 mg/kg, 0.3 mg/kg, or 0.1 mg/kg. Anti-tumor activity of each antibody was compared to an untreated control group that received tumor implant and AdT but no treatment (tumor+AdT) (Table 30). The tumor only group was included to meter the allogeneic response observed with untreated control. All treatments were administered at 10 mL/kg according to individual mouse body weights. Anti-tumor activity was determined by percent change in tumor volume vs. change in untreated control (% ΔT/ΔC) or % regression.

Tumor volume and body weights were recorded twice weekly. Tumor volume was measured by caliper. Anti-tumor activity was determined by % ΔT/ΔC using the formula: 100×ΔTV_{treatment, time}/ΔTV_{control group, time} if ΔTV≥0; or % regression: (−1×(100×(TV_{final}−TVᵢₙᵢₜᵢₐₗ/TVᵢₙᵢₜᵢₐₗ) if ΔTV<0. TVᵢₙᵢₜᵢₐₗ is the tumor volume on the day of treatment initiation. % ΔT/ΔC values<42% were considered to have anti-tumor activity. Percent body weight change was determined using the formula: 100×((BWₜᵢₘₑ−BWᵢₙᵢₜᵢₐₗ)/BWᵢₙᵢₜᵢₐₗ). Statistical analysis using One-way ANOVA with Dunnett's multiple comparison test was performed using Graphpad Prism Software, Version. 7.03.

On day 36 following Daudi-Luc implantation, 25% of animals from the Tumor+AdT control group were euthanized due to tumor volume.

### 8.31.2. Results

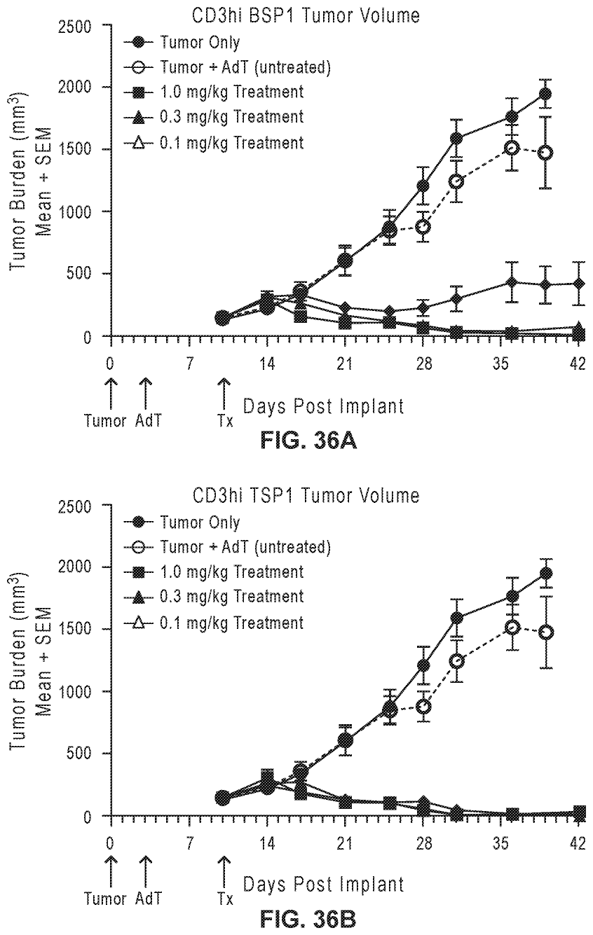

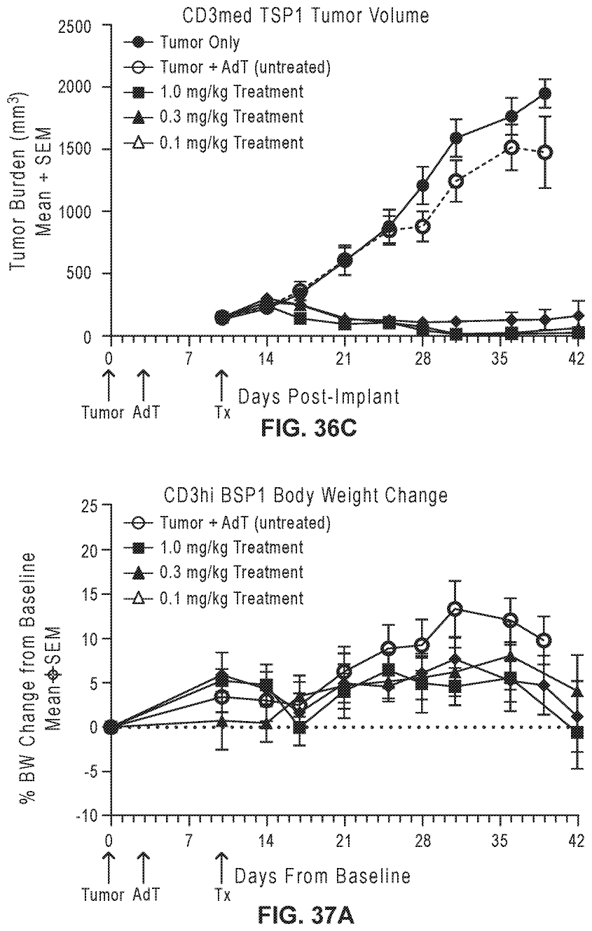

This study had minimal allogeneic response (FIGS. 36A-36C).

Antibody treatment with CD3hi BSP1 at 1.0 mg/kg and 0.3 mg/kg resulted in significant tumor regressions of 85.21% and 73.26%, respectively. Antibody treatment with CD3hi BSP1 at 0.1 mg/kg exhibited significant anti-tumor activity (20.89% ΔT/ΔC value). Antibody treatment with CD3med TSP1 resulted in significant anti-tumor responses at all three dose levels: 1.0 mg/kg (90.86% regression), 0.3 mg/kg (85.13% regression), and 0.1 mg/kg (13.51% regression). Antibody treatment with CD3hi TSP1 resulted in significant tumor regressions at all three dose levels: 1.0 mg/kg (90.08% regression), 0.3 mg/kg (91.86% regression), and 0.1 mg/kg (87.52% regression).

There was no antibody associated body weight loss with any of the three constructs tested. Without being bound by theory, the body weight change observed at approximately Day 35 from baseline was most likely due to the onset of graft-versus host disease (GvHD). Body weight loss is an endpoint parameter for onset of GvHD. At 32-39 days post-PBMC injection (35-42 days post-tumor implant), animals began to exhibit weight loss attributed to GvHD. Over the course of the study, body weights increased relative the initial measurement taken on the day of tumor implant (Table 30, FIGS. 37A-37C). However at the end of study, the body weight loss observed relative to the peak gain is indicative of GvHD-induced weight loss.

TABLE 30

| | | | | | | Host Response | |
|---|---|---|---|---|---|---|---|
| | | | Tumor Response | | | Δ Body | |
| | | | | | Δ Tumor burden | weight from | |
| | | | | | from initial | initial | |
| | | | | | (mm³) | (%) | Survival |
| Test | Dose | | ΔT/ΔC | Regression | (Mean) | (Mean ± SEM) | (survivors/ |
| agent | (mg/kg) | Schedule | (%) | (%) | Day 36 | Day 36 | total) |
| Tumor | N/A | — | 119.42 | — | 1633.79 | 11.653 ± 1.871 | 4/8 |
| only | | | | | | | |
| Tumor + | N/A | — | 100.00 | — | 1368.13 | 8.435 ± 2.241 | 8/8 |
| Adt | | | | | | | |
| (Untreated | | | | | | | |
| Control) | | | | | | | |
| CD3hi | 1.0 | Single | — | 85.21 | −124.67 | 0.296 ± 3.360 | 8/8 |
| BSP1 | | dose/IV | | | | | |
| CD3hi | 0.3 | Single | — | 73.26 | −107.15 | 8.394 ± 6.267 | 8/8 |
| BSP1 | | dose/IV | | | | | |
| CD3hi | 0.1 | Single | 20.89 | — | 285.84 | −0.359 ± 2.569 | 8/8 |
| BSP1 | | dose/IV | | | | | |
| CD3hi | 1.0 | Single | — | 90.08 | −131.77 | 5.423 ± 2.220 | 8/8 |
| TSP1 | | dose/IV | | | | | |
| CD3hi | 0.3 | Single | — | 91.86 | −134.39 | 2.254 ± 2.975 | 8/8 |
| TSP1 | | dose/IV | | | | | |
| CD3hi | 0.1 | Single | — | 87.52 | −128.03 | −0.506 ± 4.777 | 8/8 |
| TSP1 | | dose/IV | | | | | |
| CD3med | 1.0 | Single | — | 90.86 | −132.92 | 3.839 ± 1.597 | 8/8 |
| TSP1 | | dose/IV | | | | | |
| CD3med | 0.3 | Single | — | 85.13 | −124.57 | 3.214 ± 1.737 | 8/8 |
| TSP1 | | dose/IV | | | | | |
| CD3med | 0.1 | Single | — | 13.51 | −19.77 | 0.165 ± 2.561 | 8/8 |
| TSP1 | | dose/IV | | | | | |

### 8.32. Example 32: Design—Selection of Residue Positions

Design strategies were tested to produce a set of antibodies with modified Fc regions that might exhibit desired properties such as diminished effector functions. Early studies defining key amino acid binding sites on IgG for Fc gamma receptors were performed by mutational analyses, and it was determined that the lower hinge, proximal CH2 region and glycosylation of N297 were critical (Shields et al., 2001). Mutations were introduced into the regions that interact with Fc gamma receptors with the goal to diminish residual binding to Fc gamma receptors. For this particular reason, it was necessary to test various combinations of Fc positions and generate set of mutations without compromising antibody drug developability and immunogenicity risk. Several mutation sets were generated and compared to wildtype IgG1. In Examples 33-36: LALAPA-IgG1 (L234A/L235A/P329A), LALAGA-IgG1 (L234A/L235A/G237A), LALAPG-IgG1 (L234A/L235A/P329G), DAPA-IgG1 (D265A/P329A), LALASKPA-IgG1 (L234A/L235A/S267K/P329A), DAPASK-IgG1 (D265A/P329A/S267K), GADAPA-IgG1 (G237A/D265A/P329A), GADAPASK-IgG1 (G237A/D265A/P329A/S267K) and DANAPA-IgG1 (D265A/N297A/P329A) were evaluated. Previously described DAPA and DANAPA silencing motifs were included for comparison. In Examples 37-39: LALA (L234A/L235A), LALASKPA (L234A/L235A/S267K/P329A), GADAPASK (G237A/D265A/P329A/S267K) and DANAPA (D265A/N297A/P329A) mutation sets were evaluated.

### 8.33. Example 33: Expression and Purification of Fc Modified CD3 Antibodies

For the experiments described below antibodies against CD3 containing the indicated amino acid substitutions and expressed by the nucleotide sequences as indicated, were used as listed in Tables A and B. IgG1 molecules were expressed in HEK293 mammalian cells, and purified using protein A and size exclusion chromatography. In brief, heavy chain and light chain DNA of anti-CD3 WT IgG1 were synthesized at GeneArt (Regensburg, Germany) and cloned into a mammalian expression vector using restriction enzyme-ligation based cloning techniques. All variants described herein were then generated using PCR based mutagenesis. The resulting plasmids were co-transfected into HEK293T cells. For transient expression of antibodies, equal quantities of vector for each chain were co-transfected into suspension-adapted HEK293T cells using Polyethylenimine (PEI; Cat #24765 Polysciences, Inc.). Typically, 100 ml of cells in suspension at a density of 1-2 Mio cells per ml was transfected with DNA containing 50 μg of expression vector encoding the heavy chain and 50 μg expression vectors encoding the light chain. The recombinant expression vectors were then introduced into the host cells and the construct produced by further culturing of the cells for a period of 7 days to allow for secretion into the culture medium (HEK, serum-fee medium) supplemented with 0.1% pluronic acid, 4 mM glutamine, and 0.25 μg/ml antibiotic.

The produced construct was then purified from cell-free supernatant using immunoaffinity chromatography. MabSelect Sure resin (GE Healthcare Life Sciences), equilibrated with PBS buffer pH 7.4 was incubated with filtered conditioned media using liquid chromatography system (Aekta pure chromatography system, GE Healthcare Life Sciences). The resin was washed with PBS pH 7.4 before the constructs were eluted with elution buffer (50 mM citrate, 90 mM NaCl, pH 2.7). After capture, eluted proteins were pH neutralized using 1M TRIS pH 10.0 solution and polished using size exclusion chromatography technique (HiPrep Superdex 200 16/60, GE Healthcare Life Sciences). Purified proteins were finally formulated in PBS buffer pH 7.4.

### 8.34. Example 34: Biophysical Properties of Fc Modified CD3 Antibodies: SPR-Binding of Modified Antibodies to Human Fc Gamma Receptors and Human C1q

Surface plasmon resonance (SPR) experiments were performed to analyze the interaction of human activating receptors FcγR1A, FcγR3A (V158) and human C1q with IgG1 WT and antibody-Fc variants. Binding kinetics and their relative binding affinities were explored. The binding affinity is an important characteristic of an interaction between an antibody and an antigen. The equilibrium dissociation constant (KD) defines how strong the interaction is and therefore how much antibody-antigen complex is formed at equilibrium.

The knowledge of the antibody characteristics is not only essential during selection of the best therapeutic antibody candidate, but also important to understand the in vivo behavior and potentially predict cellular immune responses. The aim is to generate antibody variants with little or no binding to Fc gamma receptors to reduce or eliminate effector function aiming to improve the safety of monoclonal antibody therapeutics. Binding to human C1q was evaluated. All SPR buffers were prepared using deionized water. The samples were prepared in running buffer PBS pH 7.4 with 0.005% Tween-20. SPR measurements were measured on a Biacore T200 (GE-Healthcare Life Sciences) controlled by Biacore T200 control software version 2.0.1. Surface plasmon resonance was conducted using a Biacore T200 to assess binding affinity of antibody IgG1 WT and variants to human Fc receptors, including FcγR1A, and FcγR3A (V158) and human C1q.

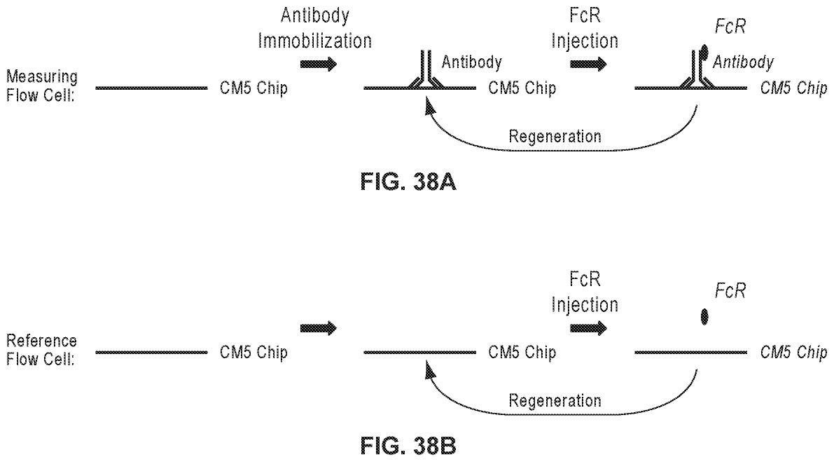

The antibodies were covalently immobilized on a CM5 sensor chip whereas Fc gamma receptors or human C1q served as analytes in solution (FIG. 38). For Fc gamma receptors binding evaluation (method 1), antibodies were diluted in 10 mM sodium acetate pH 4 and immobilized at a density of approximately 950 resonance units (RU's) on the CM5 sensor chip applying a standard amine coupling procedure. Flow cell 1 was blank immobilized to serve as a reference. The kinetic binding data were collected by subsequent injections of 1:2 dilution series of the human Fc gamma receptors on all flow cells at a flow rate of 30 μl/min and at a temperature of 25° C. The Fc gamma receptors were diluted in the running buffer at concentrations ranging from 0.2 nM to 1000 nM (e.g. FcγR1A: 0.2 to 100 nM, FcγR3A V158: 1.95 to 1000 nM). The chip surface was regenerated using 20 mM glycine pH 2.0 solution after each measuring cycle. For human C1q binding evaluation (method 2), antibodies were diluted in 10 mM sodium acetate pH 4 and immobilized at a density of approximately 7000 resonance units (RU's) on the CM5 sensor chip applying a standard amine coupling procedure. Flow cell 1 was blank immobilized to serve as a reference. The kinetic binding data were collected by subsequent injections of 1:2 dilution series of the human C1q on all flow cells at a flow rate of 30 μl/min and at a temperature of 25° C. The human C1q was diluted in the running buffer at concentrations ranging from 0.49 nM to 250 nM. The chip surface was regenerated using 50 mM NaOH solution after each measuring cycle. Zero concentration samples (blank runs) were measured for both methods to allow double-referencing during data evaluation.

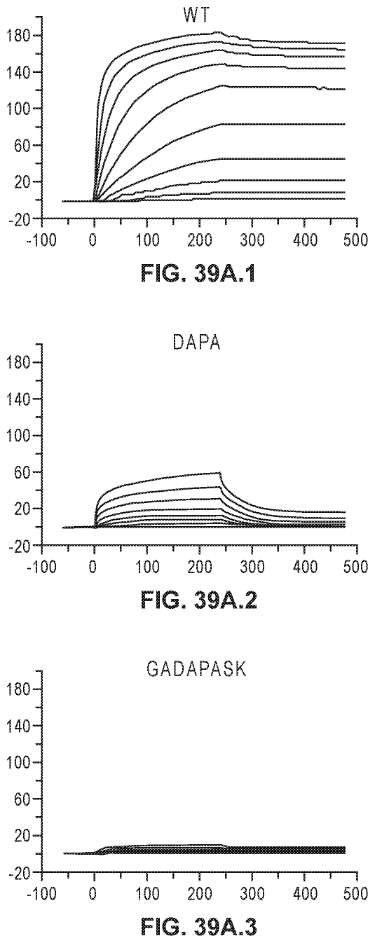

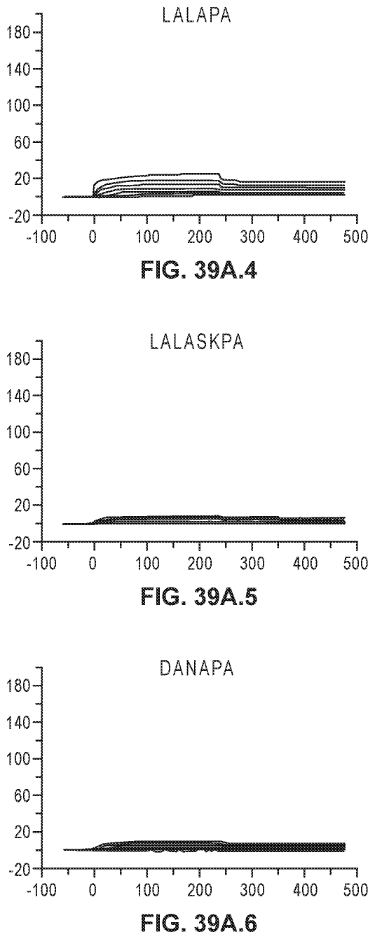

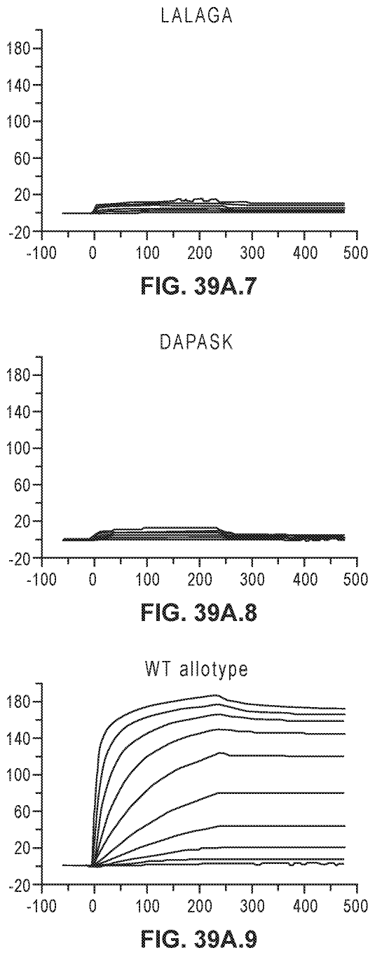

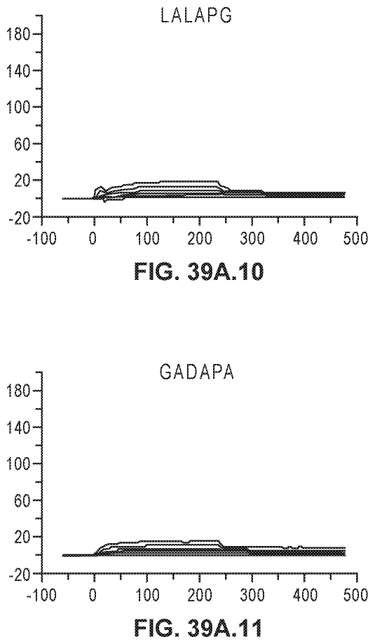

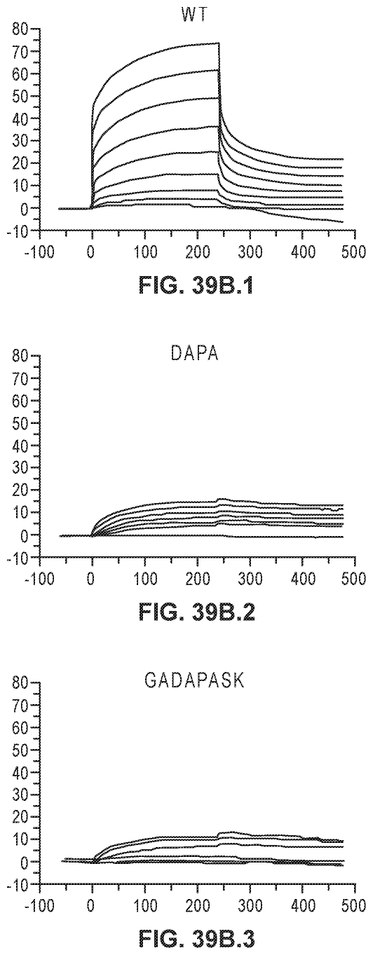

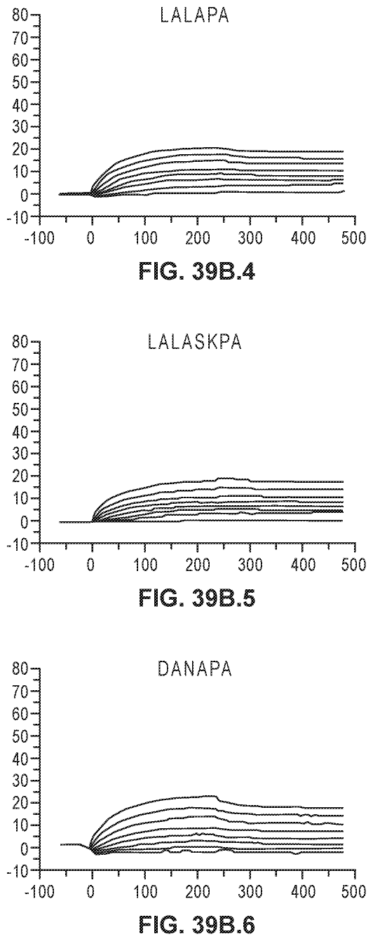

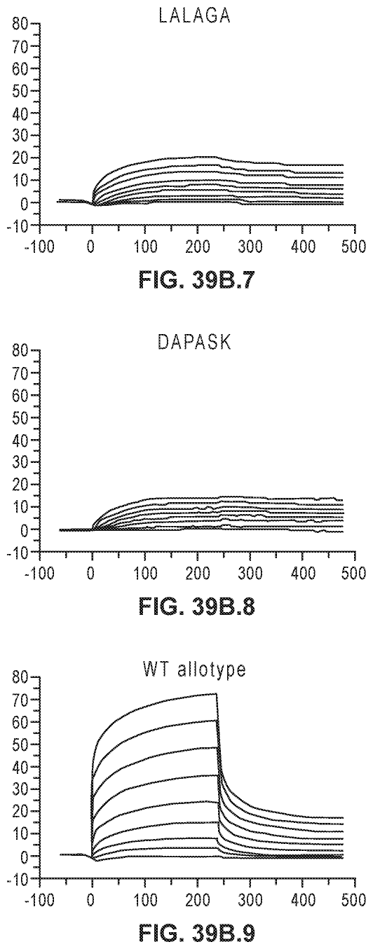

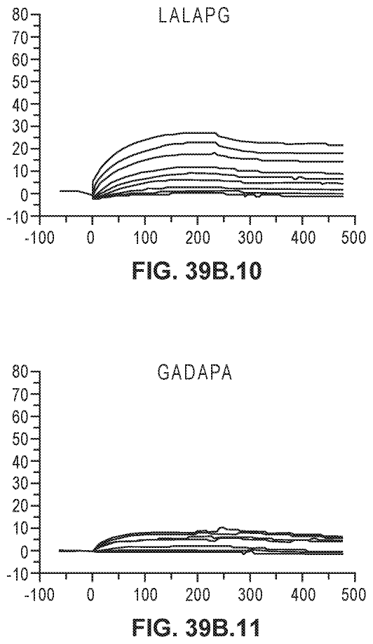

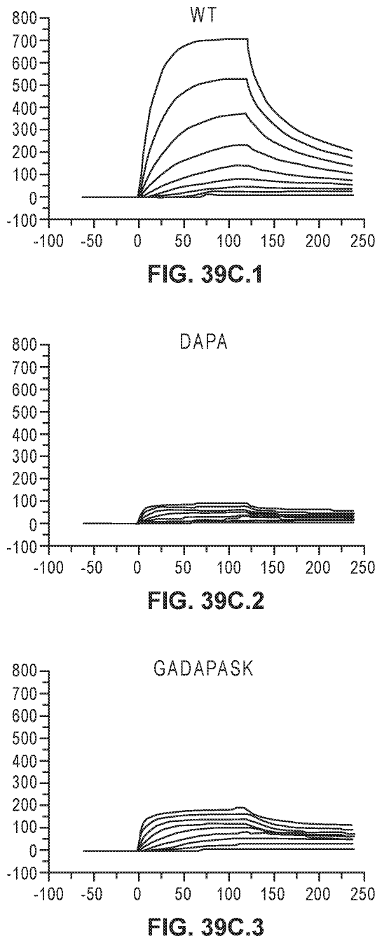

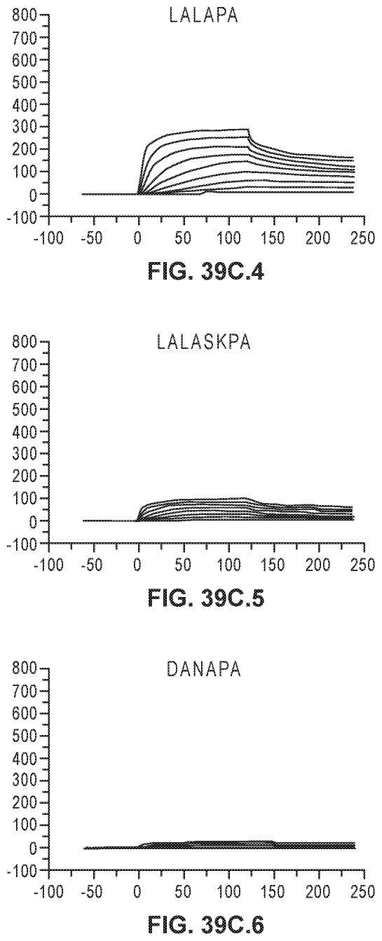

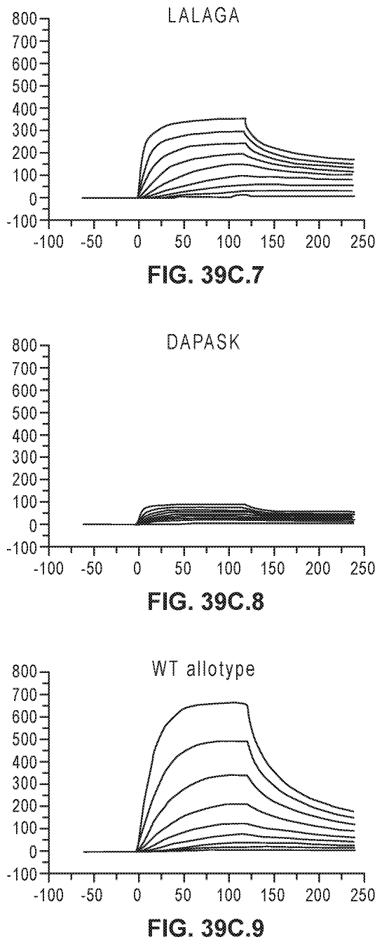

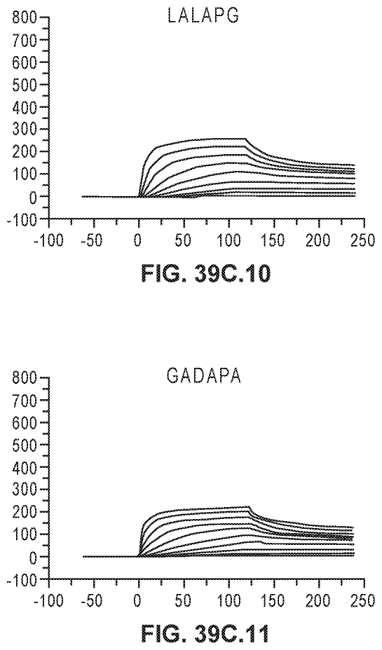

Data were evaluated using the Biacore T200 evaluation software. The raw data were double referenced, i.e. the response of the measuring flow cell was corrected for the response of the reference flow cell, and in a second step the response of a blank injection was subtracted. Then the sensorgrams were fitted by applying a 1:1 kinetic binding model to calculate dissociation equilibrium constants. In addition, the maximum response reached during the experiment was monitored. Maximum response describes the binding capacity of the surface in terms of the response at saturation. The maximum response values summarizing these interactions are given in Table 31. The SPR Biacore binding sensorgrams for each variant to each receptor were depicted in FIG. 39, Concentration range: 0.2 nM-100 nM for FcgR1, 7.8 nM-4000 nm for FcgR2A R131 d FcγR3A (V158 and F158). FIG. 39A shows representative sensorgrams and response plots of WT and variants towards FcgammaR1A (Concentration range: 0.2 nM-100 nM for human FcγR1A). FIG. 39B shows representative sensorgrams and response plots of WT and variants towards FcgammaR3A V158 (Concentration range: 1.95 nM-1000 nM for human FcγR3A V158). FIG. 39C shows representative sensorgrams and response plots of WT and variants towards human C1q (Concentration range: 0.49 nM-250 nM for human C1q). All IgG1 antibody-Fc variants inhibit the binding to Fc gamma receptors compare to WT and little or no residual binding was measured. All IgG1 antibody-Fc variants inhibit the binding to human C1q compare to WT as low residual binding was measured.

TABLE 31
Maximum responses of WT-Fc and variants towards
human FcgammaRs and C1q determined by surface
plasmon resonance

| | | Maximum | Maximum | |
|---|---|---|---|---|
| | | response at | response at | Maximum |
| | SEQ | 100 nM | 1000 nM | response |
| | ID | hFcgammaRIA | hFcgammaRIIIA | at 250 nM |
| Fc | NO | (RU) | V158 (RU) | hC1q (RU) |
| WT | | 193 | 79 | 680 |
| WT | | 193 | 72 | 640 |
| allotype | | | | |
| DANAPA | | 11 | 31 | 29 |
| DAPA | | 63 | 15 | 85 |
| LALAPA | | 26 | 19 | 270 |
| LALAPG | | 18 | 33 | 245 |
| LALAGA | | 15 | 25 | 335 |
| LALASKPA | | 9 | 20 | 95 |
| DAPASK | | 13 | 15 | 86 |
| GADAPA | | 16 | 9 | 203 |
| GADAPASK | | 10 | 12 | 168 |

### 8.35. Example 35: Differential Scanning Calorimetry- Melting Temperature of Modified Antibodies

The thermal stability of engineered antibodies CH2 domains were compared using calorimetric measurements as shown in Table 32. Calorimetric measurements were carried out on a differential scanning micro calorimeter (Nano 0SC, TA instruments). The cell volume was 0.5 ml and the heating rate was 1° C./min. All proteins were used at a concentration of 1 mg/mi in PBS (pH 7.4). The molar heat capacity of each protein was estimated by comparison with duplicate samples containing identical buffer from which the protein had been omitted. The partial molar heat capacities and melting curves were analyzed using standard procedure. Thermograms were baseline corrected and concentration normalized. The silent version LALASKPA (70LC) shows significantly better Tm compared to DANAPA (62C).

TABLE 32
Melting temperatures of WT-Fc and variants

| | Melting temperature (Tm) of |
|---|---|
| Fc | CH2 domain |
| WT | 70 |
| WT allotype R214K | 70 |
| LALAPA | 70 |
| LALAGA | 70 |
| LALAPG | 70 |
| DAPA | 65 |
| LALASKPA | 70 |
| DAPASK | 65 |
| GADAPA | 65 |
| GADAPASK | 65 |
| DANAPA | 62 |

### Aggregation Propensity Post Capture of IgG1 Anti-CD3 Antibody and Fc Variants

Size exclusion chromatography measurements were performed to evaluate the aggregation propensity (% HMW) of IgG1 antibody and Fc modified derivatives. The produced and purified anti 003 antibodies were applied to an analytical size exclusion chromatography column (SEC 200, GE Healthcare), equilibrated with PBS buffer pH 7.4. Results are summarized in Table 33.

TABLE 33
Higher molecular weight content (%) of anti-CD3
antibodies

| Fc | (%) higher molecular weight |
|---|---|
| WT | 7.3 |
| WT allotype R214K | 7.7 |
| LALAPA | 1.8 |
| LALAGA | 2.9 |
| LALAPG | 4.2 |
| DAPA | 3.9 |
| LALASKPA | 4.9 |
| DAPASK | 3.6 |
| GADAPA | 8.0 |
| GADAPASK | 6.8 |
| DANAPA | 5.9 |

### 8.36. Example 36: Anti-CD3 NFAT Signalling Assay

Jurkat reporter gene assay (RGA) for the nuclear factor of activated T-cells (N FAT) pathway was performed using Jurkat NFAT luciferized (JNL) cells and THP-1 cells (ATCC, TI1B202). THP-1 cells express FcγRI, FcγRII, and FcγRIII. Cells were co-incubated for 6 hours at 37° C., 5% C02 at a 5:1 effector to tumor ratio with each sample at the various concentrations depicted. An equal volume of ONE-Glo™ reagent (Promega, E6110) was added to the culture volume. Plate was shaken for 2 minutes, then incubated for an additional 8 minutes protected from light. For JNL+THP+IFNg experiment, THP-1 cells were pre-treated with 100 u/mL IFNg for 48 hours at 37° C., 5% C02 before co-culture. IFNg stimulation increases FcγRI expression. Luciferase activity was quantitated on the EnVision plate reader (PerkinElmer). Data was analyzed and fit to a 5 parameter-logistic curve using GraphPad Prism.

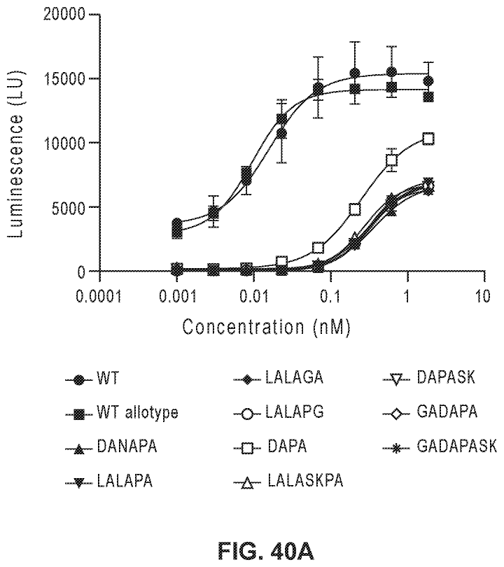

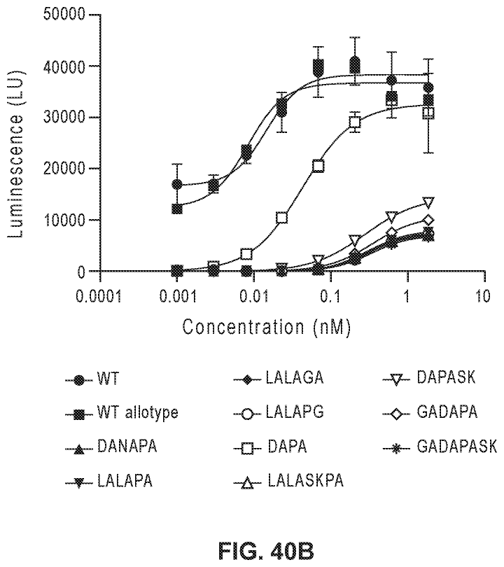

In both treatments, WT showed the greatest NFAT activity. All silencing mutation sets overall showed significantly dampened NFAT activation. In the RGA, performed without IFNg (FIG. 40A), all silencing mutation sets showed comparable T-cell activation with the exception of DAPA. When the THP-1 cells were preincubated with IFNg (FIG. 40B), the mutation sets showed lower activity, demonstrating strong Fc silencing, but some activity remaining in DAPA, LALAPA and GADAPA.

### 8.37. Example 37: Expression and Purification of Modified Antibodies

For the experiments described below antibodies were used as shown in Table 34. Designed molecules were expressed in HEK293 mammalian cells, and purified using protein A and size exclusion chromatography. In brief, heavy chains and light chain DNA were synthesized at GeneArt (Regensburg, Germany) and cloned into a mammalian expression vector using restriction enzyme-ligation based cloning techniques. The resulting plasmids were co-transfected into HEK293T cells. For transient expression of antibodies, equal quantities of vector for each chain were co-transfected into suspension-adapted HEK293T cells using Polyethylenimine (PEI; Cat #24765 Polysciences, Inc.). Typically, 100 ml of cells in suspension at a density of 1-2 Mio cells per ml was transfected with DNA containing 33 μg of expression vector encoding the first heavy chain, 33 μg of expression vector encoding the second heavy chain and 33 μg expression vectors encoding the light chain. The recombinant expression vectors were then introduced into the host cells and the construct produced by further culturing of the cells for a period of 7 days to allow for secretion into the culture medium (HEK, serum-fee medium) supplemented with 0.1% pluronic acid, 4 mM glutamine, and 0.25 μg/ml antibiotic.

The produced construct was then purified from cell-free supernatant using immunoaffinity chromatography. MabSelect Sure resin (GE Healthcare Life Sciences), equilibrated with PBS buffer pH 7.4 was incubated with filtered conditioned media using liquid chromatography system (Aekta pure chromatography system, GE Healthcare Life Sciences). The resin was washed with PBS pH 7.4 before the constructs were eluted with elution buffer (50 mM citrate, 90 mM NaCl, pH 2.7). After capture, eluted proteins were pH neutralized using 1M TRIS pH 10.0 solution and polished using size exclusion chromatography technique (HiPrep Superdex 200 16/60, GE Healthcare Life Sciences). Purified proteins were finally formulated in PBS buffer pH 7.4.

TABLE 34
Sequences of Antibodies and Fc variant

| SEQ ID | | |
|---|---|---|
| No | Description | Sequence |
| 1110 | Anti-CD19 light | eivmtqspatlsvspgeratlscrasqdvgtavawyqqkpgqaprlliywastr |
| | chain | htgiparfsgsgsgteftltisslqsedfavyfcqqyanfplytfgqgtkleikrtvaa |
| | Amino acid | psvfifppsdeqlksgtasvvcllnnfypreakvqwkvdnalqsgnsqesvteq |
| | sequence | dskdstyslsstltlskadyekhkvyacevthqglsspvtksfnrgec |
| 1111 | Anti-CD19 light | gagatcgtgatgacacagtctccagccacactgtccgtgtctccaggcgagag |
| | chain | agctacactgagctgtagagccagccaggatgtgggaacagccgtggcctg |
| | Nucleic acid | gtatcagcagaaacctggacaagcccctcggctgctgatctactgggcctcta |
| | sequence | caagacacacaggcatccctgccagattttctggcagcggctctggcaccga |
| | | gttcaccctgacaatctctagcctccagagcgaggacttcgccgtgtacttctgc |
| | | cagcagtacgccaactttcccctgtacacctttggccagggcaccaagctgga |
| | | aatcaagagaacagtggccgctccgagcgtgttcatctttccaccaagcgacg |
| | | agcagctgaaaagcggcacagcctctgtcgtgtgcctgctgaacaacttctac |
| | | cccagagaagccaaggtgcagtggaaggtggacaatgccctccagtccggc |
| | | aatagccaagagagcgtgaccgagcaggacagcaaggatagcacataca |
| | | gcctgagcagcacactgaccctgagcaaggccgactacgagaagcacaaa |
| | | gtgtacgcctgcgaagtgacacaccagggcctgtctagccctgtgaccaaga |
| | | gcttcaacagaggcgagtgc |
| 1112 | Anti-CD19-CD3 | qvqlvqsgaevkkpgasvkvsckasgytfttywiqwvrqapgqrlewmgav |
| | WT IgG1 | ypgdadtrytqkfqgrvtltadrsastaymelsslrsedtavyycgrdagleyyal |
| | Heavy Chain | dywgqgtlvtvssastkgpsvfplapsskstsggtaalgclvkdyfpepvtvsw |
| | Amino acid | nsgaltsgvhtfpavlqssglyslssvvtvpssslgtqtyicnvnhkpsntkvdkr |
| | sequence | vepkscggggsggggsevqlvesggglvqpggslklscaasgftfntyamn |
| | | wvrqasgkglewvgrirskynnyatyyadsvkdrftisrddskstlylqmnslkt |
| | | edtavyycvrhgnfgnsyvswfaywgqgtlvtvssggggsggggsggggsg |
| | | gggsqavvtqepsltvspggtvtltcrsstgavttsnyanwvqqkpgqaprglig |
| | | gtnkrapwtparfsgsllgdkaaltlsgaqpedeaeyfcalwysnlwvfgggtkl |
| | | tvlggggsdkthtcppcpapellggpsvflfppkpkdtlmisrtpevtcvvvdvs |
| | | hedpevkfnwyvdgvevhnaktkpreeqynstyrvvsvltvlhqdwlngkey |
| | | kckvsnkalpapiektiskakgqprepqvctlppsreemtknqvslscavkgfy |
| | | psdiavewesngqpennykttppvldsdgsfflvskltvdksrwqqgnvfscs |
| | | vmhealhnhytqkslslspgk |
| 1113 | Anti-CD19-CD3 | caggttcaactggttcagagcggagccgaagtgaagaaacctggggccagc |
| | WT IgG1 | gtgaaggtgtcctgtaaagccagcggctacacctttaccacctactggattcagt |
| | Heavy Chain | gggtccgacaggcccctggacagagacttgaatggatgggagctgtgtaccc |
| | Nucleic acid | cggcgacgccgataccagatacacccagaaattccagggcagagtgaccct |
| | sequence | gacagccgatagaagcgccagcacagcctacatggaactgagcagcctga |
| | | gaagcgaggataccgccgtgtactactgtggcagagatgccggcctggaata |
| | | ttacgccctggactattggggccagggcaccctggttacagtgtcctctgcctcta |
| | | caaagggcccctccgtttttccactggctcctagcagcaagagcacatctggtg |
| | | gaacagccgctctgggctgcctggtcaaggattactttcctgagcctgtgaccgt |
| | | gtcctggaatagcggagcactgacaagcggcgtgcacacatttccagccgtg |
| | | ctgcaaagcagcggcctgtactctctgtctagcgtggtcacagtgcctagctcta |
| | | gcctgggcacccagacctacatctgcaacgtgaaccacaagcctagcaaca |
| | | ccaaggtcgacaagagagtggaacccaagtcttgtggtggcggaggatctgg |
| | | cggaggcggatctgaagttcagcttgttgaatctggcggcggactggtgcaac |
| | | ctggcggatctctgaaactgtcttgtgccgcctccggcttcaccttcaatacctac |
| | | gccatgaactgggttcgacaagcctccggcaaaggactggaatgggtcgga |
| | | cggatcagaagcaagtacaacaactacgccacctactacgccgactccgtg |
| | | aaggacagattcaccatcagccgggacgactccaagagcaccctgtacctcc |
| | | agatgaactccctgaaaaccgaggacacagccgtctattattgcgtgcggcac |
| | | ggcaacttcggcaacagctatgtgtcttggtttgcctactggggacagggaacc |
| | | ctcgtgaccgtttcttcaggcggcggtggtagtggcggtggtggtagcggaggc |
| | | ggtggatcaggtggcggcggttctcaagctgtggtcacacaagagcccagcct |
| | | gacagtttctcctggcggaaccgtgacactgacctgtagatctagcaccggcg |
| | | cagtgaccaccagcaattacgctaactgggtgcagcagaagcccggccaag |
| | | ctcctagaggactgatcggaggcacaaacaagagagccccttggacaccag |
| | | ccagattttctggctctctgctgggcgataaggccgctcttacactgtctggcgca |
| | | cagcctgaagatgaggccgagtacttttgcgccctgtggtacagcaacctgtgg |
| | | gtgttcggcggaggaacaaagctgacagttcttggaggcggcggaagcgac |
| | | aagacccacacatgtcctccatgtcctgctccagaactgctcggcggaccctcc |
| | | gtgtttctgttccctccaaagccaaaggacaccctgatgatcagcagaacccct |
| | | gaagtgacctgcgtggtggtggatgtgtctcacgaggacccagaagtgaagtt |
| | | caattggtacgtggacggcgtggaagtgcacaacgccaagaccaagcctag |
| | | agaggaacagtacaacagcacctacagagtggtgtccgtgctgacagtgctg |
| | | caccaggactggctgaacggcaaagagtacaagtgcaaggtgtccaacaa |
| | | ggccctgcctgctcctatcgagaaaaccatcagcaaggccaagggccagcct |
| | | agggaacctcaagtgtgcactctgcctccaagccgggaagagatgaccaag |
| | | aatcaggtgtccctgagctgcgccgtgaagggcttttacccttccgatatcgccg |
| | | tggaatgggagagcaatggccagccagagaacaactacaagaccacacct |
| | | cctgtgctggacagcgacggctcattcttcctggtgtctaagctgaccgtggaca |
| | | agagcagatggcagcagggcaacgtgttcagctgttctgtgatgcacgaggc |
| | | cctgcacaaccactacacacagaagtccctgtctctgagccccggcaaa |
| 1114 | Anti-CD58 | sqqiygvvygnvtfhvpsnvplkevlwkkqkdkvaelensefrafssfknrvyl |
| | WT IgG1 | dtvsgsltiynltssdedeyemespnitdtmkfflyvlesggggsdkthtcppcp |
| | Heavy Chain | apellggpsvflfppkpkdtlmisrtpevtcvvvdvshedpevkfnwyvdgvev |
| | Amino acid | hnaktkpreeqynstyrvvsvltvlhqdwlngkeykckvsnkalpapiektisk |
| | sequence | akgqprepqvytlppcreemtknqvslwclvkgfypsdiavewesngqpen |
| | | nykttppvldsdgsfflyskltvdksrwqqgnvfscsvmhealhnhytqkslsls |
| | | pgk |
| 1115 | Anti-CD58 | tctcagcagatctatggcgtggtgtacggcaacgtgaccttccacgtgccaagc |
| | WT IgG1 | aacgtgcccctgaaagaggtgctctggaagaaacagaaagacaaggtggc |
| | Heavy Chain | cgagctggaaaacagcgagttcagagccttcagcagcttcaagaaccgggt |
| | Nucleic acid | gtacctggataccgtgtccggcagcctgaccatctacaatctgaccagcagcg |
| | sequence | acgaggacgagtacgagatggaaagccccaacatcaccgacaccatgaag |
| | | ttctttctgtacgtgctggaaagcggcggaggcggctctgataagacacacac |
| | | atgtcctccatgtcctgctccagagctgctcggcggaccttctgttttcctgtttcca |
| | | cctaagccaaaggacaccctgatgatcagcagaacccctgaagtgacctgc |
| | | gtggtggtggatgtgtctcacgaggaccccgaagtgaagttcaattggtacgtg |
| | | gacggcgtggaagtgcacaacgccaagaccaagcctagagaggaacagt |
| | | acaacagcacctacagagtggtgtccgtgctgaccgtgctgcaccaggattgg |
| | | ctgaacggcaaagagtacaagtgcaaggtgtccaacaaggccctgcctgctc |
| | | ctatcgagaaaaccatcagcaaggccaagggccagccaagagaacccca |
| | | ggtttacaccctgcctccatgccgggaagagatgaccaagaatcaggtgtccc |
| | | tgtggtgcctggtcaagggcttctacccttccgatatcgccgtggaatgggaga |
| | | gcaatggccagcctgagaacaactacaagaccacacctcctgtgctggaca |
| | | gcgacggctcattcttcctgtacagcaagctgacagtggacaagagcagatg |
| | | gcagcagggcaacgtgttcagctgttctgtgatgcacgaggccctgcacaacc |
| | | actacacccagaagtctctgtctctgagccccggcaaa |
| 1116 | Anti-CD19-CD3 | qvqlvqsgaevkkpgasvkvsckasgytfttywiqwvrqapgqrlewmgav |
| | DANAPA IgG1 | ypgdadtrytqkfqgrvtltadrsastaymelsslrsedtavyycgrdagleyyal |
| | Heavy Chain | dywgqgtlvtvssastkgpsvfplapsskstsggtaalgclvkdyfpepvtvsw |
| | Amino acid | nsgaltsgvhtfpavlqssglyslssvvtvpssslgtqtyicnvnhkpsntkvdkr |
| | sequence | vepkscggggsggggsevqlvesggglvqpggslklscaasgftfntyamn |
| | | wvrqasgkglewvgrirskynnyatyyadsvkdrftisrddskstlylqmnslkt |
| | | edtavyycvrhgnfgnsyvswfaywgqgtlvtvssggggsggggsggggsg |
| | | gggsqavvtqepsltvspggtvtltcrsstgavttsnyanwvqqkpgqaprglig |
| | | gtnkrapwtparfsgsllgdkaaltlsgaqpedeaeyfcalwysnlwvfgggtkl |
| | | tvlggggsdkthtcppcpapellggpsvflfppkpkdtlmisrtpevtcvvvavs |
| | | hedpevkfnwyvdgvevhnaktkpreeqyastyrvvsvltvlhqdwlngkey |
| | | kckvsnkalaapiektiskakgqprepqvctlppsreemtknqvslscavkgfy |
| | | psdiavewesngqpennykttppvldsdgsfflvskltvdksrwqqgnvfscs |
| | | vmhealhnhytqkslslspgk |
| 1117 | Anti-CD19-CD3 | caggttcaactggttcagagcggagccgaagtgaagaaacctggggccagc |
| | DANAPA IgG1 | gtgaaggtgtcctgtaaagccagcggctacacctttaccacctactggattcagt |
| | Heavy Chain | gggtccgacaggcccctggacagagacttgaatggatgggagctgtgtaccc |
| | Nucleic acid | cggcgacgccgataccagatacacccagaaattccagggcagagtgaccct |
| | sequence | gacagccgatagaagcgccagcacagcctacatggaactgagcagcctga |
| | | gaagcgaggataccgccgtgtactactgtggcagagatgccggcctggaata |
| | | ttacgccctggactattggggccagggcaccctggttacagtgtcctctgcctcta |
| | | caaagggcccctccgtttttccactggctcctagcagcaagagcacatctggtg |
| | | gaacagccgctctgggctgcctggtcaaggattactttcctgagcctgtgaccgt |
| | | gtcctggaatagcggagcactgacaagcggcgtgcacacatttccagccgtg |
| | | ctgcaaagcagcggcctgtactctctgtctagcgtggtcacagtgcctagctcta |
| | | gcctgggcacccagacctacatctgcaacgtgaaccacaagcctagcaaca |
| | | ccaaggtcgacaagagagtggaacccaagtcttgtggtggcggaggatctgg |
| | | cggaggcggatctgaagttcagcttgttgaatctggcggcggactggtgcaac |
| | | ctggcggatctctgaaactgtcttgtgccgcctccggcttcaccttcaatacctac |
| | | gccatgaactgggttcgacaagcctccggcaaaggactggaatgggtcgga |
| | | cggatcagaagcaagtacaacaactacgccacctactacgccgactccgtg |
| | | aaggacagattcaccatcagccgggacgactccaagagcaccctgtacctcc |
| | | agatgaactccctgaaaaccgaggacacagccgtctattattgcgtgcggcac |
| | | ggcaacttcggcaacagctatgtgtcttggtttgcctactggggacagggaacc |
| | | ctcgtgaccgtttcttcaggcggcggtggtagtggcggtggtggtagcggaggc |
| | | ggtggatcaggtggcggcggttctcaagctgtggtcacacaagagcccagcct |
| | | gacagtttctcctggcggaaccgtgacactgacctgtagatctagcaccggcg |
| | | cagtgaccaccagcaattacgctaactgggtgcagcagaagcccggccaag |
| | | ctcctagaggactgatcggaggcacaaacaagagagccccttggacaccag |
| | | ccagattttctggctctctgctgggcgataaggccgctcttacactgtctggcgca |
| | | cagcctgaagatgaggccgagtacttttgcgccctgtggtacagcaacctgtgg |
| | | gtgttcggcggaggaacaaagctgacagttcttggaggcggcggaagcgac |
| | | aagacccacacatgtcctccatgtcctgctccagaactgctcggcggaccctcc |
| | | gtgtttctgttccctccaaagccaaaggacaccctgatgatcagcagaacccct |
| | | gaagtgacctgtgtggtggtggccgtgtctcacgaggacccagaagtgaagtt |
| | | caattggtacgtggacggcgtggaagtgcacaacgccaagaccaagcctag |
| | | agaggaacagtacgccagcacctacagagtggtgtctgtgctgacagtgctg |
| | | caccaggactggctgaacggcaaagagtacaagtgcaaggtgtccaacaa |
| | | ggccctggccgctcctatcgagaaaaccatctctaaggccaagggccagcct |
| | | cgggaacctcaagtgtgcactcttccacctagccgggaagagatgaccaaga |
| | | accaggtgtcactgagctgcgccgtgaagggcttctacccttctgatatcgccgt |
| | | ggaatgggagagcaacggccagccagagaacaactacaagaccacacct |
| | | cctgtgctggacagcgacggctcattcttcctggtgtctaagctgaccgtggaca |
| | | agagcagatggcagcagggcaacgtgttcagctgttctgtgatgcacgaggc |
| | | cctgcacaaccactacacacagaagtccctgtctctgagccccggcaaa |
| 1118 | Anti-CD58 | sqqiygvvygnvtfhvpsnvplkevlwkkqkdkvaelensefrafssfknrvyl |
| | DANAPA IgG1 | dtvsgsltiynltssdedeyemespnitdtmkfflyvlesggggsdkthtcppcp |
| | Heavy Chain | apellggpsvflfppkpkdtlmisrtpevtcvvvavshedpevkfnwyvdgvev |
| | Amino acid | hnaktkpreeqyastyrvvsvltvlhqdwlngkeykckvsnkalaapiektisk |
| | sequence | akgqprepqvytlppcreemtknqvslwclvkgfypsdiavewesngqpen |
| | | nykttppvldsdgsfflyskltvdksrwqqgnvfscsvmhealhnhytqkslsls |
| | | pgk |
| 1119 | Anti-CD58 | tctcagcagatctatggcgtggtgtacggcaacgtgaccttccacgtgccaagc |
| | DANAPA IgG1 | aacgtgcccctgaaagaggtgctctggaagaaacagaaagacaaggtggc |
| | Heavy Chain | cgagctggaaaacagcgagttcagagccttcagcagcttcaagaaccgggt |
| | Nucleic acid | gtacctggataccgtgtccggcagcctgaccatctacaatctgaccagcagcg |
| | sequence | acgaggacgagtacgagatggaaagccccaacatcaccgacaccatgaag |
| | | ttctttctgtacgtgctggaaagcggcggaggcggctctgataagacacacac |
| | | atgtcctccatgtcctgctccagagctgctcggcggaccttctgttttcctgtttcca |
| | | cctaagccaaaggacaccctgatgatcagcagaacccctgaagtgacctgtg |
| | | tggtggtggccgtgtctcacgaagatcccgaagtgaagttcaattggtacgtgg |
| | | acggcgtggaagtgcacaacgccaagaccaagcctagagaggaacagta |
| | | cgccagcacctatagagtggtgtccgtgctgacagtgctgcaccaggactggc |
| | | tgaacggcaaagagtacaagtgcaaggtgtccaacaaggccctggccgctc |
| | | ctatcgagaaaaccatctctaaggccaagggccagcctcgggaaccacagg |
| | | tttacaccctgcctccatgccgggaagagatgaccaagaatcaggtgtccctgt |
| | | ggtgcctggtcaagggcttctacccttccgatatcgccgtggaatgggagagca |
| | | atggccagcctgagaacaactacaagaccacacctcctgtgctggacagcg |
| | | acggctcattcttcctgtacagcaagctgaccgtggacaagagcagatggcag |
| | | cagggcaacgtgttcagctgttctgtgatgcacgaggccctgcacaaccacta |
| | | cacccagaagtctctgtctctgagccccggcaaa |
| 1120 | Anti-CD19-CD3 | qvqlvqsgaevkkpgasvkvsckasgytfttywiqwvrqapgqrlewmgav |
| | GADAPASK IgG1 | ypgdadtrytqkfqgrvtltadrsastaymelsslrsedtavyycgrdagleyyal |
| | Heavy Chain | dywgqgtlvtvssastkgpsvfplapsskstsggtaalgclvkdyfpepvtvsw |
| | Amino acid | nsgaltsgvhtfpavlqssglyslssvvtvpssslgtqtyicnvnhkpsntkvdkr |
| | sequence | vepkscggggsggggsevqlvesggglvqpggslklscaasgftfntyamn |
| | | wvrqasgkglewvgrirskynnyatyyadsvkdrftisrddskstlylqmnslkt |
| | | edtavyycvrhgnfgnsyvswfaywgqgtlvtvssggggsggggsggggsg |
| | | gggsqavvtqepsltvspggtvtltcrsstgavttsnyanwvqqkpgqaprglig |
| | | gtnkrapwtparfsgsllgdkaaltlsgaqpedeaeyfcalwysnlwvfgggtkl |
| | | tvlggggsdkthtcppcpapellgapsvflfppkpkdtlmisrtpevtcvvvavk |
| | | hedpevkfnwyvdgvevhnaktkpreeqynstyrvvsvltvlhqdwlngkey |
| | | kckvsnkalaapiektiskakgqprepqvctlppsreemtknqvslscavkgfy |
| | | psdiavewesngqpennykttppvldsdgsfflvskltvdksrwqqgnvfscs |
| | | vmhealhnhytqkslslspgk |
| 1121 | Anti-CD19-CD3 | caggttcaactggttcagagcggagccgaagtgaagaaacctggggccagc |
| | GADAPASK IgG1 | gtgaaggtgtcctgtaaagccagcggctacacctttaccacctactggattcagt |
| | Heavy Chain | gggtccgacaggcccctggacagagacttgaatggatgggagctgtgtaccc |
| | Nucleic acid | cggcgacgccgataccagatacacccagaaattccagggcagagtgaccct |
| | sequence | gacagccgatagaagcgccagcacagcctacatggaactgagcagcctga |
| | | gaagcgaggataccgccgtgtactactgtggcagagatgccggcctggaata |
| | | ttacgccctggactattggggccagggcaccctggttacagtgtcctctgcctcta |
| | | caaagggcccctccgtttttccactggctcctagcagcaagagcacatctggtg |
| | | gaacagccgctctgggctgcctggtcaaggattactttcctgagcctgtgaccgt |
| | | gtcctggaatagcggagcactgacaagcggcgtgcacacatttccagccgtg |
| | | ctgcaaagcagcggcctgtactctctgtctagcgtggtcacagtgcctagctcta |
| | | gcctgggcacccagacctacatctgcaacgtgaaccacaagcctagcaaca |
| | | ccaaggtcgacaagagagtggaacccaagtcttgtggtggcggaggatctgg |
| | | cggaggcggatctgaagttcagcttgttgaatctggcggcggactggtgcaac |
| | | ctggcggatctctgaaactgtcttgtgccgcctccggcttcaccttcaatacctac |
| | | gccatgaactgggttcgacaagcctccggcaaaggactggaatgggtcgga |
| | | cggatcagaagcaagtacaacaactacgccacctactacgccgactccgtg |
| | | aaggacagattcaccatcagccgggacgactccaagagcaccctgtacctcc |
| | | agatgaactccctgaaaaccgaggacacagccgtctattattgcgtgcggcac |
| | | ggcaacttcggcaacagctatgtgtcttggtttgcctactggggacagggaacc |
| | | ctcgtgaccgtttcttcaggcggcggtggtagtggcggtggtggtagcggaggc |
| | | ggtggatcaggtggcggcggttctcaagctgtggtcacacaagagcccagcct |
| | | gacagtttctcctggcggaaccgtgacactgacctgtagatctagcaccggcg |
| | | cagtgaccaccagcaattacgctaactgggtgcagcagaagcccggccaag |
| | | ctcctagaggactgatcggaggcacaaacaagagagccccttggacaccag |
| | | ccagattttctggctctctgctgggcgataaggccgctcttacactgtctggcgca |
| | | cagcctgaagatgaggccgagtacttttgcgccctgtggtacagcaacctgtgg |
| | | gtgttcggcggaggaacaaagctgacagttcttggaggcggcggaagcgac |
| | | aagacccacacatgtcctccatgtcctgctccagaactgctgggagcccctag |
| | | cgttttcctgtttcctccaaagccaaaggacaccctgatgatcagcagaacccct |
| | | gaagtgacctgtgtggtggtggccgtgaagcacgaggacccagaagtgaagt |
| | | tcaattggtacgtggacggcgtggaagtgcacaacgccaagaccaagccta |
| | | gagaggaacagtacaacagcacctacagagtggtgtccgtgctgacagtgct |
| | | gcaccaggactggctgaacggcaaagagtacaagtgcaaggtgtccaaca |
| | | aggccctggccgctcctatcgagaaaaccatctctaaggccaagggccagcc |
| | | tcgggaacctcaagtgtgcactcttccacctagccgggaagagatgaccaag |
| | | aaccaggtgtcactgagctgcgccgtgaagggcttctacccttctgatatcgcc |
| | | gtggaatgggagagcaacggccagccagagaacaactacaagaccacac |
| | | ctcctgtgctggacagcgacggctcattcttcctggtgtctaagctgaccgtgga |
| | | caagagcagatggcagcagggcaacgtgttcagctgttctgtgatgcacgag |
| | | gccctgcacaaccactacacacagaagtccctgtctctgagccccggcaaa |
| 1122 | Anti-CD58 | sqqiygvvygnvtfhvpsnvplkevlwkkqkdkvaelensefrafssfknrvyl |
| | GADAPASK IgG1 | dtvsgsltiynltssdedeyemespnitdtmkfflyvlesggggsdkthtcppcp |
| | Heavy Chain | apellgapsvflfppkpkdtlmisrtpevtcvvvavkhedpevkfnwyvdgvev |
| | Amino acid | hnaktkpreeqynstyrvvsvltvlhqdwlngkeykckvsnkalaapiektisk |
| | sequence | akgqprepqvytlppcreemtknqvslwclvkgfypsdiavewesngqpen |
| | | nykttppvldsdgsfflyskltvdksrwqqgnvfscsvmhealhnhytqkslsls |
| | | pgk |
| 1123 | Anti-CD58 | tctcagcagatctatggcgtggtgtacggcaacgtgaccttccacgtgccaagc |
| | GADAPASK IgG1 | aacgtgcccctgaaagaggtgctctggaagaaacagaaagacaaggtggc |
| | Heavy Chain | cgagctggaaaacagcgagttcagagccttcagcagcttcaagaaccgggt |
| | Nucleic acid | gtacctggataccgtgtccggcagcctgaccatctacaatctgaccagcagcg |
| | sequence | acgaggacgagtacgagatggaaagccccaacatcaccgacaccatgaag |
| | | ttctttctgtacgtgctggaaagcggcggaggcggctctgataagacacacac |
| | | atgtcctccatgtcctgctccagaactgctgggagcccctagcgttttcctgtttcct |
| | | ccaaagccaaaggacaccctgatgatcagcagaacccctgaagtgacctgt |
| | | gtggtggtggccgtgaagcacgaagatcccgaagtgaagttcaattggtacgt |
| | | ggacggcgtggaagtgcacaacgccaagaccaagcctagagaggaacag |
| | | tacaacagcacctacagagtggtgtccgtgctgaccgtgctgcaccaggattg |
| | | gctgaacggcaaagagtacaagtgcaaggtgtccaacaaggccctggccgc |
| | | tcctatcgagaaaaccatctctaaggccaagggccagcctcgggaaccaca |
| | | ggtttacaccctgcctccatgccgggaagagatgaccaagaatcaggtgtccc |
| | | tgtggtgcctggtcaagggcttctacccttccgatatcgccgtggaatgggaga |
| | | gcaatggccagcctgagaacaactacaagaccacacctcctgtgctggaca |
| | | gcgacggctcattcttcctgtacagcaagctgacagtggacaagagcagatg |
| | | gcagcagggcaacgtgttcagctgttctgtgatgcacgaggccctgcacaacc |
| | | actacacccagaagtctctgtctctgagccccggcaaa |
| 1124 | Anti-CD19-CD3 | qvqlvqsgaevkkpgasvkvsckasgytfttywiqwvrqapgqrlewmgav |
| | LALA IgG1 | ypgdadtrytqkfqgrvtltadrsastaymelsslrsedtavyycgrdagleyyal |
| | Heavy Chain | dywgqgtlvtvssastkgpsvfplapsskstsggtaalgclvkdyfpepvtvsw |
| | Amino acid | nsgaltsgvhtfpavlqssglyslssvvtvpssslgtqtyicnvnhkpsntkvdkr |
| | sequence | vepkscggggsggggsevqlvesggglvqpggslklscaasgftfntyamn |
| | | wvrqasgkglewvgrirskynnyatyyadsvkdrftisrddskstlylqmnslkt |
| | | edtavyycvrhgnfgnsyvswfaywgqgtlvtvssggggsggggsggggsg |
| | | gggsqavvtqepsltvspggtvtltcrsstgavttsnyanwvqqkpgqaprglig |
| | | gtnkrapwtparfsgsllgdkaaltlsgaqpedeaeyfcalwysnlwvfgggtkl |
| | | tvlggggsdkthtcppcpapeaaggpsvflfppkpkdtlmisrtpevtcvvvdv |
| | | shedpevkfnwyvdgvevhnaktkpreeqynstyrvvsvltvlhqdwlngke |
| | | ykckvsnkalpapiektiskakgqprepqvctlppsreemtknqvslscavkgf |
| | | ypsdiavewesngqpennykttppvldsdgsfflvskltvdksrwqqgnvfsc |
| | | svmhealhnhytqkslslspgk |
| 1125 | Anti-CD19-CD3 | caggttcaactggttcagagcggagccgaagtgaagaaacctggggccagc |
| | LALA IgG1 | gtgaaggtgtcctgtaaagccagcggctacacctttaccacctactggattcagt |
| | Heavy Chain | gggtccgacaggcccctggacagagacttgaatggatgggagctgtgtaccc |
| | Nucleic acid | cggcgacgccgataccagatacacccagaaattccagggcagagtgaccct |
| | sequence | gacagccgatagaagcgccagcacagcctacatggaactgagcagcctga |
| | | gaagcgaggataccgccgtgtactactgtggcagagatgccggcctggaata |
| | | ttacgccctggactattggggccagggcaccctggttacagtgtcctctgcctcta |
| | | caaagggcccctccgtttttccactggctcctagcagcaagagcacatctggtg |
| | | gaacagccgctctgggctgcctggtcaaggattactttcctgagcctgtgaccgt |
| | | gtcctggaatagcggagcactgacaagcggcgtgcacacatttccagccgtg |
| | | ctgcaaagcagcggcctgtactctctgtctagcgtggtcacagtgcctagctcta |
| | | gcctgggcacccagacctacatctgcaacgtgaaccacaagcctagcaaca |
| | | ccaaggtcgacaagagagtggaacccaagtcttgtggtggcggaggatctgg |
| | | cggaggcggatctgaagttcagcttgttgaatctggcggcggactggtgcaac |
| | | ctggcggatctctgaaactgtcttgtgccgcctccggcttcaccttcaatacctac |
| | | gccatgaactgggttcgacaagcctccggcaaaggactggaatgggtcgga |
| | | cggatcagaagcaagtacaacaactacgccacctactacgccgactccgtg |
| | | aaggacagattcaccatcagccgggacgactccaagagcaccctgtacctcc |
| | | agatgaactccctgaaaaccgaggacacagccgtctattattgcgtgcggcac |
| | | ggcaacttcggcaacagctatgtgtcttggtttgcctactggggacagggaacc |
| | | ctcgtgaccgtttcttcaggcggcggtggtagtggcggtggtggtagcggaggc |
| | | ggtggatcaggtggcggcggttctcaagctgtggtcacacaagagcccagcct |
| | | gacagtttctcctggcggaaccgtgacactgacctgtagatctagcaccggcg |
| | | cagtgaccaccagcaattacgctaactgggtgcagcagaagcccggccaag |
| | | ctcctagaggactgatcggaggcacaaacaagagagccccttggacaccag |
| | | ccagattttctggctctctgctgggcgataaggccgctcttacactgtctggcgca |
| | | cagcctgaagatgaggccgagtacttttgcgccctgtggtacagcaacctgtgg |
| | | gtgttcggcggaggaacaaagctgacagttcttggaggcggcggaagcgac |
| | | aagacccacacatgtcctccatgtcctgctccagaagctgctggcggaccctc |
| | | cgtgtttctgttccctccaaagccaaaggacaccctgatgatcagcagaacccc |
| | | tgaagtgacctgcgtggtggtggatgtgtctcacgaggacccagaagtgaagtt |
| | | caattggtacgtggacggcgtggaagtgcacaacgccaagaccaagcctag |
| | | agaggaacagtacaacagcacctacagagtggtgtccgtgctgacagtgctg |
| | | caccaggactggctgaacggcaaagagtacaagtgcaaggtgtccaacaa |
| | | ggccctgcctgctcctatcgagaaaaccatcagcaaggccaagggccagcct |
| | | agggaacctcaagtgtgcactctgcctccaagccgggaagagatgaccaag |
| | | aatcaggtgtccctgagctgcgccgtgaagggcttttacccttccgatatcgccg |
| | | tggaatgggagagcaatggccagccagagaacaactacaagaccacacct |
| | | cctgtgctggacagcgacggctcattcttcctggtgtctaagctgaccgtggaca |
| | | agagcagatggcagcagggcaacgtgttcagctgttctgtgatgcacgaggc |
| | | cctgcacaaccactacacacagaagtccctgtctctgagccccggcaaa |
| 1126 | Anti-CD58 | sqqiygvvygnvtfhvpsnvplkevlwkkqkdkvaelensefrafssfknrvyl |
| | LALA IgG1 | dtvsgsltiynltssdedeyemespnitdtmkfflyvlesggggsdkthtcppcp |
| | Heavy Chain | apeaaggpsvflfppkpkdtlmisrtpevtcvvvdvshedpevkfnwyvdgv |
| | Amino acid | evhnaktkpreeqynstyrvvsvltvlhqdwlngkeykckvsnkalpapiekti |
| | sequence | skakgqprepqvytlppcreemtknqvslwclvkgfypsdiavewesngqp |
| | | ennykttppvldsdgsfflyskltvdksrwqqgnvfscsvmhealhnhytqksl |
| | | slspgk |
| 1314 | Anti-CD58 | tctcagcagatctatggcgtggtgtacggcaacgtgaccttccacgtgccaagc |
| | LALA IgG1 | aacgtgcccctgaaagaggtgctctggaagaaacagaaagacaaggtggc |
| | Heavy Chain | cgagctggaaaacagcgagttcagagccttcagcagcttcaagaaccgggt |
| | Nucleic acid | gtacctggataccgtgtccggcagcctgaccatctacaatctgaccagcagcg |
| | sequence | acgaggacgagtacgagatggaaagccccaacatcaccgacaccatgaag |
| | | ttctttctgtacgtgctggaaagcggcggaggcggctctgataagacacacac |
| | | atgtcctccatgtcctgctccagaagcagctggcggaccctctgttttcctgtttcc |
| | | acctaagccaaaggacaccctgatgatcagcagaacccctgaagtgacctg |
| | | cgtggtggtggatgtgtctcacgaggaccccgaagtgaagttcaattggtacgt |
| | | ggacggcgtggaagtgcacaacgccaagaccaagcctagagaggaacag |
| | | tacaacagcacctacagagtggtgtccgtgctgaccgtgctgcaccaggattg |
| | | gctgaacggcaaagagtacaagtgcaaggtgtccaacaaggccctgcctgct |
| | | cctatcgagaaaaccatcagcaaggccaagggccagccaagagaacccc |
| | | aggtttacaccctgcctccatgccgggaagagatgaccaagaatcaggtgtcc |
| | | ctgtggtgcctggtcaagggcttctacccttccgatatcgccgtggaatgggaga |
| | | gcaatggccagcctgagaacaactacaagaccacacctcctgtgctggaca |
| | | gcgacggctcattcttcctgtacagcaagctgacagtggacaagagcagatg |
| | | gcagcagggcaacgtgttcagctgttctgtgatgcacgaggccctgcacaacc |
| | | actacacccagaagtctctgtctctgagccccggcaaa |
| 1127 | Anti-CD19-CD3 | qvqlvqsgaevkkpgasvkvsckasgytfttywiqwvrqapgqrlewmgav |
| | LALASKPA IgG1 | ypgdadtrytqkfqgrvtltadrsastaymelsslrsedtavyycgrdagleyyal |
| | Heavy Chain | dywgqgtlvtvssastkgpsvfplapsskstsggtaalgclvkdyfpepvtvsw |
| | Amino acid | nsgaltsgvhtfpavlqssglyslssvvtvpssslgtqtyicnvnhkpsntkvdkr |
| | sequence | vepkscggggsggggsevqlvesggglvqpggslklscaasgftfntyamn |
| | | wvrqasgkglewvgrirskynnyatyyadsvkdrftisrddskstlylqmnslkt |
| | | edtavyycvrhgnfgnsyvswfaywgqgtlvtvssggggsggggsggggsg |
| | | gggsqavvtqepsltvspggtvtltcrsstgavttsnyanwvqqkpgqaprglig |
| | | gtnkrapwtparfsgsllgdkaaltlsgaqpedeaeyfcalwysnlwvfgggtkl |
| | | tvlggggsdkthtcppcpapeaaggpsvflfppkpkdtlmisrtpevtcvvvdv |
| | | khedpevkfnwyvdgvevhnaktkpreeqynstyrvvsvltvlhqdwlngke |
| | | ykckvsnkalaapiektiskakgqprepqvctlppsreemtknqvslscavkgf |
| | | ypsdiavewesngqpennykttppvldsdgsfflvskltvdksrwqqgnvfsc |
| | | svmhealhnhytqkslslspgk |
| 1128 | Anti-CD19-CD3 | caggttcaactggttcagagcggagccgaagtgaagaaacctggggccagc |
| | LALASKPA IgG1 | gtgaaggtgtcctgtaaagccagcggctacacctttaccacctactggattcagt |
| | Heavy Chain | gggtccgacaggcccctggacagagacttgaatggatgggagctgtgtaccc |
| | Nucleic acid | cggcgacgccgataccagatacacccagaaattccagggcagagtgaccct |
| | sequence | gacagccgatagaagcgccagcacagcctacatggaactgagcagcctga |
| | | gaagcgaggataccgccgtgtactactgtggcagagatgccggcctggaata |
| | | ttacgccctggactattggggccagggcaccctggttacagtgtcctctgcctcta |
| | | caaagggcccctccgtttttccactggctcctagcagcaagagcacatctggtg |
| | | gaacagccgctctgggctgcctggtcaaggattactttcctgagcctgtgaccgt |
| | | gtcctggaatagcggagcactgacaagcggcgtgcacacatttccagccgtg |
| | | ctgcaaagcagcggcctgtactctctgtctagcgtggtcacagtgcctagctcta |
| | | gcctgggcacccagacctacatctgcaacgtgaaccacaagcctagcaaca |
| | | ccaaggtcgacaagagagtggaacccaagtcttgtggtggcggaggatctgg |
| | | cggaggcggatctgaagttcagcttgttgaatctggcggcggactggtgcaac |
| | | ctggcggatctctgaaactgtcttgtgccgcctccggcttcaccttcaatacctac |
| | | gccatgaactgggttcgacaagcctccggcaaaggactggaatgggtcgga |
| | | cggatcagaagcaagtacaacaactacgccacctactacgccgactccgtg |
| | | aaggacagattcaccatcagccgggacgactccaagagcaccctgtacctcc |
| | | agatgaactccctgaaaaccgaggacacagccgtctattattgcgtgcggcac |
| | | ggcaacttcggcaacagctatgtgtcttggtttgcctactggggacagggaacc |
| | | ctcgtgaccgtttcttcaggcggcggtggtagtggcggtggtggtagcggaggc |
| | | ggtggatcaggtggcggcggttctcaagctgtggtcacacaagagcccagcct |
| | | gacagtttctcctggcggaaccgtgacactgacctgtagatctagcaccggcg |
| | | cagtgaccaccagcaattacgctaactgggtgcagcagaagcccggccaag |
| | | ctcctagaggactgatcggaggcacaaacaagagagccccttggacaccag |
| | | ccagattttctggctctctgctgggcgataaggccgctcttacactgtctggcgca |
| | | cagcctgaagatgaggccgagtacttttgcgccctgtggtacagcaacctgtgg |
| | | gtgttcggcggaggaacaaagctgacagttcttggaggcggcggaagcgac |
| | | aagacccacacatgtcctccatgtcctgctccagaagctgctggcggaccctc |
| | | cgtgtttctgttccctccaaagccaaaggacaccctgatgatcagcagaacccc |
| | | tgaagtgacctgcgtggtggtggatgtgaagcacgaggacccagaagtgaa |
| | | gttcaattggtacgtggacggcgtggaagtgcacaacgccaagaccaagcct |
| | | agagaggaacagtacaacagcacctacagagtggtgtccgtgctgacagtg |
| | | ctgcaccaggactggctgaacggcaaagagtacaagtgcaaggtgtccaac |
| | | aaggccctggccgctcctatcgagaaaaccatctctaaggccaagggccag |
| | | cctcgggaacctcaagtgtgcactcttccacctagccgggaagagatgacca |
| | | agaaccaggtgtcactgagctgcgccgtgaagggcttctacccttctgatatcg |
| | | ccgtggaatgggagagcaacggccagccagagaacaactacaagaccac |
| | | acctcctgtgctggacagcgacggctcattcttcctggtgtctaagctgaccgtg |
| | | gacaagagcagatggcagcagggcaacgtgttcagctgtagcgtgatgcac |
| | | gaggccctgcacaaccactacacacagaagtccctgtctctgagccccggca |
| | | aa |
| 1129 | Anti-CD58 | sqqiygvvygnvtfhvpsnvplkevlwkkqkdkvaelensefrafssfknrvyl |
| | LALASKPA IgG1 | dtvsgsltiynltssdedeyemespnitdtmkfflyvlesggggsdkthtcppcp |
| | Heavy Chain | apeaaggpsvflfppkpkdtlmisrtpevtcvvvdvkhedpevkfnwyvdgv |
| | Amino acid | evhnaktkpreeqynstyrvvsvltvlhqdwlngkeykckvsnkalaapiekti |
| | sequence | skakgqprepqvytlppcreemtknqvslwclvkgfypsdiavewesngqp |
| | | ennykttppvldsdgsfflyskltvdksrwqqgnvfscsvmhealhnhytqksl |
| | | slspgk |
| 1130 | Anti-CD58 | tctcagcagatctatggcgtggtgtacggcaacgtgaccttccacgtgccaagc |
| | LALASKPA IgG1 | aacgtgcccctgaaagaggtgctctggaagaaacagaaagacaaggtggc |
| | Heavy Chain | cgagctggaaaacagcgagttcagagccttcagcagcttcaagaaccgggt |
| | Nucleic acid | gtacctggataccgtgtccggcagcctgaccatctacaatctgaccagcagcg |
| | sequence | acgaggacgagtacgagatggaaagccccaacatcaccgacaccatgaag |
| | | ttctttctgtacgtgctggaaagcggcggaggcggctctgataagacacacac |
| | | atgtcctccatgtcctgctccagaagcagctggcggaccctctgttttcctgtttcc |
| | | acctaagccaaaggacaccctgatgatcagcagaacccctgaagtgacctg |
| | | cgtggtggtggatgtgaagcacgaggaccccgaagtgaagttcaattggtacg |
| | | tggacggcgtggaagtgcacaacgccaagaccaagcctagagaggaaca |
| | | gtacaacagcacctacagagtggtgtccgtgctgaccgtgctgcaccaggatt |
| | | ggctgaacggcaaagagtacaagtgcaaggtgtccaacaaggccctggcc |
| | | gctcctatcgagaaaaccatctctaaggccaagggccagcctcgggaacca |
| | | caggtttacaccctgcctccatgccgggaagagatgaccaagaatcaggtgtc |
| | | cctgtggtgcctggtcaagggcttctacccttccgatatcgccgtggaatgggag |
| | | agcaatggccagcctgagaacaactacaagaccacacctcctgtgctggac |
| | | agcgacggctcattcttcctgtacagcaagctgacagtggacaagagcagat |
| | | ggcagcagggcaacgtgttcagctgtagcgtgatgcacgaggccctgcacaa |
| | | ccactacacccagaagtctctgtctctgagccccggcaaa |

### 8.38. Example 38: Biophysical Properties of Modified Antibodies: SPR-Binding of Modified Antibodies of Example 37 to Human Fc Gamma Receptor 1A

Surface plasmon resonance (SPR) experiments were performed to analyze the interaction of human activating receptor FcγR1A towards WT and antibody-Fc variants. Binding kinetics and their relative binding affinities were explored. The binding affinity is an important characteristic of an interaction between an antibody and an antigen. The equilibrium dissociation constant (K_{D}) defines how strong the interaction is and therefore how much antibody-antigen complex is formed at equilibrium. The knowledge of the antibody characteristics is not only essential during selection of the best therapeutic antibody candidate, but also important to understand the in vivo behavior and potentially predict cellular immune responses. The aim is to generate antibody variants with no or low binding to Fc gamma receptor to reduce or eliminate effector function in hope of improving the safety of monoclonal antibody therapeutics.

All SPR buffers were prepared using deionized water. The samples were prepared in running buffer PBS pH 7.4 with 0.005% Tween-20. SPR measurements were measured on a Biacore T200 (GE-Healthcare Life Sciences) controlled by Biacore T200 control software version 2.0.1.

Surface plasmon resonance was conducted using a Biacore T200 to assess binding affinity of antibody WT and variants to human FcγR1A.

The antibodies were covalently immobilized on a CM5 sensor chip whereas Fc gamma receptor 1A served as analyte in solution (FIG. 38). Antibodies were diluted in 10 mM sodium acetate pH 4 and immobilized at a density of approximately 950 resonance units (RU) on the CM5 sensor chip applying a standard amine coupling procedure. Flow cell 1 was blank immobilized to serve as a reference. The kinetic binding data were collected by subsequent injections of 1:2 dilution series of the human Fc gamma receptor 1A on all flow cells at a flow rate of 30 μl/min and at a temperature of 25° C. The Fc gamma receptor was diluted in the running buffer at concentrations ranging from 0.2 nM to 25 nM. The chip surface was regenerated using 20 mM glycine pH 2.0 solution after each measuring cycle. Zero concentration samples (blank runs) were measured to allow double-referencing during data evaluation.

Data were evaluated using the Biacore T200 evaluation software. The raw data were double referenced, i.e. the response of the measuring flow cell was corrected for the response of the reference flow cell, and in a second step the response of a blank injection was subtracted. Then the sensorgrams were fitted by applying a 1:1 kinetic binding model to calculate dissociation equilibrium constants. In addition, the maximum response reached during the experiment was monitored. Maximum response describes the binding capacity of the surface in terms of the response at saturation.

The maximum response values summarizing these interactions are given in Table 35.

The SPR Biacore binding sensorgrams for each variant towards FcγR1A were depicted in FIG. 41.

All variants inhibit the binding to Fc gamma receptors compare to WT as low residual binding was measured.

TABLE 35
Maximum responses of WT-Fc and variants towards
human FcγRIA determined by surface plasmon
resonance

| | Maximum response at 25nM hFcγRIA |
|---|---|
| Fc | (RU) |
| WT | 138 |
| DANAPA | 0 |
| GADAPASK | 0 |
| LALA | 44 |
| LALASKPA | 0 |

### Differential Scanning Calorimetry- Melting Temperature of Modified Antibodies

The thermal stability of engineered antibodies CH2 domains were compared using calorimetric measurements as shown in Table 36. Calorimetric measurements were carried out on a differential scanning micro calorimeter (Nano DSC, TA instruments). The cell volume was 1 ml and the heating rate was 1° C./min. All proteins were used at a concentration of 1 mg/mi in PBS (pH 7.4). The molar heat capacity of each protein was estimated by comparison with duplicate samples containing identical buffer from which the protein had been omitted. The partial molar heat capacities and melting curves were analyzed using standard procedure. Thermograms were baseline corrected and concentration normalized. The silent version LALASKPA (68° C.) shows significantly better Tm compared to DANAPA (57° C.).

TABLE 36
Melting temperatures of WT-Fc and variants

| Fc | Melting temperature (Tm) of CH2 domain |
|---|---|
| WT | 68 |
| DANAPA | 57 |
| GADAPASK | 59 |
| LALA | 68 |
| LALASKPA | 68 |

### Aggregation Propensity Post Capture of Fc Variants

Size exclusion chromatography measurements were performed to evaluate the aggregation propensity (% HMW) of WT and Fc modified derivatives. The produced and purified antibodies were applied to an analytical size exclusion chromatography column (SEC 200, GE Healthcare), equilibrated with PBS buffer pH 7.4. Results are summarized in Table 37.

TABLE 37
Higher molecular weight content (%) of antibodies

| Fc | (%) higher molecular weight |
|---|---|
| WT | 16.3 |
| DANAPA | 22.7 |
| GADAPASK | 16.1 |
| LALA | 18.1 |
| LALASKPA | 18.9 |

### 8.39. Example 39: Anti-CD3 NFAT Signalling Assay

Jurkat reporter gene assay (RGA) for the nuclear factor of activated T-cells (NFAT) pathway was performed using Jurkat NFAT luciferized (JNL) cells and THP-1 cells (ATCC, TIB202). THP-1 cells express FcγRI, FcγRII, and FcγRIII. Cells were co-incubated for 6 hours at 37° C., 5% CO2 at a 5:1 effector to tumor cell ratio with each sample at the various concentrations depicted. An equal volume of ONE-Glo™ reagent (Promega, E6120) was added to the culture volume. Plate was shaken for 2 minutes, then incubated for an additional 8 minutes protected from light. Luciferase activity was quantitated on the Biotek Synergy HT plate reader. Data were analyzed and fit to a 4 parameter-logistic curve using GraphPad Prism.

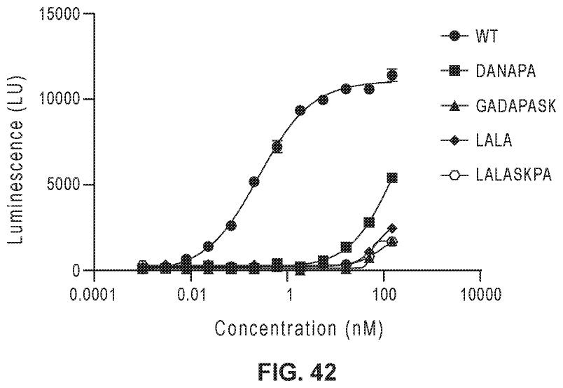

In summary, WT showed the greatest NFAT activity. All silencing mutation sets showed significantly dampened NFAT activation. In the RGA, performed without IFNg (FIG. 42), all silencing mutation sets showed comparable T-cell activation with the exception of DANAPA.

### 8.40. Example 40: CD31hi TSP1 Variant

A CD3hi TSP1 variant comprising knob-into-hole mutations in the opposite Fc regions as compared to CD3hi TSP1 was produced. The amino acid sequences of the variant are set forth in Table 38.

TABLE 38

| | | SEQ |
|---|---|---|
| Chain | | ID |
| Description | Amino Acid Sequence | NO |
| First Half | QVQLVQSGAEVKKPGASVKVSCKASGYTFTTYWIQWVRQAPGQRLE | 1131 |
| Antibody | WMGAVYPGDADTRYTQKFQGRVTLTADRSASTAYMELSSLRSEDTA | |
| Heavy | VYYCGRDAGLEYYALDYWGQGTLVTVSSASTKGPSVFPLAPSSKST | |
| Chain | SGGTAALGCLVKDYFPEPVTVSWNSGALTSGVHTFPAVLQSSGLYSL | |
| (includes Fc | SSVVTVPSSSLGTQTYICNVNHKPSNTKVDKRVEPKSCGGGGSGGG | |
| sequence) | GSEVQLVESGGGLVQPGGSLKLSCAASGFTFNTYAMNWVRQASGK | |
| | GLEWVGRIRSKYNNYATYYADSVKDRFTISRDDSKSTLYLQMNSLKT | |
| | EDTAVYYCVRHGNFGNSYVSWFAYWGQGTLVTVSSGGGGSGGGG | |
| | SGGGGSGGGGSQAVVTQEPSLTVSPGGTVTLTCRSSTGAVTTSNYA | |
| | NWVQQKPGQAPRGLIGGTNKRAPWTPARFSGSLLGDKAALTLSGAQ | |
| | PEDEAEYFCALWYSNLWVFGGGTKLTVLGGGGSDKTHTCPPCPAPE | |
| | LLGGPSVFLFPPKPKDTLMISRTPEVTCVVVAVSHEDPEVKFNWYVD | |
| | GVEVHNAKTKPREEQYASTYRVVSVLTVLHQDWLNGKEYKCKVSNK | |
| | ALAAPIEKTISKAKGQPREPQVYTLPPCREEMTKNQVSLWCLVKGFY | |
| | PSDIAVEWESNGQPENNYKTTPPVLDSDGSFFLYSKLTVDKSRWQQ | |
| | GNVFSCSVMHEALHNHYTQKSLSLSPGK | |
| First Half | EIVMTQSPATLSVSPGERATLSCRASQDVGTAVAWYQQKPGQAPRL | 1132 |
| Antibody | LIYWASTRHTGIPARFSGSGSGTEFTLTISSLQSEDFAVYFCQQYANF | |
| Light Chain | PLYTFGQGTKLEIKRTVAAPSVFIFPPSDEQLKSGTASVVCLLNNFYP | |
| | REAKVQWKVDNALQSGNSQESVTEQDSKDSTYSLSSTLTLSKADYE | |
| | KHKVYACEVTHQGLSSPVTKSFNRGEC | |
| Second Half | SQQIYGVVYGNVTFHVPSNVPLKEVLWKKQKDKVAELENSEFRAFSS | 1133 |
| Antibody | FKNRVYLDTVSGSLTIYNLTSSDEDEYEMESPNITDTMKFFLYVLESG | |
| (includes Fc | GGGSDKTHTCPPCPAPELLGGPSVFLFPPKPKDTLMISRTPEVTCVV | |
| sequence) | VAVSHEDPEVKFNWYVDGVEVHNAKTKPREEQYASTYRVVSVLTVL | |
| | HQDWLNGKEYKCKVSNKALAAPIEKTISKAKGQPREPQVCTLPPSRE | |
| | EMTKNQVSLSCAVKGFYPSDIAVEWESNGQPENNYKTTPPVLDSDG | |
| | SFFLVSKLTVDKSRWQQGNVFSCSVMHEALHNHYTQKSLSLSPGK | |

### 8.41. Example 41: CD3hi TSP1 Variants

Additional Fc variants of CD3hi TSP1 are designed, and expressed and purified according to the methods described in Example 37. Amino acid sequences of the variants are set forth in Table 39.

TABLE 39
Exemplary TBM sequences

| SEQ ID | | |
|---|---|---|
| No | Description | Sequence |
| 1110 | Anti-CD19 light | eivmtqspatlsvspgeratlscrasqdvgtavawyqqkpgqaprlliywastr |
| | chain | htgiparfsgsgsgteftltisslqsedfavyfcqqyanfplytfgqgtkleikrtvaa |
| | Amino acid | psvfifppsdeqlksgtasvvcllnnfypreakvqwkvdnalqsgnsqesvteq |
| | sequence | dskdstyslsstltlskadyekhkvyacevthqglsspvtksfnrgec |
| 1134 | Anti-CD19-CD3 | qvqlvqsgaevkkpgasvkvsckasgytfttywiqwvrqapgqrlewmgav |
| | LALAGA IgG1 | ypgdadtrytqkfqgrvtltadrsastaymelsslrsedtavyycgrdagleyyal |
| | Heavy Chain | dywgqgtlvtvssastkgpsvfplapsskstsggtaalgclvkdyfpepvtvsw |
| | Amino acid | nsgaltsgvhtfpavlqssglyslssvvtvpssslgtqtyicnvnhkpsntkvdkr |
| | sequence | vepkscggggsggggsevqlvesggglvqpggslklscaasgftfntyamn |
| | | wvrqasgkglewvgrirskynnyatyyadsvkdrftisrddskstlylqmnslkt |
| | | edtavyycvrhgnfgnsyvswfaywgqgtlvtvssggggsggggsggggsg |
| | | gggsqavvtqepsltvspggtvtltcrsstgavttsnyanwvqqkpgqaprglig |
| | | gtnkrapwtparfsgsllgdkaaltlsgaqpedeaeyfcalwysnlwvfgggtkl |
| | | tvlggggsdkthtcppcpapeaagapsvflfppkpkdtlmisrtpevtcvvvdv |
| | | shedpevkfnwyvdgvevhnaktkpreeqynstyrvvsvltvlhqdwlngke |
| | | ykckvsnkalpapiektiskakgqprepqvctlppsreemtknqvslscavkgf |
| | | ypsdiavewesngqpennykttppvldsdgsfflvskltvdksrwqqgnvfsc |
| | | svmhealhnhytqkslslspgk |
| 1135 | Anti-CD58 | sqqiygvvygnvtfhvpsnvplkevlwkkqkdkvaelensefrafssfknrvyl |
| | LALAGA IgG1 | dtvsgsltiynltssdedeyemespnitdtmkfflyvlesggggsdkthtcppcp |
| | Heavy Chain | apeaagapsvflfppkpkdtlmisrtpevtcvvvdvshedpevkfnwyvdgv |
| | Amino acid | evhnaktkpreeqynstyrvvsvltvlhqdwlngkeykckvsnkalpapiekti |
| | sequence | skakgqprepqvytlppcreemtknqvslwclvkgfypsdiavewesngqp |
| | | ennykttppvldsdgsfflyskltvdksrwqqgnvfscsvmhealhnhytqksl |
| | | slspgk |
| 1136 | Anti-CD19-CD3 | qvqlvqsgaevkkpgasvkvsckasgytfttywiqwvrqapgqrlewmgav |
| | DAPASK IgG1 | ypgdadtrytqkfqgrvtltadrsastaymelsslrsedtavyycgrdagleyyal |
| | Heavy Chain | dywgqgtlvtvssastkgpsvfplapsskstsggtaalgclvkdyfpepvtvsw |
| | Amino acid | nsgaltsgvhtfpavlqssglyslssvvtvpssslgtqtyicnvnhkpsntkvdkr |
| | sequence | vepkscggggsggggsevqlvesggglvqpggslklscaasgftfntyamn |
| | | wvrqasgkglewvgrirskynnyatyyadsvkdrftisrddskstlylqmnslkt |
| | | edtavyycvrhgnfgnsyvswfaywgqgtlvtvssggggsggggsggggsg |
| | | gggsqavvtqepsltvspggtvtltcrsstgavttsnyanwvqqkpgqaprglig |
| | | gtnkrapwtparfsgsllgdkaaltlsgaqpedeaeyfcalwysnlwvfgggtkl |
| | | tvlggggsdkthtcppcpapellggpsvflfppkpkdtlmisrtpevtcvvvavk |
| | | hedpevkfnwyvdgvevhnaktkpreeqynstyrvvsvltvlhqdwlngkey |
| | | kckvsnkalaapiektiskakgqprepqvctlppsreemtknqvslscavkgfy |
| | | psdiavewesngqpennykttppvldsdgsfflvskltvdksrwqqgnvfscs |
| | | vmhealhnhytqkslslspgk |
| 1137 | Anti-CD58 | sqqiygvvygnvtfhvpsnvplkevlwkkqkdkvaelensefrafssfknrvyl |
| | DAPASK IgG1 | dtvsgsltiynltssdedeyemespnitdtmkfflyvlesggggsdkthtcppcp |
| | Heavy Chain | apellggpsvflfppkpkdtlmisrtpevtcvvvavkhedpevkfnwyvdgvev |
| | Amino acid | hnaktkpreeqynstyrvvsvltvlhqdwlngkeykckvsnkalaapiektisk |
| | sequence | akgqprepqvytlppcreemtknqvslwclvkgfypsdiavewesngqpen |
| | | nykttppvldsdgsfflyskltvdksrwqqgnvfscsvmhealhnhytqkslsls |
| | | pgk |
| 1138 | Anti-CD19-CD3 | qvqlvqsgaevkkpgasvkvsckasgytfttywiqwvrqapgqrlewmgav |
| | GADAPA IgG1 | ypgdadtrytqkfqgrvtltadrsastaymelsslrsedtavyycgrdagleyyal |
| | Heavy Chain | dywgqgtlvtvssastkgpsvfplapsskstsggtaalgclvkdyfpepvtvsw |
| | Amino acid | nsgaltsgvhtfpavlqssglyslssvvtvpssslgtqtyicnvnhkpsntkvdkr |
| | sequence | vepkscggggggggsevqlvesggglvqpggslklscaasgftfntyamn |
| | | wvrqasgkglewvgrirskynnyatyyadsvkdrftisrddskstlylqmnslkt |
| | | edtavyycvrhgnfgnsyvswfaywgqgtlvtvssggggsggggsggggsg |
| | | gggsqavvtqepsltvspggtvtltcrsstgavttsnyanwvqqkpgqaprglig |
| | | gtnkrapwtparfsgsllgdkaaltlsgaqpedeaeyfcalwysnlwvfgggtkl |
| | | tvlggggsdkthtcppcpapellgapsvflfppkpkdtlmisrtpevtcvvvavs |
| | | hedpevkfnwyvdgvevhnaktkpreeqynstyrvvsvltvlhqdwlngkey |
| | | kckvsnkalaapiektiskakgqprepqvctlppsreemtknqvslscavkgfy |
| | | psdiavewesngqpennykttppvldsdgsfflvskltvdksrwqqgnvfscs |
| | | vmhealhnhytqkslslspgk |
| 1139 | Anti-CD58 | sqqiygvvygnvtfhvpsnvplkevlwkkqkdkvaelensefrafssfknrvyl |
| | GADAPA IgG1 | dtvsgsltiynltssdedeyemespnitdtmkfflyvlesggggsdkthtcppcp |
| | Heavy Chain | apellgapsvflfppkpkdtlmisrtpevtcvvvavshedpevkfnwyvdgvev |
| | Amino acid | hnaktkpreeqynstyrvvsvltvlhqdwlngkeykckvsnkalaapiektisk |
| | sequence | akgqprepqvytlppcreemtknqvslwclvkgfypsdiavewesngqpen |
| | | nykttppvldsdgsfflyskltvdksrwqqgnvfscsvmhealhnhytqkslsls |
| | | pgk |
| 1140 | Anti-CD19-CD3 | qvqlvqsgaevkkpgasvkvsckasgytfttywiqwvrqapgqrlewmgav |
| | LALAPG IgG1 | ypgdadtrytqkfqgrvtltadrsastaymelsslrsedtavyycgrdagleyyal |
| | Heavy Chain | dywgqgtlvtvssastkgpsvfplapsskstsggtaalgclvkdyfpepvtvsw |
| | Amino acid | nsgaltsgvhtfpavlqssglyslssvvtvpssslgtqtyicnvnhkpsntkvdkr |
| | sequence | vepkscggggsggggsevalvesggglvqpggslklscaasgftfntyamn |
| | | wvrqasgkglewvgrirskynnyatyyadsvkdrftisrddskstlylqmnslkt |
| | | edtavyycvrhgnfgnsyvswfaywgqgtlvtvssggggsggggsggggsg |
| | | gggsqavvtqepsltvspggtvtltcrsstgavttsnyanwvqqkpgqaprglig |
| | | gtnkrapwtparfsgsllgdkaaltlsgaqpedeaeyfcalwysnlwvfgggtkl |
| | | tvlggggsdkthtcppcpapeaaggpsvflfppkpkdtlmisrtpevtcvvvdv |
| | | shedpevkfnwyvdgvevhnaktkpreeqynstyrvvsvltvlhqdwlngke |
| | | ykckvsnkalgapiektiskakgqprepqvctlppsreemtknqvslscavkgf |
| | | ypsdiavewesngqpennykttppvldsdgsfflvskltvdksrwqqgnvfsc |
| | | svmhealhnhytqkslslspgk |
| 1141 | Anti-CD58 | sqqiygvvygnvtfhvpsnvplkevlwkkqkdkvaelensefrafssfknrvyl |
| | LALAPG IgG1 | dtvsgsltiynltssdedeyemespnitdtmkfflyvlesggggsdkthtcppcp |
| | Heavy Chain | apeaaggpsvflfppkpkdtlmisrtpevtcvvvdvshedpevkfnwyvdgv |
| | Amino acid | evhnaktkpreeqynstyrvvsvltvlhqdwlngkeykckvsnkalgapiekti |
| | sequence | skakgqprepqvytlppcreemtknqvslwclvkgfypsdiavewesngqp |
| | | ennykttppvldsdgsfflyskltvdksrwqqgnvfscsvmhealhnhytqksl |
| | | slspgk |
| 1142 | Anti-CD19-CD3 | qvqlvqsgaevkkpgasvkvsckasgytfttywiqwvrqapgqrlewmgav |
| | LALAPA IgG1 | ypgdadtrytqkfqgrvtltadrsastaymelsslrsedtavyycgrdagleyyal |
| | Heavy Chain | dywgqgtlvtvssastkgpsvfplapsskstsggtaalgclvkdyfpepvtvsw |
| | Amino acid | nsgaltsgvhtfpavlqssglyslssvvtvpssslgtqtyicnvnhkpsntkvdkr |
| | sequence | vepkscggggsggggsevalvesggglvqpggslklscaasgftfntyamn |
| | | wvrqasgkglewvgrirskynnyatyyadsvkdrftisrddskstlylqmnslkt |
| | | edtavyycvrhgnfgnsyvswfaywgqgtlvtvssggggsggggsggggsg |
| | | gggsqavvtqepsltvspggtvtltcrsstgavttsnyanwvqqkpgqaprglig |
| | | gtnkrapwtparfsgsllgdkaaltlsgaqpedeaeyfcalwysnlwvfgggtkl |
| | | tvlggggsdkthtcppcpapeaaggpsvflfppkpkdtlmisrtpevtcvvvdv |
| | | shedpevkfnwyvdgvevhnaktkpreeqynstyrvvsvltvlhqdwlngke |
| | | ykckvsnkalaapiektiskakgqprepqvctlppsreemtknqvslscavkgf |
| | | ypsdiavewesngqpennykttppvldsdgsfflvskltvdksrwqqgnvfsc |
| | | svmhealhnhytqkslslspgk |
| 1143 | Anti-CD58 | sqqiygvvygnvtfhvpsnvplkevlwkkqkdkvaelensefrafssfknrvyl |
| | LALAPA IgG1 | dtvsgsltiynltssdedeyemespnitdtmkfflyvlesggggsdkthtcppcp |
| | Heavy Chain | apeaaggpsvflfppkpkdtlmisrtpevtcvvvdvshedpevkfnwyvdgv |
| | Amino acid | evhnaktkpreeqynstyrvvsvltvlhqdwlngkeykckvsnkalaapiekti |
| | sequence | skakgqprepqvytlppcreemtknqvslwclvkgfypsdiavewesngqp |
| | | ennykttppvldsdgsfflyskltvdksrwqqgnvfscsvmhealhnhytqksl |
| | | slspgk |

### 8.42. Example 42: Depletion of Normal B Cells by Ianalumab in Mouse

The effect of ianalumab on healthy B-cell levels in mice was evaluated in a repeat dose toxicity study.

### 8.42.1. Materials and Methods

CD-1 mice were administered 0 mg/kg or 100 mg/kg of ianalumab by intravenous administration weekly for 13 weeks, followed by an 11 week recovery period.

### 8.42.2. Results

In CD-1 mice administered 100 mg/kg of ianalumab, 70-90% of mature B cell depletion was observed. B cell levels recovered during the recovery period.

### 8.43. Example 43: Depletion of Normal B Cells by Ianalumab in Cynomolgus Monkey

A rising single i.v. dose range finding (DRF), toxicity and TK/PD study and three repeated dose toxicity studies were performed with ianalumab in cynomolgus monkeys. B-cell levels were evaluated in the studies.

In the single dose study, ianalumab at doses of 0.4 mg/kg and higher induced depletion of B cells. lanalumab was well tolerated.

Across three repeated dose studies, B cell depletion was observed at all dose levels.

Taken together, the mouse and cynomolgus monkey studies show that ianalumab depletes healthy B-cells in vivo. A similar effect of ianalumab on healthy B-cell cells in human is expected.

### 8.44. Example 44: In-Vitro Cytokine Release from B Cell Depleted PBMC-Karpas 422 or T Cell-Karpas 422 Co-Cultures with B Cell Titration

The impact of B cells on cytokine secretion induced by CD3hi TSP1 was evaluated by adding increasing numbers of B cells into B cell depleted PBMC-Karpas 422 or T cell-Karpas 422 co-culture systems.

### 8.44.1. Materials and Methods

Karpas 422 cells stably expressing firefly luciferase were plated in a 96 well plate in T Cell Media (TCM) [RPMI-1640 (ThermoFisher Scientific, Cat #11875-085), 10% FBS (Seradigm, Cat #1500-500), 1% L-Glutamine (Thermo Fisher Scientific, Cat #25830-081), 1% Non Essential Amino Acids (Thermo Fisher Scientific, Cat #11140-050), 1% Pen/Strep (Thermo Fisher Scientific, Cat #15070063), 1% HEPES (Thermo Fisher Scientific, Cat #15630080), Sodium Pyruvate (Thermo Fisher Scientific, Cat #11360-070), 0.1% Beta-mercaptoethanol (Thermo Fisher Scientific, Cat #21985-023)]. Peripheral blood mononuclear cells (PBMCs) previously isolated and cryopreserved from 2 Leukopak donors (Hemacare) were thawed and B cells were isolated via positive selection using the REAlease CD19 Microbead Kit [Miltenyi Biotec, Cat #130-117-034] following the manufacturer's protocol. The cells in the flowthrough represent the B cell depleted PBMC fraction, which was co-cultured with Karpas 422 cells at an E:T ratio of 2.5:1. Increasing numbers of the isolated B cells were then added to the co-culture. A dilution series of CD3hi TSP1 (H variant) antibody ranging from 10 nM-0.00001 nM was added to cells and the plates were incubated in a 5% C02, 37° C. incubator for 48 hrs. After 48 hrs, cell supernatants were harvested, diluted 5 fold and a multiplexed ELISA was performed according to the manufacturer's instructions using the V-PLEX Proinflammatory Panel 1 Human Kit (MesoScale Discovery #K15049D-4). In another iteration of the assay, a co-culture between isolated T cells and Karpas 422 cells was set-up at the E:T ratio of 1:1 with the remainder of the set-up remaining same.

### 8.44.2. Results

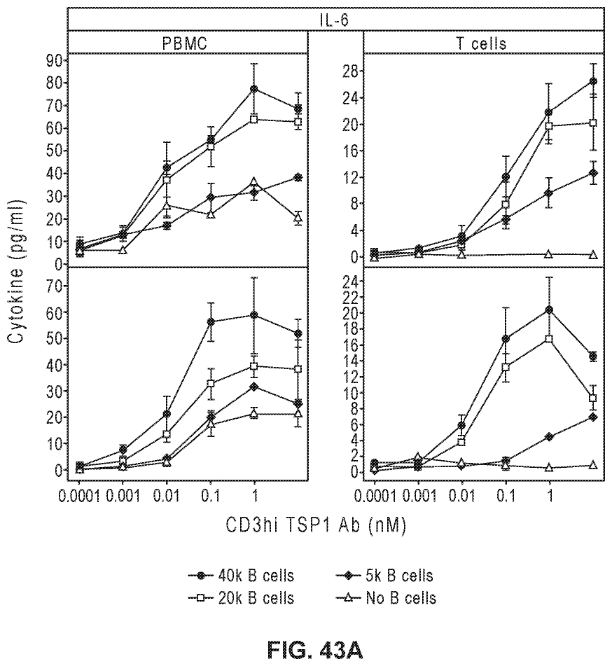

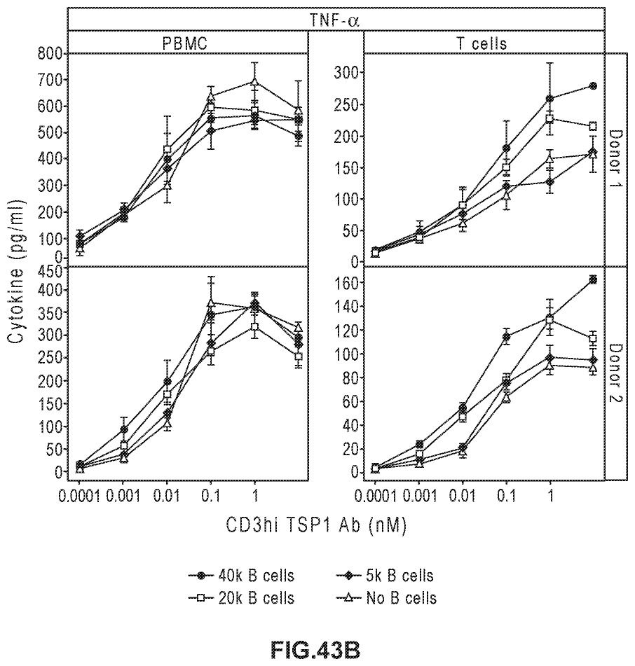

A dose dependent increase in cytokine secretion was observed in all co-culture conditions (FIGS. 43A-43B). IL-6 and TNFα are described in the literature as being important contributors to CRS (Ji et al., 2019, Sci. Transl. Med. 11:eaax8861). As compared to the co-culture condition with no B cells, an increase in the absolute amount of IL-6 (FIG. 43A) and TNFα (FIG. 43B) secreted was observed with addition of increasing numbers of B cells.

### 8.45. Example 45: BAFF-R Expression on B-Cell Lymphoma Cell Lines

Flow cytometric analysis was performed on a panel of lymphoma cell lines to ascertain the surface expression of CD19 and BAFF-R.

### 8.45.1. Materials and Methods

Cell surface expression of BAFF-R and CD19 on luciferized DOHH-2, Karpas 422 cells, OCILY-19, SUDHL-4 and Toledo cells was determined by flow cytometry using APC labelled anti-BAFF-R (Biolegend, Cat #316916) and FITC-labelled anti-CD19 (Biolegend, Cat #302206) antibodies. Data was acquired on BD FACSCanto and analysed using FlowJo.

### 8.45.2. Results

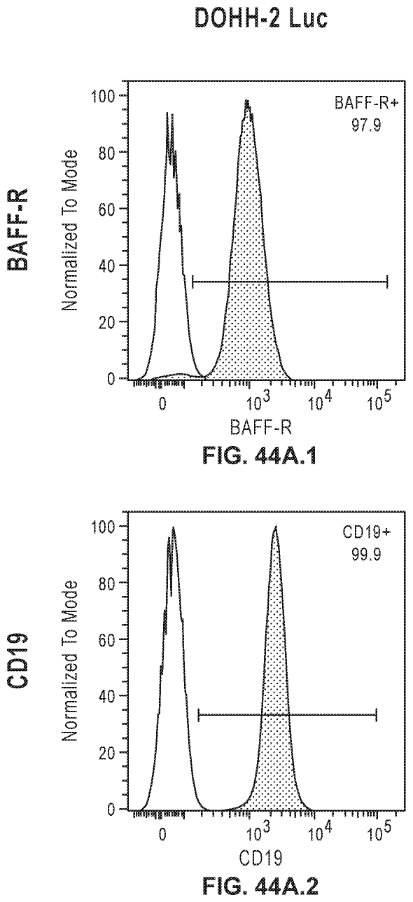

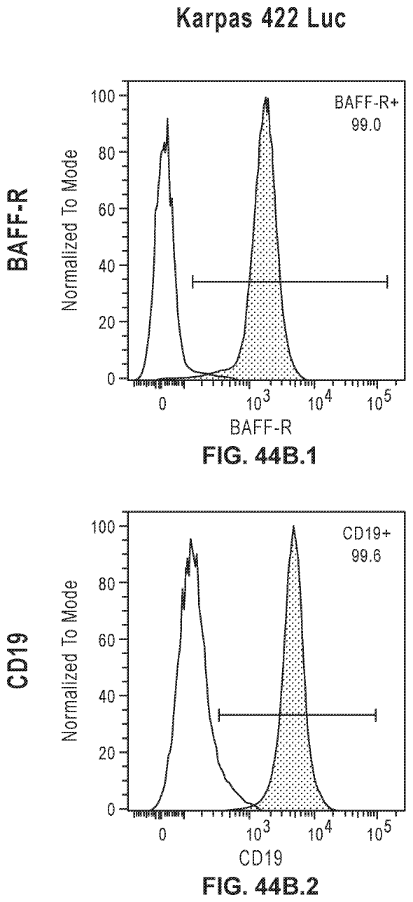

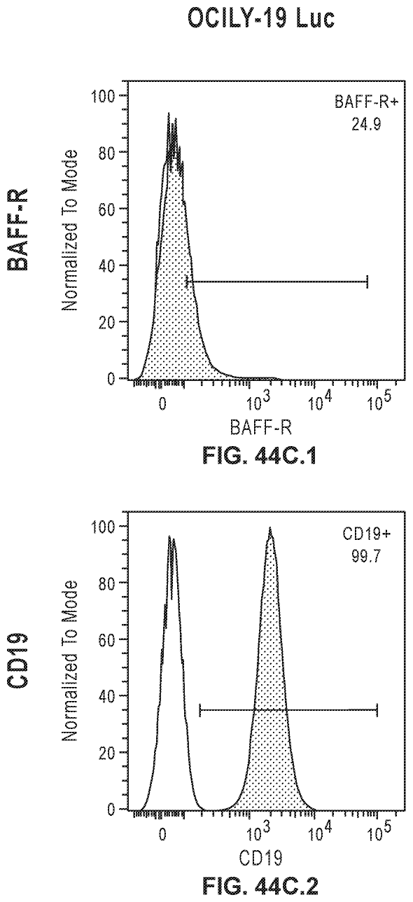

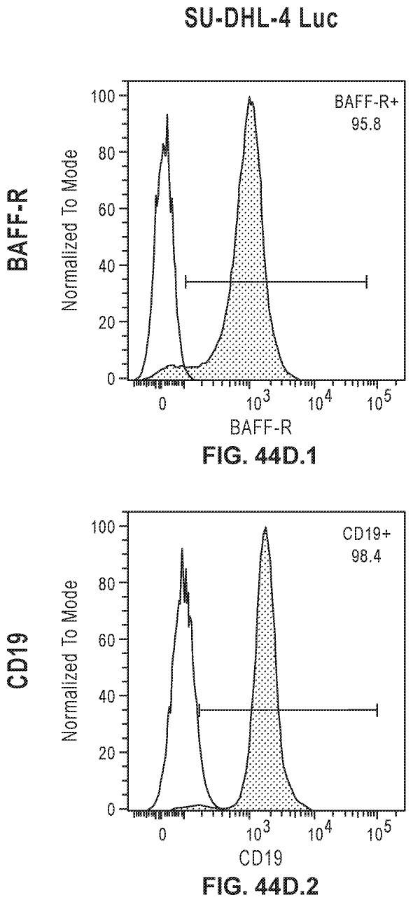

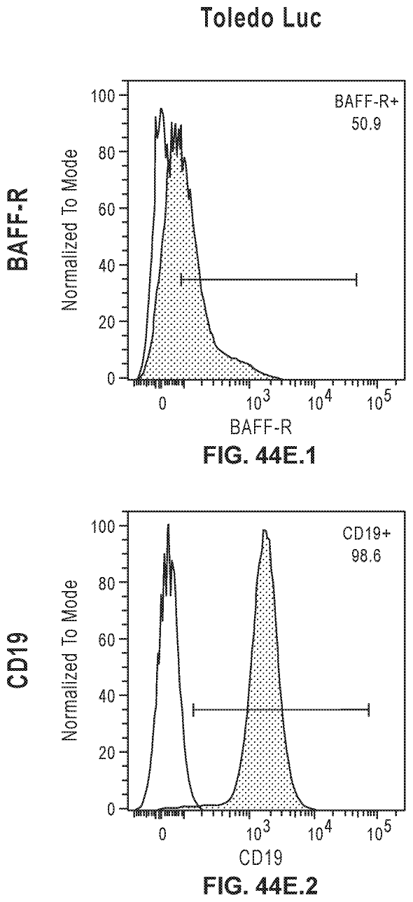

The various cell lines had different levels of BAFF-R and CD19 expression (FIGS. 44A.1-44E.2). Karpas 422 cells were found to express high levels of both BAFF-R and CD19 (FIGS. 44B.1-44B.2).

### 8.46. Example 46: CD3hi TSP1-VAY736 Anti-Tumor Combination Activity

The combined anti-tumor activity of an anti-BAFF-R antibody and CD3hi TSP1 antibody was assessed by co-culturing Karpas 422 cells with B cell depleted PBMCs.

### 8.46.1. Materials and Methods

Karpas 422 cells stably expressing firefly luciferase were plated in a 96 well plate in NK Media (NKM) [RPMI-1640 (ThermoFisher Scientific, Cat #11835030), 10% Ultra low IgG FBS (ThermoFisher Scientific, Cat #A3381901), 1% HEPES (Thermo Fisher Scientific, Cat #15630080), 0.1% Beta-mercaptoethanol (Thermo Fisher Scientific, Cat #21985-023)]. Peripheral blood mononuclear cells (PBMCs) previously isolated and cryopreserved from 2 Leukopak donors (StemCell Technologies) were thawed and B cells were depleted via positive selection using the REAlease CD19 Microbead Kit [Miltenyi Biotec, Cat #130-117-034] following the manufacturer's protocol. The cells in the flowthrough represent the B cell depleted PBMC fraction, which was co-cultured with Karpas 422 cells at an E:T ratio of 20:1. A dilution series of anti-BAFF-R antibody VAY736 or a non-targeting control antibody (Afuc) ranging from 1000 ng/ml-0.01 ng/ml was added to cells and the plates were incubated in a 5% C02, 370C incubator for 24 hrs. After 24 hrs a dilution series of CD3hi TSP1 Ab (H variant) or CD3hi TSP1C antibody ranging from 0.1 nM-0.0001 nM was added to cells and the plates were incubated in a 5% C02, 37° C. incubator another 48 hrs. At the end of the incubation, BrightGlo luciferase substrate (Promega #E2650) was added to the plate and luminescence was measured on an Envision plate reader after a 5 minute incubation on a shaker. Percent specific lysis was calculated using the following equation:

$$Specific lysis ( \% ) = ( 1 - ( sample luminescence / average maximum luminescence ) ) * 100$$

### 8.46.2. Results

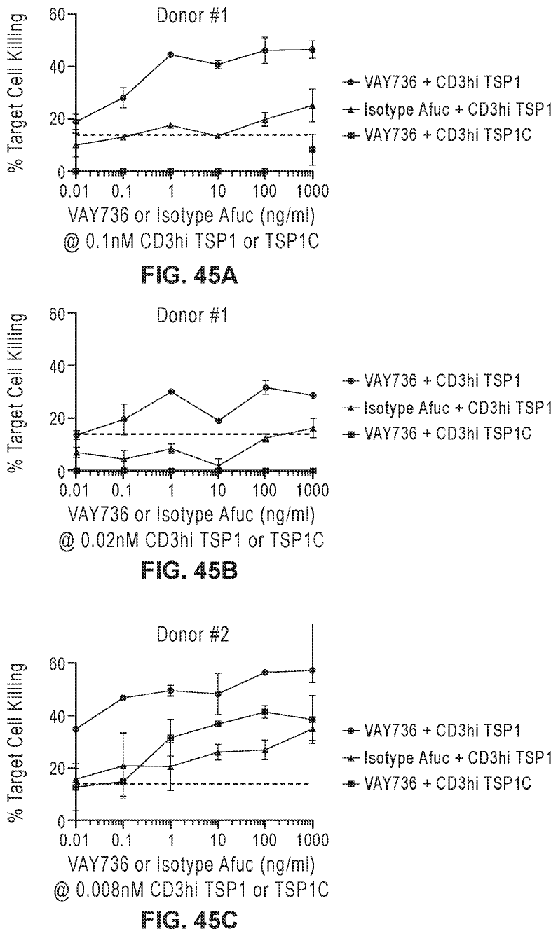

Cells from different donors have different sensitivity to CD3hi TSP1 and hence different concentrations of CD3hi TSP1 were chosen for evaluation of the combination of CD3hi TSP1 and VAY736. In each case, a submaximal concentration of CD3hi TSP1 was chosen for evaluation of the combination. The combination of VAY736 and CD3hi TSP1 antibody demonstrated greater anti-tumor activity than the control conditions (either the combination of anti-BAFF-R and CD3hi TSP1C (isotype control) antibody or the combination of Isotype and CD3hi TSP1 antibody) (FIGS. 45A-45C).

### 8.47. Example 47: VAY736 Slows Tumor Growth in DLBCL Model

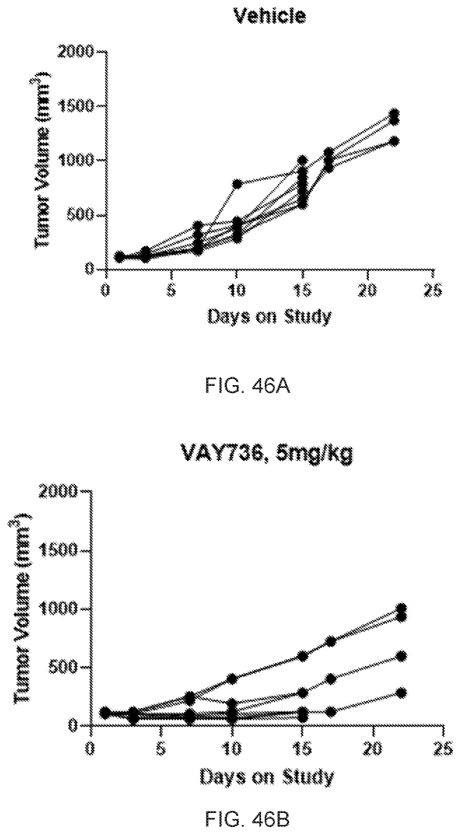

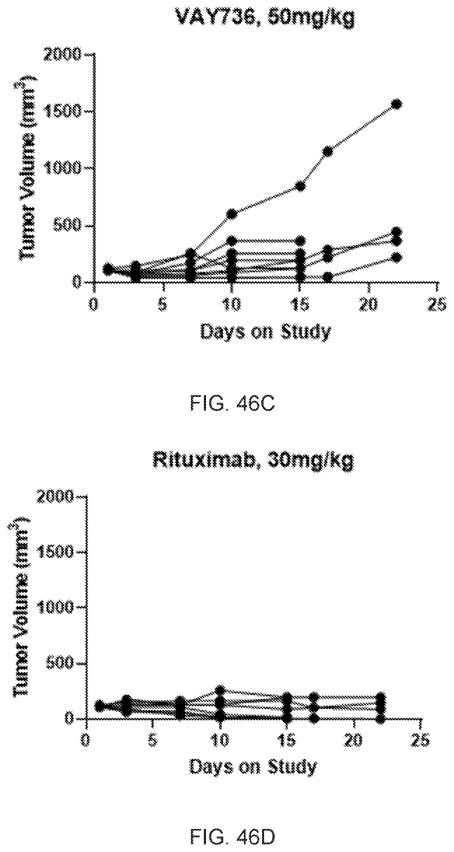

A study was performed to assess the effect of VAY736 in an in vivo DLBCL model. Briefly, the DLBCL cell line SUDHL4 was implanted subcutaneously into SCID mice, which were then treated weekly with 5 mg/kg or 50 mg/kg VAY736 intravenously. Vehicle and rituximab were used as controls. As shown in FIGS. 46A-46C, VAY736 treatment significantly slowed tumor growth in the model compared to the vehicle control, as assessed by tumor volume measurements collected over time.

### 9. SPECIFIC EMBODIMENTS, CITATION OF REFERENCES

While various specific embodiments have been illustrated and described, it will be appreciated that various changes can be made without departing from the spirit and scope of the disclosure(s).

The present disclosure is exemplified by the numbered embodiments set forth below.
1. A combination comprising:
   (a) an anti-CD19 agent; and
   (b) a B cell targeting agent.
2. The combination of embodiment 1, wherein anti-CD19 agent is a CD19 binding molecule.
3. The combination of embodiment 2, wherein the CD19 binding molecule comprises CDR-H1, CDR-H2, and CDR-H3 having the amino acid sequences of SEQ ID NO:1, SEQ ID NO:2, and SEQ ID NO:3, and CDR-L1, CDR-L2, and CDR-L3 having the amino acid sequences of SEQ ID NO:14, SEQ ID NO:15, and SEQ ID NO:16.
4. The combination of embodiment 2, wherein the CD19 binding molecule comprises CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19.
5. The combination of embodiment 2, wherein the CD19 binding molecule comprises CDR-H1, CDR-H2, and CDR-H3 having the amino acid sequences of SEQ ID NO:7, SEQ ID NO:8, and SEQ ID NO:9, and CDR-L1, CDR-L2, and CDR-L3 having the amino acid sequences of SEQ ID NO:20, SEQ ID NO:21, and SEQ ID NO:22.
6. The combination of embodiment 2, wherein the CD19 binding molecule comprises CDR-H1, CDR-H2, and CDR-H3 having the amino acid sequences of SEQ ID NO:10, SEQ ID NO:11, and SEQ ID NO:12, and CDR-L1, CDR-L2, and CDR-L3 having the amino acid sequences of SEQ ID NO:23, SEQ ID NO:24, and SEQ ID NO:25.
7. The combination of any one of embodiments 3 to 6, wherein the CD19 binding molecule comprises a VH having the amino acid sequence of SEQ ID NO:13.
8. The combination of any one of embodiments 3 to 7, wherein the CD19 binding molecule comprises a VL having the amino acid sequence of SEQ ID NO:26.
9. The combination of embodiment 2, wherein the CD19 binding molecule comprises CDR-H1, CDR-H2, and CDR-H3 having the amino acid sequences of SEQ ID NO:27, SEQ ID NO:28, and SEQ ID NO:29, and CDR-L1, CDR-L2, and CDR-L3 having the amino acid sequences of SEQ ID NO:40, SEQ ID NO:41, and SEQ ID NO:42.
10. The combination of embodiment 2, wherein the CD19 binding molecule comprises CDR-H1, CDR-H2, and CDR-H3 having the amino acid sequences of SEQ ID NO:30, SEQ ID NO:31, and SEQ ID NO:32, and CDR-L1, CDR-L2, and CDR-L3 having the amino acid sequences of SEQ ID NO:43, SEQ ID NO:44, and SEQ ID NO:45.
11. The combination of embodiment 2, wherein the CD19 binding molecule comprises CDR-H1, CDR-H2, and CDR-H3 having the amino acid sequences of SEQ ID NO:33, SEQ ID NO:34, and SEQ ID NO:35, and CDR-L1, CDR-L2, and CDR-L3 having the amino acid sequences of SEQ ID NO:46, SEQ ID NO:47, and SEQ ID NO:48.
12. The combination of embodiment 2, wherein the CD19 binding molecule comprises CDR-H1, CDR-H2, and CDR-H3 having the amino acid sequences of SEQ ID NO:36, SEQ ID NO:37, and SEQ ID NO:38, and CDR-L1, CDR-L2, and CDR-L3 having the amino acid sequences of SEQ ID NO:49, SEQ ID NO:50, and SEQ ID NO:51.
13. The combination of any one of embodiments 9 to 12, wherein the CD19 binding molecule comprises a VH having the amino acid sequence of SEQ ID NO:39.
14. The combination of any one of embodiments 9 to 13, wherein the CD19 binding molecule comprises a VL having the amino acid sequence of SEQ ID NO:52.
15. The combination of embodiment 2, wherein the CD19 binding molecule comprises:
   (a) a CDR-H1 having the amino acid sequence of the CDR designated as CD19-H1 in Table 1C;
   (b) a CDR-H2 having the amino acid sequence of any one of the CDRs designated as CD19-H2A, HD19-H2B, CD19-H2C and CD19-H2D in Table 1C;
   (c) a CDR-H3 having the amino acid sequence of the CDR designated as CD19-H3 in Table 1C;
   (d) a CDR-L1 having the amino acid sequence of the CDR designated as CD19-L1 in Table 1C;
   (e) a CDR-L2 having the amino acid sequence of the CDR designated as CD19-L2 in Table 1C; and
   (f) a CDR-L3 having the amino acid sequence of the CDR designated as CD19-L23 in Table 1C.
16. The combination of embodiment 15, wherein the CD19 binding molecule comprises:
   (a) a VH having the amino acid sequence of any one of the VH domains designated as CD19-VHA, CD19-VHB, CD19-VHC, and CD19-VHD in Table 1C; and
   (b) a VL having the amino acid sequence of any one of the VL domains designated as CD19-VLA and CD19-VLB in Table 1C.
17. The combination of embodiment 15, wherein the CD19 binding molecule comprises heavy chain CDRs having the amino acid sequences of CD19-H1, CD19-H2A, and CD19-H3 as set forth in Table 1C and light chain CDRs having the amino acid sequences of CD19-L1, CD19-L2, and CD19-L3 as set forth in Table 1C.
18. The combination of embodiment 15, wherein the CD19 binding molecule comprises a heavy chain variable region having the amino acid sequences of VHA as set forth in Table 1C and a light chain variable region having the amino acid sequences of VLA as set forth in Table 1C.
19. The combination of embodiment 15, wherein the CD19 binding molecule comprises heavy chain CDRs having the amino acid sequences of CD19-H1, CD19-H2B, and CD19-H3 as set forth in Table 1C and light chain CDRs having the amino acid sequences of CD19-L1, CD19-L2, and CD19-L3 as set forth in Table 1C.
20. The combination of embodiment 15, wherein the CD19 binding molecule comprises a heavy chain variable region having the amino acid sequences of VHB as set forth in Table 1C and a light chain variable region having the amino acid sequences of VLB as set forth in Table 1C.
21. The combination of embodiment 15, wherein the CD19 binding molecule comprises heavy chain CDRs having the amino acid sequences of CD19-H1, CD19-H2C, and CD19-H3 as set forth in Table 1C and light chain CDRs having the amino acid sequences of CD19-L1, CD19-L2, and CD19-L3 as set forth in Table 1C.
22. The combination of embodiment 15, wherein the CD19 binding molecule comprises a heavy chain variable region having the amino acid sequences of VHC as set forth in Table 1C and a light chain variable region having the amino acid sequences of VLB as set forth in Table 1C.
23. The combination of embodiment 15, wherein the CD19 binding molecule comprises heavy chain CDRs having the amino acid sequences of CD19-H1, CD19-H2D, and CD19-H3 as set forth in Table 1C and light chain CDRs having the amino acid sequences of CD19-L1, CD19-L2, and CD19-L3 as set forth in Table 1C.
24. The combination of embodiment 15, wherein the CD19 binding molecule comprises a heavy chain variable region having the amino acid sequences of VHD as set forth in Table 1C and a light chain variable region having the amino acid sequences of VLB as set forth in Table 1C.
25. The combination of embodiment 15, wherein the CD19 binding molecule comprises a scFv comprising the amino acid sequence of CD19-scFv1 as set forth in Table 1C.
26. The combination of embodiment 15, wherein the CD19 binding molecule comprises a scFv comprising the amino acid sequence of CD19-scFv2 as set forth in Table 1C.
27. The combination of embodiment 15, wherein the CD19 binding molecule comprises a scFv comprising the amino acid sequence of CD19-scFv3 as set forth in Table 1C.
28. The combination of embodiment 15, wherein the CD19 binding molecule comprises a scFv comprising the amino acid sequence of CD19-scFv4 as set forth in Table 1C.
29. The combination of embodiment 15, wherein the CD19 binding molecule comprises a scFv comprising the amino acid sequence of CD19-scFv5 as set forth in Table 1C.
30. The combination of embodiment 15, wherein the CD19 binding molecule comprises a scFv comprising the amino acid sequence of CD19-scFv6 as set forth in Table 1C.
31. The combination of embodiment 15, wherein the CD19 binding molecule comprises a scFv comprising the amino acid sequence of CD19-scFv7 as set forth in Table 1C.
32. The combination of embodiment 15, wherein the CD19 binding molecule comprises a scFv comprising the amino acid sequence of CD19-scFv8 as set forth in Table 1C.
33. The combination of embodiment 15, wherein the CD19 binding molecule comprises a scFv comprising the amino acid sequence of CD19-scFv9 as set forth in Table 1C.
34. The combination of embodiment 15, wherein the CD19 binding molecule comprises a scFv comprising the amino acid sequence of CD19-scFv10 as set forth in Table 1C.
35. The combination of embodiment 15, wherein the CD19 binding molecule comprises a scFv comprising the amino acid sequence of CD19-scFv11 as set forth in Table 1C.
36. The combination of embodiment 15, wherein the CD19 binding molecule comprises a scFv comprising the amino acid sequence of CD19-scFv12 as set forth in Table 1C.
37. The combination of any one of embodiments 2 to 24, wherein the CD19 binding molecule comprises an antibody, an antibody fragment, an scFv, a dsFv, a Fv, a Fab, an scFab, a (Fab′)2, or a single domain antibody (SDAB).
38. The combination of embodiment 37, wherein the CD19 binding molecule comprises an antibody or an antigen-binding domain thereof.
39. The combination of any one of embodiments 2 to 38, wherein the CD19 binding molecule is a monospecific binding molecule.
40. The combination of any one of embodiments 2 to 38, wherein the CD19 binding molecule is a multispecific binding molecule (MBM).
41. The combination of embodiment 40, wherein the CD19 binding molecule comprises
   (a) an antigen-binding module 1 (ABM1) that binds specifically to CD19; and
   (b) an antigen-binding module 2 (ABM2) that binds specifically to a different target molecule, optionally wherein the target molecule is a component of a human T-cell receptor (TCR) complex.
42. The combination of embodiment 41, wherein ABM1 is an antibody, an antibody fragment, an scFv, a dsFv, a Fv, a Fab, an scFab, a (Fab′)2, a single domain antibody (SDAB), a VH or VL domain, or a camelid VHH domain.
43. The combination of embodiment 42, wherein ABM1 is a Fab.
44. The combination of embodiment 43, wherein the Fab is a Fab heterodimer.
45. The combination of embodiment 42, wherein ABM1 is an scFv.
46. The combination of embodiment 42, wherein ABM1 is an anti-CD19 antibody or an antigen-binding domain thereof.
47. The combination of any one of embodiments 41 to 46, wherein ABM2 is a non-immunoglobulin scaffold based ABM.
48. The combination of embodiment 47, wherein ABM2 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.
49. The combination of any one of embodiments 41 to 46, wherein ABM2 is an immunoglobulin scaffold based ABM.
50. The combination of embodiment 49, wherein ABM2 is an antibody, an antibody fragment, an scFv, a dsFv, a Fv, a Fab, an scFab, a (Fab′)2, a single domain antibody (SDAB), a VH or VL domain, or a camelid VHH domain.
51. The combination of embodiment 50, wherein ABM2 is an antibody or an antigen-binding domain thereof.
52. The combination of embodiment 50, wherein ABM2 is an scFv.
53. The combination of embodiment 50, wherein ABM2 is a Fab.
54. The combination of embodiment 50, wherein ABM2 is a Fab heterodimer.
55. The combination of any one of embodiments 41 to 54, in which ABM2 binds specifically to a component of a human T-cell receptor (TCR) complex.
56. The combination of embodiment 55, wherein the component of the TCR complex is CD3.
57. The combination of embodiment 56, wherein ABM2 comprises the CDR sequences of CD3hi.
58. The combination of embodiment 56, wherein ABM2 comprises the CDR sequences of CD3med.
59. The combination of embodiment 56, wherein ABM2 comprises the CDR sequences of CD31o.
60. The combination of any one of embodiments 57 to 59, wherein the CDRs are defined by Kabat numbering.
61. The combination of any one of embodiments 57 to 59, wherein the CDRs are defined by Chothia numbering.
62. The combination of any one of embodiments 57 to 59, wherein the CDRs are defined by a combination of Kabat and Chothia numbering.
63. The combination of embodiment 56, wherein ABM2 comprises the heavy and light chain variable sequences of CD3hi, as set forth in Table 9A.
64. The combination of embodiment 56, wherein ABM2 comprises the heavy and light chain variable sequences of CD3med, as set forth in Table 9A.
65. The combination of embodiment 56, wherein ABM2 comprises the heavy and light chain variable sequences of CD31o, as set forth in Table 9A.
66. The combination of embodiment 55, wherein the component of the TCR complex is TCR-α, TCR-β, or a TCR-α/β dimer.
67. The combination of embodiment 66, wherein the component of the TCR complex is TCR-α.
68. The combination of embodiment 66, wherein the component of the TCR complex is TCR-R.
69. The combination of embodiment 66, wherein the component of the TCR complex is a TCR-α/β dimer.
70. The combination of embodiment 66, wherein ABM2 comprises the CDR sequences of BMA031.
71. The combination of embodiment 70, wherein the CDR sequences of BMA031 are defined by Kabat numbering.
72. The combination of embodiment 70, wherein the CDR sequences of BMA031 are defined by Chothia numbering.
73. The combination of embodiment 70, wherein the CDR sequences of BMA031 are defined by a combination of Kabat and Chothia numbering.
74. The combination of embodiment 70, wherein ABM2 comprises the heavy and light chain variable sequences of BMA031.
75. The combination of embodiment 55, wherein the component of the TCR complex is TCR-γ, TCR-δ, or a TCR-γ/δ dimer.
76. The combination of embodiment 75, wherein the component of the TCR complex is TCR-γ.
77. The combination of embodiment 75, wherein the component of the TCR complex is TCR-δ.
78. The combination of embodiment 75, wherein the component of the TCR complex is a TCR-γ/δ dimer.
79. The combination of embodiment 75, wherein ABM2 comprises the CDR sequences of δTCS1.
80. The combination of embodiment 79, wherein the CDR sequences of 6TCS1 are defined by Kabat numbering.
81. The combination of embodiment 79, wherein the CDR sequences of 6TCS1 are defined by Chothia numbering.
82. The combination of embodiment 79, wherein the CDR sequences of 6TCS1 are defined by a combination of Kabat and Chothia numbering.
83. The combination of embodiment 79, wherein ABM2 comprises the heavy and light chain variable sequences of 6TCS1.
84. The combination of any one of embodiments 41 to 83, in which ABM1 is capable of binding CD19 at the same time as ABM2 is bound to its target molecule.
85. The CD19 binding molecule of any one of embodiments 40 to 84, where the CD19 binding molecule is a bispecific binding molecule (BBM).
86. The combination of embodiment 85, wherein the BBM is bivalent.
87. The combination of embodiment 86, wherein the CD19 binding molecule has any one of the configurations depicted in FIGS. 1B-1F.
88. The combination of embodiment 87, wherein the CD19 binding molecule has the configuration depicted in FIG. 1B.
89. The combination of embodiment 87, wherein the CD19 binding molecule has the configuration depicted in FIG. 1C.
90. The combination of embodiment 87, wherein the CD19 binding molecule has the configuration depicted in FIG. 1D.
91. The combination of embodiment 87, wherein the CD19 binding molecule has the configuration depicted in FIG. 1E.
92. The combination of embodiment 87, wherein the CD19 binding molecule has the configuration depicted in FIG. 1F.
93. The combination of any one of embodiments 87 to 92, wherein the CD19 binding molecule has the configuration referred to as 1 in Section 7.2.3.1.
94. The combination of any one of embodiments 87 to 92, wherein the CD19 binding molecule has the configuration referred to as B2 in Section 7.2.3.1.
95. The combination of embodiment 85, wherein the CD19 binding molecule is trivalent.
96. The combination of embodiment 95, wherein the CD19 binding molecule has any one of the configurations depicted in FIGS. 1G-1Z.
97. The combination of embodiment 96, wherein the CD19 binding molecule has the configuration depicted in FIG. 1G.
98. The combination of embodiment 96, wherein the CD19 binding molecule has the configuration depicted in FIG. 1H.
99. The combination of embodiment 96, wherein the CD19 binding molecule has the configuration depicted in FIG. 11.
100. The combination of embodiment 96, wherein the CD19 binding molecule has the configuration depicted in FIG. 1J.
101. The combination of embodiment 96, wherein the CD19 binding molecule has the configuration depicted in FIG. 1K.
102. The combination of embodiment 96, wherein the CD19 binding molecule has the configuration depicted in FIG. 1L.
103. The combination of embodiment 96, wherein the CD19 binding molecule has the configuration depicted in FIG. 1M.
104. The combination of embodiment 96, wherein the CD19 binding molecule has the configuration depicted in FIG. 1N.
105. The combination of embodiment 96, wherein the CD19 binding molecule has the configuration depicted in FIG. 1O.
106. The combination of embodiment 96, wherein the CD19 binding molecule has the configuration depicted in FIG. 1P.
107. The combination of embodiment 96, wherein the CD19 binding molecule has the configuration depicted in FIG. 1Q.
108. The combination of embodiment 96, wherein the CD19 binding molecule has the configuration depicted in FIG. 1R.
109. The combination of embodiment 96, wherein the CD19 binding molecule has the configuration depicted in FIG. 1S.
110. The combination of embodiment 96, wherein the CD19 binding molecule has the configuration depicted in FIG. 1T.
111. The combination of embodiment 96, wherein the CD19 binding molecule has the configuration depicted in FIG. 1U.
112. The combination of embodiment 96, wherein the CD19 binding molecule has the configuration depicted in FIG. 1V.
113. The combination of embodiment 96, wherein the CD19 binding molecule has the configuration depicted in FIG. 1W.
114. The combination of embodiment 96, wherein the CD19 binding molecule has the configuration depicted in FIG. 1X.
115. The combination of embodiment 96, wherein the CD19 binding molecule has the configuration depicted in FIG. 1Y.
116. The combination of embodiment 96, wherein the CD19 binding molecule has the configuration depicted in FIG. 1Z.
117. The combination of any one of embodiments 95 to 116, wherein the CD19 binding molecule has the configuration referred to as T1 in Section 7.2.3.2.
118. The combination of any one of embodiments 95 to 116, wherein the CD19 binding molecule has the configuration referred to as T2 in Section 7.2.3.2.
119. The combination of any one of embodiments 95 to 116, wherein the CD19 binding molecule has the configuration referred to as T3 in Section 7.2.3.2.
120. The combination of any one of embodiments 95 to 116, wherein the CD19 binding molecule has the configuration referred to as T4 in Section 7.2.3.2.
121. The combination of any one of embodiments 95 to 116, wherein the CD19 binding molecule has the configuration referred to as T5 in Section 7.2.3.2.
122. The combination of any one of embodiments 95 to 116, wherein the CD19 binding molecule has the configuration referred to as T6 in Section 7.2.3.2.
123. The combination of embodiment 85, wherein the CD19 binding molecule is tetravalent.
124. The combination of embodiment 123, wherein the CD19 binding molecule has any one of the configurations depicted in FIGS. 1AA-1AH.
125. The combination of embodiment 124, wherein the CD19 binding molecule has the configuration depicted in FIG. 1AA.
126. The combination of embodiment 124, wherein the CD19 binding molecule has the configuration depicted in FIG. 1AB.
127. The combination of embodiment 124, wherein the CD19 binding molecule has the configuration depicted in FIG. 1AC.
128. The combination of embodiment 124, wherein the CD19 binding molecule has the configuration depicted in FIG. 1AD.
129. The combination of embodiment 124, wherein the CD19 binding molecule has the configuration depicted in FIG. 1AE.
130. The combination of embodiment 124, wherein the CD19 binding molecule has the configuration depicted in FIG. 1AF.
131. The combination of embodiment 124, wherein the CD19 binding molecule has the configuration depicted in FIG. 1AG.
132. The combination of embodiment 124, wherein the CD19 binding molecule has the configuration depicted in FIG. 1AH.
133. The combination of any one of embodiments 123 to 132, wherein the CD19 binding molecule has the configuration referred to as Tv 1 in Section 7.2.3.3.
134. The combination of any one of embodiments 123 to 132, wherein the CD19 binding molecule has the configuration referred to as Tv 2 in Section 7.2.3.3.
135. The combination of any one of embodiments 123 to 132, wherein the CD19 binding molecule has the configuration referred to as Tv 3 in Section 7.2.3.3.
136. The combination of any one of embodiments 123 to 132, wherein the CD19 binding molecule has the configuration referred to as Tv 4 in Section 7.2.3.3.
137. The combination of any one of embodiments 123 to 132, wherein the CD19 binding molecule has the configuration referred to as Tv 5 in Section 7.2.3.3.
138. The combination of any one of embodiments 123 to 132, wherein the CD19 binding molecule has the configuration referred to as Tv 6 in Section 7.2.3.3.
139. The combination of any one of embodiments 123 to 132, wherein the CD19 binding molecule has the configuration referred to as Tv 7 in Section 7.2.3.3.
140. The combination of any one of embodiments 123 to 132, wherein the CD19 binding molecule has the configuration referred to as Tv 8 in Section 7.2.3.3.
141. The combination of any one of embodiments 123 to 132, wherein the CD19 binding molecule has the configuration referred to as Tv 9 in Section 7.2.3.3.
142. The combination of any one of embodiments 123 to 132, wherein the CD19 binding molecule has the configuration referred to as Tv 10 in Section 7.2.3.3.
143. The combination of any one of embodiments 123 to 132, wherein the CD19 binding molecule has the configuration referred to as Tv 11 in Section 7.2.3.3.
144. The combination of any one of embodiments 123 to 132, wherein the CD19 binding molecule has the configuration referred to as Tv 12 in Section 7.2.3.3.
145. The combination of any one of embodiments 123 to 132, wherein the CD19 binding molecule has the configuration referred to as Tv 13 in Section 7.2.3.3.
146. The combination of any one of embodiments 123 to 132, wherein the CD19 binding molecule has the configuration referred to as Tv 14 in Section 7.2.3.3.
147. The combination of any one of embodiments 41 to 84, wherein the CD19 binding molecule is a trispecific binding molecule (TBM) comprising an antigen-binding module 3 (ABM3) that binds specifically to a target molecule other than CD19.
148. The combination of embodiment 147, in which ABM2 binds specifically to a component of a human T-cell receptor (TCR) complex and ABM3 binds specifically to (i) human CD2 or (ii) a tumor associated antigen (TAA).
149. The combination of embodiment 147 or embodiment 148, which is trivalent.
150. The combination of embodiment 149, wherein the CD19 binding molecule has any one of the configurations depicted in FIGS. 2A-2P.
151. The combination of embodiment 150, wherein the CD19 binding molecule has the configuration depicted in FIG. 2A.
152. The combination of embodiment 150, wherein the CD19 binding molecule has the configuration depicted in FIG. 2B.
153. The combination of embodiment 150, wherein the CD19 binding molecule has the configuration depicted in FIG. 2C.
154. The combination of embodiment 150, wherein the CD19 binding molecule has the configuration depicted in FIG. 2D.
155. The combination of embodiment 150, wherein the CD19 binding molecule has the configuration depicted in FIG. 2E.
156. The combination of embodiment 150, wherein the CD19 binding molecule has the configuration depicted in FIG. 2F.
157. The combination of embodiment 150, wherein the CD19 binding molecule has the configuration depicted in FIG. 2G.
158. The combination of embodiment 150, wherein the CD19 binding molecule has the configuration depicted in FIG. 2H.
159. The combination of embodiment 150, wherein the CD19 binding molecule has the configuration depicted in FIG. 2I.
160. The combination of embodiment 150, wherein the CD19 binding molecule has the configuration depicted in FIG. 2J.
161. The combination of embodiment 150, wherein the CD19 binding molecule has the configuration depicted in FIG. 2K.
162. The combination of embodiment 150, wherein the CD19 binding molecule has the configuration depicted in FIG. 2L.
163. The combination of embodiment 150, wherein the CD19 binding molecule has the configuration depicted in FIG. 2M.
164. The combination of embodiment 150, wherein the CD19 binding molecule has the configuration depicted in FIG. 2N.
165. The combination of embodiment 150, wherein the CD19 binding molecule has the configuration depicted in FIG. 2O.
166. The combination of embodiment 150, wherein the CD19 binding molecule has the configuration depicted in FIG. 2P.
167. The combination of any one of embodiments 150 to 166, wherein the CD19 binding molecule has the configuration referred to as T1 in Section 7.2.4.1.
168. The combination of any one of embodiments 150 to 166, wherein the CD19 binding molecule has the configuration referred to as T2 in Section 7.2.4.1.
169. The combination of any one of embodiments 150 to 166, wherein the CD19 binding molecule has the configuration referred to as T3 in Section 7.2.4.1.
170. The combination of any one of embodiments 150 to 166, wherein the CD19 binding molecule has the configuration referred to as T4 in Section 7.2.4.1.
171. The combination of any one of embodiments 150 to 166, wherein the CD19 binding molecule has the configuration referred to as T5 in Section 7.2.4.1.
172. The combination of any one of embodiments 150 to 166, wherein the CD19 binding molecule has the configuration referred to as T6 in Section 7.2.4.1.
173. The combination of embodiment 147 or embodiment 148, wherein the CD19 binding molecule is tetravalent.
174. The combination of embodiment 173, wherein the CD19 binding molecule has any one of the configurations depicted in FIGS. 2Q-2S.
175. The combination of embodiment 174, wherein the CD19 binding molecule has the configuration depicted in FIG. 2Q.
176. The combination of embodiment 174, wherein the CD19 binding molecule has the configuration depicted in FIG. 2R.
177. The combination of embodiment 174, wherein the CD19 binding molecule has the configuration depicted in FIG. 2S.
178. The combination of embodiment 147 or embodiment 148, wherein the CD19 binding molecule is pentavalent.
179. The combination of embodiment 178, wherein the CD19 binding molecule has the configuration depicted in FIG. 2T.
180. The combination of embodiment 147 or embodiment 148, wherein the CD19 binding molecule is hexavalent.
181. The combination of embodiment 180, wherein the CD19 binding molecule has any one of the configurations depicted in FIGS. 2U-2V.
182. The combination of embodiment 181, wherein the CD19 binding molecule has the configuration depicted in FIG. 2U.
183. The combination of embodiment 181, wherein the CD19 binding molecule has the configuration depicted in FIG. 2V.
184. The combination of any one of embodiments 147 to 183, in which ABM1 is capable of binding CD19 at the same time ABM3 is bound to its target molecule.
185. The combination of any one of embodiments 147 to 184, in which ABM3 binds specifically to human CD2.
186. The combination of embodiment 185, wherein ABM3 is a non-immunoglobulin scaffold based ABM.
187. The combination of embodiment 186, wherein ABM3 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.
188. The combination of embodiment 186, wherein ABM3 comprises a receptor binding domain of a CD2 ligand.
189. The combination of embodiment 185, wherein ABM3 is a CD58 moiety.
190. The combination of embodiment 189, wherein the CD58 moiety comprises the amino acid sequence of CD58-1 as set forth in Table 12.
191. The combination of embodiment 189, wherein the CD58 moiety comprises the amino acid sequence of CD58-2 as set forth in Table 12.
192. The combination of embodiment 189, wherein the CD58 moiety comprises the amino acid sequence of CD58-3 as set forth in Table 12.
193. The combination of embodiment 189, wherein the CD58 moiety comprises the amino acid sequence of CD58-4 as set forth in Table 12.
194. The combination of embodiment 189, wherein the CD58 moiety comprises the amino acid sequence of CD58-5 as set forth in Table 12.
195. The combination of embodiment 194, wherein the amino acid designated as B is a phenylalanine.
196. The combination of embodiment 194, wherein the amino acid designated as B is a serine.
197. The combination of any one of embodiments 194 to 196, wherein the amino acid designated as J is a valine.
198. The combination of any one of embodiments 194 to 196, wherein the amino acid designated as J is a lysine.
199. The combination of any one of embodiments 194 to 198, wherein the amino acid designated as O is a valine.
200. The combination of any one of embodiments 194 to 198, wherein the amino acid designated as O is a glutamine.
201. The combination of any one of embodiments 194 to 200, wherein the amino acid designated as U is a valine.
202. The combination of any one of embodiments 194 to 200, wherein the amino acid designated as U is a lysine.
203. The combination of any one of embodiments 194 to 202, wherein the amino acid designated as X is a threonine.
204. The combination of any one of embodiments 194 to 202, wherein the amino acid designated as X is a serine.
205. The combination of any one of embodiments 194 to 204, wherein the amino acid designated as Z is a leucine.
206. The combination of any one of embodiments 194 to 204, wherein the amino acid designated as Z is a glycine.
207. The combination of embodiment 189, wherein the CD58 moiety comprises the amino acid sequence of CD58-6 as set forth in Table 12.
208. The combination of embodiment 189, wherein the CD58 moiety comprises the amino acid sequence of CD58-7 as set forth in Table 12.
209. The combination of embodiment 208, wherein the amino acid designated as J is a valine.
210. The combination of embodiment 208, wherein the amino acid designated as J is a lysine.
211. The combination of any one of embodiments 208 to 210, wherein the amino acid designated as O is a valine.
212. The combination of any one of embodiments 208 to 210, wherein the amino acid designated as O is a glutamine.
213. The combination of embodiment 189, wherein the CD58 moiety comprises the amino acid sequence of CD58-8 as set forth in Table 12.
214. The combination of embodiment 189, wherein the CD58 moiety comprises the amino acid sequence of CD58-9 as set forth in Table 12.
215. The combination of embodiment 189, wherein the CD58 moiety comprises the amino acid sequence of CD58-10 as set forth in Table 12.
216. The combination of embodiment 189, wherein the CD58 moiety comprises the amino acid sequence of CD58-11 as set forth in Table 12.
217. The combination of embodiment 185, wherein ABM3 is a CD48 moiety.
218. The combination of embodiment 217, wherein the CD48 moiety has at least 70% sequence identity to amino acids 27-220 of the amino acid sequence of Uniprot identifier P09326.
219. The combination of embodiment 217, wherein the CD48 moiety has at least 80% sequence identity to amino acids 27-220 of the amino acid sequence of Uniprot identifier P09326.
220. The combination of embodiment 217, wherein the CD48 moiety has at least 90% sequence identity to amino acids 27-220 of the amino acid sequence of Uniprot identifier P09326.
221. The combination of embodiment 217, wherein the CD48 moiety has at least 95% sequence identity to amino acids 27-220 of the amino acid sequence of Uniprot identifier P09326.
222. The combination of embodiment 217, wherein the CD48 moiety has at least 99% sequence identity to amino acids 27-220 of the amino acid sequence of Uniprot identifier P09326.
223. The combination of embodiment 185, wherein ABM3 is an immunoglobulin scaffold based ABM.
224. The combination of embodiment 223, wherein ABM3 is an antibody, an antibody fragment, an scFv, a dsFv, a Fv, a Fab, an scFab, a (Fab′)2, a single domain antibody (SDAB), a VH or VL domain, or a camelid VHH domain.
225. The combination of embodiment 223, wherein ABM3 is an anti-CD2 antibody or an antigen-binding domain thereof.
226. The combination of embodiment 224, wherein ABM3 is an scFv.
227. The combination of embodiment 224, wherein ABM3 is a Fab.
228. The combination of embodiment 227, wherein ABM3 is a Fab heterodimer.
229. The combination of any one of embodiments 223 to 228, wherein ABM3 comprises the CDR sequences of CD2-1.
230. The combination of embodiment 229, wherein ABM3 comprises the heavy and light chain variable sequences of CD2-1.
231. The combination of embodiment 229, wherein ABM3 comprises the heavy and light chain variable sequences of hu1CD2-1.
232. The combination of embodiment 229, wherein ABM3 comprises the heavy and light chain variable sequences of hu2CD2-1.
233. The combination of embodiment 229, wherein ABM3 comprises the CDR sequences of Medi 507.
234. The combination of embodiment 233, wherein ABM3 comprises the heavy and light chain variable sequences of Medi 507.
235. The combination of any one of embodiments 147 to 184, in which ABM3 binds specifically to a human TAA.
236. The combination of embodiment 235, wherein ABM3 is a non-immunoglobulin scaffold based ABM.
237. The combination of embodiment 236, wherein if TAA is a receptor, ABM3 comprises a receptor binding domain of a ligand of the receptor, and if TAA is a ligand, ABM3 comprises a ligand binding domain of a receptor of the ligand.
238. The combination of embodiment 236, wherein ABM3 is a Kunitz domain, an Adnexin, an Affibody, a DARPin, an Avimer, an Anticalin, a Lipocalin, a Centyrin, a Versabody, a Knottin, an Adnectin, a Pronectin, an Affitin/Nanofitin, an Affilin, an Atrimer/Tetranectin, a bicyclic peptide, a cys-knot, a Fn3 scaffold, an Obody, a Tn3, an Affimer, BD, an Adhiron, a Duocalin, an Alphabody, an Armadillo Repeat Protein, a Repebody, or a Fynomer.
239. The combination of embodiment 235, wherein ABM3 is an immunoglobulin scaffold based ABM.
240. The combination of embodiment 239, wherein ABM3 is an antibody, an antibody fragment, an scFv, a dsFv, a Fv, a Fab, an scFab, a (Fab′)2, a single domain antibody (SDAB), a VH or VL domain, or a camelid VHH domain.
241. The combination of embodiment 240, wherein ABM3 is an antibody or an antigen-binding domain thereof.
242. The combination of embodiment 240, wherein ABM3 is an scFv.
243. The combination of embodiment 240, wherein ABM3 is a Fab.
244. The combination of embodiment 243, wherein ABM3 is a Fab heterodimer.
245. The combination of any one of embodiments 235 to 244, wherein the TAA is a TAA expressed on cancerous B cells that are B cell-derived plasma cells.
246. The combination of any one of embodiments 235 to 245, wherein the TAA is a TAA expressed on cancerous B cells that are not plasma cells.
247. The combination of any one of embodiments 235 to 246, wherein the TAA is selected from BCMA, CD20, CD22, CD123, CD33, CLL1, CD138, CS1, CD38, CD133, FLT3, CD52, TNFRSF13C, TNFRSF13B, CXCR4, PD-L1, LY9, CD200, FCGR2B, CD21, CD23, CD24, CD40L, CD72, CD79a, and CD79b.
248. The combination of embodiment 247, wherein the TAA is BCMA.
249. The combination of embodiment 247, wherein the TAA is CD20.
250. The combination of embodiment 247, wherein the TAA is CD22.
251. The combination of embodiment 247, wherein the TAA is CD123.
252. The combination of embodiment 247, wherein the TAA is CD33.
253. The combination of embodiment 247, wherein the TAA is CLL1.
254. The combination of embodiment 247, wherein the TAA is CD138.
255. The combination of embodiment 247, wherein the TAA is CS1.
256. The combination of embodiment 247, wherein the TAA is CD38.
257. The combination of embodiment 247, wherein the TAA is CD133.
258. The combination of embodiment 247, wherein the TAA is FLT3.
259. The combination of embodiment 247, wherein the TAA is CD52.
260. The combination of embodiment 247, wherein the TAA is TNFRSF13C.
261. The combination of embodiment 247, wherein the TAA is TNFRSF13B.
262. The combination of embodiment 247, wherein the TAA is CXCR4.
263. The combination of embodiment 247, wherein the TAA is PD-L1.
264. The combination of embodiment 247, wherein the TAA is LY9.
265. The combination of embodiment 247, wherein the TAA is CD200.
266. The combination of embodiment 247, wherein the TAA is FCGR2B.
267. The combination of embodiment 247, wherein the TAA is CD21.
268. The combination of embodiment 247, wherein the TAA is CD23.
269. The combination of embodiment 247, wherein ABM3 comprises a binding sequence set forth in Table 13.
270. The combination of any one of embodiments 38 to 269, wherein the CD19 binding molecule comprises a first variant Fc region and a second variant Fc region forming a Fc domain.
271. The combination of embodiment 270, wherein the first variant Fc region is a variant human IgG1 Fc region and the second variang Fc region is a human IgG1 Fc region, wherein the first and second variant Fc regions comprise L234A, L235A, and G237A (“LALAGA”) substitutions, L234A, L235A, S267K, and P329A (“LALASKPA”) substitutions, D265A, P329A, and S267K (“DAPASK”) substitutions, G237A, D265A, and P329A (“GADAPA”) substitutions, G237A, D265A, P329A, and S267K (“GADAPASK”) substitutions, L234A, L235A, and P329G (“LALAPG”) substitutions, or L234A, L235A, and P329A (“LALAPA”) substitutions, wherein the amino acid residues are numbered according to the EU numbering system.
272. The combination of embodiment 271, wherein the first variant Fc region and the second variant Fc region comprise L234A, L235A, and G237A (“LALAGA”) substitutions, wherein the amino acid residues are numbered according to the EU numbering system.
273. The combination of embodiment 271, wherein the first variant Fc region and the second variant Fc region comprise L234A, L235A, S267K, and P329A (“LALASKPA”) substitutions, wherein the amino acid residues are numbered according to the EU numbering system.
274. The combination of embodiment 271, wherein the first variant Fc region and the second variant Fc region comprise D265A, P329A, and S267K (“DAPASK”) substitutions, wherein the amino acid residues are numbered according to the EU numbering system.
275. The combination of embodiment 271, the first variant Fc region and the second variant Fc region comprise G237A, D265A, and P329A (“GADAPA”) substitutions, wherein the amino acid residues are numbered according to the EU numbering system.
276. The combination of embodiment 271, wherein the first variant Fc region and the second variant Fc region comprise G237A, D265A, P329A, and S267K (“GADAPASK”) substitutions, wherein the amino acid residues are numbered according to the EU numbering system.
277. The combination of embodiment 271, wherein the first variant Fc region and the second variant Fc region comprise L234A, L235A, and P329G (“LALAPG”) substitutions, wherein the amino acid residues are numbered according to the EU numbering system.
278. The combination of embodiment 271, wherein the first variant Fc region and the second variant Fc region comprise L234A, L235A, and P329A (“LALAPA”) substitutions, wherein the amino acid residues are numbered according to the EU numbering system.
279. The combination of embodiment 271, wherein the first variant Fc region and/or the second variant Fc region comprises a sequence having at least 95% identity to FCV-1, FCV-2, FCV-3, FCV-4, FCV-5, FCV-6, or FCV-7.
280. The combination of embodiment 279, wherein the first variant Fc region and/or the second variant Fc region comprises a sequence having at least 95% identity to FCV-1.
281. The combination of embodiment 279, wherein the first variant Fc region and/or the second variant Fc region comprises a sequence having at least 96% identity to FCV-1.
282. The combination of embodiment 279, wherein the first variant Fc region and/or the second variant Fc region comprises a sequence having at least 97% identity to FCV-1.
283. The combination of embodiment 279, wherein the first variant Fc region and/or the second variant Fc region comprises a sequence having at least 98% identity to FCV-1.
284. The combination of embodiment 279, wherein the first variant Fc region and/or the second variant Fc region comprises a sequence having at least 99% identity to FCV-1.
285. The combination of embodiment 279, wherein the first variant Fc region and/or the second variant Fc region comprises a sequence having at least 95% identity to FCV-2.
286. The combination of embodiment 279, wherein the first variant Fc region and/or the second variant Fc region comprises a sequence having at least 96% identity to FCV-2.
287. The combination of embodiment 279, wherein the first variant Fc region and/or the second variant Fc region comprises a sequence having at least 97% identity to FCV-2.
288. The combination of embodiment 279, wherein the first variant Fc region and/or the second variant Fc region comprises a sequence having at least 98% identity to FCV-2.
289. The combination of embodiment 279, wherein the first variant Fc region and/or the second variant Fc region comprises a sequence having at least 99% identity to FCV-2.
290. The combination of embodiment 279, wherein the first variant Fc region and/or the second variant Fc region comprises a sequence having at least 95% identity to FCV-3.
291. The combination of embodiment 279, wherein the first variant Fc region and/or the second variant Fc region comprises a sequence having at least 96% identity to FCV-3.
292. The combination of embodiment 279, wherein the first variant Fc region and/or the second variant Fc region comprises a sequence having at least 97% identity to FCV-3.
293. The combination of embodiment 279, wherein the first variant Fc region and/or the second variant Fc region comprises a sequence having at least 98% identity to FCV-3.
294. The combination of embodiment 279, wherein the first variant Fc region and/or the second variant Fc region comprises a sequence having at least 99% identity to FCV-3.
295. The combination of embodiment 279, wherein the first variant Fc region and/or the second variant Fc region comprises a sequence having at least 95% identity to FCV-4.
296. The combination of embodiment 279, wherein the first variant Fc region and/or the second variant Fc region comprises a sequence having at least 96% identity to FCV-4.
297. The combination of embodiment 279, wherein the first variant Fc region and/or the second variant Fc region comprises a sequence having at least 97% identity to FCV-4.
298. The combination of embodiment 279, wherein the first variant Fc region and/or the second variant Fc region comprises a sequence having at least 98% identity to FCV-4.
299. The combination of embodiment 279, wherein the first variant Fc region and/or the second variant Fc region comprises a sequence having at least 99% identity to FCV-4.
300. The combination of embodiment 279, wherein the first variant Fc region and/or the second variant Fc region comprises a sequence having at least 95% identity to FCV-5.
301. The combination of embodiment 279, wherein the first variant Fc region and/or the second variant Fc region comprises a sequence having at least 96% identity to FCV-5.
302. The combination of embodiment 279, wherein the first variant Fc region and/or the second variant Fc region comprises a sequence having at least 97% identity to FCV-5.
303. The combination of embodiment 279, wherein the first variant Fc region and/or the second variant Fc region comprises a sequence having at least 98% identity to FCV-5.
304. The combination of embodiment 279, wherein the first variant Fc region and/or the second variant Fc region comprises a sequence having at least 99% identity to FCV-5.
305. The combination of embodiment 279, wherein the first variant Fc region and/or the second variant Fc region comprises a sequence having at least 95% identity to FCV-6.
306. The combination of embodiment 279, wherein the first variant Fc region and/or the second variant Fc region comprises a sequence having at least 96% identity to FCV-6.
307. The combination of embodiment 279, wherein the first variant Fc region and/or the second variant Fc region comprises a sequence having at least 97% identity to FCV-6.
308. The combination of embodiment 279, wherein the first variant Fc region and/or the second variant Fc region comprises a sequence having at least 98% identity to FCV-6.
309. The combination of embodiment 279, wherein the first variant Fc region and/or the second variant Fc region comprises a sequence having at least 99% identity to FCV-6.
310. The combination of embodiment 279, wherein the first variant Fc region and/or the second variant Fc region comprises a sequence having at least 95% identity to FCV-7.
311. The combination of embodiment 279, wherein the first variant Fc region and/or the second variant Fc region comprises a sequence having at least 96% identity to FCV-7.
312. The combination of embodiment 279, wherein the first variant Fc region and/or the second variant Fc region comprises a sequence having at least 97% identity to FCV-7.
313. The combination of embodiment 279, wherein the first variant Fc region and/or the second variant Fc region comprises a sequence having at least 98% identity to FCV-7.
314. The combination of embodiment 279, wherein the first variant Fc region and/or the second variant Fc region comprises a sequence having at least 99% identity to FCV-7.
315. The combination of embodiment 279, wherein the first variant Fc region and/or the second variant Fc region comprises a sequence having 100% identity to FCV-1.
316. The combination of embodiment 279, wherein the first variant Fc region and/or the second variant Fc region comprises a sequence having 100% identity to FCV-2.
317. The combination of embodiment 279, wherein the first variant Fc region and/or the second variant Fc region comprises a sequence having 100% identity to FCV-3.
318. The combination of embodiment 279, wherein the first variant Fc region and/or the second variant Fc region comprises a sequence having 100% identity to FCV-4.
319. The combination of embodiment 279, wherein the first variant Fc region and/or the second variant Fc region comprises a sequence having 100% identity to FCV-5.
320. The combination of embodiment 279, wherein the first variant Fc region and/or the second variant Fc region comprises a sequence having 100% identity to FCV-6.
321. The combination of embodiment 279, wherein the first variant Fc region and/or the second variant Fc region comprises a sequence having 100% identity to FCV-7.
322. The combination of any one of embodiments 270 to 321, wherein the first variant Fc region and the second variant Fc region together form an Fc heterodimer.
323. The combination of embodiment 322, wherein the first and second variant Fc regions comprise the amino acid substitutions amino acid substitutions T366W: T366S/L368A/Y407V.
324. The combination of embodiment 322, wherein the first and second variant Fc regions comprise the amino acid substitutions S364K/E357Q:L368D/K370S.
325. The combination of embodiments 322, wherein the first and second variant Fc regions comprise the amino acid substitutions L368D/K370S:S364K.
326. The combination of embodiment 322, wherein the first and second variant Fc regions comprise the amino acid substitutions L368E/K370S:S364K.
327. The combination of embodiment 322, wherein the first and second variant Fc regions comprise the amino acid substitutionsT411T/E360E/Q362E: D401K.
328. The combination of embodiment 322, wherein the first and second variant Fc regions comprise the amino acid substitutions L368D 370S: S364/E357L.
329. The combination of embodiment 322, wherein the first and second variant Fc regions comprise the amino acid substitutions 370S: S364K/E357Q.
330. The combination of embodiment 322, wherein the first and second variant Fc regions comprise the amino acid substitutions of any one of the steric variants listed in FIG. 4 of WO 2014/110601 (reproduced in Table 4).
331. The combination of embodiment 322, wherein the first and second variant Fc regions comprise the amino acid substitutions of any one of the variants listed in FIG. 5 of WO 2014/110601 (reproduced in Table 4).
332. The combination of embodiment 322, wherein the first and second variant Fc regions comprise the amino acid substitutions of any one of the variants listed in FIG. 6 of WO 2014/110601 (reproduced in Table 4).
333. The combination of any one of embodiments 322 to 332, wherein at least one of the Fc regions comprises an ablation variant modification.
334. The combination of embodiment 333, wherein the ablation variant modifications are selected from Table 3.
335. The combination of embodiment 334, wherein the ablation variant modification comprises G236R.
336. The combination of embodiment 334, wherein the ablation variant modification comprises S239G.
337. The combination of embodiment 334, wherein the ablation variant modification comprises S239K.
338. The combination of embodiment 334, wherein the ablation variant modification comprises S239Q.
339. The combination of embodiment 334, wherein the ablation variant modification comprises S239R.
340. The combination of embodiment 334, wherein the ablation variant modification comprises V266D.
341. The combination of embodiment 334, wherein the ablation variant modification comprises S267K.
342. The combination of embodiment 334, wherein the ablation variant modification comprises S267R.
343. The combination of embodiment 334, wherein the ablation variant modification comprises H268K.
344. The combination of embodiment 334, wherein the ablation variant modification comprises E269R.
345. The combination of embodiment 334, wherein the ablation variant modification comprises 299R.
346. The combination of embodiment 334, wherein the ablation variant modification comprises 299K.
347. The combination of embodiment 334, wherein the ablation variant modification comprises K322A.
348. The combination of embodiment 334, wherein the ablation variant modification comprises A327G.
349. The combination of embodiment 334, wherein the ablation variant modification comprises A327L.
350. The combination of embodiment 334, wherein the ablation variant modification comprises A327N.
351. The combination of embodiment 334, wherein the ablation variant modification comprises A327Q.
352. The combination of embodiment 334, wherein the ablation variant modification comprises L328E.
353. The combination of embodiment 334, wherein the ablation variant modification comprises L328R.
354. The combination of embodiment 334, wherein the ablation variant modification comprises P329A.
355. The combination of embodiment 334, wherein the ablation variant modification comprises P329H.
356. The combination of embodiment 334, wherein the ablation variant modification comprises P329K.
357. The combination of embodiment 334, wherein the ablation variant modification comprises A330L.
358. The combination of embodiment 334, wherein the ablation variant modification comprises A330S/P331S.
359. The combination of embodiment 334, wherein the ablation variant modification comprises 1332K.
360. The combination of embodiment 334, wherein the ablation variant modification comprises 1332R.
361. The combination of embodiment 334, wherein the ablation variant modification comprises V266D/A327Q.
362. The combination of embodiment 334, wherein the ablation variant modification comprises V266D/P329K.
363. The combination of embodiment 334, wherein the ablation variant modification comprises G236R/L328R.
364. The combination of embodiment 334, wherein the ablation variant modification comprises E233P/L234V/L235A/G236del/S239K.
365. The combination of embodiment 334, wherein the ablation variant modification comprises E233P/L234V/L235A/G236del/S267K.
366. The combination of embodiment 334, wherein the ablation variant modification comprises E233P/L234V/L235A/G236del/S239K/A327G.
367. The combination of embodiment 334, wherein the ablation variant modification comprises E233P/L234V/L235A/G236del/S267K/A327G.
368. The combination of embodiment 334, wherein the ablation variant modification comprises E233P/L234V/L235A/G236del.
369. The combination of embodiment 334, wherein the ablation variant modification comprises S239K/S267K.
370. The combination of embodiment 334, wherein the ablation variant modification comprises 267K/P329K.
371. The combination of embodiment 334, wherein the ablation variant modification comprises D265A/N297A/P329A.
372. The combination of embodiment 334, wherein the ablation variant modification comprises D265N/N297D/P329G.
373. The combination of embodiment 334, wherein the ablation variant modification comprises D265E/N297Q/P329S.
374. The combination of any one of embodiments 333 to 373, wherein both variant Fc regions comprise the ablation variant modification.
375. The combination of any one of embodiments 322 to 374, wherein at least one of the Fc regions further comprises pl variant substitutions.
376. The combination of embodiment 375 wherein the pl variant substitutions are selected from Table 4.
377. The combination of embodiment 376, wherein the pl variant substitutions comprise the substitutions present in pl_ISO(−).
378. The combination of embodiment 376, wherein the pl variant substitutions comprise the substitutions present in pl_(−)_isosteric_A.
379. The combination of embodiment 376, wherein the pl variant substitutions comprise the substitutions present in pl_(−)_isosteric_B.
380. The combination of embodiment 376, wherein the pl variant substitutions comprise the substitutions present in Pl_ISO(+RR).
381. The combination of embodiment 376, wherein the pl variant substitutions comprise the substitutions present in pl_ISO(+).
382. The combination of embodiment 376, wherein the pl variant substitutions comprise the substitutions present in pl_(+)_isosteric_A.
383. The combination of embodiment 376, wherein the pl variant substitutions comprise the substitutions present in pl_(+)_isosteric_B.
384. The combination of embodiment 376, wherein the pl variant substitutions comprise the substitutions present in pl_(+)_isosteric_E269Q/E272Q.
385. The combination of embodiment 376, wherein the pl variant substitutions comprise the substitutions present in pl_(+)_isosteric_E269Q/E283Q.
386. The combination of embodiment 376, wherein the pl variant substitutions comprise the substitutions present in pl_(+)_isosteric_E2720/E283Q.
387. The combination of embodiment 376, wherein the pl variant substitutions comprise the substitutions present in pl_(+)_isosteric_E269Q.
388. The combination of any one of embodiments 322 to 387, wherein the first and/or second Fc region further comprises one or more amino acid substitution(s) selected from 434A, 434S, 428L, 308F, 2591, 428L/434S, 2591/308F, 4361/428L, 4361 or V/434S, 436V/428L, 252Y, 252Y/254T/256E, 2591/308F/428L, 236A, 239D, 239E, 332E, 332D, 239D/332E, 267D, 267E, 328F, 267E/328F, 236A/332E, 239D/332E/330Y, 239D, 332E/330L, 236R, 328R, 236R/328R, 236N/267E, 243L, 298A and 299T.
389. The combination of any one of embodiments 322 to 387, wherein the first and/or second Fc region further comprises the amino acid substitution 434A, 434S or 434V.
390. The combination of embodiment 389, wherein the first and/or second Fc region further comprises the amino acid substitution 428L.
391. The combination of any one of embodiments 389 to 390, wherein the first and/or second Fc region further the amino acid substitution 308F.
392. The combination of any one of embodiments 389 to 391, wherein the first and/or second Fc region further comprises the amino acid substitution 2591.
393. The combination of any one of embodiments 389 to 392, wherein the first and/or second Fc region further comprises the amino acid substitution 4361.
394. The combination of any one of embodiments 389 to 393, wherein the first and/or second Fc region further comprises the amino acid substitution 252Y.
395. The combination of any one of embodiments 389 to 394, wherein the first and/or second Fc region further comprises the amino acid substitution 254T.
396. The combination of any one of embodiments 389 to 395, wherein the first and/or second Fc region further comprises the amino acid substitution 256E.
397. The combination of any one of embodiments 389 to 396, wherein the first and/or second Fc region further comprises the amino acid substitution 239D or 239E.
398. The combination of any one of embodiments 389 to 397, wherein the first and/or second Fc region further comprises the amino acid substitution 332E or 332D.
399. The combination of any one of embodiments 389 to 398, wherein the first and/or second Fc region further comprises the amino acid substitution 267D or 267E.
400. The combination of any one of embodiments 389 to 399, wherein the first and/or second Fc region further comprises the amino acid substitution 330L.
401. The combination of any one of embodiments 389 to 400, wherein the first and/or second Fc region further comprises the amino acid substitution 236R or 236N.
402. The combination of any one of embodiments 389 to 401, wherein the first and/or second Fc region further comprises the amino acid substitution 328R.
403. The combination of any one of embodiments 389 to 402, wherein the first and/or second Fc region further comprises the amino acid substitution 243L.
404. The combination of any one of embodiments 389 to 403, wherein the first and/or second Fc region further comprises the amino acid substitution 298A.
405. The combination of any one of embodiments 389 to 404, wherein the first and/or second Fc region further comprises the amino acid substitution 299T.
406. The combination of embodiment 322, wherein:
   (a) the first and second variant Fc regions comprise the amino acid substitutions S364K/E357Q: L368D/K370S;
   (b) the first and/or second variant Fc regions comprises the ablation variant modifications E233P/L234V/L235A/G236del/S267K, and
   (c) the first and/or second variant Fc regions comprises the pl variant substitutions N208D/Q295E/N384D/Q418E/N421D (pl_(−)_isosteric_A).
407. The combination of embodiment 406, wherein the first variant Fc region comprises the ablation variant modifications E233P/L234V/L235A/G236del/S267K.
408. The combination of any one of embodiments 406 to 407, wherein the second variant Fc region comprises the ablation variant modifications E233P/L234V/L235A/G236del/S267K.
409. The combination of any one of embodiments 406 to 408, wherein the first variant Fc region comprises the pl variant substitutions N208D/Q295E/N384D/Q418E/N421D (pl_(−)_isosteric_A).
410. The combination of any one of embodiments 406 to 409, wherein the second variant Fc region comprises the pl variant substitutions N208D/Q295E/N384D/Q418E/N421D (pl_(−)_isosteric_A).
411. The combination of any one of embodiments 322 to 410, wherein the first or second variant Fc region comprises an amino acid sequence which is at least 90% identical to SEQ ID NO:252.
412. The combination of any one of embodiments 322 to 410, wherein the first or second variant Fc region comprises an amino acid sequence which is at least 95% identical to SEQ ID NO:252.
413. The combination of any one of embodiments 322 to 410, wherein the first or second variant Fc region comprises the amino acid sequence of SEQ ID NO:252 modified with the substitutions recited in any one of embodiments 324 to 410.
414. The combination of any one of embodiments 322 to 410, wherein the first or second variant Fc region comprises the amino acid sequence of SEQ ID NO:252 with a substitution at 1, 2, 3, 4, 5 or 6 of positions 233, 234, 235, 236, 237, 239, 265, 266, 267, 268, 269, 297, 299, 322, 327, 328, 329, 330, 331 and 332, optionally wherein one or more of the substitutions are substitutions recited in any one of embodiments 324 to 410.
415. The combination of any one of embodiments 322 to 414, wherein the first or second variant Fc region comprises an amino acid sequence which is at least 90% identical to SEQ ID NO:253.
416. The combination of any one of embodiments 322 to 414, wherein the first or second variant Fc region comprises an amino acid sequence which is at least 95% identical to SEQ ID NO:253.
417. The combination of any one of embodiments 322 to 414, wherein the first or second variant Fc region comprises the amino acid sequence of SEQ ID NO:253 modified with the substitutions recited in any one of embodiments 324 to 410.
418. The combination of any one of embodiments 322 to 414, wherein the first or second variant Fc region comprises the amino acid sequence of SEQ ID NO:253 with a substitution at 1, 2, 3, 4, 5 or 6 of positions 233, 234, 235, 236, 237, 239, 265, 266, 267, 268, 269, 297, 299, 322, 327, 328, 329, 330, 331 and 332, optionally wherein one or more of the substitutions are substitutions recited in any one of embodiments 324 to 410.
419. The combination of any one of embodiments 322 to 414, wherein the first or second variant Fc region comprises an amino acid sequence which is at least 90% identical to SEQ ID NO:254.
420. The combination of any one of embodiments 322 to 414, wherein the first or second variant Fc region comprises an amino acid sequence which is at least 95% identical to SEQ ID NO:254.
421. The combination of any one of embodiments 322 to 414, wherein the first or second variant Fc region comprises the amino acid sequence of SEQ ID NO:254 modified with the substitutions recited in any one of embodiments 324 to 410.
422. The combination of any one of embodiments 322 to 414, wherein the first or second variant Fc region comprises the amino acid sequence of SEQ ID NO:254 with a substitution at 1, 2, 3, 4, 5 or 6 of positions 233, 234, 235, 236, 237, 239, 265, 266, 267, 268, 269, 297, 299, 322, 327, 328, 329, 330, 331 and 332, optionally wherein one or more of the substitutions are substitutions recited in any one of embodiments 324 to 410.
423. The combination of any one of embodiments 322 to 422, wherein the first or second variant Fc region comprises an amino acid sequence which is at least 90% identical to SEQ ID NO:251.
424. The combination of any one of embodiments 322 to 422, wherein the first or second variant Fc region comprises an amino acid sequence which is at least 95% identical to SEQ ID NO:251.
425. The combination of any one of embodiments 322 to 422, wherein the first or second variant Fc region comprises the amino acid sequence of SEQ ID NO:251 modified with the substitutions recited in any one of embodiments 324 to 410.
426. The combination of any one of embodiments 322 to 422, wherein the first or second variant Fc region comprises the amino acid sequence of SEQ ID NO:251 with a substitution at 1, 2, 3, 4, 5 or 6 of positions 233, 234, 235, 236, 237, 239, 265, 266, 267, 268, 269, 297, 299, 322, 327, 328, 329, 330, 331 and 332, optionally wherein one or more of the substitutions are substitutions recited in any one of embodiments 324 to 410.
427. The combination of any one of embodiments 40 to 269, wherein the CD19 binding molecule comprises an Fc domain.
428. The combination of embodiment 427, wherein the Fc domain is an Fc heterodimer.
429. The combination of embodiment 428, wherein the Fc heterodimer comprises any of the Fc modifications set forth in Table 4.
430. The combination of embodiment 428, wherein the Fc heterodimer comprises knob-in-hole (“KIH”) modifications.
431. The combination of any one of embodiments to 428 to 430, wherein the CD19 binding molecule comprises at least one of the Fc modifications designated as Fc 1 through Fc 150.
432. The combination of embodiment 431, wherein the CD19 binding molecule comprises at least one of the Fc modifications designated as Fc 1 through Fc 5.
433. The combination of embodiment 431, wherein the CD19 binding molecule comprises at least one of the Fc modifications designated as Fc 6 through Fc 10.
434. The combination of embodiment 431, wherein the CD19 binding molecule comprises at least one of the Fc modifications designated as Fc 11 through Fc 15.
435. The combination of embodiment 431, wherein the CD19 binding molecule comprises at least one of the Fc modifications designated as Fc 16 through Fc 20.
436. The combination of embodiment 431, wherein the CD19 binding molecule comprises at least one of the Fc modifications designated as Fc 21 through Fc 25.
437. The combination of embodiment 431, wherein the CD19 binding molecule comprises at least one of the Fc modifications designated as Fc 26 through Fc 30.
438. The combination of embodiment 431, wherein the CD19 binding molecule comprises at least one of the Fc modifications designated as Fc 31 through Fc 35.
439. The combination of embodiment 431, wherein the CD19 binding molecule comprises at least one of the Fc modifications designated as Fc 36 through Fc 40.
440. The combination of embodiment 431, wherein the CD19 binding molecule comprises at least one of the Fc modifications designated as Fc 41 through Fc 45.
441. The combination of embodiment 431, wherein the CD19 binding molecule comprises at least one of the Fc modifications designated as Fc 46 through Fc 50.
442. The combination of embodiment 431, wherein the CD19 binding molecule comprises at least one of the Fc modifications designated as Fc 51 through Fc 55.
443. The combination of embodiment 431, wherein the CD19 binding molecule comprises at least one of the Fc modifications designated as Fc 56 through Fc 60.
444. The combination of embodiment 431, wherein the CD19 binding molecule comprises at least one of the Fc modifications designated as Fc 61 through Fc 65.
445. The combination of embodiment 431, wherein the CD19 binding molecule comprises at least one of the Fc modifications designated as Fc 66 through Fc 70.
446. The combination of embodiment 431, wherein the CD19 binding molecule comprises at least one of the Fc modifications designated as Fc 71 through Fc 75.
447. The combination of embodiment 431, wherein the CD19 binding molecule comprises at least one of the Fc modifications designated as Fc 76 through Fc 80.
448. The combination of embodiment 431, wherein the CD19 binding molecule comprises at least one of the Fc modifications designated as Fc 81 through Fc 85.
449. The combination of embodiment 431, wherein the CD19 binding molecule comprises at least one of the Fc modifications designated as Fc 86 through Fc 90.
450. The combination of embodiment 431, wherein the CD19 binding molecule comprises at least one of the Fc modifications designated as Fc 91 through Fc 95.
451. The combination of embodiment 431, wherein the CD19 binding molecule comprises at least one of the Fc modifications designated as Fc 96 through Fc 100.
452. The combination of embodiment 431, wherein the CD19 binding molecule comprises at least one of the Fc modifications designated as Fc 101 through Fc 105.
453. The combination of embodiment 431, wherein the CD19 binding molecule comprises at least one of the Fc modifications designated as Fc 106 through Fc 110.
454. The combination of embodiment 431, wherein the CD19 binding molecule comprises at least one of the Fc modifications designated as Fc 111 through Fc 115.
455. The combination of embodiment 431, wherein the CD19 binding molecule comprises at least one of the Fc modifications designated as Fc 116 through Fc 120.
456. The combination of embodiment 431, wherein the CD19 binding molecule comprises at least one of the Fc modifications designated as Fc 121 through Fc 125.
457. The combination of embodiment 431, wherein the CD19 binding molecule comprises at least one of the Fc modifications designated as Fc 126 through Fc 130.
458. The combination of embodiment 431, wherein the CD19 binding molecule comprises at least one of the Fc modifications designated as Fc 131 through Fc 135.
459. The combination of embodiment 431, wherein the CD19 binding molecule comprises at least one of the Fc modifications designated as Fc 136 through Fc 140.
460. The combination of embodiment 431, wherein the CD19 binding molecule comprises at least one of the Fc modifications designated as Fc 141 through Fc 145.
461. The combination of embodiment 431, wherein the CD19 binding molecule comprises at least one of the Fc modifications designated as Fc 146 through Fc 150.
462. The combination of any one of embodiments 427 to 461, wherein the Fc domain of the CD19 binding molecule has altered effector function.
463. The combination of embodiment 462, wherein the Fc domain has altered binding to one or more Fc receptors.
464. The combination of embodiment 463, wherein the one or more Fc receptors comprise FcRN.
465. The combination of embodiment 463 or embodiment 464, wherein the one or more Fc receptors comprise leukocyte receptors.
466. The combination of any one of embodiments 427 to 465, wherein the Fc has modified disulfide bond architecture.
467. The combination of any one of embodiments 427 to 466, wherein the Fc has altered glycosylation patterns.
468. The combination of any one of embodiments 427 to 467, wherein the Fc comprises a hinge region.
469. The combination of embodiment 468, wherein the hinge region comprises any one of the hinge regions described in Section 7.2.2.2
470. The combination of embodiment 469, wherein the hinge region comprises the amino acid sequence of the hinge region designated H1.
471. The combination of embodiment 469, wherein the hinge region comprises the amino acid sequence of the hinge region designated H2.
472. The combination of embodiment 469, wherein the hinge region comprises the amino acid sequence of the hinge region designated H3.
473. The combination of embodiment 469, wherein the hinge region comprises the amino acid sequence of the hinge region designated H4.
474. The combination of embodiment 469, wherein the hinge region comprises the amino acid sequence of the hinge region designated H5.
475. The combination of embodiment 469, wherein the hinge region comprises the amino acid sequence of the hinge region designated H6.
476. The combination of embodiment 469, wherein the hinge region comprises the amino acid sequence of the hinge region designated H7.
477. The combination of embodiment 469, wherein the hinge region comprises the amino acid sequence of the hinge region designated H8.
478. The combination of embodiment 469, wherein the hinge region comprises the amino acid sequence of the hinge region designated H9.
479. The combination of embodiment 469, wherein the hinge region comprises the amino acid sequence of the hinge region designated H10.
480. The combination of embodiment 469, wherein the hinge region comprises the amino acid sequence of the hinge region designated H11.
481. The combination of embodiment 469, wherein the hinge region comprises the amino acid sequence of the hinge region designated H12.
482. The combination of embodiment 469, wherein the hinge region comprises the amino acid sequence of the hinge region designated H13.
483. The combination of embodiment 469, wherein the hinge region comprises the amino acid sequence of the hinge region designated H14.
484. The combination of embodiment 469, wherein the hinge region comprises the amino acid sequence of the hinge region designated H15.
485. The combination of embodiment 469, wherein the hinge region comprises the amino acid sequence of the hinge region designated H16.
486. The combination of embodiment 469, wherein the hinge region comprises the amino acid sequence of the hinge region designated H17.
487. The combination of embodiment 469, wherein the hinge region comprises the amino acid sequence of the hinge region designated H18.
488. The combination of embodiment 469, wherein the hinge region comprises the amino acid sequence of the hinge region designated H19.
489. The combination of embodiment 469, wherein the hinge region comprises the amino acid sequence of the hinge region designated H20.
490. The combination of embodiment 469, wherein the hinge region comprises the amino acid sequence of the hinge region designated H21.
491. The combination of any one of embodiments 40 to 490, wherein the CD19 binding molecule comprises at least one scFv domain.
492. The combination of embodiment 491, wherein at least one scFv comprises a linker connecting the VH and VL domains.
493. The combination of embodiment 492, wherein the linker is 5 to 25 amino acids in length.
494. The combination of embodiment 493, wherein the linker is 12 to 20 amino acids in length.
495. The combination of any one of embodiments 492 to 494, wherein the linker is a charged linker and/or a flexible linker.
496. The combination of any one of embodiments 492 to 495, wherein the linker is selected from any one of linkers L1 through L54.
497. The combination of embodiment 496, wherein the linker region comprises the amino acid sequence of the linker designated L1.
498. The combination of embodiment 496, wherein the linker region comprises the amino acid sequence of the linker designated L2.
499. The combination of embodiment 496, wherein the linker region comprises the amino acid sequence of the linker designated L3.
500. The combination of embodiment 496, wherein the linker region comprises the amino acid sequence of the linker designated L4.
501. The combination of embodiment 496, wherein the linker region comprises the amino acid sequence of the linker designated L5.
502. The combination of embodiment 496, wherein the linker region comprises the amino acid sequence of the linker designated L6.
503. The combination of embodiment 496, wherein the linker region comprises the amino acid sequence of the linker designated L7.
504. The combination of embodiment 496, wherein the linker region comprises the amino acid sequence of the linker designated L8.
505. The combination of embodiment 496, wherein the linker region comprises the amino acid sequence of the linker designated L9.
506. The combination of embodiment 496, wherein the linker region comprises the amino acid sequence of the linker designated L10.
507. The combination of embodiment 496, wherein the linker region comprises the amino acid sequence of the linker designated L11.
508. The combination of embodiment 496, wherein the linker region comprises the amino acid sequence of the linker designated L12.
509. The combination of embodiment 496, wherein the linker region comprises the amino acid sequence of the linker designated L13.
510. The combination of embodiment 496, wherein the linker region comprises the amino acid sequence of the linker designated L14.
511. The combination of embodiment 496, wherein the linker region comprises the amino acid sequence of the linker designated L15.
512. The combination of embodiment 496, wherein the linker region comprises the amino acid sequence of the linker designated L16.
513. The combination of embodiment 496, wherein the linker region comprises the amino acid sequence of the linker designated L17.
514. The combination of embodiment 496, wherein the linker region comprises the amino acid sequence of the linker designated L18.
515. The combination of embodiment 496, wherein the linker region comprises the amino acid sequence of the linker designated L19.
516. The combination of embodiment 496, wherein the linker region comprises the amino acid sequence of the linker designated L20.
517. The combination of embodiment 496, wherein the linker region comprises the amino acid sequence of the linker designated L21.
518. The combination of embodiment 496, wherein the linker region comprises the amino acid sequence of the linker designated L22.
519. The combination of embodiment 496, wherein the linker region comprises the amino acid sequence of the linker designated L23.
520. The combination of embodiment 496, wherein the linker region comprises the amino acid sequence of the linker designated L24.
521. The combination of embodiment 496, wherein the linker region comprises the amino acid sequence of the linker designated L25.
522. The combination of embodiment 496, wherein the linker region comprises the amino acid sequence of the linker designated L26.
523. The combination of embodiment 496, wherein the linker region comprises the amino acid sequence of the linker designated L27.
524. The combination of embodiment 496, wherein the linker region comprises the amino acid sequence of the linker designated L28.
525. The combination of embodiment 496, wherein the linker region comprises the amino acid sequence of the linker designated L29.
526. The combination of embodiment 496, wherein the linker region comprises the amino acid sequence of the linker designated L30.
527. The combination of embodiment 496, wherein the linker region comprises the amino acid sequence of the linker designated L31.
528. The combination of embodiment 496, wherein the linker region comprises the amino acid sequence of the linker designated L32.
529. The combination of embodiment 496, wherein the linker region comprises the amino acid sequence of the linker designated L33.
530. The combination of embodiment 496, wherein the linker region comprises the amino acid sequence of the linker designated L34.
531. The combination of embodiment 496, wherein the linker region comprises the amino acid sequence of the linker designated L35.
532. The combination of embodiment 496, wherein the linker region comprises the amino acid sequence of the linker designated L36.
533. The combination of embodiment 496, wherein the linker region comprises the amino acid sequence of the linker designated L37.
534. The combination of embodiment 496, wherein the linker region comprises the amino acid sequence of the linker designated L38.
535. The combination of embodiment 496, wherein the linker region comprises the amino acid sequence of the linker designated L39.
536. The combination of embodiment 496, wherein the linker region comprises the amino acid sequence of the linker designated L40.
537. The combination of embodiment 496, wherein the linker region comprises the amino acid sequence of the linker designated L41.
538. The combination of embodiment 496, wherein the linker region comprises the amino acid sequence of the linker designated L42.
539. The combination of embodiment 496, wherein the linker region comprises the amino acid sequence of the linker designated L43.
540. The combination of embodiment 496, wherein the linker region comprises the amino acid sequence of the linker designated L44.
541. The combination of embodiment 496, wherein the linker region comprises the amino acid sequence of the linker designated L45.
542. The combination of embodiment 496, wherein the linker region comprises the amino acid sequence of the linker designated L46.
543. The combination of embodiment 496, wherein the linker region comprises the amino acid sequence of the linker designated L47.
544. The combination of embodiment 496, wherein the linker region comprises the amino acid sequence of the linker designated L48.
545. The combination of embodiment 496, wherein the linker region comprises the amino acid sequence of the linker designated L49.
546. The combination of embodiment 496, wherein the linker region comprises the amino acid sequence of the linker designated L50.
547. The combination of embodiment 496, wherein the linker region comprises the amino acid sequence of the linker designated L51.
548. The combination of embodiment 496, wherein the linker region comprises the amino acid sequence of the linker designated L52.
549. The combination of embodiment 496, wherein the linker region comprises the amino acid sequence of the linker designated L53.
550. The combination of embodiment 496, wherein the linker region comprises the amino acid sequence of the linker designated L54.
551. The combination of any one of embodiments 40 to 550, wherein the CD19 binding molecule comprises at least one Fab domain.
552. The combination of embodiment 551, wherein at least one Fab domain comprises any of the Fab heterodimerization modifications set forth in Table 2.
553. The combination of embodiment 552, wherein at least one Fab domain comprises the Fab heterodimerization modifications designated as F1.
554. The combination of embodiment 552, wherein at least one Fab domain comprises the Fab heterodimerization modifications designated as F2.
555. The combination of embodiment 552, wherein at least one Fab domain comprises the Fab heterodimerization modifications designated as F3.
556. The combination of embodiment 552, wherein at least one Fab domain comprises the Fab heterodimerization modifications designated as F4.
557. The combination of embodiment 552, wherein at least one Fab domain comprises the Fab heterodimerization modifications designated as F5.
558. The combination of embodiment 552, wherein at least one Fab domain comprises the Fab heterodimerization modifications designated as F6.
559. The combination of embodiment 552, wherein at least one Fab domain comprises the Fab heterodimerization modifications designated as F7.
560. The combination of any one of embodiments 40 to 559, wherein the CD19 binding molecule comprises at least two ABMs, an ABM and an ABM chain, or two ABM chains connected to one another via a linker.
561. The combination of embodiment 560, wherein the linker is 5 to 25 amino acids in length.
562. The combination of embodiment 561, wherein the linker is 12 to 20 amino acids in length.
563. The combination of any one of embodiments 560 to 562, wherein the linker is a charged linker and/or a flexible linker.
564. The combination of any one of embodiments 560 to 563, wherein the linker is selected from any one of linkers L1 through L54.
565. The combination of embodiment 2, wherein the CD19 binding molecule is trispecific binding molecule (TBM) comprising:
   (a) an antigen-binding module 1 (ABM1) that binds specifically to CD19 and comprises CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19;
   (b) an antigen-binding module 2 (ABM2) that binds specifically to a component of a human T-cell receptor (TCR) complex; and
   (c) an antigen-binding module 3 (ABM3) that binds specifically to human CD2.
566. The combination of embodiment 565, wherein the CD19 binding molecule is trivalent.
567. The combination of embodiment 565 or 566, in which ABM1 is a Fab.
568. The combination of any one of embodiments 565 to 567, wherein ABM1 comprises a VH having the amino acid sequence of SEQ ID NO:13 and a VL having the amino acid sequence of SEQ ID NO:26.
569. The combination of any one of embodiments 565 to 569, wherein the component of the TCR complex is CD3.
570. The combination of embodiment 569, wherein ABM2 is an anti-CD3 antibody or an antigen-binding domain thereof.
571. The combination of embodiment 570, wherein ABM2 comprises the CDR sequences of CD3hi.
572. The combination of embodiment 570 or 571, wherein ABM2 comprises the heavy and light chain variable sequences of CD3hi, as set forth in Table 9A.
573. The combination of any one of embodiments 570 to 572, wherein the anti-CD3 antibody or antigen-binding domain thereof is in the form of a scFv.
574. The combination of embodiment 573, wherein ABM2 comprises the amino acid sequence of the scFv designated as CD3hi in Table 9A.
575. The combination of any one of embodiments 565 to 574, wherein ABM3 is a CD58 moiety.
576. The combination of any one of embodiments 565 to 575, wherein ABM3 comprises the amino acid sequence of CD58-6 as set forth in Table 12.
577. The combination of any one of embodiments 565 to 576, which comprises an Fc domain.
578. The combination of any one of embodiments 565 to 576, which comprises a first variant Fc region and a second variant Fc region that together form an Fc heterodimer.
579. The combination of embodiment 2, wherein the CD19 binding molecule is a trispecific binding molecule (TBM) comprising:
   (a) an antigen-binding module 1 (ABM1) that binds specifically to CD19 and which is a Fab comprising: (i) CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19; or (ii) CDR-H1, CDR-H2, and CDR-H3 having the amino acid sequences of SEQ ID NO:30, SEQ ID NO:31, and SEQ ID NO:32, and CDR-L1, CDR-L2, and CDR-L3 having the amino acid sequences of SEQ ID NO:43, SEQ ID NO:44, and SEQ ID NO:45;
   (b) an antigen-binding module 2 (ABM2) that binds specifically to CD3 and which comprises the amino acid sequence of the scFv designated as CD3hi in Table 9A;
   (c) an antigen-binding module 3 (ABM3) that binds specifically to human CD2 and which comprises the amino acid sequence of CD58-6 as set forth in Table 12; and
   (d) an Fc domain.
580. The combination of embodiment 2, wherein the CD19 binding molecule is a trispecific binding molecule (TBM) comprising:
   (a) an antigen-binding module 1 (ABM1) that binds specifically to CD19 and which is a Fab comprising: (i) CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19; or (ii) CDR-H1, CDR-H2, and CDR-H3 having the amino acid sequences of SEQ ID NO:30, SEQ ID NO:31, and SEQ ID NO:32, and CDR-L1, CDR-L2, and CDR-L3 having the amino acid sequences of SEQ ID NO:43, SEQ ID NO:44, and SEQ ID NO:45;
   (b) an antigen-binding module 2 (ABM2) that binds specifically to CD3 and which comprises the amino acid sequence of the scFv designated as CD3hi in Table 9A;
   (c) an antigen-binding module 3 (ABM3) that binds specifically to human CD2 and which comprises the amino acid sequence of CD58-6 as set forth in Table 12; and
   (d) an Fc domain.
581. The combination of embodiment 579 or embodiment 580, wherein ABM1 comprises CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19.
582. The combination of any one of embodiments 579 to 581, wherein ABM1 comprises a VH having the amino acid sequence of SEQ ID NO:13 and a VL having the amino acid sequence of SEQ ID NO:26.
583. The combination of any one of embodiments 565 to 582, wherein the CD19 binding molecule has the configuration depicted in FIG. 21 and referred to as T2 in Section 7.2.4.1.
584. The combination of any one of embodiments 565 to 583, wherein the CD19 binding molecule comprises an Fc domain which is an Fc heterodimer.
585. The combination of embodiment 584, wherein the Fc heterodimer of the CD19 binding molecule comprises knob-in-hole (“KIH”) modifications.
586. The combination of embodiment 585, wherein the CD19 binding molecule comprises at least one of the Fc modifications designated as Fc 121 through Fc 125.
587. The combination of any one of embodiments 584 to 586, wherein the Fc domain of the CD19 binding molecule has altered effector function.
588. The combination of embodiment 587, wherein the Fc domain of the CD19 binding molecule has altered binding to one or more Fc receptors.
589. The combination of any one of embodiments 584 to 588, wherein the Fc domain of the CD19 binding molecule is a silent IgG1 comprising the D265A mutation.
590. The combination of any one of embodiments 584 to 589, wherein the Fc domain of the CD19 binding molecule is a silent IgG1 comprising the D265A and P329A mutations.
591. The combination of any one of embodiments 565 to 590, wherein: the antigen-binding module 1 (ABM1) that binds specifically to CD19 comprises a VH is fused to a constant human IgG1 domain CH1 and a VL fused to a constant human kappa sequence.
592. The combination of any one of embodiments 584 to 591, wherein the Fc domain of the CD19 binding molecule is a human IgG1 Fc domain which comprises:
   (a) a first CH3 domain comprising the modification T366W; and
   (b) a second CH3 domain that heterodimerizes with the first CH3 domain and comprises the modifications T366S, L368A and Y407V.
593. The combination of any one of embodiments 584 to 592, wherein the Fc domain of the CD19 binding molecule is a human IgG1 Fc domain which comprises:
   (a) a first CH3 domain comprising the modification S354C and
   (b) a second CH3 domain comprises the modification Y349C.
594. The combination of any one of embodiments 584 to 593, wherein the Fc domain of the CD19 binding molecule comprises a human IgG1 sequence of SEQ ID NO:251 with a mutation at 1, 2, 3, 4, 5 or 6 positions of positions 233, 234, 235, 236, 237, 239, 265, 266, 267, 268, 269, 297, 299, 322, 327, 328, 329, 330, 331 and 332 (EU numbering).
595. The combination of any one of embodiments 584 to 594, wherein the Fc domain of the CD19 binding molecule comprises a human IgG1 Fc domain modified by substituting the aspartate residue at position 265 with an alanine residue, the asparagine residue at position 297 with an alanine residue and the proline residue at position 329 with an alanine residue (D265A/N297A/P329A).
596. The combination of any one of embodiments 582 to 595, wherein in the CD19 binding molecule a) the VH is fused to a constant human IgG1 CH1 domain and is joined by a linker to b) an antigen-binding module 2 (ABM2) that binds specifically to CD3 which comprises an scFv.
597. The combination of any of embodiments 582 to 595, wherein a) the VH is fused to a constant human IgG1 CH1 domain and is joined by a linker to b) an antigen-binding module 2 (ABM2) that binds specifically to CD3 which comprises an scFv which is joined by a linker to the d) Fc domain.
598. The combination of any of embodiments 565 to 597, wherein the CD19 binding molecule comprises a first half antibody comprising a) an antigen-binding module 1 (ABM1) that binds specifically to CD19; b) an antigen-binding module 2 (ABM2) that binds specifically to CD3 and which comprises an scFv and d) an Fc region; and a second half antibody comprising an antigen-binding module 3 (ABM3) that binds specifically to human CD2 and which comprises a CD58 IgV domain and d) an Fc region, wherein the Fc region in the first half antibody and the Fc region in the second half antibody form a Fc heterodimer.
599. The combination of embodiment 598, wherein the CD19 binding molecule comprises a first half antibody which comprises a heavy chain comprising the amino acid sequence of SEQ ID NO:63; and a light chain comprising the amino acid sequence of SEQ ID NO:64; and a second half antibody comprising the amino acid sequence of SEQ ID NO:65.
600. The combination of embodiment 598, wherein the CD19 binding molecule comprises a first half antibody which comprises a heavy chain comprising the amino acid sequence of SEQ ID NO:74; and a light chain comprising the amino acid sequence of SEQ ID NO:64; and a second half antibody comprising the amino acid sequence of SEQ ID NO:75 or SEQ ID NO:86.
601. The combination of embodiment 2, wherein the CD19 binding molecule comprises:
   (a) first half antibody heavy chain whose amino acid sequence comprises the amino acid sequence of SEQ ID NO:63 and a Fc sequence;
   (b) a first half antibody light chain whose amino acid sequence comprises the amino acid sequence of SEQ ID NO:64;
   (c) a second half antibody whose amino acid sequence comprises the amino acid sequence of SEQ ID NO:65 and a Fc sequence.
602. The combination of embodiment 2, wherein the CD19 binding molecule comprises:
   (a) a first polypeptide whose amino acid sequence comprises the amino acid sequence of SEQ ID NO:74;
   (b) a second polypeptide whose amino acid sequence comprises the amino acid sequence of SEQ ID NO:64; and
   (c) a third polypeptide whose amino acid sequence comprises the amino acid sequence of SEQ ID NO:75 or SEQ ID NO:86.
603. The combination of embodiment 2, wherein the CD19 binding molecule comprises:
   (a) a first polypeptide whose amino acid sequence comprises the amino acid sequence of SEQ ID NO:76;
   (b) a second polypeptide whose amino acid sequence comprises the amino acid sequence of SEQ ID NO:64; and
   (c) a third polypeptide whose amino acid sequence comprises the amino acid sequence of SEQ ID NO:75 or SEQ ID NO:86.
604. The combination of embodiment 2, wherein the CD19 binding molecule comprises:
   (a) a first polypeptide whose amino acid sequence comprises the amino acid sequence of SEQ ID NO:74;
   (b) a second polypeptide whose amino acid sequence comprises the amino acid sequence of SEQ ID NO:64; and
   (c) a third polypeptide whose amino acid sequence comprises the amino acid sequence of SEQ ID NO:86.
605. The combination of embodiment 2, wherein the CD19 binding molecule comprises:
   (a) a first polypeptide whose amino acid sequence comprises the amino acid sequence of SEQ ID NO:1120;
   (b) a second polypeptide whose amino acid sequence comprises the amino acid sequence of SEQ ID NO: 1122; and
   (c) a third polypeptide whose amino acid sequence comprises the amino acid sequence of SEQ ID NO:1110.
606. The combination of embodiment 2, wherein the CD19 binding molecule comprises:
   (a) a first polypeptide whose amino acid sequence comprises the amino acid sequence of SEQ ID NO:1127;
   (b) a second polypeptide whose amino acid sequence comprises the amino acid sequence of SEQ ID NO: 1129; and
   (c) a third polypeptide whose amino acid sequence comprises the amino acid sequence of SEQ ID NO:1110.
607. The combination of embodiment 2, wherein the CD19 binding molecule comprises:
   (a) a first polypeptide whose amino acid sequence comprises the amino acid sequence of SEQ ID NO:1124;
   (b) a second polypeptide whose amino acid sequence comprises the amino acid sequence of SEQ ID NO: 1126; and
   (c) a third polypeptide whose amino acid sequence comprises the amino acid sequence of SEQ ID NO:1110.
608. The combination of embodiment 2, wherein the CD19 binding molecule comprises:
   (a) a first polypeptide whose amino acid sequence comprises the amino acid sequence of SEQ ID NO:1134;
   (b) a second polypeptide whose amino acid sequence comprises the amino acid sequence of SEQ ID NO: 1135; and
   (c) a third polypeptide whose amino acid sequence comprises the amino acid sequence of SEQ ID NO:1110.
609. The combination of embodiment 2, wherein the CD19 binding molecule comprises:
   (a) a first polypeptide whose amino acid sequence comprises the amino acid sequence of SEQ ID NO:1136;
   (b) a second polypeptide whose amino acid sequence comprises the amino acid sequence of SEQ ID NO: 1137; and
   (c) a third polypeptide whose amino acid sequence comprises the amino acid sequence of SEQ ID NO:1110.
610. The combination of embodiment 2, wherein the CD19 binding molecule comprises:
   (a) a first polypeptide whose amino acid sequence comprises the amino acid sequence of SEQ ID NO:1138;
   (b) a second polypeptide whose amino acid sequence comprises the amino acid sequence of SEQ ID NO: 1139; and
   (c) a third polypeptide whose amino acid sequence comprises the amino acid sequence of SEQ ID NO:1110.
611. The combination of embodiment 2, wherein the CD19 binding molecule comprises:
   (a) a first polypeptide whose amino acid sequence comprises the amino acid sequence of SEQ ID NO:1140;
   (b) a second polypeptide whose amino acid sequence comprises the amino acid sequence of SEQ ID NO:1141; and
   (c) a third polypeptide whose amino acid sequence comprises the amino acid sequence of SEQ ID NO:1110.
612. The combination of embodiment 2, wherein the CD19 binding molecule comprises:
   (a) a first polypeptide whose amino acid sequence comprises the amino acid sequence of SEQ ID NO:1131;
   (b) a second polypeptide whose amino acid sequence comprises the amino acid sequence of SEQ ID NO: 1132; and
   (c) a third polypeptide whose amino acid sequence comprises the amino acid sequence of SEQ ID NO: 1133.
613. The combination of embodiment 2, wherein the CD19 binding molecule comprises the sequences of a construct shown in Table 20A-1, 20A-2, 20B, or 20C, preferably Table 20C.
614. The combination of embodiment 2, wherein the CD19 binding molecule is blinatumomab.
615. The combination of embodiment 2, wherein the CD19 binding molecule is coltuximab ravtansine.
616. The combination of embodiment 2, wherein the CD19 binding molecule is MOR208.
617. The combination of embodiment 2, wherein the CD19 binding molecule is MEDI-551.
618. The combination of embodiment 2, wherein the CD19 binding molecule is denintuzumab mafodotin.
619. The combination of embodiment 2, wherein the CD19 binding molecule is DI-B4.
620. The combination of embodiment 2, wherein the CD19 binding molecule is taplitumomabpaptox.
621. The combination of embodiment 2, wherein the CD19 binding molecule is XmAb 5871.
622. The combination of embodiment 2, wherein the CD19 binding molecule is MDX-1342.
623. The combination of embodiment 2, wherein the CD19 binding molecule is AFM11.
624. The combination of embodiment 2, wherein the CD19 binding molecule is MDX-1342.
625. The combination of embodiment 2, wherein the CD19 binding molecule is loncastuximab tesirine.
626. The combination of embodiment 2, wherein the CD19 binding molecule is GBR401.
627. The combination of embodiment 1, wherein the anti-CD19 agent is a population of cells that expresses a chimeric antigen receptor (“CAR”) molecule that binds CD19 (“a CAR composition”).
628. The combination of embodiment 627, wherein the CAR molecule comprises an anti-CD19 binding domain, a transmembrane domain, and an intracellular signaling domain.
629. The combination of embodiment 628, wherein the intracellular signaling domain comprises a costimulatory domain and a primary signaling domain.
630. The combination of embodiment 628 or embodiment 629, wherein the CAR molecule comprises an anti-CD19 binding domain comprising a light chain complementary determining region 1 (CDR-11), a light chain complementary determining region 2 (CDR-L2), a light chain complementary determining region 3 (CDR-L3), a heavy chain complementary determining region 1 (CDR-H1), a heavy chain complementary determining region 2 (CDR-H2), and a heavy chain complementary determining region 3 (CDR-H3) of an anti-CD19 binding domain.
631. The combination of any one of embodiments 628 to 630, wherein the CAR molecule comprises an scFv.
632. The combination of embodiment 631, wherein the anti-CD19 binding domain is a scFv comprising a heavy chain variable region (VH) attached to a heavy chain light region (VL) via a linker.
633. The combination of embodiment 632, wherein the linker comprises the amino acid sequence of SEQ ID NO:144.
634. The combination of any one of embodiments 628 to 633, wherein the CAR molecule is a murine CAR molecule.
635. The combination of any one of embodiments 628 to 634, wherein the CAR molecule comprises one or more of CDR-H1, CDR-H2 and CDR-H3 of any CD19 scFv domain amino acid sequence listed in Table 17 and one or more of CDR-L1, CDR-L2 and CDR-L3 of any CD19 scFv domain amino acid sequence listed in Table 17.
636. The combination of any one of embodiments 628 to 635, wherein the CAR molecule comprises a VH of any CD19 scFv domain amino acid sequence listed in Table 17 and a VL of any CD19 scFv domain amino acid sequence listed in Table 17.
637. The combination of any one of embodiments 628 to 636, wherein the CAR molecule comprises a CD19 scFv domain having an amino acid that is at least 95% identical to a CD19 scFv amino acid sequence listed in Table 17.
638. The combination of embodiment 637, wherein the CD19 scFv domain comprises the amino acid sequence of SEQ ID NO:149 or an amino acid sequence having at least 95% sequence identity to the amino acid sequence of SEQ ID NO:149.
639. The combination of embodiment 637, wherein the CD19 scFv domain comprises the amino acid sequence of SEQ ID NO:194 or an amino acid sequence having at least 95% sequence identity to the amino acid sequence of SEQ ID NO:194.
640. The combination of embodiment 637, wherein the CD19 scFv domain comprises the amino acid sequence of SEQ ID NO:196 or an amino acid sequence having at least 95% sequence identity to the amino acid sequence of SEQ ID NO:196.
641. The combination of embodiment 637, wherein the CD19 scFv domain comprises the amino acid sequence of SEQ ID NO:199 or an amino acid sequence having at least 95% sequence identity to the amino acid sequence of SEQ ID NO:199.
642. The combination of any one of embodiments 628 to 636, wherein the CAR molecule comprises a full-length CD19 CAR amino acid sequence listed in Table 17 or an amino acid sequence having at least 95% sequence identity to a full-length CD19 CAR amino acid sequence listed in Table 17.
643. The combination of embodiment 642, wherein the CAR molecule comprises the amino acid sequence of SEQ ID NO:195 or an amino acid sequence having at least 95% sequence identity to the amino acid sequence of SEQ ID NO:195.
644. The combination of embodiment 642, wherein the CAR molecule comprises the amino acid sequence of SEQ ID NO:197 or an amino acid sequence having at least 95% sequence identity to the amino acid sequence of SEQ ID NO:197.
645. The combination of embodiment 642, wherein the CAR molecule comprises the amino acid sequence of SEQ ID NO:198 or an amino acid sequence having at least 95% sequence identity to the amino acid sequence of SEQ ID NO:198.
646. The combination of embodiment 642, wherein the CAR molecule comprises the amino acid sequence of SEQ ID NO:200 or an amino acid sequence having at least 95% sequence identity to the amino acid sequence of SEQ ID NO:200.
647. The combination of embodiment 642, wherein the CAR molecule comprises the amino acid sequence of residues 22-486 of SEQ ID NO:148 or an amino acid sequence having at least 95% sequence identity to the amino acid sequence of residues 22-486 of SEQ ID NO:148.
648. The combination of any one of embodiments 628 to 633, wherein the CAR molecule is a humanized CAR molecule.
649. The combination of any one of embodiments 628 to 633 and 648, wherein the CAR molecule comprises one or more of CDR-H1, CDR-H2 and CDR-H3 of any CD19 scFv domain amino acid sequence listed in Table 16 and one or more of CDR-L1, CDR-L2 and CDR-L3 of any CD19 scFv domain amino acid sequence listed in Table 16.
650. The combination of any one of embodiments 628 to 633, 648 and 649, wherein the CAR molecule comprises a VH of any CD19 scFv domain amino acid sequence listed in Table 16 and a VL of any CD19 scFv domain amino acid sequence listed in Table 16.
651. The combination of any one of embodiments 628 to 633 and 648 to 650, wherein the CAR molecule comprises a CD19 scFv domain amino acid sequence listed in Table 16 or an amino acid sequence having at least 95% sequence identity to a CD19 scFv domain amino acid sequence listed in Table 16.
652. The combination of embodiment 651, wherein the CD19 scFv domain comprises the amino acid sequence of SEQ ID NO:96 or an amino acid sequence having at least 95% sequence identity to the amino acid sequence of SEQ ID NO:96.
653. The combination of embodiment 651, wherein the CD19 scFv domain comprises the amino acid sequence of SEQ ID NO:97 or an amino acid sequence having at least 95% sequence identity to the amino acid sequence of SEQ ID NO:97.
654. The combination of embodiment 651, wherein the CD19 scFv domain comprises the amino acid sequence of SEQ ID NO:98 or an amino acid sequence having at least 95% sequence identity to the amino acid sequence of SEQ ID NO:98.
655. The combination of embodiment 651, wherein the CD19 scFv domain comprises the amino acid sequence of SEQ ID NO:99 or an amino acid sequence having at least 95% sequence identity to the amino acid sequence of SEQ ID NO:99.
656. The combination of embodiment 651, wherein the CD19 scFv domain comprises the amino acid sequence of SEQ ID NO:100 or an amino acid sequence having at least 95% sequence identity to the amino acid sequence of SEQ ID NO:100.
657. The combination of embodiment 651, wherein the CD19 scFv domain comprises the amino acid sequence of SEQ ID NO:101 or an amino acid sequence having at least 95% sequence identity to the amino acid sequence of SEQ ID NO:101.
658. The combination of embodiment 651, wherein the CD19 scFv domain comprises the amino acid sequence of SEQ ID NO:102 or an amino acid sequence having at least 95% sequence identity to the amino acid sequence of SEQ ID NO:102.
659. The combination of embodiment 651, wherein the CD19 scFv domain comprises the amino acid sequence of SEQ ID NO:103 or an amino acid sequence having at least 95% sequence identity to the amino acid sequence of SEQ ID NO:103.
660. The combination of embodiment 651, wherein the CD19 scFv domain comprises the amino acid sequence of SEQ ID NO:104 or an amino acid sequence having at least 95% sequence identity to the amino acid sequence of SEQ ID NO:104.
661. The combination of embodiment 651, wherein the CD19 scFv domain comprises the amino acid sequence of SEQ ID NO:105 or an amino acid sequence having at least 95% sequence identity to the amino acid sequence of SEQ ID NO:105.
662. The combination of embodiment 651, wherein the CD19 scFv domain comprises the amino acid sequence of SEQ ID NO:106 or an amino acid sequence having at least 95% sequence identity to the amino acid sequence of SEQ ID NO:106.
663. The combination of embodiment 651, wherein the CD19 scFv domain comprises the amino acid sequence of SEQ ID NO:107 or an amino acid sequence having at least 95% sequence identity to the amino acid sequence of SEQ ID NO:107.
664. The combination of any one of embodiments 628 to 633 and 648 to 651, wherein the CAR molecule comprises a full-length CD19 CAR amino acid sequence listed in Table 16 or an amino acid sequence having at least 95% sequence identity to a CD19 scFv domain amino acid sequence listed in Table 16.
665. The combination of embodiment 664, wherein the CAR molecule comprises the amino acid sequence of residues 22-486 of any one of SEQ ID NOs: 122-125 or 133, residues 22-491 of any one of SEQ ID NOs: 126-132, or an amino acid having at least 95% sequence identity to any of the foregoing sequences.
666. The combination of embodiment 665, wherein the CAR molecule comprises the amino acid sequence of residues 22-486 of SEQ ID NO:122 or an amino acid sequence having at least 95% sequence identity to residues 22-486 of SEQ ID NO:122.
667. The combination of embodiment 665, wherein the CAR molecule comprises the amino acid sequence of residues 22-486 of SEQ ID NO:123 or an amino acid sequence having at least 95% sequence identity to residues 22-486 of SEQ ID NO:123.
668. The combination of embodiment 665, wherein the CAR molecule comprises the amino acid sequence of residues 22-486 of SEQ ID NO:124 or an amino acid sequence having at least 95% sequence identity to residues 22-486 of SEQ ID NO:124.
669. The combination of embodiment 665, wherein the CAR molecule comprises the amino acid sequence of residues 22-486 of SEQ ID NO:125 or an amino acid sequence having at least 95% sequence identity to residues 22-486 of SEQ ID NO:125.
670. The combination of embodiment 665, wherein the CAR molecule comprises the amino acid sequence of residues 22-486 of SEQ ID NO:133 or an amino acid sequence having at least 95% sequence identity to residues 22-486 of SEQ ID NO:133.
671. The combination of embodiment 665, wherein the CAR molecule comprises the amino acid sequence of residues 22-491 of SEQ ID NO:126 or an amino acid sequence having at least 95% sequence identity to residues 22-491 of SEQ ID NO:126.
672. The combination of embodiment 665, wherein the CAR molecule comprises the amino acid sequence of residues 22-491 of SEQ ID NO:127 or an amino acid sequence having at least 95% sequence identity to residues 22-491 of SEQ ID NO:127.
673. The combination of embodiment 665, wherein the CAR molecule comprises the amino acid sequence of residues 22-491 of SEQ ID NO:128 or an amino acid sequence having at least 95% sequence identity to residues 22-491 of SEQ ID NO:128.
674. The combination of embodiment 665, wherein the CAR molecule comprises the amino acid sequence of residues 22-491 of SEQ ID NO:129 or an amino acid sequence having at least 95% sequence identity to residues 22-491 of SEQ ID NO:129.
675. The combination of embodiment 665, wherein the CAR molecule comprises the amino acid sequence of residues 22-491 of SEQ ID NO:130 or an amino acid sequence having at least 95% sequence identity to residues 22-491 of SEQ ID NO:130.
676. The combination of embodiment 665, wherein the CAR molecule comprises the amino acid sequence of residues 22-491 of SEQ ID NO:131 or an amino acid sequence having at least 95% sequence identity to residues 22-491 of SEQ ID NO:131.
677. The combination of embodiment 665, wherein the CAR molecule comprises the amino acid sequence of residues 22-491 of SEQ ID NO:132 or an amino acid sequence having at least 95% sequence identity to residues 22-491 of SEQ ID NO:132.
678. The combination of any one of embodiments 628 to 633, wherein the anti-CD19 binding domain comprises CDR-H1, CDR-H2, and CDR-H3 having the amino acid sequences of SEQ ID NO:1, SEQ ID NO:2, and SEQ ID NO:3, and CDR-L1, CDR-L2, and CDR-L3 having the amino acid sequences of SEQ ID NO:14, SEQ ID NO:15, and SEQ ID NO:16.
679. The combination of any one of embodiments 628 to 633, wherein the anti-CD19 binding domain comprises CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19.
680. The combination of any one of embodiments 628 to 633, wherein the anti-CD19 binding domain comprises CDR-H1, CDR-H2, and CDR-H3 having the amino acid sequences of SEQ ID NO:7, SEQ ID NO:8, and SEQ ID NO:9, and CDR-L1, CDR-L2, and CDR-L3 having the amino acid sequences of SEQ ID NO:20, SEQ ID NO:21, and SEQ ID NO:22.
681. The combination of any one of embodiments 628 to 633, wherein the anti-CD19 binding domain comprises CDR-H1, CDR-H2, and CDR-H3 having the amino acid sequences of SEQ ID NO:10, SEQ ID NO:11, and SEQ ID NO:12, and CDR-L1, CDR-L2, and CDR-L3 having the amino acid sequences of SEQ ID NO:23, SEQ ID NO:24, and SEQ ID NO:25
682. The combination of any one of embodiments 678 to 681, wherein the anti-CD19 binding domain comprises a VH having the amino acid sequence of SEQ ID NO:13 and/or a VL having the amino acid sequence of SEQ ID NO:26.
683. The combination of any one of embodiments 628 to 633, wherein the anti-CD19 binding domain comprises CDR-H1, CDR-H2, and CDR-H3 having the amino acid sequences of SEQ ID NO:27, SEQ ID NO:28, and SEQ ID NO:29, and CDR-L1, CDR-L2, and CDR-L3 having the amino acid sequences of SEQ ID NO:40, SEQ ID NO:41, and SEQ ID NO:42.
684. The combination of any one of embodiments 628 to 633, wherein the anti-CD19 binding domain comprises CDR-H1, CDR-H2, and CDR-H3 having the amino acid sequences of SEQ ID NO:30, SEQ ID NO:31, and SEQ ID NO:32, and CDR-L1, CDR-L2, and CDR-L3 having the amino acid sequences of SEQ ID NO:43, SEQ ID NO:44, and SEQ ID NO:45.
685. The combination of any one of embodiments 628 to 633, wherein the anti-CD19 binding domain comprises CDR-H1, CDR-H2, and CDR-H3 having the amino acid sequences of SEQ ID NO:33, SEQ ID NO:34, and SEQ ID NO:35, and CDR-L1, CDR-L2, and CDR-L3 having the amino acid sequences of SEQ ID NO:46, SEQ ID NO:47, and SEQ ID NO:48.
686. The combination of any one of embodiments 628 to 633, wherein the anti-CD19 binding domain comprises CDR-H1, CDR-H2, and CDR-H3 having the amino acid sequences of SEQ ID NO:36, SEQ ID NO:37, and SEQ ID NO:38, and CDR-L1, CDR-L2, and CDR-L3 having the amino acid sequences of SEQ ID NO:49, SEQ ID NO:50, and SEQ ID NO:51.
687. The combination of any one of embodiments 683 to 686, wherein the anti-CD19 binding domain comprises a VH having the amino acid sequence of SEQ ID NO: 39 and/or a VL having the amino acid sequence of SEQ ID NO: 52.
688. The combination of any one of embodiments 627 to 687, wherein the CAR molecule comprises a transmembrane domain of a protein chosen from: the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154.
689. The combination of any one of embodiments 627 to 688, wherein the CAR molecule comprises a hinge region.
690. The combination of any one of embodiments 627 to 689, wherein the CAR molecule comprises a costimulatory domain that is a functional signaling domain.
691. The combination of embodiment 690, wherein the costimulatory domain has an amino acid sequence from OX40, CD2, CD27, CD28, CDS, ICAM-1, LFA-1 (CD11a/CD18), ICOS (CD278), or 4-1 BB (CD137).
692. The combination of any one of embodiments 627 to 690, wherein the CAR molecule comprises an intracellular signaling domain comprising a functional signaling domain of 4-1BB and/or a functional signaling domain of CD3-zeta.
693. The combination of any one of embodiments 627 to 692, wherein the CAR molecule comprises a leader sequence.
694. The combination of any one of embodiments 627 to 632, wherein the CAR molecule comprises the amino acid sequence of 1928z (SEQ ID NO:201) with or without its signal (leader) sequence.
695. The combination of any one of embodiments 627 to 632, wherein the CAR molecule comprises the amino acid sequence of the scFv portion of 1928z (SEQ ID NO:201).
696. The combination of any one of embodiments 627 to 632, wherein the CAR molecule comprises the amino acid sequences of the heavy and light chain CDRs of 1928z (SEQ ID NO:201).
697. The combination of embodiment 627, wherein the CAR composition is tisagenlecleucel.
698. The combination of embodiment 627, wherein the CAR composition is axicabtagene ciloleucel.
699. The combination of embodiment 627, wherein the CAR composition is lisocabtagene maraleucel.
700. The combination of embodiment 627, wherein the CAR composition is brexucabtagene autoleucel.
701. The combination of any one of embodiments 1 to 700, wherein the B cell targeting agent is a B cell depleting agent.
702. The combination of any one of embodiments 1 to 701, wherein the B cell targeting agent is a BAFF receptor (BAFFR) binding molecule.
703. The combination of embodiment 702, wherein the BAFFR binding molecule is an antibody or antigen-binding domain thereof.
704. The combination of embodiment 702 or 703, wherein the BAFFR binding molecule comprises CDR-H1, CDR-H2, CDR-H3 having the amino acid sequences of ianalumab set forth in Table 18, and CDR-L1, CDR-L2, and CDR-L3 having the amino acid sequences of ianalumab set forth in Table 18.
705. The combination of embodiment 704, wherein the BAFFR binding molecule comprises a heavy chain variable region (VH) and a light chain variable region (VL) having the VH and VL sequences of ianalumab set forth in Table 18.
706. The combination of embodiment 705, wherein the BAFFR binding molecule is ianalumab.
707. The combination of embodiment 702 or 703, wherein the BAFFR binding molecule comprises CDR-H1, CDR-H2, CDR-H3 having the amino acid sequences of BAFFR-1 set forth in Table 19, and CDR-L1, CDR-L2, and CDR-L3 having the amino acid sequences of BAFFR-1 set forth in Table 19.
708. The combination of embodiment 702 or 703, wherein the BAFFR binding molecule comprises CDR-H1, CDR-H2, CDR-H3 having the amino acid sequences of BAFFR-2 set forth in Table 19, and CDR-L1, CDR-L2, and CDR-L3 having the amino acid sequences of BAFFR-2 set forth in Table 19.
709. The combination of embodiment 702 or 703, wherein the BAFFR binding molecule comprises CDR-H1, CDR-H2, CDR-H3 having the amino acid sequences of BAFFR-3 set forth in Table 19, and CDR-L1, CDR-L2, and CDR-L3 having the amino acid sequences of BAFFR-3 set forth in Table 19.
710. The combination of embodiment 702 or 703, wherein the BAFFR binding molecule comprises CDR-H1, CDR-H2, CDR-H3 having the amino acid sequences of BAFFR-4 set forth in Table 19, and CDR-L1, CDR-L2, and CDR-L3 having the amino acid sequences of BAFFR-4 set forth in Table 19.
711. The combination of embodiment 702 or 703, wherein the BAFFR binding molecule comprises CDR-H1, CDR-H2, CDR-H3 having the amino acid sequences of BAFFR-5 set forth in Table 19, and CDR-L1, CDR-L2, and CDR-L3 having the amino acid sequences of BAFFR-5 set forth in Table 19.
712. The combination of embodiment 702 or 703, wherein the BAFFR binding molecule comprises CDR-H1, CDR-H2, CDR-H3 having the amino acid sequences of BAFFR-5 set forth in Table 19, and CDR-L1, CDR-L2, and CDR-L3 having the amino acid sequences of BAFFR-5 set forth in Table 19.
713. The combination of embodiment 702 or 703, wherein the BAFFR binding molecule comprises CDR-H1, CDR-H2, CDR-H3 having the amino acid sequences of BAFFR-5 set forth in Table 19, and CDR-L1, CDR-L2, and CDR-L3 having the amino acid sequences of BAFFR-5 set forth in Table 19.
714. The combination of embodiment 702 or 703, wherein the BAFFR binding molecule comprises a heavy chain variable region (VH) and a light chain variable region (VL) having the VH and VL sequences of BAFFR-1 set forth in Table 19.
715. The combination of embodiment 702 or 703, wherein the BAFFR binding molecule comprises a heavy chain variable region (VH) and a light chain variable region (VL) having the VH and VL sequences of BAFFR-2 set forth in Table 19.
716. The combination of embodiment 702 or 703, wherein the BAFFR binding molecule comprises a heavy chain variable region (VH) and a light chain variable region (VL) having the VH and VL sequences of BAFFR-3 set forth in Table 19.
717. The combination of embodiment 702 or 703, wherein the BAFFR binding molecule comprises a heavy chain variable region (VH) and a light chain variable region (VL) having the VH and VL sequences of BAFFR-4 set forth in Table 19.
718. The combination of embodiment 702 or 703, wherein the BAFFR binding molecule comprises a heavy chain variable region (VH) and a light chain variable region (VL) having the VH and VL sequences of BAFFR-5 set forth in Table 19.
719. The combination of embodiment 702 or 703, wherein the BAFFR binding molecule comprises a heavy chain variable region (VH) and a light chain variable region (VL) having the VH and VL sequences of BAFFR-6 set forth in Table 19.
720. The combination of embodiment 702 or 703, wherein the BAFFR binding molecule comprises a heavy chain variable region (VH) and a light chain variable region (VL) having the VH and VL sequences of BAFFR-7 set forth in Table 19.
721. The combination of any one of embodiments 1 to 701, wherein the B cell targeting agent is a CD20 binding molecule.
722. The combination of embodiment 721, wherein the CD20 binding molecule is an antibody or antigen-binding domain thereof.
723. The combination of embodiment 721 or embodiment 722, wherein the CD20 binding molecule is rituximab, ofatumumab, ocrelizumab, veltuzumab, or obinutuzumab.
724. The combination of embodiment 723, wherein the CD20 binding molecule is rituximab.
725. The combination of embodiment 723, wherein the CD20 binding molecule is ofatumumab.
726. The combination of embodiment 723, wherein the CD20 binding molecule is ocrelizumab.
727. The combination of embodiment 723, wherein the CD20 binding molecule is veltuzumab.
728. The combination of embodiment 723, wherein the CD20 binding molecule is obinutuzumab.
729. The combination of any one of embodiments 1 to 701, wherein the B cell targeting agent is a CD22 binding molecule.
730. The combination of embodiment 729, wherein the CD22 binding molecule is an antibody or antigen-binding domain thereof.
731. The combination of embodiment 729, wherein the CD22 binding molecule is epratuzumab.
732. The combination of embodiment 729, wherein the CD22 binding molecule is inotuzumab.
733. The combination of embodiment 729, wherein the CD22 binding molecule is inotuzumab ozogamicin.
734. The combination of any one of embodiments 1 to 701, wherein the B cell targeting agent is a BAFF binding molecule.
735. The combination of embodiment 734, wherein the BAFF binding molecule is an antibody or antigen-binding domain thereof.
736. The combination of embodiment 734, wherein the BAFF binding molecule is belimumab, tibulizumab, BR3-Fc, blisibimod or atacicept.
737. The combination of embodiment 736, wherein the BAFF binding molecule is belimumab.
738. The combination of embodiment 736, wherein the BAFF binding molecule is tibulizumab.
739. The combination of embodiment 736, wherein the BAFF binding molecule is BR3-Fc.
740. The combination of embodiment 736, wherein the BAFF binding molecule is blisibimod.
741. The combination of embodiment 736, wherein the BAFF binding molecule is atacicept.
742. The combination of any one of embodiments 1 to 741, further comprising one or more additional agents.
743. The combination of embodiment 742, wherein the one or more additional agents comprises a corticosteroid.
744. The combination of embodiment 743, wherein the corticosteroid is dexamethasone.
745. The combination of embodiment 743, wherein the corticosteroid is prednisone.
746. The combination of any one of embodiments 742 to 745, wherein the one or more additional agents comprise an immunomodulatory imide drug (IMiD).
747. The combination of embodiment 746, wherein the immunomodulatory imide drug (IMiD) is lenalidomide, thalidomide, pomalidomide, or iberdomide.
748. The combination of embodiment 747, wherein the immunomodulatory imide drug (IMiD) is lenalidomide.
749. The combination of any one of embodiments 1 to 748, wherein the amount of the B cell targeting agent in the combination is effective, when administered to a subject, to reduce the likelihood of the subject developing CRS following administration of the anti-CD19 agent.
750. The combination of any one of embodiments 1 to 748, wherein the amount of the B cell targeting agent in the combination is effective, when administered to a subject, to reduce the severity of one or more symptoms of CRS in the subject following administration of the anti-CD19 agent compared to the severity of the one or more symptoms in the absence of the B cell targeting agent.
751. The combination of any one of embodiments 1 to 750, wherein the anti-CD19 agent and B cell targeting agent are separate molecules.
752. The combination of any one of embodiments 1 to 751, wherein the anti-CD19 agent and B cell targeting agent are formulated in separate pharmaceutical compositions.
753. The combination of embodiment 752, wherein the anti-CD19 agent and/or B cell targeting agent are formulated in unit dosage forms.
754. The combination of any one of embodiments 1 to 753, for use in a method of treating a subject having a B cell malignancy.
755. A method of treating a subject having a B cell malignancy, comprising administering the combination of any one of embodiments 1 to 753 to the subject.
756. The combination for use according to embodiment 754 or the method of embodiment 755, wherein the method comprises administering the B cell targeting agent to the subject one or more times prior to administering the anti-CD19 agent to the subject for the first time.
757. The combination for use or method of any one of embodiments 754 to 756, wherein the method comprises administering the B cell targeting agent to the subject a single time prior to administering the anti-CD19 agent to the subject for the first time.
758. The combination for use or method of any one of embodiments 754 to 756, wherein the method comprises administering the B cell targeting agent to the subject more than one time prior to administering the anti-CD19 agent to the subject for the first time.
759. The combination for use or method of any one of embodiments 754 to 758, wherein the B cell malignancy is a B cell malignancy that expresses cell surface CD19.
760. The combination for use or method of any one of embodiments 754 to 759, wherein the B cell malignancy is a hematological cancer.
761. The combination for use or method of any one of embodiments 754 to 759, wherein the B cell malignancy is a malignant lymphoproliferative condition
762. The combination for use or method of any one of embodiments 754 to 759, wherein the B cell malignancy is a plasma cell dyscrasia.
763. The combination for use or method of any one of embodiments 754 to 759, wherein the B cell malignancy is an acute leukemia.
764. The combination for use or method of any one of embodiments 754 to 759, wherein the B cell malignancy is B cell acute lymphocytic leukemia (B-ALL).
765. The combination for use or method of any one of embodiments 754 to 759, wherein the B cell malignancy is relapsed and/or refractory B cell acute lymphocytic leukemia (B-ALL).
766. The combination for use or method of any one of embodiments 754 to 759, wherein the B cell malignancy is a non-Hodgkin's lymphoma (NHL).
767. The combination for use or method of any one of embodiments 754 to 759, wherein the B cell malignancy is a relapsed and/or refractory non-Hodgkin's lymphoma (NHL).
768. The combination for use or method of any one of embodiments 754 to 759, wherein the B cell malignancy is chronic lymphocytic leukemia (CLL)/small lymphocytic lymphoma (SLL).
769. The combination for use or method of any one of embodiments 754 to 759, wherein the B cell malignancy is relapsed and/or refractory chronic lymphocytic leukemia (CLL)/small lymphocytic lymphoma (SLL).
770. The combination for use or method of any one of embodiments 754 to 759, wherein the B cell malignancy is follicular lymphoma (FL), optionally wherein the FL is small cell FL or large cell FL.
771. The combination for use or method of any one of embodiments 754 to 759, wherein the B cell malignancy is relapsed and/or refractory follicular lymphoma (FL), optionally wherein the FL is small cell FL or large cell FL.
772. The combination for use or method of any one of embodiments 754 to 759, wherein the B cell malignancy is mantle cell lymphoma (MCL).
773. The combination for use or method of any one of embodiments 754 to 759, wherein the B cell malignancy is relapsed and/or refractory mantle cell lymphoma (MCL).
774. The combination for use or method of any one of embodiments 754 to 759, wherein the B cell malignancy is diffuse large B-cell lymphoma (DLBCL).
775. The combination for use or method of any one of embodiments 754 to 759, wherein the B cell malignancy is relapsed and/or refractory diffuse large B-cell lymphoma (DLBCL).
776. The combination for use or method of any one of embodiments 754 to 759, wherein the B cell malignancy is Burkitt lymphoma.
777. The combination for use or method of any one of embodiments 754 to 759, wherein the B cell malignancy is lymphoplasmacytic lymphoma (Waldenstrom macroglobulinemia).
778. The combination for use or method of any one of embodiments 754 to 759, wherein the B cell malignancy is MALT lymphoma (mucosa-associated lymphoid tissue lymphoma).
779. The combination for use or method of any one of embodiments 754 to 759, wherein the B cell malignancy is marginal zone lymphoma (MZL).
780. The combination for use or method of any one of embodiments 754 to 759, wherein the B cell malignancy is extranodal marginal zone lymphoma (EMZL).
781. The combination for use or method of any one of embodiments 754 to 759, wherein the B cell malignancy is nodal marginal zone B-cell lymphoma (NZML).
782. The combination for use or method of any one of embodiments 754 to 759, wherein the B cell malignancy is splenic marginal zone B-cell lymphoma (SMZL).
783. The combination for use or method of any one of embodiments 766 to 782, wherein the subject has failed at least one prior line of standard of care therapy.
784. The combination for use or method of embodiment 783, wherein the subject has failed up to five prior lines of standard of care therapies.
785. The combination for use or method of embodiment 783 or embodiment 784, wherein the subject has failed one prior line of standard of care therapy.
786. The combination for use or method of embodiment 783 or embodiment 784, wherein the subject has failed two prior lines of standard of care therapy.
787. The combination for use or method of embodiment 783 or embodiment 784, wherein the subject has failed three prior lines of standard of care therapy.
788. The combination for use or method of embodiment 783 or embodiment 784, wherein the subject has failed four prior lines of standard of care therapy.
789. The combination for use or method of embodiment 783 or embodiment 784, wherein the subject has failed five prior lines of standard of care therapy.
790. The combination for use or method of any one of embodiments 783 to 789, wherein the at least one prior line of standard of care therapies comprise an anti-CD20 therapy.
791. The combination for use or method of embodiment 790, wherein the anti-CD20 therapy is rituximab.
792. The combination for use or method of any one of embodiments 783 to 791, wherein the subject is intolerant to or ineligible for one or more other approved therapies.
793. The combination for use or method of embodiment 792, wherein the one or more other approved therapies comprise an autologous stem cell transplant (ASCT).
794. The combination for use or method of any one of embodiments 783 to 793, wherein the subject is a non-responder to a CAR composition.
795. The combination for use or method of embodiment 794, wherein the CAR composition is an anti-CD19 CAR composition.
796. The combination for use or method of embodiment 794 or embodiment 795, wherein the CAR composition comprises CTL019, tisagenlecleucel, axicabtagene ciloleucel, brexucabtagene autoleucel or lisocabtagene maraleucel.
797. The combination for use or method of any one of embodiments 794 to 796, wherein the anti-CD19 agent does not comprise a chimeric antigen receptor and/or is not a CAR composition.
798. The combination for use or method of any one of embodiments 754 to 759, wherein the B cell malignancy is a Hodgkin's lymphoma.
799. The combination for use or method of any one of embodiments 754 to 759, wherein the B cell malignancy is multiple myeloma.
800. The combination for use or method of any one of embodiments 754 to 759, wherein the B cell malignancy is hairy cell leukemia.
801. The combination for use or method of any one of embodiments 754 to 759, wherein the B cell malignancy is primary effusion lymphoma.
802. The combination for use or method of any one of embodiments 754 to 759, wherein the B cell malignancy is B cell prolymphocytic leukemia.
803. The combination for use or method of any one of embodiments 754 to 759, wherein the B cell malignancy is is plasmablastic lymphoma.
804. The combination for use or method of any one of embodiments 754 to 759, wherein the B cell malignancy is follicle center lymphoma.
805. The combination for use or method of any one of embodiments 754 to 759, wherein the B cell malignancy is precursor B-lymphoblastic leukemia.
806. The combination for use or method of any one of embodiments 754 to 759, wherein the B cell malignancy is high-grade B-cell lymphoma.
807. The combination for use or method of any one of embodiments 754 to 759, wherein the B cell malignancy is primary mediastinal large B-cell lymphoma.
808. An anti-CD19 agent for use as a medicine in combination with a B cell targeting agent.
809. An anti-CD19 agent for use in the treatment of a B cell malignancy in combination with a B cell targeting agent.
810. The anti-CD19 agent for use according to embodiment 808 or embodiment 809, wherein the anti-CD19 agent is a CD19 agent described in any one of embodiments 2 to 700.
811. The anti-CD19 agent for use according to any one of embodiments 808 to 810, wherein the B cell targeting agent is a B cell targeting agent described in any one of embodiments 701 to 741.
812. The anti-CD19 agent for use according to any one of embodiments 809 to 811, wherein the B cell malignancy is a B cell malignancy described in any one of embodiments 759 to 807.
813. A B cell targeting agent for use as a medicine in combination with an anti-CD19 agent.
814. A B cell targeting agent for use in the treatment of a B cell malignancy in combination with an anti-CD19 agent.
815. The B cell targeting agent for use according to embodiment 813 or embodiment 814, wherein the anti-CD19 agent is a CD19 agent described in any one of embodiments 2 to 700.
816. The B cell targeting agent for use according to any one of embodiments 813 to 815, wherein the B cell targeting agent is a B cell targeting agent described in any one of embodiments 701 to 741.
817. The B cell targeting agent for use according to any one of embodiments 814 to 816, wherein the B cell malignancy is a B cell malignancy described in any one of embodiments 759 to 807.
818. Use of an anti-CD19 agent in the manufacture of a medicament for treating a B cell malignancy, wherein the medicament is for administration in combination with a B cell targeting agent.
819. The use of embodiment 818, wherein the anti-CD19 agent is a CD19 agent described in any one of embodiments 2 to 700.
820. The use according to any one of embodiments 818 to 819, wherein the B cell targeting agent is a B cell targeting agent described in any one of embodiments 701 to 741.
821. The use according to any one of embodiments 809 to 811, wherein the B cell malignancy is a B cell malignancy described in any one of embodiments 759 to 807.
822. Use of a B cell targeting agent in the manufacture of a medicament for treating a B cell malignancy, wherein the medicament is for administration in combination with an anti-CD19 agent.
823. The use according to embodiment 822, wherein the anti-CD19 agent is a CD19 agent described in any one of embodiments 2 to 700.
824. The use according to any one of embodiments 822 to 823, wherein the B cell targeting agent is a B cell targeting agent described in any one of embodiments 701 to 741.
825. The use according to any one of embodiments 822 to 824, wherein the B cell malignancy is a B cell malignancy described in any one of embodiments 759 to 807.
826. A chimeric antigen receptor (“CAR”) molecule that binds CD19, comprising an anti-CD19 binding domain, a transmembrane domain, and an intracellular signaling domain, wherein the anti-CD19 binding domain comprises CDR-H1, CDR-H2, and CDR-H3 having the amino acid sequences of SEQ ID NO:1, SEQ ID NO:2, and SEQ ID NO:3, and CDR-L1, CDR-L2, and CDR-L3 having the amino acid sequences of SEQ ID NO:14, SEQ ID NO:15, and SEQ ID NO:16.
827. A chimeric antigen receptor (“CAR”) molecule that binds CD19, comprising an anti-CD19 binding domain, a transmembrane domain, and an intracellular signaling domain, wherein the anti-CD19 binding domain comprises CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19.
828. A chimeric antigen receptor (“CAR”) molecule that binds CD19, comprising an anti-CD19 binding domain, a transmembrane domain, and an intracellular signaling domain, wherein the anti-CD19 binding domain comprises CDR-H1, CDR-H2, and CDR-H3 having the amino acid sequences of SEQ ID NO:7, SEQ ID NO:8, and SEQ ID NO:9, and CDR-L1, CDR-L2, and CDR-L3 having the amino acid sequences of SEQ ID NO:20, SEQ ID NO:21, and SEQ ID NO:22.
829. A chimeric antigen receptor (“CAR”) molecule that binds CD19, comprising an anti-CD19 binding domain, a transmembrane domain, and an intracellular signaling domain, wherein the anti-CD19 binding domain comprises CDR-H1, CDR-H2, and CDR-H3 having the amino acid sequences of SEQ ID NO:10, SEQ ID NO:11, and SEQ ID NO:12, and CDR-11, CDR-L2, and CDR-L3 having the amino acid sequences of SEQ ID NO:23, SEQ ID NO:24, and SEQ ID NO:25.
830. The CAR molecule of any one of embodiments 826 to 829, wherein the anti-CD19 binding domain comprises a VH having the amino acid sequence of SEQ ID NO:13 and/or a VL having the amino acid sequence of SEQ ID NO:26.
831. A chimeric antigen receptor (“CAR”) molecule that binds CD19, comprising an anti-CD19 binding domain, a transmembrane domain, and an intracellular signaling domain, wherein the anti-CD19 binding domain comprises CDR-H1, CDR-H2, and CDR-H3 having the amino acid sequences of SEQ ID NO:27, SEQ ID NO:28, and SEQ ID NO:29, and CDR-11, CDR-L2, and CDR-L3 having the amino acid sequences of SEQ ID NO:40, SEQ ID NO:41, and SEQ ID NO:42.
832. A chimeric antigen receptor (“CAR”) molecule that binds CD19, comprising an anti-CD19 binding domain, a transmembrane domain, and an intracellular signaling domain, wherein the anti-CD19 binding domain comprises CDR-H1, CDR-H2, and CDR-H3 having the amino acid sequences of SEQ ID NO:30, SEQ ID NO:31, and SEQ ID NO:32, and CDR-11, CDR-L2, and CDR-L3 having the amino acid sequences of SEQ ID NO:43, SEQ ID NO:44, and SEQ ID NO:45.
833. A chimeric antigen receptor (“CAR”) molecule that binds CD19, comprising an anti-CD19 binding domain, a transmembrane domain, and an intracellular signaling domain, wherein the anti-CD19 binding domain comprises CDR-H1, CDR-H2, and CDR-H3 having the amino acid sequences of SEQ ID NO:33, SEQ ID NO:34, and SEQ ID NO:35, and CDR-11, CDR-L2, and CDR-L3 having the amino acid sequences of SEQ ID NO:46, SEQ ID NO:47, and SEQ ID NO:48.
834. A chimeric antigen receptor (“CAR”) molecule that binds CD19, comprising an anti-CD19 binding domain, a transmembrane domain, and an intracellular signaling domain, wherein the anti-CD19 binding domain comprises CDR-H1, CDR-H2, and CDR-H3 having the amino acid sequences of SEQ ID NO:36, SEQ ID NO:37, and SEQ ID NO:38, and CDR-11, CDR-L2, and CDR-L3 having the amino acid sequences of SEQ ID NO:49, SEQ ID NO:50, and SEQ ID NO:51.
835. The CAR molecule of any one of embodiments 831 to 834, wherein the anti-CD19 binding domain comprises a VH having the amino acid sequence of SEQ ID NO: 39 and/or a VL having the amino acid sequence of SEQ ID NO: 52.
836. The CAR molecule of any one of embodiments 826 to 835, wherein the intracellular signaling domain comprises a costimulatory domain and a primary signaling domain.
837. The CAR of any one of embodiments 826 to 836, wherein the CAR molecule comprises an scFv.
838. The CAR molecule of embodiment 837, wherein the anti-CD19 binding domain is a scFv comprising a heavy chain variable region (VH) attached to a heavy chain light region (VL) via a linker.
839. The CAR molecule of any one of embodiments 826 to 838, wherein the CAR molecule comprises a transmembrane domain of a protein chosen from: the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137 and CD154.
840. The CAR molecule of any one of embodiments 826 to 839, wherein the CAR molecule comprises a hinge region.
841. The CAR molecule of any one of embodiments 826 to 840, wherein the CAR molecule comprises a costimulatory domain that is a functional signaling domain.
842. The CAR molecule of embodiment 841, wherein the costimulatory domain has an amino acid sequence from OX40, CD2, CD27, CD28, CDS, ICAM-1, LFA-1 (CD11a/CD18), ICOS (CD278), or 4-11BE (CD137).
843. The CAR molecule of any one of embodiments 826 to 842, wherein the CAR molecule comprises an intracellular signaling domain comprising a functional signaling domain of 4-1BE and/or a functional signaling domain of CD3-zeta.
844. The CAR molecule of any one of embodiments 826 to 843, wherein the CAR molecule comprises a leader sequence.

### 10. INCORPORATION BY REFERENCE

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there are any inconsistencies between the teachings of one or more of the references incorporated herein and the present disclosure, the teachings of the present specification are intended.

1.-120. (canceled)

121. A combination comprising:
a) a CD19 binding antibody, or antigen-binding domain thereof, comprising
(i) CDR-H1, CDR-H2, and CDR-H3 having the amino acid sequences of SEQ ID NO:1, SEQ ID NO:2, and SEQ ID NO:3, and CDR-L1, CDR-L2, and CDR-L3 having the amino acid sequences of SEQ ID NO:14, SEQ ID NO:15, and SEQ ID NO:16;
(ii) CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19;
(iii) CDR-H1, CDR-H2, and CDR-H3 having the amino acid sequences of SEQ ID NO:7, SEQ ID NO:8, and SEQ ID NO:9, and CDR-L1, CDR-L2, and CDR-L3 having the amino acid sequences of SEQ ID NO:20, SEQ ID NO:21, and SEQ ID NO:22;
(iv) CDR-H1, CDR-H2, and CDR-H3 having the amino acid sequences of SEQ ID NO:10, SEQ ID NO:11, and SEQ ID NO:12, and CDR-L1, CDR-L2, and CDR-L3 having the amino acid sequences of SEQ ID NO:23, SEQ ID NO:24, and SEQ ID NO:25
(v) CDR-H1, CDR-H2, and CDR-H3 having the amino acid sequences of SEQ ID NO:27, SEQ ID NO:28, and SEQ ID NO:29, and CDR-L1, CDR-L2, and CDR-L3 having the amino acid sequences of SEQ ID NO:40, SEQ ID NO:41, and SEQ ID NO:42;
(vi) CDR-H1, CDR-H2, and CDR-H3 having the amino acid sequences of SEQ ID NO:30, SEQ ID NO:31, and SEQ ID NO:32, and CDR-L1, CDR-L2, and CDR-L3 having the amino acid sequences of SEQ ID NO:43, SEQ ID NO:44, and SEQ ID NO:45;
(vii) CDR-H1, CDR-H2, and CDR-H3 having the amino acid sequences of SEQ ID NO:33, SEQ ID NO:34, and SEQ ID NO:35, and CDR-L1, CDR-L2, and CDR-L3 having the amino acid sequences of SEQ ID NO:46, SEQ ID NO:47, and SEQ ID NO:48; or
(viii) CDR-H1, CDR-H2, and CDR-H3 having the amino acid sequences of SEQ ID NO:36, SEQ ID NO:37, and SEQ ID NO:38, and CDR-L1, CDR-L2, and CDR-L3 having the amino acid sequences of SEQ ID NO:49, SEQ ID NO:50, and SEQ ID NO:51; and,
b) a BAFF receptor (BAFFR) binding antibody, or antigen-binding domain thereof, comprising the CDR sequences of CDR-H1, CDR-H2, CDR-H3 having the amino acid sequences of SEQ ID NO. 53, 54, 55 respectively, and CDR-L1, CDR-L2, and CDR-L3 having the amino acid sequences of SEQ ID NO. 56, 57 and 58 respectively.

122. The combination of claim 121, wherein the CD19 binding molecule comprises CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19.

123. The combination of claim 121, wherein the CD19 binding molecule comprises CDR-H1, CDR-H2, and CDR-H3 having the amino acid sequences of SEQ ID NO:30, SEQ ID NO:31, and SEQ ID NO:32, and CDR-L1, CDR-L2, and CDR-L3 having the amino acid sequences of SEQ ID NO:43, SEQ ID NO:44, and SEQ ID NO:45.

124. The combination of claim 121, wherein the CD19 binding molecule comprises a VH having the amino acid sequence of SEQ ID NO:13 and/or a VL having the amino acid sequence of SEQ ID NO:26.

125. The combination of claim 121, wherein the CD19 binding molecule comprises a VH having the amino acid sequence of SEQ ID NO:39 and/or a VL having the amino acid sequence of SEQ ID NO:52.

126. The combination of claim 121, wherein the CD19 binding molecule is a multispecific binding molecule (MBM) comprising an antigen-binding module 1 (ABM1) that binds specifically to CD19, and an antigen-binding module 2 (ABM2) that binds specifically to CD3

127. The combination of claim 126, wherein ABM2 comprises the CDR sequences of VH CDR1, VH CDR2 and VH CDR3 of SEQ ID NO. 1287, 1290, 1292 respectively and VL CDR1, VL CDR2, VL CDR3 of SEQ ID NO. 1288, 1291 and 1293 respectively.

128. The combination of claim 127, wherein ABM2 comprises the heavy and light chain variable sequences of SEQ ID No 1279 and 1280 respectively

129. The combination of claim 126, wherein ABM2 comprises the scFv of SEQ ID NO. 1281.

130. The combination of claim 126, where the CD19 binding molecule is a bispecific binding molecule (BBM).

131. The combination of claim 126, wherein the CD19 binding molecule is a trispecific binding molecule (TBM) comprising an antigen-binding module 3 (ABM3) that binds specifically to (i) human CD2 or (ii) a tumor associated antigen (TAA).

132. The combination of claim 131, wherein ABM3 binds specifically to human CD2.

133. The combination of claim 132, wherein ABM3 is a CD58 moiety.

134. The combination of claim 133, wherein the CD58 moiety comprises the amino acid sequence of SEQ ID NO. 1315.

135. The combination of claim 126, wherein the CD19 binding molecule comprises a first variant Fc region and a second variant Fc region that together form an Fc heterodimer.

136. A combination comprising:
a) a BAFF receptor (BAFFR) binding molecule, which is an antibody or antigen-binding domain thereof and comprises the CDR sequences of CDR-H1, CDR-H2, CDR-H3 having the amino acid sequences of SEQ ID NO. 53, 54, 55 respectively, and CDR-L1, CDR-L2, and CDR-L3 having the amino acid sequences of SEQ ID NO. 56, 57 and 58 respectively, and
b) a CD19 binding molecule comprising:
(i) an antigen-binding module 1 (ABM1) that binds specifically to CD19 and comprises CDR-H1, CDR-H2, and CDR-H3 having the amino acid sequences of SEQ ID NO:4, SEQ ID NO:5, and SEQ ID NO:6, and CDR-L1, CDR-L2, and CDR-L3 having the amino acid sequences of SEQ ID NO:17, SEQ ID NO:18, and SEQ ID NO:19 and wherein ABM1 is a Fab;
(ii) an antigen-binding module 2 (ABM2) that binds specifically to CD3 and wherein ABM2 comprises the amino acid sequence of the scFv of SEQ ID NO. 1281
(iii) an antigen-binding module 3 (ABM3) that binds specifically to human CD2 and which comprises the amino acid sequence of SEQ ID NO. 1315 and
(iv) an Fc domain.

137. The combination of claim 136, wherein ABM1 comprises a VH having the amino acid sequence of SEQ ID NO:13 and a VL having the amino acid sequence of SEQ ID NO:26.

138. The combination of claim 136, wherein Fc binding domain of the CD19 binding molecule comprises a first variant Fc region and a second variant Fc region that together form an Fc heterodimer.

139. The combination of claim 138, wherein the first variant Fc region is a variant human IgG1 Fc region and the second variant Fc region is a variant human IgG1 Fc region, wherein the first and second variant Fc regions comprise L234A, L235A, and G237A (“LALAGA”) substitutions, L234A, L235A, S267K, and P329A (“LALASKPA”) substitutions, D265A, P329A, and S267K (“DAPASK”) substitutions, G237A, D265A, and P329A (“GADAPA”) substitutions, G237A, D265A, P329A, and S267K (“GADAPASK”) substitutions, L234A, L235A, and P329G (“LALAPG”) substitutions, or L234A, L235A, and P329A (“LALAPA”) substitutions, wherein the amino acid residues are numbered according to the EU numbering system.

140. The combination of claim 136, wherein the Fc domain of the CD19 binding molecule is a human IgG1 Fc domain which comprises:
(a) a first CH3 domain comprising the modification T366W; and
(b) a second CH3 domain that heterodimerizes with the first CH3 domain and comprises the modifications T366S, L368A and Y407V.

141. The combination of claim 136, wherein the Fc domain of the CD19 binding molecule is a human IgG1 Fc domain which comprises:
(a) a first CH3 domain comprising the modification S354C; and
(b) a second CH3 domain comprises the modification Y349C.

142. The combination of claim 136, wherein the CD19 binding molecule comprises a first half antibody comprising a) the antigen-binding module 1 (ABM1) that binds specifically to CD19; b) the antigen-binding module 2 (ABM2) that binds specifically to CD3 and d) the Fc region; and a second half antibody comprising c) the antigen-binding module 3 (ABM3) and d) an Fc region, wherein the Fc region in the first half antibody and the Fc region in the second half antibody form a Fc heterodimer.

143. A combination comprising:
a) a CD19 binding molecule comprising
(i) a first half antibody heavy chain whose amino acid sequence comprises the amino acid sequence of SEQ ID NO:63 and a Fc sequence;
(ii) a first half antibody light chain whose amino acid sequence comprises the amino acid sequence of SEQ ID NO:64;
(iii) a second half antibody whose amino acid sequence comprises the amino acid sequence of SEQ ID NO:65 and a Fc sequence; and,
b) a BAFF receptor (BAFFR) binding molecule, which is an antibody or antigen-binding domain thereof and comprises the CDR sequences of CDR-H1, CDR-H2, CDR-H3 having the amino acid sequences of SEQ ID NO. 53, 54, 55 respectively, and CDR-L1, CDR-L2, and CDR-L3 having the amino acid sequences of SEQ ID NO. 56, 57 and 58 respectively.

144. A combination comprising:
a) a CD19 binding molecule comprising
(i) a first polypeptide whose amino acid sequence comprises the amino acid sequence of SEQ ID NO:74;
(ii) a second polypeptide whose amino acid sequence comprises the amino acid sequence of SEQ ID NO:64;
(iii) a third polypeptide whose amino acid sequence comprises the amino acid sequence of SEQ ID NO:75 or SEQ ID NO:86; and,
b) a BAFF receptor (BAFFR) binding molecule, which is an antibody or antigen-binding domain thereof and comprises the CDR sequences of CDR-H1, CDR-H2, CDR-H3 having the amino acid sequences of SEQ ID NO. 53, 54, 55 respectively, and CDR-L1, CDR-L2, and CDR-L3 having the amino acid sequences of SEQ ID NO. 56, 57 and 58 respectively,

145. A combination comprising:
a) a CD19 binding molecule comprising
(i) a first polypeptide whose amino acid sequence comprises the amino acid sequence of SEQ ID NO:1127;
(ii) a second polypeptide whose amino acid sequence comprises the amino acid sequence of SEQ ID NO:1129;
(iii) a third polypeptide whose amino acid sequence comprises the amino acid sequence of SEQ ID NO:1110; and,
b) a BAFF receptor (BAFFR) binding molecule, which is an antibody or antigen-binding domain thereof and comprises the CDR sequences of CDR-H1, CDR-H2, CDR-H3 having the amino acid sequences of SEQ ID NO. 53, 54, 55 respectively, and CDR-L1, CDR-L2, and CDR-L3 having the amino acid sequences of SEQ ID NO. 56, 57 and 58 respectively.

146. The combination of claim 121, wherein the BAFFR binding antibody, or antigen-binding domain thereof, comprises a heavy chain variable region (VH) and a light chain variable region (VL) having the VH and VL sequences of. SEQ ID NO. 59 and 60 respectively.

147. The combination of claim 146, wherein the BAFFR binding antibody, or antigen-binding domain thereof, comprises a heavy chain sequence of SEQ ID NO: 61 and a light chain sequence of SEQ ID NO: 62 respectively.

148. The combination of claim 147, wherein the BAFFR binding antibody is ianalumab.

149. The combination of claim 121, comprising one or more additional agents.

150. The combination of claim 149, wherein the one or more additional agents comprise a corticosteroid.

151. A method of treating a subject having a B cell malignancy, comprising administering the combination of claim 1 to the subject.

152. The method of claim 151, wherein the method comprises administering the BAFF receptor (BAFFR) binding molecule to the subject one or more times prior to administering the CD19 binding molecule to the subject for the first time.

153. The method of claim 151, wherein the method comprises administering the BAFF receptor (BAFFR) binding molecule to the subject a single time prior to administering the CD19 binding molecule to the subject for the first time.

154. The method of claim 151, wherein the method comprises administering the BAFF receptor (BAFFR) binding molecule to the subject more than one time prior to administering the CD19 binding molecule to the subject for the first time.

155. The method of claim 151, wherein the method comprises administering simultaneously the BAFF receptor (BAFFR) binding molecule and the CD19 binding molecule to the subject.

156. The method of claim 151, wherein the disease or disorder is selected from the group consisting of diffuse large B-cell lymphoma (DLBCL), relapsed and/or refractory DLBCL, acute lymphoblastic leukemia (ALL), mantle cell lymphoma (MCL), and Burkitt's lymphoma.

157. The method of claim 151, wherein the method comprises administering the BAFF receptor (BAFFR) binding molecule prior to administering the CD19 binding molecule to the subject.
